SEQUENCE LISTING (193 sequences)

SEQ ID NO: 1 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 2 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 3 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 8: note = N6-(4(carboxymethyl)piperazine-1-carbonyl-N2-methyl -L-lysine
  SITE 10: note = D-Proline
GGXYXWGXDP

SEQ ID NO: 4 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 8: note = Orn
  MOD_RES 8: note = (S)-2-amino-5-(4-(carboxymethyl)piperazine-1-carbox amido)pentanoic acid
  SITE 10: note = D-Proline
GGXYXWGXDP

SEQ ID NO: 5 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 8: note = (S)-2-amino-4-(4-(carboxymethyl)piperazine-1-carbox amido)butanoic acid
  SITE 10: note = D-Proline
GGXYXWGXDP

SEQ ID NO: 6 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(2-aminoethoxy)phenyl)propanoic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 7 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 8 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)ph enyl)propanoic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
EGXXXWGXDP

SEQ ID NO: 9 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
EGXXXXGXDP

SEQ ID NO: 10 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
XGXXXWGXDP

SEQ ID NO: 11 - protein, 10 aa, synthetic construct
  REGION 1..10: note = Amino acid sequence
  REGION 1..10: note = Peptide is circular
  MOD_RES 1: note = L-homoserine
  MOD_RES 3: note = (S)-2-(methylamino)-3-(pyridine-3-yl)propanoic acid
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
XGXXXXGXDP

SEQ ID NO: 12 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(aminomethyl)phenyl)propanoic acid
  MOD_RES 5: note = (S)-2-aminoheptanoic acid
  SITE 10: note = D-Proline
GGXXXWGYDP

SEQ ID NO: 13 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 8: note = (S)-2-amino-3-(4-methoxyphenyl)propanoic acid
  SITE 10: note = D-Proline
GGXYXWGXDP

SEQ ID NO: 14 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 8: note = (S)-2-amino-3-hydroxyphenyl)propanoic acid
  SITE 10: note = D-Proline
GGXYXWGXDP

SEQ ID NO: 15 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
PGXXXWGXDP

SEQ ID NO: 16 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 17 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((4-(aminomethyl)-1H-1,2,3-triazol -1-yl)methyl)phenyl)propanoic acid
  MOD_RES 5: note = (S)-2-aminoheptanoic acid
  SITE 10: note = D-Proline
GGXXXWGYDP

SEQ ID NO: 18 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
DGXYXWGYDP

SEQ ID NO: 19 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
SGXYXWGYDP

SEQ ID NO: 20 - protein, 10 aa, synthetic construct
  REGION 1..10: note = Amino acid sequence
  REGION 1..10: note = Peptide is circular
  MOD_RES 5: note = (S)-2-aminoheptanoic acid
  SITE 10: note = D-Proline
KEFYXWGYDP

SEQ ID NO: 21 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
KEXYXWGYDP

SEQ ID NO: 22 - protein, 10 aa, synthetic construct
  REGION 1..10: note = Amino acid sequence
  SITE 1..10: note = Peptide is circular
  MOD_RES 5: note = (S)-2-aminoheptanoic acid
  SITE 10: note = D-Proline
KGFYXWGYDP

SEQ ID NO: 23 - protein, 10 aa, synthetic construct
  REGION 1..10: note = Amino acid sequence
  SITE 1..10: note = Peptide is circular
  MOD_RES 3: note = methyl-L-phenylalanine
  MOD_RES 5: note = (S)-2-aminoheptanoic acid
  MOD_RES 6: note = (S)-2-amino-3-(7-fluoro-1H-indol-3-yl)propanoic acid
  SITE 10: note = D-Proline
KGXYXXGYDP

SEQ ID NO: 24 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 6: note = (S)-2-amino-3-(6-hydroxy-1H-indol-3-yl)propanoic acid
  SITE 10: note = D-Proline
KGXYXXGYDP

SEQ ID NO: 25 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 6: note = (S)-2-amino-3-(7-chloro-1H-indol-3-yl)propanoic acid
  SITE 10: note = D-Proline
KGXYXXGYDP

SEQ ID NO: 26 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 6: note = (S)-2-amino-3-(1H-pyrrolo[2,3-b]pyridin-3-yl)propan oic acid
  SITE 10: note = D-Proline
KGXYXXGYDP

SEQ ID NO: 27 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminoheptanoic acid
  SITE 10: note = D-Proline
GGXXXWGYDP

SEQ ID NO: 28 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-3-(4-(acetamidomethyl)phenyl)-2-aminopropanoic acid
  MOD_RES 5: note = (S)-2-aminoheptanoic acid
  SITE 10: note = D-Proline
GGXXXWGYDP

SEQ ID NO: 29 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-amino-5-((S)-2-cyano-4,4-difluoropyrrolidiny- 1-yl-5-oxopentanoic acid
  SITE 10: note = D-Proline
KGXYXWGYDP

SEQ ID NO: 30 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-amino-5-(3,3-difluoropyrrolidin-1-yl)-5-oxope ntanoic acid
  SITE 10: note = D-Proline
KGXYXWGYDP

SEQ ID NO: 31 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminooctanoic acid
  SITE 10: note = D-Proline
KGXYXWGYDP

SEQ ID NO: 32 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(2-aminoethoxy)phenyl)propanoic acid
  MOD_RES 5: note = (S)-2-aminoheptanoic acid
  SITE 10: note = D-Proline
PGXXXWGYDP

SEQ ID NO: 33 - protein, 10 aa, synthetic construct
  REGION 1..10: note = Amino acid sequence
  REGION 1..10: note = Peptide is circular
  MOD_RES 1: note = (2S,4R)-4-hydroxypyrrolidine-2-carboxylic acid
  MOD_RES 3: note = methyl-L-phenylalanine
  MOD_RES 4: note = (S)-2-amino-3-(4-(2-aminoethoxy)phenyl)propanoic acid
  MOD_RES 5: note = (S)-2-aminoheptanoic acid
  SITE 10: note = D-Proline
XGXXXWGYDP

SEQ ID NO: 34 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (2S,3R)-2-amino-3-hydroxyoctanoic acid
  SITE 10: note = D-Proline
GGXYXWGYDP

SEQ ID NO: 35 - protein, 10 aa, synthetic construct
  REGION 1..10: note = Amino acid sequence
  REGION 1..10: note = Peptide is circular
  SITE 2: note = D-Alanine
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminooctanoic acid
  MOD_RES 8: note = (S)-2-amino-3-(4-methoxyphenyl)propanoic acid
  SITE 10: note = D-Proline
GGXXXWGXDP

SEQ ID NO: 36 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminooctanoic acid
  MOD_RES 8: note = (S)-2-amino-3-hydroxyphenyl)propanoic acid
  SITE 10: note = D-Proline
GGXXXWGXDP

SEQ ID NO: 37 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-amino-5-((S)-2-cyano-4,4-difluoropyrrolidiny- 1-yl-5-oxopentanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 38 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(aminomethyl)phenyl)propanoic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
PGXXXWGXDP

SEQ ID NO: 39 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
PGXXXWGXDP

SEQ ID NO: 40 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 41 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 10: note = (2R,4S)-4-hydroxypyrrolidine-2-carboxylic acid
  SITE 10: note = D-(2R,4S)-4-hydroxypyrrolidine-2-carboxylic acid
KGXYXWGYDX

SEQ ID NO: 42 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 5: note = (S)-2-aminoheptanoic acid
  SITE 10: note = D-Proline
XGXYXWGYDP

SEQ ID NO: 43 - protein, 10 aa, synthetic construct
  REGION 1..10: note = Amino acid sequence
  REGION 1..10: note = Peptide is circular
  MOD_RES 3: note = (S)-3-cyclohexyl-2-(methylamino)propanoic acid
  MOD_RES 5: note = (S)-2-aminoheptanoic acid
  SITE 10: note = D-Proline
KGXYXWGYDP

SEQ ID NO: 44 - protein, 10 aa, synthetic construct
  REGION 1..10: note = Amino acid sequence
  REGION 1..10: note = Peptide is circular
  MOD_RES 3: note = (S)-3-cyclopentyl-2-(methylamino)propanoic acid
  MOD_RES 5: note = (S)-2-aminoheptanoic acid
  SITE 10: note = D-Proline
KGXYXWGYDP

SEQ ID NO: 45 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(2-aminoethoxy)phenyl)propanoic acid
  MOD_RES 5: note = (S)-2-aminoheptanoic acid
  MOD_RES 8: note = (S)-2-amino-3-(6-aminopyridin-3-yl)propanoic acid
  SITE 10: note = D-Proline
GGXXXWGXDP

SEQ ID NO: 46 - protein, 10 aa, synthetic construct
  REGION 1..10: note = Amino acid sequence
  REGION 1..10: note = Peptide is circular
  MOD_RES 1: note = N6-(4-(carboxymethyl)piperazine-1-carbonyl)-L-lysin e
  MOD_RES 4: note = (S)-2-amino-3-(4-(2-aminoethoxy)phenyl)propanoic acid
  MOD_RES 3: note = methyl-L-phenylalanine
  MOD_RES 5: note = (S)-2-aminoheptanoic acid
  SITE 10: note = D-Proline
XGXXXWGYDP

SEQ ID NO: 47 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 4: note = (S)-2-amino-3-(4-(2-aminoethoxy)phenyl)propanoic acid
  MOD_RES 5: note = (S)-2-aminoheptanoic acid
  SITE 10: note = D-Proline
XGXXXWGYDP

SEQ ID NO: 48 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminooctanoic acid
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 49 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = (S)-2-amino-3-(4-((2-(2-(2-aminoethoxy)ethoxy)aceta mido)methyl)phenyl)propanoic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 50 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 8: note = methyl-L-tyrosine
  SITE 10: note = D-Proline
GGXYXWGXDP

SEQ ID NO: 51 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
PGXYXWGYDP

SEQ ID NO: 52 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
GGXYXWGYDP

SEQ ID NO: 53 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
KGXYXWGYDP

SEQ ID NO: 54 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
GGXYXWGYKP

SEQ ID NO: 55 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 9: note = N6-acetyl-L-lysine
  SITE 10: note = D-Proline
GGXYXWGYXP

SEQ ID NO: 56 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
GGXYXWGYAP

SEQ ID NO: 57 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 9: note = L-homoserine
  SITE 10: note = D-Proline
GGXYXWGYXP

SEQ ID NO: 58 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
GGXYXWGYNP

SEQ ID NO: 59 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(2-aminoethoxy)phenyl)propanoic acid
  MOD_RES 5: note = (S)-2-aminoheptanoic acid
  SITE 10: note = D-Proline
GGXXXWGYDP

SEQ ID NO: 60 - protein, 10 aa, synthetic construct
  REGION 1..10: note = Amino acid sequence
  REGION 1..10: note = Peptide is circular
  MOD_RES 3: note = (S)-2-amino-3-cyclobutylpropanoic acid
  MOD_RES 5: note = (S)-2-aminoheptanoic acid
  SITE 10: note = D-Proline
GGXYXWGYDP

SEQ ID NO: 61 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 6: note = (S)-2-amino-3-(7-methoxy-1H-indol-3-yl)propanoic acid
  SITE 10: note = D-Proline
GGXYXXGYDP

SEQ ID NO: 62 - protein, 10 aa, synthetic construct
  REGION 1..10: note = Amino acid sequence
  REGION 1..10: note = Peptide is circular
  MOD_RES 3: note = (S)-3-cyclobutyl-2-(methylamino)propanoic acid
  MOD_RES 5: note = (S)-2-aminoheptanoic acid
  SITE 10: note = D-Proline
GGXYXWGYDP

SEQ ID NO: 63 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 10: note = (R)-morpholine-3-carboxylic acid
GGXYXWGYDX

SEQ ID NO: 64 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 7: note = D-Alanine
  SITE 10: note = D-Proline
GGXYXWAYDP

SEQ ID NO: 65 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 10: note = methyl-D-alanine
  SITE 10: note = D-methyl-D-alanine
GGXYXWGYDX

SEQ ID NO: 66 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 10: note = (R)-piperidine-2-carboxylic acid
GGXYXWGYDX

SEQ ID NO: 67 - protein, 10 aa, synthetic construct
  REGION 1..10: note = Amino acid sequence
  REGION 1..10: note = Peptide is circular
  SITE 2: note = D-Serine
  MOD_RES 3: note = methyl-L-phenylalanine
  MOD_RES 5: note = (S)-2-aminoheptanoic acid
  SITE 10: note = D-Proline
GSXYXWGYDP

SEQ ID NO: 68 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 10: note = MeGly
GGXYXWGYDX

SEQ ID NO: 69 - protein, 10 aa, synthetic construct
  REGION 1..10: note = Amino acid sequence
  SITE 1: note = D-Serine
  REGION 1..10: note = Peptide is circular
  MOD_RES 3: note = methyl-L-phenylalanine
  MOD_RES 5: note = (S)-2-aminoheptanoic acid
  SITE 10: note = D-Proline
SGXYXWGYDP

SEQ ID NO: 70 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 10: note = (R)-2-methylpyrrolidine-2-carboxylic acid
  SITE 10: note = D-(R)-2-methylpyrrolidine-2-carboxylic acid
GGXYXWGYDX

SEQ ID NO: 71 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  SITE 10: note = D-Proline
GGXYXWGYEP

SEQ ID NO: 72 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 9: note = (S)-2-amino-2-methylsuccinic acid
  SITE 10: note = D-Proline
GGXYXWGYXP

SEQ ID NO: 73 - protein, 10 aa, synthetic construct
  REGION 1..10: note = Amino acid sequence
  REGION 1..10: note = Peptide is circular
  MOD_RES 1: note = MeGly
  MOD_RES 3: note = methyl-L-phenylalanine
  MOD_RES 5: note = (S)-2-aminoheptanoic acid
  SITE 10: note = D-Proline
XGXYXWGYDP

SEQ ID NO: 74 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 8: note = benzylglycine
  SITE 10: note = D-Proline
GGXYXWGXDP

SEQ ID NO: 75 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(aminomethyl)phenyl)propanoic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 76 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-ethynylphenyl)propanoic acid
  MOD_RES 5: note = (S)-2-aminooctanoic acid
  MOD_RES 6: note = 1-(tert-butoxycarbonyl)-L-tryptophan
  MOD_RES 8: note = N6-(4-(2-(tert-butoxy)-2-oxoethyl)piperazine-1-carb onyl)-L-lysine
  MOD_RES 9: note = (S)-2-amino-4-((3-methylpentan-3-yl)oxy)-4-oxobutan oic acid
  SITE 10: note = D-Proline
GGXXXXGXXP

SEQ ID NO: 77 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 78 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo-5,8-dioxa-2,11-diazatridecan-13-yl)-1,4,7,1 0-tetraazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 79 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((2-(4-(2-amino-2-carboxyethyl) phenoxy)ethyl) amino)-2-oxoethyl)-1,4,7,10-tetraazacyclodo decane-1,4,7-triyl)tri acetic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 80 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = (S)-2,2',2''-(10-(1-(4-(2-amino-2-carboxyethyl)phen yl)-3,12-dioxo-5,8-dioxa-2,11-diazatridecan-13-yl)-1,4,7,1 0-tetraazacyclododecane-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 81 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 4: note = (S)-2,2',2''-(10-(1-(4-(2-amino-2-carboxyethyl)phen yl)-3,12-dioxo-5,8-dioxa-2,11-diazatridecan-13-yl)-1,4,7,1 0-tetraazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
XGXXXWGXDP

SEQ ID NO: 82 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo-5,8-dioxa-2,11-diazatridecan-13-yl)-1,4,7,1 0-tetraazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
EGXXXWGXDP

SEQ ID NO: 83 - protein, 10 aa, synthetic construct
  REGION 1..10: note = Amino acid sequence
  REGION 1..10: note = Peptide is circular
  MOD_RES 1: note = L-homoserine
  MOD_RES 3: note = (S)-2-(methylamino)-3-(pyridine-3-yl)propanoic acid
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
XGXXXXGXDP

SEQ ID NO: 84 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
EGXXXXGXDP

SEQ ID NO: 85 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((2-((4-(2-amino-2-carboxyethyl )benzyl)amino) -2-oxoethyl)(methyl)amino)-2-oxoethyl)-1,4, 7,10-tetraazacyclododecane-1,4,7-triyl)triacetic acid
  MOD_RES 5: note = (S)-2-aminoheptanoic acid
  SITE 10: note = D-Proline
GGXXXWGYDP

SEQ ID NO: 86 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((2-((4-(2-amino-2-carboxyethyl )benzyl)amino) -2-oxoethyl)amino)-2-oxoethyl)-1,4,7,10-tet raazacyclododecane-1,4 ,7-triyl)triacetic acid
  MOD_RES 5: note = (S)-2-aminoheptanoic acid
  SITE 10: note = D-Proline
GGXXXWGYDP

SEQ ID NO: 87 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((3-((4-(2-amino-2-carboxyethyl )benzyl)amino) -3-oxopropyl)amino)-2-oxoethyl)-1,4,7,10-te traazacyclododecane-1, 4,7-triyl)triacetic acid
  MOD_RES 5: note = (S)-2-aminoheptanoic acid
  SITE 10: note = D-Proline
GGXXXWGYDP

SEQ ID NO: 88 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((3-((4-(2-amino-2-carboxyethyl )benzyl)amino) -3-oxopropyl)(methyl)amino)-2-oxoethyl)-1,4 ,7,10-tetraazacyclododecane-1,4,7-triyl)triacetic acid
  MOD_RES 5: note = (S)-2-aminoheptanoic acid
  SITE 10: note = D-Proline
GGXXXWGYDP

SEQ ID NO: 89 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
PGXXXWGXDP

SEQ ID NO: 90 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 91 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-(((1-(4-(2-amino-2-carboxyethyl )benzyl)-1H-1, 2,3-triazol-4-yl)methyl)amino)-2-oxoethyl)- 1,4,7,10-tetraazacyclo dodecane-1,4,7-triyl)triacetic acid
  MOD_RES 5: note = (S)-2-aminoheptanoic acid
  SITE 10: note = D-Proline
GGXXXWGYDP

SEQ ID NO: 92 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = (S)-2-aminoheptanoic acid
  SITE 10: note = D-Proline
GGXXXWGYDP

SEQ ID NO: 93 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((4-(2-amino-2-carboxyethyl)ben zyl)amino)-2-o xoethyl)-1,4,7,10-tetraazacyclododecane-1,4 ,7-triyl)triacetic acid
  MOD_RES 5: note = (S)-2-aminoheptanoic acid
  SITE 10: note = D-Proline
GGXXXWGYDP

SEQ ID NO: 94 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = (S)-2-aminooctanoic acid
  MOD_RES 8: note = (S)-2-amino-3-(4-methoxyphenyl)propanoic acid
  SITE 10: note = D-Proline
GGXXXWGXDP

SEQ ID NO: 95 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = (S)-2-aminooctanoic acid
  MOD_RES 8: note = (S)-2-amino-3-hydroxyphenyl)propanoic acid
  SITE 10: note = D-Proline
GGXXXWGXDP

SEQ ID NO: 96 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = (S)-2-amino-5-((S)-2-cyano-4,4-difluoropyrrolidiny- 1-yl-5-oxopentanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 97 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
PGXXXWGXDP

SEQ ID NO: 98 - protein, 10 aa, synthetic construct
  REGION 1..10: note = Amino acid sequence
  REGION 1..10: note = Peptide is circular
  MOD_RES 1: note = (R)-2,2',2''-(10-(2-((5-amino-5-carboxypentyl)amino )-2-oxoethyl)- 1,4,7,10-tetraazacyclododecane-1,4,7-triyl)triacetic acid
  MOD_RES 3: note = methyl-L-phenylalanine
  MOD_RES 5: note = (S)-2-aminoheptanoic acid
  SITE 10: note = D-Proline
XGXYXWGYDP

SEQ ID NO: 99 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((2-(4-(2-amino-2-carboxyethyl) phenoxy)ethyl) amino)-2-oxoethyl)-1,4,7,10-tetraazacyclodo decane-1,4,7-triyl)tri acetic acid
  MOD_RES 5: note = (S)-2-aminoheptanoic acid
  SITE 10: note = D-Proline
GGXXXWGYDP

SEQ ID NO: 100 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(2-((2-(4-(2-amino-2-carboxyethyl) phenoxy)ethyl) amino)-2-oxoethyl)-1,4,7,10-tetraazacyclodo decane-1,4,7-triyl)tri acetic acid
  MOD_RES 5: note = (S)-2-aminoheptanoic acid
  MOD_RES 8: note = (S)-2-amino-3-(6-aminopyridin-3-yl)propanoic acid
  SITE 10: note = D-Proline
GGXXXWGXDP

SEQ ID NO: 101 - protein, 10 aa, synthetic construct
  REGION 1..10: note = Amino acid sequence
  REGION 1..10: note = Peptide is circular
  MOD_RES 1: note = N6-(4-(carboxymethyl)piperazine-1-carbonyl)-L-lysin e
  MOD_RES 3: note = methyl-L-phenylalanine
  MOD_RES 4: note = (S)-2,2',2''-(10-(2-((2-(4-(2-amino-2-carboxyethyl) phenoxy)ethyl) amino)-2-oxoethyl)-1,4,7,10-tetraazacyclodo decane-1,4,7-triyl)tri acetic acid
  MOD_RES 5: note = (S)-2-aminoheptanoic acid
  SITE 10: note = D-Proline
XGXXXWGYDP

SEQ ID NO: 102 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 4: note = (S)-2,2',2''-(10-(2-((2-(4-(2-amino-2-carboxyethyl) phenoxy)ethyl) amino)-2-oxoethyl)-1,4,7,10-tetraazacyclodo decane-1,4,7-triyl)tri acetic acid
  MOD_RES 5: note = (S)-2-aminoheptanoic acid
  SITE 10: note = D-Proline
XGXXXWGYDP

SEQ ID NO: 103 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododeca ne-1,4,7-triyl)triacetic acid
  MOD_RES 5: note = (S)-2-aminooctanoic acid
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 104 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = (S)-2,2',2''-(10-(1-(4-(2-amino-2-carboxyethyl)phen yl)-3,12-dioxo -5,8-dioxa-2,11-diazatridecan-13-yl)-1,4,7, 10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 105 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2'-(7-(4-((2-(4-((S)-2-amino-2-carboxyethyl)pheno xy)ethyl)amino )-1-carboxy-4-oxobutyl)-1,4,7-triazonane-1,4-diyl)diacetic acid
  MOD_RES 5: note = (S)-2-aminoheptanoic acid
  SITE 10: note = D-Proline
GGXXXWGYDP

SEQ ID NO: 106 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2',2''-(10-((R)-4-((2-(4-((S)-2-amino-2-carboxyet hyl)phenoxy)et hyl)amino)-1-carboxy-4-oxobutyl)-1,4,7,10-t etraazacyclododecane-1 ,4,7-triyl)triacetic acid
  MOD_RES 5: note = (S)-2-aminoheptanoic acid
  SITE 10: note = D-Proline
GGXXXWGYDP

SEQ ID NO: 107 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2'-((6-(1-(4-(2-amino-2-carboxyethyl)phenyl)- 3,12-dioxo-5,8 -dioxa-2,11-diazahexadecan-16-yl)-1,4-bis(c arboxymethyl)-1,4-diaz epan-6-yl)azanediyl)diacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 108 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2',2''-(10-((R)-1-(4-((S)-2-amino-2-carboxyethyl) phenyl)-15-car boxy-3,12-dioxo-5,8-dioxa-2,11-diazapentade can-15-yl)-1,4,7,10-te traazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 109 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2'-(7-(1-(4-((S)-2-amino-2-carboxyethyl)phenyl)-1 5-carboxy-3,12 -dioxo-5,8-dioxa-2,11-diazapentadecan-15-yl )-1,4,7-triazonane-1,4 -diyl)diacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 110 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2'-(7-(1-(4-(2-amino-2-carboxyethyl)phenyl)-3 ,12-dioxo-5,8- dioxa-2,11-diazatridecan-13-yl)-1,4,7-triaz onane-1,4-diyl)diaceti c acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 111 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2',2'',2'''-(2-(4-(3-(2-(2-(2-((4-((S)-2-amino-2- carboxyethyl)b enzyl)amino)-2-oxoethoxy)ethoxy)ethyl)thiou reido)benzyl)-1,4,7,10 -tetraazacyclododecane-1,4,7,10-tetrayl)tetraacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 112 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2',2''-(10-((R)-1-(4-((S)-2-amino-2-carboxyethyl) phenyl)-15-car boxy-3,12-dioxo-5,8-dioxa-2,11-diazapentade can-15-yl)-1,4,7,10-te traazacyclododecane-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 113 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2',2'',2'''-(2-(4-(3-(2-(2-(2-((4-((S)-2-amino-2- carboxyethyl)b enzyl)amino)-2-oxoethoxy)ethoxy)ethyl)thiou reido)benzyl)-1,4,7,10 -tetraazacyclododecane-1,4,7,10-tetrayl)tetraacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 114 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2'-(7-(1-(4-((S)-2-amino-2-carboxyethyl)phenyl)-1 5-carboxy-3,12 -dioxo-5,8-dioxa-2,11-diazapentadecan-15-yl )-1,4,7-triazonane-1,4 -diyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 115 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2'-((6-(1-(4-(2-amino-2-carboxyethyl)phenyl)- 3,12-dioxo-5,8 -dioxa-2,11-diazahexadecan-16-yl)-1,4-bis(c arboxymethyl)-1,4-diaz epan-6-yl)azanediyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 116 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2,2'-(7-(1-(4-(2-amino-2-carboxyethyl)phenyl)-3 ,12-dioxo-5,8- dioxa-2,11-diazatridecan-13-yl)-1,4,7-triaz onane-1,4-diyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 117 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 118 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 119 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 120 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
XGXXXWGXDP

SEQ ID NO: 121 - protein, 10 aa, synthetic construct
  REGION 1..10: note = Amino acid sequence
  MOD_RES 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
EGXXXWGXDP

SEQ ID NO: 122 - protein, 10 aa, synthetic construct
  REGION 1..10: note = Amino acid sequence
  REGION 1..10: note = Peptide is circular
  MOD_RES 1: note = L-homoserine
  MOD_RES 3: note = (S)-2-(methylamino)-3-(pyridine-3-yl)propanoic acid
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
XGXXXXGXDP

SEQ ID NO: 123 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
EGXXXXGXDP

SEQ ID NO: 124 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((2-((4-(2-amino-2-car boxyethyl)benzyl)amino) -2-oxoethyl)(methyl)amino)-2-oxoet hyl)-1,4,7,10-tetraazacyclodode cane-1,4,7-triyl)triacetic acid
  MOD_RES 5: note = (S)-2-aminoheptanoic acid
  SITE 10: note = D-Proline
GGXXXWGYDP

SEQ ID NO: 125 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((2-((4-(2-amino-2-car boxyethyl)benzyl)amino) -2-oxoethyl)amino)-2-oxoethyl)-1,4 ,7,10-tetraazacyclododecane-1,4 ,7-triyl)triacetic acid
  MOD_RES 5: note = (S)-2-aminoheptanoic acid
  SITE 10: note = D-Proline
GGXXXWGYDP

SEQ ID NO: 126 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((3-((4-(2-amino-2-car boxyethyl)benzyl)amino) -3-oxopropyl)amino)-2-oxoethyl)-1, 4,7,10-tetraazacyclododecane-1, 4,7-triyl)triacetic acid
  MOD_RES 5: note = (S)-2-aminoheptanoic acid
  SITE 10: note = D-Proline
GGXXXWGYDP

SEQ ID NO: 127 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((3-((4-(2-amino-2-car boxyethyl)benzyl)amino) -3-oxopropyl)(methyl)amino)-2-oxoe thyl)-1,4,7,10-tetraazacyclodod ecane-1,4,7-triyl)triacetic acid
  MOD_RES 5: note = (S)-2-aminoheptanoic acid
  SITE 10: note = D-Proline
GGXXXWGYDP

SEQ ID NO: 128 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
PGXXXWGXDP

SEQ ID NO: 129 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 130 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-(((1-(4-(2-amino-2-car boxyethyl)benzyl)-1H-1, 2,3-triazol-4-yl)methyl)amino)-2-o xoethyl)-1,4,7,10-tetraazacyclo dodecane-1,4,7-triyl)triacetic acid
  MOD_RES 5: note = (S)-2-aminoheptanoic acid
  SITE 10: note = D-Proline
GGXXXWGYDP

SEQ ID NO: 131 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminoheptanoic acid
  SITE 10: note = D-Proline
GGXXXWGYDP

SEQ ID NO: 132 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 5: note = (S)-2-aminoheptanoic acid
  MOD_RES 4: note = lutetium (S)-2,2',2''-(10-(2-((4-(2-amino-2-carboxy ethyl)benzyl)amino)-2-oxoethyl)-1,4,7,10-tetraazacyclodode cane-1,4,7-triyl)triacetic acid
  SITE 10: note = D-Proline
GGXXXWGYDP

SEQ ID NO: 133 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = (S)-2-amino-3-(4-methoxyphenyl)propanoic acid
  SITE 10: note = D-Proline
GGXXXWGXDP

SEQ ID NO: 134 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododecane-1,4,7-triyl)triacetic acid
  MOD_RES 5: note = (S)-2-aminooctanoic acid
  MOD_RES 8: note = (S)-2-amino-3-hydroxyphenyl)propanoic acid
  SITE 10: note = D-Proline
GGXXXWGXDP

SEQ ID NO: 135 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-amino-5-((S)-2-cyano-4,4-difluoropyrrolidiny- 1-yl-5-oxopentanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 136 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 6: note = N6-(4-(carboxymethyl)piperazine-1-carbonyl)-L-lysin e
  SITE 10: note = D-Proline
PGXXXWGXDP

SEQ ID NO: 137 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = O-propyl-L-homoserine
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 138 - protein, 10 aa, synthetic construct
  REGION 1..10: note = Amino acid sequence
  REGION 1..10: note = Peptide is circular
  MOD_RES 1: note = lutetium (S)-2,2',2''-(10-(2-((5-amino-5-carboxypen tyl)amino)-2-oxoethyl)- 1,4,7,10-tetraazacyclododecane-1,4,7-triyl)triacetic acid
  MOD_RES 3: note = methyl-L-phenylalanine
  MOD_RES 5: note = (S)-2-aminoheptanoic acid
  SITE 10: note = D-Proline
XGXYXWGYDP

SEQ ID NO: 139 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((2-(4-(2-amino-2-carb oxyethyl)phenoxy)ethyl) amino)-2-oxoethyl)-1,4,7,10-tetraa zacyclododecane-1,4,7-triyl)tri acetic acid
  MOD_RES 5: note = (S)-2-aminoheptanoic acid
  SITE 10: note = D-Proline
GGXXXWGYDP

SEQ ID NO: 140 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((2-(4-(2-amino-2-carb oxyethyl)phenoxy)ethyl) amino)-2-oxoethyl)-1,4,7,10-tetraa zacyclododecane-1,4,7-triyl)tri acetic acid
  MOD_RES 5: note = (S)-2-aminoheptanoic acid
  MOD_RES 8: note = (S)-2-amino-3-(6-aminopyridin-3-yl)propanoic acid
  SITE 10: note = D-Proline
GGXXXWGXDP

SEQ ID NO: 141 - protein, 10 aa, synthetic construct
  REGION 1..10: note = Amino acid sequence
  REGION 1..10: note = Peptide is circular
  MOD_RES 1: note = N6-(4-(carboxymethyl)piperazine-1-carbonyl)-L-lysin e
  MOD_RES 3: note = methyl-L-phenylalanine
  MOD_RES 4: note = lutetium (S)-2,2',2''-(10-(2-((2-(4-(2-amino-2-carb oxyethyl)phenoxy)ethyl) amino)-2-oxoethyl)-1,4,7,10-tetraa zacyclododecane-1,4,7-triyl)tri acetic acid
  MOD_RES 5: note = (S)-2-aminoheptanoic acid
  SITE 10: note = D-Proline
XGXXXWGYDP

SEQ ID NO: 142 - protein, 10 aa, synthetic construct
  REGION 1..10: note = Amino acid sequence
  REGION 1..10: note = Peptide is circular
  MOD_RES 1: note = L-homoserine
  MOD_RES 3: note = methyl-L-phenylalanine
  MOD_RES 4: note = lutetium (S)-2,2',2''-(10-(2-((2-(4-(2-amino-2-carb oxyethyl)phenoxy)ethyl) amino)-2-oxoethyl)-1,4,7,10-tetraa zacyclododecane-1,4,7-triyl)tri acetic acid
  MOD_RES 5: note = (S)-2-aminoheptanoic acid
  SITE 10: note = D-Proline
XGXXXWGYDP

SEQ ID NO: 143 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(2-((2-(4-(2-amino-2-carb oxyethyl)phenoxy)ethyl) amino)-2-oxoethyl)-1,4,7,10-tetraa zacyclododecane-1,4,7-triyl)tri acetic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 144 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 6: note = (S)-2-amino-3-(1H-pyrrolo[2,3-b]pyridin-3-yl)propan oic acid
  MOD_RES 8: note = N6-(4-(carboxymethyl)piperazine-1-carbonyl)-L-lysin e
  SITE 10: note = D-Proline
GGXXXXGXDP

SEQ ID NO: 145 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 146 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2'-(7-(4-((2-(4-((S)-2-amino-2-carboxyet hyl)phenoxy)ethyl)amino )-1-carboxy-4-oxobutyl)-1,4,7-triazonane-1,4-diyl)diacetic acid
  MOD_RES 5: note = (S)-2-aminoheptanoic acid
  SITE 10: note = D-Proline
GGXXXWGYDP

SEQ ID NO: 147 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2',2''-(10-((R)-4-((2-(4-((S)-2-amino-2- carboxyethyl)phenoxy)et hyl)amino)-1-carboxy-4-oxobutyl)-1 ,4,7,10-tetraazacyclododecane-1 ,4,7-triyl)triacetic acid
  MOD_RES 5: note = (S)-2-aminoheptanoic acid
  SITE 10: note = D-Proline
GGXXXWGYDP

SEQ ID NO: 148 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2'-((6-(1-(4-(2-amino-2-carboxyethyl )phenyl)-3,12-dioxo-5,8 -dioxa-2,11-diazahexadecan-16-yl)- 1,4-bis(carboxymethyl)-1,4-diaz epan-6-yl)azanediyl)diacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 149 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2',2''-(10-((R)-1-(4-((S)-2-amino-2-carb oxyethyl)phenyl)-15-car boxy-3,12-dioxo-5,8-dioxa-2,11-dia zapentadecan-15-yl)-1,4,7,10-te traazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 150 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2',2''-(10-((R)-1-(4-((S)-2-amino-2-carb oxyethyl)phenyl)-15-car boxy-3,12-dioxo-5,8-dioxa-2,11-dia zapentadecan-15-yl)-1,4,7,10-te traazacyclododecane-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 151 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2',2'',2'''-(2-(4-(3-(2-(2-(2-((4-((S)-2 -amino-2-carboxyethyl)b enzyl)amino)-2-oxoethoxy)ethoxy)et hyl)thioureido)benzyl)-1,4,7,10 -tetraazacyclododecane-1,4,7,10-tetrayl)tetraacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 152 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2'-(7-(1-(4-((S)-2-amino-2-carboxyethyl) phenyl)-15-carboxy-3,12 -dioxo-5,8-dioxa-2,11-diazapentade can-15-yl)-1,4,7-triazonane-1,4 -diyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 153 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2'-((6-(1-(4-(2-amino-2-carboxyethyl )phenyl)-3,12-dioxo-5,8 -dioxa-2,11-diazahexadecan-16-yl)- 1,4-bis(carboxymethyl)-1,4-diaz epan-6-yl)azanediyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 154 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium 2,2',2''-(10-((R)-4-((2-(4-((S)-2-amino-2-c arboxyethyl)phenoxy)et hyl)amino)-1-carboxy-4-oxobutyl)-1, 4,7,10-tetraazacyclododecane-1 ,4,7-triyl)triacetic acid
  MOD_RES 5: note = (S)-2-aminoheptanoic acid
  SITE 10: note = D-Proline
GGXXXWGYDP

SEQ ID NO: 155 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium 2,2',2''-(10-((R)-1-(4-((S)-2-amino-2-carbo xyethyl)phenyl)-15-car boxy-3,12-dioxo-5,8-dioxa-2,11-diaz apentadecan-15-yl)-1,4,7,10-te traazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 156 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium 2,2'-(7-(1-(4-((S)-2-amino-2-carboxyethyl)p henyl)-15-carboxy-3,12 -dioxo-5,8-dioxa-2,11-diazapentadec an-15-yl)-1,4,7-triazonane-1,4 -diyl)diacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 157 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2'-(7-(1-(4-(2-amino-2-carboxyethyl)p henyl)-3,12-dioxo-5,8- dioxa-2,11-diazatridecan-13-yl)-1,4 ,7-triazonane-1,4-diyl)diacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 158 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium 2,2'-(7-(1-(4-((S)-2-amino-2-carboxyethyl)p henyl)-15-carboxy-3,12 -dioxo-5,8-dioxa-2,11-diazapentadec an-15-yl)-1,4,7-triazonane-1,4 -diyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 159 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2'-(7-(1-(4-(2-amino-2-carboxyethyl)p henyl)-3,12-dioxo-5,8- dioxa-2,11-diazatridecan-13-yl)-1,4 ,7-triazonane-1,4-diyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 160 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 161 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
EGXXXWGXDP

SEQ ID NO: 162 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 163 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
PGXXXWGXDP

SEQ ID NO: 164 - protein, 10 aa, synthetic construct
  REGION 1..10: note = Amino acid sequence
  REGION 1..10: note = Peptide is circular
  MOD_RES 3: note = (S)-2-(methylamino)-3-(pyridine-3-yl)propanoic acid
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 165 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = (S)-2-aminoheptanoic acid
  SITE 10: note = D-Proline
GGXXXWGYDP

SEQ ID NO: 166 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(2-((2-(4-(2-amino-2-carbo xyethyl)phenoxy)ethyl) amino)-2-oxoethyl)-1,4,7,10-tetraaz acyclododecane-1,4,7-triyl)tri acetic acid
  MOD_RES 5: note = (S)-2-aminoheptanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 167 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(2-((2-(4-(2-amino-2-carbo xyethyl)phenoxy)ethyl) amino)-2-oxoethyl)-1,4,7,10-tetraaz acyclododecane-1,4,7-triyl)tri acetic acid
  MOD_RES 5: note = (S)-2-aminoheptanoic acid
  SITE 10: note = D-Proline
GGXXXWGYDP

SEQ ID NO: 168 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = gallium (S)-2,2',2''-(10-(1-(4-(2-amino-2-carboxyet hyl)phenyl)-3,12-dioxo -5,8-dioxa-2,11-diazatridecan-13-yl )-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 169 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = aluminum [18F]fluoride (S)-2,2'-(7-(1-(4-(2-amino-2 -carboxyethyl)phenyl)-3,12-dioxo-5,8- dioxa-2,11-diazatrid ecan-13-yl)-1,4,7-triazonane-1,4-diyl)diaceti c acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 170 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = aluminum [18F]fluoride (S)-2,2'-(7-(1-(4-(2-amino-2 -carboxyethyl)phenyl)-3,12-dioxo-5,8- dioxa-2,11-diazatrid ecan-13-yl)-1,4,7-triazonane-1,4-diyl)diacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 171 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(12-(6-fluoropyridin-3-yl)-3,12-di oxo-5,8-dioxa- 2,11-diazadodecyl)phenyl)propanoic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 172 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(12-(6-fluoropyridin-3-yl)-3,12-di oxo-5,8-dioxa- 2,11-diazadodecyl)phenyl)propanoic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 173 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-5-((2-(2-(2-((4-(2-amino-2-carboxyethyl)benzyl) amino)-2-oxoet hoxy)ethoxy)ethyl)carbamoyl)-N,N,N-trimethy lpyridin-2-aminium trifluoroacetate
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 174 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-5-((2-(2-(2-((4-(2-amino-2-carboxyethyl)benzyl) amino)-2-oxoet hoxy)ethoxy)ethyl)carbamoyl)-N,N,N-trimethy lpyridin-2-aminium trifluoroacetate
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 175 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = 2-((1E,3E)-5-((E)-1-(6-(((S)-5-amino-5-carboxypenty l)amino)-6-oxo hexyl)-3,3-dimethyl-5-sulfoindolin-2-yliden e)penta-1,3-dien-1-yl) -1-ethyl-3,3-dimethyl-3H-indol-1-ium-5-sulfonate
  SITE 10: note = D-Proline
GGXXXWGXDP

SEQ ID NO: 176 - protein, 10 aa, synthetic construct
  REGION 1..10: note = Amino acid sequence
  REGION 1..10: note = Peptide is circular
  MOD_RES 1: note = 2-((1E,3E)-5-((E)-1-(6-(((S)-5-amino-5-carboxypenty l)amino)-6-oxo hexyl)-3,3-dimethyl-5-sulfoindolin-2-yliden e)penta-1,3-dien-1-yl) -1-ethyl-3,3-dimethyl-3H-indol-1-ium-5-sulfonate
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
XGXXXWGXDP

SEQ ID NO: 177 - protein, 10 aa, synthetic construct
  REGION 1..10: note = Amino acid sequence
  REGION 1..10: note = Peptide is circular
  MOD_RES 2: note = N6-((S)-4-amino-4-carboxybutanoyl)-N2-(4-(4-iodophe nyl)butanoyl)- L-lysine
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GXXXXWGXDP

SEQ ID NO: 178 - protein, 10 aa, synthetic construct
  REGION 1..10: note = Amino acid sequence
  REGION 1..10: note = Peptide is circular
  MOD_RES 2: note = N2-((S)-4-amino-4-carboxybutanoyl)-N6-(5-(4-iodophe nyl)pentanoyl)-L-lysine
  MOD_RES 3: note = methyl-L-phenylalanine
  MOD_RES 4: note = lutetium (S)-2,2',2''-(10-(1-(4-(2-amino-2-carboxye thyl)phenyl)-3,12-dioxo -5,8-dioxa-2,11-diazatridecan-13-y l)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GXXXXWGXDP

SEQ ID NO: 179 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [177Lu]lutetium (S)-2,2',2''-(10-(2-((2-(4-(2-amino -2-carboxyethyl)phenoxy)ethyl) amino)-2-oxoethyl)-1,4,7,10 -tetraazacyclododecane-1,4,7-triyl)tri acetic acid
  MOD_RES 5: note = (S)-2-aminoheptanoic acid
  SITE 10: note = D-Proline
GGXXXWGYDP

SEQ ID NO: 180 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [177Lu]lutetium (S)-2,2',2''-(10-(1-(4-(2-amino-2-c arboxyethyl)phenyl)-3,12-dioxo -5,8-dioxa-2,11-diazatridec an-13-yl)-1,4,7,10-tetraazacyclododeca ne-1,4,7-triyl)triacetic acid
  MOD_RES 5: note = (S)-2-amino-5-((S)-2-cyano-4,4-difluoropyrrolidiny- 1-yl-5-oxopentanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 181 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [177Lu]lutetium (S)-2,2',2''-(10-(1-(4-(2-amino-2-c arboxyethyl)phenyl)-3,12-dioxo -5,8-dioxa-2,11-diazatridec an-13-yl)-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 182 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [177Lu]lutetium (S)-2,2',2''-(10-(1-(4-(2-amino-2-c arboxyethyl)phenyl)-3,12-dioxo -5,8-dioxa-2,11-diazatridec an-13-yl)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 183 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [68Ga]gallium (S)-2,2',2''-(10-(1-(4-(2-amino-2-car boxyethyl)phenyl)-3,12-dioxo -5,8-dioxa-2,11-diazatridecan -13-yl)-1,4,7,10-tetraazacyclododeca ne-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 184 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [68Ga]gallium (S)-2,2',2''-(10-(1-(4-(2-amino-2-car boxyethyl)phenyl)-3,12-dioxo -5,8-dioxa-2,11-diazatridecan -13-yl)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 185 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = (S)-2-amino-3-(4-(12-(6-(18F)fluoropyridin-3-yl)-3, 12-dioxo-5,8-d ioxa-2,11-diazadodecyl)phenyl)propanoic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 186 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = [225Ac]actinium (S)-2,2',2''-(10-(1-(4-(2-amino-2-c arboxyethyl)phenyl)-3,12-dioxo -5,8-dioxa-2,11-diazatridec an-13-yl)-1,4,7,10-tetraazacyclododeca ne-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 187 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = 2,2',2''-(10-(1-(4-((S)-2-amino-2-carboxyethyl)phen yl)-14-(1-(4-( (S)-2-amino-2-carboxyethyl)phenyl)-3,12-dio xo-5,8-dioxa-2,11-diaz atridecan-13-yl)-3,12,16-trioxo-5,8 -dioxa-2,11,15-triazaheptadeca n-17-yl)-1,4,7,10-tetraazac yclododecane-1,4,7-triyl)triaceticacid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 188 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2',2''-(10-(1-(4-((S)-2-amino-2-carboxye thyl)phenyl)-14-(1-(4-((S)-2-amino-2-carboxyethyl)phenyl)- 3,12-dioxo-5,8-dioxa-2,11-diazatridecan-13-yl)-3,12,16-tri oxo-5,8-dioxa-2,11,15-triazaheptadecan-17-yl)-1,4,7,10-tet raazacyclododecane-1,4,7-triyl)triacetic acid
  MOD_RES 5: note = (S)-2-aminooctanoic acid
  MOD_RES 8: note = N6-(4-(carboxymethyl)piperazine-1-carbonyl)-L-lysin e
  SITE 10: note = D-Proline
GGXXXWGXDP

SEQ ID NO: 189 - protein, 10 aa, synthetic construct
  REGION 1..10: note = Amino acid sequence
  REGION 1..10: note = Peptide is circular
  MOD_RES 3: note = methyl-L-phenylalanine
  MOD_RES 4: note = lutetium 2,2',2''-(10-(1-(4-((S)-2-amino-2-carboxye thyl)phenyl)-14-(1-(4-((S)-2-amino-2-carboxyethyl)phenyl)- 3,12-dioxo-5,8-dioxa-2,11-diazatridecan-13-yl)-3,12,16-tri oxo-5,8-dioxa-2,11,15-triazaheptadecan-17-yl)-1,4,7,10-tet raazacyclododecane-1,4,7-triyl)triacetic acid
  MOD_RES 5: note = O-propyl-L-homoserine
  MOD_RES 8: note = N6-(4-(carboxymethyl)piperazine-1-carbonyl)-L-lysin e
  SITE 10: note = D-Proline
GGXXXWGXDP

SEQ ID NO: 190 - protein, 10 aa, synthetic construct
  REGION 1..10: note = Amino acid sequence
  REGION 1..10: note = Peptide is circular
  MOD_RES 1: note = N6-(4-(carboxymethyl)piperazine-1-carbonyl)-L-lysin e
  MOD_RES 3: note = methyl-L-phenylalanine
  MOD_RES 4: note = 2,2',2''-(10-(1-(4-((S)-2-amino-2-carboxyethyl)phen oxy)-18-(17-(4-((S)-2-amino-2-carboxyethyl)phenoxy)-2,14-d ioxo-6,9,12-trioxa-3,15-diazaheptadecyl)-4,16,20-trioxo-6, 9,12-trioxa-3,15,19-triazahenicosan-21-yl)-1,4,7,10-tetraa zacyclododecane-1,4,7-triyl)triacetic acid
  MOD_RES 5: note = (S)-2-aminoheptanoic acid
  SITE 10: note = D-Proline
XGXXXWGYDP

SEQ ID NO: 191 - protein, 10 aa, synthetic construct
  REGION 1..10: note = Amino acid sequence
  MOD_RES 1: note = N6-(4-(carboxymethyl)piperazine-1-carbonyl)-L-lysin e
  MOD_RES 3: note = methyl-L-phenylalanine
  MOD_RES 4: note = lutetium 2,2',2''-(10-(1-(4-((S)-2-amino-2-carboxye thyl)phenyl)-14-(1-(4-((S)-2-amino-2-carboxyethyl)phenyl)- 3,12-dioxo-5,8-dioxa-2,11-diazatridecan-13-yl)-3,12,16-tri oxo-5,8-dioxa-2,11,15-triazaheptadecan-17-yl)-1,4,7,10-tet raazacyclododecane-1,4,7-triyl)triacetic acid
  MOD_RES 5: note = (S)-2-aminoheptanoic acid
  SITE 10: note = D-Proline
  REGION 1..10: note = Peptide is circular
XGXXXWGYDP

SEQ ID NO: 192 - protein, 10 aa, synthetic construct
  REGION 1..10: note = Amino acid sequence
  SITE 1: note = D-Serine
  REGION 1..10: note = Peptide is circular
  SITE 10: note = D-Proline
  VARIANT 1: note = X can be any amino acid (D,G,MeGly,E,K, 2-((1E,3E)- 5-((E)-1-(6-(((S)-5-amino-5-carboxypentyl)amino)-6-oxohexy l)-3,3-dimethyl-5-sulfoindolin-2-ylidene)penta-1,3-dien-1- yl)-1-ethyl-3,3-dimethyl-3H-indol-1-ium-5-sulfonate,N6-(4- (carboxymethyl)piperazine-1-carbonyl)-L-lysine,S,D-Serine, L-homoserine,P or (2S,4R)-4-hydroxypyrrolidine-2-carboxylic acid)
  MOD_RES 1: note = MeGly, 2-((1E,3E)-5-((E)-1-(6-(((S)-5-amino-5-carbo xypentyl)amino)-6-oxohexyl)-3,3-dimethyl-5-sulfoindolin-2- ylidene)penta-1,3-dien-1-yl)-1-ethyl-3,3-dimethyl-3H-indol -1-ium-5-sulfonate,N6-(4-(carboxymethyl)piperazine-1-carbo nyl)-L-lysine,L-homoserine or (2S,4R)-4-hydroxypyrrolidine-2-carboxylic acid
  VARIANT 3: note = X can be F or methyl-L-phenylalanine
  MOD_RES 3: note = methyl-L-phenylalanine
  VARIANT 5: note = X can be (S)-2-aminoheptanoic acid, (S)-2-aminooctanoic acid, O-propyl-L-homoserine or (S)-2-amino-5-((S)-2-cyano-4,4-difluoropyrrolidiny-1-yl-5- oxopentanoic acid
  MOD_RES 5: note = (S)-2-aminoheptanoic acid, (S)-2-aminooctanoic acid, O-propyl-L-homoserine or (S)-2-amino-5-((S)-2-cyano- 4,4-difluoropyrrolidiny-1-yl-5-oxopentanoic acid
  VARIANT 6: note = X can be W,1-(tert-butoxycarbonyl)-L-tryptophan,(S) -2-amino-3-(6-hydroxy-1H-indol-3-yl)propanoic acid, (S)-2-amino-3-(7-chloro-1H-indol-3-yl)propanoic acid, (S)-2-amino-3-(7-fluoro-1H-indol-3-yl)propanoic acid, (S)-2-amino-3-(1H-pyrrolo[2,3-b]pyridin-3-yl)propanoic acid, or (S)-2-amino-3-(7-methoxy-1H-indol-3-yl)propanoic acid
  MOD_RES 6: note = 1-(tert-butoxycarbonyl)-L-tryptophan,(S)-2-amino-3- (6-hydroxy-1H-indol-3-yl)propanoic acid, (S)-2-amino-3-(7-chloro-1H-indol-3-yl)propanoic acid, (S)-2-amino-3-(7-fluoro-1H-indol-3-yl)propanoic acid, (S)-2-amino-3-(1H-pyrrolo[2,3-b]pyridin-3-yl)propanoic acid, or (S)-2-amino-3-(7-methoxy-1H-indol-3-yl)propanoic acid
  VARIANT 8: note = X can be any amino acid( Y or N6-(4-(carboxymethyl)piperazine-1-carbonyl)-L-lysine)
  MOD_RES 8: note = N6-(4-(carboxymethyl)piperazine-1-carbonyl)-L-lysin e
XGXFXXGXDP

SEQ ID NO: 193 - protein, 10 aa, synthetic construct
  REGION 1..10: note = Amino acid sequence
  REGION 1..10: note = Peptide is circular
  SITE 10: note = D-Proline
  VARIANT 3: note = X can be F or methyl-L-phenylalanine
  MOD_RES 3: note = methyl-L-phenylalanine
  MOD_RES 5: note = (S)-2-aminoheptanoic acid, (S)-2-aminooctanoic acid, O-propyl-L-homoserine or (S)-2-amino-5-((S)-2-cyano- 4,4-difluoropyrrolidiny-1-yl-5-oxopentanoic acid
  VARIANT 5: note = X can be (S)-2-aminoheptanoic acid, (S)-2-aminooctanoic acid, O-propyl-L-homoserine or (S)-2-amino-5-((S)-2-cyano-4,4-difluoropyrrolidiny-1-yl-5- oxopentanoic acid
  VARIANT 6: note = X can be W,1-(tert-butoxycarbonyl)-L-tryptophan,(S) -2-amino-3-(6-hydroxy-1H-indol-3-yl)propanoic acid, (S)-2-amino-3-(7-chloro-1H-indol-3-yl)propanoic acid, (S)-2-amino-3-(7-fluoro-1H-indol-3-yl)propanoic acid, (S)-2-amino-3-(1H-pyrrolo[2,3-b]pyridin-3-yl)propanoic acid, or (S)-2-amino-3-(7-methoxy-1H-indol-3-yl)propanoic acid
  MOD_RES 6: note = 1-(tert-butoxycarbonyl)-L-tryptophan,(S)-2-amino-3- (6-hydroxy-1H-indol-3-yl)propanoic acid, (S)-2-amino-3-(7-chloro-1H-indol-3-yl)propanoic acid, (S)-2-amino-3-(7-fluoro-1H-indol-3-yl)propanoic acid, (S)-2-amino-3-(1H-pyrrolo[2,3-b]pyridin-3-yl)propanoic acid, or (S)-2-amino-3-(7-methoxy-1H-indol-3-yl)propanoic acid
  VARIANT 8: note = X can be any amino acid ( Y or N6-(4-(carboxymethyl)piperazine-1-carbonyl)-L-lysine)
  MOD_RES 8: note = N6-(4-(carboxymethyl)piperazine-1-carbonyl)-L-lysin e
GGXFXXGXDP

(19) United States
(12) Patent Application Publication
(10) Pub. No.: US 2025/0090699 A1
(43) Pub. Date: Mar. 20, 2025

(54) FAP TARGETING CYCLIC PEPTIDES AND CONJUGATES THEREOF

(71) Applicant: Novartis AG, Basel (CH)

(72) Inventors: Salvatore BONGARZONE, Loranze (IT); Davide CAMPORESE, Saint Genis Pouilly (FR); Gonçalo dos Santos CLEMENTE, Basel Stadt (CH); Takeru EHARA, Tokyo (JP); Matteo FISCHER, Basel (CH); Andrei Alexandrovich GOLOSOV, Medford, MA (US); Philipp GROSCHE, Inzlingen (DE); Philipp HOLZER, Sissach (CH); Alexei KARPOV, Basel (CH); Marion LACAUD-BAUMLIN, Geispitzen (FR); Lukas LEDER, Biel-Benken (CH); Rafael Bermeo MALO, Basel (CH); Andreas MARZINZIK, Weil am Rhein (DE); Yoshihide MIZUKOSHI, Kanagawa-shi (JP); Charlie MUNSCH, Blotzheim (FR); Markus Oliver RESCHKE, Birsfelden (CH); Holger SELLNER, Buchs (CH); Therese-Marie STACHYRA, Mulhouse (FR)

(21) Appl. No.: 18/802,972

(22) Filed: Aug. 13, 2024

Related U.S. Application Data
(60) Provisional application No. 63/519,599, filed on Aug. 15, 2023.
(60) Provisional application No. 63/650,139, filed on May 21, 2024.

(51) Int. Cl. A61K 51/08; A61P 35/00; C07B 59/00

(52) U.S. Cl. CPC A61K 51/088; A61P 35/00; C07B 59/008; A61K 2121/00; A61K 2123/00; C07B 2200/05

(57) ABSTRACT

Described herein are cyclic peptides targeting fibroblast activation protein (FAP), and their incorporation into compounds for radioligand imaging and therapies, as well as methods and/or uses of such compounds for the imaging, treatment and/or prevention of FAP-implicated diseases and disorders.

### CROSS-REFERENCE TO RELATED APPLICATIONS

This application is related to U.S. Provisional Application Ser. No. 63/519,599 filed Aug. 15, 2023 and U.S. Provisional Application Ser. No. 63/650,139 filed May 21, 2024, which are each hereby incorporated herein by reference in their entirety.

### SEQUENCE LISTING

The instant application contains a Sequence Listing which has been submitted herewith in ST.26 XML format and is hereby incorporated by reference in its entirety. Said ST.26 XML copy, created on Aug. 13, 2024, is named PAT059481-US-NP_ST26_SEQUENCE_LISTING, and is 681,545 bytes in size.

### FIELD

This disclosure relates to cyclic peptides targeting fibroblast activation protein (FAP), and their incorporation into compounds for radioligand imaging and therapies, as well as methods and/or uses of such compounds for the imaging, treatment and/or prevention of FAP-implicated diseases and disorders.

### BACKGROUND

Fibroblasts are present in almost all tissues and usually rest in a quiescent state. Following an injury to tissue integrity, fibroblasts become active, migrate to the site of the injury, and orchestrate damage repair. After the repair, the fibroblasts go back to their quiescent state, however, in the case of chronic inflammation or fibrosis, the fibroblasts remain in an activated state (Lindner et al. 2021, Radioligands Targeting Fibroblast Activation Protein (FAP). Cancers p. 5744). Fibroblast activation protein (FAP) is highly expressed on the surface of activated fibroblasts, such as, for example, cancer-associated fibroblasts (CAFs). CAFs are a major constituent of tumor stroma and play a significant role in the tumor microenvironment as stromal components that affect tumor behavior (Calais 2020, FAP: The Next Billion Dollar Nuclear Theranostics Target? J Nucl Med p. 163). Through the production of growth factors and cytokines, remodeling of the extracellular membrane, and promotion of angiogenesis, CAFs facilitate malignant cell invasion and migration. In addition to these well-known processes of CAFs, it is also believed that they contribute to therapeutic resistance and tumor recurrence (Zou et al. 2022, Pan-cancer analyses and molecular subtypes based on the cancer-associated fibroblast landscape and tumor microenvironment infiltration characterization reveal clinical outcome and immunotherapy response in epithelial ovarian cancer. Front Immunol).

FAP-positive CAFs are found in over 90% of epithelial cancers including, but not limited to, malignant breast, colorectal, lung, skin, prostate, and pancreatic cancers. The prevalence of FAP in tumor stroma, combined with its limited expression in non-damaged tissues, presents a potential for noninvasive tumor characterization, examination, and therapy using FAP-targeting compounds. Although FAP-targeting tracers have been demonstrated to detect different tumor entities with high specificity (for imaging/diagnostics), there remains a need for FAP-targeting therapies that can provide effective treatment with the same high specificity. To that aim, the compounds disclosed herein have high affinity for FAP, have high tumor uptake and retention times, and favorable safety profiles, which make them particularly suitable for therapeutic purposes.

### SUMMARY

Provided herein are cyclic peptides that target fibroblast activation protein (FAP), compounds incorporating such cyclic peptides, which are suitable for radiolabeling, corresponding pharmaceutical compositions, and methods and/or uses of the FAP-targeting compounds (also referred to as FAP-targeting ligands, e.g., FAP-targeting radioligands) for the imaging and treatment of FAP-implicated cancers.

In particular, the present disclosure provides compounds, or pharmaceutically acceptable salts, solvates, stereoisomers, or tautomers thereof, comprising:
a) at least one cyclized peptide

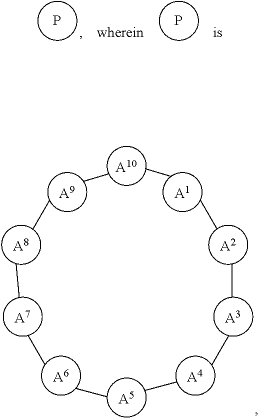

wherein A¹-A¹⁰ are as defined herein; and
b) at least one imaging agent, chelating agent, radionuclide, or cytotoxic drug, wherein the cyclized peptide

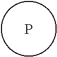

is conjugated to the at least one imaging agent, chelating agent, radionuclide, or cytotoxic drug via any one of A¹-A¹⁰, optionally through a linker.

In some embodiments, the compounds are FAP-targeting compounds of formula (I), (Ia), (Ib), or (Ic):

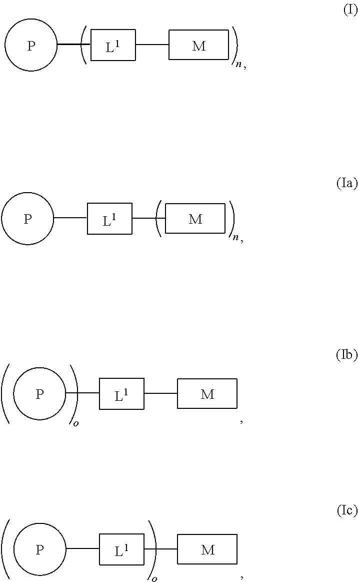

or a pharmaceutically acceptable salt, solvate, stereoisomer, or tautomer thereof,
wherein
L¹ is, independently at each occurrence, a bond or a linker;
M is, independently at each occurrence, an imaging agent, a chelating agent, or a radionuclide,
wherein the chelating agent is optionally radiolabeled with a radionuclide;
n is 1, 2, 3, or 4; and
is 1, 2, 3, or 4,
wherein any of P, L¹, or M is optionally substituted with an albumin binder.

In certain embodiments, the FAP-targeting compounds are radiolabeled with a diagnostic or therapeutic radionuclide. Such radiolabeled compounds can be referred to a FAP-targeting radioligands, FAP-targeting radiotherapeutics, or FAP-targeting radioimaging agents.

The present disclosure further provides pharmaceutical compositions comprising the FAP-targeting compounds described herein, or a pharmaceutically acceptable salt, solvate, stereoisomer, or tautomer thereof, and a pharmaceutically acceptable carrier.

The present disclosure further provides a combination comprising the FAP-targeting compounds described herein, or a pharmaceutically acceptable salt, solvate, stereoisomer, or tautomer thereof, and one or more therapeutically active agents.

The present disclosure further provides a method of imaging FAP-related diseases and disorders, comprising administering to a subject in need thereof a diagnostically effective amount of a FAP-targeting ligand and/or a FAP-targeting radioligand, or a pharmaceutically acceptable salt, solvate, stereoisomer, or tautomer thereof, or a pharmaceutical composition described herein.

The present disclosure further provides a method of treating and/or preventing FAP-related diseases and disorders, comprising administering to a subject in need thereof a therapeutically effective amount of a FAP-targeting ligand and/or a FAP-targeting radioligand, or a pharmaceutically acceptable salt salts, solvate, stereoisomer, or tautomer thereof, or a pharmaceutical composition described herein.

### BRIEF DESCRIPTION OF THE DRAWINGS

FIG. 1 is a graph showing the tumor, kidney, and blood uptake of Example G1 in mice after intravenous injection.

FIG. 2 is a graph showing the tumor, kidney, and blood uptake of Example G2 in mice after intravenous injection.

FIG. 3 is a graph showing the tumor, kidney, and blood uptake of Example G3 in mice after intravenous injection.

FIG. 4 is a graph showing the tumor, kidney, and blood uptake of Example G4 in mice after intravenous injection.

FIG. 5 is a graph showing the antitumor efficacy of Examples G1 and G4 in mice up to 130 days after intravenous injection.

FIG. 6 is a graph showing the antitumor efficacy of Examples G3 and G4 in mice measured up to 130 days after intravenous injection.

FIG. 7 is a graph showing the antitumor efficacy of Example G4 in mice measured up to 84 days after intravenous injection.

### DETAILED DESCRIPTION

FAP is a type II integral membrane serine protease which is strongly expressed by stromal fibroblasts in many carcinomas and sarcomas. Accordingly, FAP-targeting ligands selectively accumulate in different types of cancer, allowing multiple development opportunities for imaging, diagnosing, and treating cancer. Currently, FAP-targeting ligands are predominantly used in imaging applications, however, the short tumor retention time of many of these ligands limit their therapeutic applications. The peptide-based FAP-targeting ligands of the present disclosure herein have high affinity for FAP, have high tumor uptake and retention times, and favorable safety profiles, which make them particularly suitable for therapeutic purposes.

Accordingly, described herein are FAP-targeting compounds (or alternatively, FAP-targeting ligands) comprising:
a) at least one cyclized peptide

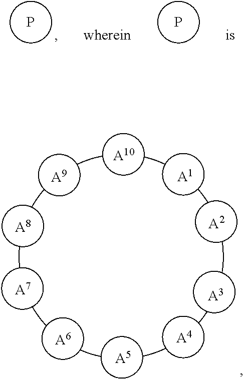

wherein A¹-A¹⁰ are as defined herein; and
b) at least one imaging agent, chelating agent, radionuclide, or cytotoxic drug, wherein the cyclized peptide

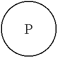

is conjugated to the at least one imaging agent, chelating agent, radionuclide, or cytotoxic drug via any one of A¹-A¹⁰, optionally through a linker.

The FAP-targeting compounds can be, inter alia, a compound of formula (I), (Ia), (Ib), (Ic), or (Id):

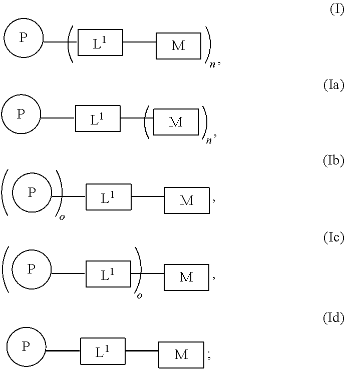

or a pharmaceutically acceptable salt, solvate, stereoisomer, or tautomer thereof,
where P is a cyclic FAP-targeting peptide; M is, independently at each occurrence, selected from the group consisting of an imaging agent, a chelating agent optionally chelated to a radionuclide, a radionuclide, and a cytotoxic drug; L¹ is a bond or a linker adapted to form a chemical bond between P and M; and n and o are each independently 1, 2, 3, or 4.

The compounds disclosed herein, including the compounds of Formula (I), (Ia), (Ib), (Ic), or (Id), or a pharmaceutically acceptable salt, solvate, stereoisomer, or tautomer thereof, exhibit strong binding to FAP, i.e., exhibit a dissociation constant (K_{D}) for human FAP of about 1 nM or less as measured by surface plasmon resonance (SPR) at a temperature of 25° C.

Compounds of Formula (I), (Ia), (Ib), (Ic), or (Id), or a pharmaceutically acceptable salt, solvate, stereoisomer, or tautomer thereof, also exhibit prolonged tumor retention time compared to other FAP-targeting compounds. Accordingly, described herein are methods of targeting FAP, imaging FAP-expression, and treating FAP-related disease, with a compound of any of Formulae (I), (Ia), (Ib), (Ic), or (Id) or a pharmaceutically acceptable salt, solvate, stereoisomer, or tautomer thereof.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

It is further appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable sub-combination.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Remington's Pharmaceutical Sciences,” Mack Publishing Co., Easton, Pa., 21st edition, 2005, which is hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

The terms “peptide,” “polypeptide,” and “protein” are used interchangeably herein and typically refer to a molecule comprising a chain of two or more amino acids (e.g., L-amino acids, D-amino acids, modified amino acids, amino acid analogs, amino acid mimetics, etc.).

Unless otherwise indicated, naturally occurring L-amino acids and D-amino acids are both represented by either conventional three-letter, or capitalized one-letter, amino acid designations of Table 1. In some embodiments, naturally occurring L-amino acids are represented by either conventional three-letter, or capitalized one-letter, amino acid designations of Table 1. In some embodiments, D-amino acids, are represented by lower-case one-letter amino acid designations corresponding to one-letter designations of Table 1, i.e., g, a, 1, m, f, w, k, q, e, s, p, v, i, c, y, h, r, n, d, and t.

TABLE 1
Naturally occurring amino acids

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

The term “L-amino acid,” as used herein, refers to the “L” isomeric form of an amino acid, and conversely the term “D-amino acid” refers to the “D” isomeric form of an amino acid (e.g., (D)Asp or D-Asp; (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, D-arginine can be represented as “arg” or “r.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g., sarcosine, ornithine, etc.), frequently employed three- or four-character codes are employed for residues thereof, including, Sar or Sarc (sarcosine, i.e., N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and Abu (2-aminobutyric acid).

Further, non-limiting examples of non-naturally occurring amino acids, that may appear, e.g., in the compounds of disclosed herein including those of Formulae (I), (Ia), (Ib), (Ic) or (Id) appear in Table 2 below.

TABLE 2
Non-limiting list of non-naturally occurring amino acids that may be incorporated into the
FAP targeting cyclic peptides of the present disclosure:

| | | Examples of incorporation into a |
|---|---|---|
| AA abbreviation | AA structure | cyclic peptide |
| NMePhe | 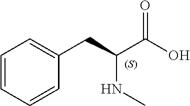 | 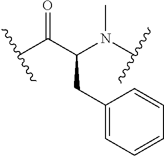 [SMILES: *C(=O)[C@H](Cc1ccccc1)N(*)C] |
| NMecHexA | 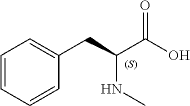 | 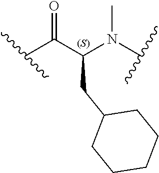 |
| NMecPenA | 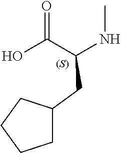 | 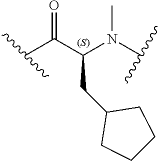 |
| cBuA | 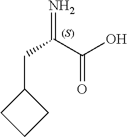 | 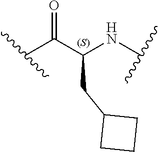 |
| NMecBuA | 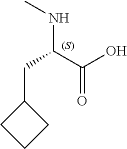 | 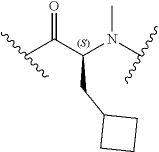 |
| NMe3PyA | 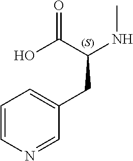 | 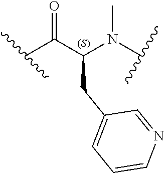 |
| 4PyA | 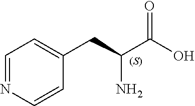 | 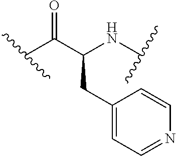 [SMILES: *N[C@@H](Cc1ccncc1)C(*)=O] |
| F(4OMe) | 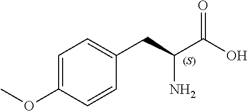 | 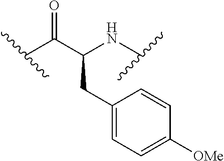 [SMILES: *N[C@@H](Cc1ccc(OC)cc1)C(*)=O] |
| F(4OEt) | 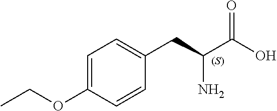 | 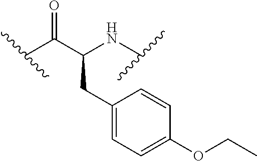 [SMILES: *N[C@@H](Cc1ccc(OCC)cc1)C(*)=O] |
| F(3OH) | 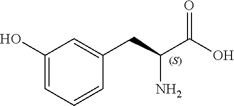 | 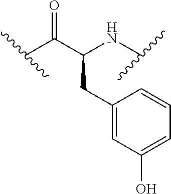 [SMILES: *N[C@@H](Cc1cccc(O)c1)C(*)=O] |
| NMeGly | 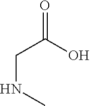 | 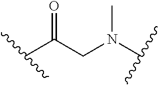 [SMILES: *C(=O)CN(*)C] |
| NMeY | 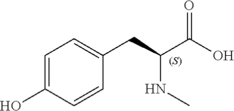 | 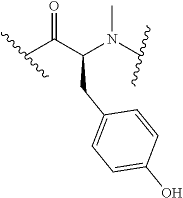 [SMILES: *C(=O)[C@H](Cc1ccc(O)cc1)N(*)C] |
| D-NMeA | 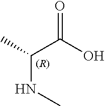 | 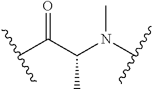 [SMILES: *C(=O)[C@@H](C)N(*)C] |
| D-Ala | 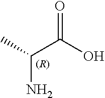 | 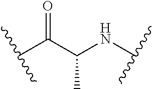 [SMILES: *N[C@H](C)C(*)=O] |
| D-Nle | 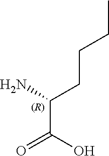 | 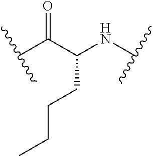 [SMILES: *N[C@H](CCCC)C(*)=O] |
| NBnG | 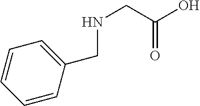 [SMILES: O=C(O)CNCc1ccccc1] | 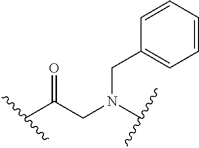 [SMILES: *C(=O)CN(*)Cc1ccccc1] |
| aMeD | 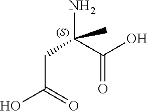 | 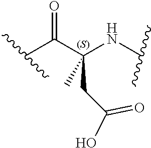 |
| Ahp | 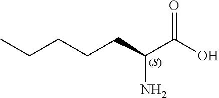 | 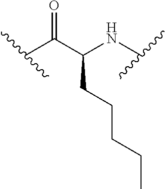 [SMILES: *N[C@@H](CCCCC)C(*)=O] |
| Aoc | 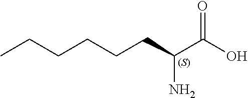 | 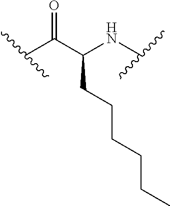 [SMILES: *N[C@@H](CCCCCC)C(*)=O] |
| Aoc(3R-OH) | 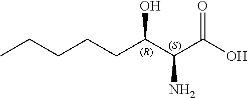 | 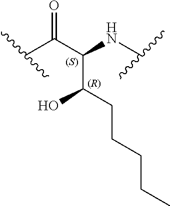 |
| hS(Pr) | 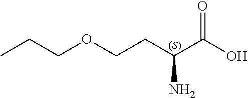 | 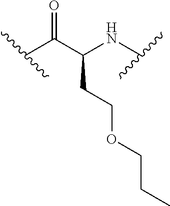 [SMILES: *N[C@@H](CCOCCC)C(*)=O] |
| D-Pro | 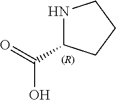 | 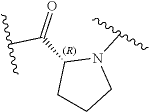 |
| D-trans4hyp | 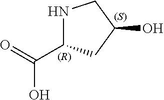 | 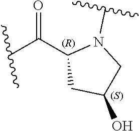 |
| trans4Hyp | 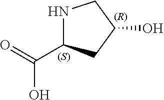 | 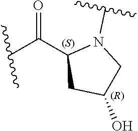 |
| Pi(2R-COOH) | 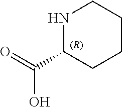 | 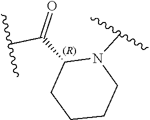 |
| Mor(3R-COOH) | 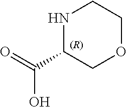 | 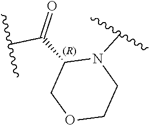 |
| D-aMeP | 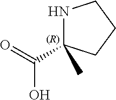 | 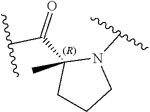 |
| hSer | 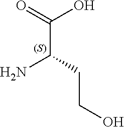 | 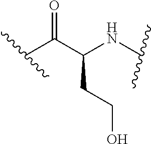 [SMILES: *N[C@@H](CCO)C(*)=O] |
| K(Ac) | 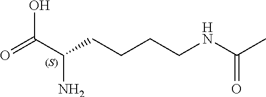 | 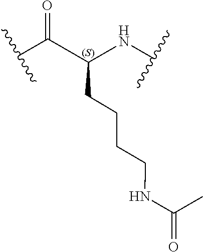 |
| D-K(Ac) | 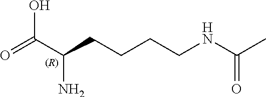 | 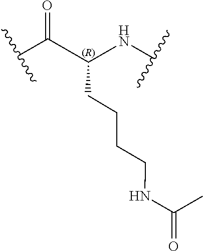 |
| W(6OH) | 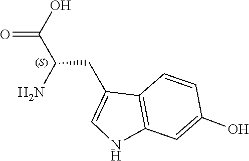 | 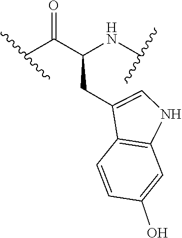 [SMILES: *N[C@@H](Cc1c[nH]c2cc(O)ccc12)C(*)=O] |
| W(7F) | 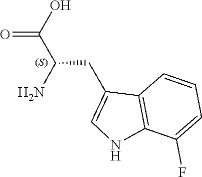 | 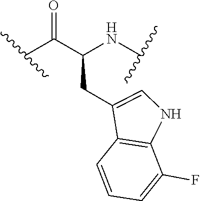 [SMILES: *N[C@@H](Cc1c[nH]c2c(F)cccc12)C(*)=O] |
| W7N | 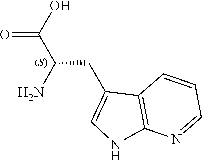 | 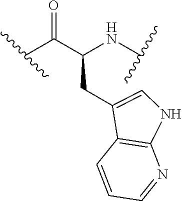 [SMILES: *N[C@@H](Cc1c[nH]c2ncccc12)C(*)=O] |
| W(7Cl) | 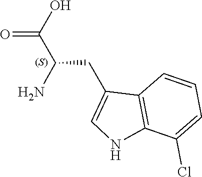 | 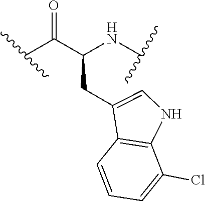 [SMILES: *N[C@@H](Cc1c[nH]c2c(Cl)cccc12)C(*)=O] |
| W(7OMe) | 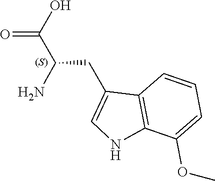 | 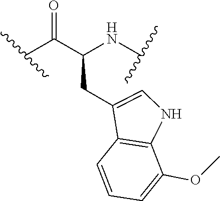 [SMILES: *N[C@@H](Cc1c[nH]c2c(OC)cccc12)C(*)=O] |
| F(4AmAc) | 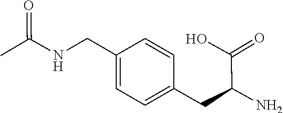 [SMILES: CC(=O)NCc1ccc(C[C@H](N)C(=O)O)cc1] | 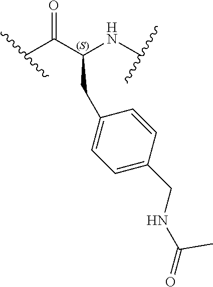 |
| F(4OEtNH2) | 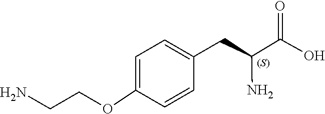 | 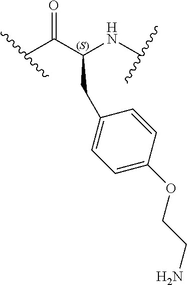 |
| F(4Am) | 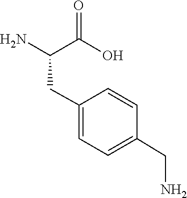 [SMILES: NCc1ccc(C[C@H](N)C(=O)O)cc1] | 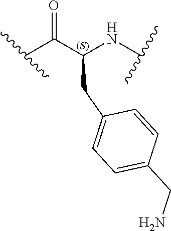 |
| F(4amPEG2NH2) | 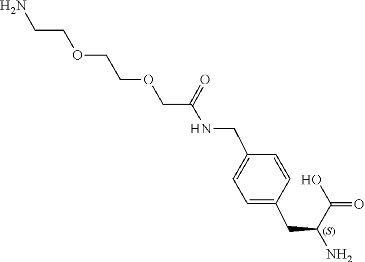 | 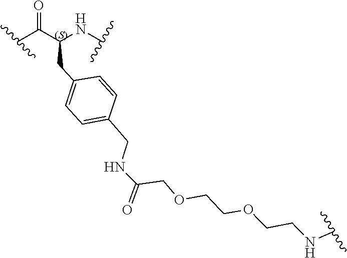 |
| F4(MeTriazolylMe NH2) | 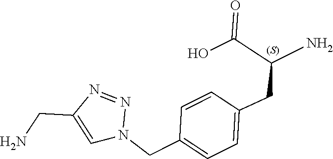 | 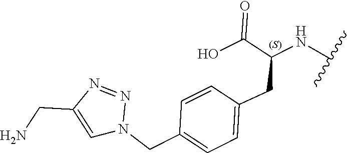 |
| Lys(Cy5) | 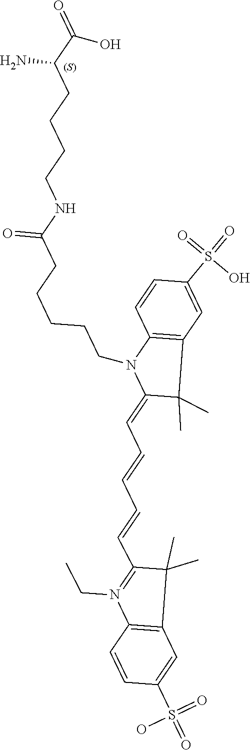 | 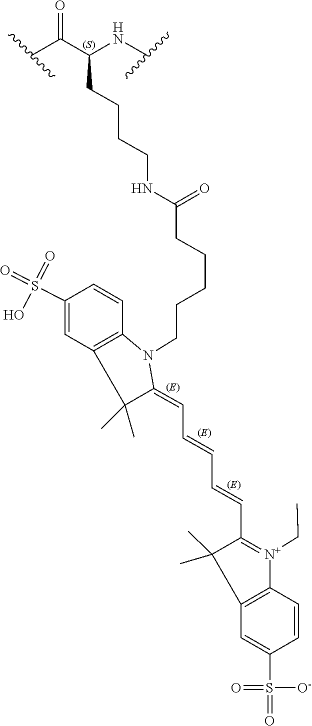 |
| Lys(Dota) | 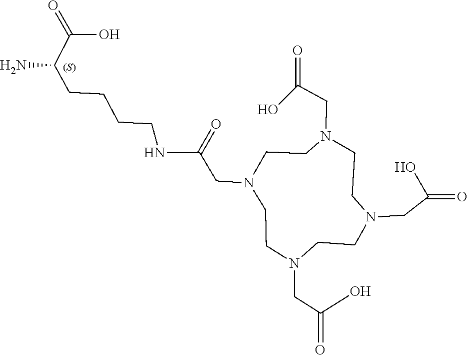 | |
| D-Lys(Dota) | 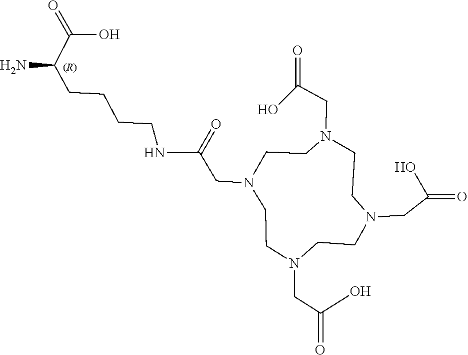 | |
| F(4OEtNHDOTA) | 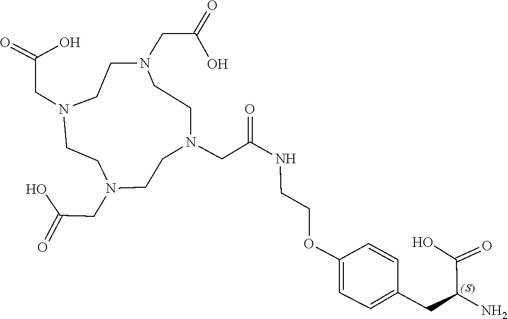 | |
| F(4OEtNH NODAGA) | 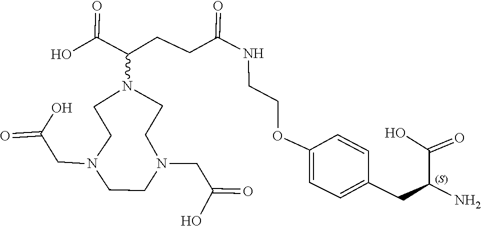 | |
| F(4OEtNH RDOTAGA) | 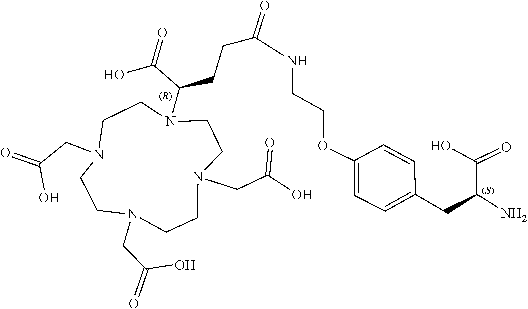 | |
| F(4amDOTA) | 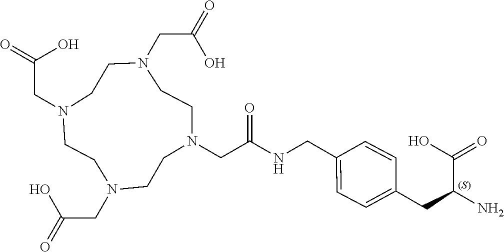 | |
| F(4amPEG2NH DOTA) | 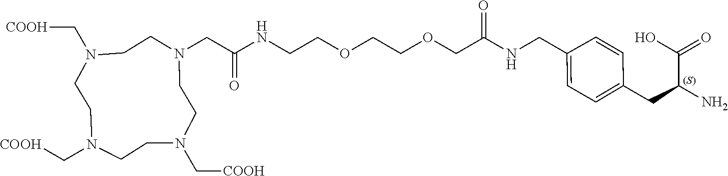 | |
| F(4amPEG2NH AAZTA) | 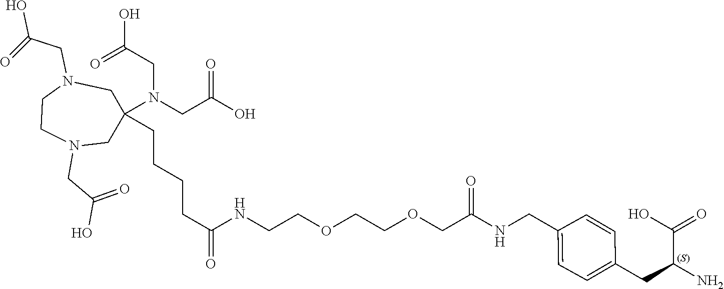 | |
| F(4amPEG2NH NODAGA) | 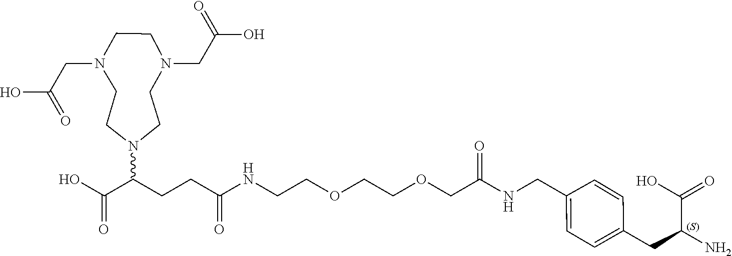 | |
| F(4amPEG2NH RDOTAGA) | 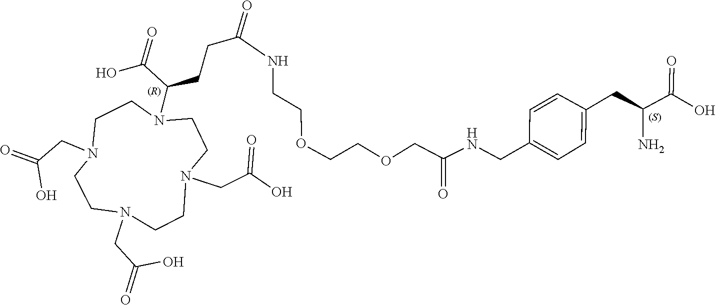 | |
| F(4amPEG2NH NOTA) | 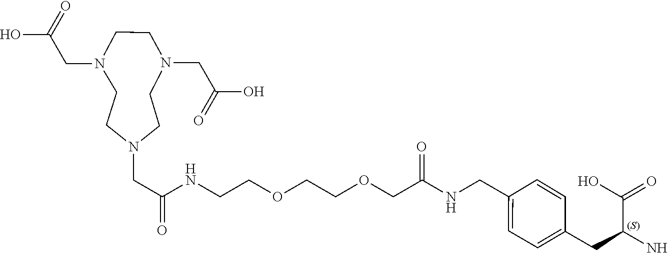 | |
| F(4amGlyDOTA) | 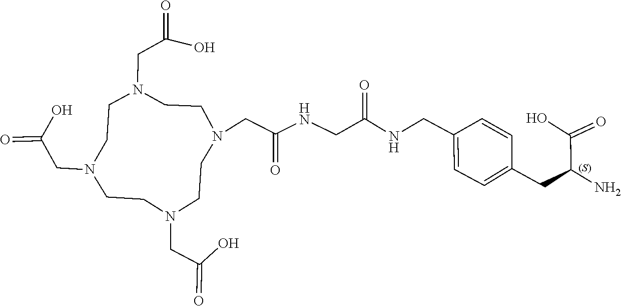 | |
| F(4amMeGly DOTA) | 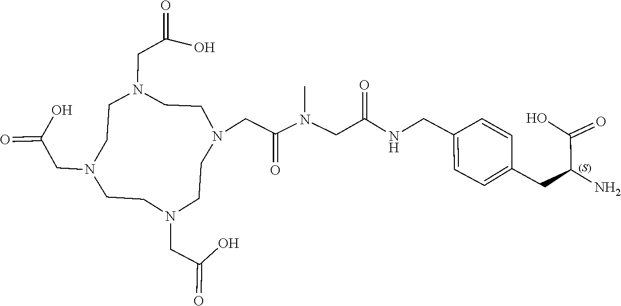 | |
| F(4amBAlaDOTA) | 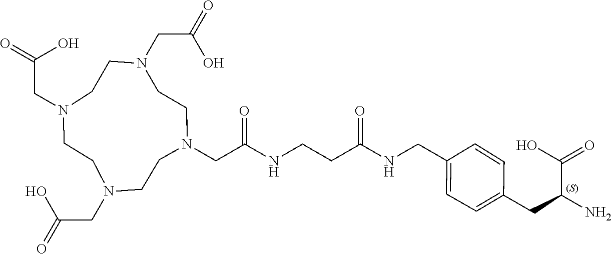 | |
| F(4amMeBAla DOTA) | 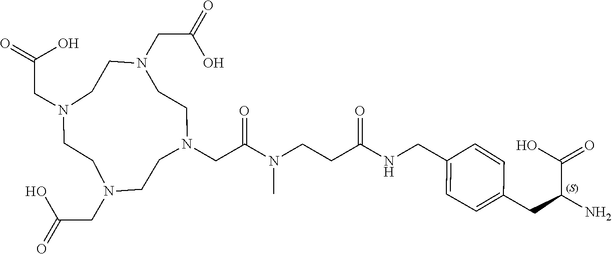 | |
| F(4MeTriazolylMe NHDOTA) | 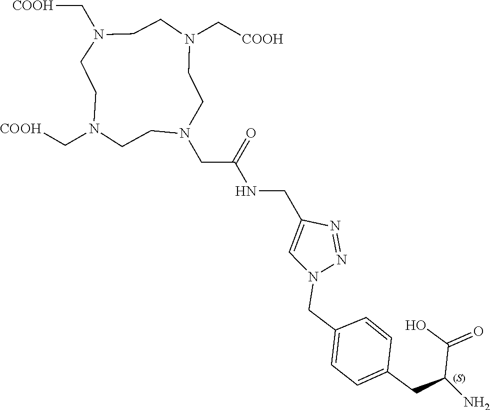 | |
| F(4amPEG2NH- p-SCN-DOTA) | 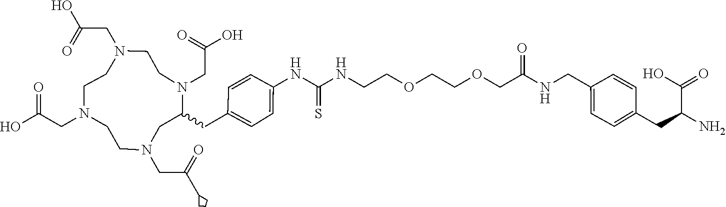 [SMILES: N[C@@H](Cc1ccc(CNC(=O)COCCOCCNC(=S)Nc2ccc(CC3CN(CC(=O)C4CCC4)CCN(CC(=O)O)CCN(CC(=O)O)CCN3CC(=O)O)cc2)cc1)C(=O)O] | |
| K(COpipzaa) | 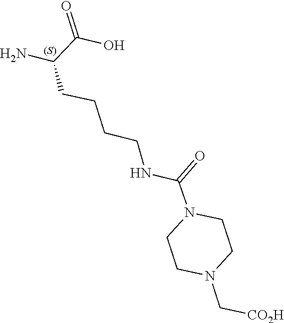 | 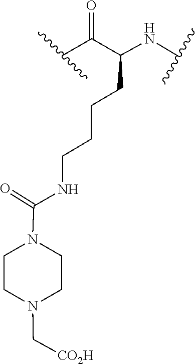 [SMILES: *N[C@@H](CCCCNC(=O)N1CCN(CC(=O)O)CC1)C(*)=O] |
| 3PyA(6NH2) | 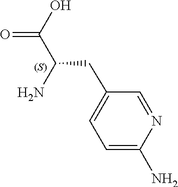 | 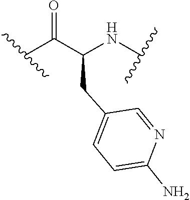 [SMILES: *N[C@@H](Cc1ccc(N)nc1)C(*)=O] |
| MeK(COpipzaa) | 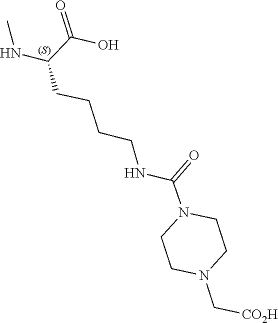 | 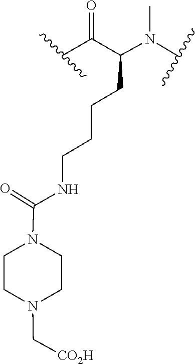 [SMILES: *C(=O)[C@H](CCCCNC(=O)N1CCN(CC(=O)O)CC1)N(*)C] |
| Dab(COPipzaa) | 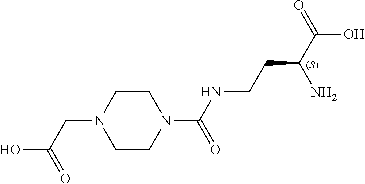 | 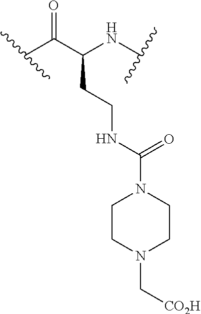 [SMILES: *N[C@@H](CCNC(=O)N1CCN(CC(=O)O)CC1)C(*)=O] |
| Orn(COPipzaa) | 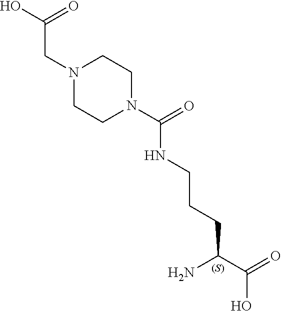 | 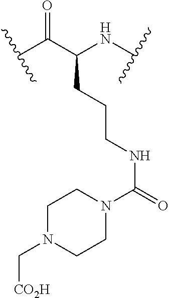 [SMILES: *N[C@@H](CCCNC(=O)N1CCN(CC(=O)O)CC1)C(*)=O] |
| Q(Pyrro(2CN4F2)) | 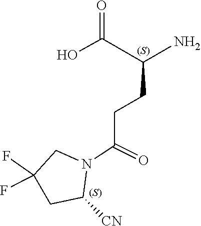 | 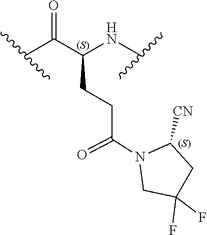 |
| Q(Pyrro(4F2)) | 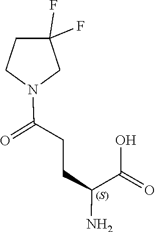 | 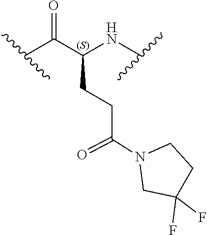 |
| hQ(Pyrro(4F2)) | 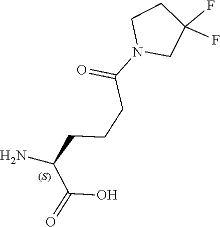 | |
| hQ(Pyrro(2CN4F2)) | 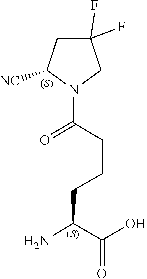 | |
| N(Pyrro(4F2)) | 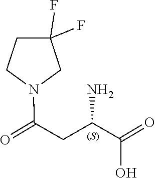 | |
| N(Pyrro(2CN4F2)) | 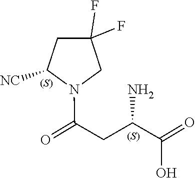 | |
| N(4Pyrro) | 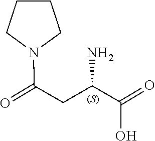 | |
| Glu(Alb1) | 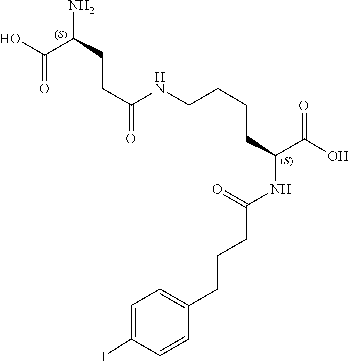 | |
| Glu(Alb2) | 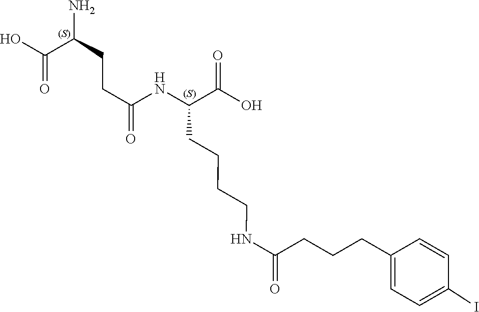 | |
| F(4CCH) | 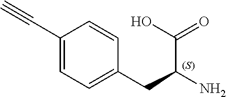 | |
| Trp(Boc) | 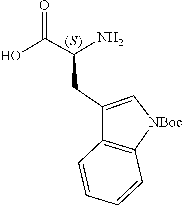 | 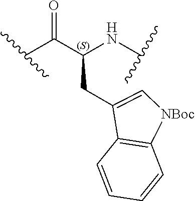 |
| K(COpipzaa)(OtBu) | 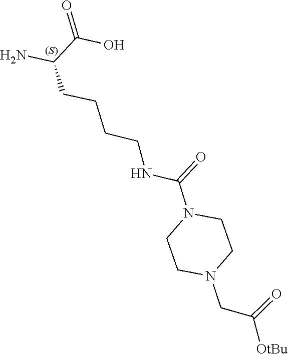 | 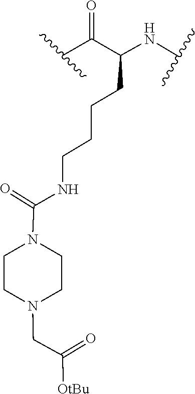 [SMILES: *N[C@@H](CCCCNC(=O)N1CCN(CC(=O)OC(C)(C)C)CC1)C(*)=O] |
| Asp(OMpe) | 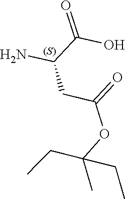 | 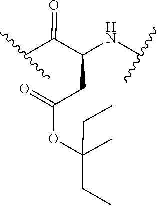 [SMILES: *N[C@@H](CC(=O)OC(C)(CC)CC)C(*)=O] |

| | |
|---|---|
| F(4amPEG2NHCO Py3(4F)) | 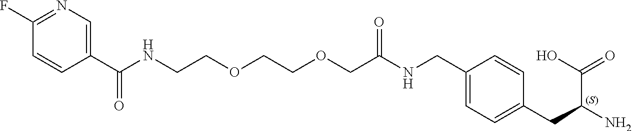 |
| F(4amPEG2NHCO Py3(4(NMe)3) | 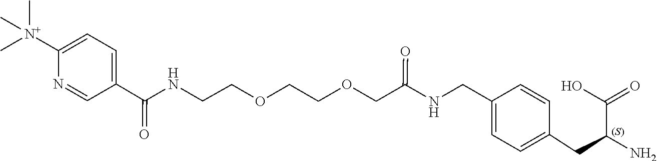 |
| F((4amPEG2NH)2- bE-DOTA) | 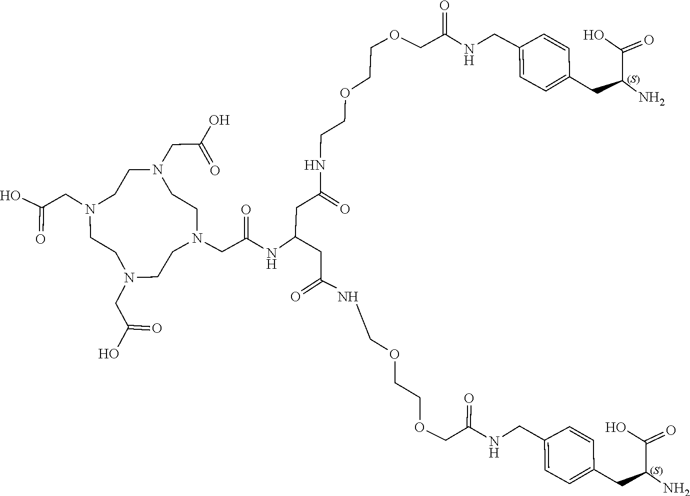 |
| F((4OEtNH- PEG3NH)2-bE- DOTA) | 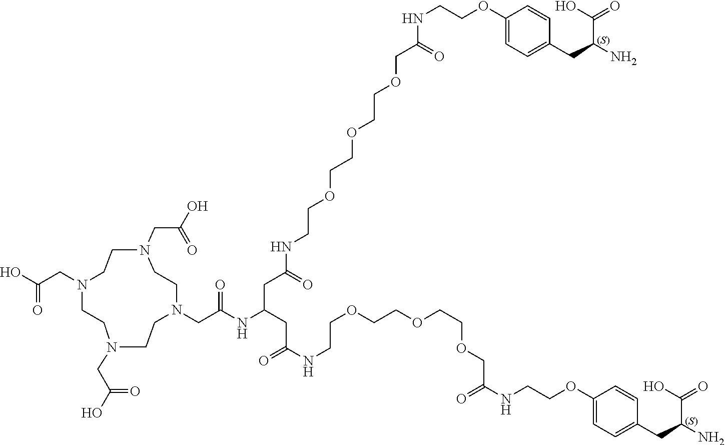 |

Amino acids of the D-isomeric form may be located at any of the positions in the FAP-targeting compounds disclosed herein (e.g., any of A¹-A¹⁰ appearing in the compounds of Formulae Peptides may be naturally occurring, synthetically produced, or recombinantly expressed.

Peptides may also comprise additional groups modifying the amino acid chain, for example, functional groups added via post-modification. Examples of post-modifications include, but are not limited to, acetylation, alkylation (including, methylation), biotinylation, glutamylation, glycylation, glycosylation, isoprenylation, lipoylation, phosphopantetheinylation, phosphorylation, selenation, and C-terminal amidation. The term peptide also includes peptides comprising modifications of the amino terminus and/or the carboxy terminus. The term peptide also includes modifications, such as, but not limited to, those described above, of amino acids falling between the amino and carboxy termini.

The skilled artisan will recognize that the peptide sequences disclosed herein in some cases are depicted having the left end of the sequence being the N-terminus of the peptide and the right end of the sequence being the C-terminus of the peptide, or in other cases are depicted having the left end of the sequence being the C-terminus of the peptide and the right end of the sequence being the N-terminus of the peptide. The context of the use of the sequence will make such directionality clear. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the disclosure, a C-terminal “—OH” moiety may be substituted for a C-terminal “—NH₂” moiety, and vice-versa.

The phrase “amino acid,” “amino acid residue,” or “residue” as used herein refers to an amino acid, a modified amino acid, an amino acid analog, or an amino acid mimetic that is incorporated into a peptide by an amide bond or an amide bond mimetic.

Unless indicated otherwise the names of naturally occurring and non-naturally occurring amino acid residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

One of skill in the art will appreciate that certain amino acids and other chemical moieties are modified when bound to another molecule. For example, an amino acid side chain may be modified when it forms an intramolecular bridge with another amino acid side chain, e.g., one or more hydrogens may be removed or replaced by the bond.

In some embodiments, amino acid residues in the disclosed cyclic peptides may exist in the zwitterionic form. As will be appreciated by one of skill in the art, a zwitterion is a molecule that contains both a positive charge and a negative charge, resulting in an overall neutral charge. For example, an amino acid in the zwitterion form includes a carboxylate ion (negative charge) and an ammonium ion (positive charge). Zwitterionic amino acids may exist at neutral pH.

As used herein, “about” means within ±10% of a value.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” means physiologically and pharmaceutically acceptable salts of the compounds disclosed herein, i.e., salts that retain the desired biological activity of the compounds and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of the compounds disclosed herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

As used herein, the term “solvate” means a physical association of a compound disclosed herein with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolable solvates. In general, such solvents selected for the purpose of the disclosure do not interfere with the biological activity of the solute. Non-limiting examples of suitable solvates include hydrates, ethanolates, methanolates, and the like.

As used herein, the term “hydrate” means a solvate wherein the solvent molecule(s) is/are water. In an embodiment, the solvate is a hydrate.

As used herein, the term “stereoisomer” means a molecule that has the same molecular formula and sequence of bonded atoms but differs in the three-dimensional orientations of its atoms in space.

The term “tautomer” means two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

As used herein, the term “treat,” “treatment,” or “treating” means decrease, suppress, attenuate, diminish, arrest, or stabilize the development or progression of a disorder or disease. As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “pharmaceutical composition” is defined herein to refer to a mixture (e.g., a solution or an emulsion) containing at least one active ingredient or therapeutic agent to be administered to a subject, e.g., a human, in order to prevent or treat a particular disease or condition affecting the subject.

The term “a therapeutically effective amount” of a compound (i.e., a compound of Formula (I), or a pharmaceutically acceptable salt thereof) of the present disclosure refers to an amount of the compound of the present disclosure that will elicit the biological or medical response of a subject (patient or subject), for example, reduction or inhibition of enzymatic, protein, or cellular activity, or ameliorate symptoms, alleviate conditions, slow or delay disease progression. The therapeutically effective dosage of a compound, the pharmaceutical composition, or the combinations thereof, is dependent on the ethnicity of the patient, the body weight, age, sex, and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The term “detectably effective amount” of a compound (i.e., a compound of Formula (I) or a pharmaceutically acceptable salt thereof) as used herein refers to an amount of the compound of the present disclosure sufficient to provide an acceptable image using equipment that is available in a clinical setting.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. A prefix such as “Cᵤ₋ᵥ” or (Cᵤ-Cᵥ) indicates that the following group has from u to v carbon atoms. For example, “C₁₋₆-alkyl” and “C₁-C₆ alkyl” both indicate that the alkyl group has from 1 to 6 carbon atoms.

The term “alkyl” is a straight or branched saturated hydrocarbon. For example, an alkyl group can have 1 to 10 carbon atoms (i.e., C₁₋₁₀-alkyl), 1 to 8 carbon atoms (i.e., C₁₋₈-alkyl), 1 to 5 carbon atoms (i.e., C₁₋₅-alkyl), 1 to 4 carbon atoms (i.e., C₁₋₄-alkyl), or 1 to 3 carbon atoms (i.e., C₁₋₃-alkyl). Examples of alkyl groups include, but are not limited to, methyl (Me, —CH₃), ethyl (Et, —CH₂CH₃), 1-propyl (n-Pr, n-propyl, —CH₂CH₂CH₃), isopropyl (i-Pr, i-propyl, —CH(CH₃)₂), 1-butyl (n-bu, n-butyl, —CH₂CH₂CH₂CH₃), 2-butyl (s-bu, s-butyl, —CH(CH₃)CH₂CH₃), tert-butyl (t-bu, t-butyl, —CH(CH₃)₃), 1-pentyl (n-pentyl, —CH₂CH₂CH₂CH₂CH₃), 2-pentyl (—CH(CH₃) CH₂CH₂CH₃), neopentyl (—CH₂C(CH₃)₃), 1-hexyl (—CH₂CH₂CH₂CH₂CH₂CH₃), 2-hexyl (—CH(CH₃)CH₂CH₂CH₂CH₃), heptyl (—(CH₂)₆CH₃), octyl (—(CH₂)₇CH₃), 2,2,4-trimethylpentyl (—CH₂C(CH₃)₂CH₂CH(CH₃)₂), nonyl (—(CH₂)₈CH₃), decyl (—(CH₂)₉CH₃), undecyl (—(CH₂)₁₀CH₃), and dodecyl (—(CH₂)₁₁CH₃). In an embodiment, alkyl refers to C₁₋₆-alkyl. In another embodiment, alkyl refers to C₁₋₄alkyl. In another embodiment, alkyl refers to C₁₋₃alkyl.

The term “alkylene” refers to a bivalent alkyl group. For example, an alkylene group can have 1 to 10 carbon atoms (i.e., (C₁₋₁₀alkylene), 1 to 5 carbon atoms (i.e., (C₁₋₅alkylene), 1 to 2 carbon atoms (i.e., (C1-2alkylene), or 1 carbon atom (i.e., C₁-alkylene). Examples of alkylene groups include, but are not limited to, methylene (—CH₂—), ethylene (—CH₂CH₂—), n-propylene (—CH₂CH₂CH₂—), n-butylene (—CH₂CH₂CH₂CH₂—), etc.

The term “acyl” refers to a substituent containing a carbonyl moiety and a non-carbonyl moiety and is meant to include an amino-acyl. The carbonyl moiety contains a double-bond between the carbonyl carbon and an oxygen heteroatom. The non-carbonyl moiety is selected from straight, branched, and cyclic alkyl, which includes, but is not limited to, a straight, branched, or cyclic C₁₋₂₀ alkyl, C₁₋₁₀ alkyl, or C₁₋₆ alkyl. In a non-limiting example, acyl is “2-7 acyl,” which refers to an acyl group in which the non-carbonyl moiety comprises C₁₋₆ alkyl. Examples of C₂₋₇-acyl, include, but are not limited to C(O)CH₃, C(O)CH₂CH₃, C(O)CH(CH₃)₂, C(O)CH(CH₃)CH₂CH₃, and C(O)C(CH₃)₃.

The term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, biphenyl, and naphthyl. In some embodiments, aryl groups have from six to sixteen carbon atoms (e.g., C₆₋₁₆-aryl). In some embodiments, aryl groups have from six to twelve carbon atoms (e.g., C₆₋₁₂-aryl). In some embodiments, aryl groups have six carbon atoms (e.g., C₆-aryl, which may also be referred to as phenyl).

The term “halo” or “halogen” refers to bromo (—Br), chloro (—Cl), fluoro (—F), or iodo (—I). In an embodiment, halo refers to fluoro.

The term “haloalkyl” refers to a straight- or branched-chain alkyl group having from 1 to 12 carbon atoms, 1 to 6 carbon atoms, 1 to 4 carbon atoms, or 1 to 3 carbon atoms in the chain optionally substituting one or more H with halo. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that, in light of the ordinary skill in the art and the teachings provided herein, would be considered equivalent to any one of the foregoing examples. In an embodiment, haloalkyl refers to C₍₁₋₆₎haloalkyl. In another embodiment, haloalkyl refers to C₍₁₋₄₎haloalkyl. In another embodiment, alkyl refers to C₍₁₋₃₎haloalkyl.

The term “cycloalkyl” refers to a saturated or partially unsaturated all carbon ring system having 3 to 8 carbon atoms (i.e., C₃₋₈cycloalkyl), or 3 to 6 carbon atoms (i.e., C₃₋₆cycloalkyl), wherein the cycloalkyl ring system has a single ring or multiple rings, e.g., in a spirocyclic or bicyclic form. Exemplary cycloalkyls include, but are not limited to, cyclopropyl, cyclobutyl, cyclopentyl, cyclohexyl, cycloheptyl, and cyclooctyl. Some cycloalkyl groups may exist as spirocycloalkyls, wherein two cycloalkyl rings are fused through a single carbon atom; for example and without limitation, an example of a spiropentyl group is

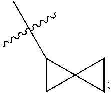 [SMILES: *C1CC12CC2]

for example and without limitation, examples of spirohexyl groups include

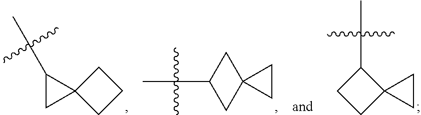 [SMILES: *C1CC12CCC2 ; *C1CC2(CC2)C1 ; *C1CCC12CC2]

for example and without limitation examples of cycloheptyl groups include

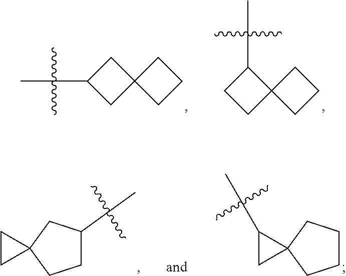

for example and without limitation examples of cyclooctyl groups include

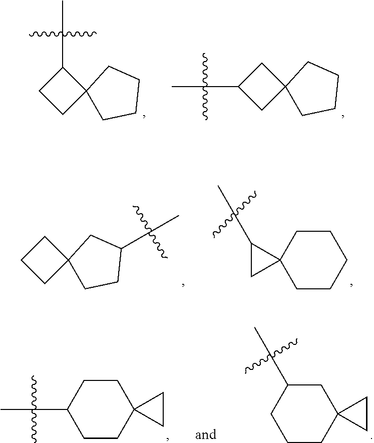 [SMILES: *C1CCC12CCCC2 ; *C1CC2(CCCC2)C1 ; *C1CCC2(CCC2)C1 ; *C1CC12CCCCC2 ; *C1CCC2(CC1)CC2 ; *C1CCCC2(CC2)C1]

Bicyclic cycloalkyl ring systems also include

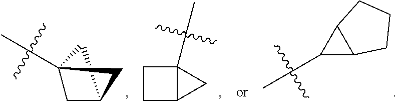

The term “heterocycle” or “heterocyclyl” refers to a saturated or partially unsaturated ring system that has at least one atom other than carbon in the ring system, wherein the atom is selected from the group consisting of oxygen, nitrogen and sulfur. The heterocyclyl group may, for example, consist of a single ring or multiple rings (e.g., in the form of a spirocyclic or bicyclic ring system). Exemplary heterocycles include, but are not limited to oxetanyl, aziridinyl, azetidinyl, pyrrolidinyl, piperidinyl, piperazinyl, morpholinyl, tetrahydropyranyl, tetrahydrofuranyl, and thiomorpholinyl.

As used herein, the term “heteroaryl” or “heteroaromatic,” employed alone or in combination with other terms, refers to a heterocycle having aromatic character. Heteroaryl substituents may be defined by the number of carbon atoms, e.g., C₁₋₉-heteroaryl indicates the number of carbon atoms contained in the heteroaryl group without including the number of heteroatoms. For example, a C₁₋₉-heteroaryl will include an additional one to four heteroatoms. Alternatively, heteroaryl substituents may be defined by the number of atoms in the heteroaryl core, e.g., 5-6 membered heteroaryl indicates the number of carbon and heteroatoms contained in the heteroaryl core. As used herein, heteroaryl includes polycyclic ring systems wherein at least one ring has aromatic character and thus, may include one or more rings that are partially saturated. Non-limiting examples of heteroaryls include pyridyl, pyrazinyl, pyrimidinyl (including, e.g., 2- and 4-pyrimidinyl), pyridazinyl, thienyl, furyl, pyrrolyl (including, e.g., 2-pyrrolyl), imidazolyl, thiazolyl, oxazolyl, pyrazolyl (including, e.g., 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

When two terms are combined, such as, for example, “alkylaryl” and “alkylheteroaryl,” it is intended that the elements defined by those terms are covalently attached, and that such covalent attachment may occur at any site of each element. For example, C₁₋₆-alkyl(5 to 6 membered heteroaryl) indicates a C₁₋₆-alkyl that is covalently attached to a 5 to 6 membered heteroaryl. In some embodiments, the 5 to 6 membered heteroaryl may be covalently attached to the C₁₋₆-alkyl at the C1 or C6 carbon, thereby providing a linear alkylheteroaryl group. In some embodiments, the 5 to 6 membered heteroatom is covalently attached to the C₁₋₆-alkyl at any one of C2, C3, C4, and C5 carbon atoms, thereby providing a branched alkylheteroaryl group. Likewise, the C₁₋₆-alkyl may be attached to any atom of the 5 to 6 membered heteroaryl. Further, a substituent defined using combined terms, e.g., “alkylaryl” and “alkylheteroaryl,” may be connected to the compound via any atom of either of the elements defined by the combined terms. For example, an “alkylaryl” or “alkylheteroaryl” substituent may be connected to the compound via the alkyl group or the aryl/heteroaryl group. In various embodiments, an “alkylaryl” or “alkylheteroaryl” substituent is connected to the compound via the alkyl group. In various other embodiments, the “alkylaryl” or “alkylheteroaryl” substituent is connected to the compound via the aryl or heteroaryl group.

The terms “connected to” and “conjugated to” as used herein may be used interchangeably and are meant to indicate that two independent constituents are joined together such as by one or more covalent bonds. In some embodiments, the cyclized peptide is conjugated to or connected to a chelating agent via a covalent bond.

The terms “chelated to” and “complexed to” as used herein may be used interchangeably and are meant to indicate that two independent constituents are joined together such as by one or more non-covalent bonds, e.g., coordination bonds.

The term “radiolabeled” as used herein means that a non-radioactive compound is labeled with a radioisotope. Radiolabeling can be achieved, e.g., via chelation or complexation of a chelator with an appropriate radionuclide. Radiolabeling can also refer to chemically substituting one group on a compound for a radionuclide, such as, e.g., in the case of ¹⁸F.

Furthermore, it is intended that within the scope of the present invention, any element, in particular when mentioned in relation to a peptide of the disclosure, or pharmaceutically acceptable salt thereof, shall comprise all isotopes and isotopic mixtures of said element, either naturally occurring or synthetically produced, either with natural abundance or in an isotopically enriched form. For example, a reference to hydrogen includes within its scope ¹H, ²H (i.e., deuterium or D), and ³H (i.e., tritium or T). In some embodiments, the compounds described herein include a ²H (i.e., deuterium) isotope. By way of example, the group denoted —C₍₁₋₆₎alkyl includes not only —CH₃, but also CD₃; not only CH₂CH₃, but also CD₂CD₃, etc. Similarly, references to carbon and oxygen include within their scope respectively ¹²C, ¹³C and ¹⁴C and ¹⁵O and ¹⁶O and ¹⁷O and ¹⁸O. The isotopes may be radioactive or non-radioactive.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, stabilizers (i.e., a pharmaceutically acceptable stabilizer), binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the imaging, therapeutic, or pharmaceutical compositions is contemplated.

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

### FAP-Targeting Compounds

The present disclosure provides FAP-targeting compounds comprising:
a) at least one cyclized peptide

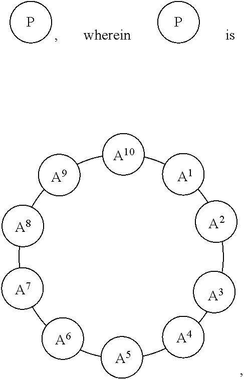

wherein A¹-A¹⁰ are as defined herein; and
b) at least one imaging agent, chelating agent, radionuclide, or cytotoxic drug, wherein the cyclized peptide

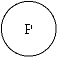

conjugated to the at least one imaging agent, chelating agent, radionuclide, or cytotoxic drug via any one of A¹-A¹⁰, optionally through a linker.

In some embodiments, the FAP-targeting compound is a compound formula (I), (Ia), (Ib), or (Ic):

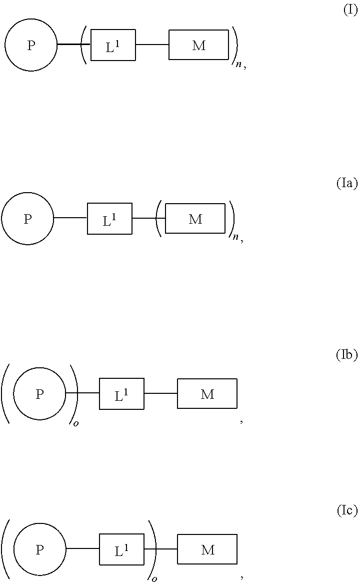

or a pharmaceutically acceptable salt, solvate, stereoisomer, or tautomer thereof, wherein:
P is a cyclized peptide;
L¹ is, independently at each occurrence, a bond or a linker;
M is, independently at each occurrence, an imaging agent, a chelating agent, or a radionuclide, or cytotoxic drug,
wherein the chelating agent is optionally radiolabeled with a radionuclide;
n is 1, 2, 3, or 4; and
is 1, 2, 3, or 4,
where any of P, L¹, and M is optionally substituted with an albumin binder.

In some embodiments, provided herein is a compound of formula (Id)

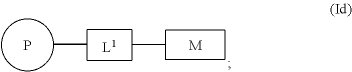

or a pharmaceutically acceptable salt, solvate, stereoisomer, or tautomer thereof,
wherein:
P is a cyclized peptide;
L¹ is, independently at each occurrence, a bond or a linker; and
M is, independently at each occurrence, an imaging agent, a chelating agent, or a radionuclide, or cytotoxic drug,
wherein the chelating agent is optionally radiolabeled with a radionuclide, and
where any of P, L¹, and M is optionally substituted with an albumin binder.

In some embodiments, L¹ is a bond. In some embodiments, L¹ is a linker.

In some embodiments of the compounds of compound formula (I), (Ia), (Ib), (Ic), or (Id), or a pharmaceutically acceptable salt, solvate, stereoisomer, or tautomer thereof, M is, independently at each occurrence, an imaging agent, a chelating agent, or a radionuclide, wherein the chelating agent is optionally radiolabeled with a radionuclide. In further embodiments, at least one M is a chelating agent, wherein the chelating agent is radiolabeled with a radionuclide. In further embodiments, at least one M is an imaging agent. In some embodiments, at least one M is a radionuclide.

In some embodiments of the compounds of compound formula (I), (Ia), (Ib), (Ic), or (Id), or a pharmaceutically acceptable salt, solvate, stereoisomer, or tautomer thereof, n is 1, 2, 3, or 4. In further embodiments, n is 1, 2, or 3. In further embodiments, n is 1 or 2. In further embodiments, n is 1. In further embodiments, n is 2. In further embodiments, n is 3. In further embodiments, n is 4.

In some embodiments of the compound of formula (I), (Ia), (Ib), (Ic), or (Id), or a pharmaceutically acceptable salt, solvate, stereoisomer, or tautomer thereof, o is 1, 2, 3, or 4. In further embodiments, o is 1, 2, or 3. In further embodiments, o is 1 or 2. In further embodiments, o is 1. In further embodiments, o is 2. In further embodiments, o is 3. In further embodiments, o is 4.

In some embodiments of the compound of formula (I), (Ia), (Ib), (Ic), or (Id), or a pharmaceutically acceptable salt, solvate, stereoisomer, or tautomer thereof, the compound is substituted with an albumin binder. In further embodiments, P is substituted with an albumin binder. In further embodiments, L¹ is substituted with an albumin binder. In further embodiments, M is substituted with an albumin binder. In some embodiments, none of P, L¹, and M are substituted with an albumin binder (i.e., the compound of formula (I), (Ia), (Ib), (Ic), or (Id), or a pharmaceutically acceptable salt, solvate, stereoisomer, or tautomer thereof is not substituted with an albumin binder).

In some embodiments, the FAP-targeting compound is a compound of formula (I):

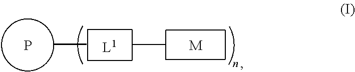

or a pharmaceutically acceptable salt, solvate, stereoisomer, or tautomer thereof.

In some embodiments, the FAP-targeting compound is a compound of formula (Ib):

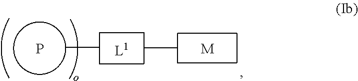

or a pharmaceutically acceptable salt, solvate, stereoisomer, or tautomer thereof.
Cyclic Peptide (P) and Incorporation into, e.g., Compounds of Formulae (I), (Ia), (Ib), (Ic), and (Id)

In some aspects, the present disclosure provides a cyclic peptide (P) that is capable of binding to fibroblast activation protein (FAP), as well as compounds comprising said cyclic peptide (P). In one or more embodiments, P has the structure:

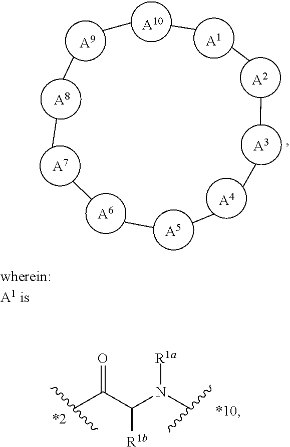

wherein
R^{1a} is selected from the group consisting of H and C₁₋₃-alkyl;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1b} is optionally substituted with 1, 2, 3, or 4 substituents independently selected from the group consisting of C₁₋₃-alkyl, —NH₂, —NHC₁₋₆-alkyl, —N(C₁₋₆-alkyl)₂, —OH, halo, and C(O)R^{1d};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH;
R^{1d} is selected from the group consisting of H, —OH, C₁₋₆-alkyl —NHC₁₋₆-alkyl, and —OC₁₋₆-alkyl; and
*2 indicates the point of attachment to A² and *10 indicates the point of attachment to A¹⁰;
A² is an amino acid or derivative thereof;
A³ is

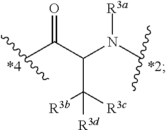

wherein
R^{3a} is selected from the group consisting of H and C₁₋₃-alkyl;
R^{3b} is selected from the group consisting of H, C₁₋₃-alkyl, and halo;
R^{3c} is selected from the group consisting of halo, C₁₋₆ alkyl, C₃₋₈-cycloalkyl, phenyl, and 5-6 membered heteroaryl, wherein the C₁₋₆ alkyl, phenyl, and 5-6 membered heteroaryl of R^{3c} are optionally substituted with 1, 2, 3, or 4 substituents independently selected from the group consisting of halo, —C(O)OH, —C(O)C₁₋₆-alkyl, —OH, —NHC₁₋₆-alkyl, —N(C₁₋₆-alkyl)₂, and —NH₂;
R^{3d} is selected from the group consisting of H, C₁₋₃-alkyl, and halo; and
*2 indicates the point of attachment to A² and *4 indicates the point of attachment to A⁴;
A⁴ is

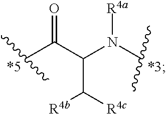

wherein
R^{4a} is selected from the group consisting of H and C₁₋₃-alkyl;
R^{4b} is selected from the group consisting of H and C₁₋₃-alkyl;
R^{4c} is selected from the group consisting of phenyl and 5-6 membered heteroaryl wherein phenyl and 5-6 membered heteroaryl of R⁴ are optionally substituted with 1, 2, 3, 4, or 5 substituents R^{4d};
each R^{4d} is independently selected from C₁₋₆-alkyl, —OH, —OC₁-C₆-alkyl, —NH₂, halo, —NHC(O)R^{4f}, —C(O)NHR^{4f}, and —C(O)OR^{4f}, wherein the C₁₋₆-alkyl and —OC₁-C₆-alkyl of R^{4d} are each optionally substituted with 1, 2, or 3 substituents R^{4e};
each R^{4e} is independently selected from —CN, —NH₂, —NHC₁₋₆-alkyl, —N(C₁₋₆-alkyl)₂, —NHC(O)—(C₁₋₆-alkyl), —N₃, —OH, —C(O)OH, —C(O)NH₂, and 5-6 membered heteroaryl, wherein the 5-6 membered heteroaryl of R⁴ is optionally substituted with —C₁₋₆-alkyl-NH₂ or —C₁₋₃-alkyl-OH;
each R^{4f} is independently selected from the group consisting of H and C₁₋₆-alkyl; and
*3 indicates the point of attachment to A³ and *5 indicates the point of attachment to A⁵;
A⁵ is

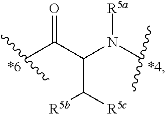

wherein
R^{5a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of R, is optionally substituted with halo, —OH, —OC₁₋₆-alkyl, and —C(O)R^{5d};
R^{5d} is 4-7 membered heterocycloalkyl optionally substituted with 1, 2, 3, or 4 groups independently selected from C₁₋₃-alkyl, —CN, halo, and —OH; and
wherein *4 indicates the point of attachment to A⁴ and *6 indicates the point of attachment to A⁶;
A⁶ is

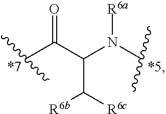

wherein
R^{6a} is selected from the group consisting of H and C₁₋₃-alkyl;
R^{6b} is selected from the group consisting of H and C₁₋₃-alkyl;
R^{6c} is a 5 to 10 membered heteroaryl optionally substituted with 1, 2, 3, or 4 groups independently selected from C₁₋₃-alkyl, —CN, halo, —OH, and —OC₁₋₆-alkyl; and
wherein *5 indicates the point of attachment to A⁵ and *7 indicates the point of attachment to A⁷;
A⁷ is an amino acid or derivative thereof;
A⁸ is

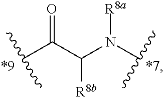

wherein
R^{8a} is selected from the group consisting of H, C₁₋₃-alkyl, and —CH₂-phenyl, wherein the C₁₋₃-alkyl and —CH₂-phenyl of R^{8a} are each optionally substituted with 1, 2, or 3 substituents independently selected from —NH₂, —NHC₁₋₆-alkyl, —N(C₁₋₆-alkyl)₂, —OH, —OC₁₋₃-alkyl, and C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆-alkyl-O—C(O)R^{8c}, and —C₁₋₆-alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH; and
wherein *7 indicates the point of attachment to A⁷ and *9 indicates the point of attachment to A⁹;
A⁹ is an amino acid or derivative thereof; and
A¹⁰ is

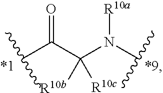

wherein
R^{10a} is selected from the group consisting of H and C₁₋₃-alkyl;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1, 2, 3, or 4 substituents independently selected from C₁₋₃-alkyl, —NH₂—NHC₁₋₆-alkyl, —N(C₁₋₆-alkyl)₂, —OH, halo, and C(O)R^{10d};
R^{10d} is selected from the group consisting of H, —OH, C₁₋₆-alkyl and —OC₁₋₆-alkyl; and
wherein *1 indicates the point of attachment to A¹ and *9 indicates the point of attachment to A⁹.

In some embodiments, the compound of the present disclosure is or comprises a cyclic peptide (P):

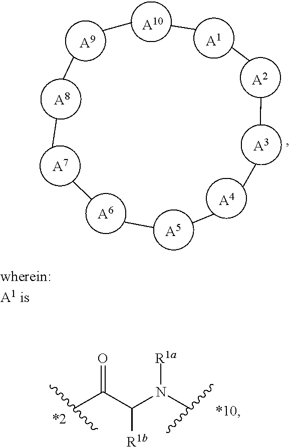

wherein
R^{1a} is selected from the group consisting of H and C₁₋₃-alkyl;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH₂, —NHC₁₋₆-alkyl, —N(C₁₋₆-alkyl)₂ and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1b} is optionally substituted with 1, 2, 3, or 4 substituents independently selected from the group consisting of C₁₋₃-alkyl, —NH₂, —NHC₁₋₆-alkyl, —N(C₁₋₆-alkyl)₂, —OH, halo, and C(O)R^{1d};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH;
R^{1d} is selected from the group consisting of H, —OH, C₁₋₆-alkyl, —NHC₁₋₆-alkyl, and —OC₁₋₆-alkyl; and
*2 indicates the point of attachment to A² and *10 indicates the point of attachment to A¹⁰;
A² is

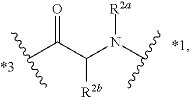

wherein
R^{2a} is selected from the group consisting of H and C₁₋₃-alkyl;
R^{2b} is selected from the group consisting of H and C₁₋₆-alkyl, wherein the C₁₋₆-alkyl of R^{2b} is optionally substituted with 1, 2, 3, or 4 substituents independently selected from the group consisting of —C(O)OH, —OH, —NH₂, —NHC₁₋₆-alkyl, and —N(C₁₋₆-alkyl)₂; and
*1 indicates the point of attachment to A¹ and *3 indicates the point of attachment to A³;
A³ is

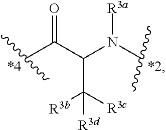

wherein
R^{3a} is selected from the group consisting of H and C₁₋₃-alkyl;
R^{3b} is selected from the group consisting of H, C₁₋₃-alkyl, and halo;
R^{3c} is selected from the group consisting of halo, C₁₋₆ alkyl, C₃₋₈-cycloalkyl, phenyl, and 5-6 membered heteroaryl, wherein the C₁₋₆ alkyl, phenyl, and 5-6 membered heteroaryl of R^{3c} are optionally substituted with 1, 2, 3, or 4 substituents independently selected from the group consisting of halo, —C(O)OH, —C(O)C₁₋₆-alkyl, —OH, —NHC₁₋₆-alkyl, —N(C₁₋₆-alkyl)₂, and —NH₂;
R^{3d} is selected from the group consisting of H, C₁₋₃-alkyl, and halo; and
*2 indicates the point of attachment to A² and *4 indicates the point of attachment to A⁴;
A⁴ is

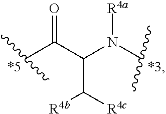

wherein
R^{4a} is selected from the group consisting of H and C₁₋₃-alkyl;
R^{4b} is selected from the group consisting of H and C₁₋₃-alkyl;
R^{4c} is selected from the group consisting of phenyl and 5-6 membered heteroaryl wherein phenyl and 5-6 membered heteroaryl of R⁴ are optionally substituted with 1, 2, 3, 4, or 5 substituents R^{4d};
each R^{4d} is independently selected from C₁₋₆-alkyl, —OH, —OC₁-C₆-alkyl, —NH₂, halo, —NHC(O)R^{4f}, —C(O)NHR^{4f}, and —C(O)OR^{4f}, wherein the C₁₋₆-alkyl and —OC₁-C₆-alkyl of R^{4d} are each optionally substituted with 1, 2, or 3 substituents R^{4e};
each R^{4c} is independently selected from —CN, —NH₂, —NHC₁₋₆-alkyl, —N(C₁₋₆-alkyl)₂, —NHC(O)—(C₁₋₆-alkyl), —N₃, —OH, —C(O)OH, —C(O)NH₂, and 5-6 membered heteroaryl, wherein the 5-6 membered heteroaryl of R⁴ is optionally substituted with —C₁₋₆-alkyl-NH₂ or —C₁₋₃-alkyl-OH;
each R^{4f} is independently selected from the group consisting of H and C₁₋₆-alkyl; and
*3 indicates the point of attachment to A³ and *5 indicates the point of attachment to A⁵;
A⁵ is

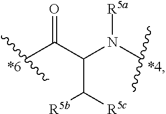

wherein
R^{5a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, and —C(O)R^{5d};
R^{5d} is 4-7 membered heterocycloalkyl optionally substituted with 1, 2, 3, or 4 groups independently selected from C₁₋₃-alkyl, —CN, halo, and —OH; and
wherein *4 indicates the point of attachment to A⁴ and *6 indicates the point of attachment to A⁶;
A⁶ is

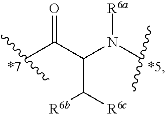

wherein
R^{6a} is selected from the group consisting of H and C₁₋₃-alkyl;
R^{6b} is selected from the group consisting of H and C₁₋₃-alkyl;
R^{6c} is a 5-10 membered heteroaryl optionally substituted with 1, 2, 3, or 4 groups independently selected from C₁₋₃-alkyl, —CN, halo, —NH₂, —NHC₁₋₆-alkyl, —N(C₁₋₆-alkyl)₂, —OH, and —OC₁₋₆-alkyl; and
wherein *5 indicates the point of attachment to A⁵ and *7 indicates the point of attachment to A⁷;
A⁷ is

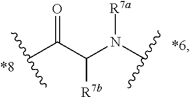

wherein
R^{7a} is selected from the group consisting of H and C₁₋₃-alkyl;
R^{7b} is selected from the group consisting of H and C₁₋₆-alkyl; and
wherein *6 indicates the point of attachment to A⁶ and *8 indicates the point of attachment to A⁸;
A⁸ is

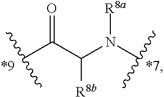

wherein
R^{8a} is selected from the group consisting of H, C₁₋₃-alkyl, and —CH₂-phenyl, wherein the C₁₋₃-alkyl and —CH₂-phenyl of R^{8a} are each optionally substituted with 1, 2, or 3 substituents independently selected from —NH₂, —NHC₁₋₆-alkyl, —N(C₁₋₆-alkyl)₂, —OH, —OC₁₋₃-alkyl, and C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆alkyl-O—C(O)R^{8c}, and —C₁₋₆alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH and
wherein *7 indicates the point of attachment to A⁷ and *9 indicates the point of attachment to A⁹;
A⁹ is

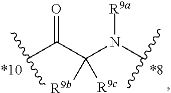

wherein
R^{9a} is selected from the group consisting of H and C₁₋₃-alkyl;
R^{9b} is selected from the group consisting of H and C₁₋₃-alkyl;
R^{9c} is C₁₋₆-alkyl optionally substituted with 1, 2, 3, or 4 groups independently selected from —CN, —C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
wherein *8 indicates the point of attachment to A⁸ and *10 indicates the point of attachment to A¹⁰;
A¹⁰ is

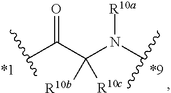

wherein
R^{10a} is selected from the group consisting of H and C₁₋₃-alkyl;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1, 2, 3, or 4 substituents independently selected from C₁₋₃-alkyl, —NH₂, —NHC₁₋₆-alkyl, —N(C₁₋₆-alkyl)₂, —OH, halo, and C(O)R^{10d};
R^{10d} is selected from the group consisting of H, —OH, C₁₋₆-alkyl and —OC₁₋₆-alkyl; and
wherein *1 indicates the point of attachment to A¹ and *9 indicates the point of attachment to A⁹.

In some embodiments of the cyclic peptide (P), or compounds comprising cyclic peptide (P):
A¹ is

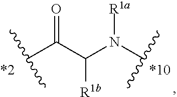

wherein
R^{1a} is H or CH₃;
R^{1b} is selected from the group consisting of H and C₁₋₆-alkyl, wherein the C₁₋₆-alkyl of R^{1b} is optionally substituted with a substituent selected from —C(O)OH, —OH, —NH₂, —NHC₁₋₆-alkyl, —N(C₁₋₆-alkyl)₂ and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1c} is optionally substituted with a substituent selected from C₁₋₃-alkyl, —NH₂, —NHC₁₋₆-alkyl, —N(C₁₋₆-alkyl)₂, and —OH; and
R^{1c} is selected from the group consisting of C₁₋₆-alkyl and 4-7 membered heterocycloalkyl, wherein the 4-7 membered heterocycloalkyl of R^{1c} is optionally substituted with a substituent selected from the group consisting of —C₁₋₆-alkyl-NH₂ and —C₁₋₆-alkyl-C(O)OH;
A² is

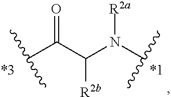

wherein
R^{2a} is H or CH₃; and
R^{2b} is selected from the group consisting of H and C₁₋₆-alkyl, wherein the C₁₋₆-alkyl of R^{2b} is optionally substituted with a substituent selected from —C(O)OH, —OH, —NH₂, —NHC₁₋₆-alkyl, and —N(C₁₋₆-alkyl)₂;
A³ is

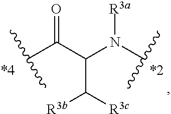

wherein
R^{3a} is H and CH₃;
R^{3b} is selected from the group consisting of H and C₁₋₃-alkyl; and
R^{3c} is selected from the group consisting of C₁₋₆ alkyl, C₃₋₈-cycloalkyl, phenyl, and 5-6 membered heteroaryl, wherein the phenyl and 5-6 membered heteroaryl of R^{3c} are optionally substituted with halo, —C(O)OH, —C(O)C₁₋₆-alkyl, —OH, —NHC₁₋₆-alkyl, —N(C₁₋₆-alkyl)₂, and —NH₂;
A⁴ is

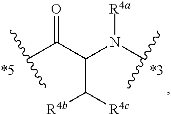

wherein
R^{4a} is H or CH₃;
R^{4b} is selected from the group consisting of H and C₁₋₃-alkyl,
R^{4c} is selected from the group consisting of phenyl and 5-6 membered heteroaryl wherein phenyl and 5-6 membered heteroaryl of R⁴ are substituted with a substituent R^{4d},
R^{4d} is selected from C₁₋₆-alkyl, —OH, and —OC₁-C₆-alkyl, wherein the C₁₋₆-alkyl and —OC₁-C₆-alkyl of R^{4d} are each optionally substituted with a substituent R^{4c}, and
R^{4c} is selected from —NH₂, —NHC₁₋₆-alkyl, —N(C₁₋₆-alkyl)₂, —NHC(O)—(C₁₋₆-alkyl), —N₃, and 5-6 membered heteroaryl, wherein the 5-6 membered heteroaryl of R⁴ is optionally substituted with —C₁₋₆-alkyl-NH₂ or —C₁₋₃-alkyl-OH; A⁵ is

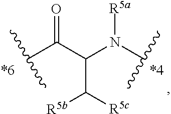

wherein
R^{5a} is H or CH₃;
R^{5b} is selected from the group consisting of H, C₁₋₃-alkyl, and —OH,
R^{5c} is selected from the group consisting of H and C₁₋₈-alkyl, wherein the C₁₋₈-alkyl of R^{5c} is optionally substituted with halo, —OH, —OC₁₋₆-alkyl, and —C(O)R^{5d}, and
R^{5d} is 4-7 membered heterocycloalkyl optionally substituted with 1, 2, or 3 groups independently selected from C₁₋₃-alkyl, —CN, halo, and —OH;
A⁶ is

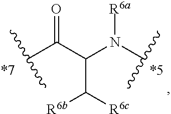

wherein
R^{6a} is H or CH₃;
R^{6b} is selected from the group consisting of H and C₁₋₃-alkyl, and
R^{6c} is a 5 to 10 membered heteroaryl optionally substituted with a group selected from C₁₋₃-alkyl, —CN, halo, —OH, and —OC₁₋₃-alkyl;
A⁷ is

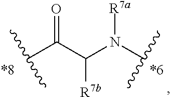

wherein
R^{7a} is H or CH₃; and
R^{7b} is selected from the group consisting of H and C₁₋₃-alkyl;
A⁸ is

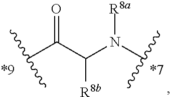

wherein
R^{8a} is selected from the group consisting of H, CH₃, and —CH₂-phenyl, wherein the —CH₂-phenyl of R^{8a} is optionally substituted with —NH₂, —NHC₁₋₆-alkyl, —N(C₁₋₆-alkyl)₂, —OH, —OC₁₋₃-alkyl, or C₁₋₃-alkyl;
R^{8b} is selected from the group consisting of H, C₁₋₆-alkyl, —CH₂-phenyl, —CH₂-(5 to 6 membered heteroaryl), and —C₁₋₆-alkyl-NH—C(O)R^{8c}, wherein C₁₋₆-alkyl, —CH₂-phenyl, and —CH₂-(5 to 6 membered heteroaryl) of R^{b} are optionally substituted with halo, —NHC₁₋₆-alkyl, —N(C₁₋₆-alkyl)₂, —NH₂, —OH, —OC₁₋₃-alkyl, or C₁₋₃-alkyl; and
R^{8c} is a 4-7 membered heterocycloalkyl optionally substituted with —C₁₋₆-alkylC(O)OH;
A⁹ is

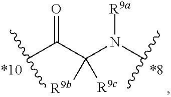

wherein
R^{9a} is H or CH₃;
R^{9b} is selected from the group consisting of H and C₁₋₃-alkyl, and
R^{9c} is C₁₋₆-alkyl optionally substituted with a group selected from —CN, —C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
A¹⁰ is

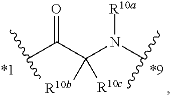

wherein
R^{10a} is H or CH₃;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents selected from —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1 or 2 substituents selected from C₁₋₃-alkyl, —NH₂, —NHC₁₋₆-alkyl, —N(C₁₋₆-alkyl)₂, and —OH; and
R^{10d} is selected from the group consisting of H, —OH, C₁₋₆-alkyl and —OC₁₋₆-alkyl.

In further embodiments, A¹ is

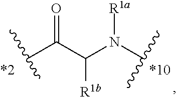

wherein
R^{1a} is selected from the group consisting of H and C₁₋₃-alkyl;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH₂, —NHC₁₋₆-alkyl, —N(C₁₋₆-alkyl)₂ and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1b} is optionally substituted with 1, 2, 3, or 4 substituents independently selected from the group consisting of C₁₋₃-alkyl, —NH₂, —NHC₁₋₆-alkyl, —N(C₁₋₆-alkyl)₂, —OH, halo, and C(O)R^{1d};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH; and
R^{1d} is selected from the group consisting of H, —OH, C₁₋₆-alkyl, —NHC₁₋₆-alkyl, and —OC₁₋₆-alkyl.

In further embodiments, A¹ is

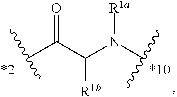

wherein
R^{1a} is H or CH₃;
R^{1b} is selected from the group consisting of H and C₁₋₆-alkyl, wherein the C₁₋₆-alkyl of R^{1b} is optionally substituted with a substituent selected from —C(O)OH, —OH, —NH₂, —NHC₁₋₆-alkyl, —N(C₁₋₆-alkyl)₂, and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1c} is optionally substituted with a substituent selected from C₁₋₃-alkyl —NH₂, —NHC₁₋₆-alkyl, —N(C₁₋₆-alkyl)₂, and —OH; and
R^{1c} is selected from the group consisting of C₁₋₆-alkyl and 4-7 membered heterocycloalkyl, wherein the 4-7 membered heterocycloalkyl of R^{1c} is optionally substituted with a substituent selected from the group consisting of —C₁₋₆-alkyl-NH₂ and —C₁₋₆-alkyl-C(O)OH.

In further embodiments, A¹ is

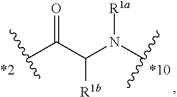

wherein
R^{1a} is H or CH₃;
R^{1b} is selected from the group consisting of H and C₁₋₆-alkyl, wherein the C₁₋₆-alkyl of R^{1b} is optionally substituted with a substituent selected from —C(O)OH, —OH, —NH₂, —NHC₁₋₆-alkyl, —N(C_{I}-6-alkyl)₂, and —NH—C(O)R^{1c};
R^{1c} is selected from the group consisting of C₁₋₆-alkyl and 4-7 membered heterocycloalkyl, wherein the 4-7 membered heterocycloalkyl of R^{1c} is optionally substituted with a substituent selected from the group consisting of —C₁₋₆-alkyl-NH₂ and —C₁₋₆-alkyl-C(O)OH;
or A¹ is a moiety of formula (II):

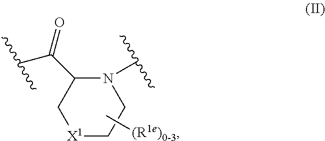

wherein:
X¹ is absent, O, NH, CH₂, or CHR^{1e}; and
each R^{1e} is independently selected from C₁₋₃-alkyl, —NH₂, —NHC₁₋₆-alkyl, —N(C₁₋₆-alkyl)₂, —OH, halo, and C(O)R^{1d}, wherein R^{1d} is selected from the group consisting of H and —C₁₋₆-alkyl.

In further embodiments, A¹ is

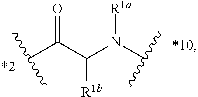

wherein
R^{1a} is H or CH₃;
R^{1b} is selected from the group consisting of H and C₁₋₆-alkyl, wherein the C₁₋₆-alkyl of R^{1b} is optionally substituted with a substituent selected from —C(O)OH, —OH, —NH₂, —NHC₁₋₆-alkyl, —N(C₁₋₆-alkyl)₂, and —NH—C(O)R^{1c}; and
R^{1c} is a 4-7 membered heterocycloalkyl, wherein the 4-7 membered heterocycloalkyl of R^{1c} is optionally substituted with a substituent selected from the group consisting of —C₁₋₆-alkyl-NH₂ and —C₁₋₆-alkyl-C(O)OH.

In further embodiments, A¹ is

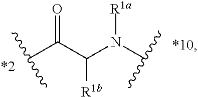

wherein
R^{1a} is H or CH₃;
R^{1b} is selected from the group consisting of H and C₁₋₆-alkyl, wherein the C₁₋₆-alkyl of R^{1b} is optionally substituted with a substituent selected from —C(O)OH, —OH, —NH₂, —NHC₁₋₆-alkyl, —N(C₁₋₆-alkyl)₂, and —NH—C(O)R^{1c}; and
R^{1c} is a 6 membered heterocycloalkyl optionally substituted with a substituent selected from the group consisting of —C₁₋₆-alkyl-NH₂ and —C₁₋₆-alkyl-C(O)OH.

In further embodiments, A¹ is a moiety of formula (III):

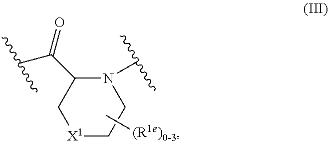

wherein:
X¹ is absent, O, NH, CH₂, or CHR^{1e}; and
each R^{1e} is independently selected from C₁₋₃-alkyl, —NH₂, —NHC₁₋₆-alkyl, —N(C₁₋₆-alkyl)₂, —OH, halo, and C(O)R^{1d}, wherein R^{1d} is selected from the group consisting of H and —C₁₋₆-alkyl.

In further embodiments, A¹ is a moiety of formula (II):

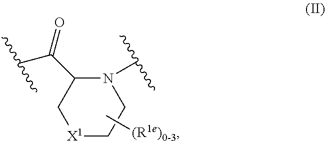

wherein:
X¹ is absent; and
each R^{1e} is independently selected from —OH, —NH₂, and —C₁₋₆-alkyl.

In still further embodiments, A¹ is a moiety of formula (II):

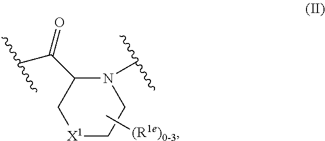

wherein:
X¹ is absent; and
each R^{1e} is independently selected from —OH, and —C₁₋₃-alkyl.

In further embodiments, A¹ is selected from the group consisting of:

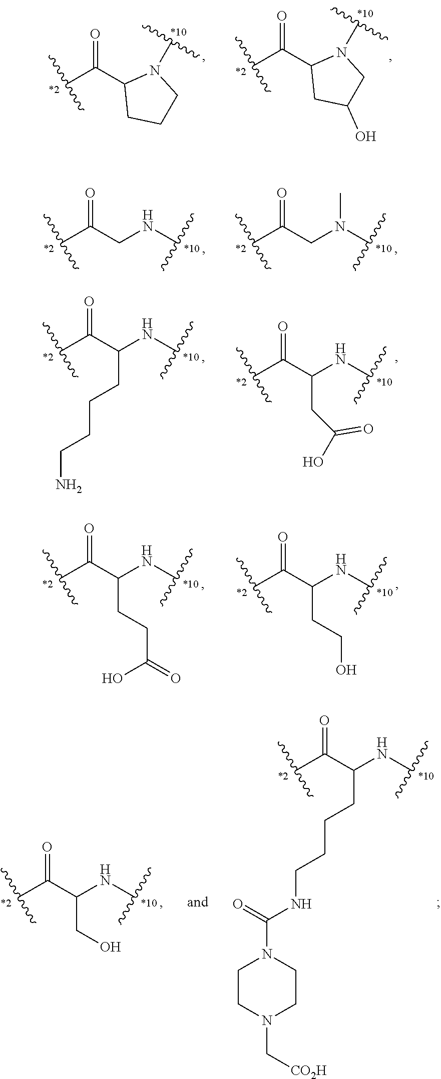

wherein
*2 indicates the point of attachment to A² and *10 indicates the point of attachment to A¹⁰

In further embodiments, A¹ is selected from the group consisting of:

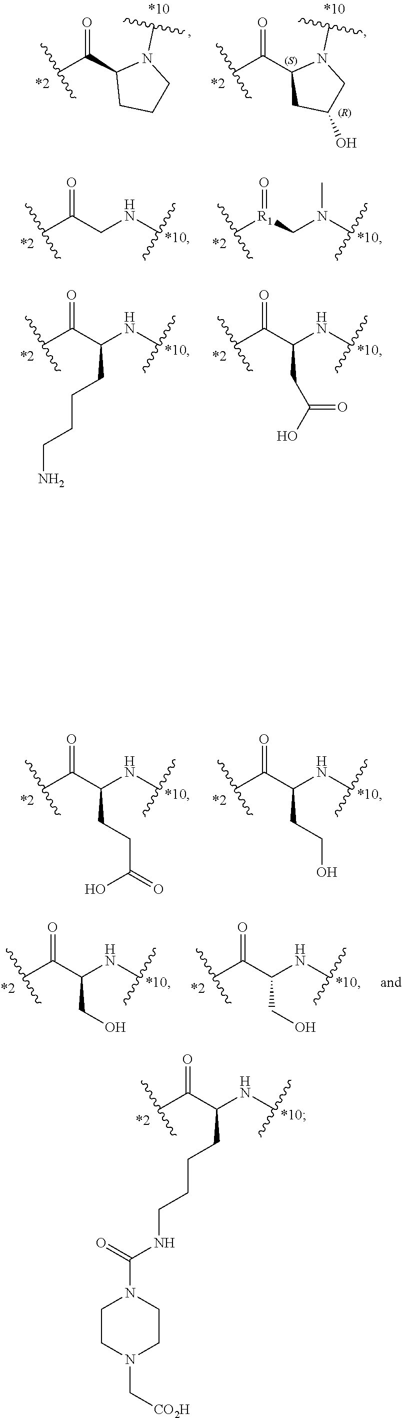

wherein
*2 indicates the point of attachment to A² and *10 indicates the point of attachment to A¹⁰

In further embodiments, A¹ is

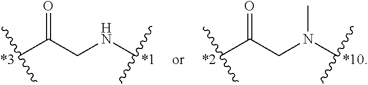

In further embodiments, A¹ is

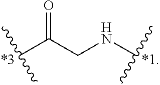 [SMILES: *NCC(*)=O]

In further embodiments, A¹ is

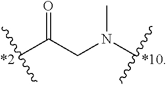 [SMILES: *C(=O)CN(*)C]

In further embodiments, A¹ is

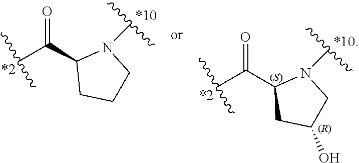

In further embodiments, A¹ is

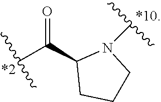 [SMILES: *C(=O)[C@@H]1CCCN1*]

In further embodiments, A¹ is

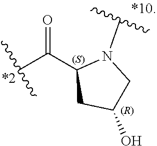

In further embodiments, A² is

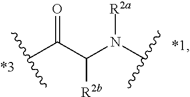

wherein
R^{2a} is selected from the group consisting of H and C₁₋₃-alkyl;
R^{2b} is selected from the group consisting of H and C₁₋₆-alkyl, wherein the C₁₋₆-alkyl of R^{2b} is optionally substituted with 1, 2, 3, or 4 substituents independently selected from the group consisting of —C(O)OH, —OH, —NH₂, —NHC₁₋₆-alkyl, and —N(C₁₋₆-alkyl)₂; and
*1 indicates the point of attachment to A¹ and *3 indicates the point of attachment to A³.

In some embodiments, A² is

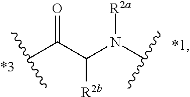

wherein
R^{2a} is H or CH₃;
R^{2b} is selected from the group consisting of H and C₁₋₆-alkyl, wherein the C₁₋₆-alkyl of R^{2b} is optionally substituted with a substituent selected from —C(O)OH, —OH, —NH₂, —NHC₁₋₆-alkyl, and —N(C₁₋₆-alkyl)₂.

In further embodiments, A² is

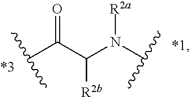

wherein
R^{2a} is H;
R^{2b} is selected from the group consisting of H and C₁₋₆-alkyl, wherein the C₁₋₆-alkyl of R^{2b} is optionally substituted with a substituent selected from —C(O)OH, and —OH.

In further embodiments, A² is selected from the group consisting of:

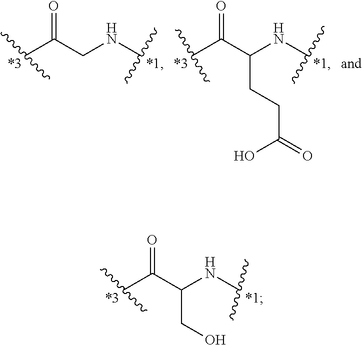 [SMILES: *NCC(*)=O ; *NC(CCC(=O)O)C(*)=O ; *NC(CO)C(*)=O]

wherein *1 indicates the point of attachment to A¹ and *3 indicates the point of attachment to A³.

In further embodiments, A² is selected from the group consisting of:

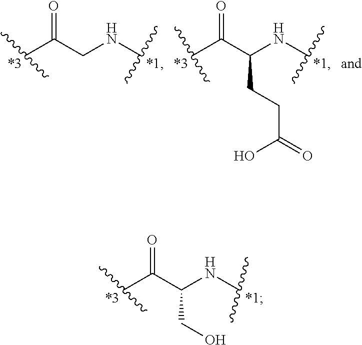 [SMILES: *NCC(*)=O ; *N[C@@H](CCC(=O)O)C(*)=O ; *N[C@H](CO)C(*)=O]

wherein *1 indicates the point of attachment to A¹ and *3 indicates the point of attachment to A³.

In further embodiments, A² is

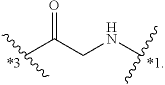 [SMILES: *NCC(*)=O]

In further embodiments, A² is

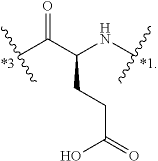 [SMILES: *N[C@@H](CCC(=O)O)C(*)=O]

In further embodiments, A² is

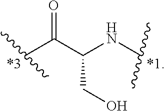 [SMILES: *N[C@H](CO)C(*)=O]

In further embodiments, A³ is

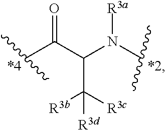

wherein
R^{3a} is selected from the group consisting of H and C₁₋₃-alkyl;
R^{3b} is selected from the group consisting of H, C₁₋₃-alkyl, and halo;
R^{3c} is selected from the group consisting of halo, C₁₋₆ alkyl, C₃₋₈-cycloalkyl, phenyl, and 5-6 membered heteroaryl, wherein the C₁₋₆ alkyl, phenyl, and 5-6 membered heteroaryl of R^{3c} are optionally substituted with 1, 2, 3, or 4 substituents independently selected from the group consisting of halo, —C(O)OH, —C(O)C₁₋₆-alkyl, —OH, —NHC₁₋₆-alkyl, —N(C₁₋₆-alkyl)₂, and —NH₂;
R^{3d} is selected from the group consisting of H, C₁₋₃-alkyl, and halo; and
*2 indicates the point of attachment to A² and *4 indicates the point of attachment to A⁴.

In further embodiments, A³ is

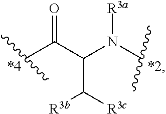

wherein
R^{3a} is H and CH₃;
R^{3b} is selected from the group consisting of H and C₁₋₃-alkyl; and
R^{3c} is selected from the group consisting of C₁₋₆ alkyl, C₁₋₆-haloalkyl, C₃₋₈-cycloalkyl, phenyl, and 5-6 membered heteroaryl, wherein the phenyl and 5-6 membered heteroaryl of R^{3c} are optionally substituted with halo, —C(O)OH, —C(O)C₁₋₆-alkyl, —OH, —NHC₁₋₆-alkyl, —N(C₁₋₆-alkyl)₂, and —NH₂.

In some embodiments, A³ is selected from the group consisting of:

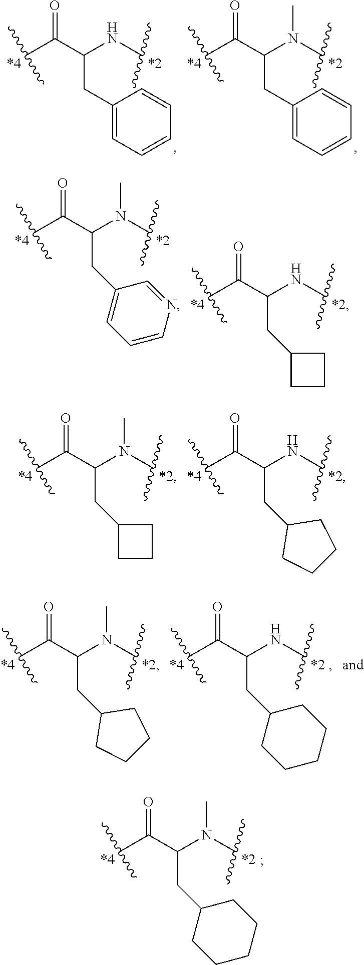

wherein *2 indicates the point of attachment to A² and *4 indicates the point of attachment to A⁴.

In further embodiments, A³ is selected from the group consisting of:

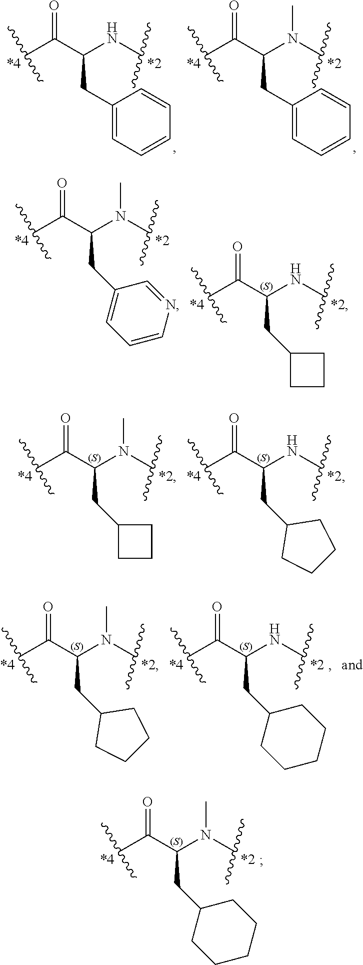

wherein *2 indicates the point of attachment to A² and *4 indicates the point of attachment to A⁴.

In further embodiments, A³ is

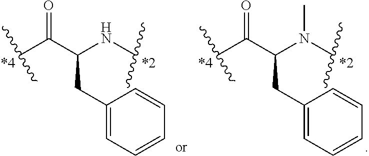 [SMILES: *N[C@@H](Cc1ccccc1)C(*)=O ; *C(=O)[C@H](Cc1ccccc1)N(*)C]

In further embodiments, A³ is

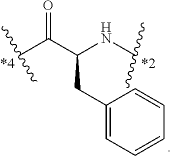 [SMILES: *N[C@@H](Cc1ccccc1)C(*)=O]

In further embodiments, A³ is

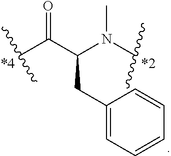 [SMILES: *C(=O)[C@H](Cc1ccccc1)N(*)C]

In further embodiments, A³ is

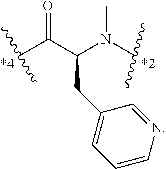 [SMILES: *C(=O)[C@H](Cc1cccnc1)N(*)C]

In further embodiments, A³ is

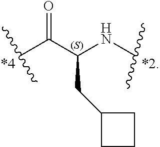

In further embodiments, A³ is

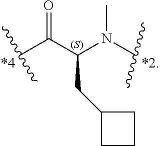

In further embodiments, A³ is

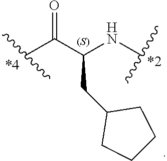

In further embodiments, A³ is

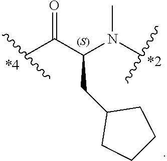

In further embodiments, A³ is

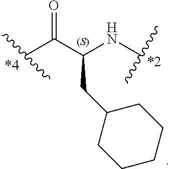

In further embodiments, A³ is

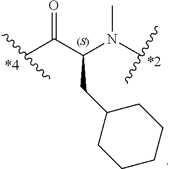

In further embodiments, A⁴ is

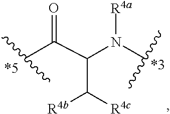

wherein
R^{4a} is selected from the group consisting of H and C₁₋₃-alkyl;
R^{4b} is selected from the group consisting of H and C₁₋₃-alkyl;
R^{4c} is selected from the group consisting of phenyl and 5-6 membered heteroaryl wherein phenyl and 5-6 membered heteroaryl of R⁴ are optionally substituted with 1, 2, 3, 4, or 5 substituents R^{4d};
each R^{4d} is independently selected from C₁₋₆-alkyl, —OH, —OC₁-C₆-alkyl, —NH₂, halo, —NHC(O)R^{4f}, —C(O)NHR^{4f}, and —C(O)OR^{4f}, wherein the C₁₋₆-alkyl and —OC₁-C₆-alkyl of R^{4d} are each optionally substituted with 1, 2, or 3 substituents R^{4e};
each R^{4e} is independently selected from —CN, —NH₂, —NHC₁₋₆-alkyl, —N(C₁₋₆-alkyl)₂, —NHC(O)—(C₁₋₆-alkyl), —N₃, —OH, —C(O)OH, —C(O)NH₂, and 5-6 membered heteroaryl, wherein the 5-6 membered heteroaryl of R⁴ is optionally substituted with —C₁₋₆-alkyl-NH₂ or —C₁₋₃-alkyl-OH;
each R^{4f} is independently selected from the group consisting of H and C₁₋₆-alkyl; and
*3 indicates the point of attachment to A³ and *5 indicates the point of attachment to A⁵.

In further embodiments, A⁴ is

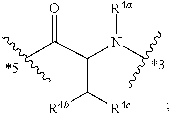

wherein
R^{4a} is H or CH₃;
R^{4b} is selected from the group consisting of H and C₁₋₃-alkyl,
R^{4c} is selected from the group consisting of phenyl and 5-6 membered heteroaryl of R⁴ wherein phenyl and 5-6 membered heteroaryl are substituted with a substituent R^{4d},
R^{4d} is selected from C₁₋₆-alkyl, —OH, and —OC₁-C₆-alkyl, wherein the C₁₋₆-alkyl and —OC₁-C₆-alkyl of R^{4d} are each optionally substituted with a substituent R^{4c}, and
R^{4e} is selected from —NH₂, —NHC₁₋₆-alkyl, —N(C₁₋₆-alkyl)₂, and 5-6 membered heteroaryl, wherein the 5-6 membered heteroaryl of R⁴ is optionally substituted with —C₁₋₆-alkyl-NH₂ or —C₁₋₃-alkyl-OH.

In further embodiments, A⁴ is

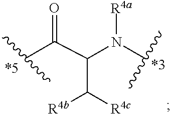

wherein
R^{4a} is H or CH₃;
R^{4b} is selected from the group consisting of H and C₁₋₃-alkyl,
R^{4c} is phenyl substituted with a substituent R^{4d},
R^{4d} is selected from C₁₋₆-alkyl, —OH, and —OC₁-C₆-alkyl, wherein the C₁₋₆-alkyl and —OC₁-C₆-alkyl of R^{4d} are each optionally substituted with a substituent R^{4e},
R^{4e} is selected from —NH₂ and 5-6 membered heteroaryl, wherein the 5-6 membered heteroaryl is optionally substituted with —C₁₋₆-alkyl-NH₂.

In further embodiments, A⁴ is a moiety of formula (III):

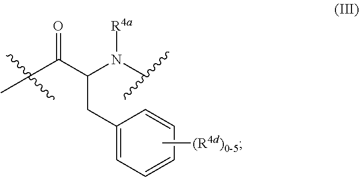

wherein:
R^{4a} is selected from the group consisting of H and C₁₋₃-alkyl,
each R^{4d} is independently selected from the group consisting of —C₁₋₆-alkyl, —OH, —OC₁₋₆-alkyl, —NH₂, halo, —NHC(O)R^{4f}, —C(O)NHR^{4f}, and —C(O)OR^{4f}, wherein the C₁₋₆-alkyl and —OC₁₋₆-alkyl of R^{4d} are each optionally substituted with 1, 2, or 3 substituents independently selected from the group consisting of —CN, —NH₂, —NHC₁₋₆-alkyl, —N(C₁₋₆-alkyl)₂, N₃, —OH, —C(O)OH, —C(O)NH₂, and 5-6 membered heteroaryl optionally substituted with —C₁₋₃-alkyl-NH₂ or —C₁₋₃-alkyl-OH; and
each R^{4f} is independently selected from the group consisting of H and C₁₋₆-alkyl.

In further embodiments, A⁴ is a moiety of formula (III):

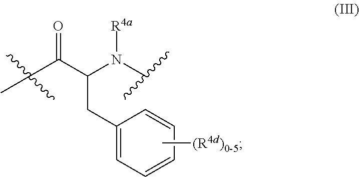

wherein:
R^{4a} is selected from the group consisting of H and CH₃; and
each R^{4d} is independently selected from, —OH, —OC₁₋₆-alkyl, —NH₂, —NHC(O)R^{4f}, —C(O)NHR⁴, and —C₁₋₆-alkyl, wherein the —OC₁₋₆-alkyl and —C₁₋₆-alkyl of R^{4d} are optionally substituted with —NH₂ or 5-6 membered heteroaryl, wherein the 5-6 membered heteroaryl is optionally substituted with —C₁₋₃-alkyl-NH₂ or —C₁₋₃-alkyl-OH.

In further embodiments, A⁴ is a moiety of formula (III):

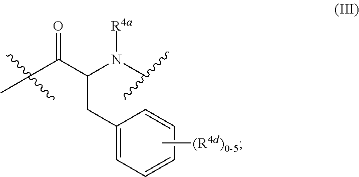

wherein:
R^{4a} is selected from the group consisting of H; and
each R^{4d} is independently selected from —OH, —OC₁₋₆-alkyl, —C₁₋₆-alkyl, wherein the —OC₁₋₆-alkyl and —C₁₋₆-alkyl of R^{4d} are optionally substituted with —NH₂ or 5-6 membered heteroaryl, wherein the 5-6 membered heteroaryl is optionally substituted with —C₁₋₃-alkyl-NH₂.

In further embodiments, A⁴ is selected from the group consisting of:

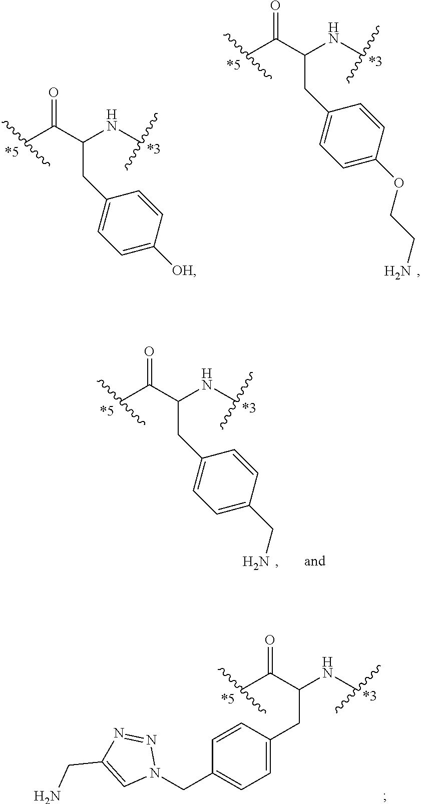 [SMILES: *NC(Cc1ccc(O)cc1)C(*)=O ; *NC(Cc1ccc(OCCN)cc1)C(*)=O ; *NC(Cc1ccc(CN)cc1)C(*)=O ; *NC(Cc1ccc(Cn2cc(CN)nn2)cc1)C(*)=O]

wherein *3 indicates the point of attachment to A³ and *5 indicates the point of attachment to A⁵.

In further embodiments, A⁴ is selected from the group consisting of:

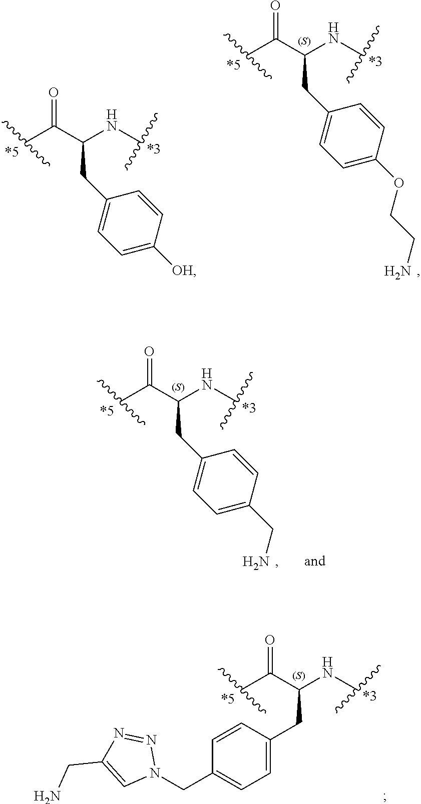

wherein *3 indicates the point of attachment to A³ and *5 indicates the point of attachment to A⁵.

In further embodiments, A⁴ is

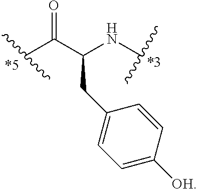 [SMILES: *N[C@@H](Cc1ccc(O)cc1)C(*)=O]

In further embodiments, A⁴ is

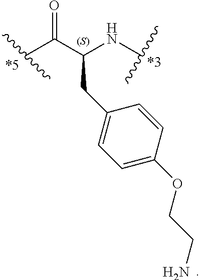

In further embodiments, A⁴ is

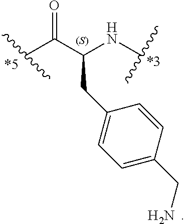

In further embodiments, A⁴ is

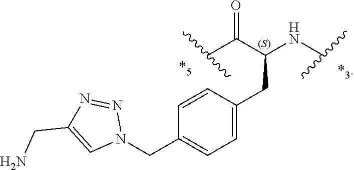

In further embodiments, A⁵ is

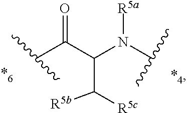

wherein
R^{5a} is selected from the group consisting of H and C₁₋₃-alkyl,
R^{5b} is selected from the group consisting of H, C₁₋₃-alkyl, and —OH,
R^{5c} is selected from the group consisting of H and C₁₋₆-alkyl, wherein the C₁₋₆-alkyl of R^{5c} is optionally substituted with halo, —OH, —OC₁₋₆-alkyl, and —C(O)R^{5d},
R^{5d} is 4-7 membered heterocycloalkyl optionally substituted with 1, 2, 3, or 4 groups independently selected from C₁₋₃-alkyl, —CN, halo, and —OH, and
*4 indicates the point of attachment to A⁴ and *6 indicates the point of attachment to A⁶.

In further embodiments, A⁵ is

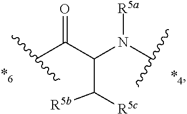

wherein
R^{5a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, and —C(O)R^{5d};
R^{5d} is 4-7 membered heterocycloalkyl optionally substituted with 1, 2, 3, or 4 groups independently selected from C₁₋₃-alkyl, —CN, halo, and —OH; and
wherein *4 indicates the point of attachment to A⁴ and *6 indicates the point of attachment to A⁶.

In further embodiments, A⁵ is

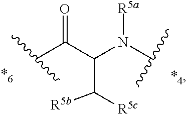

wherein
R^{5a} is H or CH₃;
R^{5b} is selected from the group consisting of H, and —OH,
R^{5c} is selected from the group consisting of H and C₁₋₈-alkyl, wherein the C₁₋₈-alkyl of R^{5c} is optionally substituted with —OH, —OC₁₋₆-alkyl, and —C(O)R^{5d},
R^{5d} is 5 membered heterocycloalkyl optionally substituted with 1, 2, or 3 groups independently selected from —CN, halo, and —OH.

In further embodiments, A⁵ is selected from the group consisting of

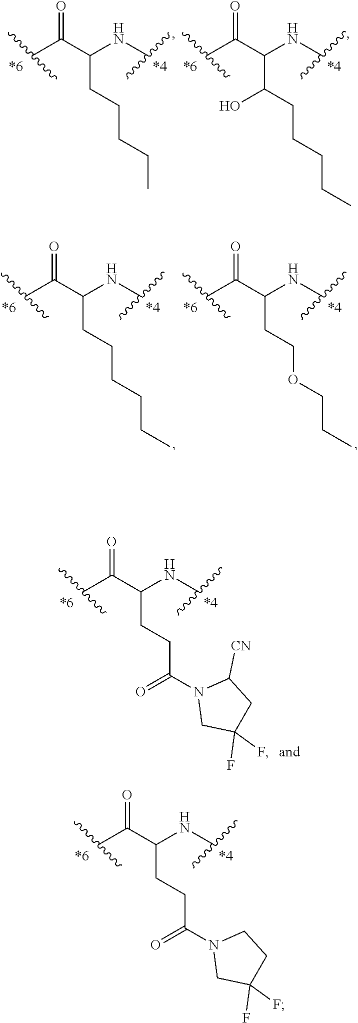 [SMILES: *NC(CCCCC)C(*)=O ; *NC(C(*)=O)C(O)CCCCC ; *NC(CCCCCC)C(*)=O ; *NC(CCOCCC)C(*)=O ; *NC(CCC(=O)N1CC(F)(F)CC1C#N)C(*)=O ; *NC(CCC(=O)N1CCC(F)(F)C1)C(*)=O]

wherein *4 indicates the point of attachment to A⁴ and *6 indicates the point of attachment to A⁶.

In further embodiments, A⁵ is selected from the group consisting of

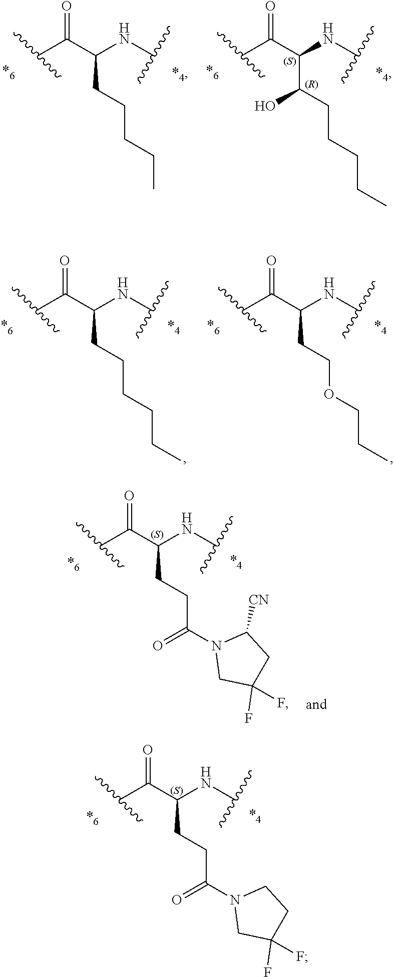

wherein *4 indicates the point of attachment to A⁴ and *6 indicates the point of attachment to A⁶.

In further embodiments, A⁵ is

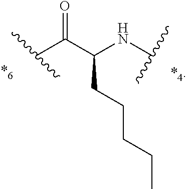 [SMILES: *N[C@@H](CCCCC)C(*)=O]

In further embodiments, A⁵ is

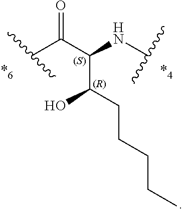

In further embodiments, A⁵ is

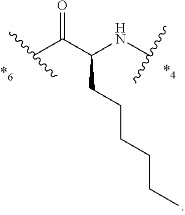 [SMILES: *N[C@@H](CCCCCC)C(*)=O]

In further embodiments, A⁵ is

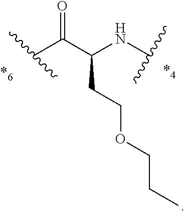 [SMILES: *N[C@@H](CCOCCC)C(*)=O]

In further embodiments, A⁵ is

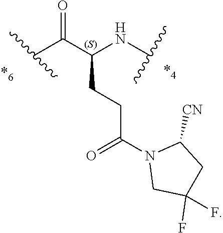

In further embodiments, A⁵ is

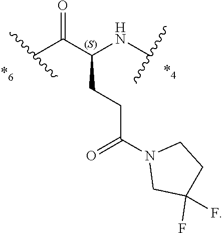

In further embodiments, A⁶ is

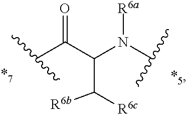

wherein
R^{6a} is selected from the group consisting of H and C₁₋₃-alkyl,
R^{6b} is selected from the group consisting of H and C₁₋₃-alkyl,
R^{6c} is a 5 to 10 membered heteroaryl optionally substituted with 1, 2, 3, or 4 groups independently selected from C₁₋₃-alkyl, —CN, halo, —NH₂, —NHC₁₋₆-alkyl, —N(C₁₋₆-alkyl)₂, —OH, and —OC₁₋₆-alkyl, and
*5 indicates the point of attachment to A⁵ and *7 indicates the point of attachment to A⁷.

In further embodiments, A⁶ is

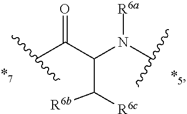

wherein
R^{6a} is H or CH₃;
R^{6b} is selected from the group consisting of H and C₁₋₃-alkyl,
R^{6c} is a 5 to 10 membered heteroaryl optionally substituted with a group selected from C₁₋₃-alkyl, halo, —NH₂, —NHC₁₋₆-alkyl, —N(C₁₋₆-alkyl)₂, —OH, and —OC₁₋₃-alkyl.

In further embodiments, A⁶ is

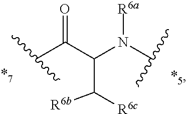

wherein
R^{6a} is H or CH₃;
R^{6b} is selected from the group consisting of H and C₁₋₃-alkyl,
R^{6c} is an 8 to 10 membered heteroaryl optionally substituted with a group selected from C₁₋₃-alkyl, halo, —OH, and —OC₁₋₃-alkyl.

In further embodiments, A⁶ is selected from the group consisting of:

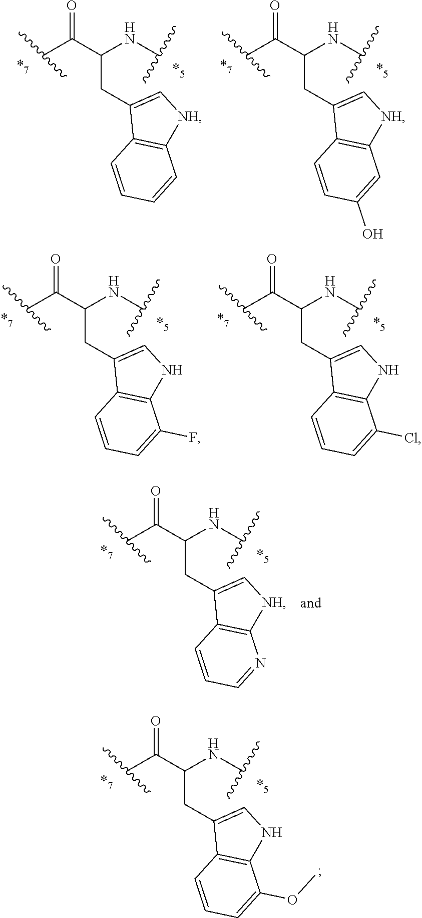

wherein *5 indicates the point of attachment to A⁵ and
*7 indicates the point of attachment to A⁷.

In further embodiments, A⁶ is selected from the group consisting of:

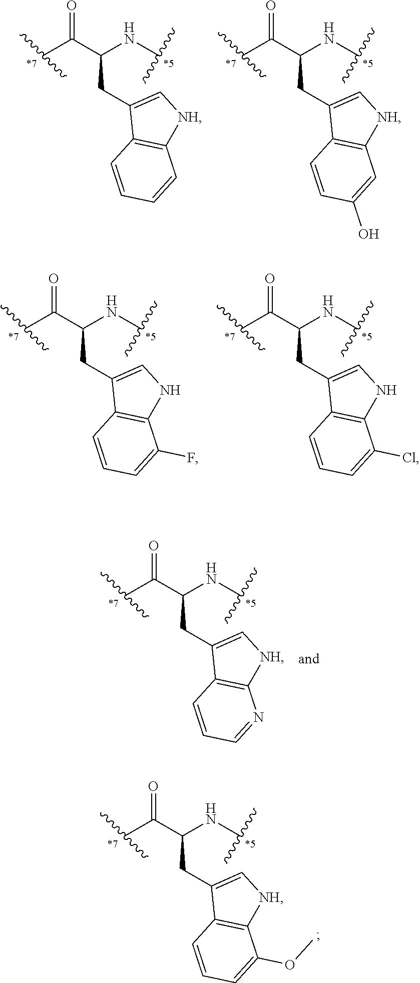 [SMILES: *N[C@@H](Cc1c[nH]c2ccccc12)C(*)=O ; *N[C@@H](Cc1c[nH]c2cc(O)ccc12)C(*)=O ; *N[C@@H](Cc1c[nH]c2c(F)cccc12)C(*)=O ; *N[C@@H](Cc1c[nH]c2c(Cl)cccc12)C(*)=O ; *N[C@@H](Cc1c[nH]c2ncccc12)C(*)=O ; *N[C@@H](Cc1c[nH]c2c(OC)cccc12)C(*)=O]

wherein *5 indicates the point of attachment to A⁵ and *7 indicates the point of attachment to A⁷.

In further embodiments, A⁶ is

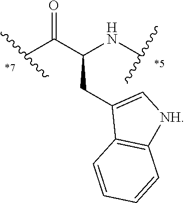 [SMILES: *N[C@@H](Cc1c[nH]c2ccccc12)C(*)=O]

In further embodiments, A⁶ is

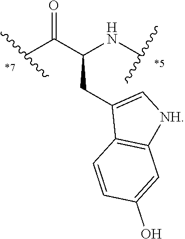 [SMILES: *N[C@@H](Cc1c[nH]c2cc(O)ccc12)C(*)=O]

In further embodiments, A⁶ is

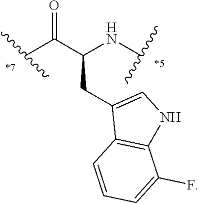 [SMILES: *N[C@@H](Cc1c[nH]c2c(F)cccc12)C(*)=O]

In further embodiments, A⁶ is

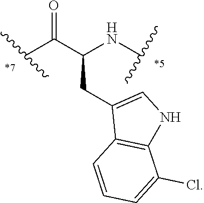 [SMILES: *N[C@@H](Cc1c[nH]c2c(Cl)cccc12)C(*)=O]

In further embodiments, A⁶ is

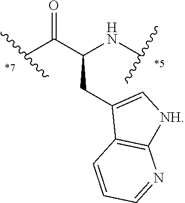 [SMILES: *N[C@@H](Cc1c[nH]c2ncccc12)C(*)=O]

In further embodiments, A⁶ is

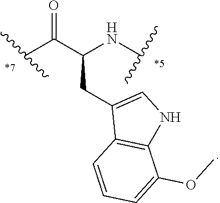 [SMILES: *N[C@@H](Cc1c[nH]c2c(OC)cccc12)C(*)=O]

In further embodiments, A⁷ is

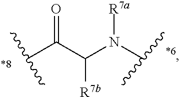

wherein
R^{7a} is selected from the group consisting of H and C₁₋₃-alkyl,
RV is selected from the group consisting of H and C₁₋₆-alkyl, and
*6 indicates the point of attachment to A⁶ and *8 indicates the point of attachment to A⁸.

In further embodiments, A⁷ is

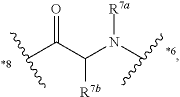

wherein
R^{7a} is H or CH₃;
R^{7b} is selected from the group consisting of H and C₁₋₃-alkyl.

In further embodiments, A⁷ is

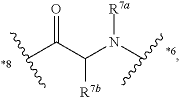

wherein
R^{7a} is H;
R is H or CH₃.

In further embodiments, A⁷ is

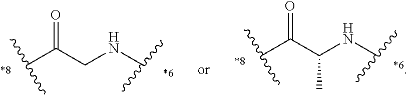

In further embodiments, A⁷ is

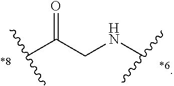 [SMILES: *NCC(*)=O]

In further embodiments, A⁷ is

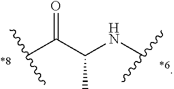 [SMILES: *N[C@H](C)C(*)=O]

In further embodiments, A⁸ is

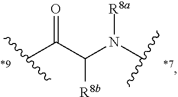

wherein
R^{8a} is selected from the group consisting of H, C₁₋₃-alkyl, and —CH₂-phenyl, wherein the —CH₂-phenyl of R^{8a} is optionally substituted with —NH₂, —NHC₁₋₆-alkyl, —N(C₁₋₆-alkyl)₂, —OH, —OC₁₋₃-alkyl, or C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), and —C₁₋₆-alkyl-NH—C(O)R^{8c}, wherein C₁₋₆-alkyl, —C₁₋₆-alkyl-phenyl, and —C₁₋₆-alkyl(5-6 membered heteroaryl) of R^{8b} are optionally substituted with halo, —NHC₁₋₆-alkyl, —N(C₁₋₆-alkyl)₂, —NH₂, —OH, —OC₁₋₃-alkyl, or C₁₋₃-alkyl;
R^{8c} is a 4-7 membered heterocycloalkyl optionally substituted with —C₁₋₆-alkylC(O)OH or —C(O)OH, and
*7 indicates the point of attachment to A⁷ and *9 indicates the point of attachment to A⁹.

In further embodiments, A⁸ is

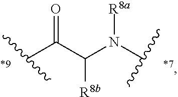

wherein
R^{8a} is selected from the group consisting of H, CH₃, and —CH₂-phenyl, wherein the —CH₂-phenyl of R^{8a} is optionally substituted with —NH₂, —NHC₁₋₆-alkyl, —N(C₁₋₆-alkyl)₂, —OH, —OC₁₋₃-alkyl, or C₁₋₃-alkyl;
R^{8b} is selected from the group consisting of H, C₁₋₆-alkyl, —CH₂-phenyl, —CH₂-(5-6 membered heteroaryl), and —C₁₋₆-alkyl-NH—C(O)R^{8c}, wherein C₁₋₆-alkyl, —CH₂-phenyl, and —CH₂-(5-6 membered heteroaryl) of R^{8b} are optionally substituted with halo, —NHC₁₋₆-alkyl, —N(C₁₋₆-alkyl)₂, —NH₂, —OH, —OC₁₋₃-alkyl, or C₁₋₃-alkyl;
R^{8c} is a 4-7 membered heterocycloalkyl optionally substituted with —C₁₋₆-alkylC(O)OH.

In further embodiments, A⁸ is

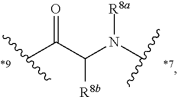

wherein
R^{8a} is selected from the group consisting of H, CH₃, and —CH₂-phenyl;
R^{8b} is selected from the group consisting of H, C₁₋₆-alkyl, —CH₂-phenyl, —CH₂-(5-6 membered heteroaryl), wherein C₁₋₆-alkyl, —CH₂-phenyl, and —CH₂-(5-6 membered heteroaryl) of R^{8b} are optionally substituted with halo, —NHC₁₋₆-alkyl, —N(C₁₋₆-alkyl)₂, —NH₂, —OH, —OCH₃, or C₁₋₃-alkyl;
or A⁸ is a moiety of formula (IV):

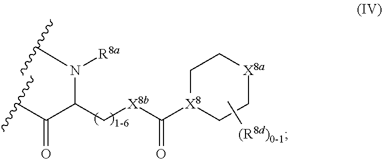

wherein:
X⁸ is selected from the group consisting of N and CH;
X^{8a} is selected from the group consisting of NH, NR^{8d}, O, CH₂, and CHR^{8d};
X^{8b} is selected from the group consisting of NH, NR^{8a}, O, and CH₂;
R^{8a} is selected from the group consisting of H and C₁₋₃-alkyl; and
R^{8d} is selected from the group consisting of —C₁₋₆-alkylC(O)OH and —C(O)OH;
wherein at least one of X⁸ and X^{8a} is N.

In further embodiments, A⁸ is

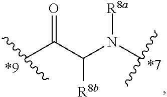

wherein
R^{8a} is selected from the group consisting of H, CH₃, and —CH₂-phenyl;
R^{8b} is selected from the group consisting of H, C₁₋₆-alkyl, —CH₂-phenyl, —CH₂-(5 to 6 membered heteroaryl), wherein C₁₋₆-alkyl, —CH₂-phenyl, and —CH₂-(5 to 6 membered heteroaryl) of R^{8b} are optionally substituted with halo, —NHC₁₋₆-alkyl, —N(C₁₋₆-alkyl)₂, —NH₂, —OH, —OCH₃, or C₁₋₃-alkyl.

In further embodiments, A⁸ is a moiety of formula (IV):

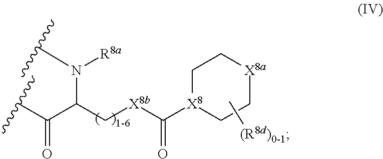

wherein:
X⁸ is selected from the group consisting of N and CH;
X^{8a} is selected from the group consisting of NR^{8d}, O, CH₂, and CHR^{8d};
X^{8b} is selected from the group consisting of NH, NR^{8a}, O, and CH₂;
R^{8a} is selected from the group consisting of H and C₁₋₃-alkyl; and
R^{8d} is selected from the group consisting of —C₁₋₆-alkylC(O)OH and —C(O)OH;
wherein at least one of X⁸ and X^{8a} is N.

In further embodiments, A⁸ is a moiety of formula (IV):

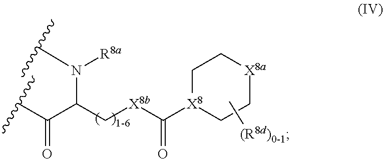

wherein:
X⁸ is selected from the group consisting of N and CH;
X^{8a} is selected from the group consisting of NR^{8d}, O, CH₂, and CHR^{8d};
X^{8b} is selected from the group consisting of NH, NR^{8a}, O, and CH₂;
R^{8a} is selected from the group consisting of H and CH₃; and
R^{8d} is —C₁₋₆-alkylC(O)OH;
wherein at least one of X⁸ and X^{8a} is N.

In further embodiments, A⁸ is selected from the group consisting of:

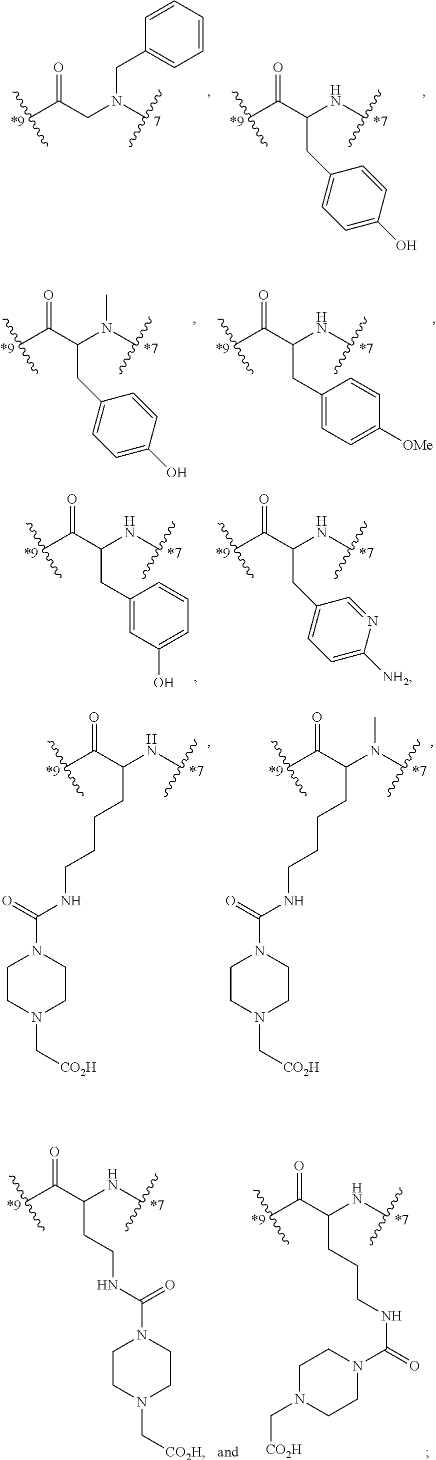

wherein *7 indicates the point of attachment to A⁷ and *9 indicates the point of attachment to A⁹.

In further embodiments, A⁸ is selected from the group consisting of:

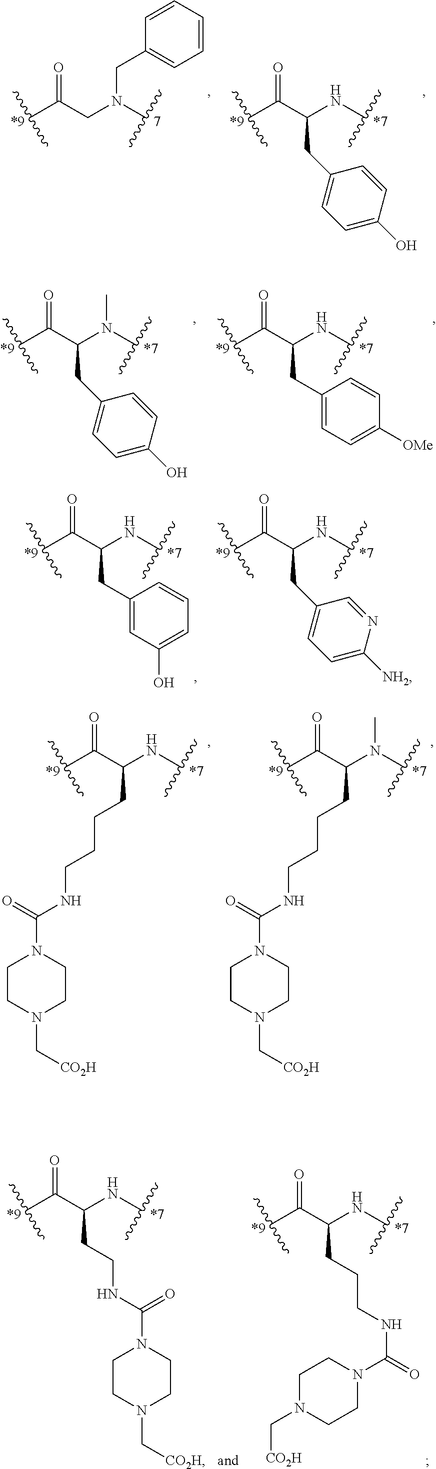

wherein *7 indicates the point of attachment to A⁷ and *9 indicates the point of attachment to A⁹.

In further embodiments, A⁸ is

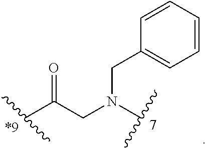 [SMILES: *C(=O)CN(*)Cc1ccccc1]

In further embodiments, A⁸ is

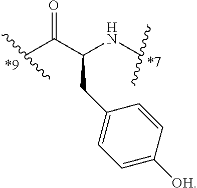 [SMILES: *N[C@@H](Cc1ccc(O)cc1)C(*)=O]

In further embodiments, A⁸ is

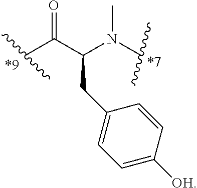 [SMILES: *C(=O)[C@H](Cc1ccc(O)cc1)N(*)C]

In further embodiments, A⁸ is

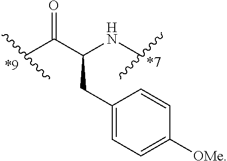 [SMILES: *N[C@@H](Cc1ccc(OC)cc1)C(*)=O]

In further embodiments, A⁸ is

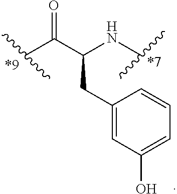 [SMILES: *N[C@@H](Cc1cccc(O)c1)C(*)=O]

In further embodiments, A⁸ is

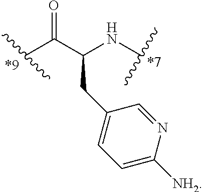 [SMILES: *N[C@@H](Cc1ccc(N)nc1)C(*)=O]

In further embodiments, A⁸ is

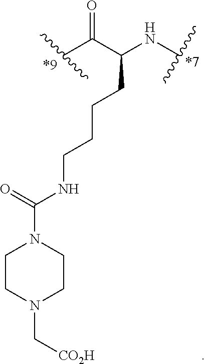 [SMILES: *N[C@@H](CCCCNC(=O)N1CCN(CC(=O)O)CC1)C(*)=O]

In further embodiments, A⁸ is

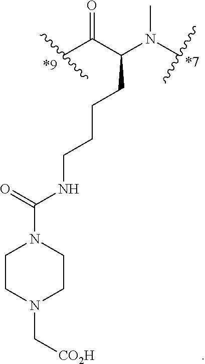 [SMILES: *C(=O)[C@H](CCCCNC(=O)N1CCN(CC(=O)O)CC1)N(*)C]

In further embodiments, A⁸ is

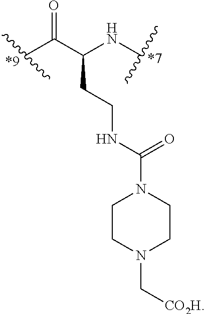 [SMILES: *N[C@@H](CCNC(=O)N1CCN(CC(=O)O)CC1)C(*)=O]

In further embodiments, A⁸ is

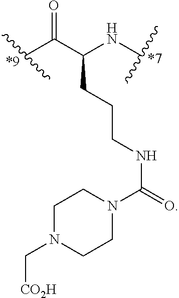 [SMILES: *N[C@@H](CCCNC(=O)N1CCN(CC(=O)O)CC1)C(*)=O]

In some embodiments, A⁹ is

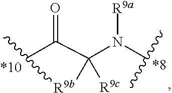

wherein
R^{9a} is selected from the group consisting of H and C₁₋₃-alkyl;
R^{9b} is selected from the group consisting of H and C₁₋₃-alkyl;
R^{9c} is C₁₋₆-alkyl optionally substituted with 1, 2, 3, or 4 groups independently selected from —CN, —C(O)OH, —C(O)NH₂, halo, —NH₂, —NHC₁₋₆-alkyl, —N(C₁₋₆-alkyl)₂, —OH, and —OC₁₋₆-alkyl; and
*8 indicates the point of attachment to A⁸ and *10 indicates the point of attachment to A¹⁰.

In further embodiments, A⁹ is

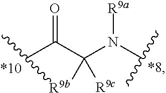

wherein
R^{9a} is H or CH₃;
R^{9b} is selected from the group consisting of H and C₁₋₃-alkyl; and
R^{9c} is C₁₋₆-alkyl optionally substituted with a group selected from —C(O)OH, —C(O)NH₂, —NH₂, —NHC₁₋₆-alkyl, —N(C₁₋₆-alkyl)₂, —OH, and —OC₁₋₆-alkyl.

In further embodiments, A⁹ is

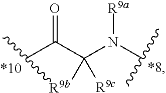

wherein
R^{9a} is H or CH₃;
R^{9b} is H or CH₃; and
R^{9c} is C₁₋₆-alkyl optionally substituted with a group selected from —C(O)OH, —C(O)NH₂, —NH₂, —NHC₁₋₆-alkyl, —N(C₁₋₆-alkyl)₂, and —OH.

In further embodiments, A⁹ is selected from the group consisting of:

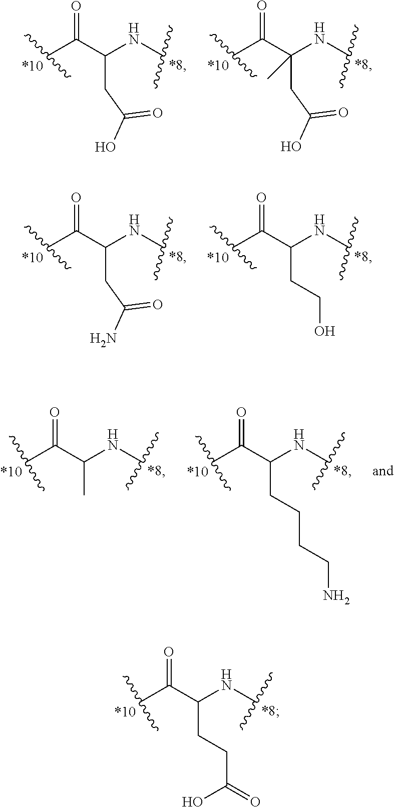

wherein *8 indicates the point of attachment to A⁸ and *10 indicates the point of attachment to A¹⁰.

In further embodiments, A⁹ is selected from the group consisting of:

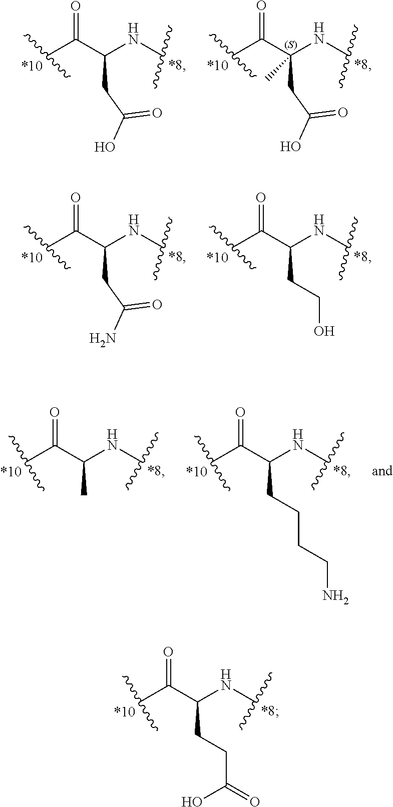

wherein *8 indicates the point of attachment to A⁸ and *10 indicates the point of attachment to A¹⁰.

In further embodiments, A⁹ is

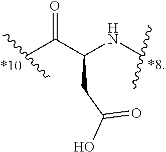 [SMILES: *N[C@@H](CC(=O)O)C(*)=O]

In further embodiments, A⁹ is

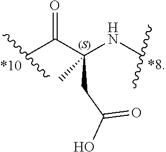

In further embodiments, A⁹ is

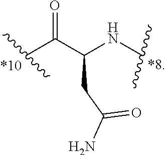 [SMILES: *N[C@@H](CC(N)=O)C(*)=O]

In further embodiments, A⁹ is

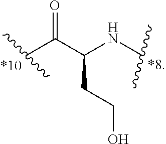 [SMILES: *N[C@@H](CCO)C(*)=O]

In further embodiments, A⁹ is

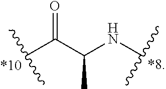 [SMILES: *N[C@@H](C)C(*)=O]

In further embodiments, A⁹ is

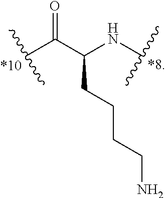 [SMILES: *N[C@@H](CCCCN)C(*)=O]

In further embodiments, A⁹ is

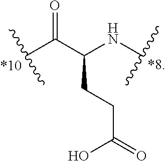 [SMILES: *N[C@@H](CCC(=O)O)C(*)=O]

In some embodiments, A¹⁰ is

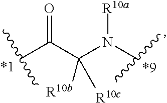

wherein
R^{10a} is selected from the group consisting of H and C₁₋₃-alkyl;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents selected from —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1, 2, 3, or 4 substituents independently selected from C₁₋₃-alkyl, —NH₂, —NHC₁₋₆-alkyl, —N(C₁₋₆-alkyl)₂, —OH, halo, and C(O)R^{10d};
R^{10d} is selected from the group consisting of H, —OH, C₁₋₆-alkyl and —OC₁₋₆-alkyl; and
*1 indicates the point of attachment to A¹ and *9 indicates the point of attachment to A⁹.

In further embodiments, A¹⁰ is

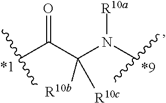

wherein
R^{10a} is H or CH₃;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents selected from —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1 or 2 substituents selected from C₁₋₃-alkyl and —OH; and
R^{10d} is selected from the group consisting of H, —OH, C₁₋₆-alkyl and —OC₁₋₆-alkyl.

In further embodiments, A¹⁰ is

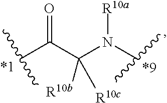

wherein
R^{10a} is H or CH₃;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents selected from —C(O)OH, —OH, —NH₂, —NHC₁₋₆-alkyl, —N(C₁₋₆-alkyl)₂, and —NH—C(O)R^{10d}; or A¹⁰ is a moiety of formula (IIA):

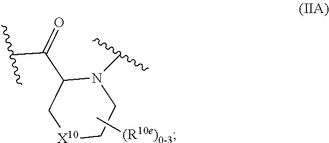

wherein:
X¹⁰ is absent or selected from the group consisting of O, NH, or CHR^{10e}; and
each R^{10e} is independently selected from the group consisting of C₁₋₃-alkyl, —NH₂, —NHC₁₋₆-alkyl, —N(C₁₋₆-alkyl)₂, —OH, halo, and C(O)R^{10d}, wherein R^{10d} is selected from the group consisting of H, —OH, and C₁₋₆-alkyl and —OC₁₋₆-alkyl.

In further embodiments, A¹⁰ is

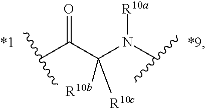

wherein
R^{10a} is H or CH₃;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with a substituent selected from —C(O)OH, —OH, —NH₂, —NHC₁₋₆-alkyl, —N(C₁₋₆-alkyl)₂, and —NH—C(O)R^{10d}; and
R^{10d} is selected from the group consisting of H, —OH, C₁₋₆-alkyl and —OC₁₋₆-alkyl.

In further embodiments, A¹⁰ is

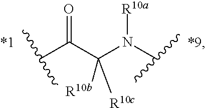

wherein
R^{10a} is H or CH₃;
R^{10b} is selected from the group consisting of H and C₁₋₃-alkyl;
R^{10c} is selected from the group consisting of H and C₁₋₆-alkyl.

In further embodiments, A¹⁰ is a moiety of formula (IIA):

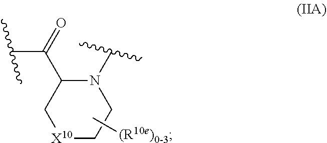

wherein:
X¹⁰ is absent or selected from the group consisting of O, N, or CHR^{10e}; and
   each R^{10e} is independently selected from —OH, —NH₂, —C₁₋₃-alkyl, halo, and —C(O)R^{10d}, wherein R^{10d} is selected from the group consisting of H, —OH, and C₁₋₆-alkyl and —OC₁₋₆-alkyl.

In further embodiments, A¹⁰ is a moiety of formula (IIA):

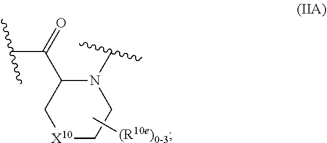

wherein:
X¹⁰ is absent or selected from the group consisting of O and CHR^{10e}; and
each R^{10e} is independently selected from —OH, and —C₁₋₆-alkyl.

In further embodiments, A¹⁰ is a moiety of formula (IIA):

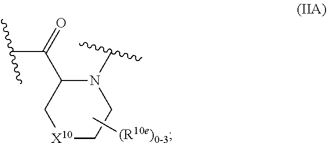

wherein:
X¹⁰ is absent or selected from the group consisting of O and CHR^{10e}; and
each R^{10e} is independently selected from —OH, and —CH₃.

In further embodiments, A¹⁰ is selected from the group consisting of:

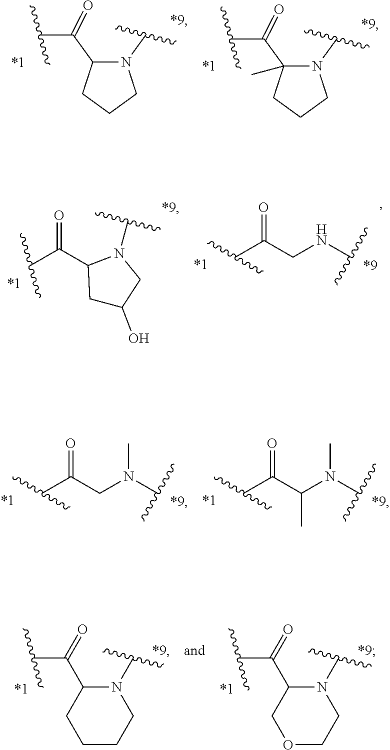

wherein *1 indicates the point of attachment to A¹ and *9 indicates the point of attachment to A⁹.

In further embodiments, A¹⁰ is selected from the group consisting of:

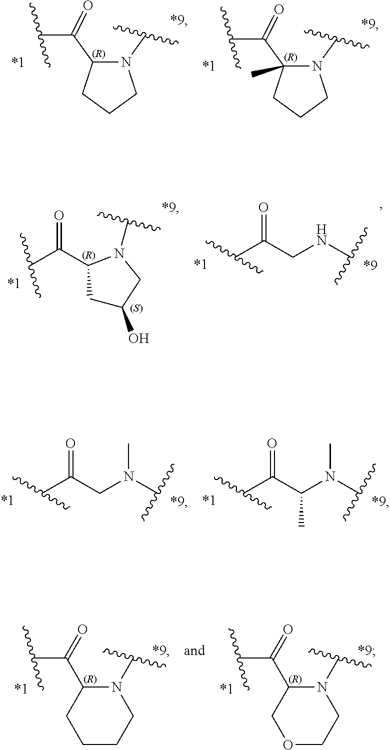

wherein *1 indicates the point of attachment to A¹ and *9 indicates the point of attachment to A⁹.

In further embodiments, A¹⁰ is

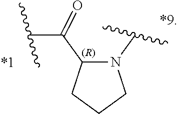

In further embodiments, A¹⁰ is

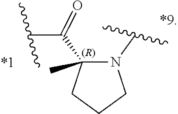

In further embodiments, A¹⁰ is

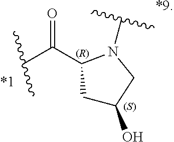

In further embodiments, A¹⁰ is

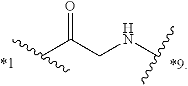

In further embodiments, A¹⁰ is

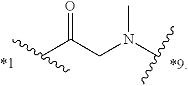

In further embodiments, A¹⁰ is

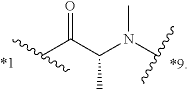

In further embodiments, A¹⁰ is

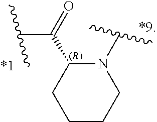

In further embodiments, A¹⁰ is

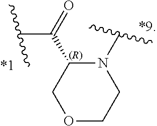

In some embodiments of the cyclic peptide (P) or compounds comprising cyclic peptide (P):
A¹ is a moiety of formula (II):

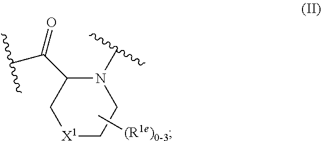

wherein:
X¹ is absent or selected from the group consisting of O, NH, CH₂, and CHR^{1e}; and
each R^{1e} is independently selected from the group consisting of C₁₋₃-alkyl, —NH₂, —NHC₁₋₆-alkyl, —N(C₁₋₆-alkyl)₂, —OH, halo, and C(O)R^{1d}, wherein R^{1d} is selected from the group consisting of H and —C₁₋₆-alkyl; or
A¹⁰ is a moiety of formula (IIA):

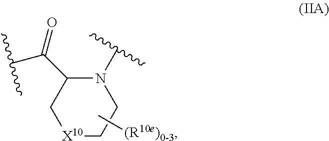

wherein:
X¹⁰ is absent or selected from the group consisting of O, NH, CH₂, and CHR^{10e}; and
each R^{10e} is independently selected from the group consisting of C₁₋₃-alkyl, —NH₂, —NHC₁₋₆-alkyl, —N(C₁₋₆-alkyl)₂, —OH, halo, and C(O)R^{10d}, wherein R^{10d} is selected from the group consisting of H, —OH, C₁₋₆-alkyl and —OC₁₋₆-alkyl;
provided that when A¹ is a moiety of formula (II), A¹⁰ is not a moiety of formula (IIA) and when A¹⁰ is a moiety of formula (IIA), A¹ is not a moiety of formula (II).

In some embodiments of the cyclic peptide (P) or compounds comprising cyclic peptide (P):
A¹ is a moiety of formula (II):

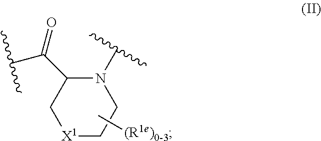

wherein:
X¹ is absent or selected from the group consisting of O, NH, CH₂, and CHR^{1e}; and
each R^{1e} is independently selected from the group consisting of C₁₋₃-alkyl, —NH₂, —NHC₁₋₆-alkyl, —N(C₁₋₆-alkyl)₂, —OH, halo, and C(O)R^{1d}, wherein R^{1d} is selected from the group consisting of H and —C₁₋₆-alkyl; and
A¹⁰ is a moiety of formula (IIA):

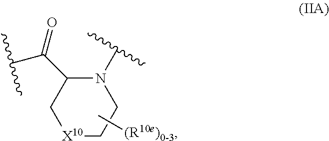

wherein:
X¹⁰ is absent or selected from the group consisting of O, NH, CH₂, and CHR^{10e}; and
each R^{10e} is independently selected from the group consisting of C₁₋₃-alkyl, —NH₂, —NHC₁₋₆-alkyl, —N(C₁₋₆-alkyl)₂, —OH, halo, and C(O)R^{10d}, wherein R^{10d} is selected from the group consisting of H, —OH, C₁₋₆-alkyl and —OC₁₋₆-alkyl.

In some embodiments of the cyclic peptide (P) or compounds comprising cyclic peptide (P):
(1) A¹ is a moiety of formula (II):

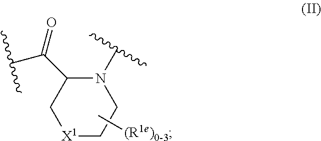

wherein:
X¹ is absent or selected from the group consisting of O, NH, CH₂, and CHR^{1e}; and each R^{1e} is independently selected from the group consisting of C₁₋₃-alkyl, —NH₂, —NHC₁₋₆-alkyl, —N(C₁₋₆-alkyl)₂, —OH, halo, and C(O)R^{1d}, wherein R^{1d} is H or —C₁₋₆-alkyl; or
A¹⁰ is a moiety of formula (IIA):

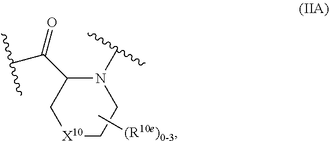

wherein:
   X¹⁰ is absent or selected from the group consisting of O, NH, and CHR^{10e}; and
each R^{10e} is independently selected from the group consisting of C₁₋₃-alkyl, —NH₂, —NHC₁₋₆-alkyl, —N(C₁₋₆-alkyl)₂, —OH, halo, and C(O)R^{10d}, wherein R^{10d} is selected from the group consisting of H, —OH, C₁₋₆-alkyl and —OC₁₋₆-alkyl;
provided that when A¹ is a moiety of formula (II), A¹⁰ is not a moiety of formula (IIA) and when A¹⁰ is a moiety of formula (IIA), A¹ is not a moiety of formula (II)
(2) A⁴ is a moiety of formula (III):

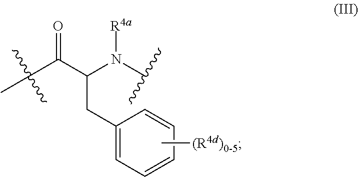

wherein:
R^{4a} is selected from the group consisting of H and C₁₋₃-alkyl,
each R^{4d} is independently selected from the group consisting of —C₁₋₆-alkyl, —OH, —OC₁₋₆-alkyl, —NH₂, halo, —NHC(O)R^{4f}, —C(O)NHR^{4f}, and —C(O)OR^{4f}, wherein the C₁₋₆-alkyl and —OC₁₋₆-alkyl of R^{4d} are each optionally substituted with 1, 2, or 3 substituents independently selected from the group consisting of —CN, —NH₂, —NHC₁₋₆-alkyl, —N(C₁₋₆-alkyl)₂, N₃, —OH, —C(O)OH, —C(O)NH₂, and 5-6 membered heteroaryl optionally substituted with —C₁₋₃-alkyl-NH₂ or —C₁₋₃-alkyl-OH; and
each R^{4f} is independently selected from the group consisting of H and C₁₋₆-alkyl; and
(3) A⁸ is a moiety of formula (IV):

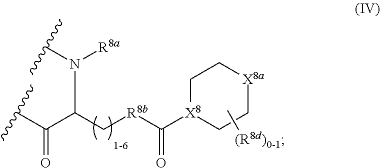

wherein:
X⁸ is selected from the group consisting of N and CH;
X^{8a} is selected from the group consisting of NH, NR^{8d}, O, CH₂, and CHR^{8d};
X^{8b} is selected from the group consisting of NH, NR^{8a}, O, and CH₂;
R^{8a} is selected from the group consisting of H and C₁₋₃-alkyl; and
R^{8d} is selected from the group consisting of —C₁₋₆-alkylC(O)OH and —C(O)OH;
wherein at least one of X⁸ and X^{8a} is N.

For example, in some embodiments of the cyclic peptide (P) or compounds comprising cyclic peptide (P):
A¹ is a moiety of formula (II), A⁴ is a moiety of formula (III), A⁸ is a moiety of formula (IV), and A¹⁰ is

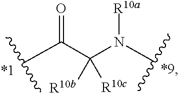

wherein
R^{10a} is H or CH₃;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents selected from —C(O)OH, —OH, —NH₂, —NHC₁₋₆-alkyl, —N(C₁₋₆-alkyl)₂, and —NH—C(O)R^{1c}.

In other embodiments of the cyclic peptide (P) or compounds comprising cyclic peptide (P):
A¹ is

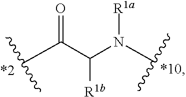

wherein
R^{1a} is H or CH₃;
R^{1b} is selected from the group consisting of H and C₁₋₆-alkyl, wherein the C₁₋₆-alkyl of R^{1b} is optionally substituted with a substituent selected from —C(O)OH, —OH, —NH₂, —NHC₁₋₆-alkyl, —N(C₁₋₆-alkyl)₂, and —NH—C(O)R^{1c};
R^{1c} is a 4-7 membered heterocycloalkyl, wherein the 4-7 membered heterocycloalkyl of R^{1c} is optionally substituted with a substituent selected from the group consisting of —C₁₋₆-alkyl-NH₂ and —C₁₋₆-alkyl-C(O)OH;
A⁴ is a moiety of formula (III);
A⁸ is a moiety of formula (IV); and
A¹⁰ is a moiety of formula (IIA).

In some embodiments of the cyclic peptide (P) or compounds comprising cyclic peptide (P):
when A¹ is a moiety of formula (II), A⁴ is a moiety of formula (III), and A⁸ is a moiety of formula (IV); then:
A¹ is selected from:

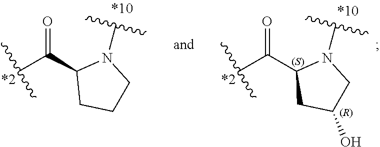

A⁴ is selected from:

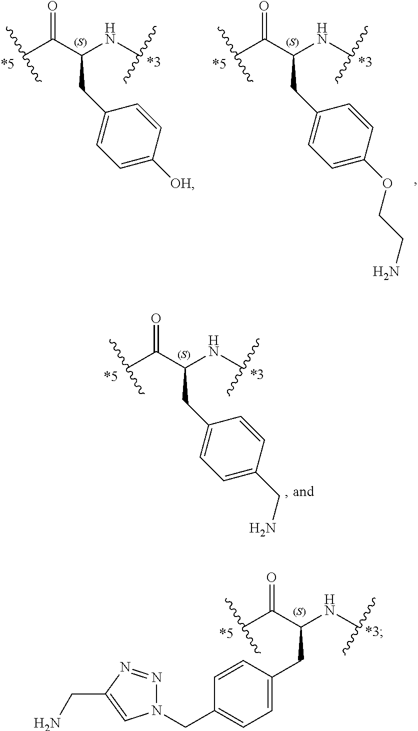

and
A⁸ is selected from:

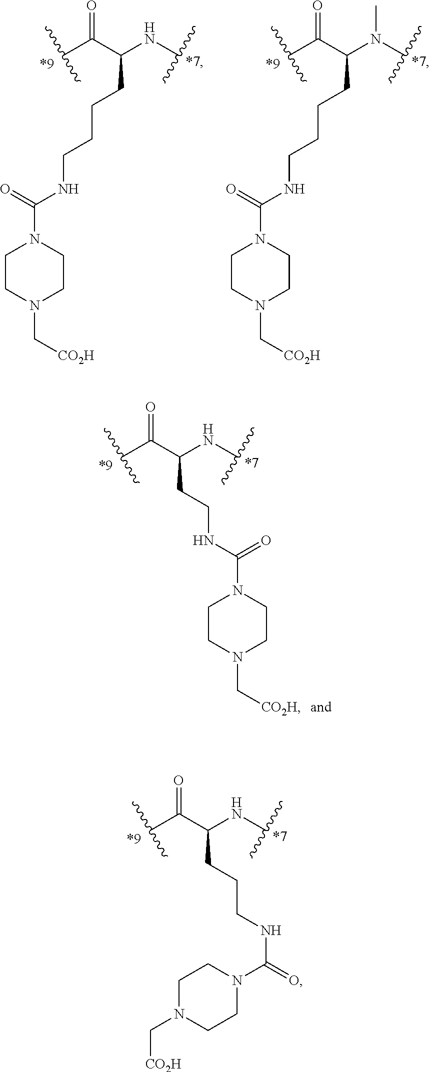 [SMILES: *N[C@@H](CCCCNC(=O)N1CCN(CC(=O)O)CC1)C(*)=O ; *C(=O)[C@H](CCCCNC(=O)N1CCN(CC(=O)O)CC1)N(*)C ; *N[C@@H](CCNC(=O)N1CCN(CC(=O)O)CC1)C(*)=O ; *N[C@@H](CCCNC(=O)N1CCN(CC(=O)O)CC1)C(*)=O]

In some embodiments of the cyclic peptide (P) or compounds comprising cyclic peptide (P):
when A⁴ is a moiety of formula (III), A⁸ is a moiety of formula (IV), and A¹⁰ is a moiety of formula (IIA); then:
A⁴ is selected from:

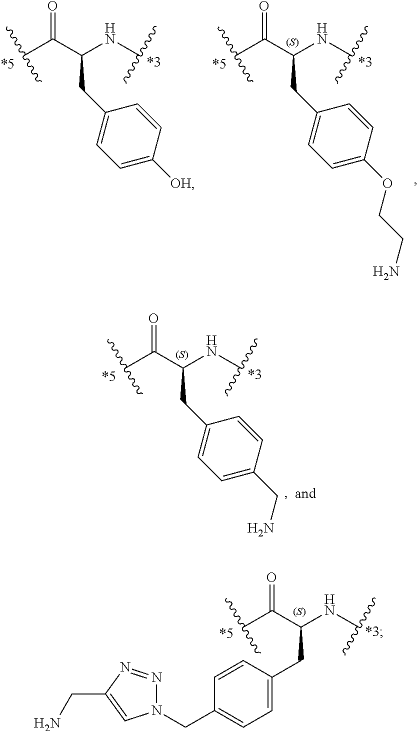

A⁴ is selected from:

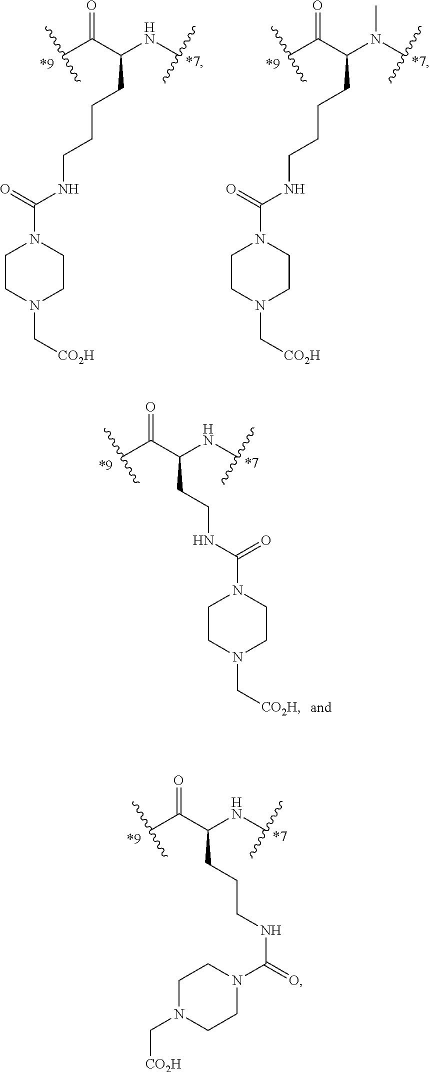 [SMILES: *N[C@@H](CCCCNC(=O)N1CCN(CC(=O)O)CC1)C(*)=O ; *C(=O)[C@H](CCCCNC(=O)N1CCN(CC(=O)O)CC1)N(*)C ; *N[C@@H](CCNC(=O)N1CCN(CC(=O)O)CC1)C(*)=O ; *N[C@@H](CCCNC(=O)N1CCN(CC(=O)O)CC1)C(*)=O]

and
A¹⁰ is selected from:

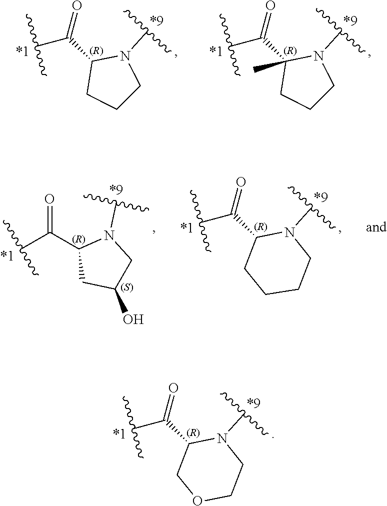

In some embodiments, the cyclic peptide (P) or compounds comprising cyclic peptide (P) comprise the following structure:

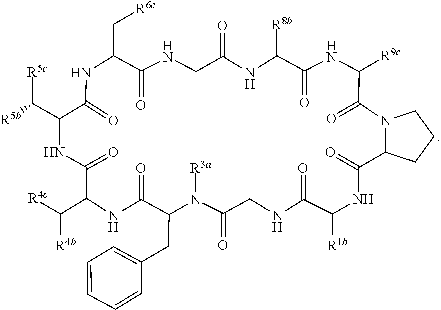

In further embodiments, the cyclic peptide (P) or compounds comprising cyclic peptide (P) comprise the following structure:

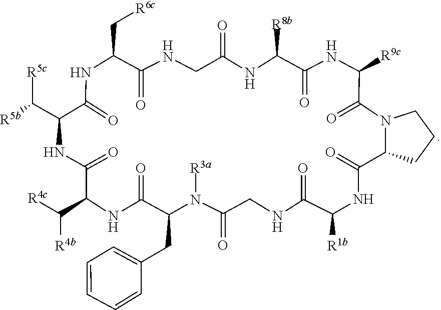

In still further embodiments, the cyclic peptide (P) or compounds comprising cyclic peptide (P) comprise the following structure:

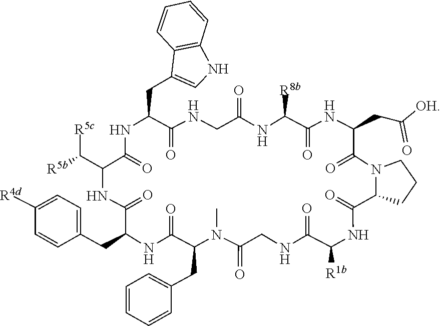

In some embodiments, the cyclic peptide (P) or compounds comprising cyclic peptide (P) comprise the following structure:

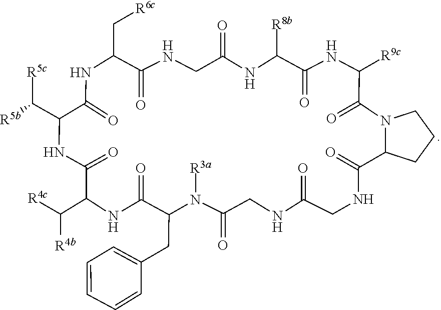

In further embodiments, the cyclic peptide (P) or compounds comprising cyclic peptide (P) comprise the following structure:

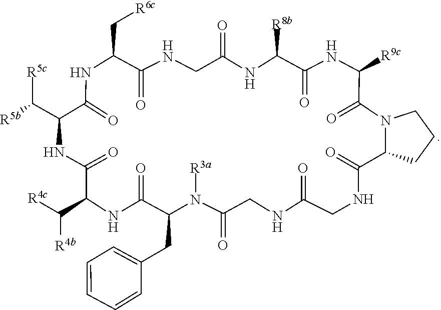

In still further embodiments, the cyclic peptide (P) or compounds comprising cyclic peptide (P) comprise the following structure:

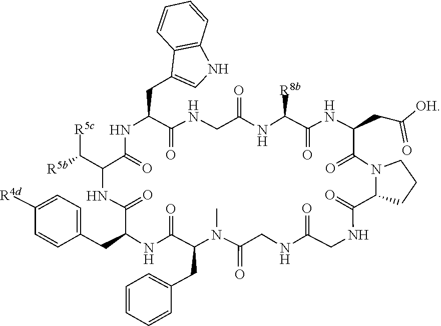

In some embodiments, the cyclic peptide (P) or compounds comprising cyclic peptide (P) comprise the following structure:

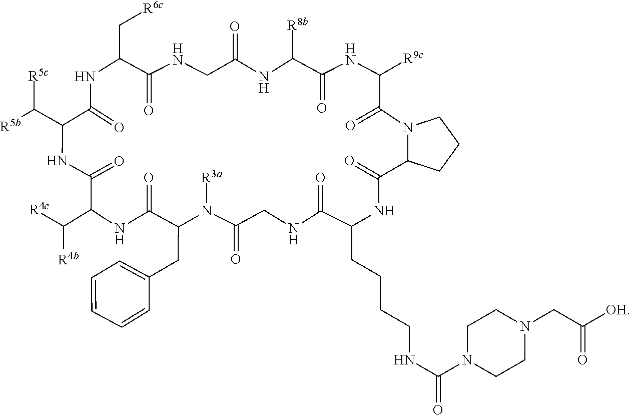

In further embodiments, the cyclic peptide (P) or compounds comprising cyclic peptide (P) comprise the following structure:

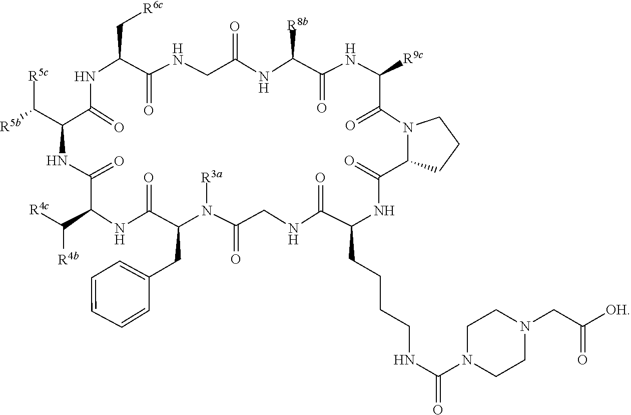

In still further embodiments, the cyclic peptide (P) or compounds comprising cyclic peptide (P) comprise the following structure:

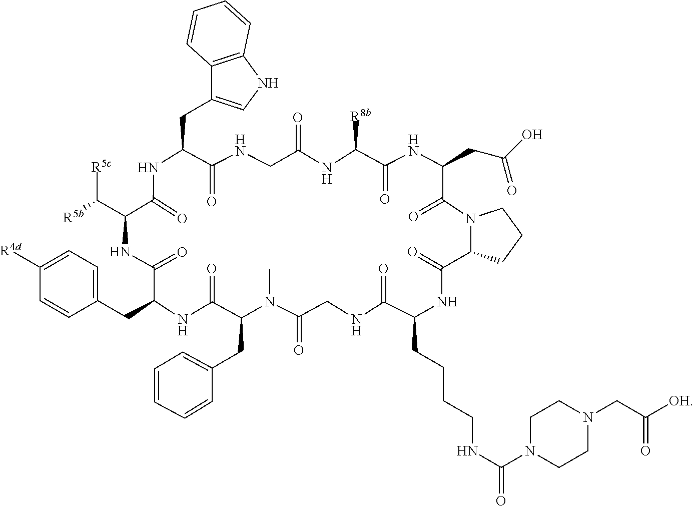

In certain embodiments, the cyclic peptide (P) is of Formula (P-I): *^{α}-A¹-Gly-A³-A⁴-A⁵-A⁶-Gly-A⁸-Asp-(D-Pro)-*^{β}, (SEQ ID NO: 192) wherein
*^{α} indicates the point of attachment of A¹ to D-Pro;
*^{β} indicates the point of attachment of D-Pro to A¹, wherein A¹ and D-Pro are attached so Formula (P-I) is a cyclized (cyclic) peptide (P);

A¹ is any amino acid (e.g., Asp, Gly, NMeGly, Glu, Lys, Lys(Cy5), K(COpipzaa), Ser, D-Ser, hSer, Pro, trans4Hyp; for example, Gly);
A³ is: Phe or NMePhe;
A⁴ is Phe;
and A⁴ is bound to linker L¹, wherein L¹ is bound to M, and M is an imaging agent, chelating agent, radionuclide, or cytotoxic drug (e.g., M is a chelator optionally radiolabeled with a radionuclide);
A⁵ is: Ahp, Aoc, hS(Pr), or Q(Pyrro(2CN4F2));
A⁶ is: Trp, Trp(Boc), W(6OH), W(7Cl), W(7F), W7N, or W(7OMe); and
A⁸ is: any amino acid (e.g., Tyr, K(COpipzaa)).

In certain further embodiments, the cyclic peptide (P) of Formula (P-I) is of Formula (P-I-A): *^{α}-Gly-Gly-A³-A⁴-A⁵-A⁶-Gly-A⁸-Asp-(D-Pro)-*^{β}-, (SEQ ID NO: 193) wherein
*^{α} indicates the point of attachment of A¹ to D-Pro;
*^{β} indicates the point of attachment of D-Pro to A¹, wherein A¹ and D-Pro are attached so Formula (P-I-A) is a cyclized peptide (P);
A³ is: Phe or NMePhe;
A⁴ is Phe;
and A⁴ is bound to linker L¹, wherein L¹ is bound to M, and M is a chelator (e.g., a chelator of Tables 3-7) optionally radiolabeled with a radionuclide (e.g., ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th);
A⁵ is: Ahp, Aoc, hS(Pr), or Q(Pyrro(2CN4F2));
A⁶ is: Trp, Trp(Boc), W(6OH), W(7Cl), W(7F), W7N, or W(7OMe); and
A⁸ is: any amino acid (e.g., Tyr, K(COpipzaa)).

In certain further embodiments, the cyclic peptide (P) is of Formula (P-I-A-i): *^{α}-Gly-Gly-A³-A⁴-A⁵-A⁶-Gly-A⁸-Asp-(D-Pro)-*^{β}, (SEQ ID NO: 193) wherein
*^{α} indicates the point of attachment of A¹ to D-Pro;
*^{β} indicates the point of attachment of D-Pro to A¹, wherein A¹ and D-Pro are attached so Formula (P-I-A-i) is a cyclized peptide (P);
where *3 indicates the point of attachment to A³ and *5 indicates the point of attachment to A⁵;
A³ is: Phe or NMePhe;
A⁴ is Phe;
and A⁴ is bound to linker L¹, wherein L¹ is bound to M, and M is a chelator (e.g., a chelator of Tables 3-7, for example, a chelator of Tables 3-5) optionally radiolabeled with a radionuclide (e.g., ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th);
wherein A⁴ and L¹ together have a structure selected from: F(4amPEG2NH2), F(4amPEG2NH), F(40EtNH2), F(4am), F(4amGly), F(4amMeGly), F(4amMeBAla), F(4amBAla), F4(MeTriazolylMeNH2), F(4CCH), F(4amPEG2NH-p-SCN)—, F(4amPEG2NHCOPy3(4F)), F(4amPEG2NHCOPy3(4(NMe3))), F((4amPEG2NH)2-bE-NH, or F((40EtNHPEG3NH)2-bE-NH (for example, (e.g.,

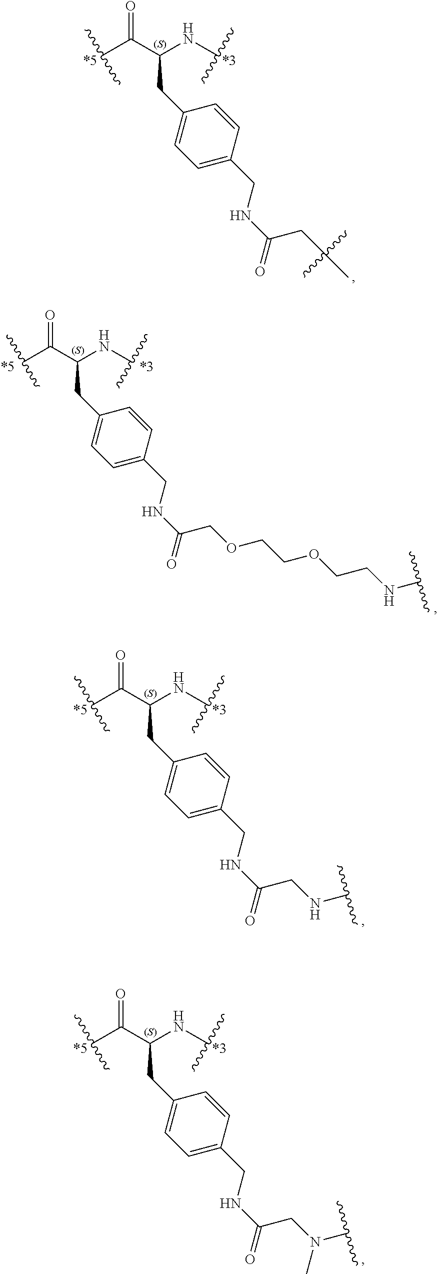

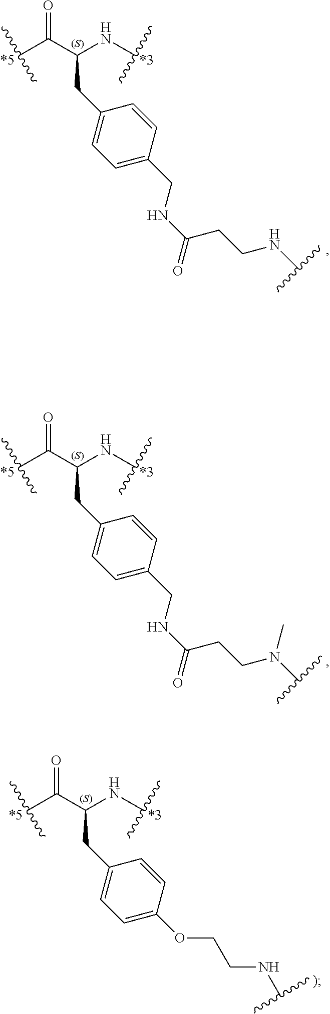

A⁵ is: Ahp, Aoc, hS(Pr), or Q(Pyrro(2CN4F2));
A⁶ is: Trp, Trp(Boc), W(6OH), W(7Cl), W(7F), W7N, or W(7OMe); and
A⁸ is: any amino acid (e.g., Tyr, K(COpipzaa)).

In certain embodiments, the cyclic peptide (P) is a cyclic peptide selected from any one of Examples A1 to A76, or a pharmaceutically acceptable salt, solvate, stereoisomer, or tautomer thereof.

In certain embodiments, the cyclic peptide (P) is a cyclic peptide selected from any one of Examples A1 to A74, or a pharmaceutically acceptable salt, solvate, stereoisomer, or tautomer thereof. In certain embodiments, a compound of the present disclosure (e.g., ligand, for example, radioligand) is a compound of the table of Example III, or a pharmaceutically acceptable salt, solvate, stereoisomer, or tautomer thereof.

### Albumin Binders

The FAP-targeting cyclic peptide (P), or the compound comprising a cyclic peptide (P), of the present disclosure may further comprise an albumin binder. For example, an albumin binder may be attached to the cyclic peptide via a side chain of any one of A¹-A¹⁰. In one or more embodiments, an albumin binder is connected to the cyclic peptide via a side chain of A².

Any compound, peptide, polypeptide, or other moiety known in the art as being capable of binding albumin, i.e., having albumin binding affinity, may be connected to the cyclic peptide (or any of the compounds comprising said cyclic peptide) of the instant disclosure as an albumin binder. Addition of an albumin binder to the cyclic peptide may prolong the bioavailability of the disclosed FAP-targeting compounds, thereby enhancing tumor uptake. Exemplary albumin binders are described in Zorzi et al. Non-covalent albumin-binding ligands for extending the circulating half-life of small biotherapeutics. Med. Chem. Comm. 2019 Jun. 6; 10(7):1068-1081, Brandt et al. Mini-review: Targeted radiopharmaceuticals incorporating reversible, low molecular weight albumin binders. Nucl. Med. Biol. 2019 March; 70:46-52, and Lau et al. Bench to Bedside: Albumin Binders for Improved Cancer Radioligand Therapies. Bioconjug. Chem. 2019 Mar. 20; 30(3):487-502. 10.1021/acs.bioconjchem.8b00919. Epub 2019 Jan. 23. PMID: 30616340, which are each incorporated herein by reference in their entireties.

In one or more embodiments, the cyclic peptide, or any of the compounds disclosed herein comprising said cyclic peptide, is substituted with an albumin binder selected from fatty acids and derivatives thereof. Such fatty acids and derivatives thereof include, e.g.,

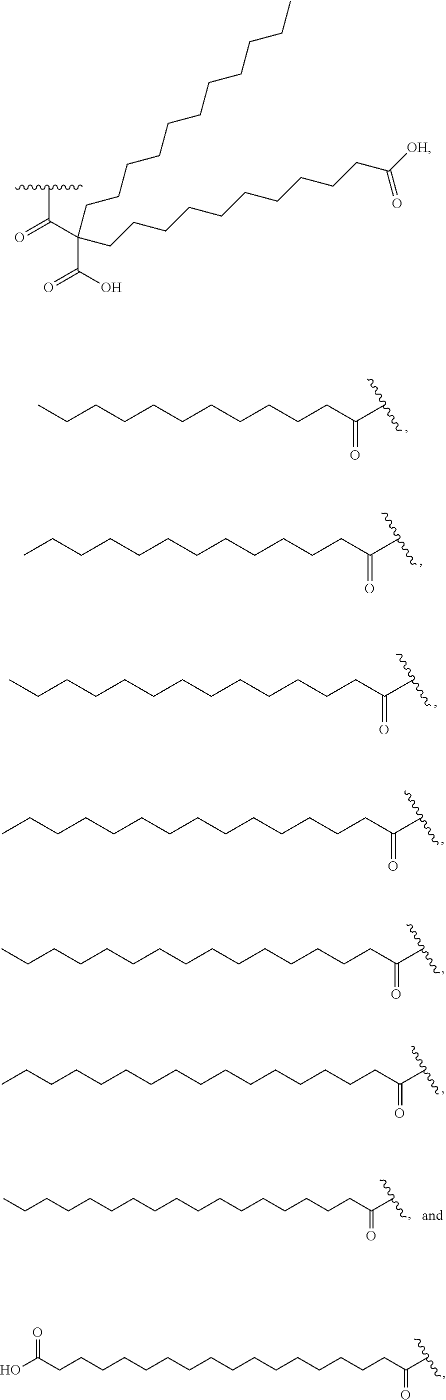

In one or more embodiments, the cyclic peptide, or any of the compounds disclosed herein comprising said cyclic peptide, is substituted with an albumin binder selected from:

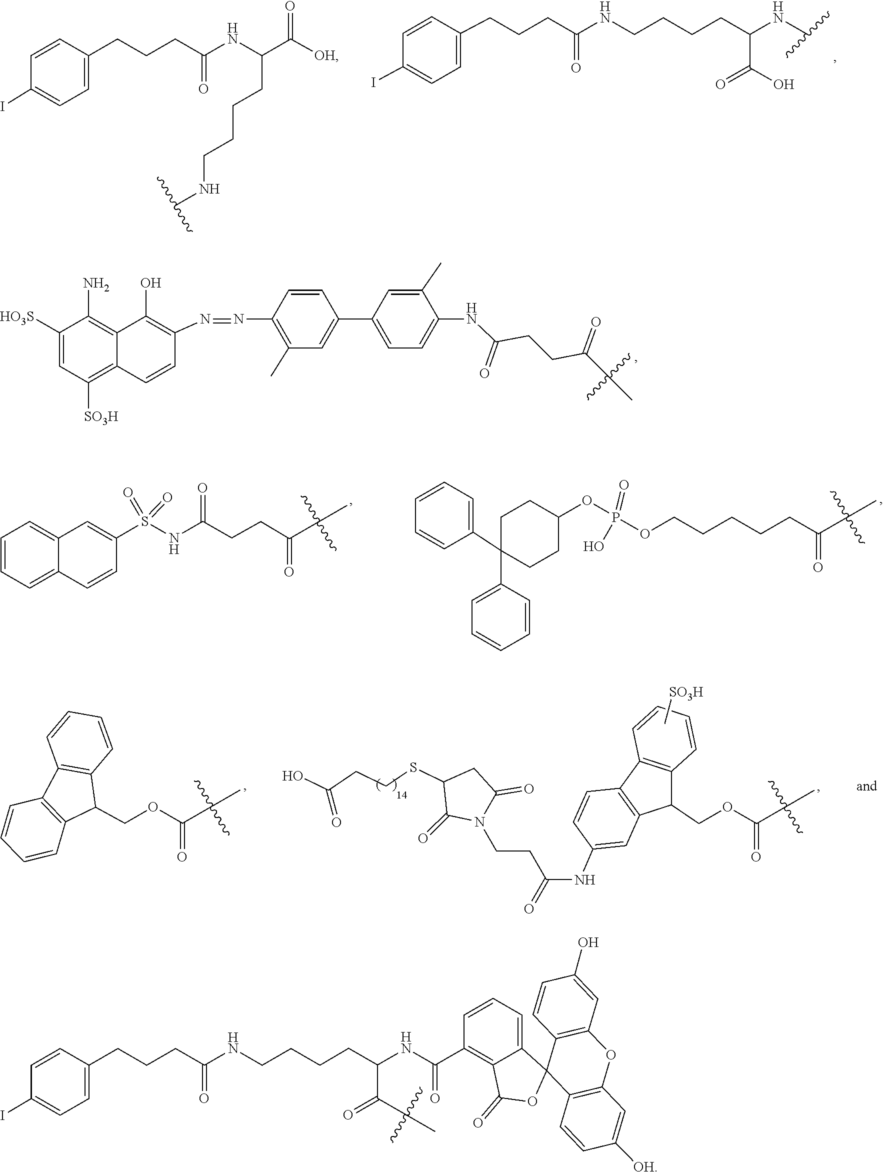

In various embodiments, the albumin binder is 4-(p-iodophenyl)butyric acid or a derivative thereof, such as, for example:

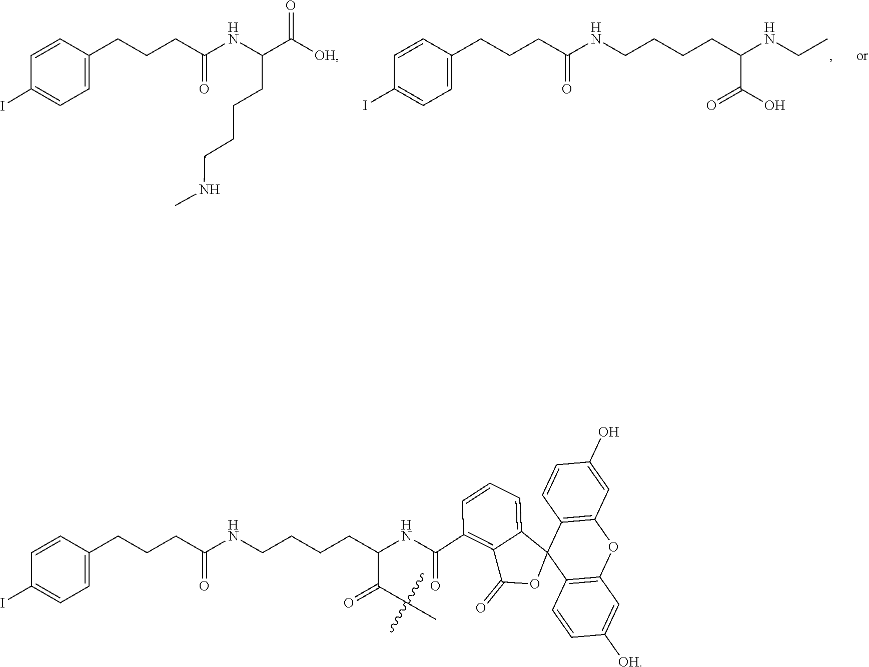 [SMILES: CNCCCCC(NC(=O)CCCc1ccc(I)cc1)C(=O)O ; CCNC(CCCCNC(=O)CCCc1ccc(I)cc1)C(=O)O ; *C(=O)C(CCCCNC(=O)CCCc1ccc(I)cc1)NC(=O)c1cccc2c1C(=O)OC21c2ccc(O)cc2Oc2cc(O)ccc21]

In some embodiments, the albumin binder is:

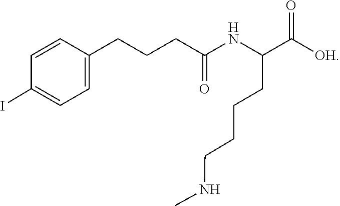 [SMILES: CNCCCCC(NC(=O)CCCc1ccc(I)cc1)C(=O)O]

In some embodiments, the albumin binder is:

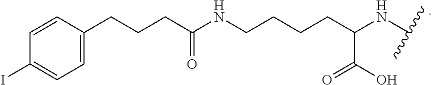 [SMILES: *NC(CCCCNC(=O)CCCc1ccc(I)cc1)C(=O)O]

In some embodiments, the albumin binder is Evans blue, or a derivative thereof. In some embodiments, the albumin binder is:

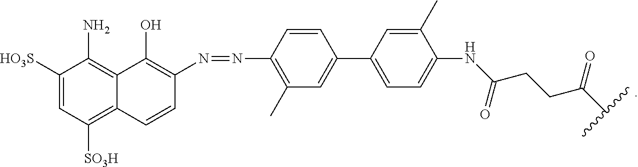 [SMILES: *C(=O)CCC(=O)Nc1ccc(-c2ccc(N=Nc3ccc4c(S(=O)(=O)O)cc(S(=O)(=O)O)c(N)c4c3O)c(C)c2)cc1C]

In an embodiment, an albumin binder is connected to the cyclic peptide via a side chain of A², and the structure of A² and the albumin binder together have the structure:

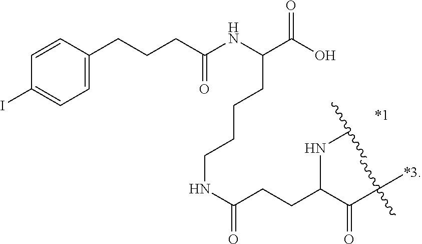

In an embodiment, an albumin binder is connected to the cyclic peptide (or compound comprising the cyclic peptide) via a side chain of A², and the structure of A² and the albumin binder together have the structure:

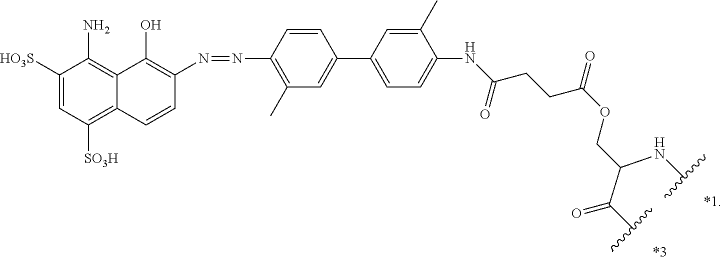

In some embodiments when the cyclic peptide, or compound comprising said cyclic peptide, is substituted with an albumin binder cyclic peptide, said cyclic peptide, is a compound selected from Examples F1-3, F1, F2-1, and F2, or a pharmaceutically acceptable salt, solvate, stereoisomer, or tautomer thereof.

### L′ (e.g., of Compounds of Formulae (I), (Ia), (Ib), (Ic), and (Id))

The compounds disclosed herein (e.g., compounds of Formulae (I), (Ia), (Ib), (Ic), and (Id) comprise L¹ between P and M. In some embodiments, L¹ is a bond (i.e., a bond between P and M). In some embodiments, L¹ is a linker. As used herein, the term “linker” means a structural component that connects two parts of a compound. Linkers can comprise an atom such as oxygen or sulfur, a unit such as NR, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl. In any of these structural components, one or more methylenes can be interrupted or terminated by O, S, S(O), SO₂, N(R′), C(O), substituted or unsubstituted aryl, substituted or unsubstituted heteroaryl, substituted or unsubstituted heterocyclic; where R′ is hydrogen, acyl, aliphatic or substituted aliphatic. In some embodiments, the linker L is between one to about twenty-four atoms, preferably one to about twelve atoms, preferably between about one to about eight atoms, more preferably one to about six atoms, and most preferably about four to about six atoms.

The linker may be attached to any amino acid site of the cyclic peptide (P) to form a linkage between P and M. For example, the linker may be attached to the FAP-targeting compound via any one of A¹-A¹⁰. In some embodiments, the linker is attached to a side chain of any one of A¹-A¹⁰. In other embodiments, the linker is attached to a backbone nitrogen of any one of A-A¹⁰. In some embodiments, the linker is attached to the side chain or backbone nitrogen of A¹. In some embodiments, the linker is attached to the side chain or backbone nitrogen of A². In some embodiments, the linker is attached to the side chain or backbone nitrogen of A³. In some embodiments, the linker is attached to the side chain or backbone nitrogen of A⁴. In some embodiments, the linker is attached to the side chain or backbone nitrogen of A⁵. In some embodiments, the linker is attached to the side chain or backbone nitrogen of A⁶. In some embodiments, the linker is attached to the side chain or backbone nitrogen of A⁷. In some embodiments, the linker is attached to the side chain or backbone nitrogen of A⁸. In some embodiments, the linker is attached to the side chain or backbone nitrogen of A⁹. In some embodiments, the linker is attached to the side chain or backbone nitrogen of A¹⁰.

Analogously, the linker may be attached to any site capable of forming a covalent attachment on the chelator to form a linkage between P and M. For example, the linker may be attached to the chelator via a heteroatom, the functional group of a functionalized heteroatom, or an alkyl, cycloalkyl, or aryl group separating the heteroatoms of the chelator. In some embodiments, the linker is attached to a heteroatom of the chelator. In other embodiments, the linker is attached to the functional group of a functionalized heteroatom of the chelator. In yet other embodiments, the linker is attached to an alkyl, cycloalkyl, aryl, or heteroaryl group separating the heteroatoms of the chelator. In some embodiments, the linker is attached to an alkyl group separating the heteroatoms of the chelator. In some embodiments, the linker is attached to a cycloalkyl group separating the heteroatoms of the chelator. In some embodiments, the linker is attached to an aryl group separating the heteroatoms of the chelator.

In some embodiments, the linker is a covalent bond. Alternatively, in some embodiments, the linker is a divalent moiety other than a covalent bond. In embodiments in which the linker is a divalent moiety other than a bond, the linker comprises one or more functional groups capable of forming a covalent attachment with the cyclic peptide (P) or chelator (M). Suitable functional groups include, but are not limited to, amine, azide, amide, carboxylic acid, hydroxy, alkoxy, nitrile, alkene, and alkyne functional groups.

L¹ may be any linker known in the art. In some embodiments, the linker includes a group such as, for example, an amino acid, or derivative thereof, CH₂, CH₂CH₂, cycloalkylene, alkylene, arylene, alkylarylene, heteroarylene, heterocycloalkylene, (CR⁴R⁵)ₚO(CR⁴R⁵)_{q}, (CR⁴R⁵)ₚN(CR⁴R⁵)_{q}, (CR⁴R⁵)ₚS(CR⁴R⁵)_{q}, wherein cycloalkylene, alkylene, arylene, alkylarylene, heteroarylene, and heterocycloalkylene are optionally substituted with 1, 2, or 3 substituents independently selected from halo, C₁₋₆ alkyl, C₂₋₆ alkenyl, C₂₋₆ alkynyl, C₁₋₆ haloalkyl, halosulfanyl, CN, NO₂, N₃, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)NR^{c}R^{d}, NR^{c}C(O)OR^{a}, C(═NR^{g})NR^{c}R^{d}, NR^{c}C(═NR^{g})NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d},
wherein R⁴ and R⁵ are independently selected from H, halo, OH, C₁₋₆ alkyl, C₂₋₆ alkenyl, C₂₋₆ alkynyl, C₁₋₆ alkoxy, alkoxyalkyl, cyanoalkyl, heterocycloalkyl, cycloalkyl, C₁₋₆ haloalkyl, CN, and NO₂,
R^{a}, R^{b}, R^{c}, and R^{d} are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from —OH, —CN, amino, halo, C₁₋₆ alkyl, C₁₋₆alkoxy, C₁₋₆haloalkyl, and C₁₋₆haloalkoxy
R^{e}, R^{f}, and R^{g} are each independently selected from the group consisting of H and C₁₋₁₀ alkyl;
p is 0, 1, 2, 3, 4, 5, or 6; and
q is 0, 1, 2, 3, 4, 5, or 6.

In various embodiments, L¹ is a linker comprising the following structure:

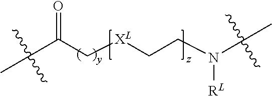

wherein:
X^{L} is CH₂, N, or O;
R^{L} is H, C₁₋₃-alkyl, or acyl;
y, independently, at each occurrence, is 0, 1, 2, 3, 4, 5, or 6; and
z, independently at each occurrence, is 0, 1, 2, 3, or 4.

In some embodiments, L¹ is a linker comprising the following structure:

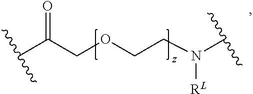

wherein:
R^{L} is selected from the group consisting of H and C₁₋₃-alkyl; and
z is 0, 1, 2, 3, or 4.

In some embodiments, L¹ is a linker of the following structure:

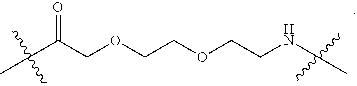 [SMILES: *NCCOCCOCC(*)=O]

In other embodiments, L¹ is a linker comprising the following structure:

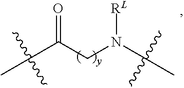

wherein:
R^{L} is selected from the group consisting of H or C₁₋₃-alkyl; and
y is 1, 2, 3, or 4.

In various embodiments, L¹ is a linker comprising the following structure:

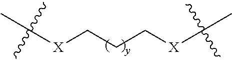

wherein:
X, independently at each occurrence, is CH₂, NR^{L} or O;
y, independently, at each occurrence, is 0, 1, 2, 3, 4, 5, or 6; and
R^{L}, independently at each occurrence, is H, C₁₋₃-alkyl, or acyl.

In some embodiments, L¹ is a linker comprising the following structure:

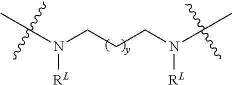

wherein:
R^{L} is independently at each occurrence selected from the group consisting of H, C₁₋₃-alkyl, and acyl; and
y is 0, 1, 2, 3, 4, 5, or 6.

In some embodiments, L¹ is a linker of the following structure:

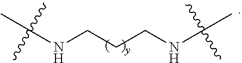

In some embodiments, X is CH₂. In some embodiments, X is N. In some embodiments, X is NR^{L}. In some embodiments, X is O.

In some embodiments, y is 0, 1, 2, 3, 4, 5, or 6. In some embodiments, y is 0, 1, 2, 3, 4, or 5. In some embodiments, y is 0, 1, 2, 3, or 4. In some embodiments, y is 0, 1, 2, or 3. In some embodiments, y is 0, 1, or 2. In some embodiments, y is 0 or 1. In some embodiments, y is 6. In some embodiments, y is 5. In some embodiments, y is 4. In some embodiments, y is 3. In some embodiments, y is 2. In some embodiments, y is 1. In some embodiments, y is 0.

In some embodiments, z is 0, 1, 2, 3, or 4. In some embodiments, z is 0, 1, 2, or 3. In some embodiments, z is 1, 2, 3, or 4. In some embodiments, z is 0, 1, or 2. In some embodiments, z is 1, 2, or 3. In some embodiments, z is 1 or 2. In some embodiments, z is 0 or 2. In some embodiments, z is 4. In some embodiments, z is 3. In some embodiments, z is 2. In some embodiments, z is 1. In some embodiments, z is 0.

In some embodiments, R^{L} is H, C₁₋₃-alkyl, or acyl. In some embodiments, R^{L} is H or C₁₋₃-alkyl. In some embodiments, R^{L} is H or acyl. In some embodiments, R^{L} is C₁₋₃-alkyl or acyl. In some embodiments, R^{L} is H. In some embodiments, R^{L} is C₁₋₃-alkyl. In some embodiments, R^{L} is acyl.

In some embodiments, L¹ is a linker comprising a structure selected from the group consisting

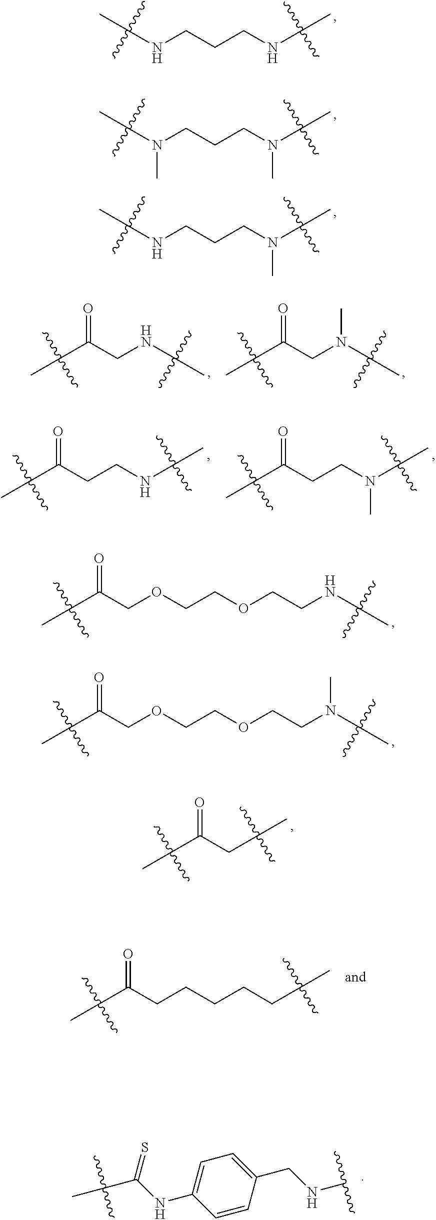

In various embodiments, L¹ is a linker comprising the following structure:

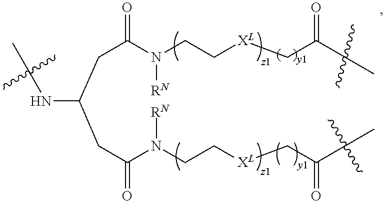

wherein:
X^{L} is CH₂, N, or O;
R^{N} is H, C₁₋₃-alkyl, or acyl;
y1, independently, at each occurrence, is 0, 1, 2, 3, 4, 5, or 6; and
z1, independently at each occurrence, is 0, 1, 2, 3, or 4.

In some embodiments, L¹ is a linker comprising the following structure:

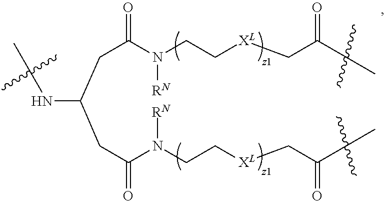

wherein:
R^{N} is selected from the group consisting of H and C₁₋₃-alkyl; and
z1 is 0, 1, 2, 3, or 4.

In some embodiments, L¹ is a linker of the following structure:

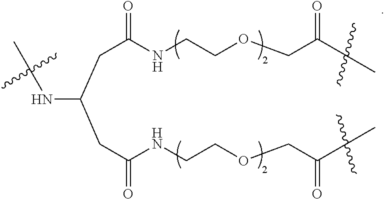

In some embodiments, L¹ is a linker of the following structure:

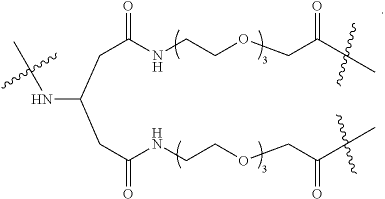

In some embodiments, X^{L} is CH₂. In some embodiments, X is N. In some embodiments, X is NR^{L}. In some embodiments, X^{L} is O.

In some embodiments, y1 is 0, 1, 2, 3, 4, 5, or 6. In some embodiments, y1 is 0, 1, 2, 3, 4, or 5. In some embodiments, y1 is 0, 1, 2, 3, or 4. In some embodiments, y1 is 0, 1, 2, or 3. In some embodiments, y1 is 0, 1, or 2. In some embodiments, y1 is 0 or 1. In some embodiments, y1 is 6. In some embodiments, y1 is 5. In some embodiments, y1 is 4. In some embodiments, y1 is 3. In some embodiments, y1 is 2. In some embodiments, y1 is 1. In some embodiments, y1 is 0. In some embodiments, z1 is 0, 1, 2, 3, or 4. In some embodiments, z1 is 0, 1, 2, or 3. In some embodiments, z1 is 1, 2, 3, or 4. In some embodiments, z1 is 0, 1, or 2. In some embodiments, z1 is 1, 2, or 3. In some embodiments, z1 is 1 or 2. In some embodiments, z1 is 0 or 2. In some embodiments, z1 is 4. In some embodiments, z1 is 3. In some embodiments, z1 is 2. In some embodiments, z1 is 1. In some embodiments, z1 is 0.

In some embodiments, R^{N} is H, C₁₋₃-alkyl, or acyl. In some embodiments, R^{N} is H or C₁₋₃-alkyl. In some embodiments, R^{N} is H or acyl. In some embodiments, R^{N} is C₁₋₃-alkyl or acyl. In some embodiments, R^{N} is H. In some embodiments, R^{N} is C₁₋₃-alkyl. In some embodiments, R^{N} is acyl.

Alternatively, in some embodiments, L¹ comprises a peptidic linker having the formula:

-(A¹¹)ₘ-,
wherein A¹¹ is an amino acid or a derivative thereof; and
m is an integer from 1 to 10.

In some embodiments, each A¹¹ is a natural amino acid. In some embodiments, each A¹¹ is a non-natural amino acid. In some embodiments, A¹¹ is, independently at each occurrence, selected from a natural amino acid and a non-natural amino acid. In some embodiments, each A¹¹ is an L amino acid. In some embodiments, A¹¹ is a D amino acid. In some embodiments, A¹¹ is, independently at each occurrence, selected from an L amino acid and a D amino acid.

In some embodiments, A¹¹ is, independently at each occurrence, selected from Asp, Glu, Gln, Gly, Lys, Phe, 5-aminovaleric acid, 8-amino-3,6-dioxaoctanoic acid, and derivatives thereof. In some embodiments, A¹¹ is, independently at each occurrence, selected from Asp, Glu, Gln, Gly, Lys, Phe, 5-aminovaleric acid, and 8-amino-3,6-dioxaoctanoic acid. In some embodiments, A¹¹ is, independently at each occurrence, selected from a derivative of Asp, a derivative of Glu, a derivative of Gln, a derivative of Gly, a derivative of Lys, a derivative of Phe, a derivative of 5-aminovaleric acid, and a derivative of 8-amino-3,6-dioxaoctanoic acid. In some embodiments, each A¹¹ is Asp. In some embodiments, each A¹¹ is Glu. In some embodiments, each A¹¹ is Gln. In some embodiments, each A¹¹ is Gly. In some embodiments, each A¹¹ is Lys. In some embodiments, each A¹¹ is Phe. In some embodiments, each A¹¹ is 5-aminovaleric acid. In some embodiments, each A¹¹ is 8-amino-3,6-dioxaoctanoic acid. In some embodiments, each A¹¹ is a derivative of Asp. In some embodiments, each A¹¹ is a derivative of Glu. In some embodiments, each A¹¹ is a derivative of Gln. In some embodiments, each A¹¹ is a derivative of Gly. In some embodiments, each A¹¹ is a derivative of Lys. In some embodiments, each A¹¹ is a derivative of Phe. In some embodiments, each A¹¹ is a derivative of 5-aminovaleric acid. In some embodiments, each A¹¹ is a derivative of 8-amino-3,6-dioxaoctanoic acid.

In some embodiments, A¹¹ is, independently at each occurrence, selected from Glu, Gln, Gly, Lys, and derivatives thereof. In some embodiments, A¹¹ is, independently at each occurrence, selected from Glu, Gln, Gly, and Lys. In some embodiments, A¹¹ is, independently at each occurrence, selected from a derivative of Glu, a derivative of Gln, a derivative of Gly, and a derivative of Lys. In some embodiments, each A¹¹ is Glu. In some embodiments, each A¹¹ is Gln. In some embodiments, each A¹¹ is Gly. In some embodiments, each A¹¹ is Lys. In some embodiments, each A¹¹ is a derivative of Glu. In some embodiments, each A¹¹ is a derivative of Gln. In some embodiments, each A¹¹ is a derivative of Gly. In some embodiments, each A¹¹ is a derivative of Lys.

In some embodiments, m is an integer from 1 to 10. In some embodiments m is an integer from 2 to 8. In some embodiments, m is an integer from 1 to 9. In some embodiments, m is an integer from 2 to 10. In some embodiments, m is an integer from 1 to 8. In some embodiments, m is an integer from 3 to 10. In some embodiments, m is an integer from 1 to 7. In some embodiments, m is an integer from 4 to 10. In some embodiments, m is an integer from 1 to 6.

In some embodiments, m is 1, 2, 3, 4, 5, 6, 7, 8, 9, or 10. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

L¹ may be any of the linkers described above, any linker known in the art, or a combination thereof. In some embodiments, L¹ is a combination of at least two of the previously described linkers. In some embodiments, L¹ is a combination of two of the previously described linkers. In some embodiments, L¹ is a combination of two or more of the previously described linkers.

In some embodiments, L¹ is a linker connected to a side chain of an amino acid, wherein the amino acid and L¹ together have the following structure:

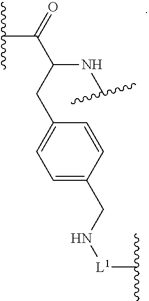

In some embodiments, L¹ is a linker connected to a side chain of an amino acid, wherein the amino acid and L¹ together have the following structure:

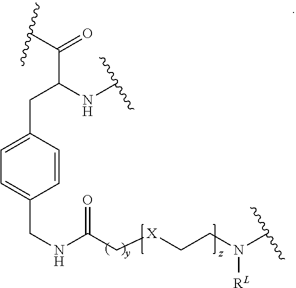

In some embodiments, L¹ is a linker connected to a side chain of an amino acid, wherein the amino acid and L¹ together have the following structure:

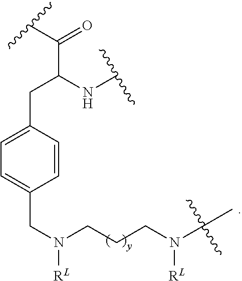

In further embodiments, L¹ is a linker connected to a side chain of an amino acid, wherein the amino acid and L¹ together have the following structure:

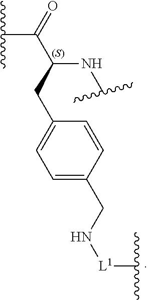

In some embodiments, L¹ is a linker connected to a side chain of an amino acid, wherein the amino acid and L¹ together have the following structure:

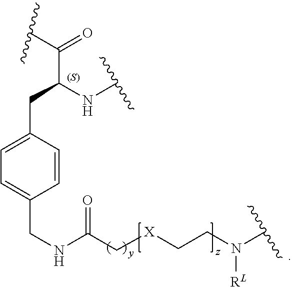

In some embodiments, L¹ is a linker connected to a side chain of an amino acid, wherein the amino acid and L¹ together have the following structure:

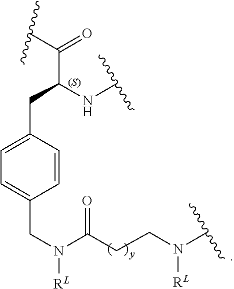

In one or more embodiments, L¹ is a linker connected to a side chain of an amino acid, wherein the amino acid and L¹ together have the following structure:

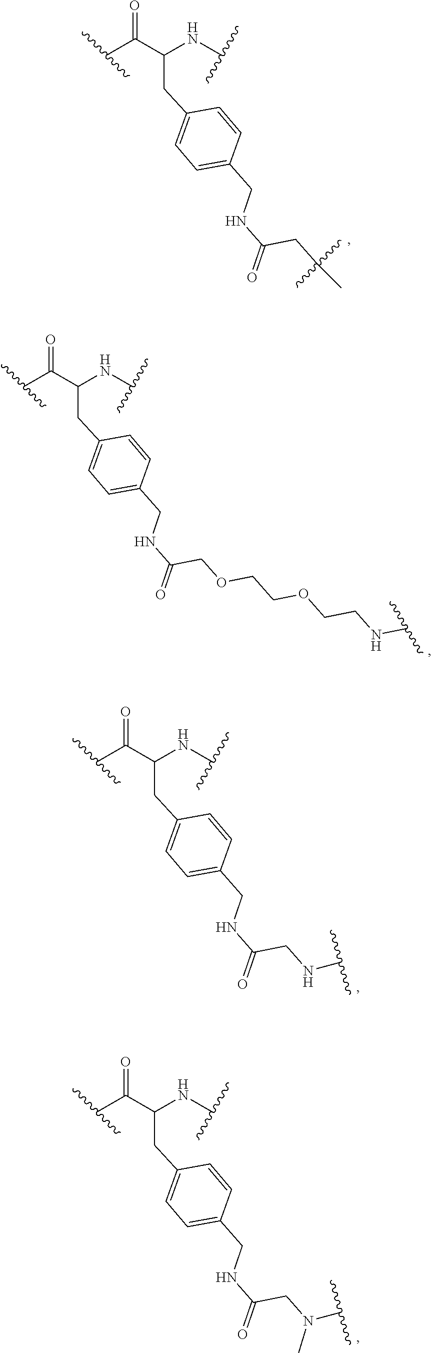 [SMILES: *CC(=O)NCc1ccc(CC(N*)C(*)=O)cc1 ; *NCCOCCOCC(=O)NCc1ccc(CC(N*)C(*)=O)cc1 ; *NCC(=O)NCc1ccc(CC(N*)C(*)=O)cc1 ; *NC(Cc1ccc(CNC(=O)CN(*)C)cc1)C(*)=O]

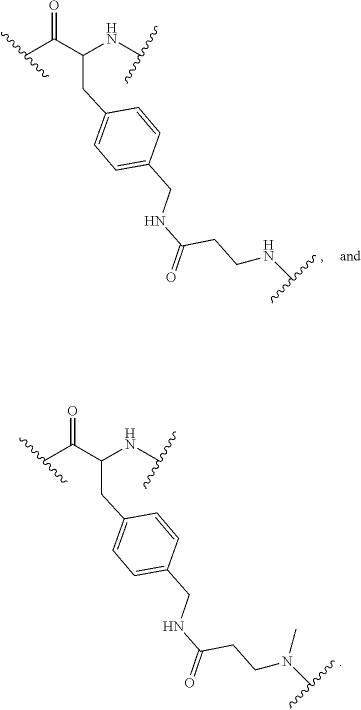 [SMILES: *NCCC(=O)NCc1ccc(CC(N*)C(*)=O)cc1 ; *NC(Cc1ccc(CNC(=O)CCN(*)C)cc1)C(*)=O]

In particular embodiments, L¹ is a linker connected to a side chain of an amino acid, wherein the amino acid and L¹ together have the following structure:

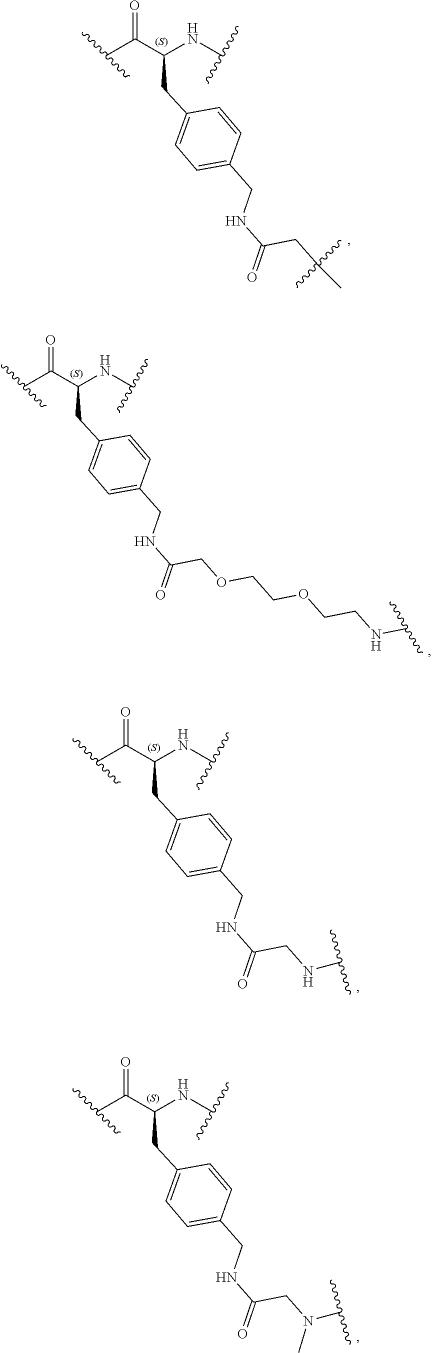

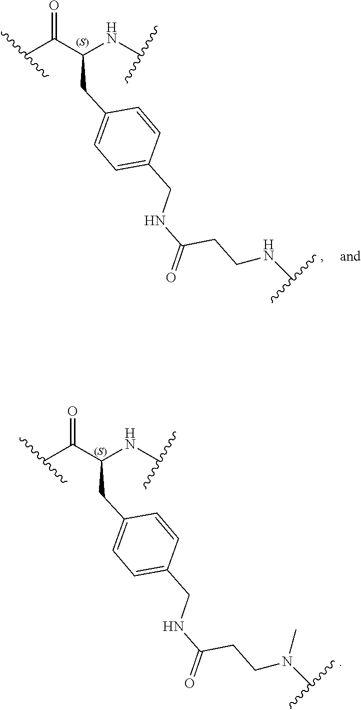

In some embodiments, L¹ is a linker connected to a side chain of A⁴. In some embodiments, L¹ is a linker connected to a side chain of A⁴, wherein A⁴ and L¹ together have a structure selected from:

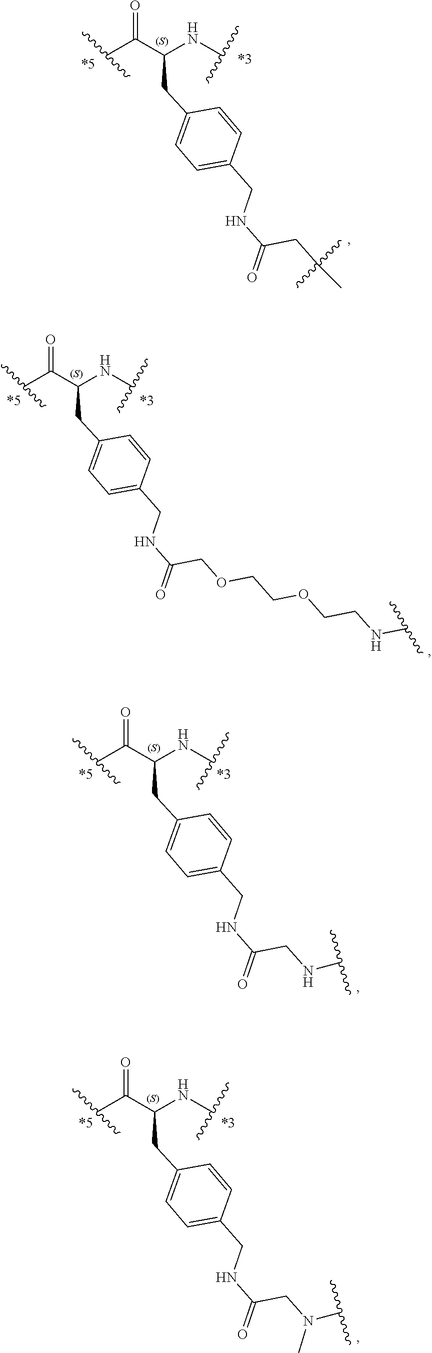

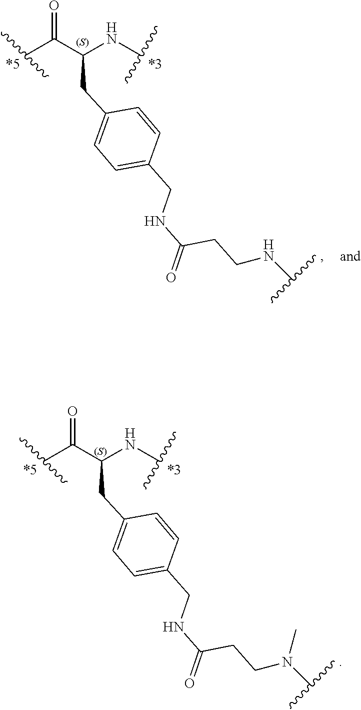

In some embodiments, L¹ is a linker connected to a side chain of an amino acid, wherein the amino acid and L¹ together have the following structure:

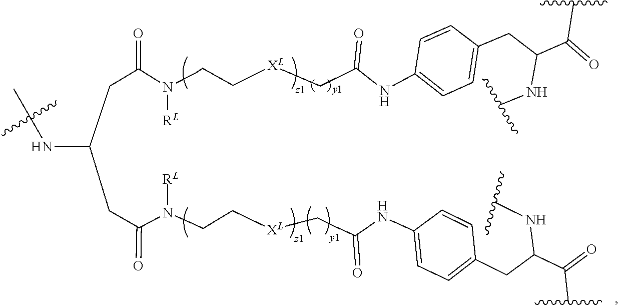

wherein:
X^{L} is CH₂, N, or O;
R^{N} is H, C₁₋₃-alkyl, or acyl; and
z1, independently at each occurrence, is 0, 1, 2, 3, or 4.

In some embodiments, L¹ is a linker connected to a side chain of an amino acid, wherein the amino acid and L¹ together have the following structure:

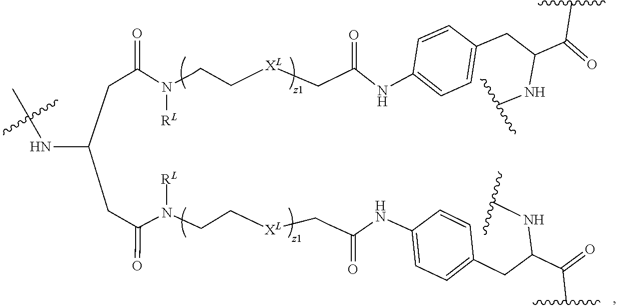

wherein:
X^{L} is CH₂, N, or O;
R^{N} is H, C₁₋₃-alkyl, or acyl;
y1, independently, at each occurrence, is 0, 1, 2, 3, 4, 5, or 6; and
z1, independently at each occurrence, is 0, 1, 2, 3, or 4.

In some embodiments, L¹ is a linker connected to a side chain of an amino acid, wherein the amino acid and L¹ together have the following structure:

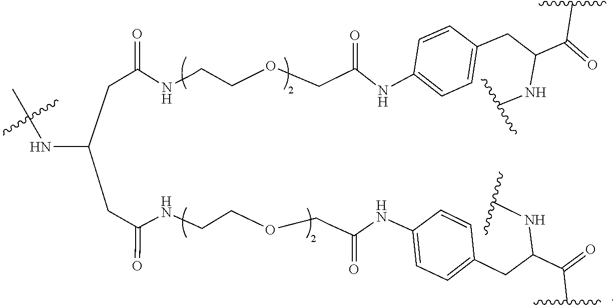

In some embodiments, L¹ is a linker connected to a side chain of an amino acid, wherein the amino acid and L¹ together have the following structure:

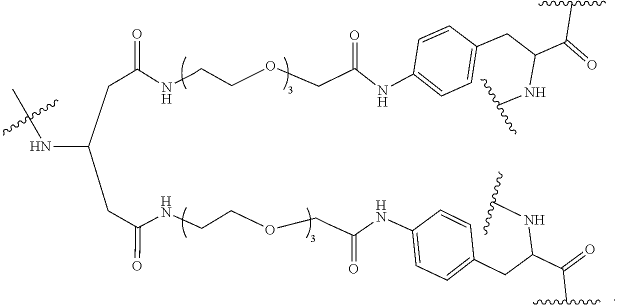

### M (e.g., of Compounds of Formulae (I), (Ia), (Ib), (Ic), and (Id))

As disclosed herein, M can be an imaging agent, a chelating agent optionally radiolabeled with a radionuclide, or a radionuclide. In some embodiments, M is an imaging agent. In some embodiments, M is a chelator. In some embodiments, M is a chelator radiolabeled with a radionuclide. In some embodiments, M is a radionuclide. Any M may be connected to the cyclic peptide via any L¹ described above (whether L¹ is a linker or a bond). The skilled artisan would be able to determine suitable combinations of M and L¹ based on the present disclosure, or suitable substitution of the cyclic peptide directly with M in the case where L¹ is a bond.

### Imaging Agents

In one or more embodiments of the FAP-targeting compounds described herein, M is an imaging agent. FAP-targeting compounds that include an imaging agent may be useful for in vitro and/or in vivo visualization of FAP-related disease progression or response after receiving the instantly disclosed compounds. In some embodiments, the imaging agent is a non-radioactive (or non-radionuclide) imaging agent. In some embodiments, the imaging agent is a fluorescent imaging agent.

Suitable imaging agents that may be connected to the cyclic peptide include Cy dyes, Culfo Cy dyes, Alexa Fluor dyes, Dylight dyes, FluoProbes dyes, Seta dyes, IRIS dyes, and other dyes that can be used interchangeably with Cy dyes in most biochemical imaging applications. For example, the compounds of the instant disclosure may be connected to imaging, but not limited to, Cy2, Cy3, Cy3.5, Cy5, Cy5.5, Cy7, Cy7.5, Sulfo Cy2, Sulfo Cy3, Sulfo Cy3.5, Sulfo Cy5, Sulfo Cy5.5, Sulfo Cy7, Sulfo Cy7.5, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Dylight 350, Dylight 405, Dylight 488, Dylight 550, Dylight 594, Dylight 633, Dylight 650, Dylight 680, Fluorprobes 390, Fluorprobes 488, Fluorprobes 532, Fluorprobes 547H, Fluorprobes 594, Fluorprobes 647H, Fluorprobes 682, Seta 375 NHS, Seta 400 NHS, Seta 405 NHS, Seta 475 Maleimide, Seta 470 NHS, Seta 555 Azide, Seta 555 DBCO, Seta 555 NHS, Seta 632 Maleimide, Seta 632 NHS, Seta 633 Azide, Seat 633 NHS, Seta 635 pH di-NHS, Seta 640 pH di-NHS, Seta 646, Maleimide, Seta 646 NHS, Seta 650 Azide, Seta 650 Maleimide, IRIS 2, IRIS 3, IRIS, 3.5, IRIS 5, IRIS 5.5, IRIS 7, IRIS 2 NHS Active, IRIS 3 NHS Active, IRIS 3.5 NHS Active, IRIS 5 NHS Active, IRIS 5.5 NHS Active, and IRIS 7 NHS Active.

Additional exemplary imaging agents that may be incorporated into a compound of the present disclosure includes those described in Elmes, R. Bioreductive fluorescent imaging agents: applications in tumor hypoxia. Chem. Commun. 2016, 52, 8935, and Schouw et al. Targeted optical fluorescence imaging: a meta-narrative review and future perspectives. Eur. J Nucl. Med. Mol. Imaging. 2021 December; 48(13):4272-4292.

In some embodiments, the imaging agent is selected from Cy2, Cy3, Cy3.5, Cy5, Cy5.5, Cy7, Cy7.5, Sulfo Cy2, Sulfo Cy3, Sulfo Cy3.5, Sulfo Cy5, Sulfo Cy5.5, Sulfo Cy7, and Sulfo Cy7.5. In some embodiments, the imaging agent is selected from Cy3, Cy3.5, Cy5, Cy5.5, Cy7, Sulfo Cy3, Sulfo Cy3.5, Sulfo Cy5, Sulfo Cy5.5, and Sulfo Cy7. In some embodiments, the imaging agent is selected from Cy3, Cy3.5, Cy5, Cy5.5, Sulfo Cy3, Sulfo Cy3.5, Sulfo Cy5, and Sulfo Cy5.5. In some embodiments, the imaging agent is selected from Cy5, Cy5.5, Sulfo Cy5, and Sulfo Cy5.5. In some embodiments, the imaging agent is Cy5 or Sulfo Cy5.

In some embodiments, FAP-targeting compounds are connected to Sulfo Cy5:

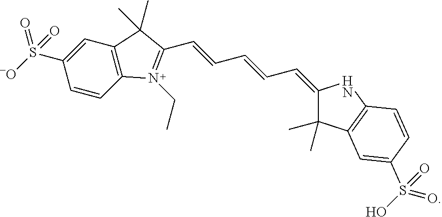

In some embodiments, M is an imaging agent attached to L¹, and L¹ is a linker attached to a side chain of an amino acid of the cyclic peptide (P). In further embodiments, M is an imaging agent, and L¹ is a linker attached to a side chain NH₂ of an amino acid of the cyclic peptide. In still further embodiments, M is an imagining agent and L¹ is a linker attached to a side chain NH₂ of A¹ or A⁸ of the cyclic peptide. In yet further embodiments, M is an imagining agent and L¹ is a linker attached to a lysine side chain of the cyclic peptide, wherein the lysine is at position A¹ or A⁸.

In certain embodiments of FAP-targeting compounds of the present disclosure, M is an imaging agent and L¹ is a linker attached to a side chain of A¹ or A⁸, where M and L¹, together with A¹ or A⁸ have the structure:

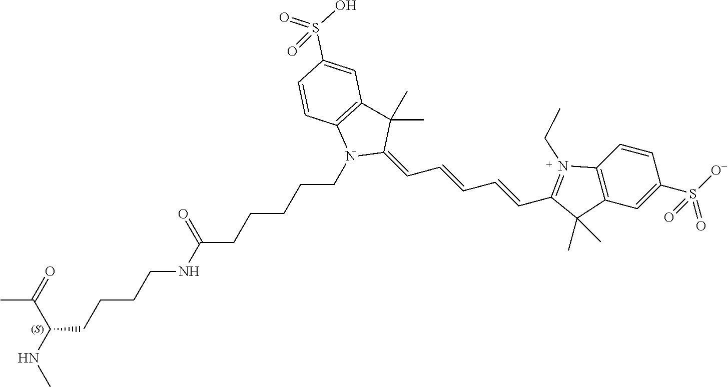

In some embodiments, the compounds disclosed herein comprise both a radionuclide, which may be suitable for imaging (i.e., diagnostically active) or therapy, and a non-radioactive imaging agent.

### Chelators

In one or more embodiments of the FAP-targeting compounds described herein, M is a chelator. As used herein, the terms “chelator,” “chelating ligand,” and “chelating agent” are interchangeable and typically refer to chemical moieties, agents, compounds, or molecules able to form a complex containing one or more coordinate bonds with a metal ion. The chelators described herein may form a complex with any metal element or metal ion known in the art (e.g. any element of ion of alkali metals, alkaline earth metals, transition metals, inner transition metals (lanthanides, actinides), metalloids, or poor metals, said metals being present alone or in connection with another element, e.g. an ion of a metal in connection with a halogen, e.g. a metal-halogenide) to coordinate to a chelating agent. In some embodiments, the chelator is a nuclide chelator. In some embodiments, the chelator is a radionuclide chelator.

Chelators that form a complex with a metal ion, such as a radionuclide, may be referred to herein as radiolabeled chelators. In some embodiments, a chelator complexed or coordinated to a metal ion is depicted with single bonds between heteroatoms of the chelator and the metal ion or is depicted with dashed bonds between the heteroatoms of the chelator and the metal ion.

In some embodiments, the chelator forms a complex with a metal ion and with one or more additional molecules, for example, a solvent molecule (so as to form a solvate), (e.g., a water molecule (so as to form a hydrate)).

When the chelator is chelated or complexed to a radionuclide, it can be said that the chelator is radiolabeled. In some embodiments of the compounds described herein, M is a chelator, and the resulting conjugate of the cyclic peptide with a chelator and optionally a linker can be radiolabeled with a radionuclide.

In some embodiments, chelating agents suitable for coordinating to metal ions may have a cyclic or acyclic structure. In some embodiments, chelating agents suitable for coordinating to metal ions may have a cyclic or linear structure.

In some embodiments, the chelator is a cyclic chelator. As used herein, the term “cyclic chelator” means any chelator having a heterocyclic ring in which two or more of the ring heteroatoms form a coordinate covalent bond to a metal ion, such as, for example, a radionuclide. In an embodiment, the heterocyclic ring has at least two heteroatoms selected from the group consisting of nitrogen, oxygen, and sulfur. In another embodiment, the heterocyclic ring is a macrocycle having between 7 and 30 ring atoms. In yet another embodiment, the heterocyclic ring is a macrocycle having between 7 and 20 ring atoms. In still another embodiment, the heterocyclic ring is a macrocycle having between 7 and 15 ring atoms. In an embodiment, the cyclic chelator is DOTA.

In some embodiments, the chelator is an acyclic chelator. As used herein, the term “acyclic chelator” means any chelator having an open chain that contains heteroatoms in which two or more of the heteroatoms form a coordinate covalent bond to a metal ion, such as, for example, a radionuclide. An acyclic chelator may comprise cycloalkyl, heterocyclic, aryl, and heteroaryl rings, or a combination thereof. However, when the acyclic chelator comprises heterocyclic and/or heteroaryl rings, no more than one heteroatom of each ring participates in the coordinate covalent bond with the metal ion. In an embodiment, the acyclic chelator is a linear chelator, such as, for example, H₂dedpa, BFO, DFO, and mertiatide. In another embodiment, the acyclic chelator is a branched chelator, such as, for example, EDTA, DTPA, Pip-DTPA, ECC, ECD, and citric acid.

Suitable cyclic chelators may have a heterocyclic core, such as, for example, a crown ether or an aza-crown ether. In an embodiment, the heterocyclic core is an aza-crown ether. In another embodiment, the heterocyclic core comprises nitrogen and oxygen heteroatoms. In yet another embodiment, the heterocyclic core is a crown ether.

Chelators in accordance with the present disclosure may have a heterocyclic core comprising from 2 to 10 heteroatoms. For example, the chelator may have a heterocyclic core comprising 2, 3, 4, 5, 6, 7, 8, 9, or 10 heteroatoms. In some embodiments, heterocyclic core comprises from 2 to 8 heteroatoms, from 3 to 8 heteroatoms, from 2 to 6 heteroatoms or from 3 to 6 heteroatoms. In an embodiment, the heterocyclic core comprises 3 heteroatoms. In another embodiment, the heterocyclic core comprises 4 heteroatoms. In another embodiment, the heterocyclic core comprises 5 heteroatoms. In yet another embodiment, the heterocyclic core comprises 6 heteroatoms.

In some embodiments, one or more heteroatoms of the core structure is functionalized. For example, heteroatoms of the cyclic core may be functionalized with one or more of H, OH, C(O)OR^{x}, CR^{x}₂C(O)R^{x}, (CR^{x})ₙC(O)OR^{x}, (CR^{x})ₙC(O)NH₂, (CR^{x})ₙC(O)NHR^{x}, (CR^{x})ₙC(O)NR^{x}₂, CR^{x}₂P(O)(OR^{x})₂, (CR^{x})ₙP(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SCN, C(O)OR^{x}, C(O)NR^{x}, P(O)(OR^{x})₂, N₃, NR^{x}₂, SR^{x}, halo, alkyl, haloalkyl, aryl, heteroaryl, benzyl, where alkyl, haloalkyl, aryl, heteroaryl, and benzyl are optionally substituted with one or more R, and each R is independently selected from H, OH, CN, SCN, NH₂, CH₂C(O)OH, C(O)OH, halo, alkyl, haloalkyl, alkylaryl, heteroaryl, cycloalkyl, heterocycloalkyl, and P(O)(OH)₂ and n is 0, 1, 2, 3, 4, or 5.

In some embodiments, the heterocyclic core of the chelator comprises heteroatoms separated by alkylene groups, such as, for example, methylene, ethylene, or propylene groups. In some embodiments, the heteroatoms of the heterocyclic core are separated by ethylene groups. In some embodiments, the heteroatoms of the heterocyclic core are separated by methylene groups. The alkylene groups separating the heteroatoms of the core structure may be substituted or unsubstituted. In some embodiments, one or more alkylene groups of the heterocyclic core are substituted with one or more H, OH, C(O)OR^{x}, CR^{x}₂C(O)OR^{x}, CR^{x}₂P(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SCN, C(O)OR^{x}, C(O)NR^{x}, P(O)(OR^{x})₂, N₃, NR^{x}₂, SR^{x}, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SCN, NH₂, CH₂C(O)OH, C(O)OH, halo, alkyl, alkoxy, haloalkyl, alkylaryl, heteroaryl, cycloalkyl, heterocycloalkyl, P(O)(OH)₂, and SH.

In an embodiment, the cyclic chelator comprises a 1,4,7,10-tetraazacyclododecane core or a residue thereof, such as, for example, the structures shown in Table 3 or residues thereof, below.

TABLE 3
Exemplary Cyclic Chelators with a 1,4,7,10-tetraazacyclododecane core

| | |
|---|---|
| DOTA | 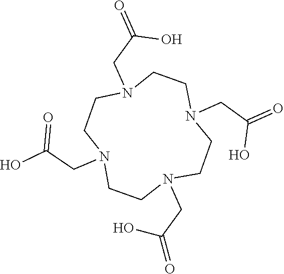 [SMILES: O=C(O)CN1CCN(CC(=O)O)CCN(CC(=O)O)CCN(CC(=O)O)CC1] |
| DOTMP | 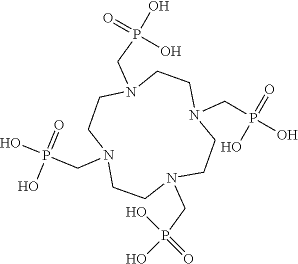 [SMILES: O=P(O)(O)CN1CCN(CP(=O)(O)O)CCN(CP(=O)(O)O)CCN(CP(=O)(O)O)CC1] |
| DO3A | 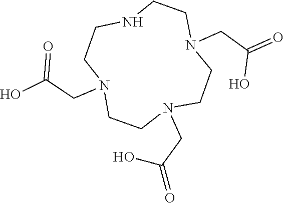 [SMILES: O=C(O)CN1CCNCCN(CC(=O)O)CCN(CC(=O)O)CC1] |
| DOTA-AA | 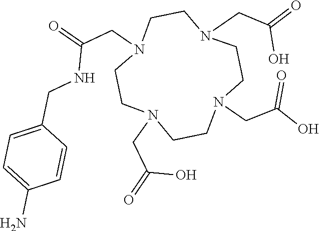 [SMILES: Nc1ccc(CNC(=O)CN2CCN(CC(=O)O)CCN(CC(=O)O)CCN(CC(=O)O)CC2)cc1] |
| DOTASA | 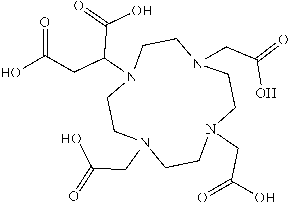 [SMILES: O=C(O)CC(C(=O)O)N1CCN(CC(=O)O)CCN(CC(=O)O)CCN(CC(=O)O)CC1] |
| DOTAZA | 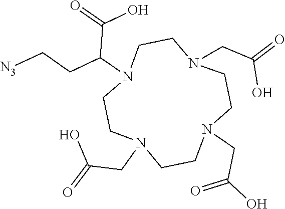 [SMILES: [N-]=[N+]=NCCC(C(=O)O)N1CCN(CC(=O)O)CCN(CC(=O)O)CCN(CC(=O)O)CC1] |
| DOTAGA | 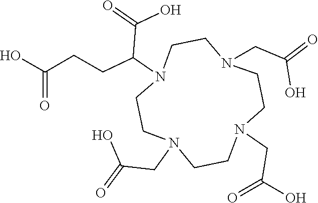 [SMILES: O=C(O)CCC(C(=O)O)N1CCN(CC(=O)O)CCN(CC(=O)O)CCN(CC(=O)O)CC1] |
| | 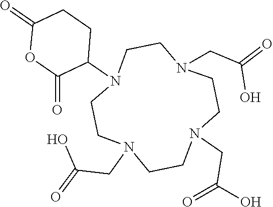 [SMILES: O=C(O)CN1CCN(CC(=O)O)CCN(C2CCC(=O)OC2=O)CCN(CC(=O)O)CC1] |
| Me-DO2PA | 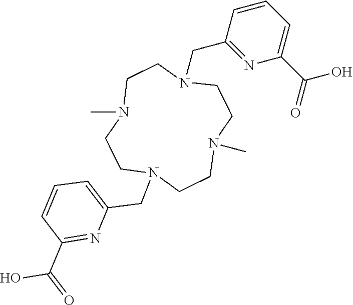 [SMILES: CN1CCN(Cc2cccc(C(=O)O)n2)CCN(C)CCN(Cc2cccc(C(=O)O)n2)CC1] |
| DOTPA | 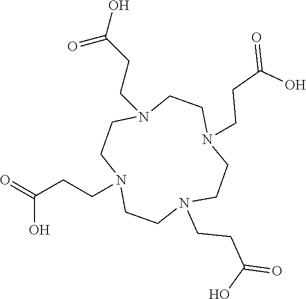 [SMILES: O=C(O)CCN1CCN(CCC(=O)O)CCN(CCC(=O)O)CCN(CCC(=O)O)CC1] |
| DEPA | 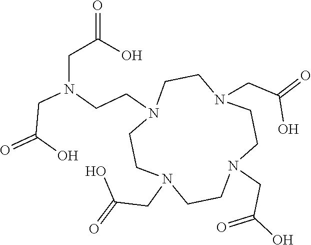 [SMILES: O=C(O)CN1CCN(CCN(CC(=O)O)CC(=O)O)CCN(CC(=O)O)CCN(CC(=O)O)CC1] |
| p-SCN-Bn-DOTA | 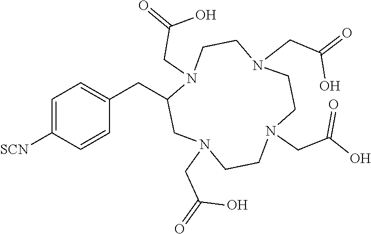 |
| C-DEPA | 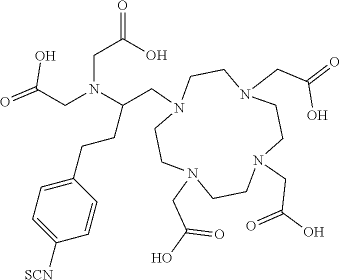 |
| MeO-DOTA-NCS | 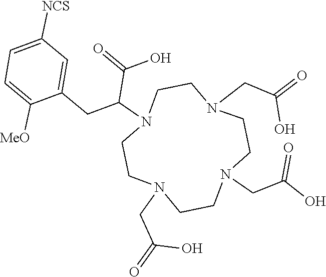 |
| 3p-C-DEPA | 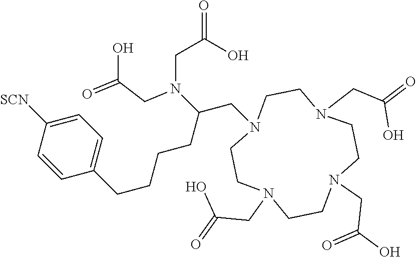 |
| DO3A1P | 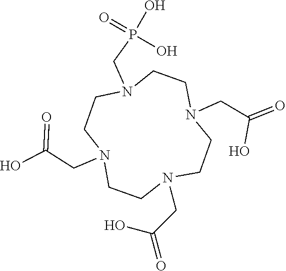 [SMILES: O=C(O)CN1CCN(CC(=O)O)CCN(CP(=O)(O)O)CCN(CC(=O)O)CC1] |
| 3p-C-DE4TA | 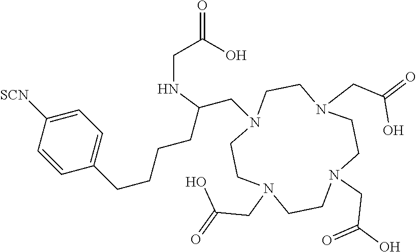 |
| DE4TA | 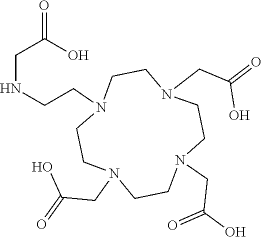 [SMILES: O=C(O)CNCCN1CCN(CC(=O)O)CCN(CC(=O)O)CCN(CC(=O)O)CC1] |
| DODASA | 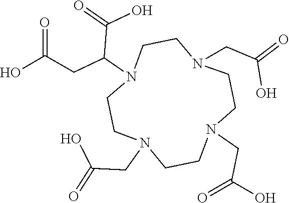 [SMILES: O=C(O)CC(C(=O)O)N1CCN(CC(=O)O)CCN(CC(=O)O)CCN(CC(=O)O)CC1] |
| PCS | 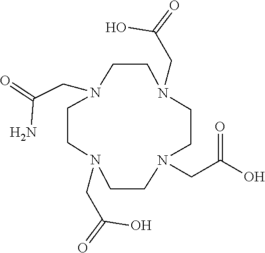 [SMILES: NC(=O)CN1CCN(CC(=O)O)CCN(CC(=O)O)CCN(CC(=O)O)CC1] |
| DOTAM (also referred to as TCMC) | 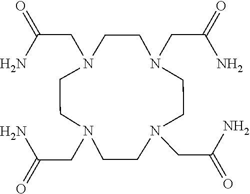 [SMILES: NC(=O)CN1CCN(CC(N)=O)CCN(CC(N)=O)CCN(CC(N)=O)CC1] |

In another embodiment, the cyclic chelator comprises a 1,4,7-triazacyclononane core, such as, for example, the structures shown in Table 4, below.

TABLE 4
Exemplary Cyclic Chelators with a 1,4,7-triazacyclononane core

| | |
|---|---|
| NOTA | 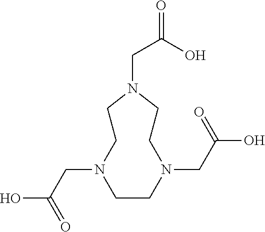 [SMILES: O=C(O)CN1CCN(CC(=O)O)CCN(CC(=O)O)CC1] |
| TRAP | 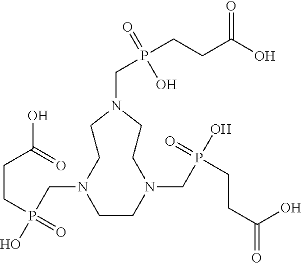 [SMILES: O=C(O)CCP(=O)(O)CN1CCN(CP(=O)(O)CCC(=O)O)CCN(CP(=O)(O)CCC(=O)O)CC1] |
| NS3 | 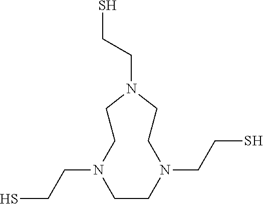 [SMILES: SCCN1CCN(CCS)CCN(CCS)CC1] |
| NETA | 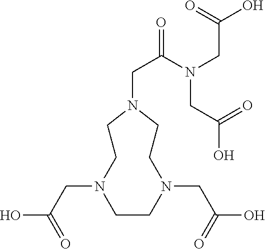 [SMILES: O=C(O)CN1CCN(CC(=O)O)CCN(CC(=O)N(CC(=O)O)CC(=O)O)CC1] |
| NODAGA | 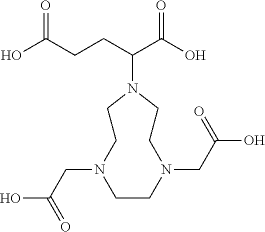 [SMILES: O=C(O)CCC(C(=O)O)N1CCN(CC(=O)O)CCN(CC(=O)O)CC1] |
| C-NETA | 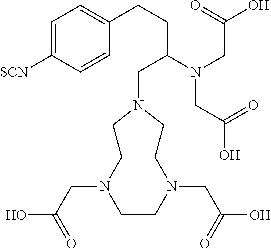 |
| p-SCN- Bn-NOTA | 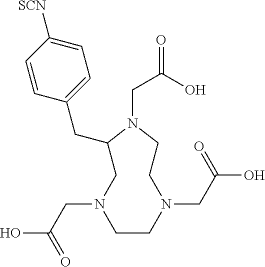 |
| 3p-C- NETA | 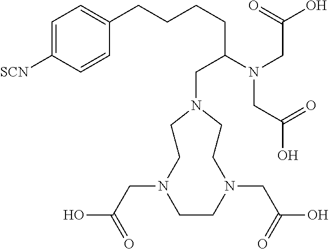 |
| NO2A | 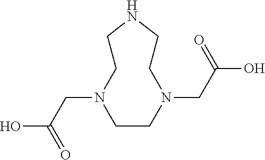 [SMILES: O=C(O)CN1CCNCCN(CC(=O)O)CC1] |
| NESTA | 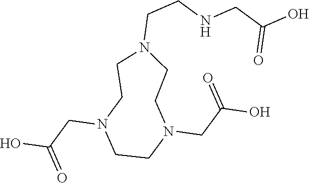 [SMILES: O=C(O)CNCCN1CCN(CC(=O)O)CCN(CC(=O)O)CC1] |
| NO2AP | 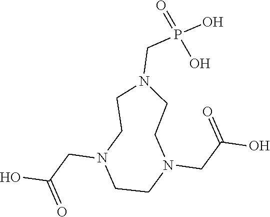 [SMILES: O=C(O)CN1CCN(CC(=O)O)CCN(CP(=O)(O)O)CC1] |
| C-NESTA | 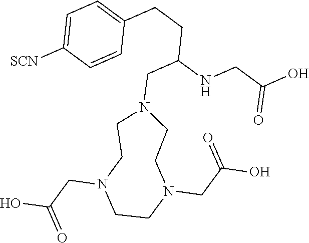 |
| NOTP | 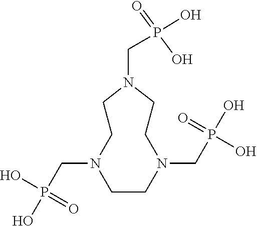 [SMILES: O=P(O)(O)CN1CCN(CP(=O)(O)O)CCN(CP(=O)(O)O)CC1] |
| 3p-C- NESTA | 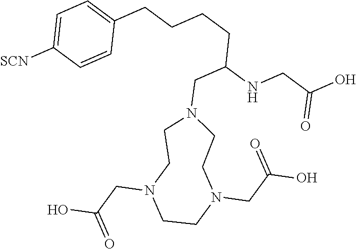 |
| NOPO | 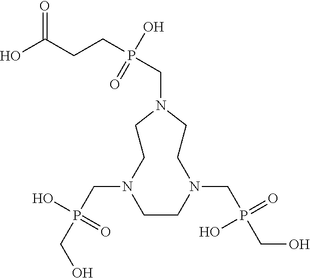 [SMILES: O=C(O)CCP(=O)(O)CN1CCN(CP(=O)(O)CO)CCN(CP(=O)(O)CO)CC1] |
| NODIA- Me | 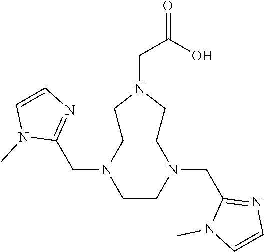 [SMILES: Cn1ccnc1CN1CCN(CC(=O)O)CCN(Cc2nccn2C)CC1] |

In some embodiments, the cyclic chelator comprises a bridged heterocyclic core. In some embodiments, the cyclic chelator comprises a fused heterocyclic core. For example, in one or more embodiments, the heterocyclic core (of the cyclic chelator) may be fused to a heteroaryl or heterocycloalkyl ring, such that the two cycles share a heteroatom. Suitable heteroaryl and heterocycloalkyl rings that may be fused to the heterocyclic core (of the cyclic chelator) include, but are not limited to pyridine, pyrimidine, furan, tetrahydropyran, pyran, dioxane, and oxazole, imidazole, and pyrrole.

Chelators comprising a fused or bridged core structure, as well as various other suitable heterocyclic cores, include, but are not limited to, the structures shown in Table 5, below.

TABLE 5
Additional Exemplary Cyclic Chelators

| | |
|---|---|
| CB-DO2A | 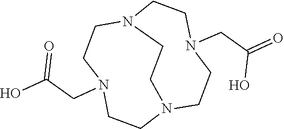 [SMILES: O=C(O)CN1CCN2CCN(CC1)CCN(CC(=O)O)CC2] |
| L3 | 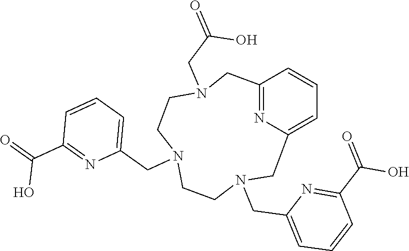 [SMILES: O=C(O)CN1CCN(Cc2cccc(C(=O)O)n2)CCN(Cc2cccc(C(=O)O)n2)Cc2cccc(n2)C1] |
| TRITA | 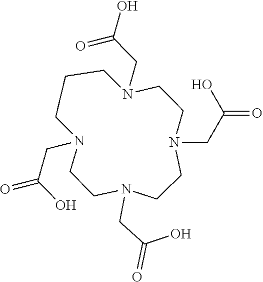 [SMILES: O=C(O)CN1CCCN(CC(=O)O)CCN(CC(=O)O)CCN(CC(=O)O)CC1] |
| H2macropa | 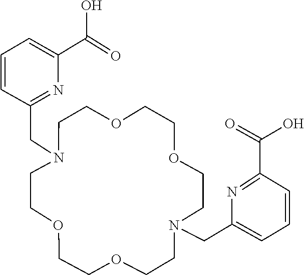 [SMILES: O=C(O)c1cccc(CN2CCOCCOCCN(Cc3cccc(C(=O)O)n3)CCOCCOCC2)n1] |
| TETA | 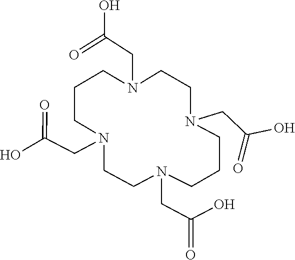 [SMILES: O=C(O)CN1CCCN(CC(=O)O)CCN(CC(=O)O)CCCN(CC(=O)O)CC1] |
| p-SCN-Bn-macropa | 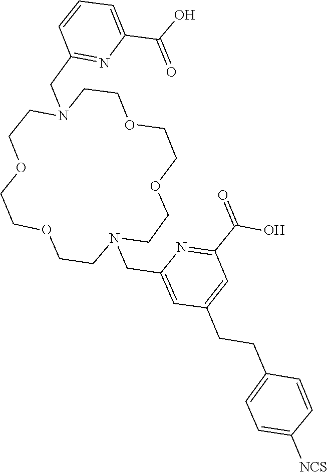 |
| TE2A | 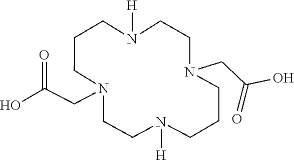 [SMILES: O=C(O)CN1CCCNCCN(CC(=O)O)CCCNCC1] |
| PEPA | 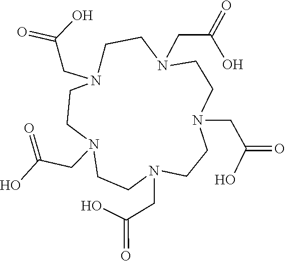 [SMILES: O=C(O)CN1CCN(CC(=O)O)CCN(CC(=O)O)CCN(CC(=O)O)CCN(CC(=O)O)CC1] |
| TETPA | 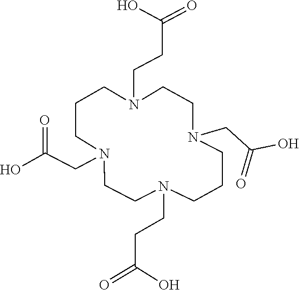 [SMILES: O=C(O)CCN1CCCN(CC(=O)O)CCN(CCC(=O)O)CCCN(CC(=O)O)CC1] |
| p-SCN-Bn-PEPA | 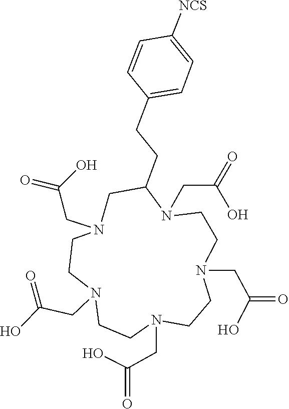 |
| PCTA | 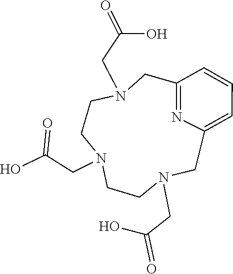 [SMILES: O=C(O)CN1CCN(CC(=O)O)Cc2cccc(n2)CN(CC(=O)O)CC1] |
| HEHA | 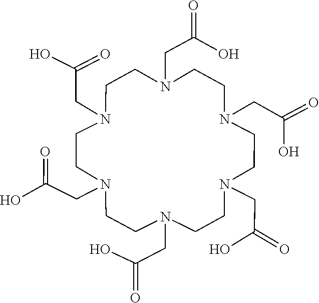 [SMILES: O=C(O)CN1CCN(CC(=O)O)CCN(CC(=O)O)CCN(CC(=O)O)CCN(CC(=O)O)CCN(CC(=O)O)CC1] |
| p-SCN-Bn-PCTA | 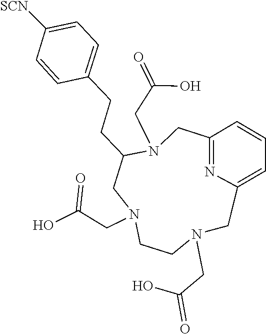 |
| p-SCN-Bn-HEHA | 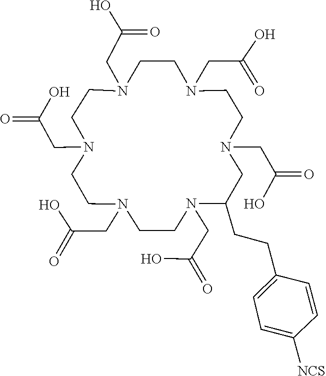 |
| L1 | 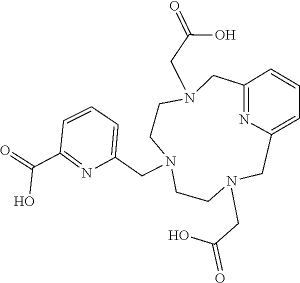 [SMILES: O=C(O)CN1CCN(Cc2cccc(C(=O)O)n2)CCN(CC(=O)O)Cc2cccc(n2)C1] |
| FSC | 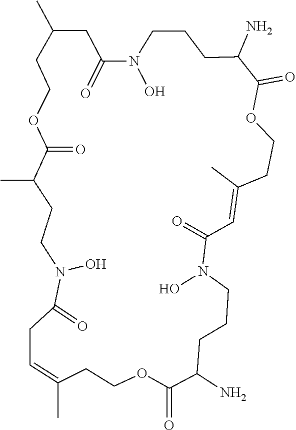 [SMILES: C/C1=C/CC(=O)N(O)CCC(C)C(=O)OCCC(C)CC(=O)N(O)CCCC(N)C(=O)OCC/C(C)=C/C(=O)N(O)CCCC(N)C(=O)OCC1] |
| L2 | 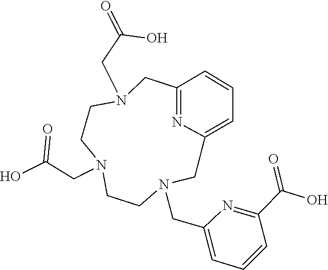 [SMILES: O=C(O)CN1CCN(CC(=O)O)Cc2cccc(n2)CN(Cc2cccc(C(=O)O)n2)CC1] |
| AAZTA | 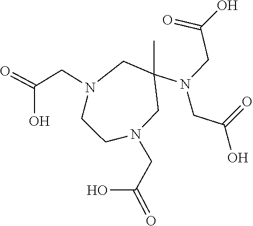 [SMILES: CC1(N(CC(=O)O)CC(=O)O)CN(CC(=O)O)CCN(CC(=O)O)C1] |
| CNAAZTA | 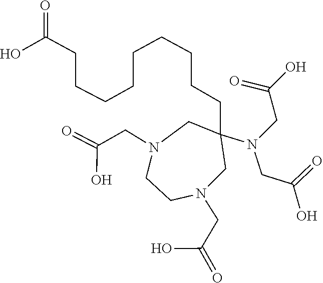 [SMILES: O=C(O)CCCCCCCCCC1(N(CC(=O)O)CC(=O)O)CN(CC(=O)O)CCN(CC(=O)O)C1] |
| DATA^{m} | 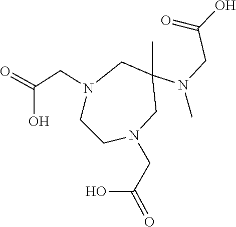 [SMILES: CN(CC(=O)O)C1(C)CN(CC(=O)O)CCN(CC(=O)O)C1] |
| AAZTA5 | 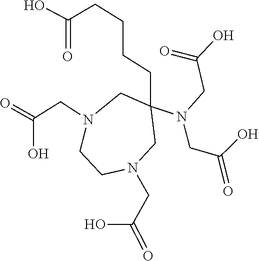 [SMILES: O=C(O)CCCCC1(N(CC(=O)O)CC(=O)O)CN(CC(=O)O)CCN(CC(=O)O)C1] |
| H₂bispa² | 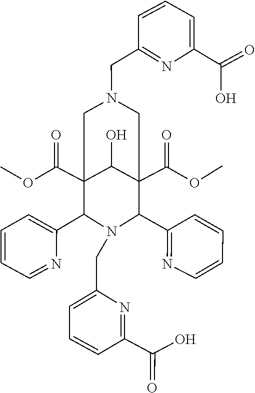 [SMILES: COC(=O)C12CN(Cc3cccc(C(=O)O)n3)CC(C(=O)OC)(C(c3ccccn3)N(Cc3cccc(C(=O)O)n3)C1c1ccccn1)C2O] |
| bispidine | 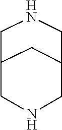 |
| bispidine-L₁ | 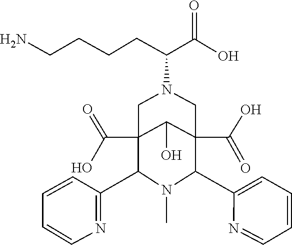 [SMILES: CN1C(c2ccccn2)C2(C(=O)O)CN([C@H](CCCCN)C(=O)O)CC(C(=O)O)(C1c1ccccn1)C2O] |
| CB-TE2A | 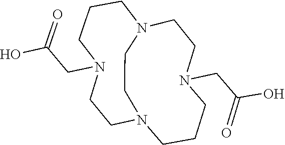 [SMILES: O=C(O)CN1CCCN2CCN(CCCN(CC(=O)O)CC2)CC1] |
| Sarcophagine | 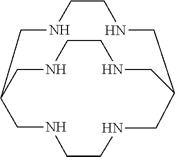 [SMILES: C1CNCC2CNCCNCC(CN1)CNCCNC2] |
| macropa-XL | 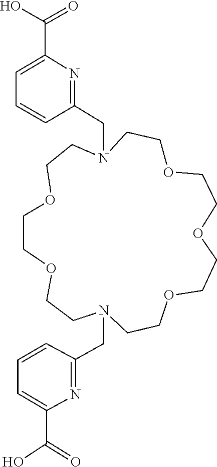 [SMILES: O=C(O)c1cccc(CN2CCOCCOCCOCCN(Cc3cccc(C(=O)O)n3)CCOCCOCC2)n1] |
| DB30C10 | 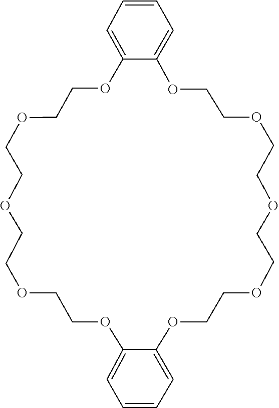 [SMILES: c1ccc2c(c1)OCCOCCOCCOCCOc1ccccc1OCCOCCOCCOCCO2] |

In some embodiments, the chelator is an acyclic or linear chelator. Suitable linear chelating ligands may comprise a series of functionalized amines separated by alkylene, arylene, cycloalkyl, heteroaryl, heterocyclyl groups, or a combination thereof. In some embodiments, the acyclic chelator comprises 2, 3, or 4 functionalized amines. For example, in some embodiments the linear chelator comprises 2, 3, or 4 amines functionalized with one or more of H, OH, C(O)OR^{x}, CR^{x}₂C(O)OR^{x}, CR^{x}₂P(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR, CN, SCN, C(O)OR^{x}, C(O)NR^{x}, P(O)(OR^{x})₂, N₃, NR^{x}₂, SR, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SCN, NH₂, CH₂COOH, C(O)OH, halo, alkyl, haloalkyl, alkylaryl, heteroaryl, cycloalkyl, heterocycloalkyl, and P(O)(OH)₂.

In some embodiments, the acyclic chelator comprises a series of functionalized amines separated by alkylene groups, such as, for example, methylene, ethylene, and propylene groups. The alkylene groups separating the functionalized amines may be substituted or unsubstituted. In some embodiments, one or more of the alkylene groups separating the functionalized amines are substituted with one or more of H, OH, C(O)OR^{x}, CR^{x}₂C(O)OR^{x}, CR^{x}₂P(O)(OR^{x})₂, P(O)(OR^{x})₂, alkyl, aryl, cycloalkyl, heterocycloalkyl, alkylaryl, alkylheteroaryl, and heteroaryl wherein alkyl, aryl, heteroaryl, heterocycloalkyl, alkylaryl, alkylheteroaryl, and cycloalkyl are optionally substituted with one or more H, OH, OR^{x}, CN, SO₃⁻, SCN, C(O)OR^{x}, C(O)NR^{x}, P(O)(OR^{x})₂, N₃, NR^{x}₂, SR^{x}, halo, alkyl, haloalkyl, aryl, heteroaryl, benzyl, where alkyl, haloalkyl, aryl, heteroaryl, and benzyl are optionally substituted with one or more R^{x}, and each R^{x} is independently selected from H, OH, CN, SO₃⁻, SCN, NH₂, CH₂C(O)OH, C(O)OH, halo, alkyl, haloalkyl, alkylaryl, heteroaryl, cycloalkyl, heterocycloalkyl, and P(O)(OH)₂.

In an embodiment, the linear chelator comprises a series of functionalized amines separated by methylene groups. In another embodiment, the acyclic or linear chelator comprises a series of functionalized amines separated by ethylene groups, such as, for example, the structures provided in Table 6, below.

TABLE 6
Exemplary Acylic Chelators

| | |
|---|---|
| EDTA | 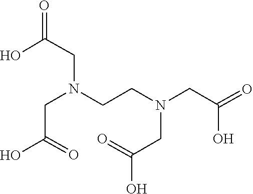 [SMILES: O=C(O)CN(CCN(CC(=O)O)CC(=O)O)CC(=O)O] |
| 1B4M- DTPA | 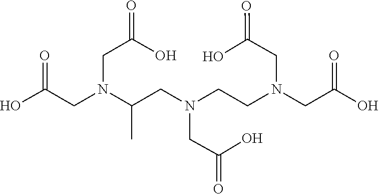 [SMILES: CC(CN(CCN(CC(=O)O)CC(=O)O)CC(=O)O)N(CC(=O)O)CC(=O)O] |
| EDTMP | 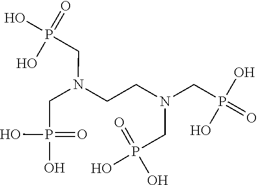 [SMILES: O=P(O)(O)CN(CCN(CP(=O)(O)O)CP(=O)(O)O)CP(=O)(O)O] |
| p-SCN- Bn-DTPA | 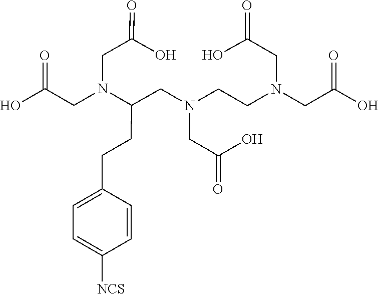 |
| HBED | 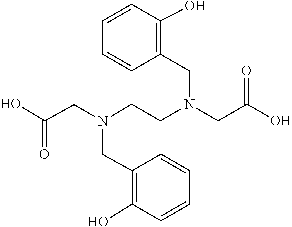 [SMILES: O=C(O)CN(CCN(CC(=O)O)Cc1ccccc1O)Cc1ccccc1O] |
| DTPA- amide | 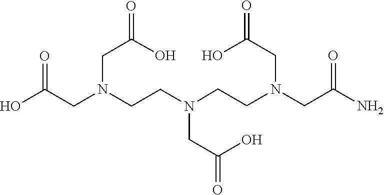 [SMILES: NC(=O)CN(CCN(CCN(CC(=O)O)CC(=O)O)CC(=O)O)CC(=O)O] |
| SHBED | 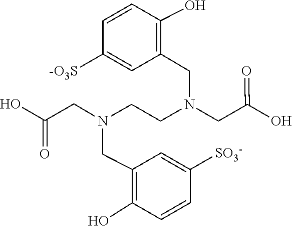 |
| H₄neunpa | 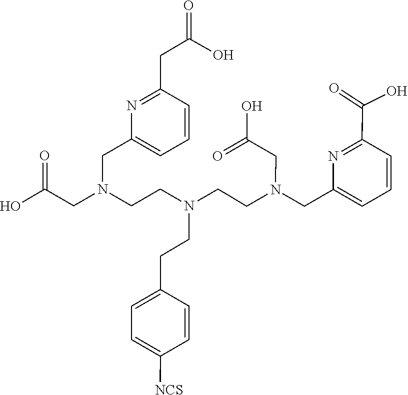 |
| H₆Sbbpen | 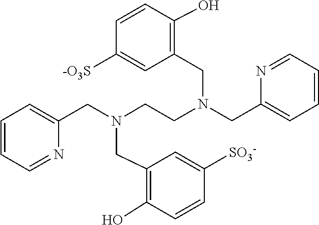 |
| EDTMP | 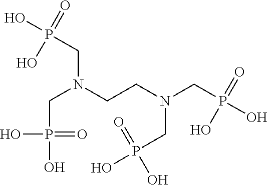 [SMILES: O=P(O)(O)CN(CCN(CP(=O)(O)O)CP(=O)(O)O)CP(=O)(O)O] |
| HBED- CC | 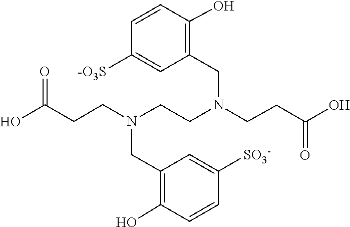 |
| TTHA | 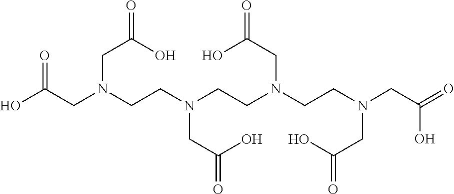 [SMILES: O=C(O)CN(CCN(CCN(CC(=O)O)CC(=O)O)CC(=O)O)CCN(CC(=O)O)CC(=O)O] |
| DTPA | 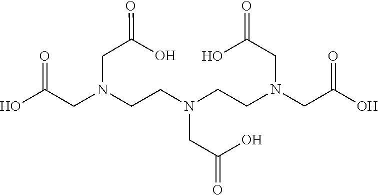 [SMILES: O=C(O)CN(CCN(CC(=O)O)CC(=O)O)CCN(CC(=O)O)CC(=O)O] |
| TTHMP | 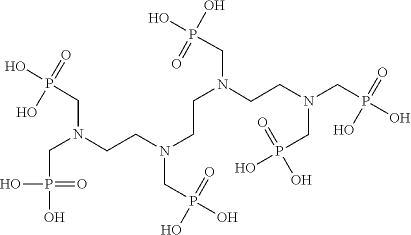 [SMILES: O=P(O)(O)CN(CCN(CCN(CP(=O)(O)O)CP(=O)(O)O)CP(=O)(O)O)CCN(CP(=O)(O)O)CP(=O)(O)O] |

The acyclic chelator may comprise a variety of heteroatoms, such as, for example, nitrogen, oxygen, and sulfur heteroatoms. In some embodiments, the acyclic chelator comprises nitrogen heteroatoms. In some embodiments, the acyclic chelator comprises nitrogen and oxygen heteroatoms. In some embodiments, the acyclic chelator comprises nitrogen and sulfur heteroatoms.

One or more of the heteroatoms of the acyclic or linear chelator may be functionalized. For example, in some embodiments the acyclic or linear chelator comprises one or more heteroatoms functionalized with phosphoric acid, carboxylic acid, substituted or unsubstituted aryl, substituted or unsubstituted phenyl, substituted or unsubstituted heteroaryl, amide, and substituted or unsubstituted alkyl groups. Substituted aryl, benzyl, heteroaryl, and alkyl functionalized amines may be substituted with one or more hydroxyl, carboxyl, alkyl, carbonyl, cyano, phosphoryl, alkoxy, and aryl groups.

One or more heteroatoms of an acyclic chelator may be from a heteroarene, such as, for example, pyridine, pyrrole, thiophene, and furan. One or more heteroatoms of an acyclic chelator may be from a carbonyl functionality, such as, for example, an amide, an ester, an acid, and a thioester. In some embodiments, two or more, but not all, of the heteroatoms in the acyclic core may be connected via a heterocyclyl structure.

In some embodiments, the linear chelator comprises a series of heteroatoms separated by a combination of alkylene and arylene groups. In some embodiments, the linear chelator comprises a series of functionalized amines separated by a combination of alkylene and cycloalkyl groups. Alkylene, arylene, and cycloalkyl groups separating the heteroatoms of a linear chelator may be substituted or unsubstituted. Further suitable acyclic chelating ligands include, but are not limited to, the structures shown in Table 7, below.

TABLE 7
Additional Exemplary Acylic Chelators

| | |
|---|---|
| Pip- DTPA | 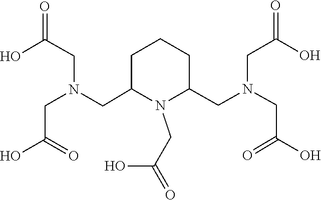 [SMILES: O=C(O)CN(CC(=O)O)CC1CCCC(CN(CC(=O)O)CC(=O)O)N1CC(=O)O] |
| CHX- DTPA | 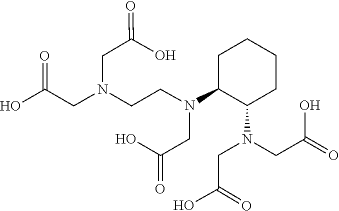 [SMILES: O=C(O)CN(CCN(CC(=O)O)[C@H]1CCCC[C@@H]1N(CC(=O)O)CC(=O)O)CC(=O)O] |
| p-SCN- Bn-CHX- A″-DTPA | 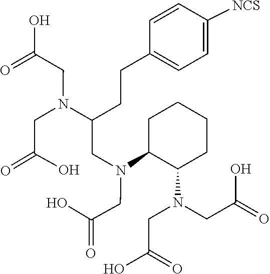 |
| 3,2- HOPO | 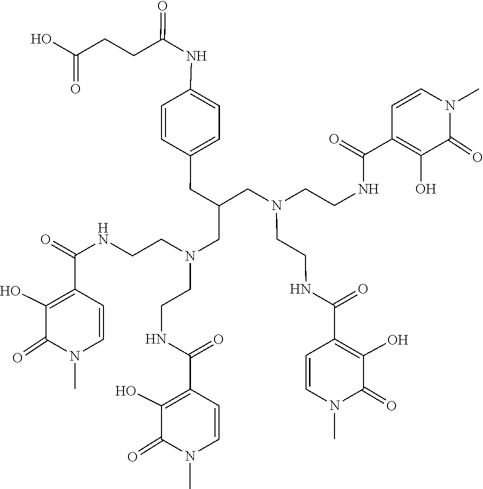 [SMILES: Cn1ccc(C(=O)NCCN(CCNC(=O)c2ccn(C)c(=O)c2O)CC(Cc2ccc(NC(=O)CCC(=O)O)cc2)CN(CCNC(=O)c2ccn(C)c(=O)c2O)CCNC(=O)c2ccn(C)c(=O)c2O)c(O)c1=O] |
| BATPA | 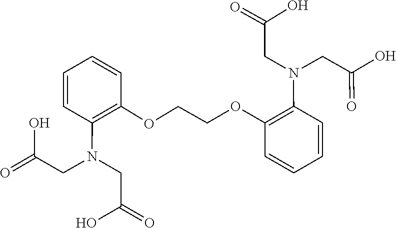 [SMILES: O=C(O)CN(CC(=O)O)c1ccccc1OCCOc1ccccc1N(CC(=O)O)CC(=O)O] |
| THP | 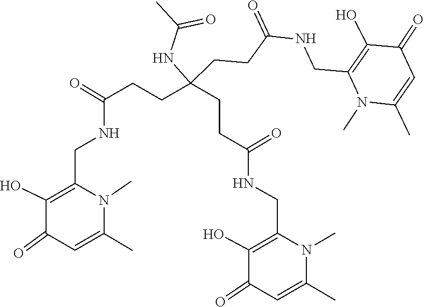 [SMILES: CC(=O)NC(CCC(=O)NCc1c(O)c(=O)cc(C)n1C)(CCC(=O)NCc1c(O)c(=O)cc(C)n1C)CCC(=O)NCc1c(O)c(=O)cc(C)n1C] |
| EGTA | 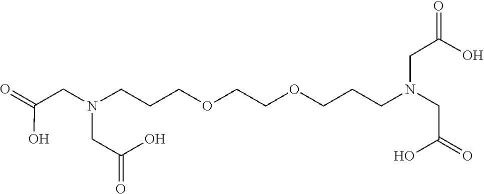 [SMILES: O=C(O)CN(CCCOCCOCCCN(CC(=O)O)CC(=O)O)CC(=O)O] |
| THP-NH2 | 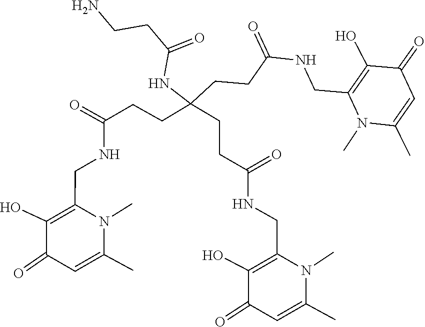 [SMILES: Cc1cc(=O)c(O)c(CNC(=O)CCC(CCC(=O)NCc2c(O)c(=O)cc(C)n2C)(CCC(=O)NCc2c(O)c(=O)cc(C)n2C)NC(=O)CCN)n1C] |
| B6SS | 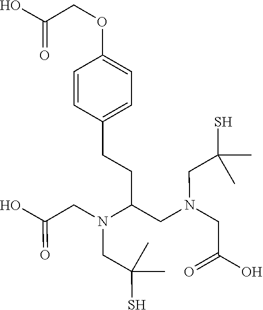 [SMILES: CC(C)(S)CN(CC(=O)O)CC(CCc1ccc(OCC(=O)O)cc1)N(CC(=O)O)CC(C)(C)S] |
| THP- NCS | 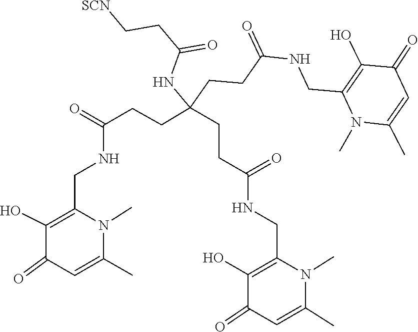 |
| H₂dedpa | 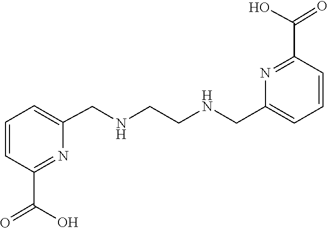 [SMILES: O=C(O)c1cccc(CNCCNCc2cccc(C(=O)O)n2)n1] |
| YM103 | 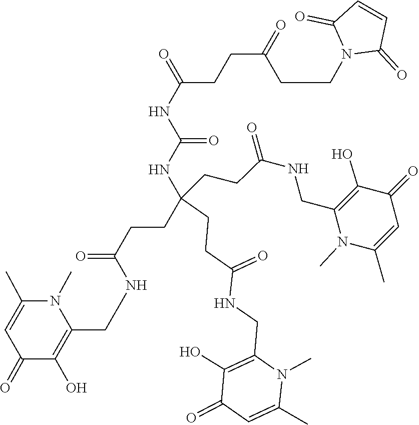 [SMILES: Cc1cc(=O)c(O)c(CNC(=O)CCC(CCC(=O)NCc2c(O)c(=O)cc(C)n2C)(CCC(=O)NCc2c(O)c(=O)cc(C)n2C)NC(=O)NC(=O)CCC(=O)CCN2C(=O)C=CC2=O)n1C] |
| p-SCN- Bn-dedpa | 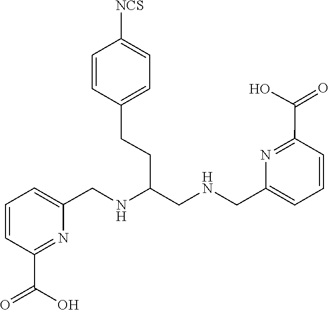 |
| ECC | 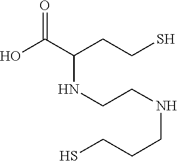 [SMILES: O=C(O)C(CCS)NCCNCCCS] |
| H₄octapa | 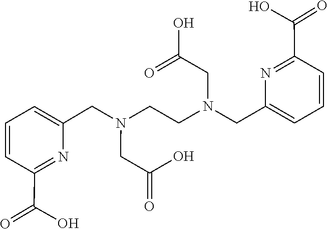 [SMILES: O=C(O)CN(CCN(CC(=O)O)Cc1cccc(C(=O)O)n1)Cc1cccc(C(=O)O)n1] |
| ECD | 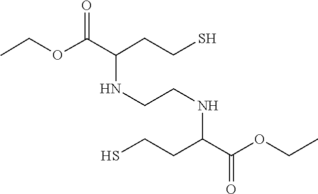 [SMILES: CCOC(=O)C(CCS)NCCNC(CCS)C(=O)OCC] |
| p-SCN- Bn-octapa | 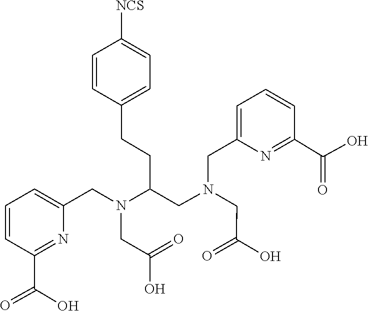 |
| Citric acid | 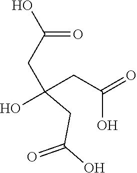 [SMILES: O=C(O)CC(O)(CC(=O)O)CC(=O)O] |
| BFO | 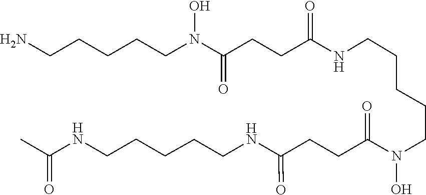 [SMILES: CC(=O)NCCCCCNC(=O)CCC(=O)N(O)CCCCCNC(=O)CCC(=O)N(O)CCCCCN] |
| 6SS | 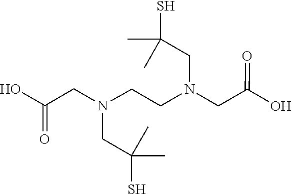 [SMILES: CC(C)(S)CN(CCN(CC(=O)O)CC(C)(C)S)CC(=O)O] |
| RESCA | 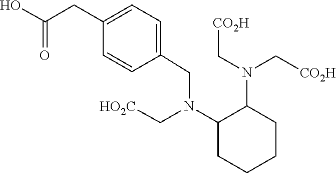 [SMILES: O=C(O)Cc1ccc(CN(CC(=O)O)C2CCCCC2N(CC(=O)O)CC(=O)O)cc1] |
| DFO | 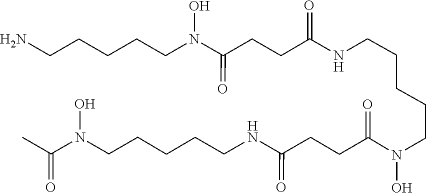 [SMILES: CC(=O)N(O)CCCCCNC(=O)CCC(=O)N(O)CCCCCNC(=O)CCC(=O)N(O)CCCCCN] |
| Pypa | 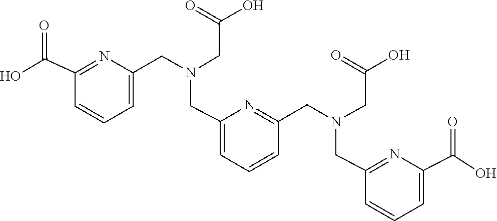 [SMILES: O=C(O)CN(Cc1cccc(CN(CC(=O)O)Cc2cccc(C(=O)O)n2)n1)Cc1cccc(C(=O)O)n1] |
| Py4pa | 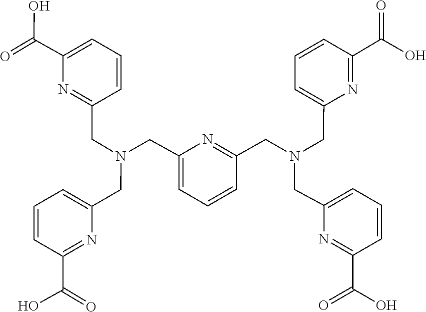 [SMILES: O=C(O)c1cccc(CN(Cc2cccc(CN(Cc3cccc(C(=O)O)n3)Cc3cccc(C(=O)O)n3)n2)Cc2cccc(C(=O)O)n2)n1] |
| H₂CHXhox | 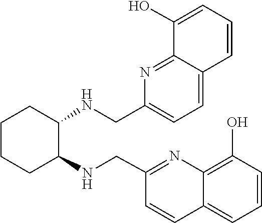 [SMILES: Oc1cccc2ccc(CN[C@H]3CCCC[C@@H]3NCc3ccc4cccc(O)c4n3)nc12] |
| CHXoctapa | 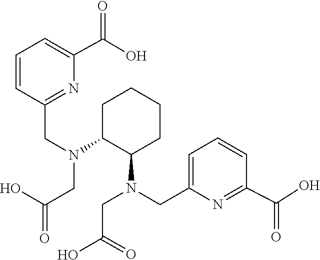 [SMILES: O=C(O)CN(Cc1cccc(C(=O)O)n1)[C@@H]1CCCC[C@H]1N(CC(=O)O)Cc1cccc(C(=O)O)n1] |
| H₂hox | 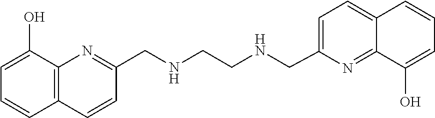 [SMILES: Oc1cccc2ccc(CNCCNCc3ccc4cccc(O)c4n3)nc12] |
| H₄Noneunpa | 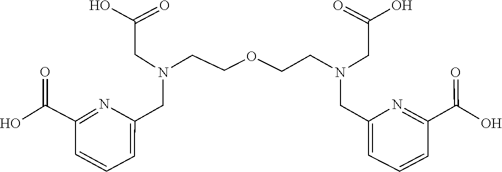 [SMILES: O=C(O)CN(CCOCCN(CC(=O)O)Cc1cccc(C(=O)O)n1)Cc1cccc(C(=O)O)n1] |
| mertiatide | 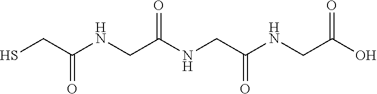 [SMILES: O=C(O)CNC(=O)CNC(=O)CNC(=O)CS] |
| DMSA | 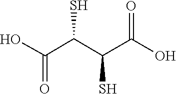 |
| HYNIC | 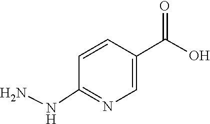 [SMILES: NNc1ccc(C(=O)O)cn1] |
| BOPTA | 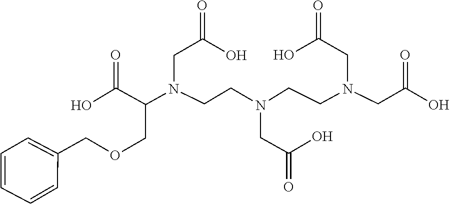 [SMILES: O=C(O)CN(CCN(CC(=O)O)CC(=O)O)CCN(CC(=O)O)C(COCc1ccccc1)C(=O)O] |

Beyond the chelators provided in Tables 3-7, this application covers structurally modified chelators (e.g., containing different or additional substituents, replacement of chelating atoms, etc.) provided that the structurally modified chelators have the same properties as those listed above.

Additional chelators that may be incorporated in FAP-targeting compounds of the present disclosure are described in Holik, et al. “The Chemical Scaffold of Theranostic Radiopharmaceuticals: Radionuclide, Bifunctional Chelator, and Pharmacokinetics Modifying Linker.” Molecules 27.10 (2022): 3062 and Kostelnik, et al. “Radioactive main group and rare earth metals for imaging and therapy.” Chemical reviews 119.2 (2018): 902-956, both of which are incorporated by reference in their entireties.

In some embodiments of the FAP-targeting compounds described herein, the compound comprises at least one chelator (e.g., at least one M), and the chelator is selected from DOTA, DOTAGA, NODAGA, AAZTA, NOTA, and p-SCN-Bn-DOTA. In further embodiments, the chelator is selected from DOTA, DOTAGA, NODAGA, and AAZTA. In still further embodiments, the chelator is selected from DOTA, DOTAGA, and NODAGA. In certain embodiments, the chelator is DOTA. In certain embodiments, the chelator is NODAGA. In certain embodiments, the chelator is DOTAGA.

In some embodiments the chelator or chelator residue includes any additional bridging atom or bridging moiety for attaching the chelator to L¹ or the cyclic peptide P. In a nonlimiting example, bridging atoms or bridging moieties include N, O, S, C(O), or combinations thereof.

The chelator may be covalently attached to L¹ or the cyclic peptide (P) via an attachment at any site on the chelator. For example, a covalent attachment may be formed between a functional group of a functionalized heteroatom, a heteroatom, or an alkyl, aryl, or cycloalkyl group separating the heteroatoms of the chelator to a suitable site on the linker or cyclic peptide.

In an embodiment, when the chelator (e.g., M) is substituted by a carboxy (—COOH) functional group, that functional group can serve as a handle for covalent attachment to L¹ or the cyclic peptide (P). In embodiments, the carboxy-L¹ or carboxy-P attachment point is an amide (—C(O)N(R)—) moiety. In non-limiting embodiments, the language “M is bonded to the —(CH₂CH₂O)r via an amide bond,” covers, for example,

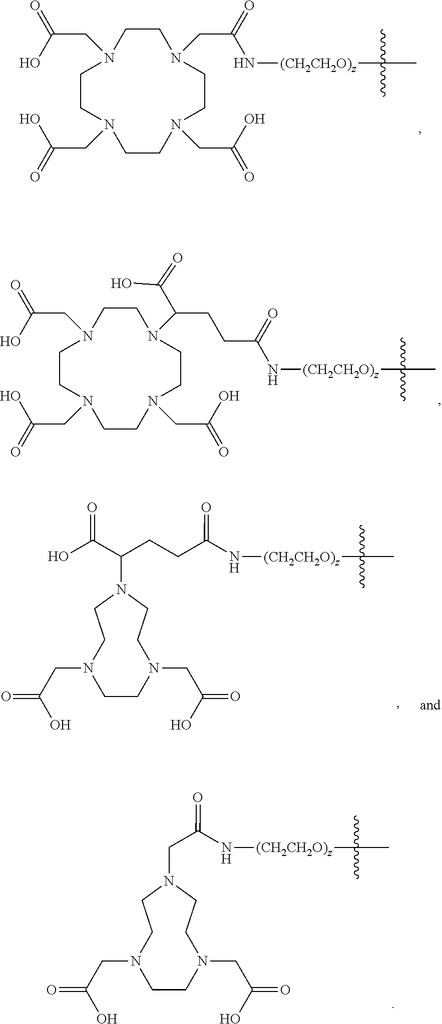

In one or more embodiments of the FAP-targeting compounds described herein (e.g., a compound of formula (I), (Ia), (Ib), (Ic) and (Id), or a pharmaceutically acceptable salt, solvate, stereoisomer, or tautomer thereof), the chelator is attached to L¹, which is connected to a side chain of any amino acid of the cyclic peptide. In certain embodiments, the chelator is attached to L¹, which is connected to a side chain of A⁴. In some embodiments, when the chelator is attached to L¹ and L¹ is connected to a side chain of A⁴, then A⁴, L¹, and M together have a structure selected from:

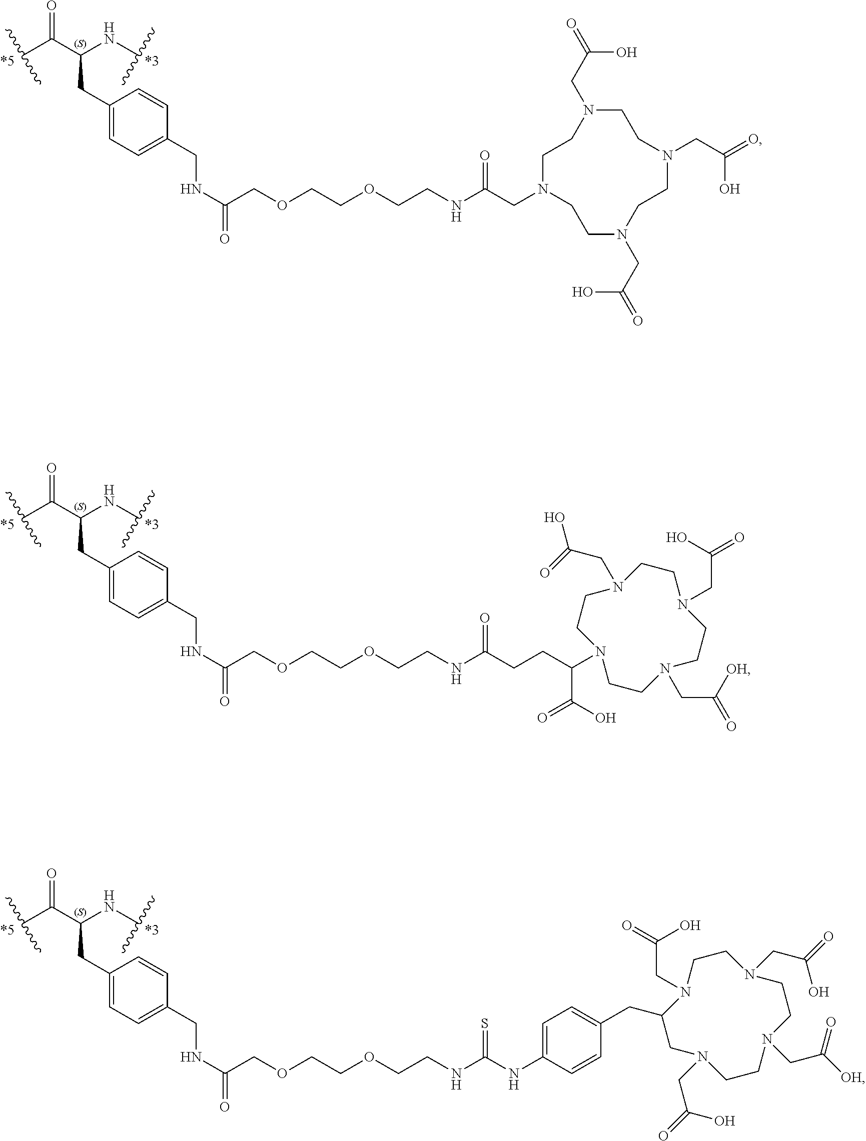

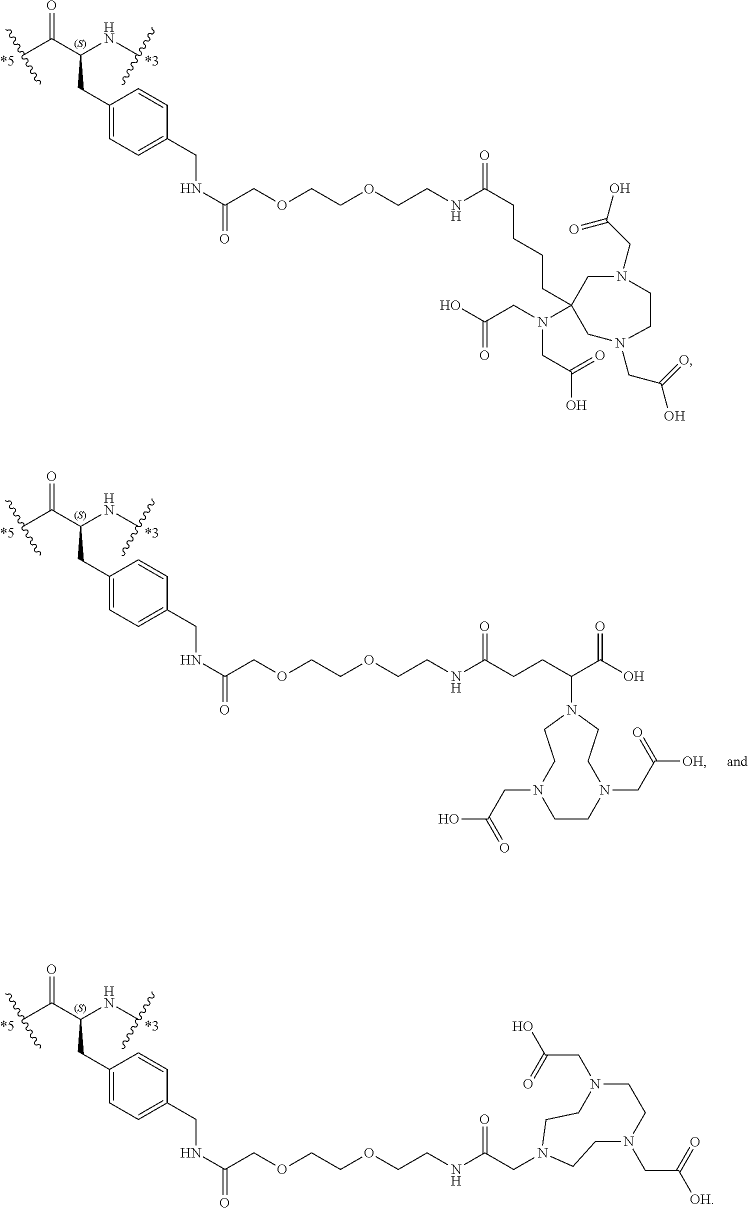

In some embodiments, when the chelator is attached to L¹, and L¹ is connected to a side chain of A⁴ then A⁴, L¹, and M together have a structure selected from:

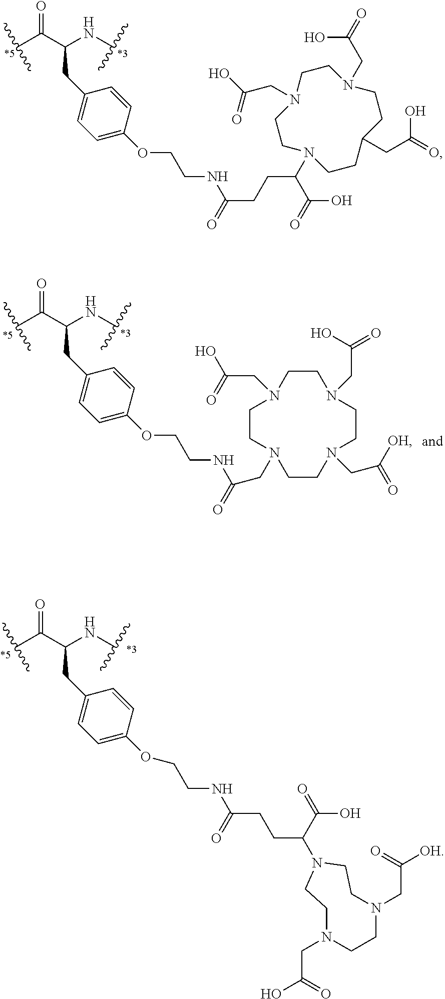 [SMILES: *N[C@@H](Cc1ccc(OCCNC(=O)CCC(C(=O)O)N2CCC(CC(=O)O)CCN(CC(=O)O)CCN(CC(=O)O)CC2)cc1)C(*)=O ; *N[C@@H](Cc1ccc(OCCNC(=O)CN2CCN(CC(=O)O)CCN(CC(=O)O)CCN(CC(=O)O)CC2)cc1)C(*)=O ; *N[C@@H](Cc1ccc(OCCNC(=O)CCC(C(=O)O)N2CCN(CC(=O)O)CCN(CC(=O)O)CC2)cc1)C(*)=O]

In some embodiments, when the chelator is attached to L¹, and L¹ is connected to a side chain of A⁴, then A⁴, L¹, and M together have the structure:

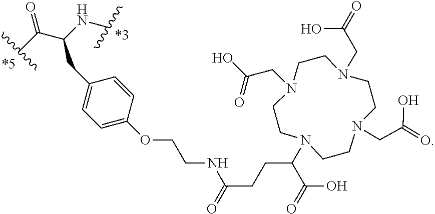 [SMILES: *N[C@@H](Cc1ccc(OCCNC(=O)CCC(C(=O)O)N2CCN(CC(=O)O)CCN(CC(=O)O)CCN(CC(=O)O)CC2)cc1)C(*)=O]

When the chelator is covalently conjugated to L¹ or the cyclic peptide, a chemical substitution or reaction occurs to allow for covalent attachment to the linker or the cyclic peptide. The covalent attachment may be formed via any chemical substitution or reaction known in the art suitable for forming the given attachment. In some embodiments, one or more bonds to a hydrogen atom of the chelator are replaced by a bond to the linker or the cyclic peptide. In some embodiments, a 7c-bond, for example, of a double or triple bond, between two atoms is replaced by a bond from one of the two atoms to the linker or the cyclic peptide, wherein the other of the two atoms includes a new bond, for example, to a hydrogen (such as a reaction of an amine with an isocyanate (—NCS) to yield a thiourea bond or a reaction of maleimide and a thiol to produce a thioether bond). In some embodiments, a carboxylic acid or activated carboxylic acid is reacted with an amine of the cyclic peptide or linker to form an amide bond. Accordingly, one of skill in the art would recognize the various ways in which a chelator can be covalently attached to the linker or the cyclic peptide of any of the formulae presented herein.

### Radionuclides

In some embodiments of the FAP-targeting compounds described herein, M is a radionuclide. In some embodiments of the FAP-targeting compounds described herein, M is chelator radiolabeled with a radionuclide. Accordingly, in various embodiments, the FAP-targeting compounds may be radiolabeled with a diagnostically and/or therapeutically active radionuclide, thus providing a platform for imaging and radiotherapy targeting FAP. As used herein “radionuclide” (which also may be referred to those skilled in the art as radioactive atoms, radioactive elements, radioactive isotopes, or radioisotopes) is any unstable form of a chemical element that releases radiation. In some embodiments, the radionuclide releases or emits P-particles. In some embodiments, the radionuclide releases or emits a-particles. In some embodiments, the radionuclide releases or emits P-particles and a-particles.

The FAP-targeting compounds of the present disclosure may be radiolabeled with a radionuclide at any site of the FAP-targeting peptide. For example, in embodiments in which the FAP-targeting peptide is conjugated directly to a radionuclide, the radionuclide may by covalently attached to the FAP targeting peptide. In other embodiments in which the FAP-targeting peptide is conjugated directly to a radionuclide, such conjugation may rely on ionic interactions, thereby forming a peptide-radionuclide salt. In some embodiments, when the FAP-targeting peptide is conjugated to a chelator, the FAP-targeting peptide may be radiolabeled via chelation of the radionuclide to the chelator. Chelation of a radionuclide to a chelator may be depicted using solid single bonds, dashed single bonds, or a combination thereof. For example, the chelation of ⁶⁸Ga to DOTA can be depicted below with solid single bonds or dashed single bonds. In some embodiments, the charge may also be indicated. For example, when a radionuclide is chelated to a chelating agent, each of the groups chelating the radionuclide may have a negative charge and the radionuclide being chelated may have an opposing positive charge. Such bonds and charges may be depicted herein as follows:

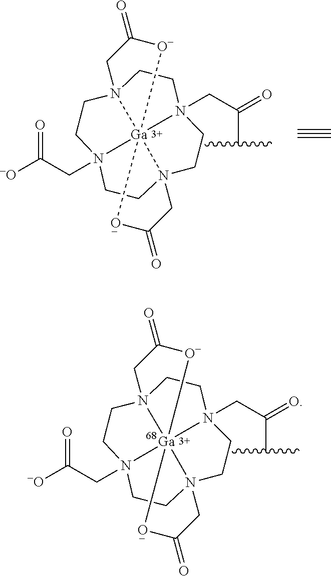

The radionuclide may be a therapeutic radionuclide, diagnostic radionuclide, or both. Suitable radionuclides include, but are not limited to, auger-electron emitting radionuclides, β-emitting (beta or beta-minus-emitting) radionuclides, and α-emitting (alpha-emitting) radionuclides. The selection of the type of radionuclide may depend on the use of the FAP-targeting compound. As will be appreciated by the skilled artisan, several factors may be considered when selecting a radionuclide for use in a FAP-targeting compound, such as, for example, the half-life, the linear energy transfer, the imaging capabilities, and the emission range in tissue. For example, β-emitting radionuclides typically have a longer emission range in tissue (e.g., 1-5 mm) and emit photons in an energy range that is easily imaged, and as such, they may be selected for use in a FAP-targeting compound being used for therapeutic, diagnostic, or theragnostic purposes. On the other hand, α-emitting radionuclides have a shorter emission range in tissue (e.g., 50-100 μm) and a high potency due to the amount of energy deposited per path length traveled (i.e., linear energy transfer), which is approximately 400 times greater than that of electrons. Thus, α-emitting radionuclides may be selected for therapeutic uses in which high potency of the radionuclide is desired.

Accordingly, in some embodiments, the radionuclide is an α-emitting radionuclide. In other embodiments, the radionuclide is a β-emitting radionuclide. In yet other embodiments, the radionuclide is an auger-electron emitting radionuclide.

The radionuclide may be a therapeutic radionuclide, an imaging radionuclide, or both. Accordingly, suitable radionuclides may have a decay energy sufficient for therapeutic and/or diagnostic use. In some embodiments, the decay energy of the radionuclide ranges from about 10 keV to about 6,000 keV. In some embodiments, the decay energy of the radionuclide is between 100 and 1,000 keV. Exemplary radionuclides that may be used to radiolabel the present FAP-targeting compounds are described in Sgouros et al. “Radiopharmaceutical therapy in cancer: clinical advances and challenges.” Nature Reviews Drug Discovery 19.9 (2020): 589-608, which is incorporated herein by reference in its entirety.

In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²T, ¹⁵⁵Th, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th. In some embodiments, the radionuclide is ⁶⁶Ga, ⁶⁷Ga, ⁶⁸Ga, ⁶⁴Cu, ¹⁷⁷Lu, or ²²⁵Ac. In some embodiments, the radionuclide is ¹¹¹In, ^{99m}Tc, ⁶⁸Ga, ⁶⁴Cu, ⁸⁹Zr, ¹²³I, ¹²⁴I, ¹⁸F, ⁹⁰Y, ¹⁷⁷Lu, ¹³¹I, ²²⁵Ac, ²¹¹At, or ²²⁷Th. In certain embodiments, the radionuclide is ¹⁷⁷Lu. In certain embodiments, the radionuclide is ²²⁵Ac. In certain embodiments, the radionuclide is ⁶⁸Ga.

In some embodiments, the radionuclide is ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th.

In some embodiments, the radionuclide is a radiohalogen, e.g., ¹⁸F, ⁷⁵Br, ⁷⁶Br, ⁷⁷Br, ⁸⁰Br, ^{80m}Br, ⁸²Br, ¹²³I, ¹²⁴I, ¹²⁵I, ¹³¹I and ²¹¹At. When the radionuclide is a radiohalogen, the term radiohalogen includes complexes that make the radiohalogen suitable for covalent attachment to the linker or the cyclic peptide or for chelation or complex formation with the chelator. Such complexes contemplated under the term radiohalogen include Si-¹⁸F, B-¹⁸F, and Al-¹⁸F.

In some embodiments, the radiohalogen is connected directly to the cyclic peptide or the linker. For example, ¹³¹I and ¹⁸F (or any other radiohalogen) can be substituted at any position of the linker or the cyclic peptide suitable for substitution with a halo group. In some embodiments, the radiohalogen is ¹⁸F. In some embodiments, when a radiohalogen is connected directly to the cyclic peptide or the linker, the chelator is absent. For example, when the radiohalogen is ¹⁸F, it may be attached to L¹ via a prosthetic group and L¹ is a linker attached to a side chain of an amino acid of the cyclic peptide (P). In further embodiments, M (when ¹⁸F) and L together have the structure:

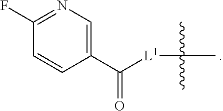

Alternatively, in some embodiments, the radiohalogen forms a complex with the chelator. For example, a radiohalogen complex such as, for example, Si—¹⁸F, B—¹⁸F, and Al—¹⁸F may chelate to a chelator of the presently described FAP-targeting compounds. One of ordinary skill in the art would understand that various radiohalide ions and metal or nonmetal elements may be combined to form a radiohalogen complex. Any such radiohalogen complex may be chelated to a chelator of the FAP-targeting compounds. In some embodiments, Si—¹⁸F is chelated to a chelator. In some embodiments, B—¹⁸F is chelated to a chelator. In some embodiments, Al—¹⁸F is chelated to a chelator. The FAP-targeting compound of the present disclosure may be radiolabeled with one radionuclide or to more than one radionuclide. In some embodiments, the FAP-targeting compound is radiolabeled with one radionuclide. In some embodiments, the FAP-targeting compound is radiolabeled with more than one radionuclide. For example, the FAP-targeting compound made be radiolabeled with one radionuclide via complexation to the chelator and to another radionuclide via a direct covalent attachment to the linker, cyclic peptide, or the chelator. In some embodiments, a first radionuclide is chelated to the chelator and a second radionuclide is covalently attached to the cyclic peptide. In other embodiments, a first radionuclide is chelated to the chelator and a second radionuclide is covalently attached to the linker. In yet other embodiments, a first radionuclide is chelated to the chelator and a second radionuclide is covalently attached to the chelator.

In embodiments in which the FAP-targeting compound is radiolabeled with more than one radionuclide, each of the radionuclides may be the same or different. In some embodiments, two of the same radionuclides are used to radiolabel the FAP-targeting compound of the present disclosure. In some embodiments, two different radionuclides are used to radiolabel the FAP-targeting compound of the present disclosure. In some embodiments, the FAP-targeting compound is radiolabeled with a diagnostic radionuclide and a therapeutic radionuclide. In some embodiments, the FAP-targeting compound is radiolabeled with a radiohalogen and a radionuclide other than a radiohalogen. In some embodiments, the FAP-targeting compound is radiolabeled with a first radiohalogen and a second radiohalogen, where the first radiohalogen is different from the second radiohalogen.

In some embodiments, the radionuclide is a therapeutically active radionuclide. Suitable therapeutically active radionuclides include, but are not limited to, ⁶⁷Cu, ¹⁸⁶Re, ¹⁸⁸Re, ⁹⁰Y ¹⁷⁷Lu, ¹⁶¹Tb, ¹⁵³Sm ²¹³Bi, ¹³¹I, ¹⁴⁹Tb, ⁴⁷Sc, ²²⁵Ac, ²¹²Pb, ²¹¹At, ²²³Ra, ²²⁷Th, and ²²⁶Th. In some embodiments, the radionuclide is a therapeutically active radionuclide selected from ⁶⁷Cu, ¹⁸⁸Re, ⁹⁰Y ¹⁷⁷Lu, ²¹³Bi, ¹³¹I, ⁴⁷Sc, ²²⁵Ac, ²¹²Pb, ²¹¹At, and ²²⁷Th. In particular embodiments, the radionuclide is a therapeutically active radionuclide selected from ⁹⁰Y, ¹⁷⁷Lu, ¹³¹I, ²²⁵Ac, ²¹¹At, and ²²⁷Th. In a particular embodiment, the therapeutically active radionuclide is ¹⁷⁷Lu.

Alternatively, in some embodiments, the radionuclide is a diagnostically active radionuclide. Suitable diagnostically active radionuclides include, but are not limited to, ¹¹¹In, ^{99m}Tc, ^{94m}Tc, ⁶⁷Ga, ⁶⁸Ga ²⁰³Pb ⁶⁴Cu, ⁸⁶Y, ⁸⁹Zr, ⁵¹Mn, ⁵²Mn, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, and ²⁰¹Tl. In some embodiments, the radionuclide is a diagnostically active radionuclide selected from ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Th, ¹⁵⁵Th ⁴⁴Sc, and ⁴³Sc. In particular embodiments, the radionuclide is a diagnostically active radionuclide selected from ¹¹¹In, ^{99m}Tc ⁶⁸Ga, ⁶⁴Cu, ⁸⁹Zr, ¹²³I, ¹²⁴I, and ¹⁸F. In a particular embodiment, the diagnostically active radionuclide is ⁶⁸Ga. In another particular embodiment, the diagnostically active radionuclide is ¹⁸F. In another particular embodiment, the diagnostically active radionuclide is ⁶⁴Cu.

In some embodiments, the radionuclide coordinated to or covalently attached to the FAP-targeting compound described herein is stable in vivo. Stable radionuclides may have a half-life that allows for therapeutic and/or diagnostic medical use. For example, the radionuclide may have a half-life from about 10 minutes to about 50 days. In some embodiments, the radionuclide has a half-life between 1 hour and 20 days. In some embodiments, the radionuclide has a half-life between 1 day and 10 days.

In some embodiments, the radionuclide is detectable by positron emission spectroscopy (PET), positron emission tomography and computerized tomography (PET/CT), or single photon emission computed tomography (SPECT).

In some embodiments, the radionuclide is covalently attached directly to the cyclic peptide or the linker of the FAP-targeting compound. In some embodiments, the radionuclide is coordinated to the chelating ligand of the FAP-targeting compound. Various radionuclides may be complexed to various chelators disclosed herein. One of ordinary skill in the art would recognize the appropriate selection of chelator and radionuclide for any use or method contemplated by the instant disclosure.

### Additional Embodiments of Formulae (I), (Ia), (Ib), (Ic) and (Id)

As previously described, the FAP-targeting compounds of the present disclosure may be compounds of formula (I), (Ia), (Ib), (Ic) or (Id):

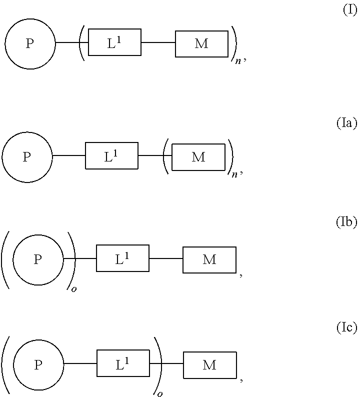

or pharmaceutically acceptable salts, solvates, stereoisomers, and tautomers thereof, wherein P, L¹ and M are as previously described and n and o are each independently 1, 2, 3, or 4.

It is conceived herein that the FAP-targeting compound may include 1, 2, 3, or 4 P, P-L¹ moieties, M, or M-L¹ moieties. In some embodiments, the FAP-targeting compounds includes 1, 2, 3, or 4 cyclic peptides, 1, 2, or 3, cyclic peptides, or 1 or 2 cyclic peptides. In some embodiments, the FAP-targeting compounds includes 1, 2, 3, or 4 P-L¹ moieties, 1, 2, or 3, P-L¹ moieties, or 1 or 2 P-L¹ moieties. In some embodiments, the FAP-targeting compounds includes 1, 2, 3, or 4 M moieties, 1, 2, or 3, M moieties, or 1 or 2 M moieties. In some embodiments, the FAP-targeting compounds includes 1, 2, 3, or 4 m-L¹ moieties, 1, 2, or 3, M-L¹ moieties, or 1 or 2 M-L¹ moieties.

In embodiments in which the FAP-targeting peptide includes at least two cyclic peptides, each cyclic peptide can be the same or different. In embodiments in which the FAP-targeting peptide includes at least two P-L¹ moieties, each cyclic peptide and each L¹ may be independently selected and can be the same or different. In embodiments in which the FAP-targeting peptide includes at least two M moieties, each M moiety can be the same or different. In embodiments in which the FAP-targeting peptide includes at least two M-L¹ moieties, each M moiety and each L¹ may be independently selected and can be the same or different.

In some embodiments, the compounds of formulae (I), (Ia), (Ib), and (Ic) are compounds of formulae (I-i), (Ia-i), (Ib-i) and (Ic-i):

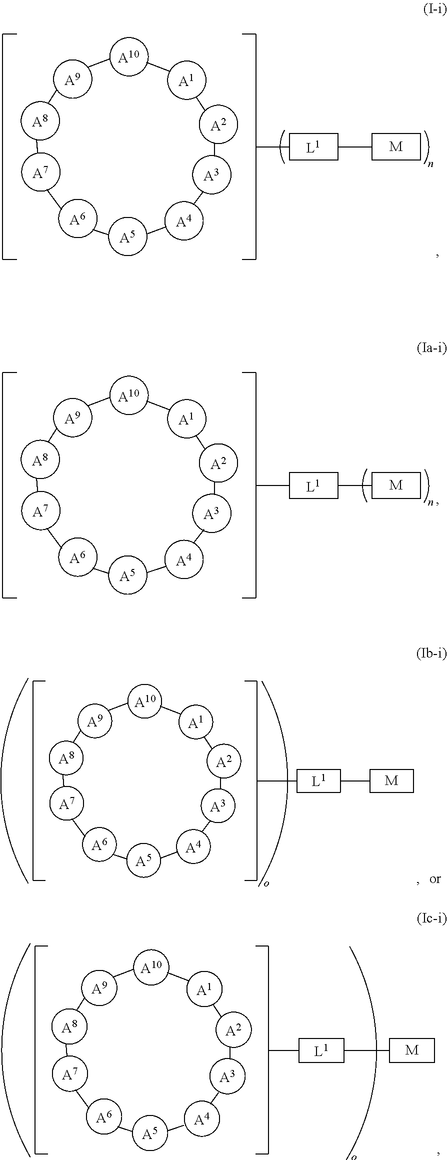

wherein A¹-A¹⁰ are as defined elsewhere herein,
L¹ is, independently at each occurrence, selected from the group consisting of a bond and a linker;
M is, independently at each occurrence, selected from the group consisting of an imaging agent, a chelating agent, and a radionuclide the chelating agent is optionally radiolabeled with a radionuclide;
n is 1, 2, 3, or 4;
o is 1, 2, 3, or 4;
and wherein any of A¹-A¹⁰ and L¹ is optionally substituted with an albumin binder.

In certain embodiments, the FAP-targeting compound is compound of formula (I):

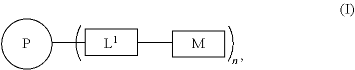

or a pharmaceutically acceptable salt, solvate, stereoisomer, or tautomer thereof, wherein n is 1. For example, in some embodiments, the compound of formula (I) is a compound of formula (Id):

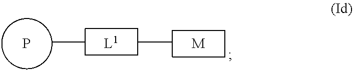

or a pharmaceutically acceptable salt, solvate, stereoisomer, or tautomer thereof,
wherein:
P is a cyclic (FAP-targeting) peptide;
L¹ is a bond or linker; and
M is a chelating agent optionally radiolabeled with a radionuclide;
and wherein the cyclic peptide, linker, chelating agent, and radionuclide are as disclosed elsewhere herein.

In some embodiments, the compound of formula (Ib) is a compound of formula (ib-ii):

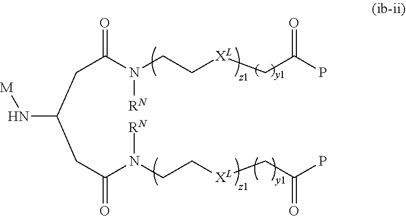

or a pharmaceutically acceptable salt, solvate, stereoisomer, or tautomer thereof,
wherein:
X^{L} is CH₂, N, or O;
R^{N} is H, C₁₋₃-alkyl, or acyl;
y1, independently, at each occurrence, is 0, 1, 2, 3, 4, 5, or 6;
z1, independently at each occurrence, is 0, 1, 2, 3, or 4;
P is a cyclic (FAP-targeting) peptide; and
M is a chelating agent optionally radiolabeled with a radionuclide; and wherein the cyclic peptide, chelating agent, and radionuclide are as disclosed elsewhere herein.

In an embodiment of formula (ib-ii), the compound is a compound of formula (ib-iii):

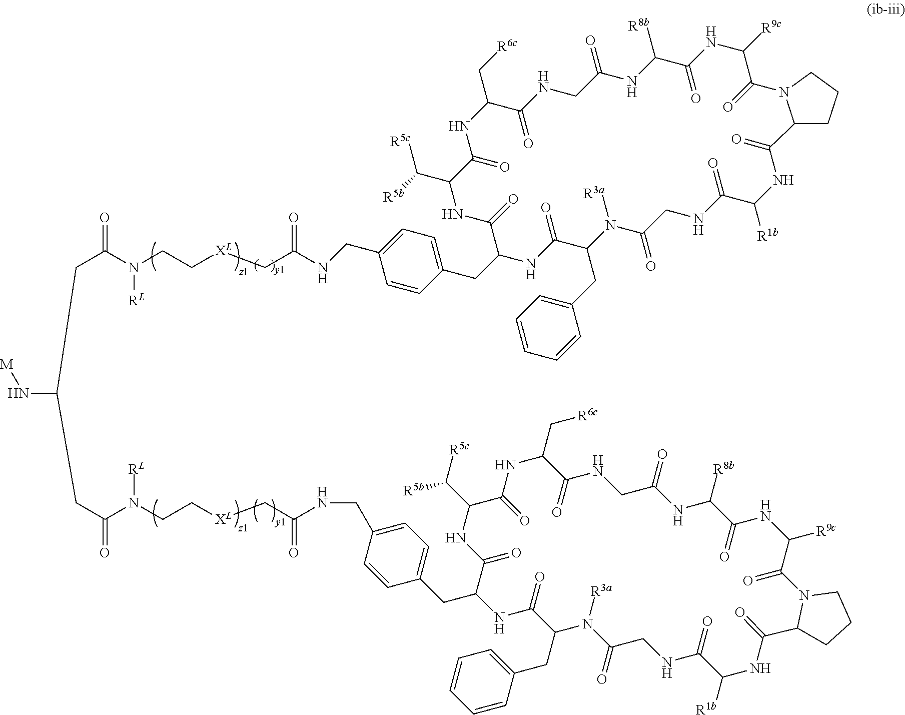

or a pharmaceutically acceptable salt, solvate, stereoisomer, or tautomer thereof,
wherein:
X^{L} is CH₂, N, or O;
R^{N} is H, C₁₋₃-alkyl, or acyl;
y1, independently, at each occurrence, is 0, 1, 2, 3, 4, 5, or 6;
z1, independently at each occurrence, is 0, 1, 2, 3, or 4;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{1c};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH;
R^{3a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, and —C(O)R^{5d};
R^{d} is 4-7 membered heterocycloalkyl optionally substituted with 1, 2, 3, or 4 groups independently selected from C₁₋₃-alkyl, —CN, halo, and —OH;
R^{6c} is a 5 to 10 membered heteroaryl optionally substituted with 1, 2, 3, or 4 groups independently selected from C₁₋₃-alkyl, —CN, halo, —OH, and —OC₁₋₆-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆alkyl-O—C(O)R^{8c}, and —C₁₋₆alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH;
R^{9c} is C₁₋₆-alkyl optionally substituted with 1, 2, 3, or 4 groups independently selected from —CN, —C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
M is selected from DOTA, DOTAGA, NODAGA, AAZTA, NOTA, and p-SCN-Bn-DOTA, and wherein M is optionally radiolabeled with a radionuclide.

In some embodiments of the compound of formulae (Ib), (Ib-i), (Ib-ii), and (Ib-iii), or a pharmaceutically acceptable salt, solvate, stereoisomer, or tautomer thereof, is a compound selected from Examples M1-M5.

In some embodiments of the compound of formula (Id), or a pharmaceutically acceptable salt, solvate, stereoisomer, or tautomer thereof, M is a chelator optionally radiolabeled with a radionuclide, wherein the chelator is selected from DOTA, DOTAGA, NODAGA, AAZTA, NOTA, and p-SCN-Bn-DOTA. In some embodiments of the compound of formula (Id), or a pharmaceutically acceptable salt, solvate, stereoisomer, or tautomer thereof, M is a chelator optionally radiolabeled with a radionuclide, wherein the chelator is selected from DOTA, DOTAGA, and NODAGA. In some embodiments of the compound of formula (Id), or a pharmaceutically acceptable salt, solvate, stereoisomer, or tautomer thereof, M is a chelator optionally radiolabeled with a radionuclide, wherein the chelator is DOTA.

In some embodiments of the compound of formula (Id), or a pharmaceutically acceptable salt, solvate, stereoisomer, or tautomer thereof, M is a chelator that is not radiolabeled with a radionuclide. Alternatively, in some embodiments of the compound of formula (Id), or a pharmaceutically acceptable salt, solvate, stereoisomer, or tautomer thereof, M is a chelator that is radiolabeled with a radionuclide.

In some embodiments, the compound of formula (Id) is a compound of formula (Id-i):

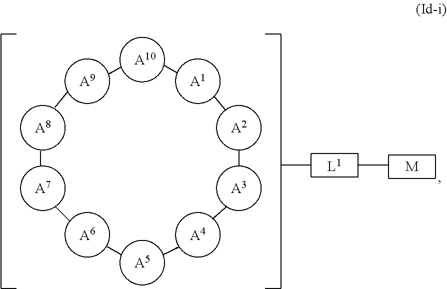

or a pharmaceutically acceptable salt, solvate, stereoisomer, or tautomer thereof,
wherein A¹-A¹⁰ are as defined elsewhere herein, and L¹ is connected to the cyclic FAP-targeting peptide via a side chain of any one of A¹-A¹⁰.

In some embodiments, the compound of formula (Id), or a pharmaceutically acceptable salt, solvate, stereoisomer, or tautomer thereof, is a compound of formula (Id-ii):

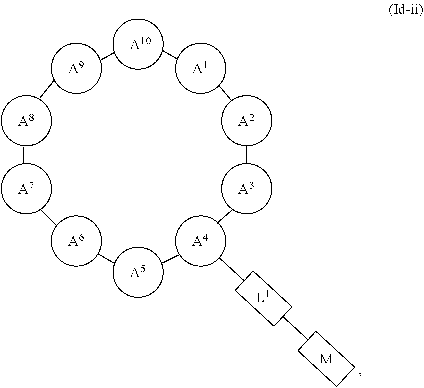

or a pharmaceutically acceptable salt, solvate, stereoisomer, or tautomer thereof,
wherein L¹ is connected to the cyclic FAP-targeting peptide via a side chain of A⁴. In some embodiments of the compound of formula (Id-ii), or a pharmaceutically acceptable salt, solvate, stereoisomer, or tautomer thereof, L¹ is connected to the cyclic FAP-targeting peptide via a side chain NH₂ of A⁴.

In some embodiments, the compound of formula (Id) (and subgenera of formula (Id), e.g., formulae (Id-i) and (Id-ii)), or a pharmaceutically acceptable salt, solvate, stereoisomer, or tautomer thereof, has the following definitions:

A¹ is

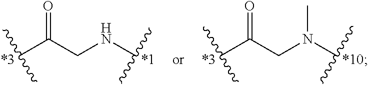

A² is

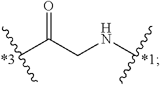 [SMILES: *NCC(*)=O]

A³ is

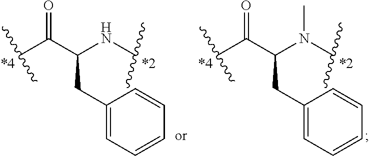 [SMILES: *N[C@@H](Cc1ccccc1)C(*)=O ; *C(=O)[C@H](Cc1ccccc1)N(*)C]

A⁵ is

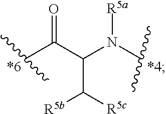

wherein
R^{5a} is H or CH₃;
R^{5b} is selected from the group consisting of H, and —OH,
R^{5c} is selected from the group consisting of H and C₁₋₈-alkyl, wherein the C₁₋₈-alkyl of R^{5c} is optionally substituted with —OH, —OC₁₋₆-alkyl, and —C(O)R^{5d},
R^{5d} is 5 membered heterocycloalkyl optionally substituted with 1, 2, or 3 groups independently selected from —CN, halo, and —OH;
A⁶ is

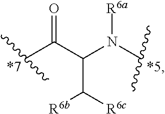

wherein
R^{6a} is H or CH₃;
R^{6b} is selected from the group consisting of H and C₁₋₃-alkyl,
R^{6c} is an 8 to 10 membered heteroaryl optionally substituted with a group selected from C₁₋₃-alkyl, halo, —OH, and —OC₁₋₃-alkyl

A⁷ is

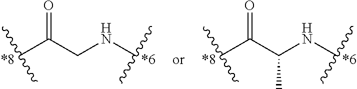

A⁸ is

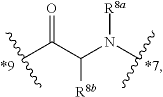

wherein
R^{8a} is selected from the group consisting of H, C₁₋₃-alkyl, and —CH₂-phenyl, wherein the C₁₋₃-alkyl and —CH₂-phenyl of R^{8a} are each optionally substituted with 1, 2, or 3 substituents independently selected from —NH₂, —NHC₁₋₆-alkyl, —N(C₁₋₆-alkyl)₂, —OH, —OC₁₋₃-alkyl, and C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆-alkyl-O—C(O)R^{8c}, and —C₁₋₆-alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH;
A⁹ is

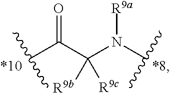

wherein
R^{9a} is H or CH₃;
R^{9b} is H or CH₃;
R^{9c} is C₁₋₆-alkyl optionally substituted with a group selected from —C(O)OH, —C(O)NH₂, —NH₂, —NHC₁₋₆-alkyl, —N(C₁₋₆-alkyl)₂, and —OH;
A¹⁰ is a moiety of formula (IIA):

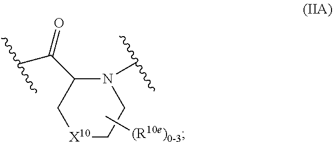

wherein:
X¹⁰ is absent or selected from the group consisting of O, N, or CHR^{10e}; and
each R^{1e} is independently selected from —OH, —NH₂, —C₁₋₃-alkyl, halo, and —C(O)R^{10d}, wherein R^{10d} is selected from the group consisting of H, —OH, and C₁₋₆-alkyl and —OC₁₋₆-alkyl;
A⁴ is bound to linker L¹, wherein A⁴ and L¹ together have a structure selected from:

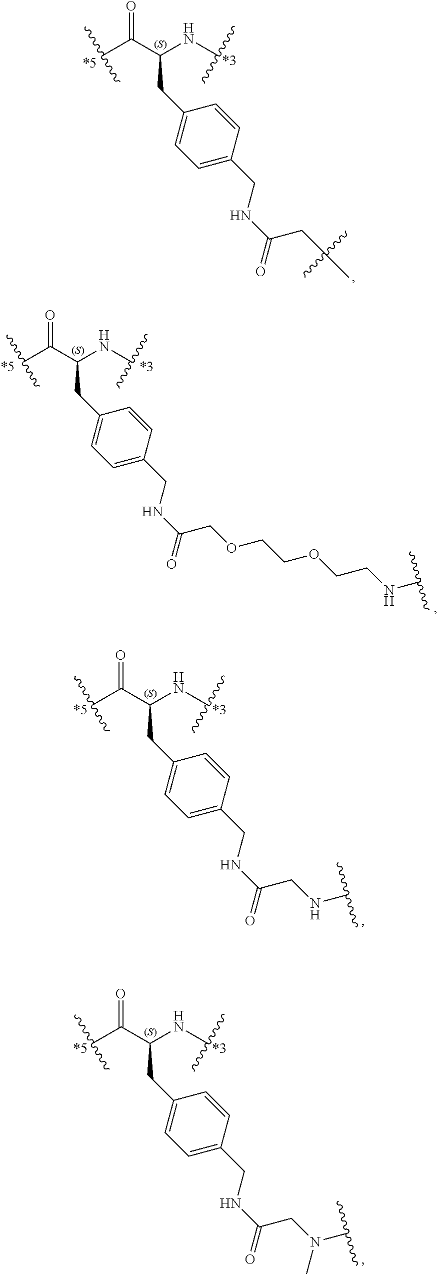

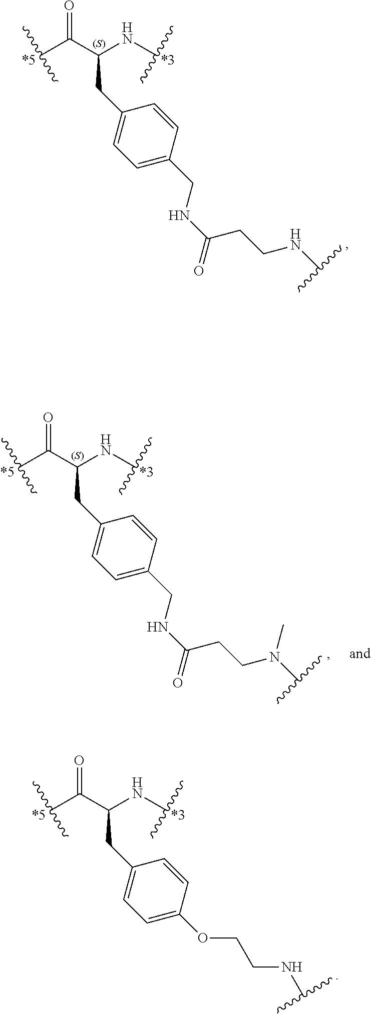

wherein L¹ is bound to M, and M is a chelator optionally radiolabeled with a radionuclide. In an embodiment, M is selected from DOTA, DOTAGA, NODAGA, AAZTA, NOTA, and p-SCN-Bn-DOTA, wherein M is bonded to L¹ via an amide bond. In another embodiment, M is DOTA, wherein M is bonded to L¹ via an amide bond.

In an embodiment, the compound of formula (Id), or a pharmaceutically acceptable salt, solvate, stereoisomer, or tautomer thereof, is a compound of formula (Id-iia):

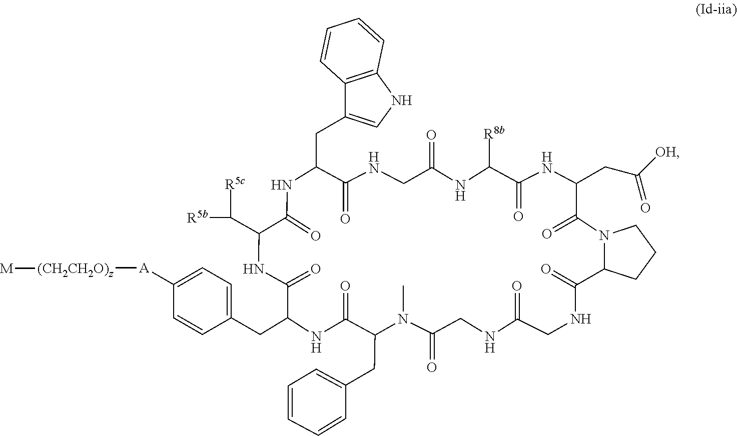

or a pharmaceutically acceptable salt, solvate, or tautomer thereof, wherein:
M is a chelator optionally radiolabeled with a radionuclide, wherein M is bonded to the —(CH₂CH₂O)_{z}— via an amide bond;
z is 1, 2, 3, or 4;
A is absent, —CH₂—C(O)—N(H)—CH₂—, or —CH₂—N(H)—C(O)—CH₂—;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, or —C(O)R^{1d};
R^{5d} is 4-7 membered heterocycloalkyl optionally substituted with 1, 2, 3, or 4 groups independently selected from C₁₋₃-alkyl, —CN, halo, and —OH;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆alkyl-O—C(O)R^{8c}, and —C₁₋₆alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH.

In an embodiment of formula (Id-iia), or a pharmaceutically acceptable salt, solvate, or tautomer thereof,
M is selected from DOTA, DOTAGA, NODAGA, AAZTA, NOTA, and p-SCN-Bn-DOTA, wherein M is bonded to the —(CH₂CH₂O_{z})— via an amide bond;
z is 1 or 2;
A is absent, —CH₂—C(O)—N(H)—CH₂—, or —CH₂—N(H)—C(O)—CH₂—;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with —OC₁₋₆-alkyl or —C(O)R^{5d};
R^{5d} is 5-membered heterocycloalkyl optionally substituted with 1, 2, or 3 groups independently selected from —CN, halo, and —OH;
R^{8b} is selected from the group consisting of H, C₁₋₆-alkyl, —C₁₋₆-alkyl-phenyl, and —C₁₋₆-alkyl-NH—C(O)R^{8c}, wherein the —C₁₋₆-alkyl-phenyl of R^{8b} is optionally substituted with halo, —NH₂, or —OH; and
R^{8c} is a 6-membered heterocycloalkyl optionally substituted with —C₁₋₆-alkylC(O)OH or —C(O)OH.

In an embodiment of formula (Id), the compound of formula (Id), or a pharmaceutically acceptable salt, solvate, stereoisomer, or tautomer thereof, is a compound of formula (Id-iib):

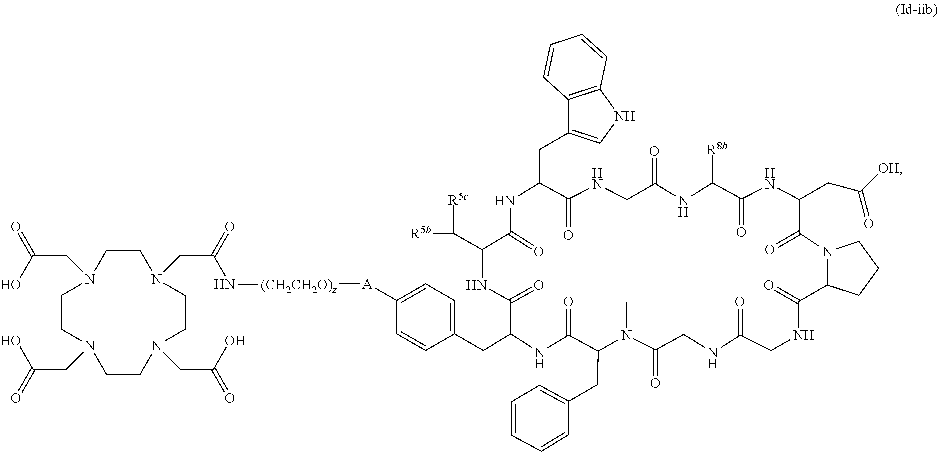

or a pharmaceutically acceptable salt, solvate, or tautomer thereof, which is optionally radiolabeled with a radionuclide, wherein:
z is 1, 2, 3, or 4;
A is absent, —CH₂—C(O)—N(H)—CH₂—, or —CH₂—N(H)—C(O)—CH₂—
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, or —C(O)R^{1d};
R^{5d} is 4-7 membered heterocycloalkyl optionally substituted with 1, 2, 3, or 4 groups independently selected from C₁₋₃-alkyl, —CN, halo, and —OH;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆alkyl-O—C(O)R^{8′}, and —C₁₋₆alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH.

In an embodiment of formula (Id-iib), or a pharmaceutically acceptable salt, solvate, or tautomer thereof,
z is 1 or 2;
A is absent, —CH₂—C(O)—N(H)—CH₂—, or —CH₂—N(H)—C(O)—CH₂—;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with —OC₁₋₆-alkyl or —C(O)R^{1d};
R^{8d} is 5-membered heterocycloalkyl optionally substituted with 1, 2, or 3 groups independently selected from —CN, halo, and —OH;
R^{8b} is selected from the group consisting of H, C₁₋₆-alkyl, —C₁₋₆-alkyl-phenyl, and —C₁₋₆-alkyl-NH—C(O)R^{8c}, wherein the —C₁₋₆-alkyl-phenyl of R^{b} is optionally substituted with halo, —NH₂, or —OH; and
R^{8c} is a 6-membered heterocycloalkyl optionally substituted with —C₁₋₆-alkylC(O)OH or —C(O)OH.

In an embodiment of formula (Id), the compound of formula (Id), or a pharmaceutically acceptable salt, solvate, stereoisomer, or tautomer thereof, is selected from the group consisting of:

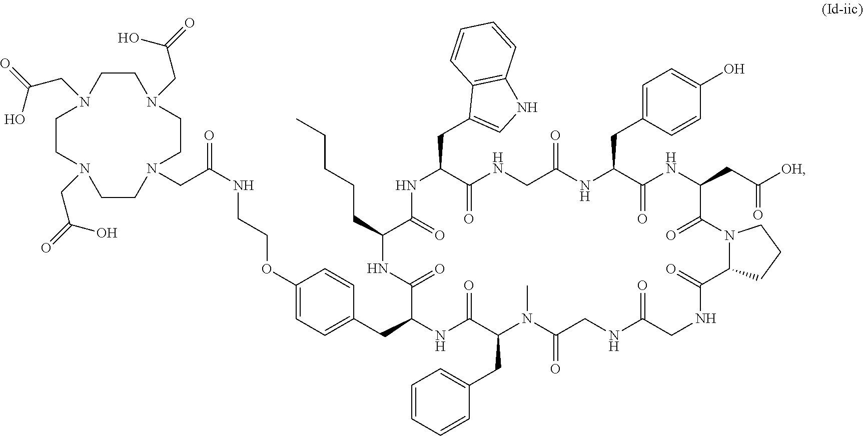 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O]

wherein the compound of formula (Id-iic) is optionally radiolabeled with a radionuclide,

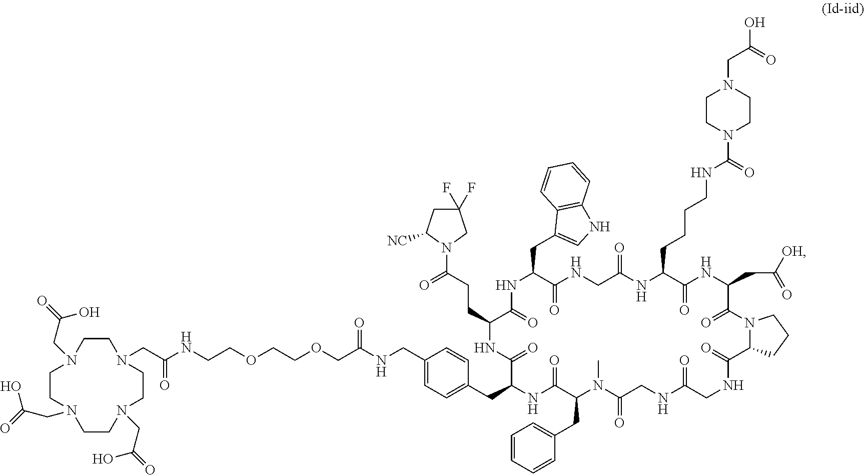 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@@H]1Cc1ccccc1]

wherein the compound of formula (Id-iid) is optionally radiolabeled with a radionuclide,

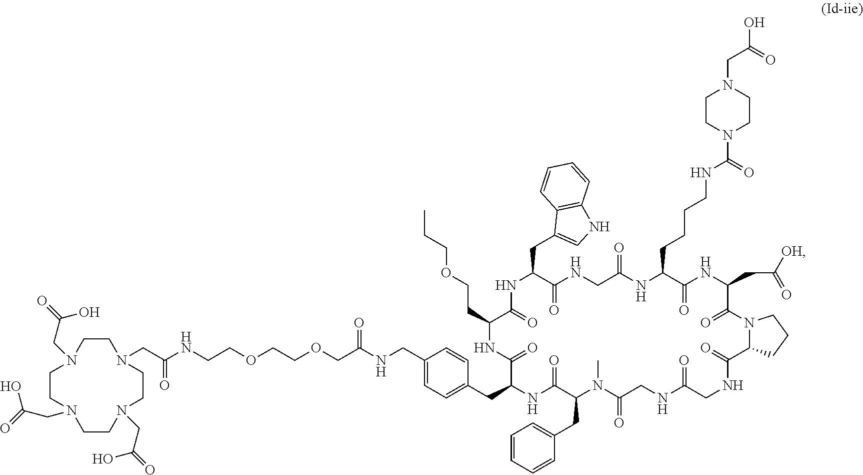 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iie) is optionally radiolabeled with a radionuclide, and

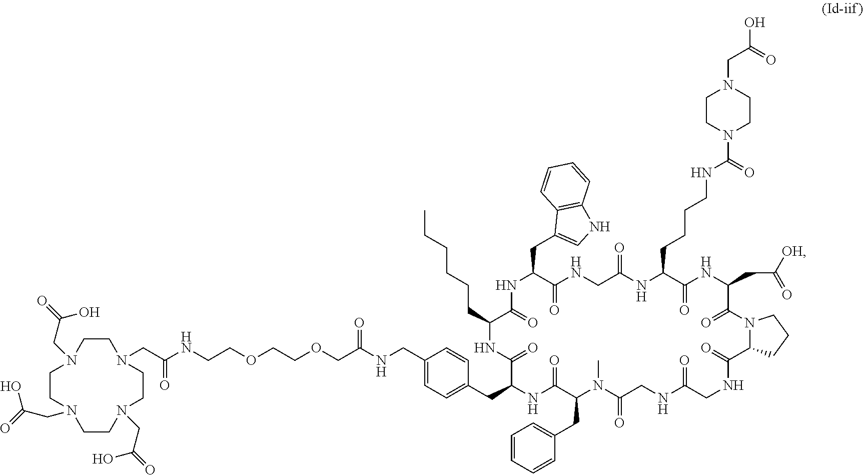 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iif) is optionally radiolabeled with a radionuclide.

In an embodiment of formula (Id), the compound of formula (Id), or a pharmaceutically acceptable salt, solvate, stereoisomer, or tautomer thereof, is selected from the group consisting of:

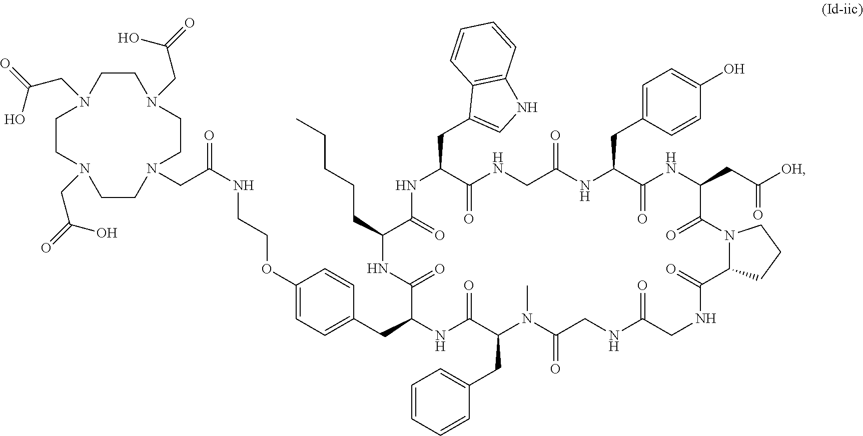 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O]

wherein the compound of formula (Id-iic) is radiolabeled with a radionuclide,

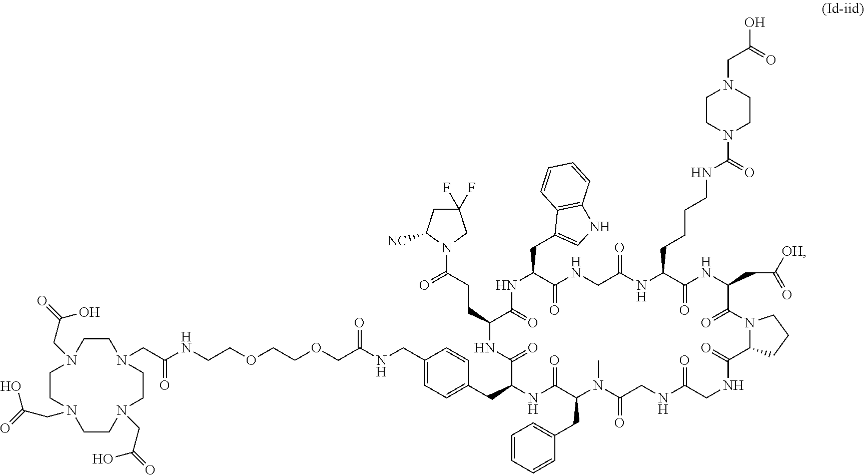 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@@H]1Cc1ccccc1]

wherein the compound of formula (Id-iid) is radiolabeled with a radionuclide,

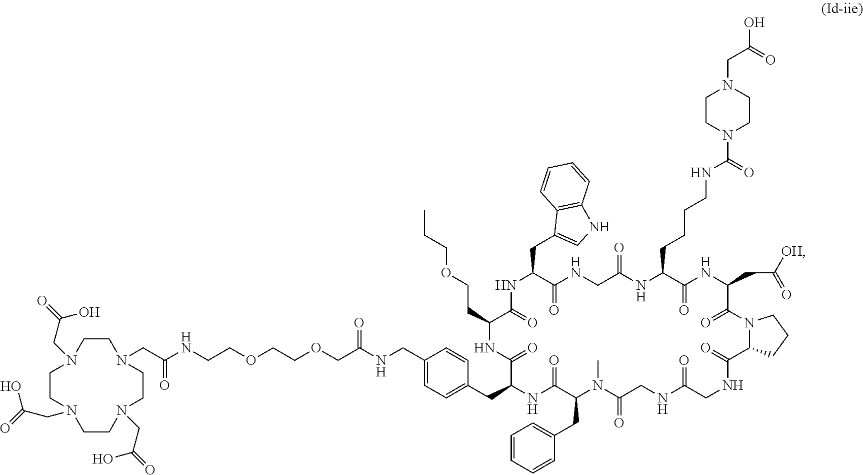 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iie) is radiolabeled with a radionuclide, and

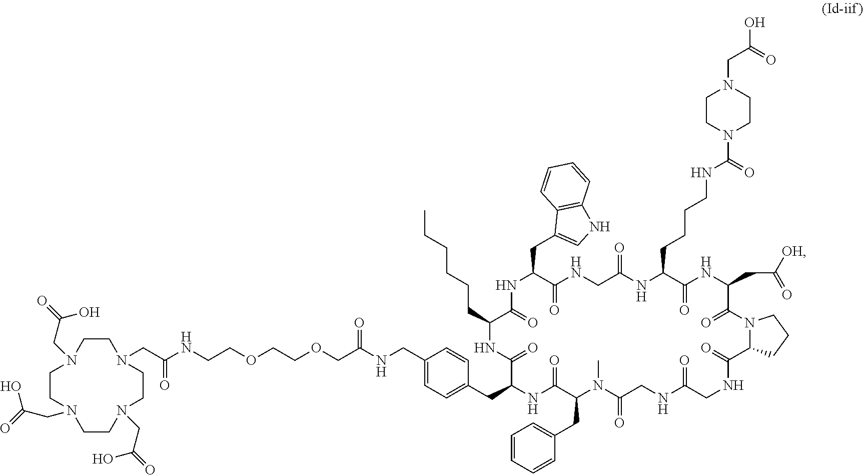 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iif) is radiolabeled with a radionuclide.

In an embodiment of formula (Id), the compound of formula (Id), or a pharmaceutically acceptable salt, solvate, stereoisomer, or tautomer thereof, is selected from the group consisting of:

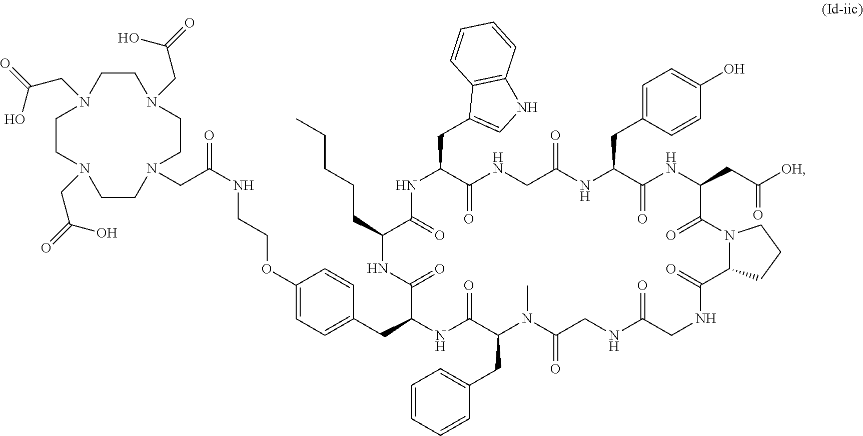 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O]

wherein the compound of formula (Id-iic) is radiolabeled with ¹⁷⁷Lu,

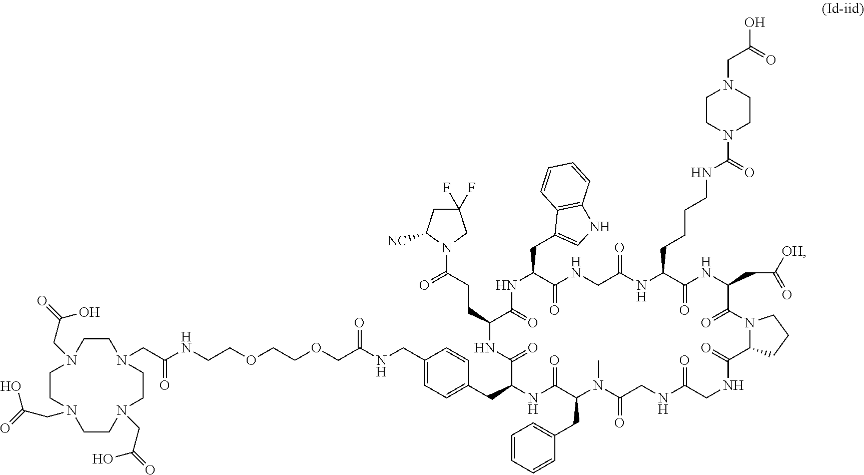 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@@H]1Cc1ccccc1]

wherein the compound of formula (Id-iid) is radiolabeled with ¹⁷⁷Lu,

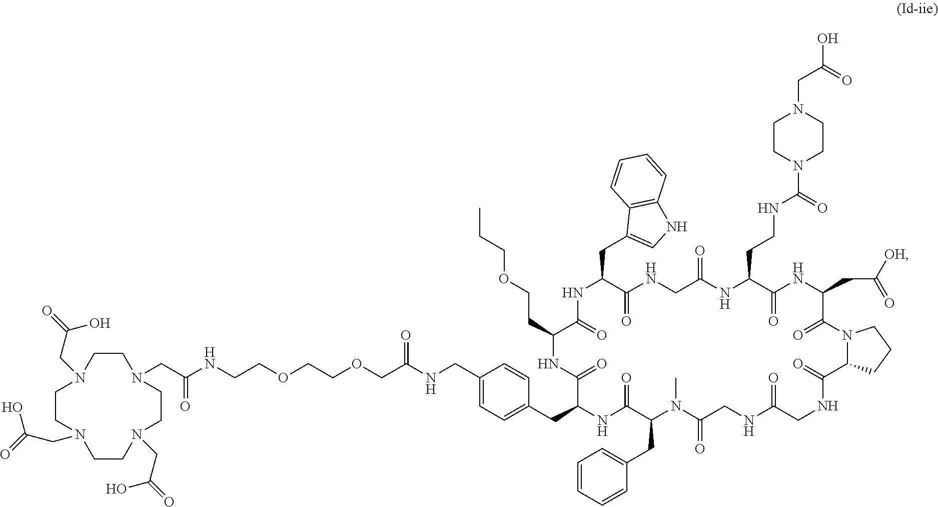 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCNC(=O)N2CCN(CC(=O)O)CC2)NC(=O)CNC(=O)[C@H](Cc2c[nH]c3ccccc23)NC1=O]

wherein the compound of formula (Id-iie) is radiolabeled with ¹⁷⁷Lu, and

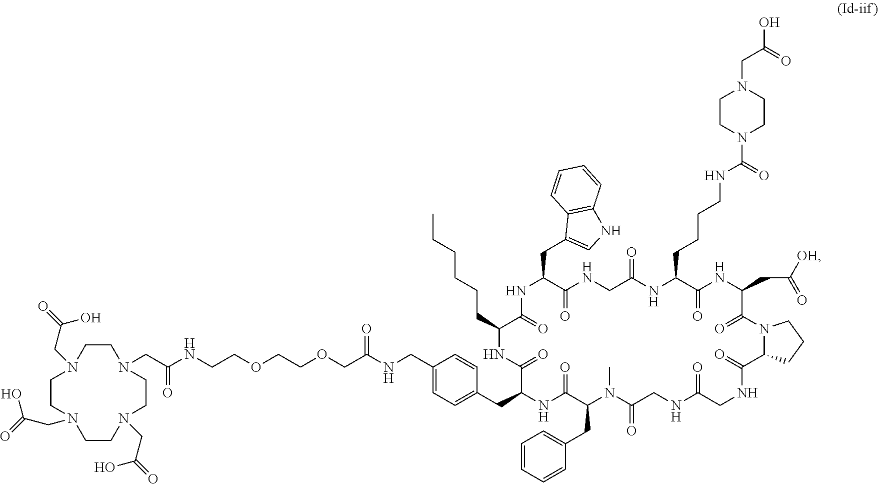 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iif) is radiolabeled with ¹⁷⁷Lu.

In an embodiment of formula (Id), the compound of formula (Id), or a pharmaceutically acceptable salt, solvate, stereoisomer, or tautomer thereof, is selected from the group consisting of:

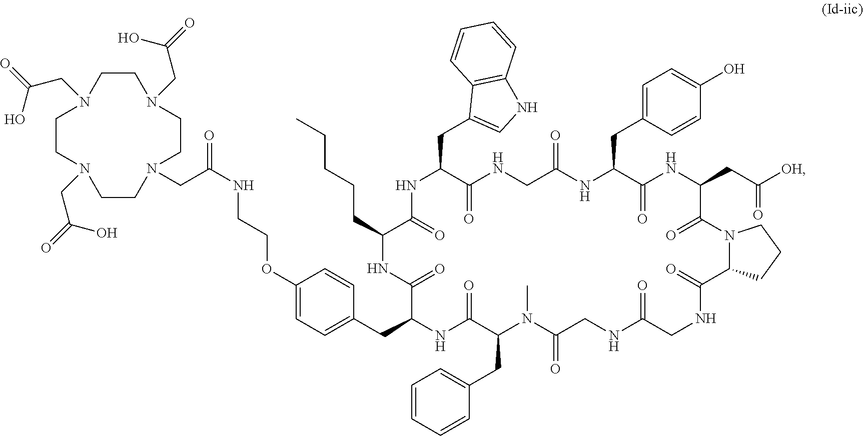 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O]

wherein the compound of formula (Id-iic) is radiolabeled with ²²⁵Ac,

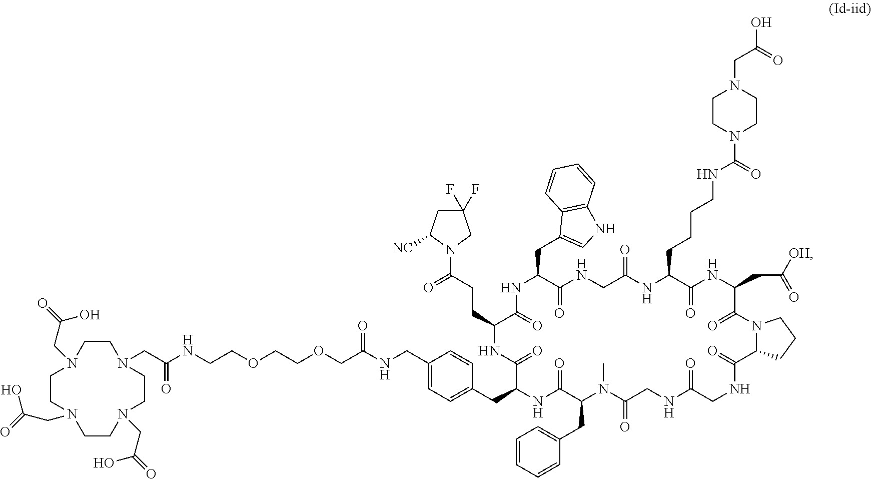 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@@H]1Cc1ccccc1]

wherein the compound of formula (Id-iid) is radiolabeled with ²²⁵Ac,

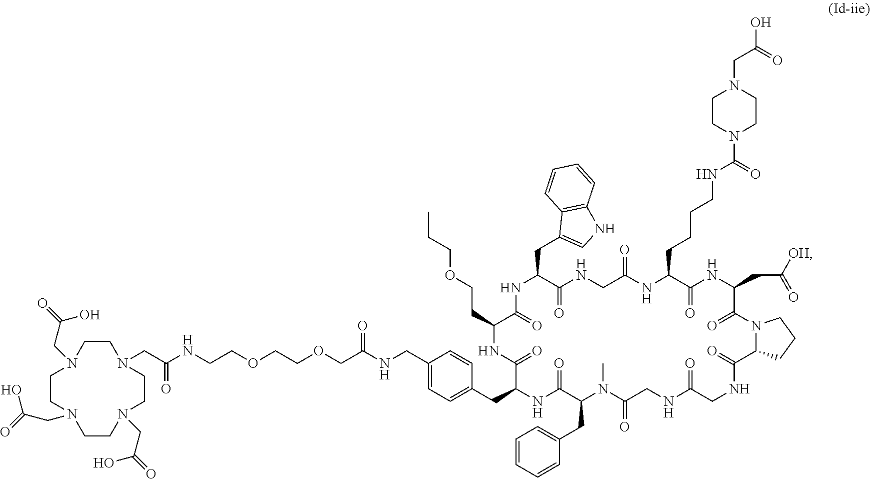 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iie) is radiolabeled with ²²⁵Ac, and

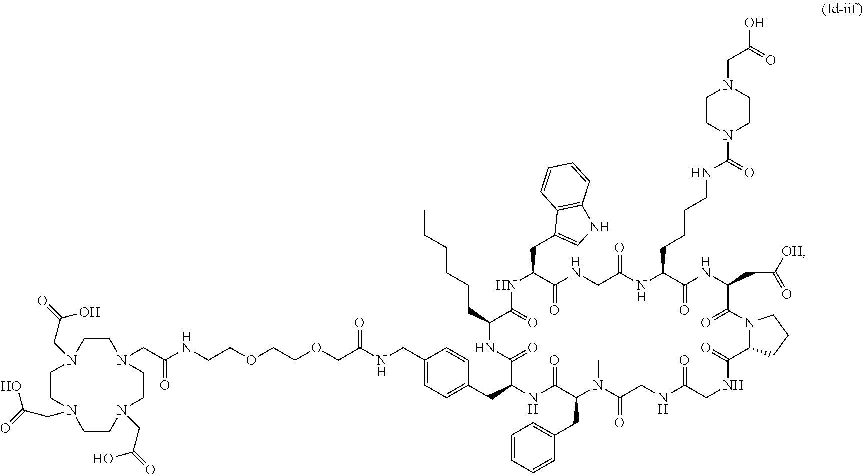 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iif) is radiolabeled with ²²⁵Ac.

In an embodiment of formula (Id), the compound of formula (Id), or a pharmaceutically acceptable salt, solvate, stereoisomer, or tautomer thereof, is selected from the group consisting of:

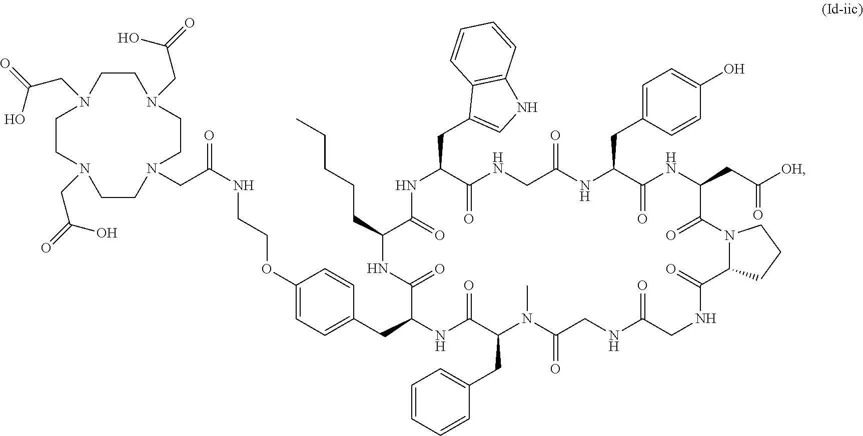 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O]

wherein the compound of formula (Id-iic) is radiolabeled with ⁶⁸Ga,

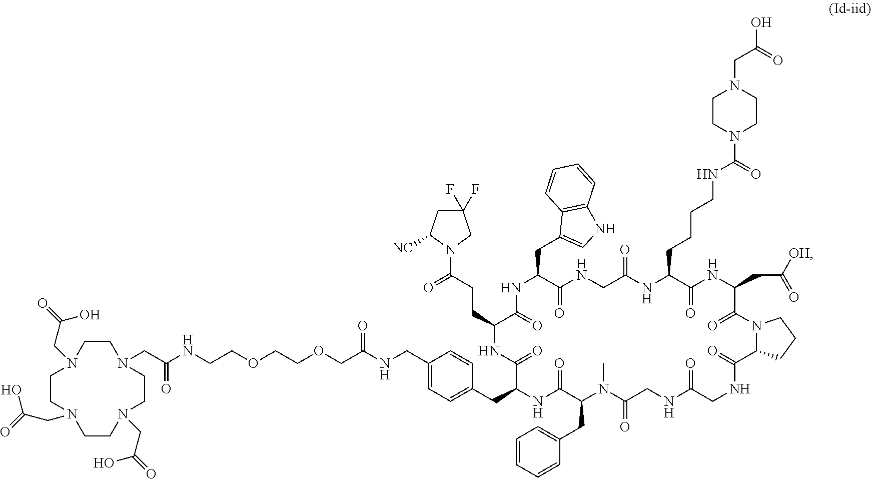 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@@H]1Cc1ccccc1]

wherein the compound of formula (Id-iid) is radiolabeled with ⁶⁸Ga,

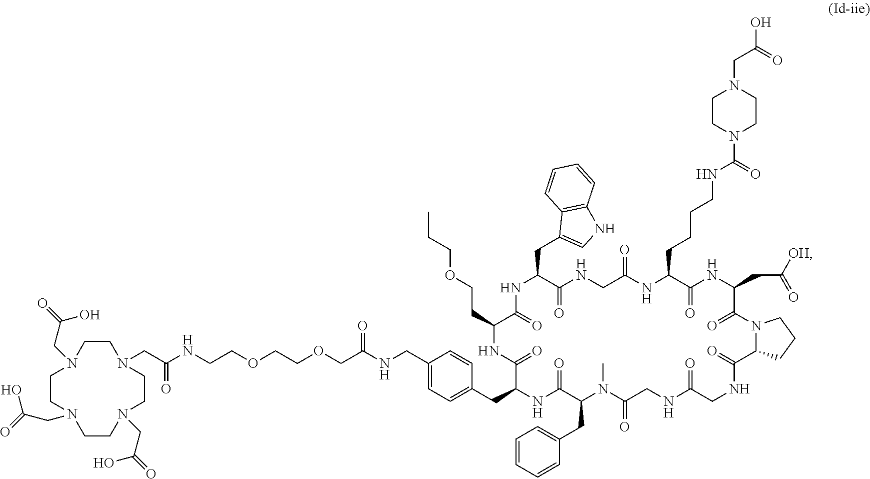 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iie) is radiolabeled with ⁶⁸Ga, and

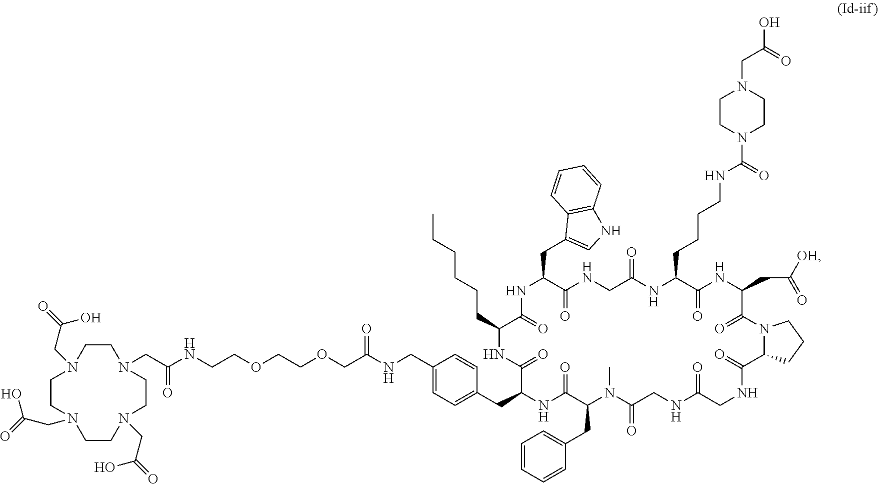 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

wherein the compound of formula (Id-iif) is radiolabeled with ⁶⁸Ga. In certain embodiments, the compound of formula (Id), or a pharmaceutically acceptable salt, solvate, stereoisomer, or tautomer thereof, is a compound selected from Examples B1-B40, C₁—C54, D1, D2, F1, F2, G1-G4, H1, and H2. In certain embodiments, the compound of formula (Id), or a pharmaceutically acceptable salt, solvate, stereoisomer, or tautomer thereof, is a compound selected from Examples B1-B40, C1-C54, D1, D2, F1, F2, G1-G4, H1, H2, I1, and J1. In certain embodiments, the compound of formula (Id), or a pharmaceutically acceptable salt, solvate, stereoisomer, or tautomer thereof, is a compound selected from Examples B1-B40, C1-C58, F1, F2, G1-G4, H1, and H2. In certain embodiments, the compound of formula (Id), or a pharmaceutically acceptable salt, solvate, stereoisomer, or tautomer thereof, is a compound selected from Examples B1-B40. In certain embodiments, the compound of formula (Id), or a pharmaceutically acceptable salt, solvate, stereoisomer, or tautomer thereof, is a compound selected from Examples C1-C58. In certain embodiments, the compound of formula (Id), or a pharmaceutically acceptable salt, solvate, stereoisomer, or tautomer thereof, is a compound selected from Examples C1-C54. In certain embodiments, the compound of formula (Id), or a pharmaceutically acceptable salt, solvate, stereoisomer, or tautomer thereof, is a compound selected from Examples G1-G4, H1, and H2.

The compound of formula (Id), or a pharmaceutically acceptable salt, solvate, stereoisomer, or tautomer thereof, may further comprise an albumin binding moiety attached to any of P, L¹ or M. The albumin binder may be as disclosed elsewhere herein. In some embodiments, an albumin binding moiety is attached to the compound of formula (Id), or a pharmaceutically acceptable salt, solvate, stereoisomer, or tautomer thereof, via a side chain of any of A¹-A¹⁰ of the cyclic peptide. In some embodiments, an albumin binding moiety is attached to the compound of formula (Id), or a pharmaceutically acceptable salt, solvate, stereoisomer, or tautomer thereof, via a functional group on L¹. In some embodiments, an albumin binding moiety is attached to the compound of formula (Id), or a pharmaceutically acceptable salt, solvate, stereoisomer, or tautomer thereof, via a functional group on M. For example, the compound of formula (Id), may be a compound of formula (Id-iii), (Id-iv), or (Id-v):

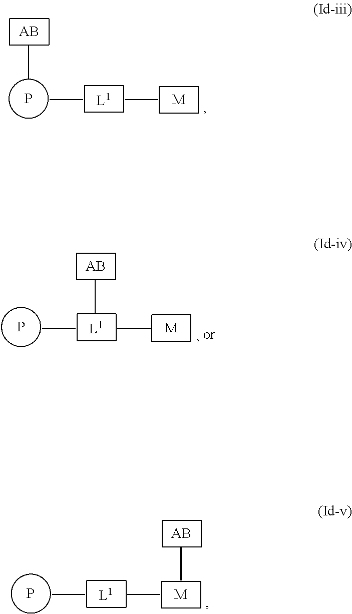

wherein AB is an albumin binder as disclosed elsewhere herein. In some embodiments of formulae (Id-iii), (Id-iv), and (Id-v), or a pharmaceutically acceptable salt, solvate, stereoisomer, or tautomer thereof, M is a chelator that is not radiolabeled with a radionuclide. In embodiments of formulae (Id-iii), (Id-iv), and (Id-v), or a pharmaceutically acceptable salt, solvate, stereoisomer, or tautomer thereof, M is a chelator that is radiolabeled with a radionuclide.

In various embodiments, the compound of formula (Id), or a pharmaceutically acceptable salt, solvate, stereoisomer, or tautomer thereof, is a compound of formula (Id-iii), or a pharmaceutically acceptable salt, solvate, stereoisomer, or tautomer thereof. In certain embodiments, the compound of formula (Id-iii), or a pharmaceutically acceptable salt, solvate, stereoisomer, or tautomer thereof, is a compound selected from Examples F1 and F2.

In certain embodiments, the FAP-targeting compound is a compound of formula (I):

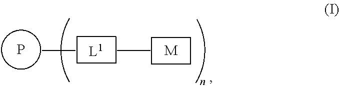

or a pharmaceutically acceptable salt, solvate, stereoisomer, or tautomer thereof,
where n is 2, and each L¹ is the same or different and each M is different. For example, in some embodiments, the compound of (I), or a pharmaceutically acceptable salt, solvate, stereoisomer, or tautomer thereof, is a compound of formula (Ie):

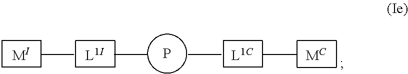

or a pharmaceutically acceptable salt, solvate, stereoisomer, or tautomer thereof
wherein:
P is a cyclic FAP-targeting peptide;
M^{I} is an imaging agent;
M^{C} is a chelator, optionally radiolabeled with a radionuclide;
L^{1I} is a bond or linker, connecting the imaging agent to P; and
L^{1C} is a bond or linker connecting the chelator to P;
wherein L^{1I} and L^{1C} are the same or different, and wherein the cyclic FAP-targeting peptide, imaging agent, chelator, radionuclide, and linker are as disclosed elsewhere herein.

In embodiments of the compound of formula (Ie), or a pharmaceutically acceptable salt, solvate, stereoisomer, or tautomer thereof, M^{I} is an imaging agent as previously described and M^{C} is a chelator radiolabeled with a radionuclide. In some embodiments of the compound of formula (Ie), or a pharmaceutically acceptable salt, solvate, stereoisomer, or tautomer thereof, MC is a chelator radiolabeled with a therapeutically active radionuclide. In some embodiments of the compound of formula (Ie), or a pharmaceutically acceptable salt, solvate, stereoisomer, or tautomer thereof, MC is a chelator radiolabeled with a diagnostically active radionuclide. In further embodiments of the compound of formula (Ie), or a pharmaceutically acceptable salt, solvate, stereoisomer, or tautomer thereof, the chelator is DOTA, and the radionuclide is ¹⁷⁷Lu or ⁶⁸Ga. In still further embodiments of the compound of formula (Ie), or a pharmaceutically acceptable salt, solvate, stereoisomer, or tautomer thereof, the imaging agent is Sulfo Cy5.

In certain embodiments, the compound of formula (Ie), or a pharmaceutically acceptable salt, solvate, stereoisomer, or tautomer thereof, is a compound selected from Examples D1 and D2.

The compound of formula (Ie), or a pharmaceutically acceptable salt, solvate, stereoisomer, or tautomer thereof, may further comprise an albumin binder as described above. In some embodiments, when the compound of formula (Ie), or a pharmaceutically acceptable salt, solvate, stereoisomer, or tautomer thereof, comprises an albumin binder, the albumin binder is attached to the compound via a side chain of any one of A¹-A¹⁰ of the cyclic peptide. In some embodiments, when the compound of formula (Ie), or a pharmaceutically acceptable salt, solvate, stereoisomer, or tautomer thereof, comprises an albumin binder, the albumin binder is attached to the compound via a functional group on L^{1I}. In some embodiments, when the compound of formula (Ie), or a pharmaceutically acceptable salt, solvate, stereoisomer, or tautomer thereof, comprises an albumin binder, the albumin binder is attached to the compound via a functional group on Lie. In some embodiments, when the compound of formula (Ie), or a pharmaceutically acceptable salt, solvate, stereoisomer, or tautomer thereof, comprises an albumin binder, the albumin binder is attached to the compound via a functional group on M^{I}. In some embodiments, when the compound of formula (Ie), or a pharmaceutically acceptable salt, solvate, stereoisomer, or tautomer thereof, comprises an albumin binder, the albumin binder is attached to the compound via a functional group on M^{C}.

In some embodiments, the compound of formula (I), is a compound of formula (If):

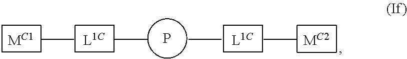

or a pharmaceutically acceptable salt, solvate, stereoisomer, or tautomer thereof,
wherein:
P is a cyclic FAP-targeting peptide;
M^{C1} is a chelator optionally radiolabeled with a radionuclide;
M^{C2} is a chelator optionally radiolabeled with a radionuclide; and
each L^{1C} is independently selected from a bond and a linker,
   and wherein the cyclic FAP-targeting peptide and each chelator, radionuclide, and linker is as previously described.

In some embodiments of the compound of Formula (If), or a pharmaceutically acceptable salt, solvate, stereoisomer, or tautomer thereof, M^{C1} and M^{C2} are the same. In some embodiments of the compound of Formula (If), or a pharmaceutically acceptable salt, solvate, stereoisomer, or tautomer thereof, M^{C1} and M^{C2} are different. In some embodiments of the compound of Formula (If), each Lie is the same. In some embodiments of the compound of Formula (If), or a pharmaceutically acceptable salt, solvate, stereoisomer, or tautomer thereof, each Lie is different.

The compound of formula (If), or a pharmaceutically acceptable salt, solvate, stereoisomer, or tautomer thereof, may further comprise an albumin binder as described elsewhere herein.

In some embodiments, the compound of formula (I), or a pharmaceutically acceptable salt, solvate, stereoisomer, or tautomer thereof, is a compound of formula (Ig):

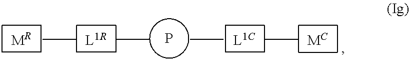

or a pharmaceutically acceptable salt, solvate, stereoisomer, or tautomer thereof,
wherein:
P is a cyclic FAP-targeting peptide;
M^{R} is a radionuclide;
M^{C} is a chelator optionally radiolabeled with a radionuclide;
L^{1R} is a bond or a linker; and
L^{1C} is a bond or a linker,
wherein the cyclic FAP-targeting peptide, radionuclide, chelator, and linker are as previously described.

In certain embodiments of formula (Ig), or a pharmaceutically acceptable salt, solvate, stereoisomer, or tautomer thereof, M^{R} is a radiohalogen and L^{1R} is a bond. In some embodiments of formula (Ig), or a pharmaceutically acceptable salt, solvate, stereoisomer, or tautomer thereof, when M^{R} is a radiohalogen and L^{1R} is a bond, then M^{C} is a chelator radiolabeled with a radionuclide other than a radiohalogen, and L^{1C} is a linker.

The compound of formula (Ig) may further comprise an albumin binder as described elsewhere herein.

In certain embodiments, the FAP-targeting compound is a compound of formula (Ib) or (Ic):

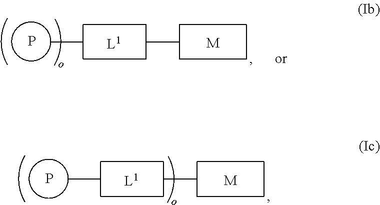

or a pharmaceutically acceptable salt, solvate, stereoisomer, or tautomer thereof,
wherein o is 2, each L¹ is the same or different and each P is different. For example, in some embodiments, the compound of formula (Ib) is a compound of formula (Ih):

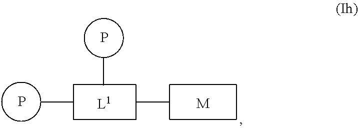

or a pharmaceutically acceptable salt, solvate, stereoisomer, or tautomer thereof,
wherein:
each P is independently a cyclic FAP-targeting peptide;
L¹ is a bond or a linker; and
M is an imaging agent, a chelator optionally radiolabeled with a radionuclide, or a radionuclide, and wherein the FAP-targeting cyclic peptides, linker, imaging agent, chelator, and radionuclide are as described elsewhere herein.

In some embodiments of the compound of formula (Ih), or a pharmaceutically acceptable salt, solvate, stereoisomer, or tautomer thereof, each P is the same. In some embodiments of the compound of formula (Ih), or a pharmaceutically acceptable salt, solvate, stereoisomer, or tautomer thereof, each P is different. Each FAP-targeting cyclic peptide of the compound of formula (Ih) may be different by one or more amino acids, two or more amino acids, three or more amino acids, four or more amino acids, five or more amino acids, six or more amino acids, seven or more amino acids, eight or more amino acids, or nine or more amino acids. In some embodiments, the FAP-targeting cyclic peptides of the compound of formula (Ih) are different by no more than one amino acid, no more than two amino acids, no more than three amino acids, or no more than four amino acids.

In various embodiments of the compound of formula (Ih), or a pharmaceutically acceptable salt, solvate, stereoisomer, or tautomer thereof, the compound further comprises an albumin binder (as described elsewhere herein).

In some embodiments, the compound of formula (Ic) is a compound of formula (Ii):

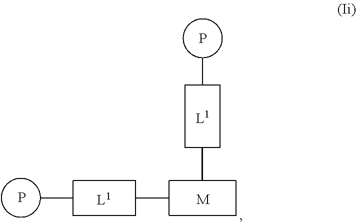

or a pharmaceutically acceptable salt, solvate, stereoisomer, or tautomer thereof,
wherein:
each P is independently a cyclic FAP-targeting peptide;
each L¹ is selected from a bond and a linker; and
M is an imaging agent, a chelator optionally radiolabeled with a radionuclide, or a radionuclide, and wherein the FAP-targeting cyclic peptides, linker, imaging agent, chelator, and radionuclide are as described elsewhere herein.

In some embodiments of the compound of formula (Ii), or a pharmaceutically acceptable salt, solvate, stereoisomer, or tautomer thereof, each P is the same. In some embodiments of the compound of formula (Ii), or a pharmaceutically acceptable salt, solvate, stereoisomer, or tautomer thereof, each P is different. In some embodiments of the compound of Formula (Ih), or a pharmaceutically acceptable salt, solvate, stereoisomer, or tautomer thereof, each L¹ is the same. In some embodiments of the compound of Formula (Ii), or a pharmaceutically acceptable salt, solvate, stereoisomer, or tautomer thereof, each L¹ is different.

Each FAP-targeting cyclic peptide of the compound of formula (Ii) may be different by one or more amino acids, two or more amino acids, three or more amino acids, four or more amino acids, five or more amino acids, six or more amino acids, seven or more amino acids, eight or more amino acids, or nine or more amino acids. In some embodiments, the FAP-targeting cyclic peptides of the compound of formula (Ii) are different by no more than one amino acid, no more than two amino acids, no more than three amino acids, or no more than four amino acids.

In various embodiments of the compound of formula (Ii), the compound further comprises an albumin binder. The albumin binder may be as described elsewhere herein.

In any of formulae (Id)-(Ii), and any other combination of P, L¹, and M encompassed by the present disclosure, the cyclic peptide may be further connected to various moieties that may increase or supplement the activity of the disclosed FAP-targeting compounds. Moieties that may be connected to any of the compounds described above include, but are not limited to, albumin binders, additional FAP-targeting agents, additional imaging agents, and cytotoxic drugs.

### Methods of Synthesis

The compounds described herein may be synthesized by many techniques that are known to those skilled in the art. In some aspects, the present disclosure provides a method of chemically synthesizing a radioligand peptide of the present disclosure. In some embodiments, a portion of the peptide is recombinantly synthesized, rather than chemically synthesized. In some embodiments, methods of producing a radioligand peptide include cyclizing the peptide portion after all the constituents have been attached. In other embodiments, methods of producing a radioligand peptide include cyclizing the peptide prior to attachment of all the constituents to one another. In particular embodiments, cyclization is accomplished via any of the various methods described herein.

In some embodiments, the peptide is first synthesized and then covalently attached to the linker. The chelator may be attached to the linker before or after attachment of the peptide. For example, in some embodiments, the peptide is attached to the linker to form a linker-peptide intermediate that is then attached to the chelator. In other embodiments, the chelator is attached to the linker to form a linker-chelator intermediate that is then attached to the peptide.

In some embodiments, one or more of the amino acid residues or amino acid are covalently attached to one another and then attached to the linker at an intermediate oligomer stage before attaching additional amino acids and cyclization to form a peptide of the disclosure. In such embodiments, the intermediate oligomer may comprise the linker and a fragment of the peptide sequence or the linker, the chelator, and a fragment of the peptide sequence. As will be appreciated by one of ordinary skill in the art, the fragment of the peptide sequence may have a sequence length any number of amino acids shorter than the length of the complete peptide sequence. For example, if the peptide sequence is 10 residues long, then a fragment of that sequence may be 1, 2, 3, 4, 5, 6, 7, 8, or 9 residues long. Illustrative synthetic methods are described in the Examples.

### Pharmaceutical Compositions

The present disclosure further provides a pharmaceutical composition comprising FAP-targeting radioligand described herein. In particular, a pharmaceutical composition of the present disclosure includes one or more radioligand peptides disclosed herein and a pharmaceutically acceptable carrier, diluent, or excipient. The pharmaceutically acceptable carrier, diluent or excipient may be a solid, semi-solid or liquid filler, diluent, encapsulating material or formulation auxiliary of any type.

The pharmaceutical compositions may be administered parenterally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered by parenteral administration. In particular, a pharmaceutical composition of the present disclosure may be formulated for and administered by intravenous administration.

Radioligands of the present disclosure may be prepared and/or formulated as pharmaceutically acceptable salts and/or other forms thereof or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts can be found in Remington: The Science and Practice of Pharmacy, 21st Edition, Lippincott Wiliams and Wilkins, Philadelphia, Pa., 2006.

In certain aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain additives such as preservatives, wetting agents, emulsifying agents, chelating agents, buffering agents, and dispersing agents.

In some embodiments, the pharmaceutical composition comprises a chelating agent to sequester internally deposited radionuclides. Any of the chelating agents listed in Tables 3-7 above may be included in pharmaceutical compositions comprising the FAP-targeting radioligand. In various embodiments, the pharmaceutical composition comprises DTPA. Additional exemplary chelating agents that may be included in pharmaceutical compositions of the present disclosure are described in Holik, et al. “The Chemical Scaffold of Theranostic Radiopharmaceuticals: Radionuclide, Bifunctional Chelator, and Pharmacokinetics Modifying Linker.” Molecules 27.10 (2022): 3062 and Kostelnik, Thomas I., and Chris Orvig. “Radioactive main group and rare earth metals for imaging and therapy.” Chemical reviews 119.2 (2018): 902-956, both of which are incorporated by reference in their entireties.

In some embodiments, the pharmaceutical composition comprises one or more buffering agents to maintain a pH of about 3 to 5. Suitable buffering agents include, but are not limited to acetate, citrate, Tris, lactate, and tartrate, and the acid forms thereof.

Pharmaceutical compositions including the FAP-targeting radioligand of the instant disclosure may further comprise a stabilizer, such as, for example, a free radical scavenger, in order to prevent autoradiolysis of the inventive radioligand. Suitable stabilizers for inclusion in the disclosed pharmaceutical compositions include, but are not limited to, 2,5-dihydroxybenzoic acid or salts thereof, ascorbic acid or salts thereof, gentisic acid or salts thereof, methionine, histidine, melatonine, N-acetylmethionine, ethanol, an amino acid infusion solution, or any combination thereof. In some embodiments, the pharmaceutical composition includes a gentisic acid stabilizer. In some embodiments, the pharmaceutical composition includes an ascorbic acid stabilizer. In some embodiments, the pharmaceutical composition include stabilizer including gentisic acid and ascorbic acid.

In some embodiments, injectable compositions including a FAP-targeting radioligand are administered by infusion. For example, a composition of the present disclosure may be administered to a subject by an infusion ranging from about 1 to about 120 minutes in duration. In some embodiments, a composition of the present disclosure may be administered to a subject by an infusion ranging from about 1 to about 60 minutes in duration. In some embodiments, a composition of the present disclosure may be administered to a subject by an infusion ranging from about 1 to about 30 minutes in duration. In some embodiments, a composition of the present disclosure may be administered to a subject by an infusion ranging from about 1 to about 20 minutes in duration. In some embodiments, a composition of the present disclosure may be administered to a subject by an infusion ranging from about 1 to about 10 minutes in duration. In some embodiments, a composition of the present disclosure may be administered to a subject by an infusion ranging from about 5 to about 10 minutes in duration.

In some embodiments, the pH of the disclosed pharmaceutical compositions ranges from about 3 to about 11. The pH of the compositions may, for example, range from about 3 to about 7 or from about 3 to about 5.

The total daily usage of the FAP-targeting ligands (e.g., FAP-targeting radioligands) and compositions of the present disclosure can be decided by the attending physician within the scope of reasonable medical judgment. The specific therapeutically effective dose level for any particular subject will depend upon a variety of factors including: a) the disorder being treated and the severity of the disorder; b) activity of the specific compound employed; c) the specific composition employed; d) the age, body weight, general health, sex and diet of the patient; e) the time route, and mechanism of administration; f) the rate of excretion of the specific compound employed; g) the duration of the treatment (including the number of cycles in a treatment); h) administration of combination partners (e.g., chemotherapies and/or imaging agents), and like factors well known in the medical arts. Provided herein are radiopharmaceuticals or radioligands targeting FAP. In particular embodiments, the dosing of a FAP-targeting radioligands of the present disclosure to be administered to a human or other mammal host in single or divided doses may be referred to as administered activity to be delivered to the subject (e.g., human or other mammal host). Administered activity of radiopharmaceuticals is given in units of radioactive disintegrations per unit time (SI units Becquerels (BQ), imperial units Curies (Ci)). The total dose will vary depending on the purpose, i.e., for imaging and/or therapy, and the number of cycles of administration. Dosing may also be based on energy deposited per unit of mass, i.e., the absorbed dose, usually given in J/kg or Gy).

The dosing of the radio-labeled compounds will also be determined by the particular radionuclide, and whether said radionuclide is a P-emitter (can also be referred to in the art as a beta-minus emitter) (e.g., ⁹Y, ¹³¹I, ¹⁵³Sm, or ¹⁷⁷Lu) or an a-emitter (e.g., ²¹¹As, ²¹²Pb, ²¹²Bi, ²²³Ra, ²²⁵Ac, or ²²⁷Th).

In some embodiments, the total dose (over the course of a treatment regimen) of the FAP-targeting ligand radiolabeled with a β-emitter such as, e.g., ¹⁷⁷Lu, is from about 1 GBq to about 200 GBq. In some embodiments, the FAP-targeting radioligand comprising a β-emitter is administered in a total dose to deliver from 40 to 100 GBq of radiation. In some embodiments, the FAP-targeting radioligand comprising the p-emitter is administered in a single dose (once within a 24-hour period) to deliver from about 1 to about 20 GBq of radiation. In some embodiments, the FAP-targeting radioligand comprising the p-emitter is administered in a single dose (once within a 24-hour period) to deliver from about 3 to about 15 GBq of radiation. In some embodiments, the FAP-targeting radioligand comprising the p-emitter is administered in a single dose (once within a 24-hour period) to deliver from about 5 to about 10 GBq of radiation.

In some embodiments, the total dose (over the course of a treatment regimen) of the FAP-targeting ligand radiolabeled with an a-emitter, e.g., ²²′Ac, is from about 1 MBq to about 100 MBq. In some embodiments, the FAP-targeting radioligand comprising an a-emitter is administered in a total dose of from about 20 to about 80 MBq of radiation. In some embodiments, the FAP-targeting radioligand comprising the a-emitter is administered in a single dose (once within a 24-hour period) to deliver from about 1 to about 40 MBq of radiation. In some embodiments, the FAP-targeting radioligand comprising the a-emitter is administered in a single dose (once within a 24-hour period) to deliver from about 5 to about 40 MBq of radiation. In some embodiments, the FAP-targeting radioligand comprising the a-emitter is administered in a single dose (once within a 24-hour period) to deliver from about 5 to about 25 MBq of radiation.

### Combination Therapies

Also provided herein are combinations (e.g., combination therapies) comprising at least one FAP-targeting ligand of the present disclosure and one or more other therapeutically active agents. The disclosed pharmaceutical combinations may be used in the treatment or prevention of FAP-related diseases, such as, for example, cancer. The FAP-targeting ligand of the present disclosure, when radiolabeled with a suitable imaging and/or therapeutic radionuclide, may be used in the treatment of a cancer wherein the subject is also receiving standard of care treatment or other chemotherapies approved for the treatment of the cancer. Suitable pharmaceutical combinations may include a FAP-targeting compound described herein and one or more immune checkpoint inhibitors, such as, for example, PD-1 and PD-L1 inhibitors, CTLA-4 inhibitors, and LAG-3 inhibitors; selected estrogen receptor modulators; cyclin-dependent kinase (CDK) inhibitors, such as, e.g., CDK4 and CDK6 inhibitors; steroidal and nonsteroidal aromatase inhibitors; hormone receptor antagonists and inhibitors; antineoplastic agents; antimitotic agents; mTOR kinase inhibitors; antimetabolites; antifolates; DNA intercalators; tyrosine kinase inhibitors; and/or nonsteroidal anti-inflammatory drugs (NSAIDs).

### Methods of Treatment

The present disclosure also provides a method of treating one or more FAP-related diseases or disorders in a subject in need thereof, the method comprising administering a therapeutically effective amount of a radioligand described herein to the subject. The radioligand of the present disclosure may be administered to a subject having any FAP-related disease or disorder, including, but not limited to: proliferation diseases, such as, for example, cancer; tissue remodeling and/or chronic inflammation, such as, for example, fibrotic disease, wound healing, keloid formation, osteoarthritis, rheumatoid arthritis, and related disorders involving cartilage degradation; and endocrinological disorders, such as, for example, glucose metabolism disorders. In certain embodiments, the FAP-related disease or disorder is cancer.

In an aspect, provided herein is a method treating cancer in a subject in need thereof comprising administering a therapeutically effective amount of, e.g., a compound of any of Formulae (I), (Ia), (Ib), (Ic), (Id) and (I-i), particularly a radiolabeled compound of any of Formulae (I), (Ia), (Ib), (Ic), (Id) and (I-i). In an embodiment, the cancer overexpresses FAP.

In some aspects, the FAP-targeting radioligand or a pharmaceutical composition thereof is used as a curative or adjuvant cancer treatment. In other aspects, the radioligand peptide or composition thereof is used as a palliative treatment. Methods of palliative treatment using the disclosed radioligands include local disease control and/or symptomatic relief.

The FAP-targeting radioligands described herein may be used as the primary therapy for treatment of FAP-related disease or disorders, such as cancer. Alternatively, the FAP-targeting radioligands of the instant disclosure may be the secondary, tertiary, or final therapy for a FAP-related disease or disorder.

The FAP-targeting radioligands of the instant disclosure may be used in methods of treating cancers such as, for example, bladder cancer (including, e.g., urothelial carcinoma), brain cancer (including, e.g., glioblastoma), breast cancer (including, e.g., triple-negative breast cancer (TNBC), hormone receptor-positive/HER2-negative breast cancer (such as HR+/HER2− ductal breast cancer, HR+/HER2− lobular breast cancer, HR+/HER2− ductal and lobular breast cancer (BC), HR+/HER2− ductal or lobular breast cancer)), cholangiocarcinoma, colon cancer, colorectal cancer, endocrine cancer, epithelial cancer, esophageal cancer, gastric cancer, head/neck cancer, mesothelioma, nasopharyngeal cancer, lung cancer, ovarian cancer, pancreatic cancer, prostate cancer, salivary gland cancer, testicular cancer, thyroid cancer, and sarcoma. In some embodiments, the FAP-targeting radioligand is used in a method of treating a bladder cancer, breast cancer, cholangiocarcinoma, colon cancer, colorectal cancer, endocrine cancer, epithelial cancer, glioblastoma, head/neck cancer, mesothelioma, nasopharyngeal cancer, lung cancer, ovarian cancer, pancreatic cancer, prostate cancer, testicular cancer, thyroid cancer, and/or sarcoma. In particular embodiments, the FAP-targeting radioligand is used in a method of treating breast cancer, colorectal cancer, non-small cell lung cancer (NSCLC), and/or pancreatic ductal adenocarcinoma (PDAC) (e.g., breast cancer, NSCLC, or PDAC).

In some embodiments, the FAP-targeting radioligand therapeutic of the present disclosure are used in methods of treating cancer, chronic inflammation, atherosclerosis, fibrosis, tissue remodeling and keloid disorder.

In an embodiment, FAP-targeting radioligand therapeutic of the present disclosure are used in methods of treating cancer. In certain embodiments, the cancer is a solid tumor. In certain embodiments, the cancer is characterized by FAP expression. In certain embodiments, the cancer is characterized by FAP over-expression.

In further embodiments, the cancer is selected from the group consisting of adenocarcinoma, breast cancer (including, e.g., triple-negative breast cancer (TNBC)), brain cancer (including, e.g., glioblastoma), pancreatic cancer (including, e.g., pancreatic ductal adenocarcinoma (PDAC)), small intestine cancer, cholangiocarcinoma, colon cancer, rectal cancer, colorectal cancer (e.g., colorectal cancer with microsatellite instability-high (MSI-H) or mismatch repair deficiency/deficient (dMMR)), gastric cancer, lung cancer, head and neck cancer, melanoma, ovarian cancer, hepatocellular carcinoma, esophageal cancer, hypopharynx cancer, nasopharynx cancer, larynx cancer, myeloma cells, fibrosarcoma, bladder cancer (including e.g., urothelial carcinoma), giant cell carcinoma, squamous cell carcinoma (e.g., squamous cell carcinoma of head and neck (SCCHN)), renal cell carcinoma, neuroendocrine tumor, oncogenic osteomalacia, bone and connective tissue sarcomas, soft-tissue sarcomas, CUP (carcinoma of unknown primary), thymus carcinoma, desmoid tumors, glioma, astrocytoma, cervix carcinoma, prostate cancer, and salivary gland cancer.

In certain embodiments, the FAP-targeting radioligand therapeutic described herein are used in methods of treating breast cancer, colorectal cancer, epithelial cancer, ovarian cancer, prostate cancer, pancreatic cancer, kidney cancer, lung cancer, melanoma, fibrosarcoma, bone and connective tissue sarcomas, soft-tissue sarcomas, renal cell carcinoma, giant cell carcinoma, squamous cell carcinoma, and/or adenocarcinoma.

In some embodiments, methods of treating FAP-related diseases and disorders include co-administration of a disclosed radioligand with an additional therapy. In particular, when the FAP-related disease or disorder is cancer, methods may include combining a FAP-targeting radioligand therapeutic with one or more anti-cancer therapies (i.e., an anti-cancer therapeutic agent or a chemotherapeutic). Any known anticancer therapy may be combined with the radioligand of the present disclosure in order to provide curative, adjuvant, or palliative treatment to a subject in need thereof. For example, suitable anticancer therapies that may be combined with the presently disclosed radioligand therapeutic include, but are not limited to, surgery, anticancer drugs, PARP inhibitors, inhibitors of signaling pathways and mechanisms leading to repair of DNA single and double strand breaks, such as nuclear factor-kappa B signaling, immunomodulators, immune checkpoint inhibitors, antibodies capable of inducing antibody-dependent cellular cytotoxicity, T-cell or NK cell engagers, and cellular therapies using expanded autologous or allogeneic cells. One of ordinary skill in the art would be capable of discerning a suitable anticancer therapy to be used in combination with the radioligand therapeutic depending on a number of factors including, cancer type, stage, and location, as well as the general health and physical characteristics of the patient, e.g., weight, age, sex, etc.

The additional anticancer therapy may be administered concurrent with, prior to, or after administration of the FAP-targeting radioligand therapeutic. In some embodiments, the administration schedule involves administering the different agents in an alternating fashion, such as alternating days, weeks, or months. In other embodiments, the compounds may be delivered before and during, or during and after, or before and after treatment with one or more other anticancer therapies. In some embodiments, more than one additional anticancer therapy is administered to a subject. For example, the subject may receive the presently described FAP-targeting radioligand therapeutic, in combination with surgery and at least one other anticancer drug. Alternatively, the compound may be administered in combination with more than one additional anticancer drug or therapy.

In any of the foregoing methods, administration of the FAP-targeting radioligand therapeutic to the subject may be conducted parenterally, in particular, intravenously, but other routes of administration are not excluded. Other routes of administration include, but are not limited to, subcutaneous, intramuscular, intraperitoneal, transdermal, topical, buccal, or oral routes. In certain embodiments, the radioligand therapeutic may be administered as close to the disease site as possible, such as in the diseases tissue, surrounding tissue, or nearby blood vessels.

The FAP-targeting radioligand therapeutic may be dosed according to the total radiation to be administered to the subject in need thereof, and as described herein.

In some embodiments, the FAP-targeting radioligand therapeutic is administered on alternate days, weeks, or months. For example, FAP-targeting radioligand therapeutic described herein may be administered every two days, or every three days, or every four days, or every five days, or every six days, or every week, or every month. The FAP-targeting radioligands described herein may be administered every two weeks, or every three weeks, or every four weeks, or every five weeks, or every six weeks, or every seven weeks, or every eight weeks, or every nine weeks, or every ten weeks.

In some embodiments, the FAP-targeting radioligand is administered once about every 2 weeks to 10 weeks. In some embodiments, the FAP-targeting radioligand is administered once about every 2 weeks to 6 weeks. In some embodiments, the FAP-targeting radioligand is administered once about every 2 weeks to 4 weeks. In some embodiments, the FAP-targeting radioligand is administered once about every 3 weeks to 10 weeks. In some embodiments, the FAP-targeting radioligand is administered once about every 3 weeks to 6 weeks. In some embodiments, the FAP-targeting radioligand is administered once about every 3 weeks to 4 weeks. In some embodiments, the FAP-targeting radioligand is administered once about every 4 weeks to 6 weeks (e.g., once about every four weeks, once about every 5 weeks, or once about every 6 weeks). In some embodiments, the FAP-targeting radioligand is administered once about every 5 weeks to 6 weeks. In some embodiments, the FAP-targeting radioligand is administered once about every 6 weeks to 8 weeks. In some embodiments, the FAP-targeting radioligand is administered once about every 6 weeks to 7 weeks. In some embodiments, the present disclosure provides a method of treating a subject afflicted with a disease or disorder using the FAP-targeting therapeutics described herein, wherein the treatment is radionuclide therapy. Radionuclide therapy is based on different forms of radiation emitted by a radionuclide, including, but not limited to, radiation of photons, radiation of electrons, such as, for example, β-particles and auger-electrons, radiation of protons, radiation of neutrons, radiation of positrons, radiation of α-particles or an ion beam. Accordingly, depending on the kind of particle or radiation emitted by the radionuclide, radionuclide therapy can be distinguished as photon radionuclide therapy, electron radionuclide therapy (e.g., β-particle radionuclide therapy), proton radionuclide therapy, neutron radionuclide therapy, positron radionuclide therapy, α-particle radionuclide therapy, or ion beam radionuclide therapy. Methods of the present disclosure that utilize radionuclide therapy for treatment of a subject may rely on any one or more of these forms of radiotherapy. In particular, the FAP-targeting radioligand therapeutic of the present disclosure may be used to provide any one or more of the above forms of radionuclide therapy to a subject in need thereof. In certain embodiments, α-emitting radionuclide therapy may be useful. In certain other embodiments, β-emitting radionuclide therapy may be useful.

In some embodiments, methods may include the use of certain techniques or systems commonly used in a clinical setting to accelerate and/or increase the effectiveness of the radiation therapy. For example, as oxygen is known in the art to be a potent radiosensitizer by readily forming free radicals, the presently described radioligand may be used in conjunction with high-pressure oxygen tanks, blood substitutes having increased oxygen content, and hypoxic cell radiosensitizers in some cases.

The compounds disclosed herein, including the compounds of Formula (I), (Ia), (Ib), (Ic), or (Id), or a pharmaceutically acceptable salt, solvate, stereoisomer, or tautomer thereof, exhibit strong binding to human and/or mouse FAP, i.e., exhibit a dissociation constant (K_{D}) of about 10 nM, about 5 nM, or about 1 nM or less as measured by surface plasmon resonance (SPR) at a temperature of 25° C. (see, e.g., the method described particularly in Example I, part I, and values in Table 7.3).

In some embodiments, any one of the compounds as disclosed herein exhibit a K_{D} of about 1 nM or less, about 0.9 nM or less, about 0.8 nM or less, about 0.7 nM or less, about 0.6 or less, about 0.5 nM or less, about 0.4 nM or less, about 0.3 nM or less, about 0.2 nM or less, about 0.1 nM or less, about 0.05 nM or less, about 0.04 nM or less, about 0.03 nM or less, about 0.02 nM or less, about 0.01 nM or less, about 0.005 nM or less, or about 0.001 nM or less as measured by surface plasmon resonance (SPR) at a temperature of 25° C. In some embodiments, the K_{D} is about 0.9 nM or less. In some embodiments, the K_{D} is about 0.8 nM or less. In some embodiments, the K_{D} is about 0.7 nM or less. In some embodiments, the K_{D} is about 0.7 nM or less. In some embodiments, the K_{D} is about 0.6 nM or less. In some embodiments, the K_{D} is about 0.5 nM or less. In some embodiments, the K_{D} is about 0.4 nM or less. In some embodiments, the K_{D} is about 0.3 nM or less. In some embodiments, the K_{D} is about 0.2 nM or less. In some embodiments, the K_{D} is about 0.1 nM or less. In some embodiments, the K_{D} is about 0.05 nM or less. In some embodiments, the K_{D} is about 0.04 nM or less. In some embodiments, the K_{D} is about 0.03 nM or less. In some embodiments, the K_{D} is about 0.02 nM or less. In some embodiments, the K_{D} is about 0.01 nM or less. In some embodiments, the K_{D} is about 0.005 nM or less. In some embodiments, the K_{D} is about 0.001 nM or less.

In some embodiments, any one of the compounds as disclosed herein exhibit a K_{D} between about 10 nM and about 0.001 nM as measured by SPR at a temperature of 25° C. In some embodiments, any one of the compounds as disclosed herein exhibit a K_{D} between about 5 nM and about 0.001 nM as measured by SPR at a temperature of 25° C. In some embodiments, any one of the compounds as disclosed herein exhibit a K_{D} between about 1 nM and about 0.001 nM as measured by SPR at a temperature of 25° C.

The compounds disclosed herein, including the compounds of Formula (I), (Ia), (Ib), (Ic), or (Id), or a pharmaceutically acceptable salt, solvate, stereoisomer, or tautomer thereof, also exhibit high potency against FAP (inhibit human and/or mouse FAP enzymatic activity), i.e., exhibit an IC₅₀ for human FAP of about 10 nM or less, about 5 nM or less, or about 1 nM or less as measured by an Enzymatic FAP competition assay (see, e.g., the method described particularly in Example I, part II, and values in Table 7.3).

In some embodiments, any one of the compounds as disclosed herein exhibit an IC₅₀ for human FAP between about 10 nM and about 0.001 nM as measured an Enzymatic FAP competition assay. In some embodiments, any one of the compounds as disclosed herein exhibit an IC₅₀ for human FAP between about 5 nM and about 0.001 nM as measured an Enzymatic FAP competition assay. In some embodiments, any one of the compounds as disclosed herein exhibit an IC₅₀ for human FAP between about 1 nM and about 0.001 nM as measured an Enzymatic FAP competition assay.

### Methods of Imaging

Also provided herein is a method of imaging a disease or disorder associated with fibroblast activation protein using the disclosed FAP-targeting radioligands. In some aspects, the method includes administering a detectably effective amount (an amount effective for imaging) of a radioligand of the present disclosure, or a pharmaceutical composition thereof, to a subject.

In some embodiments, the method includes imaging one or more cells, tissues, or organs, including, but not limited to, kidney tissue, prostate tissue, brain tissue, vascular tissue, and tumor tissue.

The FAP-targeting compounds described herein are suitable for imaging any physiological process or feature in which FAP is involved, such as for identifying areas of tissues or targets which exhibit or express high concentrations (e.g., over-expression) of FAP. Exemplary physiological processes in which FAP is involved include, but are not limited to, proliferation diseases, tissue remodeling and/or chronic inflammation, and endocrinological disorders.

In some embodiments, the method includes imaging one or more cells, tissues, or organs implicated in a proliferation disease. In some embodiments, the proliferation disease is cancer. For example, one or more cells, tissues, or organs suitable for imaging with the radioligand peptides described herein may be implicated in cancers such as, for example, breast cancer, colorectal cancer, epithelial cancer, ovarian cancer, prostate cancer, pancreatic cancer, kidney cancer, lung cancer, melanoma, fibrosarcoma, bone and connective tissue sarcomas, renal cell carcinoma, giant cell carcinoma, squamous cell carcinoma, and adenocarcinoma.

In some embodiments, the method includes imaging one or more cells, tissues, or organs implicated in tissue remodeling and/or chronic inflammation, including, but not limited to, fibrotic disease, wound healing, keloid formation, osteoarthritis, rheumatoid arthritis, and relating disorders involving cartilage degradation.

In some aspects, methods of imaging one or more cells, tissues, or organs with FAP-targeting radioligands of the present disclosure further include detecting and/or assessing a disease or disorder in the subject. Detecting and/or assessing a disease or disorder may be useful in diagnosis, prognosis, and prediction of a disease or disorder in the subject. For example, FAP-targeting radioligands disclosed herein may be used to diagnose a disease or disorder afflicting the subject, to determine the subject's risk of developing a disease or disorder, to assess the evolution of a disease or disorder in the subject, or to predict a subject's response to a certain therapy given to treat the disease or disorder.

In some embodiments, the FAP-targeting radioligands are detectable by positron emission tomography (PET) or single photon emission computed tomography (SPECT). In some embodiments, the FAP-targeting radioligands are detectable by scintigraphy.

The FAP-targeting radioligand or a pharmaceutical composition thereof may be administered to the subject for imaging purposes via parenteral administration, but other routes of administration are not excluded. Other routes of administration include, but are not limited to, subcutaneous, intravenous, intramuscular, intraperitoneal, transdermal, or oral routes. The amount of a radioligand described herein, or a pharmaceutically acceptable salt or composition thereof to be administered to a subject may be determined by a person of skill in the art taking into account the disease or condition being treated, including its locale, and factors including age, weight, sex, and the like.

### Theragnostic Methods

The present disclosure further provides a method to both image and treat or prevent a disease or disorder associated with fibroblast activation protein using the disclosed FAP-targeting ligands and radioligands. As used herein, a method of imaging and treating a disease or disorder may be referred to as a theragnostic method.

In some embodiments, the theragnostic method includes administering a diagnostically effective and therapeutically effective dose of a FAP-targeting compound described herein to a subject in need thereof. In some embodiments, a FAP-targeting compound may be used to image one or more tissues, cells, or organs implicated in a FAP-related disease and to treat the FAP-related disease in those tissues, cells, or organs. In particular, a theragnostic method may be used for both imaging and treatment of cancer in a subject in need thereof.

In some embodiments, one FAP-targeting compound is used in the theragnostic method described herein (i.e., the same FAP-targeting radioligand is used in both imaging and therapeutic methods). Accordingly, in some embodiments, the FAP-targeting compound is both diagnostically and therapeutically active.

The FAP-targeting compound used in a theragnostic method may be complexed to one radionuclide that is diagnostically and therapeutically active, or more than one radionuclide where at least one of the more than one radionuclides is diagnostically active and at least one of the more than one radionuclide is therapeutically active. In some embodiments, the diagnostically active radionuclide is a radiohalogen and the therapeutically radionuclide is a radionuclide other than a radiohalogen. In other embodiments, the therapeutically and diagnostically active radionuclides are radiohalogens. In yet other embodiments, the therapeutically and diagnostically active radionuclides are radionuclides other than radiohalogens.

Alternatively, in some embodiments, more than one FAP-targeting compounds are used in a theragnostic method descried herein. In such embodiments, a diagnostically active FAP-targeting radioligand is used first to diagnose or image the disease or disorder and a therapeutically active FAP-targeting radioligand is subsequently used to treat the disease or disorder. For example, when the FAP-related disease or disorder is cancer, a diagnostically active FAP-targeting radioligand may initially be used to identify and localize the primary tumor mass, and to determine potential local and distant metastasis, and after imaging/diagnosis, a therapeutically active FAP-targeting radioligand (comprising a therapeutically active radionuclide) may be administered as described previously to treat the tumor.

When more than one FAP-targeting compounds are used in a theragnostic method, each FAP-targeting compound may be complexed to one or more than one radionuclide. In some embodiments, a diagnostically active FAP-targeting compound is complexed to one diagnostically active radionuclide. In some embodiments, a diagnostically active FAP-targeting compound is complexed to more than one diagnostically active radionuclide. In some embodiments, a therapeutically active FAP-targeting compound is complexed to one therapeutically active radionuclide. In some embodiments, a therapeutically active FAP-targeting compound is complexed to more than one therapeutically active radionuclide.

In some embodiments of the theragnostic method described herein, the diagnostic/imaging agent is a FAP-targeting imaging agent described in the art, such as, but not limited to, [¹⁸F]F-FAPI-74 or [¹⁸F]AlF-FAPI-74 (WO 2019/154886), [⁶⁸Ga]Ga-FAPI-46 (WO 2019/154886), [⁶⁸Ga]Ga-FAP-2286 (J. Nucl. Med. 2022, 63, 415-423), [^{99m}Tc]Tc-FL-L3 (Theranostics 2020, 10(13), 5778-5789), [⁶⁸Ga]Ga-MHLL1 (Theranostics 2021, 11(16), 7755-7766), [¹¹¹In]In-QCP02 (J. Med. Chem. 2021, 64(7), 4059-4070), [⁶⁸Ga]Ga-DOTA.SA.FAPi (EJNMMI Radiopharm. Chem. 2020, 5, 1-20), [⁶⁸Ga]Ga-RPS-309 (Mol. Imaging Biol. 2021, 23, 686-696), [⁶⁸Ga]Ga-FAPI-04 (Contrast Media Mol. Imaging 2022, 2022, 1-10), [⁶⁸Ga]Ga-FAP-21, [⁶⁸Ga]Ga-FAP-46 (J. Nucl. Med. 2019, 60(10), 1421-1429), [⁶⁸Ga]Ga-FAPI-02 (J. Nucl. Med. 2022, 63, 1844-1851), [^{99m}Tc]Tc-FAPI-34 (J. Nucl. Med. 2020, 61(10), 1507-1513), [¹⁸F]F-Glc-FAPI (Cancer Imaging 2023, 23, 1-15), [¹⁸F]AlF-NOTA-FAPI-04 (Eur. J. Nucl. Med. Mol. Imaging 2023, 50, 3425-3438), [⁶⁸Ga]Ga-PNT6555 (J. Nucl. Med 2024; 65:100-108), [⁶⁸Ga]Ga-OncoFAP or [⁶⁸Ga]Ga-OncoFAP-DOTAGA (Eur. J. Nucl. Med. Mol. Imaging 2022, 49, 1822-1832), [⁶⁸Ga]Ga-3BP-3940 (iScience, 2023, 26, 108541), [⁶⁸Ga]Ga-RPS-309 (Mol. Imaging Biol. 2021, 23(5), 686-696), or [⁶⁴Cu]Cu-RTX-1363S, and the therapeutic agent is a radiolabeled compound described herein (e.g., a radiolabeled compound of any one of Formulae (I), (Ia), (Ib), (Ic), (Id), and (I-i)).

In other embodiments of the theragnostic method described herein, the diagnostic/imaging agent is a radiolabeled compound described herein (e.g., a radiolabeled compound of any one of Formulae (I), (Ia), (Ib), (Ic), (Id), and (I-i)) and the therapeutic agent is a FAP-targeting therapeutic agent described in the art, such as, [¹⁷⁷Lu]Lu-PNT6555, [22′Ac]Ac- or [¹³¹I]I-CAM-FAP (J. Nucl. Med. 2022, 63, supplement 2, 2457), [¹⁷⁷Lu]Lu-OncoFAP, [¹⁷⁷Lu]Lu-3BP-3940, [¹⁷⁷Lu]Lu-RPS-309, radiolabeled-NM-05, [¹⁷⁷Lu]Lu-FAP-46, or [¹⁷⁷Lu]Lu-FAP-2286.

The dosage and administration schedule of a FAP-targeting compound used for theragnostic purposes may be the same as the dosage and administration schedules described above, e.g., a diagnostically active FAP-targeting compound used in a theragnostic method may be dosed and administered as described for a method of imaging and therapeutically active FAP-targeting compound used in a theragnostic method may be dosed and administered as described above for a method of treatment. A therapeutically and diagnostically active FAP-targeting compound may be dosed and administered according to either of the above methods. The dose and administration of a radioligand described herein, or a pharmaceutically acceptable salt or composition thereof to be administered to a subject may be determined by a person of skill in the art taking into account the disease or condition being diagnosed and treated, including its locale, and factors including age, weight, sex, and the like.

### Kits

The present disclosure also includes pharmaceutical kits useful, for example, in the imaging, diagnosis, or treatment of a FAP-related disease or disorder (such as, e.g., cancer) which include one or more containers containing a pharmaceutical composition comprising an effective amount (e.g., therapeutically effective amount) of a FAP-targeting ligand and/or radioligand of the disclosure. Such kits can further include one or more various kit components such as, for example, containers with one or more pharmaceutically acceptable carriers, additional containers (e.g., those suitable for handling radioactive waste), and/or instructions, inserts, or labels, indicating quantities of components to be administered, guidelines for administration, and/or guidelines for mixing the components.

The disclosure is further illustrated by the following examples and synthesis schemes, which are not to be construed as limiting this disclosure in scope or spirit. Those of skill in the art will readily recognize a variety of non-critical parameters which can be changed or modified to yield essentially the same or similar results. The compounds of the Examples have been found to selectively bind to human and/or mouse FAP according to at least one assay described herein.

### EXAMPLES

### 1. Abbreviations and General Procedures

### 1.1 Abbreviations

| | |
|---|---|
| AA | amino acid |
| AAZTA5 | 1,4-bis(carboxymethyl)-6-[bis(carboxymethyl)] |
| | amino-6-[pentanoic-acid]perhydro-1,4-diazepine |
| Ac | acetyl |
| ACN | acetonitrile |
| AcOEt | ethyl acetate |
| AcOH | acetic acid |
| Ac₂O | acetic anhydride |
| All | allyl |
| Alloc | allyloxycarbonyl |
| aq. | aqueous |
| Bal | β-alanine |
| Boc | tert-butoxycarbonyl |
| CPME | cyclopentyl methyl ether |
| DAD | diode-array detection |
| DBU | 1,8-diazabicyclo[5.4.0]undec-7-ene |
| DCE | 1,2-dichloroethane |
| DCM | dichloromethane |
| DIC | N,N′-diisopropylcarbodiimide |
| DIEA | N,N-diisopropylethylamine |
| DIPEA | N,N-diisopropylethylamine |
| DMA | N,N-dimethylacetamide |
| DMAP | 4-(dimethylamino)pyridine |
| DMF | N,N-dimethylformamide |
| DMSO | dimethylsulfoxide |
| DODT | 3,6-dioxa-1,8-octanedithiol |
| DOTA | 2,2′,2′′,2'′′′-(1,4,7,10-tetraazacyclododecane- |
| | 1,4,7,10-tetrayl)tetraacetic acid |
| DOTAGA | 2-(4,7,10-tris(carboxymethyl)-1,4,7,10- |
| | tetraazacyclododecan-1-yl)pentanedioic acid |
| eq. | equivalent(s) |
| ESI | electrospray ionization |
| EtOAc | ethyl acetate |
| Fmoc | 9-fluorenylmethyloxycarbonyl |
| Fmoc-OSu | 9-fluorenylmethyl-N-hydroxysuccinimide |
| g | gram |
| HATU | O-(7-azabenzotriazol-1-yl)-1,1,3,3-teramethyluronium |
| | hexafluorophosphate |
| HCTU | O-(1H-6-chlorobenzotriazol-1-yl)-1,1,3,3- |
| | tetramethyluronium hexafluorophosphate |
| HFIP | 1,1,1,3,3,3-hexafluoropropan-2-ol |
| HMPA | 2-(4-(hydroxymethyl)-3-methoxyphenoxy)acetic acid |
| HMPB | 4-(4-hydroxymethyl-3-methoxyphenoxy)butyric acid |
| HOBt | 1-hydroxybenzotriazole |
| HPLC | high-performance liquid chromatography |
| h | hour |
| IPA | isopropanol |
| L | liter |
| LC | liquid chromatography |
| LCMS | liquid chromatography mass spectrometry |
| M | molar |
| m/z | mass to charge ratio |
| MBHA | 4-methylbenzhydrylamine |
| MeOH | methanol |
| min | minute |
| Mol | mole |
| 3-MPA | 3-mercaptopropionic acid |
| MPE | 3-methyl-3-pentyl |
| MS | mass spectrometry |
| MTBE | tert-butyl methyl ether |
| NHS | N-hydroxysuccinimide |
| NIS | N-iodosuccinimide |
| NMP | N-methyl-2-pyrrolidone |
| NMR | nuclear magnetic resonance |
| NODAGA | 2-(4,7-bis(carboxymethyl)-1,4,7- |
| | triazonan-1-yl)pentanedioic acid |
| NOTA | 2,2′,2′′-(1,4,7-triazonane-1,4,7-triyl)triacetic acid |
| Oxyma | ethyl cyano(hydroxyimino)acetate |
| Pure ® | |
| PE | petroleum ether |
| PG | protecting group |
| Ph | phenyl |
| PS | polystyrene |
| PyAOP | (7-azabenzotriazol-1-yloxy)tripyrrolidino- |
| | phosphonium hexafluorophosphate |
| PyOxim | (ethyl cyano(hydroxyimino)acetato-O²)-tri- |
| | (1-pyrrolidinyl)-phosphonium hexafluorophosphate |
| QTof | quadrupole time of flight |
| RP | reversed-phase |
| rt | room temperature |
| Sar | sarcosine |
| sat. | saturated |
| SPPS | solid phase peptide synthesis |
| Cy5 | Cyanine5 |
| TBS | tert-butyldimethylsilyl |
| TBTU | 2-(1H-benzotriazole-1-yl)-1,1,3,3- |
| | tetramethylaminium tetrafluoroborate |
| tBu | tert-butyl |
| TEA | triethylamine |
| TFA | trifluoroacetic acid |
| THF | tetrahydrofuran |
| TIS | triisopropylsilane |
| TPTU | O-(1,2-dihydro-2-oxo-1-pyridyl)- |
| | N,N,N′,N′-tetramethyluronium tetrafluoroborate |
| tR | retention time |
| Trt | trityl, triphenylmethyl |
| UPLC | ultra-performance liquid chromatography |
| UV | ultraviolet |
| wt | weight |

### 1.2 General Procedures

Unless otherwise noted, reagents and solvents were used as received from commercial suppliers. Proton nuclear magnetic resonance (NMR) spectra were obtained on a Varian spectrometer at 400 MHz, a Bruker spectrometer at 400 MHz or 600 MHz. Chemical shifts are given in ppm (δ) and coupling constants, J, are reported in Hertz. Tetramethylsilane (TMS) or the solvent peak was used as an internal standard. Only the protons visible in 1H NMR were listed. Signals hidden under the solvent peaks were not included. The linear peptides were assembled by Fmoc-based solid phase peptide synthesis using typically peptide synthesizers, namely the Symphony X from Gyros Protein Technologies and the Liberty Prime from CEM. The syntheses were done at rt or with heating starting from H-Gly-Cl-Trt resin, H-Gly-Trt-Amide-MBHA resin or H-Gly-HMPB-Bal-MBHA resin (see section 7.1). Standard Fmoc-protected amino acids with appropriate protecting groups on side chains were used. Sometimes the special amino acid building blocks (see Table 7.1) were used. Amino acids were coupled using a variety of coupling reagents such as HATU, PyAOP, PyOxim, DIC/Oxyma Pure® and depending on the requirements a base such as DIEA. Fmoc was typically removed using 4-methylpiperidine, piperidine or pyrrolidine. Removal of the fully protected peptides from the resin was achieved with HFIP/DCM (e.g. 1/3) or a mixture of TFA/DCM (e.g. 1% TFA in DCM) or a mixture of TFA/DCE (e.g. 2% TFA in DCE). The linear peptides were cyclized directly or in some instances purified prior to cyclization. The cyclization was typically performed in DCM or DCE using HATU/2,6-lutidine. Other conditions used for cyclization were e.g. HATU/HOAt/2,6-lutidine, TBTU/HOBt/DIPEA or PyAOP/DIPEA in DCM or in ACN/DMF. Final deprotection was achieved by using e.g. the following mixtures: TFA/H₂O/DODT/TIS (87.5/5/5/2.5), TFA/H₂O/DODT/TIS (92.5/2.5/2.5/2.5), TFA/H₂O/3-MPA/TIS (92.5/2.5/2.5/2.5) or 95% aq. TFA/H₂O. Chelator coupling was performed using chelator reagents such as DOTA-NHS ester, NODAGA-NHS ester, NOTA-NHS ester, (R)-DOTAGA-anhydride, p-SCN-Bn-DOTA or tBu-protected AAZTA5, in the presence of a base (e.g., DIPEA), and if required coupling reagents such as HATU and HOAt. Labelling with natural lutetium, gallium or fluorine (via AlF) was performed in various buffers (e.g., ammonium acetate or sodium acetate buffers) at pH 3.5-5.0 at 80° C.-100° C., sometimes DMF was added.

The final products and intermediates were purified by preparative reversed-phase HPLC. The following columns were used:
XSelect Peptide CSH C18 OBD Prep Column, 130 Å, 5 μm, 30 mm×250 mm
XBridge BEH C18 OBD Prep Column, 130 Å, 5 μm, 30 mm×100 mm
XBridge BEH C18 OBD Prep Column, 130 Å, 5 μm, 30 mm×250 mm
XBridge BEH C18 OBD Prep Column, 130 Å, 5 μm, 50 mm×250 mm
SunFire C18 OBD Prep Column, 100 Å, 5 μm, 30 mm×100 mm
SunFire C18 OBD Prep Column, 100 Å, 5 μm, 50 mm×150 mm
Gemini C18, 110 Å, 5 μm, 30 mm×150 mm+Luna C18, 100 Å, 10 μm, 25 mm×120 mm

The following mobile phases were used:
Eluent A: H₂O+0.1% TFA and eluent B: ACN
Eluent A: H₂O+0.075% TFA and eluent B: ACN
Eluent A: H₂O+0.08% NH₄HCO₃ and eluent B: ACN

Gradients were designed based on the specific requirements of the separation problem. Pure products were lyophilized from ACN/H₂O and obtained, depending on the used eluents, as a free base or the corresponding trifluoroacetate. In certain cases, TFA salts were converted to sodium salts by the following method: TFA salt was dissolved in 8% aq. NaHCO₃ solution (pH 8.2) and the solution was stirred at rt for 15 min. The residue was purified by reversed-phase flash chromatography (Column: RediSep RF Gold, High Performance HP C18 Aq 30 g; Eluent A: H₂O and eluent B: ACN.

The products and intermediates were analyzed by the analytical methods described in Section 1.3 and Section 1.4.

### 1.3 Analytical Methods (Peptides)

### 1.3.1 Analytical Method P-1

Waters Acquity UPLC-MS system/QT of MS (ESI): Eluent A: Water+0.05% TFA; Eluent B: ACN+0.04% TFA; Column temperature: 80° C.; Flow: 0.5 mL/min; Column: Acquity UPLC CSH C18, 1.7 μm, 2.1×100 mm; Gradient: hold 5% B for 0.2 min; from 5 to 98% B in 9.2 min; hold 98% B for 0.4 min.

### 1.3.2 Analytical Method P-2

Waters Acquity UPLC-MS system/QT of MS (ESI): Eluent A: Water+0.05% TFA; Eluent B: ACN+0.04% TFA; Column temperature: 80° C.; Flow: 0.5 mL/min; Column: Acquity UPLC CSH C18, 1.7 μm, 2.1×100 mm; Gradient: hold 5% for 0.2 min; from 5 to 40% B in 8.8 min; from 40 to 98% B in 0.5 min.

### 1.3.3 Analytical Method P-3

Waters Acquity UPLC-MS system/QT of MS (ESI): Eluent A: Water+0.04% TFA; Eluent B: ACN+0.05% TFA; Column temperature: 80° C.; Flow: 1.0 mL/min; Column: Acquity UPLC CSH C18, 1.7 μm, 2.1×50 mm; Gradient: hold 2% for 0.2 min; from 2 to 98% B in 4.4 min; hold 98% B in 0.4 min.

### 1.3.4 Analytical Method P-4

Waters Acquity UPLC-MS system/MS (ESI): Eluent A: Water+0.1% FA; Eluent B: ACN+0.1% FA; Column temperature: 50° C.; Flow: 1.0 mL/min; Column: Acquity UPLC BEH C18, 1.7 μm, 2.1×50 mm; Gradient: 2 to 98% B in 8.0 min.

### 1.3.5 Analytical Method P-5

Agilent Infinity II 1260 HPLC&LCMS-G6125C (ESI): Eluent A: Water+0.1% TFA; Eluent B: ACN+0.075% TFA; Column temperature: 30° C.; Flow: 1.2 mL/min; Column: XBridge C18 3.5 μm 2.1×30 mm; Gradient: 10% to 80% B in 1.0 min; hold 95% B for 0.6 min; 95% to 10% B in 0.01 min; hold 10% B for 0.7 min.

### 1.3.6 Analytical Method P-6

Agilent Infinity 111290 HPLC&TOF (Dual AJS ESI): Eluent A: Water+0.1% TFA; Eluent B: ACN+0.075% TFA; Column temperature: 30° C.; Flow: 0.4 mL/min; Column: InfinityLab Poroshell 120 EC-C18-2.1×50 mm×1.9 μm; Gradient: 10% to 80% B in 1.0 min; hold 95% B for 0.6 min; 95% to 10%1B in 0.01 min; hold 10% B for 0.7 min.

### 1.3.7 Analytical Method P-7

Agilent Infinity II 1260 HPLC&LCMS-G6125C (ESI): Eluent A: Water+0.1% TFA; Eluent B: ACN+0.075% TFA; Column temperature: 30° C.; Flow: 1.2 mL/min; Column: XBridge C18 3.5 μm 2.1×30 mm; Gradient: 0% to 60%1B in 1.0 min; hold 95% B for 0.5 min; 95% to 0%1B in 0.01 min; hold 0% B for 0.8 min.

### 1.4 Analytical Methods (Building Blocks)

### 1.4.1 Analytical Method BB-1

Waters Acquity UPLC-MS system/MS (ESI): Eluent A: Water+0.05% formic acid+3.75 mM ammonium acetate; Eluent B: Isopropanol+0.05% formic acid; Column temperature: 80° C.; Flow: 0.4 mL/min; Column: ACQUITY UPLC® BEH C18, 1.7 μm, 2.1×100 mm; Gradient: hold 5% B for 0.5 min; 5% to 60%1B in 8.4 min; 60 to 98% B in 1.0 min; hold 98% B for 0.4 min.

### 1.4.2 Analytical Method BB-2

SHIMADZU LCMS-2020 system/MS (ESI): Eluent A: Water+0.0375% TFA; Eluent B: ACN+0.01875% TFA; Column temperature: 50° C.; Flow: 1.5 mL/min; Column: HALO C18, 5.0 μm, 3.0×30 mm; Gradient: 5% to 95%1B in 0.5 min; hold 95% B for 0.3 min; 95% to 5%1B in 0.01 min; hold 5% B for 0.24 min.

### 1.4.3 Analytical Method BB-3

SHIMADZU LCMS-2020 system/MS (ESI): Eluent A: Water+0.025% NH₄OH; Eluent B: ACN; Column temperature: 40° C.; Flow: 1.5 mL/min; Column: Kinetex EVO C18, 5.0 μm, 2.1×30 mm; Gradient: 5% to 95%1B in 0.8 min; hold 95% B for 0.4 min; 95% to 5%1B in 0.01 min; hold 5% B for 0.34 min.

### 1.4.4 Analytical Method BB-4

SHIMADZU LCMS-2020 system/MS (ESI): Eluent A: Water+0.0375% TFA; Eluent B: ACN+0.01875% TFA; Column temperature: 50° C.; Flow: 1.5 mL/min; Column: HALO C18, 5.0 μm, 3.0×30 mm; Gradient: 50% to 100%1B in 0.5 min; hold 95% B for 0.3 min; 95% to 5%1B in 0.01 min; hold 5% B for 0.24 min.

### 1.4.5 Analytical Method BB-5

Waters Acquity UPLC H-Class/MS SQD2 (ESI): Eluent A: Water+0.025% TFA; Eluent B: ACN+0.025% TFA; Column temperature: 60° C.; Flow: 0.6 mL/min; Column: Kinetex EVO C18, 1.7 μm, 2.1×100 mm; Gradient: from 5 to 95% B in 2.1 min, hold 95% for 0.74 min, hold 5% for 0.66 min.

### 1.4.6 Analytical Method BB-6

Waters Acquity UPLC-MS system/MS (ESI): Eluent A: Water+0.05% formic acid+3.75 mM ammonium acetate; Eluent B: isopropanol+0.05% formic acid; Column temperature: 80° C.; Flow: 1.0 mL/min; Column: CORTECS C18+, 2.1×50 mm, 2.7 μm; Gradient: 5% to 50% B in 1.4 min; 50 to 98% B in 0.3 min; hold 98% B for 0.1 min.

### 2. Assembly of Cyclic Peptides (“A” Examples)

### 2.1 Examples Synthesized Starting from A¹ (i.e., P1 is A¹-Resin)

### 2.1.1 Synthesis of 2-((9S,12S,15S,18S,24S,27S,32aR)-18-((1H-indol-3-yl)methyl)-12-(4-((2-(2-(2-aminoethoxy)ethoxy)acetamido)methyl)benzyl)-9-benzyl-24-(4-(4-(carboxymethyl)piperazine-1-carboxamido)butyl)-8-methyl-1,4,7,10,13,16,19,22,25,28-decaoxo-15-(2-propoxyethyl)dotriacontahydropyrrolo[1,2-a][1,4,7,10,13,16,19,22,25,28]decaazacyclotriacontin-27-yl)acetic acid TFA salt (Example A2)

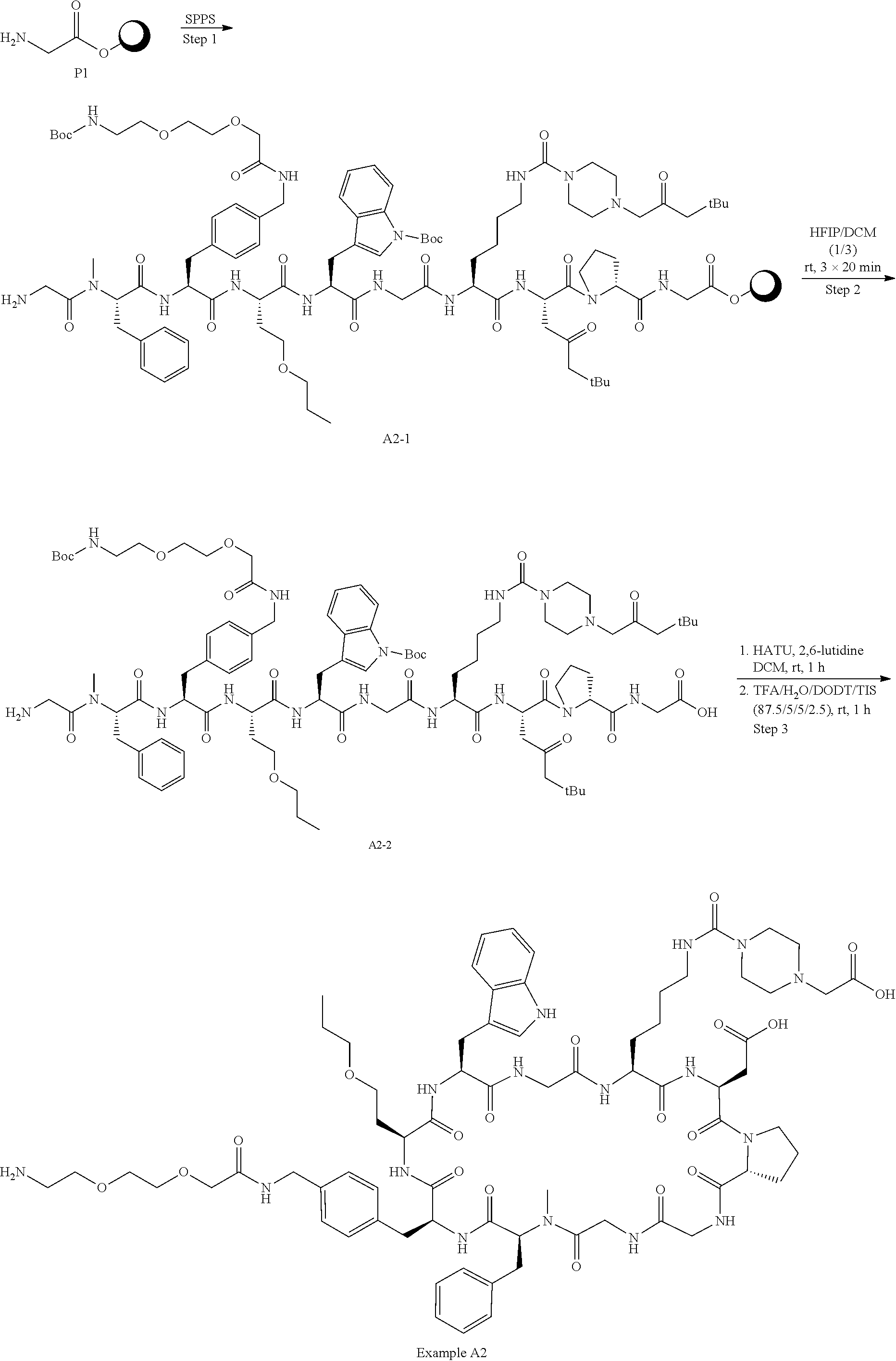

### Scheme 2.1.1. Synthesis of Example A2

### Step 1. A2-1

The assembly of the linear peptide was done on a Symphony X from Gyros Protein Technologies at rt using preloaded resin P1 (3.719 g, 1.80 mmol, 0.484 mmol/g) in 6 batches of 0.30 mmol. The amino acids were coupled with a set of different methods.

### Method A

Fmoc-amino acid (0.2 M solution in NMP, 3 eq.), HATU (0.3 M solution in NMP, 3 eq.), DIEA (0.33 M solution in NMP, 3.3 eq.), addition by synthesizer.

### Method B

Fmoc-amino acid (0.2 M solution in NMP, 3 eq.), PyOxim (0.3 M solution in NMP, 3 eq.), DIEA (0.33 M solution in NMP, 6.6 eq.), addition by synthesizer.

### Method C (Manual Addition)

Fmoc-amino acid (1.25 eq.) and PyAOP (2.5 eq.) were dissolved in NMP (5 mL) and DIEA (3.125 eq.) was added. After a pre-activation time of 5 min the solution was added to the resin. The Fmoc removal was performed using a solution of 4-methylpiperidine/DMA (1/4). Capping was performed using Ac₂O/pyridine/DMA (1/1/8). After the assembly of the linear peptide the resin A2-1 was washed with DMA (5×) and DCM (5×). The coupling methods and times are summarized in Table 2.1.

TABLE 2.1

| Synthesis | Amino acid | | | |
|---|---|---|---|---|
| cycle | residue* | Amino acid | Time | Method |
| 1 | A¹⁰ | Fmoc-D-Pro-OH | 2 × 2 h | A |
| 2 | A⁹ | Fmoc-Asp(tBu)-OH | 2 × 3 h | B |
| 3 | A⁸ | BB41 | 1 × 3 h | C |
| 4 | A⁷ | Fmoc-Gly-OH | 2 × 2 h | A |
| 5 | A⁶ | Fmoc-Trp(Boc)-OH | 3 × 1 h | A |
| 6 | A⁵ | BB21 | 1 × 3 h | C |
| 7 | A⁴ | BB40 | 1 × 3 h | C |
| 8 | A³ | Fmoc-N-Me-Phe-OH | 2 × 2 h | A |
| 9 | A² | Fmoc-Gly-OH | 2 × 3 h | B |

*Refers to the corresponding amino acid residue of cyclic peptide, P, as found, e.g., in Formulae (I), (Ia), (Ib), (Ic), (Id), (I-i), or in the Table of Example III.

### Step 2. A2-2

To the resin A2-1 (calculated with 1.80 mmol) was added HFIP/DCM (1/3) (60 mL). The resin was shaken at rt for 20 min. The cleavage solution was filtered off and the procedure was repeated two times. The resin was washed with HFIP/DCM (1/3) (20 mL). The combined cleavage and washing solutions were concentrated to dryness in vacuo. The residue was treated with toluene and the resulting mixture was concentrated to dryness in vacuo. The residue was dried in high vacuum at 40° C. and A2-2 was obtained as an off-white solid. LCMS method P-1, t_{R}=4.70 min, [M+H]⁺=1824.0.

### Step 3. Example A2

Step 3-1: To a clear solution of A2-2 (calculated with 1.80 mmol) in DCM (1800 mL) at rt were added HATU (1.37 g, 2 eq., 3.6 mmol), followed by 2,6-lutidine (4.82 g, 5.21 mL, 25 eq., 45 mmol). The reaction mixture was stirred at rt for 1 h, then concentrated to dryness in vacuo. LCMS method P-1, t_{R}=5.91 min, [M+H]⁺=1806.0.

Step 3-2: The residue from Step 3-1 was dissolved in TFA/H₂O/DODT/TIS (87.5/5/5/2.5) (100 mL) and the reaction mixture was stirred at rt for 1 h. The clear solution was then poured onto cold methyl tert-butyl ether/heptanes (1/1) (600 mL), giving a precipitate. The suspension was centrifuged and the solvent was decanted. The residue was washed with cold methyl tert-butyl ether/heptanes (1/1) (2×240 mL), then dried in high vacuum at 40° C. for 1 h. The deprotection of Trp was incomplete, the precipitate was therefore dissolved in ACN/H₂O (1/1, 60 mL) and stirred for 5.5 h at rt, then lyophilized. The product was isolated by preparative HPLC (Column: XBridge BEH C18 OBD Prep Column, 130 Å, 5 μm, 50 mm×250 mm; Eluent A: H₂O+0.1% TFA and eluent B: ACN). Pure fractions were combined and lyophilized to afford Example A2 (1.041 g, 0.59 mmol, 33% yield, TFA salt) as a white solid. LCMS method P-1, t_{R}=3.13 min, [M+H]⁺=1493.8.

The compounds in Table 2.2 were synthesized in analogy to Example A2.

TABLE 2.2
Examples synthesized in analogy to Example A2 (Scheme 2.1.1)

| Example No. | Structure/Example Name | LCMS |
|---|---|---|
| A1 | 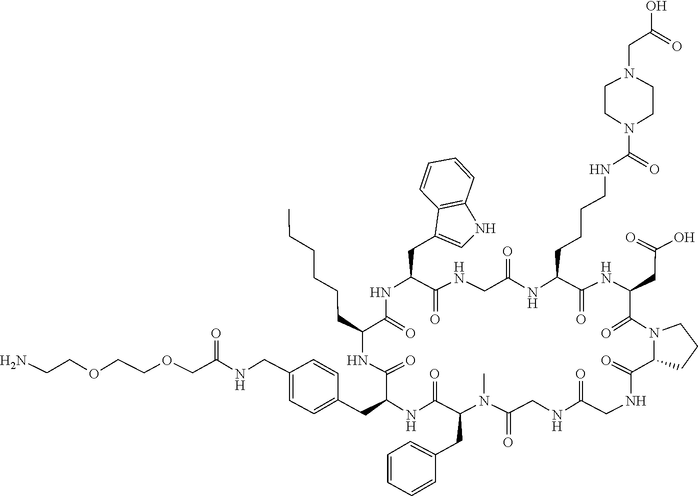 [SMILES: CCCCCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.59 min [M + H]⁺ = 1491.9 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-24-(4-(4- | |
| | (carboxymethyl)piperazine-1-carboxamido)butyl)-15-hexyl-8-methyl- | |
| | 1,4,7,10,13,16,19,22,25,28-decaoxodotriacontahydropyrrolo[1,2- | |
| | a][1,4,7,10,13,16,19,22,25,28]decaazacyclotriacontin-27-yl)acetic acid TFA salt | |
| A50 | 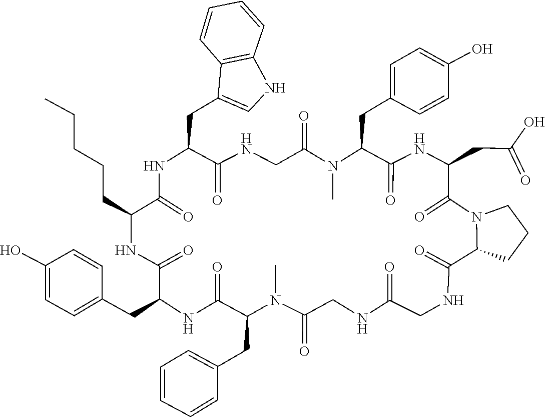 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)N(C)C(=O)CNC(=O)[C@H](Cc2c[nH]c3ccccc23)NC1=O] | Method P-1 t_{R} = 4.82 min [M + H]⁺ = 1198.6 |
| | 2-((9S,12S,15S,18S,24S,27S,32aR)-18-((1H-indol-3-yl)methyl)-9-benzyl-12,24- | |
| | bis(4-hydroxybenzyl)-8,23-dimethyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A75 | 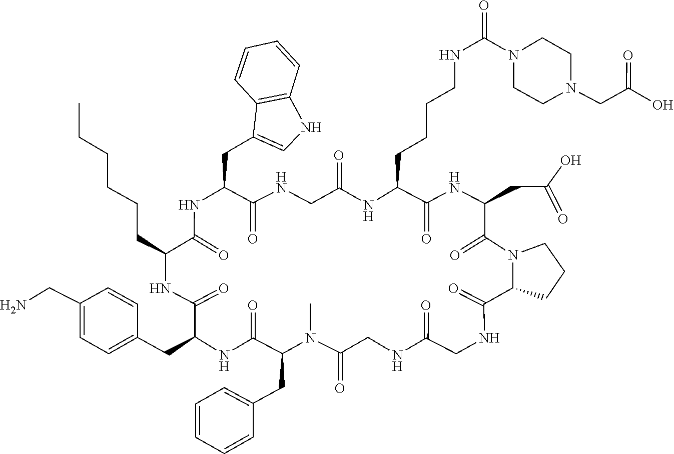 [SMILES: CCCCCC[C@@H]1NC(=O)[C@H](Cc2ccc(CN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.43 min [M + H]⁺ = 1346.7 |
| | 2-((9S,12S,15S,18S,24S,27S,32aR)-18-((1H-indol-3-yl)methyl)-12-(4- | |
| | (aminomethyl)benzyl)-9-benzyl-24-(4-(4-(carboxymethyl)piperazine-1- | |
| | carboxamido)butyl)-15-hexyl-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-27-yl)acetic acid TFA salt | |
| A76 | 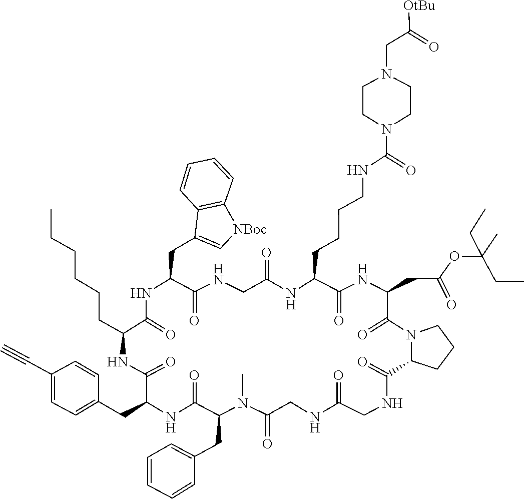 [SMILES: C#Cc1ccc(C[C@@H]2NC(=O)[C@H](Cc3ccccc3)N(C)C(=O)CNC(=O)CNC(=O)[C@H]3CCCN3C(=O)[C@H](CC(=O)OC(C)(CC)CC)NC(=O)[C@H](CCCCNC(=O)N3CCN(CC(=O)OC(C)(C)C)CC3)NC(=O)CNC(=O)[C@H](Cc3cn(C(=O)OC(C)(C)C)c4ccccc34)NC(=O)[C@H](CCCCCC)NC2=O)cc1] | Method P-3 t_{R} = 2.91 min [M + H]⁺ = 1581.8 |

tert-butyl 3-(((9S,12S,15S,18S,24S,27S,32aR)-9-benzyl-24-(4-(4-(2-(tert-butoxy)-2-
oxoethyl)piperazine-1-carboxamido)butyl)-12-(4-ethynylbenzyl)-15-hexyl-8-methyl-
27-(2-((3-methylpentan-3-yl)oxy)-2-oxoethyl)-1,4,7,10,13,16,19,22,25,28-
decaoxodotriacontahydropyrrolo[1,2-
a][1,4,7,10,13,16,19,22,25,28]decaazacyclotriacontin-18-yl)methyl)-1H-indole-1-
carboxylate TFA salt

### 2.1.2 Synthesis of 2-((9S,12S,15S,18S,24S,27S,32aR)-18-((1H-indol-3-yl)methyl)-9-benzyl-24-(4-(4-(carboxymethyl)piperazine-1-carboxamido)butyl)-12-(4-hydroxybenzyl)-8,23-dimethyl-1,4,7,10,13,16,19,22,25,28-decaoxo-15-pentyldotriacontahydropyrrolo[1,2-a][1,4,7,10,13,16,19,22,25,28]decaazacyclotriacontin-27-yl)acetic acid TFA salt (Example A3)

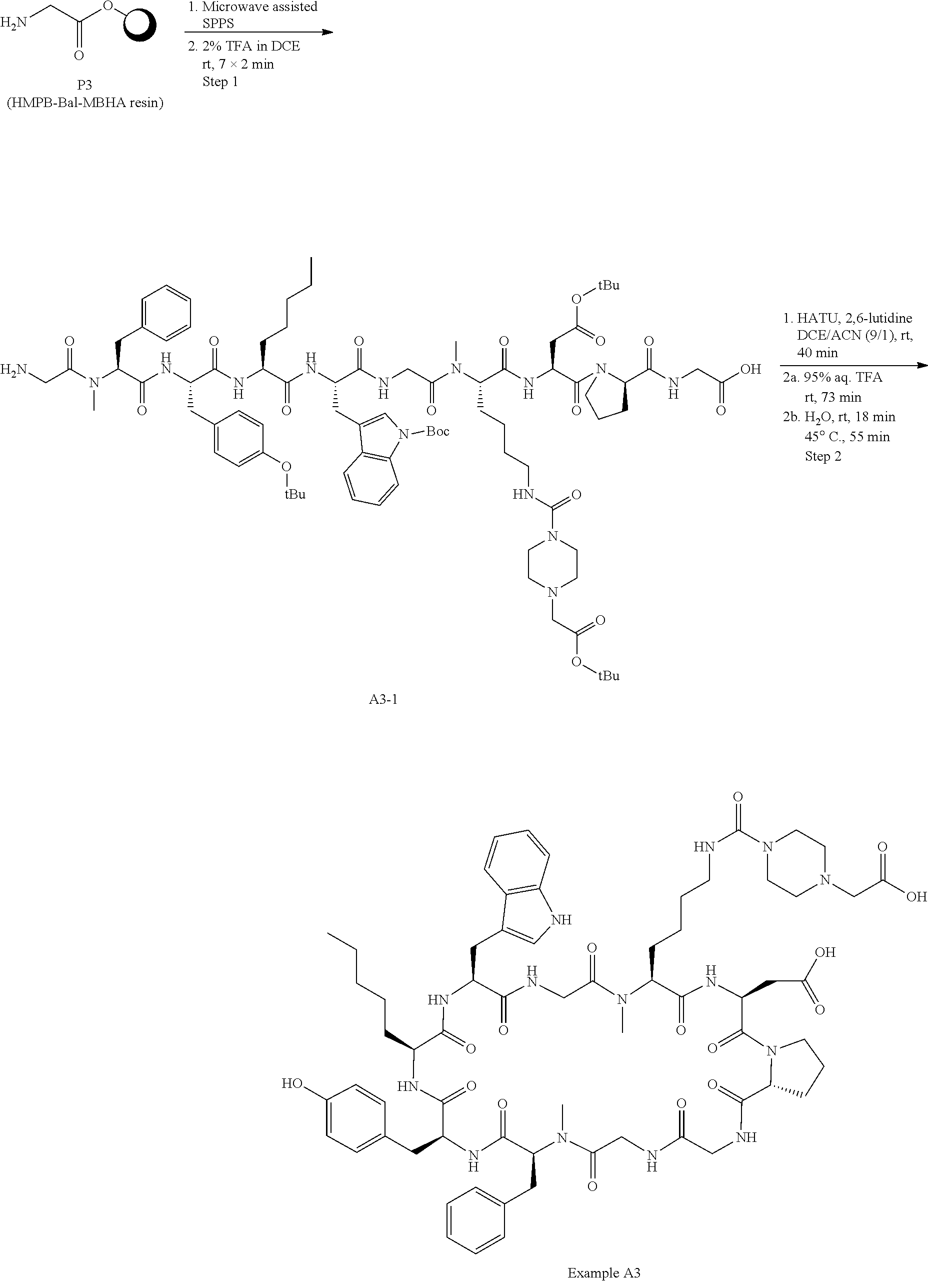

### Scheme 1.1.2. Synthesis of Example A3

### Step 1. A3-1

Step 1-1: The assembly of the linear peptide was done on a CEM Liberty Prime 1.0 from CEM using preloaded resin P3 (323 mg, 251 μmol, 0.776 mmol/g). The amino acids were coupled with a set of different methods.

### Method A

Fmoc-amino acid (0.2 M solution in 0.19 M OxymaPure in NMP, 2.39 eq.), DIC (0.64 M solution in NMP, 3.37 eq.), NMP (2 mL), addition by synthesizer, 2×10 min at 80° C.

### Method B

Fmoc-amino acid (0.2 M solution in 0.19 M OxymaPure in NMP, 1.52 eq.), DIC (0.64 M solution in NMP, 2.30 eq.), NMP (1.5 mL), addition by synthesizer, 10 min at 80° C.

### Method C

Fmoc-amino acid (0.2 M solution in 0.19 M OxymaPure in NMP, 2.39 eq.), DIC (0.64 M solution in NMP, 3.37 eq.), NMP (2 mL), addition by synthesizer, 6 min at 80° C.

### Method D

Fmoc-amino acid (0.2 M solution in 0.19 M OxymaPure in NMP, 1.52 eq.), DIC (0.64 M solution in NMP, 2.30 eq.), NMP (2 mL), addition by synthesizer, 4 min at 80° C. The Fmoc removal was performed using 10% 4-methylpiperidine in 0.3 M OxymaPure in NMP (4 mL) at 50° C. for 2×0.8 min.

Final Fmoc deprotection was performed using 10% 4-methylpiperidine in 0.3 M OxymaPure in NMP at 50° C. for 2×1 min.

Capping was performed using Ac₂O/pyridine/DMA (1/1/8) at 60° C. for 45 s. After the assembly of the linear peptide the resin was washed with DMA (5×) and DCM (5×). The coupling methods and times are summarized in Table 2.3.

TABLE 2.3

| Synthesis | Amino acid | | | |
|---|---|---|---|---|
| cycle | residue* | Amino acid | Time | Method |
| 1 | A¹⁰ | Fmoc-D-Pro-OH | 1 × 4 min, 80° C. | D |
| 2 | A⁹ | Fmoc-Asp(tBu)-OH | 1 × 6 min, 80° C. | C |
| 3 | A⁸ | BB42 | 1 × 10 min, 80° C. | B |
| 4 | A⁷ | Fmoc-Gly-OH | 2 × 10 min, 80° C. | A |
| 5 | A⁶ | Fmoc-Trp(Boc)-OH | 1 × 6 min, 80° C. | C |
| 6 | A⁵ | Fmoc-Ahp-OH | 1 × 6 min, 80° C. | C |
| 7 | A⁴ | Fmoc-Tyr(tBu)-OH | 1 × 6 min, 80° C. | C |
| 8 | A³ | Fmoc-N-Me-Phe-OH | 1 × 10 min, 80° C. | B |
| 9 | A² | Fmoc-Gly-OH | 2 × 10 min, 80° C. | A |

*Refers to the corresponding amino acid residue of cyclic peptide, P, as found, e.g., in Formulae (I), (Ia), (Ib), (Ic), (Id), or in the Table of Example III.

Step 1-2: To the resin was added 2% TFA in DCE (4 mL). The resin was shaken at rt for 2 min. The cleavage solution was filtered off and the procedure was repeated 6 times. The combined cleavage solutions were concentrated to dryness in vacuo. The residue was lyophilized from 80% aq. dioxane (6 mL) to afford the crude peptide. The peptide was isolated by preparative reversed-phase HPLC (Column: SunFire C18 OBD Prep Column, 100 Å, 5 μm, 50 mm×150 mm; Eluent A: H₂O+0.1% TFA and eluent B: ACN). Pure fractions were combined and lyophilized. TFA was removed using an Agilent PL HCO3 MP 500 mg/6 mL SPE column and mixtures of ACN/H₂O as eluents. The fractions were concentrated to dryness in vacuo and the residue was lyophilized from 80% aq. dioxane to afford A3-1 (65 mg, 40.1 μmol, 16% yield) as a white solid. LCMS method P-1, t_{R}=5.15 min, [M+H]⁺=1619.9.

### Step 2. Example A3

Step 2-1: A3-1 (63.5 mg, 39.2 μmol) and 2,6-lutidine (12.6 mg, 13.7 μl, 3 eq., 118 μmol) were dissolved in DCE/ACN (9/1) (3.92 mL). This solution was added to a freshly prepared solution of HATU (28.3 mg, 1.9 eq., 74.5 μmol) and 2,6-lutidine (16.0 mg, 17.3 μl, 3.8 eq., 149 μmol) in DCE/ACN (9/1) (35 mL). The reaction was stirred at rt for 40 min, then concentrated to dryness in vacuo.

Step 2-2: The crude peptide from Step 2-1 was dissolved in 95% aq. TFA (1.5 mL) and the reaction was stirred at rt for 73 min. H₂O (2.5 mL) was added and the reaction was stirred at rt for 18 min, then at 45° C. for 55 min. The product was isolated by preparative reversed-phase HPLC (Column: SunFire C18 OBD Prep Column, 100 Å, 5 μm, 50 mm×150 mm; Eluent A: H₂O+0.1% TFA and eluent B: ACN). Pure fractions were combined and lyophilized to afford Example A3 (19.7 mg, 13.4 μmol, 34% yield) as a white solid. LCMS method P-1, t_{R}=4.05 min, [M+H]⁺=1333.7.

The compounds in Table 2.4 were synthesized in analogy to Example A3.

TABLE 2.4
Example A74 was synthesized in analogy to Example A3 (Scheme 2.1.2)

| Example | | |
|---|---|---|
| No. | Structure/Example Name | LCMS |
| A74 | 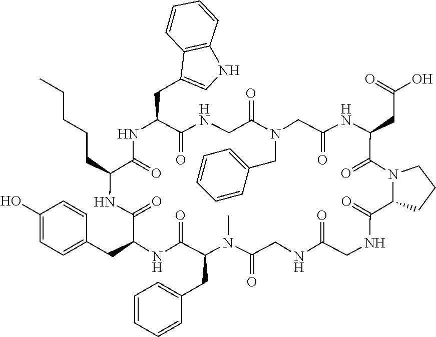 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)CN(Cc2ccccc2)C(=O)CNC(=O)[C@H](Cc2c[nH]c3ccccc23)NC1=O] | Method P-1 t_{R} = 5.17 min [M + H]⁺ = 1168.6 |
| | 2-((9S,12S,15S,18S,27S,32aR)-18-((1H-indol-3-yl)methyl)-9,23-dibenzyl-12-(4- | |
| | hydroxybenzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |

### 2.2 Examples Synthesized Starting from A² (i.e., P1 is A²-Resin)

### 2.2.1 Synthesis of 2-((9S,12S,15S,18S,24S,27S,32aR)-18-((1H-indol-3-yl)methyl)-12-(4-((2-(2-(2-aminoethoxy)ethoxy)acetamido)methyl)benzyl)-9-benzyl-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-27-yl)acetic acid TFA salt (Example A1)

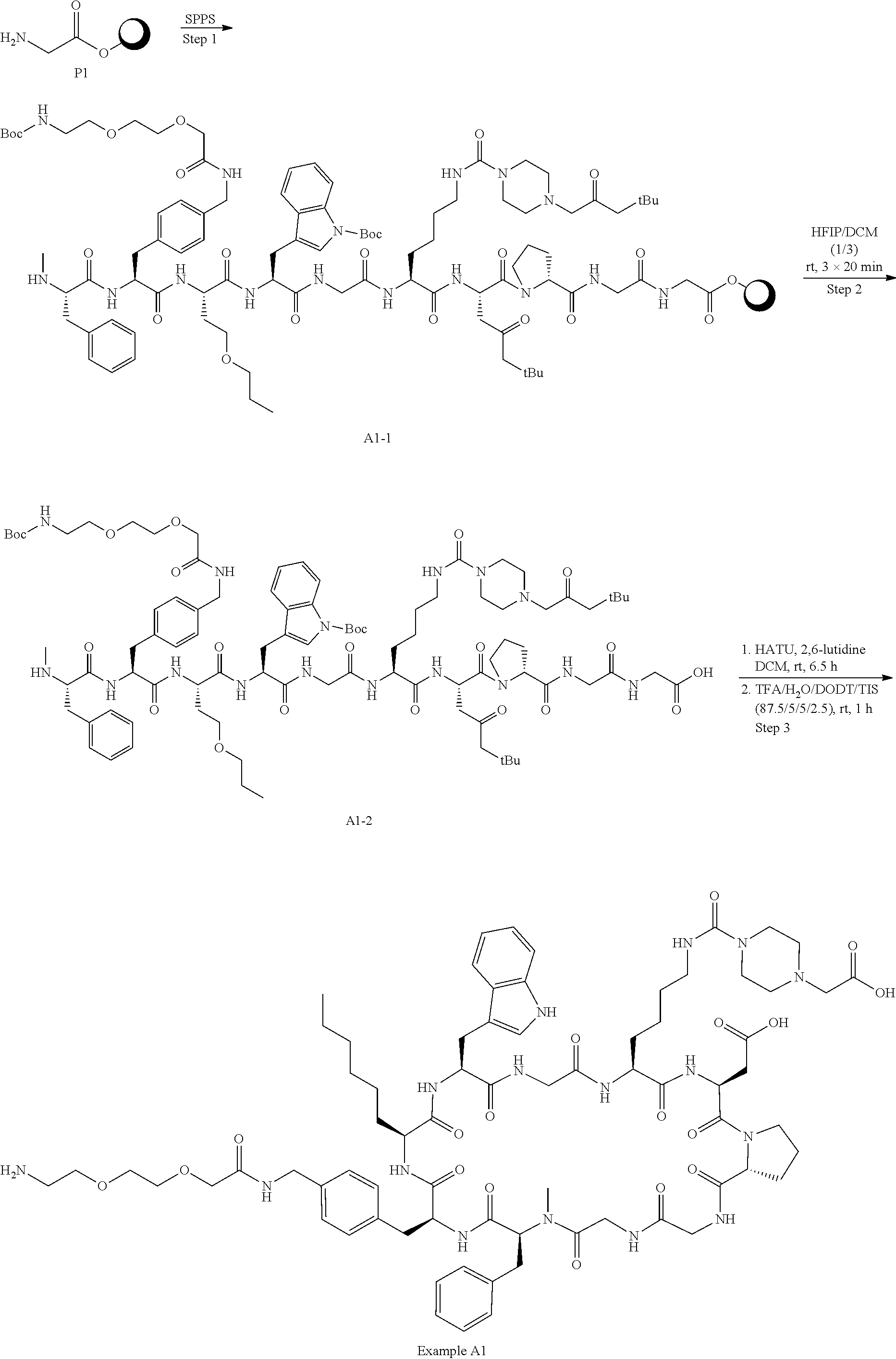

### Scheme 2.2.1. Synthesis of Example A1

### Step 1. A1-1

The assembly of the linear peptide was done on a Symphony X from Gyros Protein Technologies at rt using preloaded resin P1 (192.3 mg, 100 μmol, 0.52 mmol/g). The amino acids were coupled with a set of different methods.

### Method A

Fmoc-amino acid (0.2 M solution in NMP, 3 eq.), HATU (0.3 M solution in NMP, 3 eq.), DIEA (0.33 M solution in NMP, 3.3 eq), addition by synthesizer.

### Method B

Fmoc-amino acid (0.2 M solution in NMP, 3 eq.), PyOxim (0.3 M solution in NMP, 3 eq.), DIEA (0.33 M solution in NMP, 6.6 eq.), addition by synthesizer.

### Method C (Manual Addition)

Fmoc-amino acid (3 eq.) and HATU (3 eq.) were dissolved in NMP (3 mL) and DIEA (3.3 eq) was added. After a preactivation time of 5 min the solution was added to the resin.

### Method D (Manual Addition)

Fmoc-amino acid (1.5 eq.) and HATU (1.5 eq.) were dissolved in NMP (1 mL) and DIEA (1.65 eq.) was added. After a preactivation time of 5 min the solution was added to the resin.

The Fmoc removal was performed using a solution of 4-methylpiperidine/DMA (1/4). Capping was performed using Ac₂O/pyridine/DMA (1/1/8). After assembly of the linear peptide A1-1 the resin was washed with DMA (5×) and DCM (5×). The coupling methods and times are summarized in Table 2.5.

TABLE 2.5

| Synthesis | Amino acid | | | |
|---|---|---|---|---|
| cycle | residue* | Amino acid | Time | Method |
| 1 | A¹ | Fmoc-Gly-OH | 2 × 1 h | A |
| 2 | A¹⁰ | Fmoc-D-Pro-OH | 2 × 1 h | A |
| 3 | A⁹ | Fmoc-Asp(tBu)-OH | 2 × 3 h | B |
| 4 | A⁸ | BB41 | 1 × 3 h | C |
| 5 | A⁷ | Fmoc-Gly-OH | 2 × 1 h | A |
| 6 | A⁶ | Fmoc-Trp(Boc)-OH | 2 × 1 h | A |
| 7 | A⁵ | Fmoc-Aoc-OH | 1 × 3 h | C |
| 8 | A⁴ | BB40 | 1 × 3 h | D |
| 9 | A³ | Fmoc-N-Me-Phe-OH | 2 × 1 h | A |

*Refers to the corresponding amino acid residue of cyclic peptide, P, as found, e.g., in Formulae (I), (Ia), (Ib), (Ic), (Id), (I-i), or in the Table of Example III.

### Step 2. A1-2

To A1-1 (calculated with 100 μmol) was added HFIP/DCM (1/3) (3 mL). The resin was shaken at rt for 20 min. The cleavage solution was filtered off and the procedure was repeated two times. The resin was washed with HFIP/DCM (1/3) (1 mL). The combined cleavage and washing solutions were concentrated to dryness in vacuo. The residue was treated with toluene and the resulting mixture was concentrated to dryness in vacuo. The residue was dried in high vacuum at 40° C. and A1-2 was obtained as an off-white solid. LCMS method P-1, t_{R}=4.97 min, [M+H]⁺=1822.0.

### Step 3. Example A1

Step 3-1: To a clear solution of the linear peptide A1-2 (calculated with 100 μmol) in DCM (100 mL) at rt were added HATU (76.1 mg, 2 eq., 200 μmol), followed by 2,6-lutidine (268 mg, 290 μL, 25 eq., 2.50 mmol). The reaction mixture was stirred at rt for 6.5 h, then concentrated to dryness in vacuo. LCMS P-1, t_{R}=6.18 min, [M+H]⁺=1804.0.

Step 3-2: The residue was dissolved in TFA/H₂O/DODT/TIS (87.5/5/5/2.5) (7 mL) and the reaction mixture was stirred at rt for 1 h. The clear solution was then poured onto cold methyl tert-butyl ether/heptanes (1/1) (40 mL), giving a precipitate. The suspension was centrifuged and the solvent was decanted. The residue was washed with cold methyl tert-butyl ether/heptanes (1/1) (2×15 mL), then dried in high vacuum at 40° C. for 30 min. The product was isolated by preparative HPLC (Column: XSelect Peptide CSH C18 OBD Prep Column, 130 Å, 5 μm, 30 mm×250 mm; Eluent A: H₂O+0.1% TFA and eluent B: ACN). Pure fractions were combined and lyophilized to afford Example A1 (72.2 mg, 40 μmol, 40% yield, TFA salt) as a white solid. LCMS method P-1, t_{R}=3.67 min, [M+H]⁺=1491.8.

The examples in Table 2.6 were synthesized in analogy to Example A1.

TABLE 2.6
Examples synthesized in analogy to Example A1 (Scheme 2.2.1)

| Ex- | | |
|---|---|---|
| ample | | |
| No. | Structure/Example Name | LCMS |
| A4-1 | 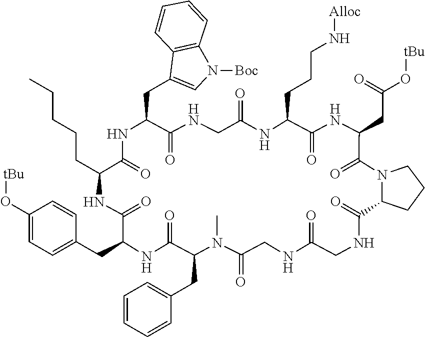 | Method P-1 t_{R} = 7.65 min [M + H]⁺ = 1431.8 |
| | tert-butyl 3-(((9S,12S,15S,18S,24S,27S,32aR)-24-(3- | |
| | (((allyloxy)carbonyl)amino)propyl)-9-benzyl-27-(2-(tert-butoxy)-2-oxoethyl)-12-(4- | |
| | (tert-butoxy)benzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-18-yl)methyl)-1H-indole-1- | |
| | carboxylate | |
| A5-1 | 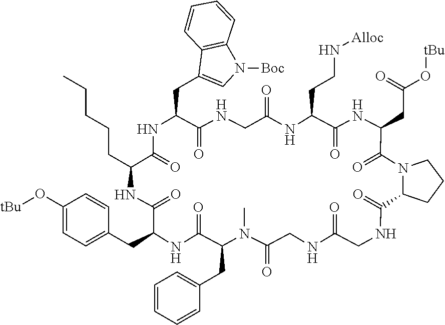 | Method P-1 t_{R} = 7.64 min [M + H]⁺ = 1417.8 |
| | tert-butyl 3-(((9S,12S,15S,18S,24S,27S,32aR)-24-(2- | |
| | (((allyloxy)carbonyl)amino)ethyl)-9-benzyl-27-(2-(tert-butoxy)-2-oxoethyl)-12-(4- | |
| | (tert-butoxy)benzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-18-yl)methyl)-1H-indole-1- | |
| | carboxylate | |
| A6 | 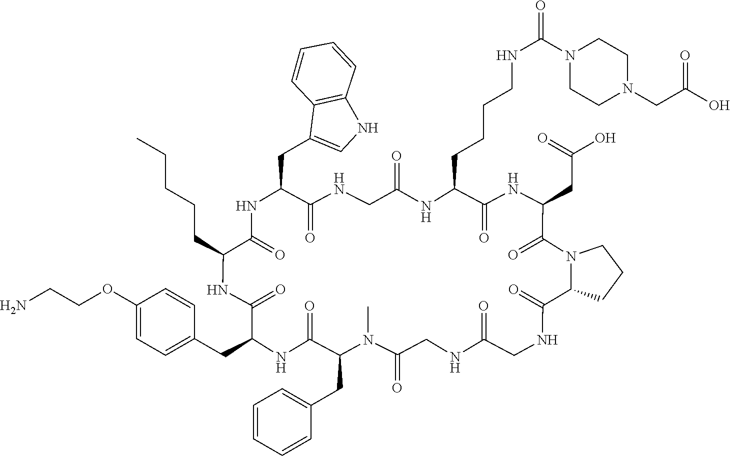 [SMILES: CCCCC[C@@H]1NC(=O)[C@H](Cc2ccc(OCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-2 t_{R} = 6.15 min [M + H]⁺ = 1362.7 |
| | 2-((9S,12S,15S,18S,24S,27S,32aR)-18-((1H-indol-3-yl)methyl)-12-(4-(2- | |
| | aminoethoxy)benzyl)-9-benzyl-24-(4-(4-(carboxymethyl)piperazine-1- | |
| | carboxamido)butyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid TFA salt | |
| A7 | 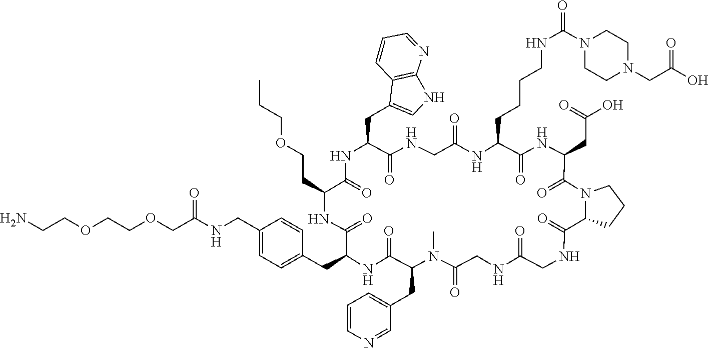 [SMILES: CCCOCC[C@@H]1NC(=O)[C@H](Cc2ccc(CNC(=O)COCCOCCN)cc2)NC(=O)[C@H](Cc2cccnc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 2.46 min [M + H]⁺ = 1495.7 |
| | 2-((9S,12S,15S,18S,24S,27S,32aR)-18-((1H-pyrrolo[2,3-b]pyridin-3-yl)methyl)-12-(4- | |
| | ((2-(2-(2-aminoethoxy)ethoxy)acetamido)methyl)benzyl)-24-(4-(4- | |
| | (carboxymethyl)piperazine-1-carboxamido)butyl)-8-methyl- | |
| | 1,4,7,10,13,16,19,22,25,28-decaoxo-15-(2-propoxyethyl)-9-(pyridin-3- | |
| | ylmethyl)dotriacontahydropyrrolo[1,2- | |
| | a][1,4,7,10,13,16,19,22,25,28]decaazacyclotriacontin-27-yl)acetic acid TFA salt | |
| A8 | 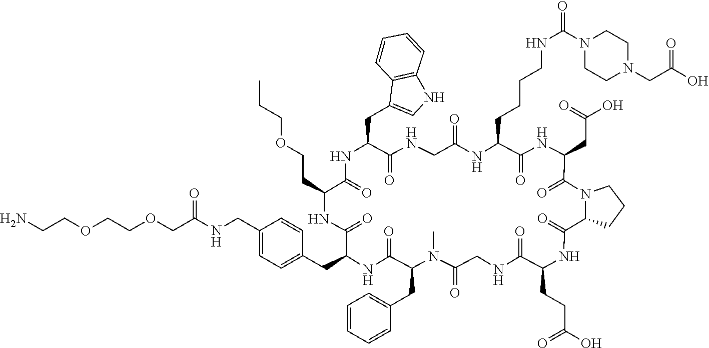 [SMILES: CCCOCC[C@@H]1NC(=O)[C@H](Cc2ccc(CNC(=O)COCCOCCN)cc2)NC(=O)[C@H](Cc2ccccc2)N(C)C(=O)CNC(=O)[C@H](CCC(=O)O)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.07 min [M + H]⁺ = 1565.8 |
| | 3-((3S,9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-27-(carboxymethyl)-24-(4- | |
| | (4-(carboxymethyl)piperazine-1-carboxamido)butyl)-8-methyl- | |
| | 1,4,7,10,13,16,19,22,25,28-decaoxo-15-(2-propoxyethyl)dotriacontahydropyrrolo[1,2- | |
| | a][1,4,7,10,13,16,19,22,25,28]decaazacyclotriacontin-3-yl)propanoic acid TFA salt | |
| A9 | 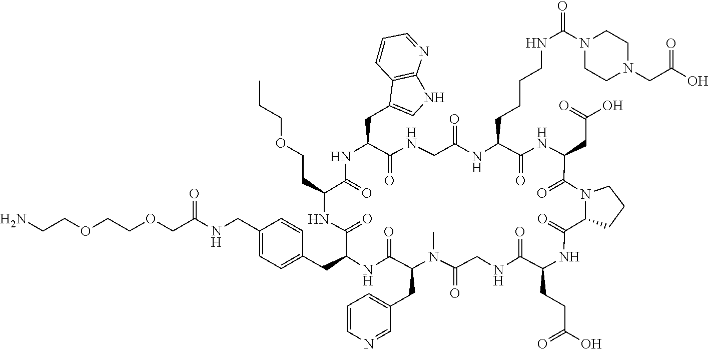 [SMILES: CCCOCC[C@@H]1NC(=O)[C@H](Cc2ccc(CNC(=O)COCCOCCN)cc2)NC(=O)[C@H](Cc2cccnc2)N(C)C(=O)CNC(=O)[C@H](CCC(=O)O)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 2.46 min [M + H]⁺ = 1567.8 |
| | 3-((3S,9S,12S,15S,18S,24S,27S,32aR)-18-((1H-pyrrolo[2,3-b]pyridin-3-yl)methyl)- | |
| | 12-(4-((2-(2-(2-aminoethoxy)ethoxy)acetamido)methyl)benzyl)-27-(carboxymethyl)- | |
| | 24-(4-(4-(carboxymethyl)piperazine-1-carboxamido)butyl)-8-methyl- | |
| | 1,4,7,10,13,16,19,22,25,28-decaoxo-15-(2-propoxyethyl)-9-(pyridin-3- | |
| | ylmethyl)dotriacontahydropyrrolo[1,2- | |
| | a][1,4,7,10,13,16,19,22,25,28]decaazacyclotriacontin-3-yl)propanoic acid TFA salt | |
| A10 | 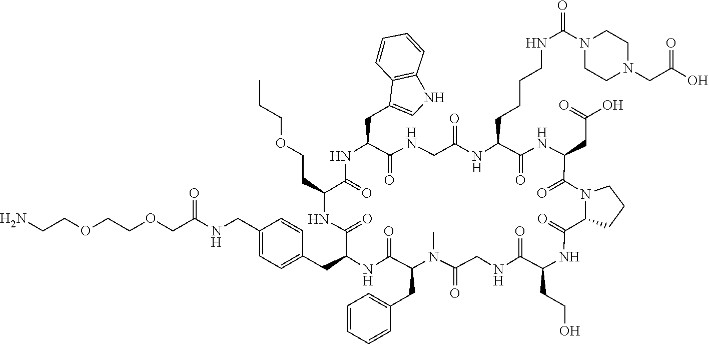 [SMILES: CCCOCC[C@@H]1NC(=O)[C@H](Cc2ccc(CNC(=O)COCCOCCN)cc2)NC(=O)[C@H](Cc2ccccc2)N(C)C(=O)CNC(=O)[C@H](CCO)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-2 t_{R} = 5.69 min [M + H]⁺ = 1537.8 |
| | 2-((3S,9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-24-(4-(4- | |
| | (carboxymethyl)piperazine-1-carboxamido)butyl)-3-(2-hydroxyethyl)-8-methyl- | |
| | 1,4,7,10,13,16,19,22,25,28-decaoxo-15-(2-propoxyethyl)dotriacontahydropyrrolo[1,2- | |
| | a][1,4,7,10,13,16,19,22,25,28]decaazacyclotriacontin-27-yl)acetic acid TFA salt | |
| A11 | 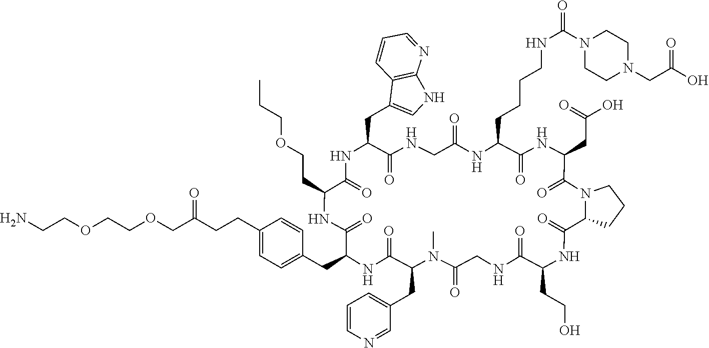 [SMILES: CCCOCC[C@@H]1NC(=O)[C@H](Cc2ccc(CCC(=O)COCCOCCN)cc2)NC(=O)[C@H](Cc2cccnc2)N(C)C(=O)CNC(=O)[C@H](CCO)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 2.43 min [M + H]⁺ = 1539.8 |
| | 2-((3S,9S,12S,15S,18S,24S,27S,32aR)-18-((1H-pyrrolo[2,3-b]pyridin-3-yl)methyl)- | |
| | 12-(4-((2-(2-(2-aminoethoxy)ethoxy)acetamido)methyl)benzyl)-24-(4-(4- | |
| | (carboxymethyl)piperazine-1-carboxamido)butyl)-3-(2-hydroxyethyl)-8-methyl- | |
| | 1,4,7,10,13,16,19,22,25,28-decaoxo-15-(2-propoxyethyl)-9-(pyridin-3- | |
| | ylmethyl)dotriacontahydropyrrolo[1,2- | |
| | a][1,4,7,10,13,16,19,22,25,28]decaazacyclotriacontin-27-yl)acetic acid | |

### 2.3 Examples Synthesized Starting from A⁷ (i.e., P1 is A⁷-Resin)

### 2.3.1 Synthesis of 2-((3S,9S,12S,15S,18S,24S,27S,32aR)-18-((1H-pyrrolo[2,3-b]pyridin-3-yl)methyl)-3-(4-aminobutyl)-9-benzyl-12,24-bis(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-27-yl)acetic acid TFA salt (Example A26)

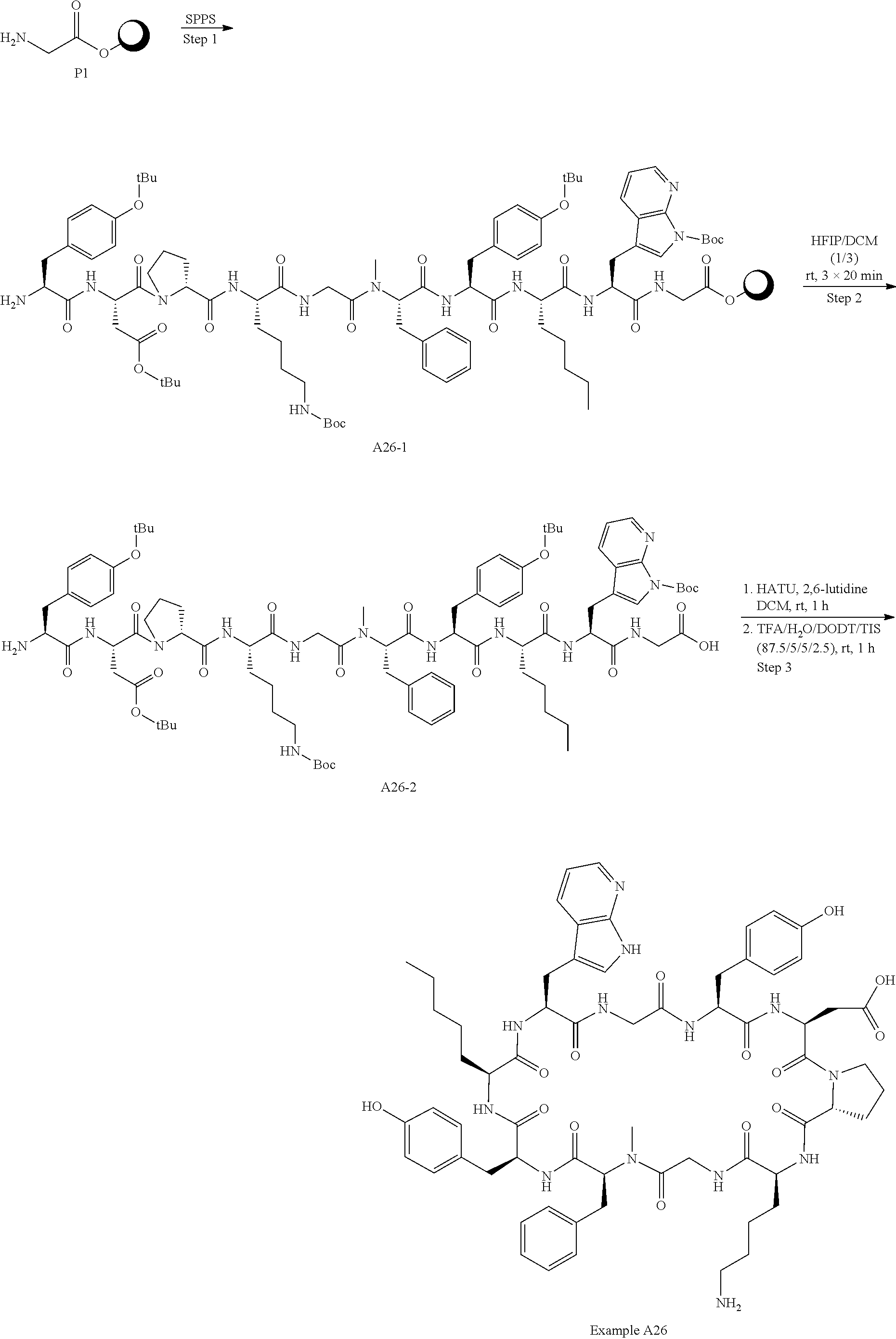

### Scheme 2.3.1. Synthesis of Example A26

### Step 1. A26-1

The assembly of the linear peptide was done on a Symphony X from Gyros Protein Technologies at rt using preloaded resin P1 (175 mg, 100 μmol, 0.572 mmol/g). The amino acids were coupled with a set of different methods.

### Method A

Fmoc-amino acid (0.2 M solution in NMP, 3 eq.), HATU (0.3 M solution in NMP, 3 eq.), DIEA (0.33 M solution in NMP, 3.3 eq.), addition by synthesizer.

### Method B

Fmoc-amino acid (0.2 M solution in NMP, 3 eq.), PyOxim (0.3 M solution in NMP, 3 eq.), DIEA (0.33 M solution in NMP, 6.6 eq.), addition by synthesizer.

### Method C (Manual Addition)

Fmoc-amino acid (3 eq.) and HATU (3 eq.) were dissolved in NMP (3 mL) and DIEA (3.3 eq.) was added. After a pre-activation time of 5 min the solution was added to the resin.

The Fmoc removal was performed using a solution of 4-methylpiperidine/DMA (1/4). Capping was performed using Ac₂O/pyridine/DMA (1/1/8). After assembly of the linear peptide A26-1 the resin was washed with DMA (5×) and DCM (5×). The coupling methods and times are summarized in Table 2.7.

TABLE 2.7

| Synthesis | Amino acid | | | |
|---|---|---|---|---|
| cycle | residue* | Amino acid | Time | Method |
| 1 | A6 | BB34 | 1 × 3 h | C |
| 2 | AS | Fmoc-Ahp-OH | 2 × 1 h | A |
| 3 | A4 | Fmoc-Tyr(tBu)-OH | 2 × 1 h | A |
| 4 | A3 | Fmoc-N-Me-Phe-OH | 2 × 1 h | A |
| 5 | A2 | Fmoc-Gly-OH | 2 × 3 h | B |
| 6 | A1 | Fmoc-Lys(Boc)-OH | 2 × 1 h | A |
| 7 | A10 | Fmoc-D-Pro-OH | 2 × 1 h | A |
| 8 | A9 | Fmoc-Asp(tBu)-OH | 2 × 2 h | B |
| 9 | A8 | Fmoc-Tyr(tBu)-OH | 2 × 1 h | A |

*Refers to the corresponding amino acid residue of cyclic peptide, P, as found, e.g., in Formulae (I), (Ia), (Ib), (Ic), (Id), or in the Table of Example III.

### Step 2. A26-2

To the resin A26-1 (calculated with 100 μmol) was added HFIP/DCM (1/3) (3 mL). The resin was shaken at rt for 20 min. The cleavage solution was filtered off and the procedure was repeated two times. The resin was washed with HFIP/DCM (1/3) (1 mL). The combined cleavage and washing solutions were concentrated to dryness in vacuo. The residue was treated with toluene and the resulting mixture was concentrated to dryness in vacuo. The residue was dried in high vacuum at 40° C. and A26-2 was obtained as an off-white solid. LCMS method P-1, t_{R}=5.98 min, [M+H]⁺=1642.9.

### Step 3. Example A26

Step 3-1: To a clear solution of the linear peptide A26-2 (calculated with 100 μmol) in DCM (50 mL) at rt were added HATU (76.0 mg, 2 eq., 200 mol) and 2,6-lutidine (268 mg, 290 μL, 25 eq., 2.5 mmol). The reaction was stirred at rt for 1 h, then concentrated to dryness in vacuo.

Step 3-2: The residue was dissolved in TFA/H₂O/DODT/TIS (87.5/5/5/2.5) (7 mL) and the reaction was stirred at rt for 1 h. The clear solution was then poured onto cold methyl tert-butyl ether/heptanes (1/1) (40 mL), giving a precipitate. The suspension was centrifuged and the solvent was decanted. The residue was washed with cold methyl tert-butyl ether/heptanes (1/1) (2×15 mL), then dried in high vacuum at 40° C. for 1 h. The product was isolated by preparative HPLC (Column: XSelect Peptide CSH C18 OBD Prep Column, 130 Å, 5 μm, 30 mm×250 mm; Eluent A: H₂O+0.1% TFA and eluent B: ACN). Pure fractions were combined and lyophilized to afford Example A26 (56.3 mg, 38 μmol, 38% yield, TFA salt) as a white solid. LCMS method P-1, t_{R}=3.01 min, [M+H]⁺=1256.6.

### 2.3.2 Synthesis of 2-((9S,12S,15S,18S,24S,27S,32aR)-18-((1H-indol-3-yl)methyl)-9-benzyl-12,24-bis(4-hydroxybenzyl)-2,8-dimethyl-1,4,7,10,13,16,19,22,25,28-decaoxo-15-pentyldotriacontahydropyrrolo[1,2-a][1,4,7,10,13,16,19,22,25,28]decaazacyclotriacontin-27-yl)acetic acid (Example A73)

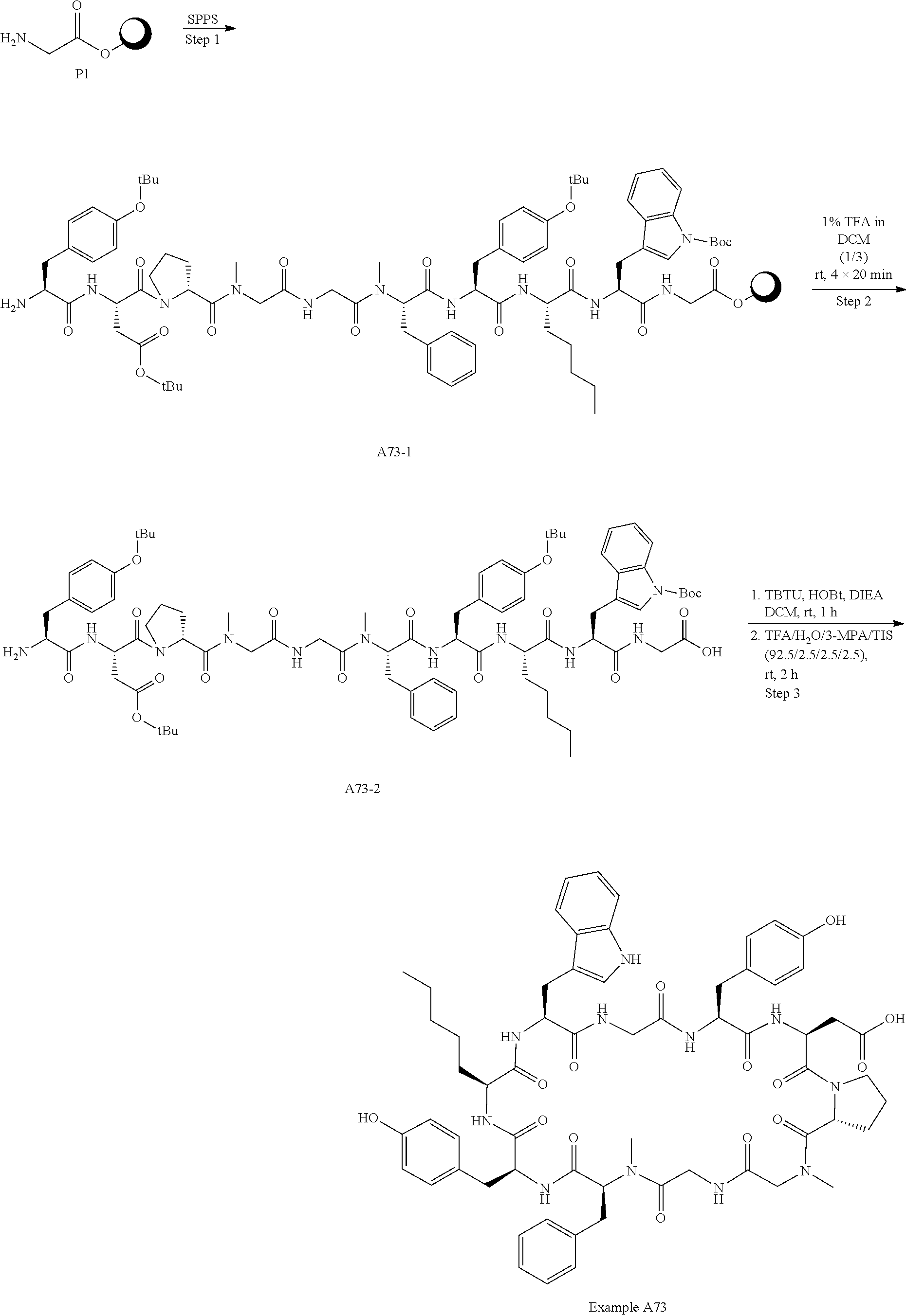

### Scheme 2.3.2. Synthesis of Example A73

### Step 1. A73-1

The assembly of the linear peptide was done manually at rt using preloaded resin P1 (0.200 mmol). The amino acids were coupled with a set of different methods.

### Method A

Fmoc-amino acid (3.00 eq.), HATU (2.85 eq.) and DIEA (6.0 eq.) in DMF (3 mL). Agitation with N₂.

### Method B

Fmoc-amino acid (6.00 eq.), HATU (5.70 eq.) and DIEA (12.0 eq.) in DMF (3 mL). Agitation with N₂.

The Fmoc removal was performed using piperidine/DMF (1/4) (10.0 mL). The resin was agitated with N₂ at rt for 20 min, washed with DMF (10.0 mL×5) and filtered.

The coupling methods are summarized in Table 2.8.

TABLE 2.8

| Synthesis | Amino acid | | |
|---|---|---|---|
| cycle | residue* | Amino acid | Method |
| 1 | A⁶ | Fmoc-Trp(Boc)-OH | A |
| 2 | A⁵ | Fmoc-Ahp-OH | A |
| 3 | A⁴ | Fmoc-Tyr(tBu)-OH | A |
| 4 | A³ | Fmoc-N-Me-Phe-OH | A |
| 5 | A² | Fmoc-Gly-OH | A |
| 6 | A¹ | Fmoc-Sar-OH | A |
| 7 | A¹⁰ | Fmoc-D-Pro-OH | B |
| 8 | A⁹ | Fmoc-Asp(OtBu)-OH | B |
| 9 | A⁸ | Fmoc-Tyr(tBu)-OH | B |

*Refers to the corresponding amino acid residue of cyclic peptide, P, as found, e.g., in Formulae (I), (Ia), (Ib), (Ic), (Id), or in the Table of Example III.

Coupling time ranged from 40 min to 90 min. The coupling reaction was monitored by ninhydrin/chloranil test. After the last cycle, the resin was washed with MeOH (3×).

### Step 2. A73-2

To the resin A73-1 was added 1% TFA in DCM (20 mL) and the suspension was agitated at rt with N₂ for 20 min. The above step was repeated (3×). The combined cleavage solutions were collected in a 500 mL round bottom flask.

### Step 3. Example A73

Step 3-1: The mixture from Step 2 (A73-2, calculated with 200 μmol) was diluted with DCM to 200 mL. Then TBTU (128 mg, 0.400 mmol, 2.00 eq.) and HOBt (54.0 mg, 0.400 mmol, 2.00 eq.) were added. DIEA was added to the mixture to adjust the pH to 8. The mixture was stirred at rt for 1 h. The mixture was washed with 1 M aq. HCl (3×). The organic layer was concentrated to dryness in vacuo.

### Step 3-2: TFA/H₂O/3-MPA/TIS (92.5/2.5/2.5/2.5) (5 mL) was Added to the Residue from Step

3-1 and the Reaction Mixture was Stirred for 2 h. The Crude Peptide was Precipitated with Cold Isopropyl ether (45 mL). The suspension was centrifuged and the solvent was decanted. The residue was washed with isopropyl ether (2×45 mL), then dried under high vacuum for 2 h. The product was isolated by preparative HPLC (Columns: Gemini C18, 110 A, 5 μm, 30 mm×150 mm+Luna C18, 100 A, 10 m, 25 mm×120 mm; Eluent A: H₂O+0.075% TFA and eluent B: ACN). Pure fractions were combined and lyophilized to afford Example A73 (93.1 mg, 77.7 μmol, 38% yield) as a white solid. LC-MS Method P-5, t_{R}=1.55 min, [M+H]⁺=1198.7.

### 2.3.3 Synthesis of 2-((6S,9S,15S,18S,21S,24S,29aS,34aR)-15-((1H-indol-3-yl)methyl)-24-benzyl-9,21-bis(4-hydroxybenzyl)-25-methyl-5,8,11,14,17,20,23,26,29,34-decaoxo-18-pentyldotriacontahydro-1H,5H-dipyrrolo[1,2-a:1′,2′-b₁][1,4,7,10,13,16,19,22,25,28]decaazacyclotriacontin-6-yl)acetic acid (Example A51)

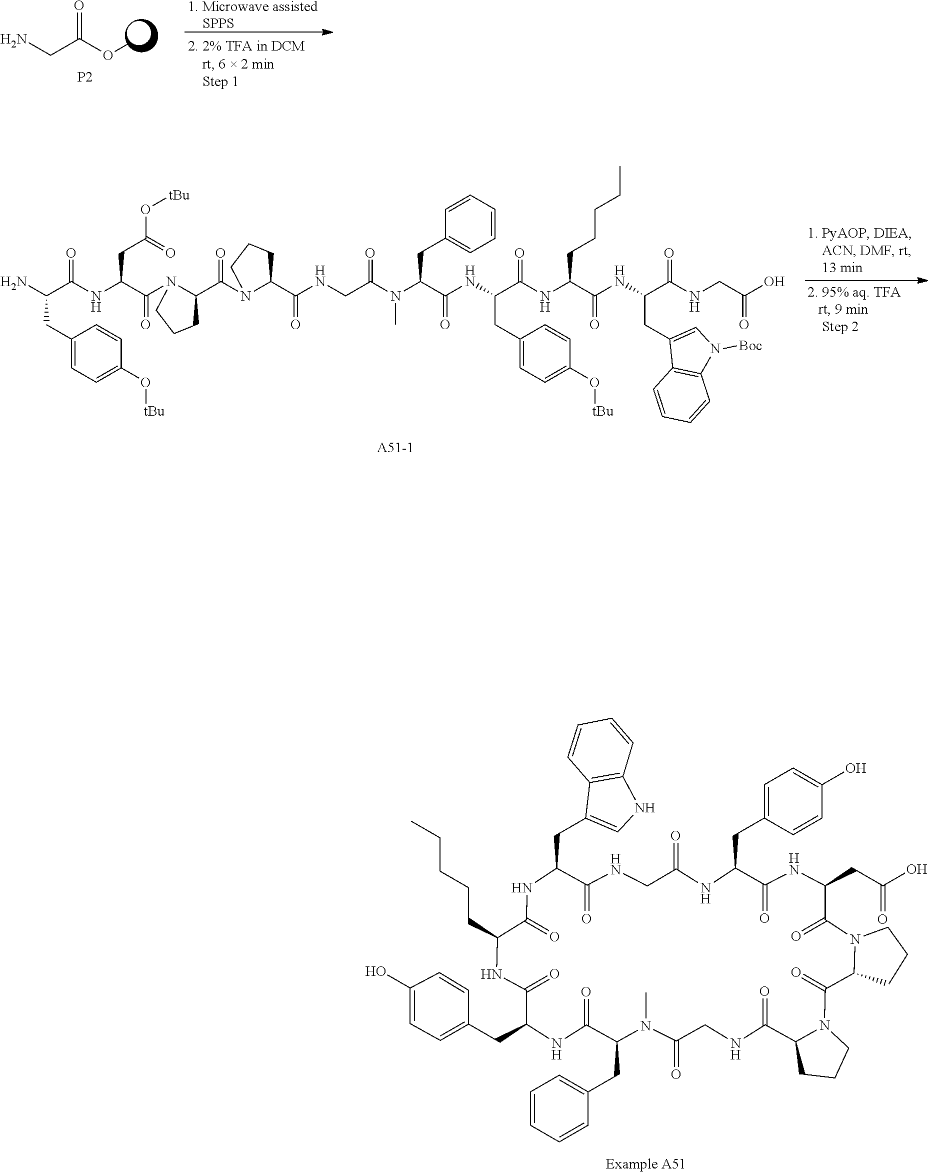

### Scheme 2.3.3. Synthesis of Example A51

### Step 1. A51-1

Step 1-1. The assembly of the linear peptide was done on a CEM Liberty Prime 1.0 from CEM using preloaded H-Gly-Trt-Amide-MBHA resin P2 (209.8 mg, 102 μmol, 0.486 mmol/g). The amino acids were coupled with a set of different methods.

### Method A

Fmoc-amino acid (0.2 M solution in 0.19 M OxymaPure in NMP, 3 eq.), DIC (0.64 M solution in NMP, 4.5 eq.), NMP (1.8 mL), addition by synthesizer, 4 min at 75° C. The Fmoc removal was performed using 10% 4-methylpiperidine in 0.3 M OxymaPure in NMP (4 mL) at 70° C. for 1×1 min/1×1.5 min.

### Method B

Fmoc-amino acid (0.2 M solution in 0.19 M OxymaPure in NMP, 3 eq.), DIC (0.64 M solution in NMP, 4.5 eq.), NMP (1.8 mL) addition by synthesizer, 8 min at 75° C. The Fmoc removal was performed using 10% 4-methylpiperidine in 0.3 M OxymaPure in NMP (4 mL) at 70° C. for 1×1 min/1×1.5 min.

### Method C

Fmoc-amino acid (0.2 M solution in 0.19 M OxymaPure in NMP, 3 eq.), DIC (0.64 M solution in NMP, 4.5 eq.), NMP (1.8 mL), addition by synthesizer, 8 min at 75° C. The Fmoc removal was performed using 10% 4-methylpiperidine in 0.3 M OxymaPure in NMP (4 mL) for 1×20 s at 50° C./1×1 min at 55° C.

Capping was performed using Ac₂O/pyridine/DMA (1/:1/8) at 60° C. for 45 s. Final Fmoc deprotection was performed using 10% 4-methylpiperidine in 0.3 M OxymaPure in NMP at 70° C., 2×1 min. After the assembly of the linear peptide the resin was washed with DMA (5×) and DCM (5×).

The coupling methods and summarized in Table 2.9.

TABLE 2.9

| Synthesis | Amino acid | | Time/ | |
|---|---|---|---|---|
| cycle | residue* | Amino acid | temperature | Method |
| 1 | A6 | Fmoc-Trp(Boc)-OH | 1 × 4 min, 75° C. | A |
| 2 | A5 | Fmoc-Ahp-OH | 1 × 4 min, 75° C. | A |
| 3 | A4 | Fmoc-Tyr(tBu)-OH | 1 × 4 min, 75° C. | A |
| 4 | A3 | Fmoc-N-Me-Phe-OH | 1 × 8 min, 75° C. | B |
| 5 | A2 | Fmoc-Gly-OH | 1 × 8 min, 75° C. | B |
| 6 | A1 | Fmoc-Pro-OH | 1 × 8 min, 75° C. | C |
| 7 | A10 | Fmoc-D-Pro-OH | 1 × 4 min, 75° C. | A |
| 8 | A9 | Fmoc-Asp(tBu)-OH | 1 × 4 min, 75° C. | A |
| 9 | A8 | Fmoc-Tyr(tBu)-OH | 1 × 4 min 75° C. | A |

*Refers to the corresponding amino acid residue of cyclic peptide, P, as found, e.g., in Formulae (I), (Ia), (Ib), (Ic), (Id), or in the Table of Example III.

Step 1-2: To the resin was added 2% TFA in DCM (4 mL). The resin was shaken at rt for 2 min. The cleavage solution was filtered off and the procedure was repeated 5 times. The combined cleavage solutions were concentrated to dryness in vacuo. The residue was lyophilized from dioxane to afford the crude peptide A51-1 (71.6 mg, 44.1 μmol, TFA salt) as a solid. LCMS method P-1, t_{R}=6.09 min, [M+H]⁺=1510.6.

### Step 2. Example A51

Step 2-1: A51-1 (71.6 mg, 44.1 μmol, 1 eq, TFA salt) was dissolved ACN (2.4 mL) and DIEA (23.0 mg, 31.0 μl, 4 eq., 176 μmol) was added. The resulting solution was continuously added (over 5 min) to a freshly prepared solution of PyAOP (93.8 mg, 4 eq., 176 μmol) and DIEA (46.0 mg, 62.0 μL, 8 eq., 353 μmol) in ACN (4.7 mL) and DMF (1.5 mL). The reaction mixture was stirred at rt for 13 min, then concentrated to dryness in vacuo to afford the cyclic protected peptide as a yellow oil.

Step 2-2: The residue was dissolved in TFA/H₂O (95/5) (2 mL) and the reaction mixture was stirred at rt for 9 min. The clear solution was then poured onto cold diisopropylether/PE (1/1) (25 mL), giving a precipitate. The suspension was centrifuged and the solvent was decanted. The residue was washed with cold diisopropylether/PE (1/1) (1×25 mL), then dried in high vacuum at rt. The residue was dissolved in 0.02 M aq. NaHCO₃/ACN (1/1) (1.5 mL), warmed up to 50° C. for 20 min and 20 min at rt the resulted emulsion was dissolved with 95% aq. TFA (400 L). The product was isolated by preparative reversed-phase HPLC (SunFire C18 OBD Prep Column, 100 Å, 5 μm, 50 mm×150 mm; Eluent A: H₂O+0.1% TFA and eluent B: ACN). Pure fractions were combined and lyophilized to afford Example A51 (19.04 mg, 15.0 μmol, 33.9% yield) as a white solid. LCMS method P-1, t_{R}=4.79 min, [M+H]⁺=1224.4.

Examples A12 to A25, A27, A31 to A38, A40 to A49 of Table 2.10 were synthesized in analogy to Example A26 (Scheme 2.3.1).

Examples A59 to A72 of Table 2.10 were synthesized in analogy to Example A73 (Scheme 2.3.2).

Examples A52 to A54, A56 to A58 of Table 2.10 were synthesized in analogy to Example A51 (Scheme 2.3.3).

TABLE 2.10
Examples synthesized starting from position 7

| Ex- | | |
|---|---|---|
| ample | | |
| No. | Structure/Example Name | LCMS |
| A12 | 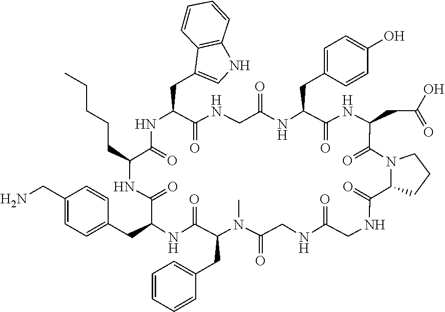 [SMILES: CCCCC[C@@H]1NC(=O)[C@H](Cc2ccc(CN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.79 min [M + H]⁺ = 1197.6 |
| | 2-((9S,12S,15S,18S,24S,27S,32aR)-18-((1H-indol-3-yl)methyl)-12-(4- | |
| | (aminomethyl)benzyl)-9-benzyl-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A13 | 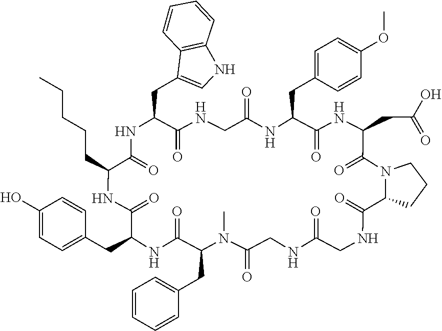 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(OC)cc2)NC(=O)CNC(=O)[C@H](Cc2c[nH]c3ccccc23)NC1=O] | Method P-1 t_{R} = 5.09 min [M + H]⁺ = 1198.6 |
| | 2-((9S,12S,15S,18S,24S,27S,32aR)-18-((1H-indol-3-yl)methyl)-9-benzyl-12-(4- | |
| | hydroxybenzyl)-24-(4-methoxybenzyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid | |
| A14 | 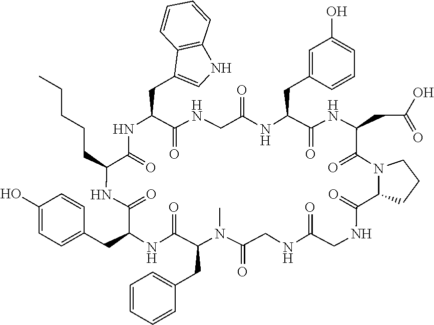 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2cccc(O)c2)NC(=O)CNC(=O)[C@H](Cc2c[nH]c3ccccc23)NC1=O] | Method P-1 t_{R} = 4.81 min [M + H]⁺ = 1184.6 |
| | 2-((9S,12S,15S,18S,24S,27S,32aR)-18-((1H-indol-3-yl)methyl)-9-benzyl-24-(3- | |
| | hydroxybenzyl)-12-(4-hydroxybenzyl)-8-methyl-1,4,7,10,13,16,19,22,25,28-decaoxo- | |
| | 15-pentyldotriacontahydropyrrolo[1,2- | |
| | a][1,4,7,10,13,16,19,22,25,28]decaazacyclotriacontin-27-yl)acetic acid | |
| A15 | 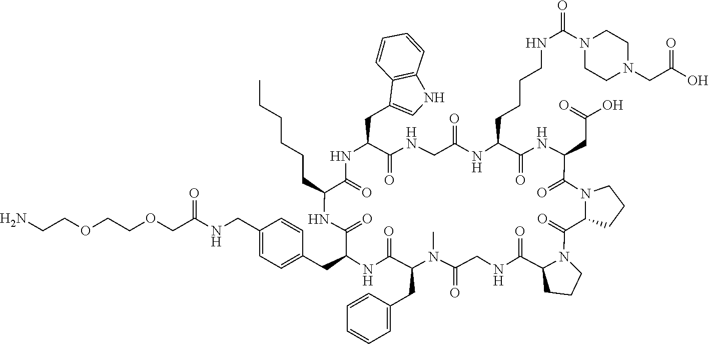 [SMILES: CCCCCC[C@@H]1NC(=O)[C@H](Cc2ccc(CNC(=O)COCCOCCN)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.72 min [M + H]⁺ = 1531.8 |
| | 2-(4-((4-((6S,9S,15S,18S,21S,24S,29aS,34aR)-15-((1H-indol-3-yl)methyl)-21-(4-((2- | |
| | (2-(2-aminoethoxy)ethoxy)acetamido)methyl)benzyl)-24-benzyl-6-(carboxymethyl)- | |
| | 18-hexyl-25-methyl-5,8,11,14,17,20,23,26,29,34-decaoxodotriacontahydro-1H,5H- | |
| | dipyrrolo[1,2-a:1′,2′-b₁][1,4,7,10,13,16,19,22,25,28]decaazacyclotriacontin-9- | |
| | yl)butyl)carbamoyl)piperazin-1-yl)acetic acid TFA salt | |
| A16 | 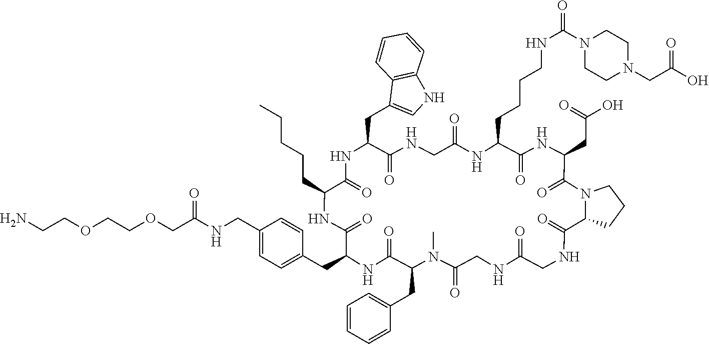 [SMILES: CCCCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.41 min [M + H]⁺ = 1477.8 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-24-(4-(4- | |
| | (carboxymethyl)piperazine-1-carboxamido)butyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A17 | 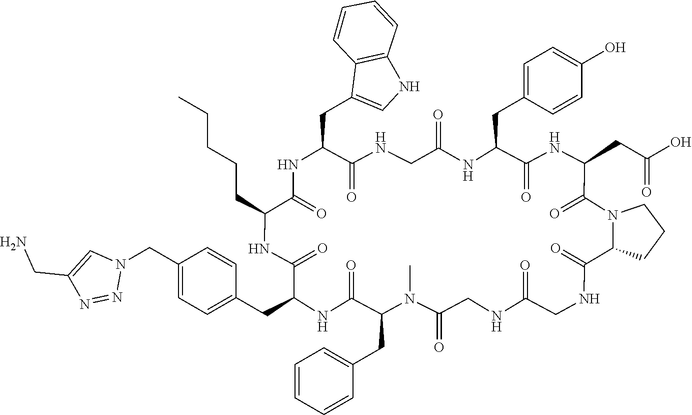 [SMILES: CCCCC[C@@H]1NC(=O)[C@H](Cc2ccc(Cn3cc(CN)nn3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.92 min [M + H]⁺ = 1278.6 |
| | 2-((9S,12S,15S,18S,24S,27S,32aR)-18-((1H-indol-3-yl)methyl)-12-(4-((4- | |
| | (aminomethyl)-1H-1,2,3-triazol-1-yl)methyl)benzyl)-9-benzyl-24-(4-hydroxybenzyl)- | |
| | 8-methyl-1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid | |
| A18 | 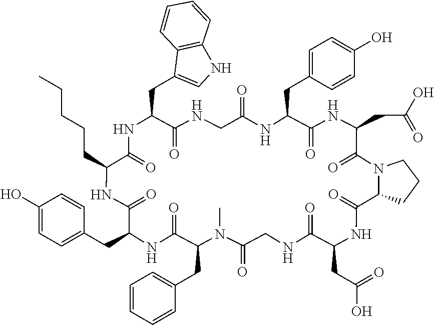 [SMILES: CCCCC[C@@H]1NC(=O)[C@H](Cc2ccc(O)cc2)NC(=O)[C@H](Cc2ccccc2)N(C)C(=O)CNC(=O)[C@H](CC(=O)O)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.68 min [M + H]⁺ = 1242.6 |
| | 2,2′-((3S,9S,12S,15S,18S,24S,27S,32aR)-18-((1H-indol-3-yl)methyl)-9-benzyl-12,24- | |
| | bis(4-hydroxybenzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontine-3,27-diyl)diacetic acid | |
| A19 | 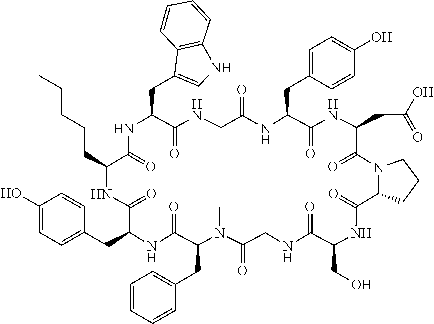 [SMILES: CCCCC[C@@H]1NC(=O)[C@H](Cc2ccc(O)cc2)NC(=O)[C@H](Cc2ccccc2)N(C)C(=O)CNC(=O)[C@H](CO)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.67 min [M + H]⁺ = 1214.6 |
| | 2-((3S,9S,12S,15S,18S,24S,27S,32aR)-18-((1H-indol-3-yl)methyl)-9-benzyl-12,24- | |
| | bis(4-hydroxybenzyl)-3-(hydroxymethyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid | |
| A20 | 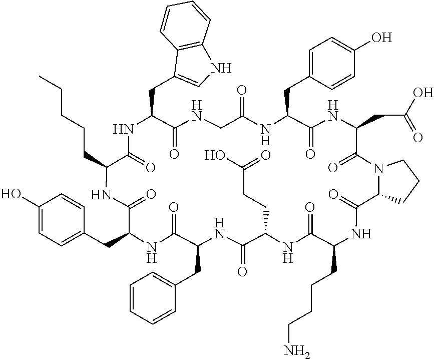 [SMILES: CCCCC[C@@H]1NC(=O)[C@H](Cc2ccc(O)cc2)NC(=O)[C@H](Cc2ccccc2)NC(=O)[C@H](CCC(=O)O)NC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.89 min [M + H]⁺ = 1313.6 |
| | 3-((3S,6S,9S,12S,15S,18S,24S,27S,32aR)-18-((1H-indol-3-yl)methyl)-3-(4- | |
| | aminobutyl)-9-benzyl-27-(carboxymethyl)-12,24-bis(4-hydroxybenzyl)- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-6-yl)propanoic acid | |
| A21 | 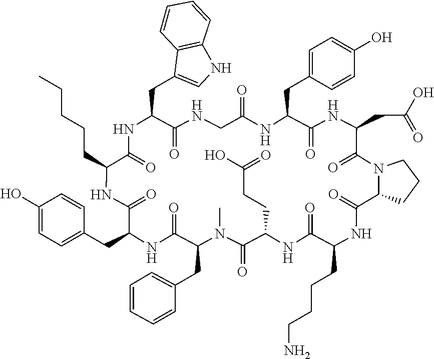 [SMILES: CCCCC[C@@H]1NC(=O)[C@H](Cc2ccc(O)cc2)NC(=O)[C@H](Cc2ccccc2)N(C)C(=O)[C@H](CCC(=O)O)NC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.95 min [M + H]⁺ = 1327.6 |
| | 3-((3S,6S,9S,12S,15S,18S,24S,27S,32aR)-18-((1H-indol-3-yl)methyl)-3-(4- | |
| | aminobutyl)-9-benzyl-27-(carboxymethyl)-12,24-bis(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-6-yl)propanoic acid TFA salt | |
| A22 | 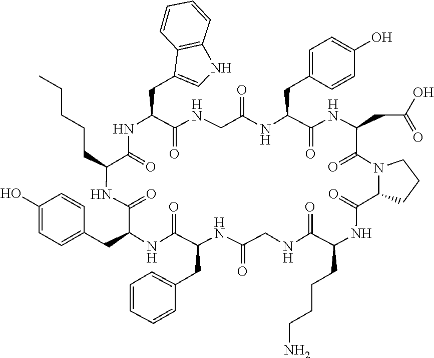 [SMILES: CCCCC[C@@H]1NC(=O)[C@H](Cc2ccc(O)cc2)NC(=O)[C@H](Cc2ccccc2)NC(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.90 min [M + H]⁺ = 1241.6 |
| | 2-((3S,9S,12S,15S,18S,24S,27S,32aR)-18-((1H-indol-3-yl)methyl)-3-(4-aminobutyl)- | |
| | 9-benzyl-12,24-bis(4-hydroxybenzyl)-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid TFA salt | |
| A23 | 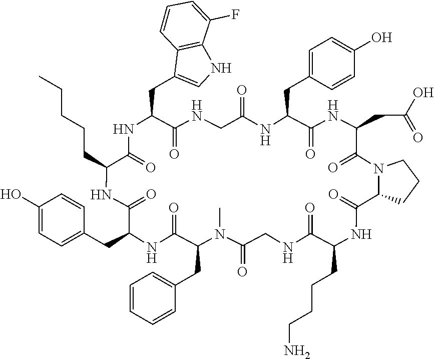 [SMILES: CCCCC[C@@H]1NC(=O)[C@H](Cc2ccc(O)cc2)NC(=O)[C@H](Cc2ccccc2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3c(F)cccc23)NC1=O] | Method P-1 t_{R} = 4.11 min [M + H]⁺ = 1273.6 |
| | 2-((3S,9S,12S,15S,18S,24S,27S,32aR)-3-(4-aminobutyl)-9-benzyl-18-((7-fluoro-1H- | |
| | indol-3-yl)methyl)-12,24-bis(4-hydroxybenzyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid TFA salt | |
| A24 | 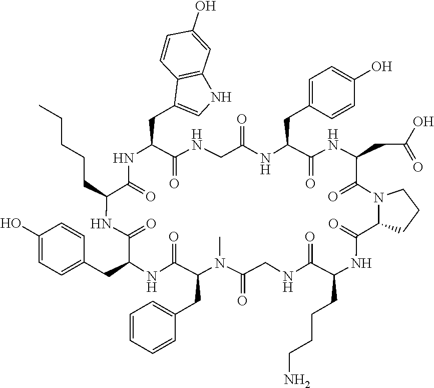 [SMILES: CCCCC[C@@H]1NC(=O)[C@H](Cc2ccc(O)cc2)NC(=O)[C@H](Cc2ccccc2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3cc(O)ccc23)NC1=O] | Method P-1 t_{R} = 3.52 min [M + H]⁺ = 1271.6 |
| | 2-((3S,9S,12S,15S,18S,24S,27S,32aR)-3-(4-aminobutyl)-9-benzyl-18-((6-hydroxy- | |
| | 1H-indol-3-yl)methyl)-12,24-bis(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A25 | 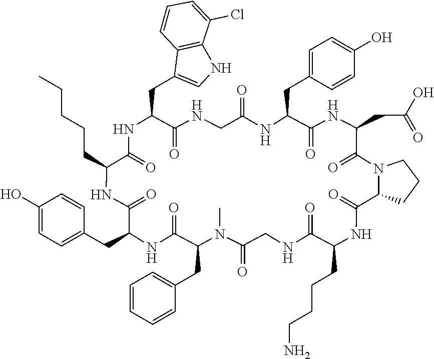 [SMILES: CCCCC[C@@H]1NC(=O)[C@H](Cc2ccc(O)cc2)NC(=O)[C@H](Cc2ccccc2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3c(Cl)cccc23)NC1=O] | Method P-1 t_{R} = 4.30 min [M + H]⁺ = 1289.6 |
| | 2-((3S,9S,12S,15S,18S,24S,27S,32aR)-3-(4-aminobutyl)-9-benzyl-18-((7-chloro-1H- | |
| | indol-3-yl)methyl)-12,24-bis(4-hydroxybenzyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid TFA salt | |
| A27 | 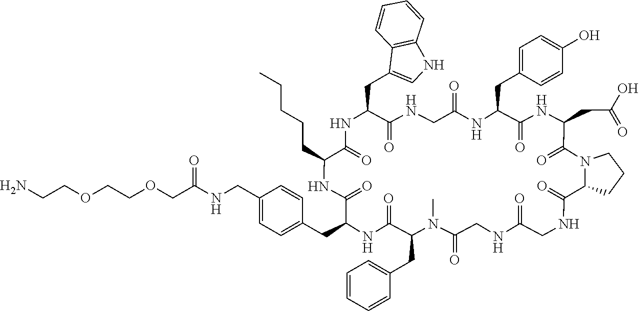 [SMILES: CCCCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.84 min [M + H]⁺ = 1342.6 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-24-(4-hydroxybenzyl)-8- | |
| | methyl-1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid | |
| A31 | 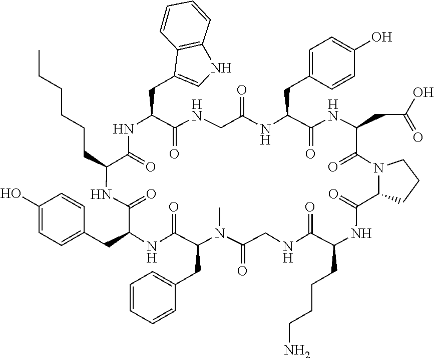 [SMILES: CCCCCC[C@@H]1NC(=O)[C@H](Cc2ccc(O)cc2)NC(=O)[C@H](Cc2ccccc2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.26 min [M + H]⁺ = 1269.6 |
| | 2-((3S,9S,12S,15S,18S,24S,27S,32aR)-18-((1H-indol-3-yl)methyl)-3-(4-aminobutyl)- | |
| | 9-benzyl-15-hexyl-12,24-bis(4-hydroxybenzyl)-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-27-yl)acetic acid TFA salt | |
| A32 | 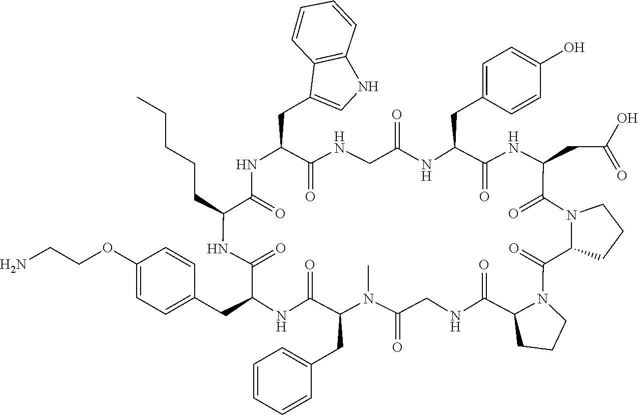 [SMILES: CCCCC[C@@H]1NC(=O)[C@H](Cc2ccc(OCCN)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.01 min [M + H]⁺ = 1267.6 |
| | 2-((6S,9S,15S,18S,21S,24S,29aS,34aR)-15-((1H-indol-3-yl)methyl)-21-(4-(2- | |
| | aminoethoxy)benzyl)-24-benzyl-9-(4-hydroxybenzyl)-25-methyl- | |
| | 5,8,11,14,17,20,23,26,29,34-decaoxo-18-pentyldotriacontahydro-1H,5H- | |
| | dipyrrolo[1,2-a:1′,2′-b₁][1,4,7,10,13,16,19,22,25,28]decaazacyclotriacontin-6- | |
| | yl)acetic acid | |
| A33 | 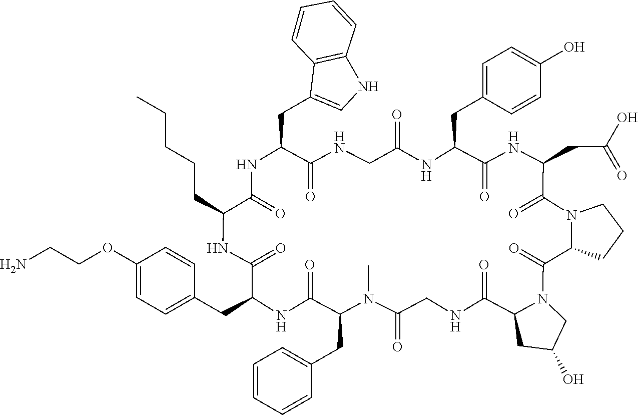 [SMILES: CCCCC[C@@H]1NC(=O)[C@H](Cc2ccc(OCCN)cc2)NC(=O)[C@H](Cc2ccccc2)N(C)C(=O)CNC(=O)[C@@H]2C[C@@H](O)CN2C(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.86 min [M + H]⁺ = 1283.6 |
| | 2-((6S,9S,15S,18S,21S,24S,29aS,31R,34aR)-15-((1H-indol-3-yl)methyl)-21-(4-(2- | |
| | aminoethoxy)benzyl)-24-benzyl-31-hydroxy-9-(4-hydroxybenzyl)-25-methyl- | |
| | 5,8,11,14,17,20,23,26,29,34-decaoxo-18-pentyldotriacontahydro-1H,5H- | |
| | dipyrrolo[1,2-a:1′,2′-b₁][1,4,7,10,13,16,19,22,25,28]decaazacyclotriacontin-6- | |
| | yl)acetic acid TFA salt | |
| A34 | 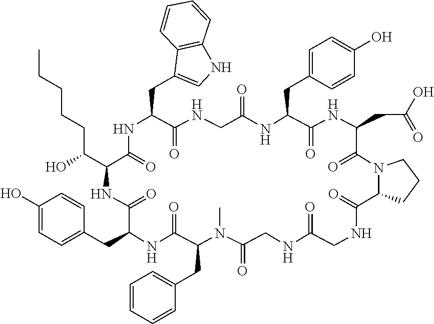 [SMILES: CCCCC[C@@H](O)[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.60 min [M + H]⁺ = 1214.6 |
| | 2-((9S,12S,15S,18S,24S,27S,32aR)-18-((1H-indol-3-yl)methyl)-9-benzyl-12,24-bis(4- | |
| | hydroxybenzyl)-15-((R)-1-hydroxyhexyl)-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-27-yl)acetic acid | |
| A35 | 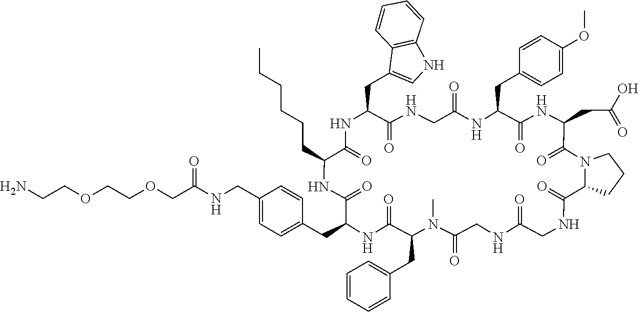 [SMILES: CCCCCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(OC)cc2)NC(=O)CNC(=O)[C@H](Cc2c[nH]c3ccccc23)NC1=O] | Method P-1 t_{R} = 4.46 min [M + H]⁺ = 1370.7 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-15-hexyl-24-(4- | |
| | methoxybenzyl)-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-27-yl)acetic acid TFA salt | |
| A36 | 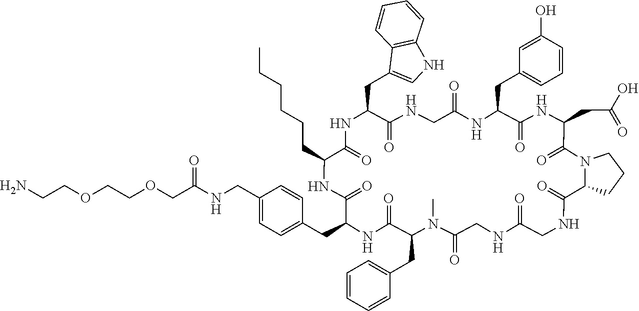 [SMILES: CCCCCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2cccc(O)c2)NC(=O)CNC(=O)[C@H](Cc2c[nH]c3ccccc23)NC1=O] | Method P-1 t_{R} = 4.24 min [M + H]⁺ = 1356.7 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-15-hexyl-24-(3- | |
| | hydroxybenzyl)-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-27-yl)acetic acid TFA salt | |
| A37 | 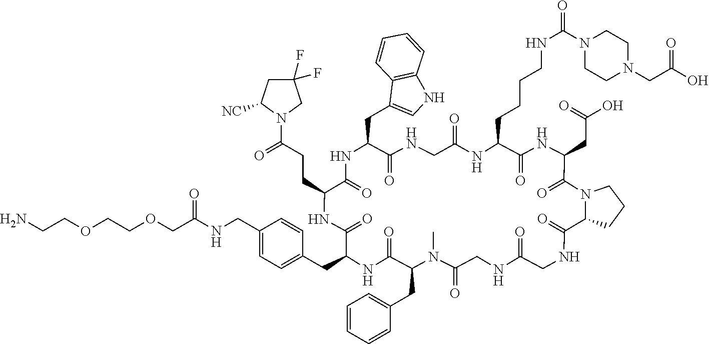 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCN)cc2)NC(=O)[C@@H]1Cc1ccccc1] | Method P-1 t_{R} = 2.85 min [M + H]⁺ = 1593.7 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-9-benzyl-24-(4-(4- | |
| | (carboxymethyl)piperazine-1-carboxamido)butyl)-15-(3-((S)-2-cyano-4,4- | |
| | difluoropyrrolidin-1-yl)-3-oxopropyl)-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-27-yl)acetic acid TFA salt | |
| A38 | 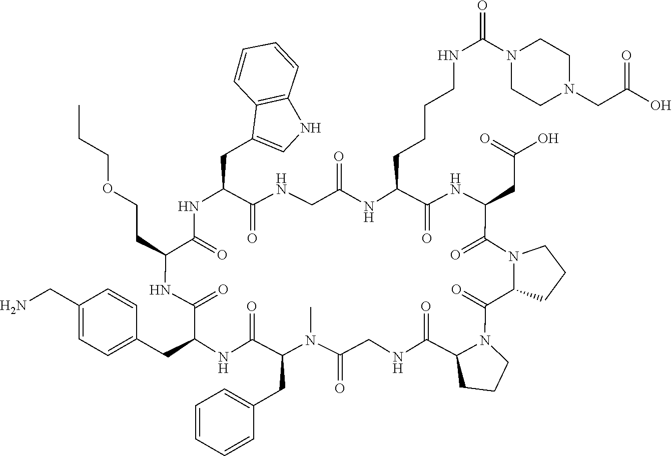 [SMILES: CCCOCC[C@@H]1NC(=O)[C@H](Cc2ccc(CN)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.11 min [M + H]⁺ = 1388.7 |
| | 2-(4-((4-((6S,9S,15S,18S,21S,24S,29aS,34aR)-15-((1H-indol-3-yl)methyl)-21-(4- | |
| | (aminomethyl)benzyl)-24-benzyl-6-(carboxymethyl)-25-methyl- | |
| | 5,8,11,14,17,20,23,26,29,34-decaoxo-18-(2-propoxyethyl)dotriacontahydro-1H,5H- | |
| | dipyrrolo[1,2-a:1′,2′-b₁][1,4,7,10,13,16,19,22,25,28]decaazacyclotriacontin-9- | |
| | yl)butyl)carbamoyl)piperazin-1-yl)acetic acid TFA salt | |
| A40 | 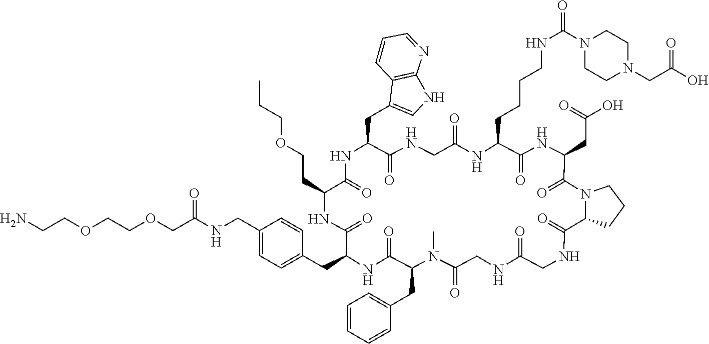 [SMILES: CCCOCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 4.06 min [M + H]⁺ = 1494.7 |
| | 2-((9S,12S,15S,18S,24S,27S,32aR)-18-((1H-pyrrolo[2,3-b]pyridin-3-yl)methyl)-12- | |
| | (4-((2-(2-(2-aminoethoxy)ethoxy)acetamido)methyl)benzyl)-9-benzyl-24-(4-(4- | |
| | (carboxymethyl)piperazine-1-carboxamido)butyl)-8-methyl- | |
| | 1,4,7,10,13,16,19,22,25,28-decaoxo-15-(2- | |
| | propoxyethyl)dotriacontahydropyrrolo[1,2- | |
| | a][1,4,7,10,13,16,19,22,25,28]decaazacyclotriacontin-27-yl)acetic acid TFA salt | |
| A41 | 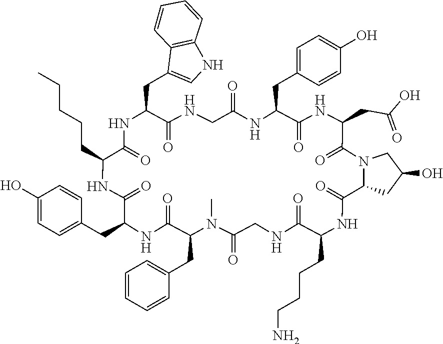 [SMILES: CCCCC[C@@H]1NC(=O)[C@H](Cc2ccc(O)cc2)NC(=O)[C@H](Cc2ccccc2)N(C)C(=O)CNC(=O)[C@H](CCCCN)NC(=O)[C@H]2C[C@H](O)CN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.95 min [M + H]⁺ = 1271.6 |
| | 2-((3S,9S,12S,15S,18S,24S,27S,31S,32aR)-18-((1H-indol-3-yl)methyl)-3-(4- | |
| | aminobutyl)-9-benzyl-31-hydroxy-12,24-bis(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A42 | 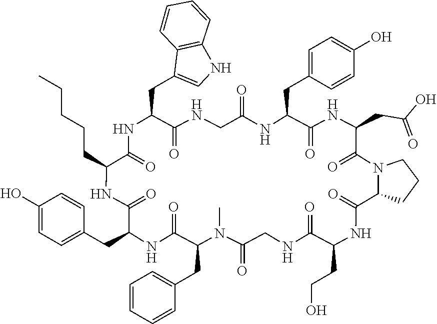 [SMILES: CCCCC[C@@H]1NC(=O)[C@H](Cc2ccc(O)cc2)NC(=O)[C@H](Cc2ccccc2)N(C)C(=O)CNC(=O)[C@H](CCO)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.62 min [M + H]⁺ = 1228.6 |
| | 2-((3S,9S,12S,15S,18S,24S,27S,32aR)-18-((1H-indol-3-yl)methyl)-9-benzyl-12,24- | |
| | bis(4-hydroxybenzyl)-3-(2-hydroxyethyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid | |
| A43 | 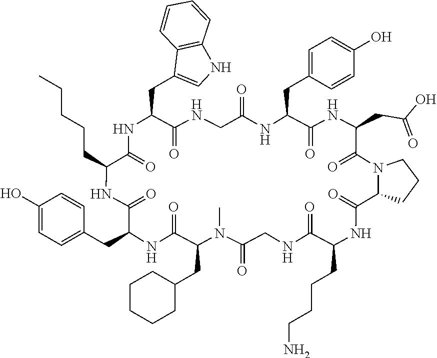 [SMILES: CCCCC[C@@H]1NC(=O)[C@H](Cc2ccc(O)cc2)NC(=O)[C@H](CC2CCCCC2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.36 min [M + H]⁺ = 1261.7 |
| | 2-((3S,9S,12S,15S,18S,24S,27S,32aR)-18-((1H-indol-3-yl)methyl)-3-(4-aminobutyl)- | |
| | 9-(cyclohexylmethyl)-12,24-bis(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A44 | 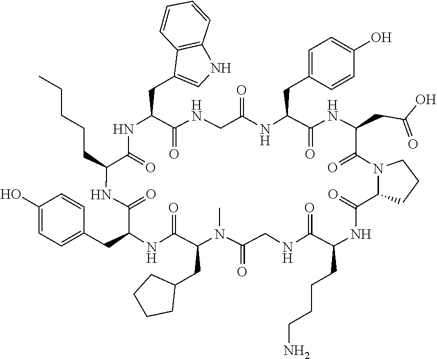 [SMILES: CCCCC[C@@H]1NC(=O)[C@H](Cc2ccc(O)cc2)NC(=O)[C@H](CC2CCCC2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.20 min [M + H]⁺ = 1247.7 |
| | 2-((3S,9S,12S,15S,18S,24S,27S,32aR)-18-((1H-indol-3-yl)methyl)-3-(4-aminobutyl)- | |
| | 9-(cyclopentylmethyl)-12,24-bis(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A45 | 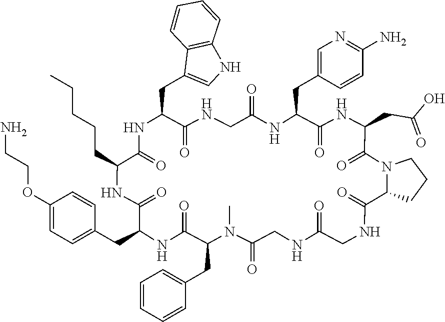 [SMILES: CCCCC[C@@H]1NC(=O)[C@H](Cc2ccc(OCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(N)nc2)NC(=O)CNC(=O)[C@H](Cc2c[nH]c3ccccc23)NC1=O] | Method P-2 t_{R} = 6.01 min [M + H]⁺ = 1227.6 |
| | 2-((9S,12S,15S,18S,24S,27S,32aR)-18-((1H-indol-3-yl)methyl)-12-(4-(2- | |
| | aminoethoxy)benzyl)-24-((6-aminopyridin-3-yl)methyl)-9-benzyl-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A46 | 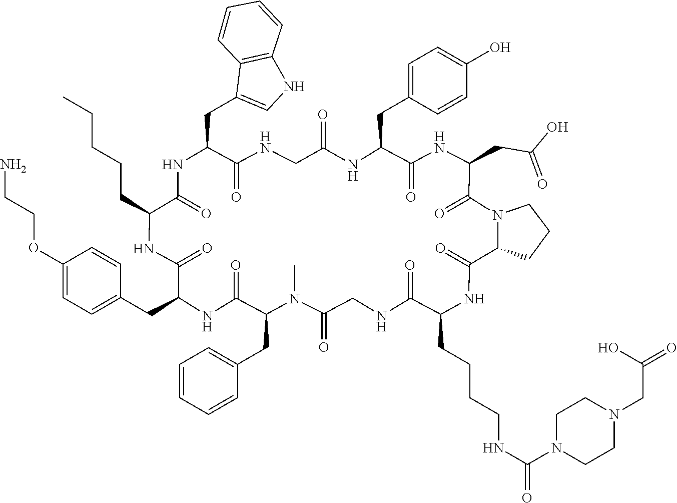 [SMILES: CCCCC[C@@H]1NC(=O)[C@H](Cc2ccc(OCCN)cc2)NC(=O)[C@H](Cc2ccccc2)N(C)C(=O)CNC(=O)[C@H](CCCCNC(=O)N2CCN(CC(=O)O)CC2)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-2 t_{R} = 6.94 min [M + H]⁺ = 1468.7 |
| | 2-(4-((4-((3S,9S,12S,15S,18S,24S,27S,32aR)-18-((1H-indol-3-yl)methyl)-12-(4-(2- | |
| | aminoethoxy)benzyl)-9-benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-3- | |
| | yl)butyl)carbamoyl)piperazin-1-yl)acetic acid TFA salt | |
| A47 | 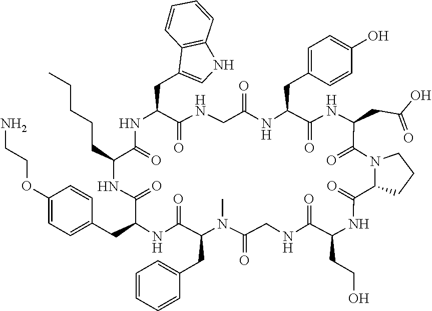 [SMILES: CCCCC[C@@H]1NC(=O)[C@H](Cc2ccc(OCCN)cc2)NC(=O)[C@H](Cc2ccccc2)N(C)C(=O)CNC(=O)[C@H](CCO)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-2 t_{R} = 7.49 min [M + H]⁺ = 1271.6 |
| | 2-((3S,9S,12S,15S,18S,24S,27S,32aR)-18-((1H-indol-3-yl)methyl)-12-(4-(2- | |
| | aminoethoxy)benzyl)-9-benzyl-24-(4-hydroxybenzyl)-3-(2-hydroxyethyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A48 | 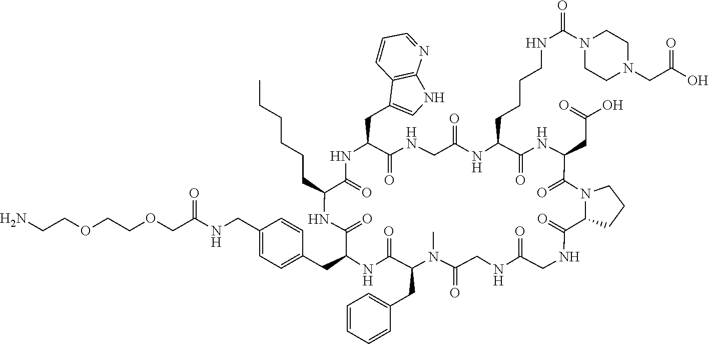 [SMILES: CCCCCC[C@@H]1NC(=O)[C@H](Cc2ccc(CNC(=O)COCCOCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-1 t_{R} = 2.76 min [M + H]⁺ = 1492.8 |
| | 2-((9S,12S,15S,18S,24S,27S,32aR)-18-((1H-pyrrolo[2,3-b]pyridin-3-yl)methyl)-12- | |
| | (4-((2-(2-(2-aminoethoxy)ethoxy)acetamido)methyl)benzyl)-9-benzyl-24-(4-(4- | |
| | (carboxymethyl)piperazine-1-carboxamido)butyl)-15-hexyl-8-methyl- | |
| | 1,4,7,10,13,16,19,22,25,28-decaoxodotriacontahydropyrrolo[1,2- | |
| | a][1,4,7,10,13,16,19,22,25,28]decaazacyclotriacontin-27-yl)acetic acid TFA salt | |
| A49 | 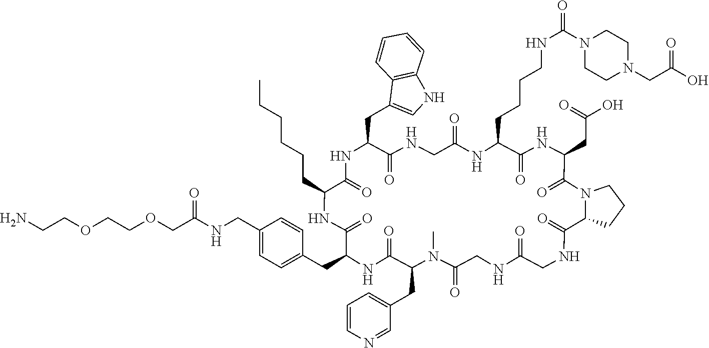 [SMILES: CCCCCC[C@@H]1NC(=O)[C@H](Cc2ccc(CNC(=O)COCCOCCN)cc2)NC(=O)[C@H](Cc2cccnc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 2.75 min [M + H]⁺ = 1492.8 |
| | 2-((9S,12S,15S,18S,24S,27S,32aR)-18-((1H-indol-3-yl)methyl)-12-(4-((2-(2-(2- | |
| | aminoethoxy)ethoxy)acetamido)methyl)benzyl)-24-(4-(4-(carboxymethyl)piperazine- | |
| | 1-carboxamido)butyl)-15-hexyl-8-methyl-1,4,7,10,13,16,19,22,25,28-decaoxo-9- | |
| | (pyridin-3-ylmethyl)dotriacontahydropyrrolo[1,2- | |
| | a][1,4,7,10,13,16,19,22,25,28]decaazacyclotriacontin-27-yl)acetic acid TFA salt | |
| A52 | 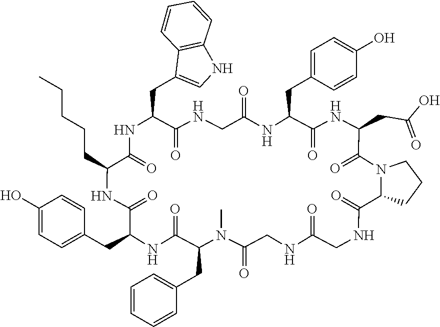 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 4.61 min [M + H]⁺ = 1184.4 |
| | 2-((9S,12S,15S,18S,24S,27S,32aR)-18-((1H-indol-3-yl)methyl)-9-benzyl-12,24-bis(4- | |
| | hydroxybenzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A53 | 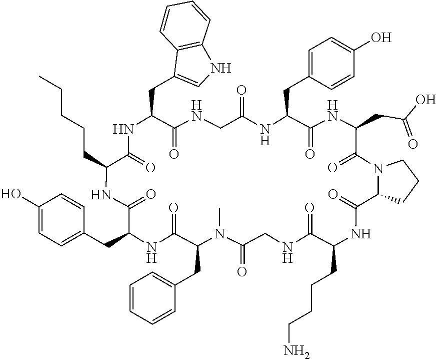 [SMILES: CCCCC[C@@H]1NC(=O)[C@H](Cc2ccc(O)cc2)NC(=O)[C@H](Cc2ccccc2)N(C)C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.77 min [M + H]⁺ = 1255.6 |
| | 2-((3S,9S,12S,15S,18S,24S,27S,32aR)-18-((1H-indol-3-yl)methyl)-3-(4-aminobutyl)- | |
| | 9-benzyl-12,24-bis(4-hydroxybenzyl)-8-methyl-1,4,7,10,13,16,19,22,25,28-decaoxo- | |
| | 15-pentyldotriacontahydropyrrolo[1,2- | |
| | a][1,4,7,10,13,16,19,22,25,28]decaazacyclotriacontin-27-yl)acetic acid TFA salt | |
| A54 | 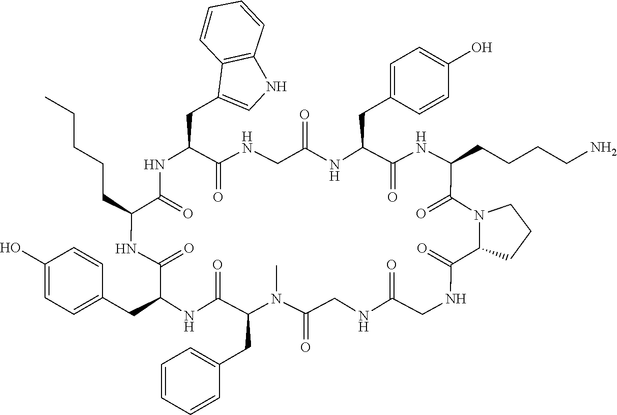 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CCCCN)NC(=O)[C@H](Cc2ccc(O)cc2)NC(=O)CNC(=O)[C@H](Cc2c[nH]c3ccccc23)NC1=O] | Method P-1 t_{R} = 3.73 min [M + H]⁺ = 1197.6 |
| | (9S,12S,15S,18S,24S,27S,32aR)-18-((1H-indol-3-yl)methyl)-27-(4-aminobutyl)-9- | |
| | benzyl-12,24-bis(4-hydroxybenzyl)-8-methyl-15-pentyldocosahydropyrrolo[1,2- | |
| | a][1,4,7,10,13,16,19,22,25,28]decaazacyclotriacontine-1,4,7,10,13,16,19,22,25,28- | |
| | decaone TFA salt | |
| A56 | 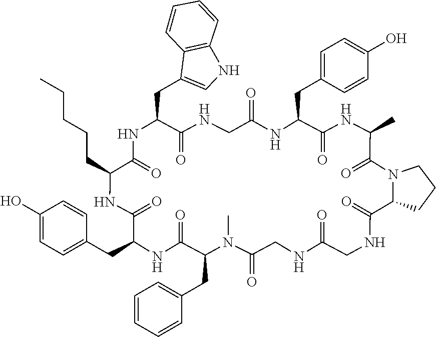 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](C)NC(=O)[C@H](Cc2ccc(O)cc2)NC(=O)CNC(=O)[C@H](Cc2c[nH]c3ccccc23)NC1=O] | Method P-1 t_{R} = 4.73 min [M + H]⁺ = 1140.6 |
| | (9S,12S,15S,18S,24S,27S,32aR)-18-((1H-indol-3-yl)methyl)-9-benzyl-12,24-bis(4- | |
| | hydroxybenzyl)-8,27-dimethyl-15-pentyldocosahydropyrrolo[1,2- | |
| | a][1,4,7,10,13,16,19,22,25,28]decaazacyclotriacontine-1,4,7,10,13,16,19,22,25,28- | |
| | decaone | |
| A57 | 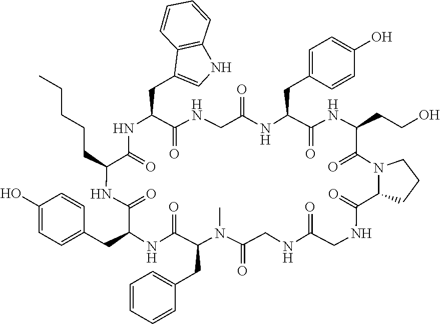 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CCO)NC(=O)[C@H](Cc2ccc(O)cc2)NC(=O)CNC(=O)[C@H](Cc2c[nH]c3ccccc23)NC1=O] | Method P-1 t_{R} = 4.61 min [M + H]⁺ = 1170.6 |
| | (9S,12S,15S,18S,24S,27S,32aR)-18-((1H-indol-3-yl)methyl)-9-benzyl-12,24-bis(4- | |
| | hydroxybenzyl)-27-(2-hydroxyethyl)-8-methyl-15-pentyldocosahydropyrrolo[1,2- | |
| | a][1,4,7,10,13,16,19,22,25,28]decaazacyclotriacontine-1,4,7,10,13,16,19,22,25,28- | |
| | decaone | |
| A58 | 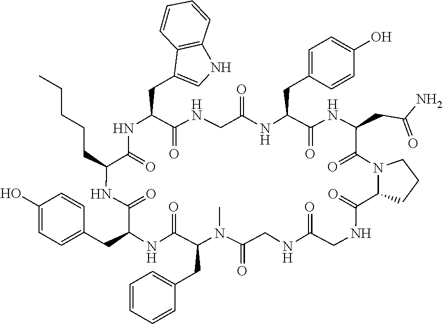 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(N)=O)NC(=O)[C@H](Cc2ccc(O)cc2)NC(=O)CNC(=O)[C@H](Cc2c[nH]c3ccccc23)NC1=O] | Method P-1 t_{R} = 4.51 min [M + H]⁺ = 1183.6 |
| | 2-((9S,12S,15S,18S,24S,27S,32aR)-18-((1H-indol-3-yl)methyl)-9-benzyl-12,24-bis(4- | |
| | hydroxybenzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetamide | |
| A59 | 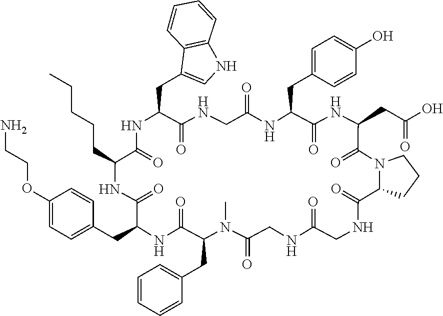 [SMILES: CCCCC[C@@H]1NC(=O)[C@H](Cc2ccc(OCCN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-7 t_{R} = 1.72 min [M + H]⁺ = 1227.6 |
| | 2-((9S,12S,15S,18S,24S,27S,32aR)-18-((1H-indol-3-yl)methyl)-12-(4-(2- | |
| | aminoethoxy)benzyl)-9-benzyl-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-27-yl)acetic acid TFA salt | |
| A60 | 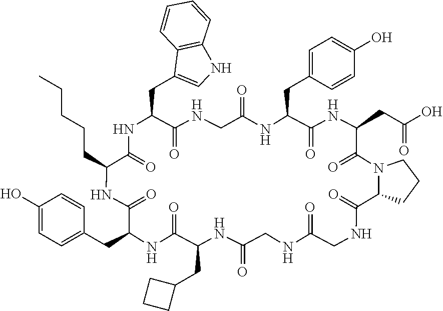 [SMILES: CCCCC[C@@H]1NC(=O)[C@H](Cc2ccc(O)cc2)NC(=O)[C@H](CC2CCC2)NC(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.52 min [M + H]⁺ = 1148.7 |
| | 2-((9S,12S,15S,18S,24S,27S,32aR)-18-((1H-indol-3-yl)methyl)-9-(cyclobutylmethyl)- | |
| | 12,24-bis(4-hydroxybenzyl)-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A61 | 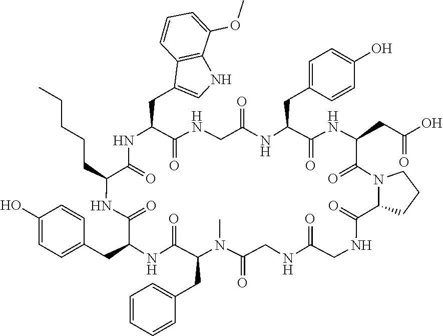 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3c(OC)cccc23)NC1=O] | Method P-5 t_{R} = 1.56 min [M + H]⁺ = 1214.7 |
| | 2-((9S,12S,15S,18S,24S,27S,32aR)-9-benzyl-12,24-bis(4-hydroxybenzyl)-18-((7- | |
| | methoxy-1H-indol-3-yl)methyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A62 | 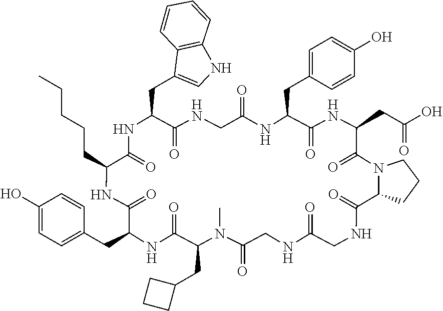 [SMILES: CCCCC[C@@H]1NC(=O)[C@H](Cc2ccc(O)cc2)NC(=O)[C@H](CC2CCC2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-6 t_{R} = 1.47 min [M + H]⁺ = 1162.6 |
| | 2-((9S,12S,15S,18S,24S,27S,32aR)-18-((1H-indol-3-yl)methyl)-9-(cyclobutylmethyl)- | |
| | 12,24-bis(4-hydroxybenzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A63 | 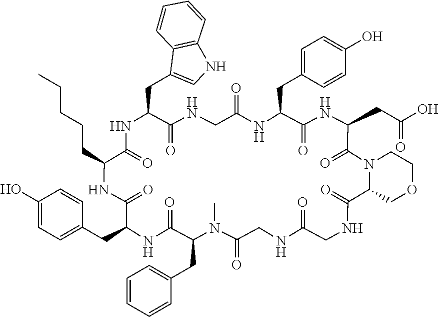 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2COCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-6 t_{R} = 1.45 min [M + H]⁺ = 1200.5 |
| | 2-((7S,10S,16S,19S,22S,25S,33aR)-16-((1H-indol-3-yl)methyl)-25-benzyl-10,22- | |
| | bis(4-hydroxybenzyl)-26-methyl-6,9,12,15,18,21,24,27,30,33-decaoxo-19- | |
| | pentyldotriacontahydro-[1,4]oxazino[4,3- | |
| | a][1,4,7,10,13,16,19,22,25,28]decaazacyclotriacontin-7-yl)acetic acid | |
| A64 | 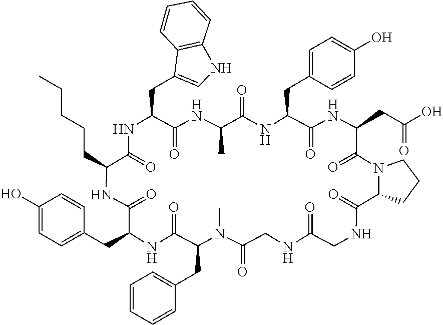 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)[C@@H](C)NC(=O)[C@H](Cc2c[nH]c3ccccc23)NC1=O] | Method P-5 t_{R} = 1.56 min [M + H]⁺ = 1198.7 |
| | 2-((9S,12S,15S,18S,21R,24S,27S,32aR)-18-((1H-indol-3-yl)methyl)-9-benzyl-12,24- | |
| | bis(4-hydroxybenzyl)-8,21-dimethyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A65 | 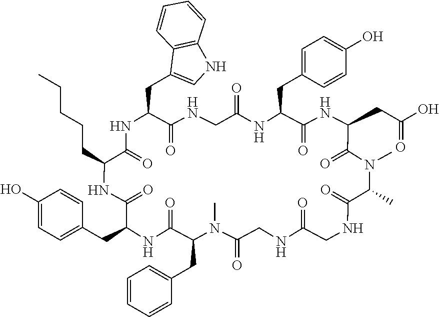 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@@H](C)N(C)C(=O)[C@H](CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.55 min [M + H]⁺ = 1172.7 |
| | 2-((2S,5R,14S,17S,20S,23S,29S)-23-((1H-indol-3-yl)methyl)-14-benzyl-17,29-bis(4- | |
| | hydroxybenzyl)-4,5,13-trimethyl-3,6,9,12,15,18,21,24,27,30-decaoxo-20-pentyl- | |
| | 1,4,7,10,13,16,19,22,25,28-decaazacyclotriacontan-2-yl)acetic acid | |
| A66 | 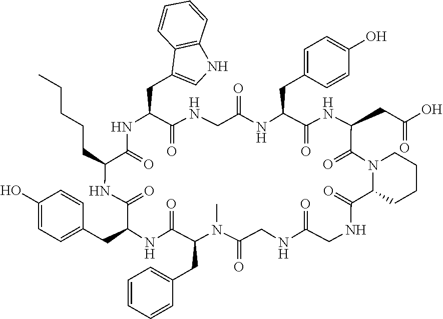 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-6 t_{R} = 1.48 min [M + H]⁺ = 1198.6 |
| | 2-((9S,12S,15S,18S,24S,27S,33aR)-18-((1H-indol-3-yl)methyl)-9-benzyl-12,24-bis(4- | |
| | hydroxybenzyl)-8-methyl-1,4,7,10,13,16,19,22,25,28-decaoxo-15- | |
| | pentyldotriacontahydro-2H-pyrido[1,2- | |
| | a][1,4,7,10,13,16,19,22,25,28]decaazacyclotriacontin-27-yl)acetic acid | |
| A67 | 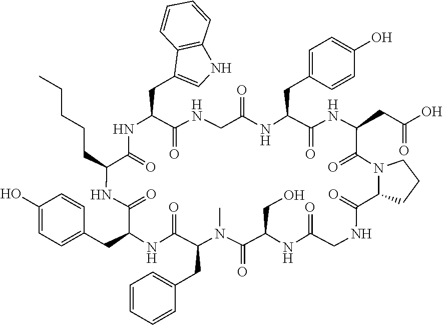 [SMILES: CCCCC[C@@H]1NC(=O)[C@H](Cc2ccc(O)cc2)NC(=O)[C@H](Cc2ccccc2)N(C)C(=O)[C@@H](CO)NC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.54 min [M + H]⁺ = 1214.7 |
| | 2-((6R,9S,12S,15S,18S,24S,27S,32aR)-18-((1H-indol-3-yl)methyl)-9-benzyl-12,24- | |
| | bis(4-hydroxybenzyl)-6-(hydroxymethyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid | |
| A68 | 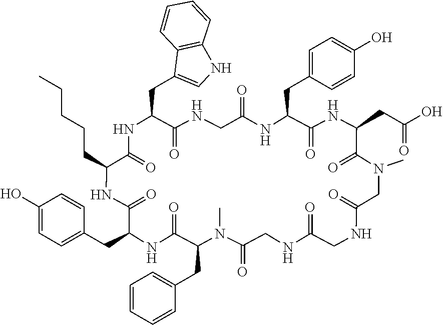 [SMILES: CCCCC[C@@H]1NC(=O)[C@H](Cc2ccc(O)cc2)NC(=O)[C@H](Cc2ccccc2)N(C)C(=O)CNC(=O)CNC(=O)CN(C)C(=O)[C@H](CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.54 min [M + H]⁺ = 1158.7 |
| | 2-((2S,14S,17S,20S,23S,29S)-23-((1H-indol-3-yl)methyl)-14-benzyl-17,29-bis(4- | |
| | hydroxybenzyl)-4,13-dimethyl-3,6,9,12,15,18,21,24,27,30-decaoxo-20-pentyl- | |
| | 1,4,7,10,13,16,19,22,25,28-decaazacyclotriacontan-2-yl)acetic acid | |
| A69 | 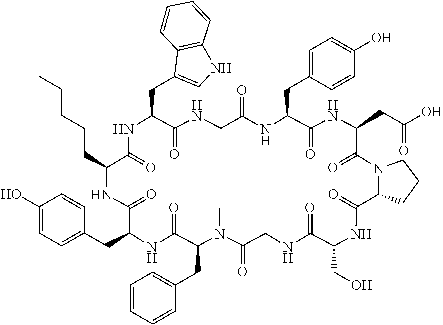 [SMILES: CCCCC[C@@H]1NC(=O)[C@H](Cc2ccc(O)cc2)NC(=O)[C@H](Cc2ccccc2)N(C)C(=O)CNC(=O)[C@@H](CO)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.54 min [M + H]⁺ = 1214.7 |
| | 2-((3R,9S,12S,15S,18S,24S,27S,32aR)-18-((1H-indol-3-yl)methyl)-9-benzyl-12,24- | |
| | bis(4-hydroxybenzyl)-3-(hydroxymethyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-27-yl)acetic acid | |
| A70 | 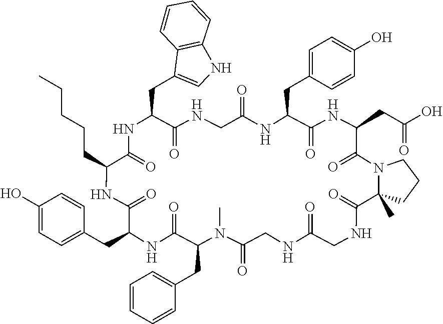 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@@]2(C)CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.56 min [M + H]⁺ = 1198.7 |
| | 2-((9S,12S,15S,18S,24S,27S,32aR)-18-((1H-indol-3-yl)methyl)-9-benzyl-12,24-bis(4- | |
| | hydroxybenzyl)-8,32a-dimethyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |
| A71 | 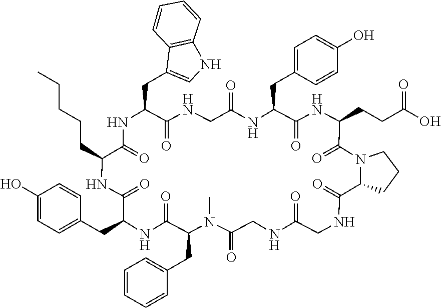 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CCC(=O)O)NC(=O)[C@H](Cc2ccc(O)cc2)NC(=O)CNC(=O)[C@H](Cc2c[nH]c3ccccc23)NC1=O] | Method P-5 t_{R} = 1.55 min [M + H]⁺ = 1198.7 |
| | 3-((9S,12S,15S,18S,24S,27S,32aR)-18-((1H-indol-3-yl)methyl)-9-benzyl-12,24-bis(4- | |
| | hydroxybenzyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)propanoic acid | |
| A72 | 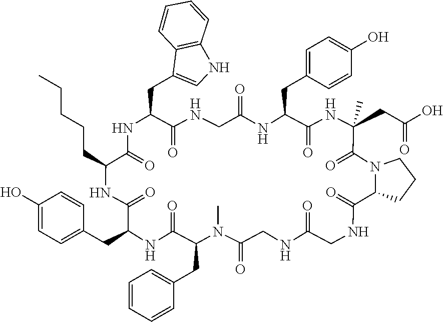 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@](C)(CC(=O)O)NC(=O)[C@H](Cc2ccc(O)cc2)NC(=O)CNC(=O)[C@H](Cc2c[nH]c3ccccc23)NC1=O] | Method P-5 t_{R} = 1.55 min [M + H]⁺ = 1198.7 |
| | 2-((9S,12S,15S,18S,24S,27S,32aR)-18-((1H-indol-3-yl)methyl)-9-benzyl-12,24-bis(4- | |
| | hydroxybenzyl)-8,27-dimethyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-27-yl)acetic acid | |

### 2.4 Examples Including Post-Modifications of Side Chains

### 2.4.1 Synthesis of 2-((9S,12S,15S,18S,24S,27S,32aR)-18-((1H-indol-3-yl)methyl)-9-benzyl-24-(2-(4-(carboxymethyl)piperazine-1-carboxamido)ethyl)-12-(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-27-yl)acetic acid TFA salt (Example A5)

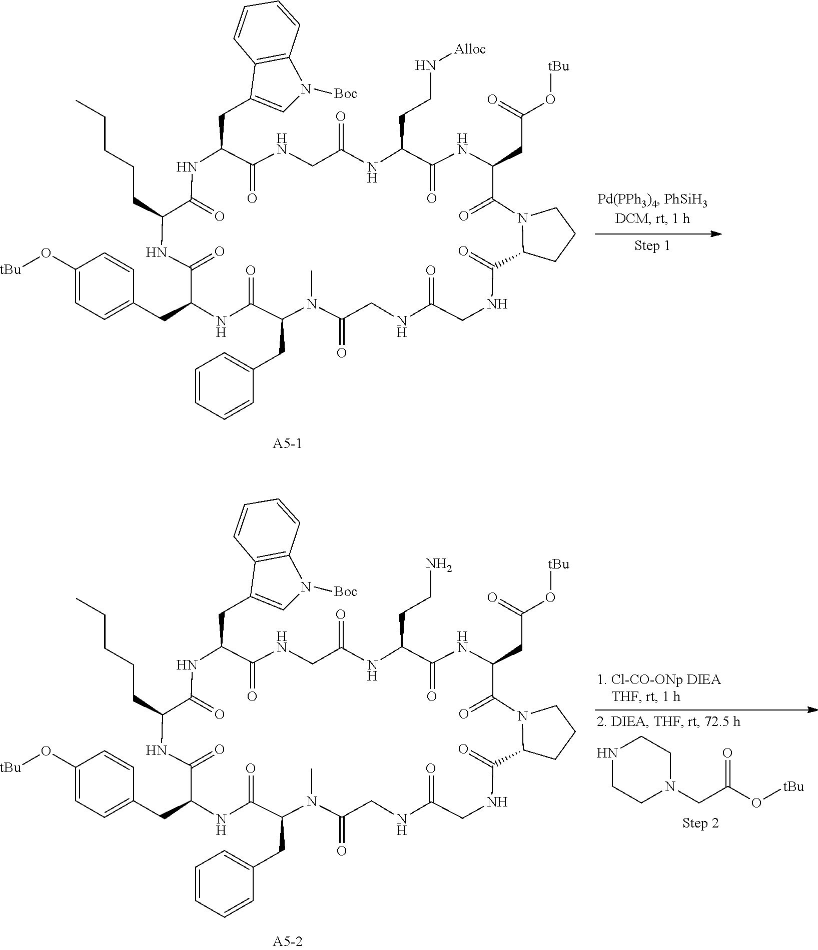

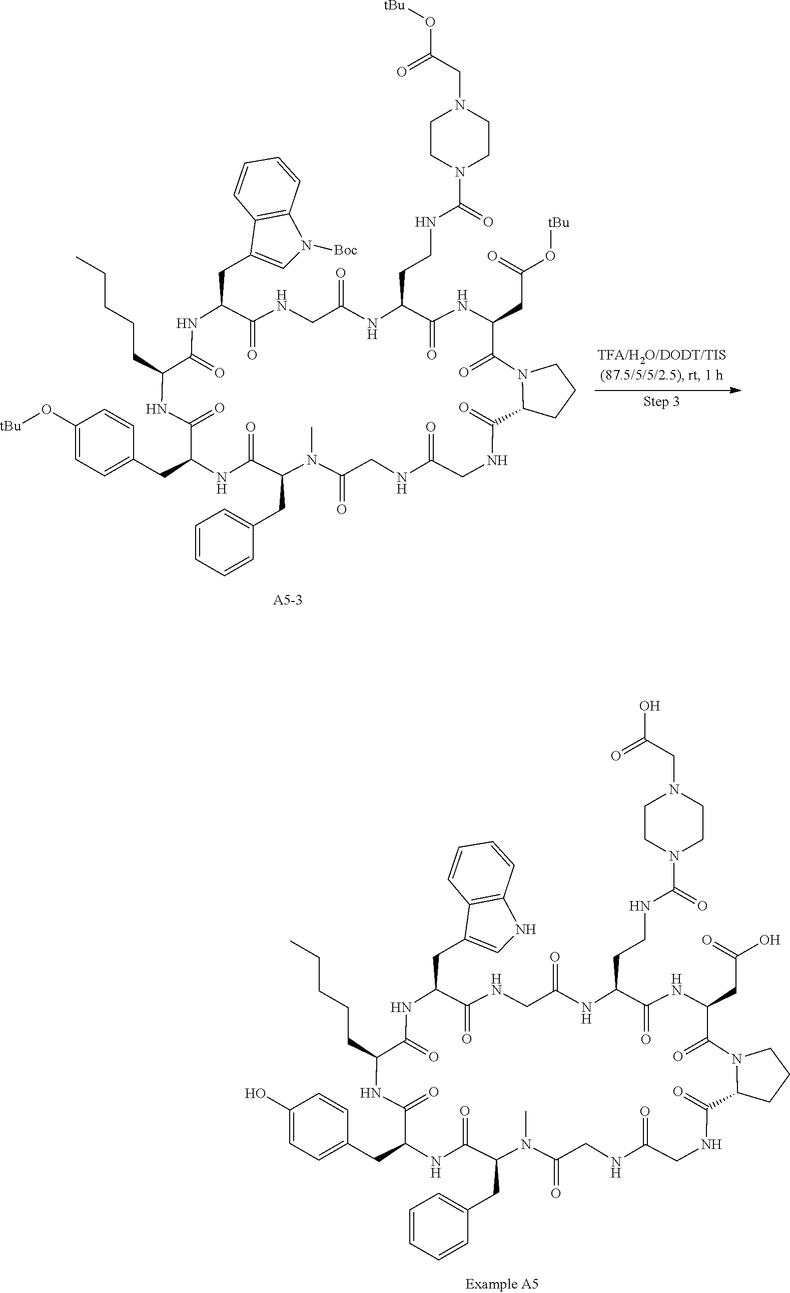 [SMILES: CCCCC[C@@H]1NC(=O)[C@H](Cc2ccc(OC(C)(C)C)cc2)NC(=O)[C@H](Cc2ccccc2)N(C)C(=O)CNC(=O)CNC(=O)[C@H]2CCCN2C(=O)[C@H](CC(=O)OC(C)(C)C)NC(=O)[C@H](CCNC(=O)N2CCN(CC(=O)OC(C)(C)C)CC2)NC(=O)CNC(=O)[C@H](Cc2cn(C(=O)OC(C)(C)C)c3ccccc23)NC1=O ; CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCNC(=O)N2CCN(CC(=O)O)CC2)NC(=O)CNC(=O)[C@H](Cc2c[nH]c3ccccc23)NC1=O]

### Scheme 2.4.1. Synthesis of Example A5

### Step 1. A5-2

The peptide A5-1 (48.1 mg, 32.2 μmol) and phenylsilane (87.2 mg, 99.3 μL, 25 eq., 806 μmol) were dissolved in DCM (3.5 mL). The reaction vial was sealed and several times evacuated and purged with nitrogen. A clear solution of tetrakis(triphenylphosphin)palladium (3.72 mg, 0.1 eq., 3.22 μmol) in DCM (0.5 mL) was added at rt. The reaction was stirred for 1 h at rt, then concentrated to dryness in vacuo. The product was isolated by preparative reversed-phase HPLC (Column: SunFire C18 OBD Prep Column, 100 Å, 5 μm, 30 mm×100 mm; Eluent A: H₂O+0.1% TFA and eluent B: ACN). Pure fractions were combined and lyophilized to afford A5-2 (29.7 mg, 19 μmol, 59% yield, TFA salt) as a white solid. LCMS method P-1, t_{R}=6.07 min, [M+H]⁺=1333.7.

### Step 2. A5-3

Step 2-1: A clear solution of the peptide A5-2 (29.7 mg, 93% Wt, 19.1 μmol, TFA salt), DIEA (11.1 mg, 15.0 μL, 4.5 eq., 85.9 μmol) and 4-nitrophenyl chloroformate (7.69 mg, 2 eq., 38.2 μmol, CAS 7693-46-1) in THF (5 mL) was stirred at rt for 1 h.

Step 2-2: Then a solution of tert-butyl 2-(piperazin-1-yl)acetate (19.1 mg, 5 eq., 95.4 μmol) and DIEA (12.3 mg, 16.6 μL, 5 eq., 95.4 μmol) in THF (0.5 mL) was added and the reaction mixture was stirred at rt for 72.5 h. The reaction mixture was concentrated to dryness in vacuo.

### Step 3. Example A5

The residue was dissolved in TFA/H₂O/DODT/TIS (87.5/5/5/2.5) (7 mL) and stirred for 1 h at rt. The clear solution was then poured onto cold methyl tert-butyl ether/heptanes (1/1) (40 mL), giving a precipitate. The suspension was centrifuged and the solvent was decanted. The residue was washed with cold methyl tert-butyl ether/heptanes (1/1) (2×15 mL) and the mixture was centrifuged. The solvent was decanted and the residue was dried on high vacuum for 0.5 h at 40° C. The product was isolated by preparative reversed-phase HPLC (Column: XSelect Peptide CSH C18 OBD Prep Column, 130 Å, 5 μm, 30 mm×250 mm; Eluent A: H₂O+0.1% TFA and eluent B: ACN). Pure fractions were combined and lyophilized to afford Example A5 (11.8 mg, 8.1 μmol, 43% yield, TFA salt) as a white solid. LCMS method P-1, t_{R}=3.97 min, [M+H]⁺=1291.6.

The compounds in Table 2.11 were synthesized in analogy to Example A5.

TABLE 2.11
Example A4 was synthesized in analogy to Example A5 starting from A4-1

| Example | | |
|---|---|---|
| No. | Structure/Example Name | LCMS |
| A4 | 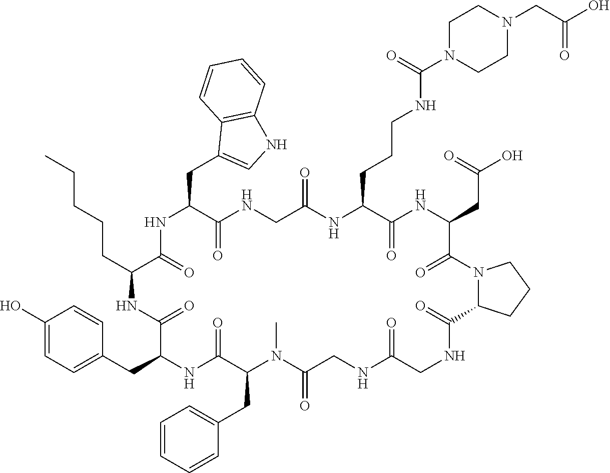 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCNC(=O)N2CCN(CC(=O)O)CC2)NC(=O)CNC(=O)[C@H](Cc2c[nH]c3ccccc23)NC1=O] | Method P-1 t_{R} = 3.97 min [M + H]⁺ = 1305.6 |

2-((9S,12S,15S,18S,24S,27S,32aR)-18-((1H-indol-3-yl)methyl)-9-benzyl-24-(3-(4-
(carboxymethyl)piperazine-1-carboxamido)propyl)-12-(4-hydroxybenzyl)-8-
methyl-1,4,7,10,13,16,19,22,25,28-decaoxo-15-
pentyldotriacontahydropyrrolo[1,2-
a][1,4,7,10,13,16,19,22,25,28]decaazacyclotriacontin-27-yl)acetic acid TFA salt

### 2.4.2 Synthesis of 2-((9S,12S,15S,18S,24S,27S,32aR)-18-((1H-indol-3-yl)methyl)-12-(4-(acetamidomethyl)benzyl)-9-benzyl-24-(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-27-yl)acetic acid (Example A28)

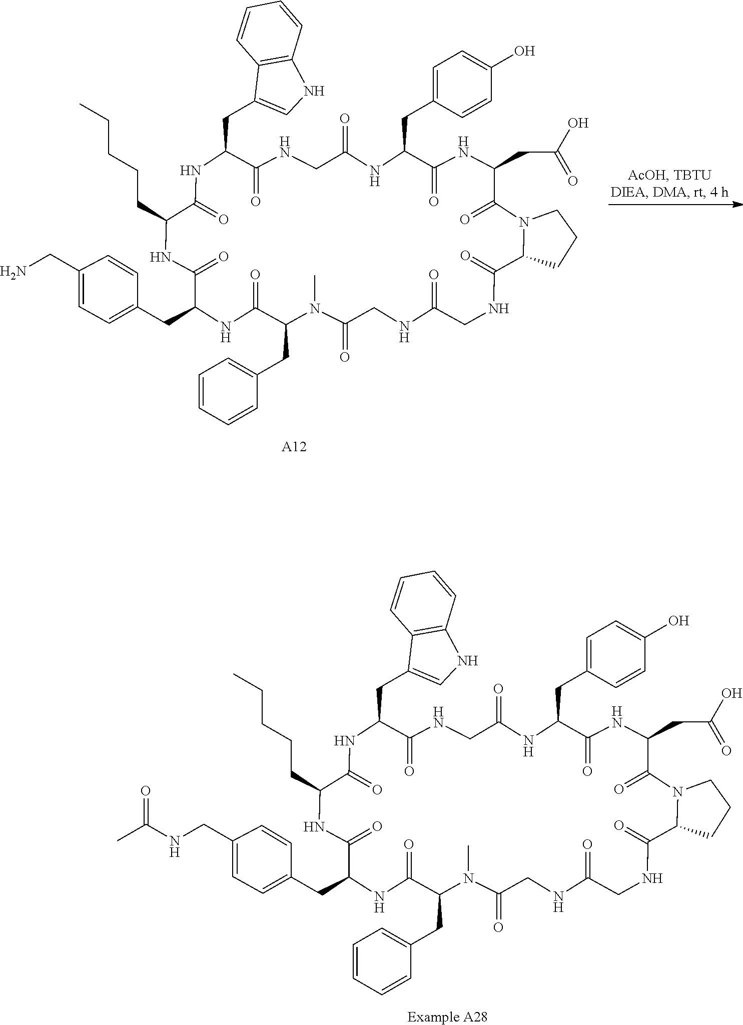 [SMILES: CCCCC[C@@H]1NC(=O)[C@H](Cc2ccc(CN)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O ; "A12" ; CCCCC[C@@H]1NC(=O)[C@H](Cc2ccc(CNC(C)=O)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O ; "Example A28"]

### Scheme 2.4.2. Synthesis of Example A28

To a clear solution of the peptide A12 (12.6 mg, 9.6 μmol, TFA salt) in DMA (0.5 mL) was added a solution of acetic acid (634.7 μg, 0.60 μL, 1.1 eq., 10.57 μmol), TBTU (3.39 mg, 1.1 eq., 10.57 μmol) and DIEA (3.10 mg, 4.18 μL, 2.5 eq., 24.0 μmol) in DMA (1.0 mL). The reaction mixture was stirred for 4 h at rt. The product was isolated by preparative HPLC (Column: XSelect Peptide CSH C18 OBD Prep Column, 130 Å, 5 μm, 30 mm×250 mm; Eluent A: H₂O+0.1% TFA and eluent B: ACN). Pure fractions were combined and lyophilized to afford Example A28 (9.9 mg, 7.9 μmol, 82% yield) as a white solid. LCMS method P-1, t_{R}=4.62 min, [M+H]⁺=1239.6.

Example A55 was synthesized starting from Example A54 following a similar strategy as for Example A28 using N-acetoxysuccinimide (instead of AcOH, TBTU and DIEA).

TABLE 2.12
Example synthesized in analogy to Example A28

| Example | | |
|---|---|---|
| No. | Structure/Example Name | LCMS |
| A55 | 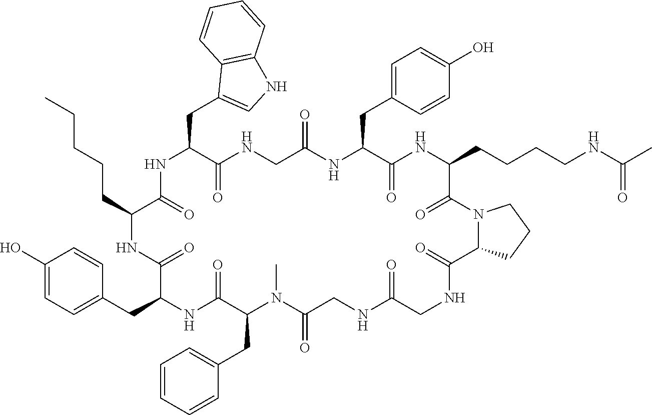 [SMILES: CCCCC[C@@H]1NC(=O)[C@H](Cc2ccc(O)cc2)NC(=O)[C@H](Cc2ccccc2)N(C)C(=O)CNC(=O)CNC(=O)[C@H]2CCCN2C(=O)[C@H](CCCCNC(C)=O)NC(=O)[C@H](Cc2ccc(O)cc2)NC(=O)CNC(=O)[C@H](Cc2c[nH]c3ccccc23)NC1=O] | Method P-1 t_{R} = 4.64 min [M + H]⁺ = 1239.6 |

N-(4-((9S,12S,15S,18S,24S,27S,32aR)-18-((1H-indol-3-yl)methyl)-9-benzyl-12,24-
bis(4-hydroxybenzyl)-8-methyl-1,4,7,10,13,16,19,22,25,28-decaoxo-15-
pentyldotriacontahydropyrrolo[1,2-
a][1,4,7,10,13,16,19,22,25,28]decaazacyclotriacontin-27-yl)butyl)acetamide

### 2.4.3 Synthesis of 2-(4-((4-((6S,9S,15S,18S,21S,24S,29aS,34aR)-15-((1H-indol-3-yl)methyl)-21-(4-((2-(2-(2-aminoethoxy)ethoxy)acetamido)methyl)benzyl)-24-benzyl-6-(carboxymethyl)-25-methyl-5,8,11,14,17,20,23,26,29,34-decaoxo-18-(2-propoxyethyl)dotriacontahydro-1H,5H-dipyrrolo[1,2-a:1′,2′-b₁][1,4,7,10,13,16,19,22,25,28]decaazacyclotriacontin-9-yl)butyl)carbamoyl)piperazin-1-yl)acetic acid TFA salt (Example A39)

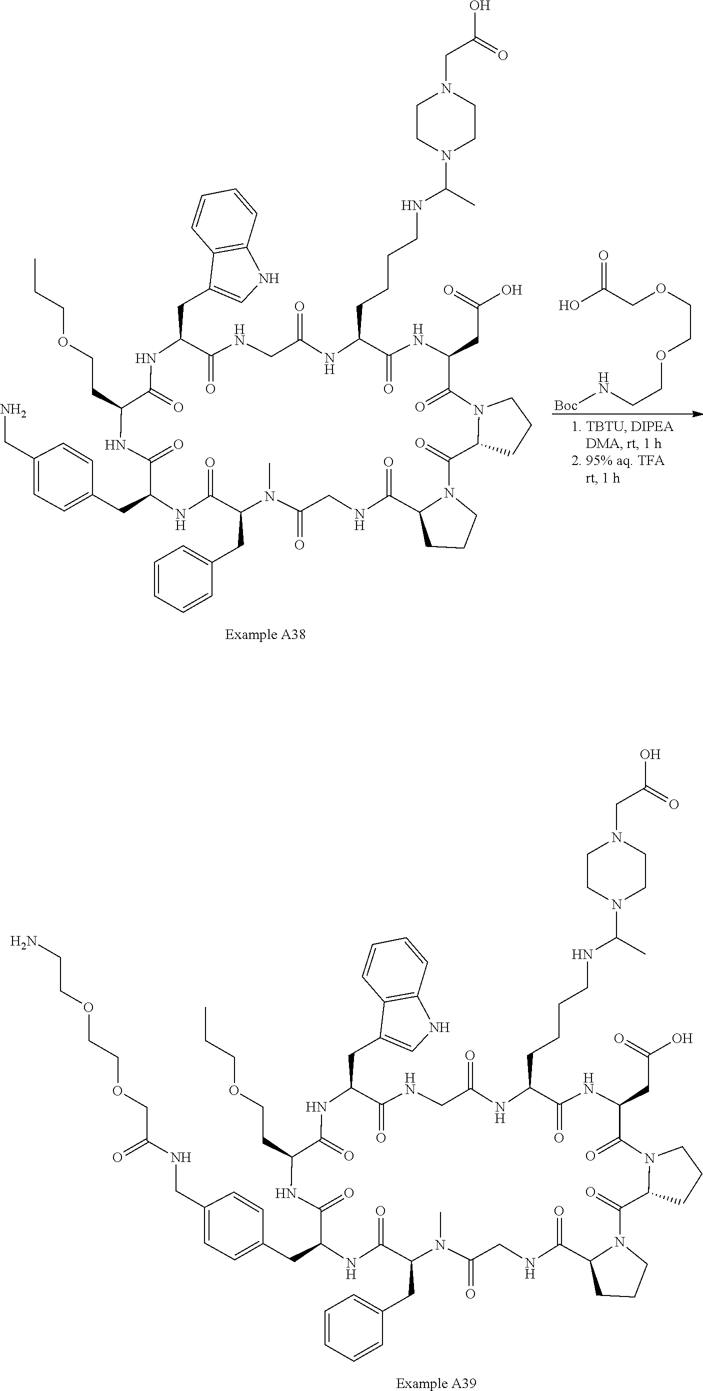 [SMILES: CCCOCC[C@@H]1NC(=O)[C@H](Cc2ccc(CN)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](CCCCNC(C)N2CCN(CC(=O)O)CC2)NC(=O)CNC(=O)[C@H](Cc2c[nH]c3ccccc23)NC1=O ; CC(C)(C)OC(=O)NCCOCCOCC(=O)O ; "Example A38" ; CCCOCC[C@@H]1NC(=O)[C@H](Cc2ccc(CNC(=O)COCCOCCN)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](CCCCNC(C)N2CCN(CC(=O)O)CC2)NC(=O)CNC(=O)[C@H](Cc2c[nH]c3ccccc23)NC1=O ; "Example A39"]

### Scheme 2.4.3. Synthesis of Example A39

To a clear solution of the Example A38 (80.7 mg, 47.42 μmol, TFA salt) in DMA (4.0 mL) was added DIEA (7.66 mg, 10.3 μL, 1.25 eq., 59.28 μmol). Then a solution of 2,2-dimethyl-4-oxo-3,8,11-trioxa-5-azatridecan-13-oic acid (13.73 mg, 1.1 eq., 52.16 μmol, CAS 108466-89-3), TBTU (16.75 mg, 1.1 eq., 52.16 μmol) and DIEA (7.66 mg, 10.3 μL, 1.25 eq., 59.28 μmol) in DMA (1.0 mL) was added. The reaction mixture was stirred for 1 h at rt, then concentrated to dryness in vacuo. The residue was dissolved in 95% aq. TFA (5 mL). The reaction was stirred for 1 h at rt and concentrated in vacuo. The product was isolated by preparative HPLC (Column: XSelect Peptide CSH C18 OBD Prep Column, 130 Å, 5 μm, 30 mm×250 mm; Eluent A: H₂O+0.1% TFA and eluent B: ACN). Pure fractions were combined and lyophilized to afford Example A39 (42.2 mg, 23.9 μmol, 50% yield, TFA salt) as a white solid. LCMS method P-1, t_{R}=3.15 min, [M+H]⁺=1533.8.

### 2.4.4 Synthesis of 2-((3S,9S,12S,15S,18S,24S,27S,32aR)-18-((1H-indol-3-yl)methyl)-3-(4-aminobutyl)-9-benzyl-15-(3-((S)-2-cyano-4,4-difluoropyrrolidin-1-yl)-3-oxopropyl)-12,24-bis(4-hydroxybenzyl)-8-methyl-1,4,7,10,13,16,19,22,25,28-decaoxodotriacontahydropyrrolo[1,2-a][1,4,7,10,13,16,19,22,25,28]decaazacyclotriacontin-27-yl)acetic acid TFA salt (Example A29)

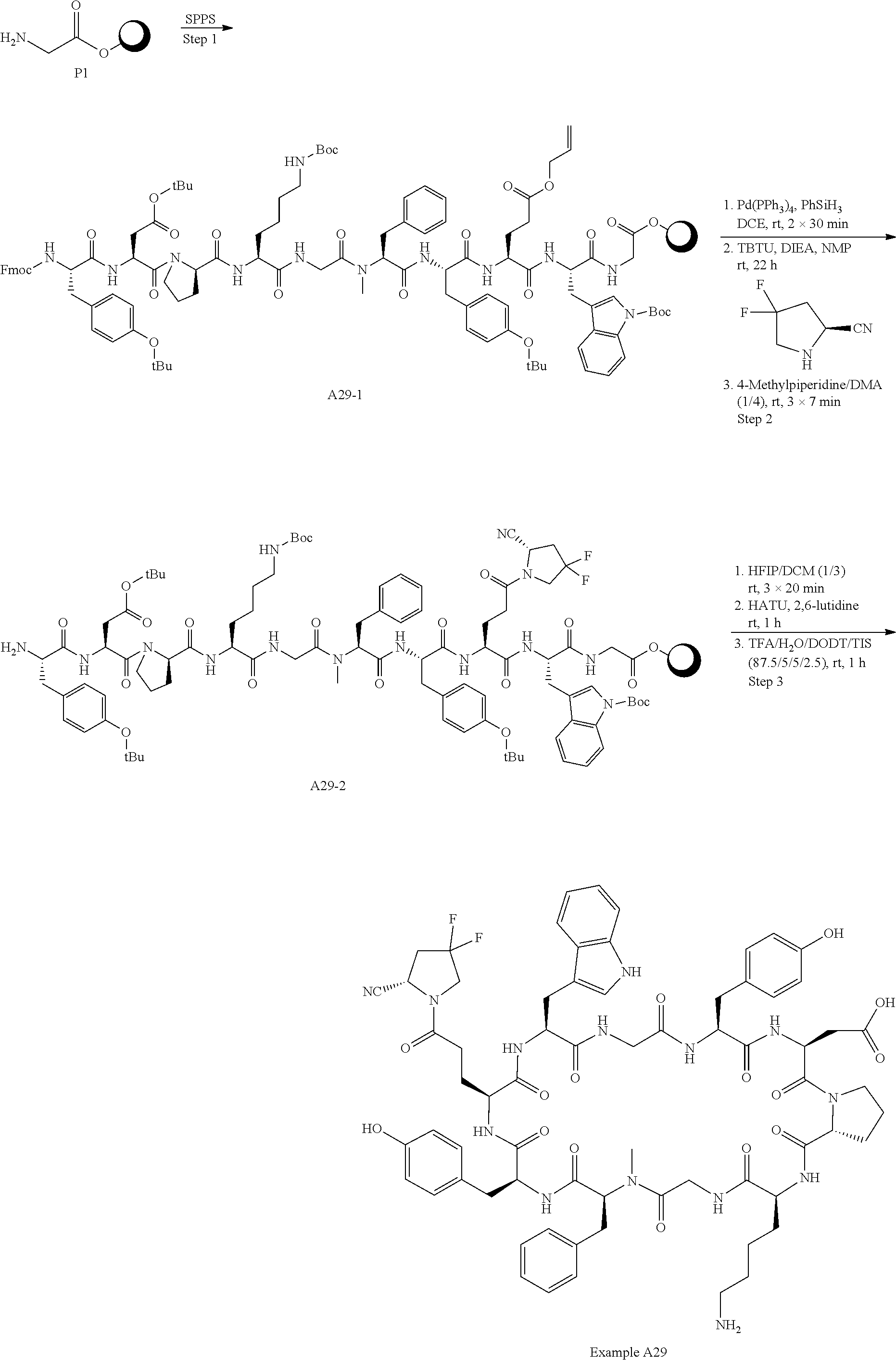

### Scheme 2.4.4. Synthesis of Example A29

### Step 1. A29-1

The assembly of the linear peptide was done on a Symphony X from Gyros Protein Technologies at rt using preloaded resin P1 (175 mg, 100 μmol, 0.572 mmol/g) (corresponding to A⁷-resin). The amino acids were coupled with a set of different methods.

### Method A

Fmoc-amino acid (0.2 M solution in NMP, 3 eq.), HATU (0.3 M solution in NMP, 3 eq.), DIEA (0.33 M solution in NMP, 3.3 eq), addition by synthesizer.

### Method B

Fmoc-amino acid (0.2 M solution in NMP, 3 eq.), PyOxim (0.3 M solution in NMP, 3 eq.), DIEA (0.33 M solution in NMP, 6.6 eq.), addition by synthesizer.

### Method C (Manual Addition)

Fmoc-amino acid (3 eq.) and HATU (3 eq.) were dissolved in NMP (3 mL) and DIEA (3.3 eq) was added. After a preactivation time of 5 min the solution was added to the resin.

The Fmoc removal was performed using a solution of 4-methylpiperidine/DMA (1/4). Capping was performed using Ac₂O/pyridine/DMA (1/1/8). After the assembly of the linear peptide A29-1 the resin was washed with DMA (5×) and DCM (5×). The coupling methods and times are summarized in Table 2.13.

TABLE 2.13

| Synthesis | Amino acid | | | |
|---|---|---|---|---|
| cycle | residue* | Amino acid | Time | Method |
| 1 | A⁶ | Fmoc-Trp(Boc)-OH | 2 × 1 h | A |
| 2 | A⁵ | Fmoc-Glu(All)-OH | 1 × 3 h | C |
| 3 | A⁴ | Fmoc-Tyr(tBu)-OH | 2 × 1 h | A |
| 4 | A³ | Fmoc-N-Me-Phe-OH | 2 × 1 h | A |
| 5 | A² | Fmoc-Gly-OH | 2 × 3 h | B |
| 6 | A¹ | Fmoc-Lys(Boc)-OH | 2 × 1 h | A |
| 7 | A¹⁰ | Fmoc-D-Pro-OH | 2 × 1 h | A |
| 8 | A⁹ | Fmoc-Asp(tBu)-OH | 2 × 2 h | B |
| 9 | A⁸ | Fmoc-Tyr(tBu)-OH | 2 × 1 h | A |

*Refers to the corresponding amino acid residue of cyclic peptide, P, as found, e.g., in Formulae (I), (Ia), (Ib), (Ic), (Id), or in the Table of Example III.

### Step 2. A29-2

Step 2-1: A clear solution of phenylsilane (379 mg, 646 μL, 35 eq., 3.5 mmol) and tetrakis(triphenylphosphin)palladium (11.6 mg, 0.1 eq., 10 μmol) in DCE (2 mL) was added to the resin A29-1 (calculated with 100 μmol) at rt. The reaction was agitated with nitrogen for 0.5 h. The resin was drained and the procedure was repeated once. The resin was drained and washed with DMA (3×) and DCM (5×)

Step 2-2: To the resin was added a clear solution of (S)-4,4-difluoropyrrolidine-2-carbonitrile hydrochloride (21.1 mg, 1.25 eq., 125 μmol, CAS 869489-04-3), TBTU (40.1 mg, 1.25 eq., 125 μmol) and DIEA (35.5 mg, 47.9 μL, 2.75 eq., 275 μmol) in NMP (2 mL). The mixture was agitated by bubbling with nitrogen for 4 h at rt on the Symphony X peptide synthesizer. The coupling was repeated once with the same amounts for 18 h at rt. The resin was drained and washed with DMA (3×) and DCM (5×).

Step 2-3: For Fmoc removal the resin was treated repeatedly at rt with 4-methylpiperidine/DMA (1/4) (3×7 min, each time 3 mL), then washed with DMA (6×) and DCM (5×) to afford A29-2.

### Step 3. Example A29

Step 3-1: To the resin A29-2 (calculated with 100 μmol) was added HFIP/DCM (1/3) (3 mL). The resin was shaken at rt for 20 min. The cleavage solution was filtered off and the procedure was repeated two times. The resin was washed with HFIP/DCM (1/3) (1 mL). The combined cleavage and washing solutions were concentrated to dryness in vacuo. The residue was treated with toluene and the resulting mixture was concentrated to dryness in vacuo. The residue was dried in high vacuum at 40° C. and obtained as an off-white solid. LCMS method P-1, t_{R}=6.34 min, [M+H]⁺=1757.9.

Step 3-2: To a clear solution of the residue from Step 3-1 in DCM (50 mL) at rt were added HATU (76.0 mg, 2 eq., 200 μmol), followed by 2,6-lutidine (268 mg, 290 μL, 25 eq., 2.50 mmol). The reaction was stirred at rt for 1 h, then concentrated to dryness in vacuo. LCMS method P-1, t_{R}=8.06 min, [M+H]⁺=1739.9.

Step 3-3: The residue from Step 3-2 was dissolved in TFA/H₂O/DODT/TIS (87.5/5/5/2.5) (7 mL) and the reaction was stirred at rt for 1 h. The clear solution was then poured onto cold methyl tert-butyl ether/heptanes (1/1) (40 mL), giving a precipitate. The suspension was centrifuged and the solvent was decanted. The residue was washed with cold methyl tert-butyl ether/heptanes (1/1) (2×15 mL), then dried in high vacuum at 40° C. for 30 min. The product was isolated by preparative HPLC (Column: CSH Peptide C18 OBD, 30×250 mm, 5 μm; Eluent A: Water+0.1% TFA and eluent B: ACN). Pure fractions were combined and lyophilized to afford Example A29 (16.5 mg, 11 μmol, 11% yield, TFA salt) as a white solid. LCMS method P-1, t_{R}=3.43 min, [M+H]=⁺1371.6.

The compounds in Table 2.14 were synthesized in analogy to Example A29.

TABLE 2.14
Example A30 was synthesized in analogy to Example A29 (Scheme 2.4.4)

| Example | | |
|---|---|---|
| No. | Structure/Example Name | LCMS |
| A30 | 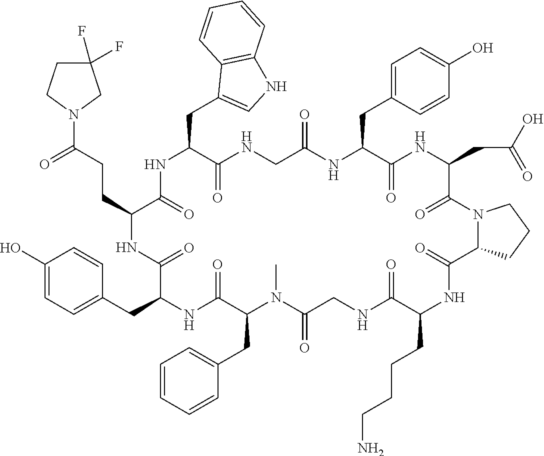 [SMILES: CN1C(=O)CNC(=O)[C@H](CCCCN)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC(=O)[C@H](CCC(=O)N2CCC(F)(F)C2)NC(=O)[C@H](Cc2ccc(O)cc2)NC(=O)[C@@H]1Cc1ccccc1] | Method P-1 t_{R} = 3.45 min [M + H]⁺ = 1346.6 |

2-((3S,9S,12S,15S,18S,24S,27S,32aR)-18-((1H-indol-3-yl)methyl)-3-(4-
aminobutyl)-9-benzyl-15-(3-(3,3-difluoropyrrolidin-1-yl)-3-oxopropyl)-12,24-
bis(4-hydroxybenzyl)-8-methyl-1,4,7,10,13,16,19,22,25,28-
decaoxodotriacontahydropyrrolo[1,2-
a][1,4,7,10,13,16,19,22,25,28]decaazacyclotriacontin-27-yl)acetic acid TFA salt

### 3 Coupling of the Chelators to the Parent Peptide (“B” Examples)

### 3.1 Coupling of DOTA

### 3.1.1 Synthesis of 2,2′,2″-(10-{1-[4-({(9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxodotriacontahydropyrrolo[1,2-a][1,4,7,10,13,16,19,22,25,28]decaazacyclotriacontin-12-yl}methyl)phenyl]-3,12-dioxo-5,8-dioxa-2,11-diazatridecan-13-yl}-1,4,7,10-tetraazacyclododecane-1,4,7-triyl)triacetic acid (Example B1)

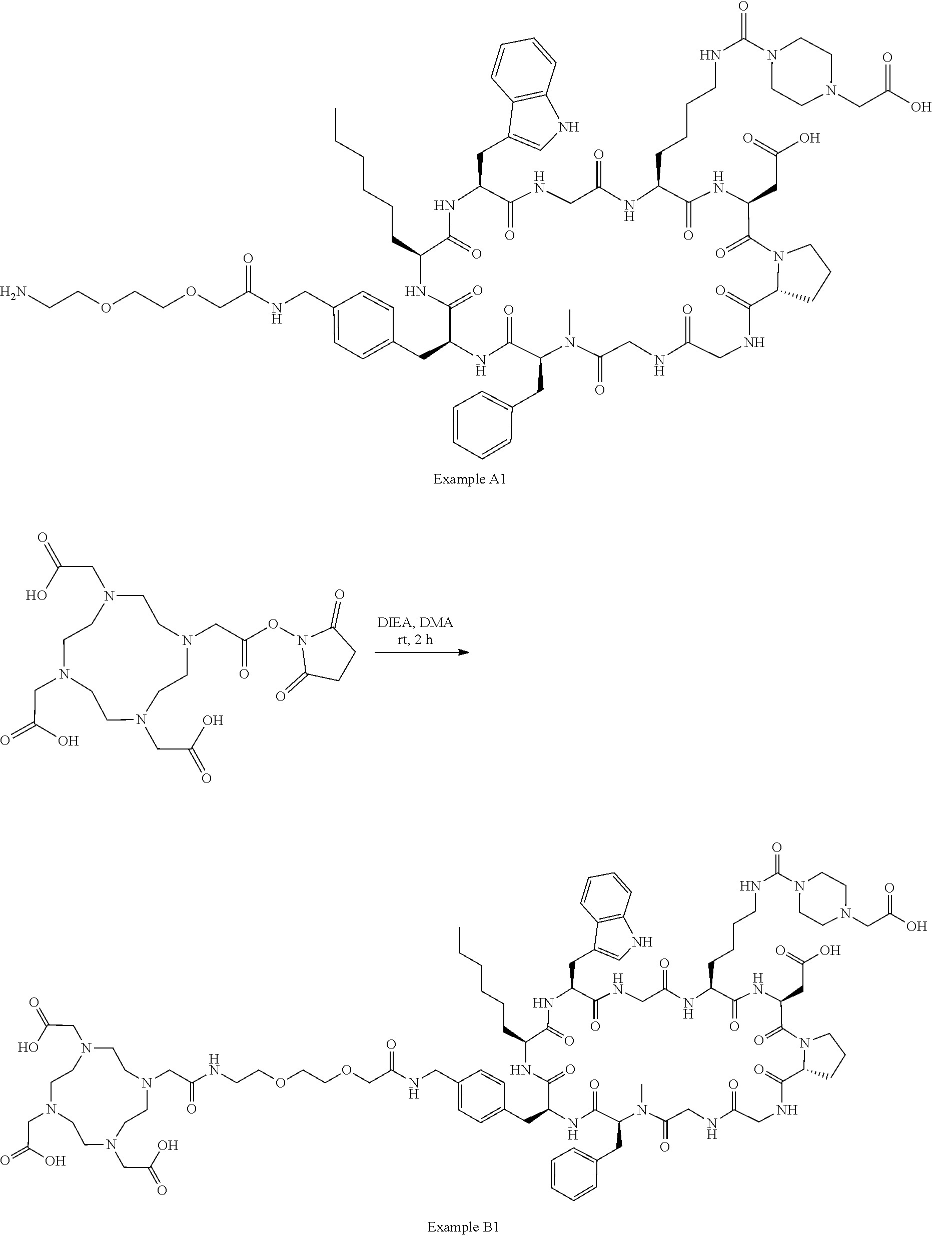

### Scheme 3.1.1. Synthesis of Example B1

To a clear colorless solution of Example A1 (72.0 mg, 95% Wt, 1 eq., 39.8 μmol, TFA salt) and DOTA-NHS ester (36.3 mg, 1.2 eq., 47.7 μmol, trifluoroacetate-hexafluorophosphate) in DMA (2.0 mL) at rt was added DIEA (51.4 mg, 69.3 μL, 10 eq., 398 μmol). The resulting clear solution was stirred at rt for 2 h. The product was isolated by preparative HPLC (Column: XBridge BEH C18 OBD Prep Column, 130 Å, 5 μm, 30 mm×100 mm; Eluent A: H₂O+0.08% NH₄CO₃ and eluent B: ACN). Pure fractions were combined and lyophilized to afford Example B1 (54.2 mg, 28 μmol, 70% yield) as a white solid. LCMS method P-1, t_{R}=3.43 min [M+H]⁺=1877.9.

The following examples (for structures see Table 3.1) were synthesized in analogy to Example B1:

Example B2 was synthesized from Example A2.

Example B3 was synthesized from Example A6.

Example B4 was synthesized from Example A7.

Example B5 was synthesized from Example A10.

Example B6 was synthesized from Example A8.

Example B7 was synthesized from Example A11.

Example B8 was synthesized from Example A9.

Example B13 was synthesized from Example A15.

Example B14 was synthesized from Example A16.

Example B15 was synthesized from Example A17.

Example B16 was synthesized from Example A27.

Example B17 was synthesized from Example A28.

Example B18 was synthesized from Example A35.

Example B19 was synthesized from Example A36.

Example B20 was synthesized from Example A37.

Example B21 was synthesized from Example A39.

Example B22 was synthesized from Example A53.

Example B23 was synthesized from Example A59.

Example B24 was synthesized from Example A45.

Example B25 was synthesized from Example A46.

Example B26 was synthesized from Example A47.

Example B27 was synthesized from Example A48.

Example B28 was synthesized from Example A49.

TABLE 3.1
Examples resulting from coupling with the DOTA chelator

| Ex- | | |
|---|---|---|
| ample | | |
| No. | Structure/Example Name | LCMS |
| B2 | 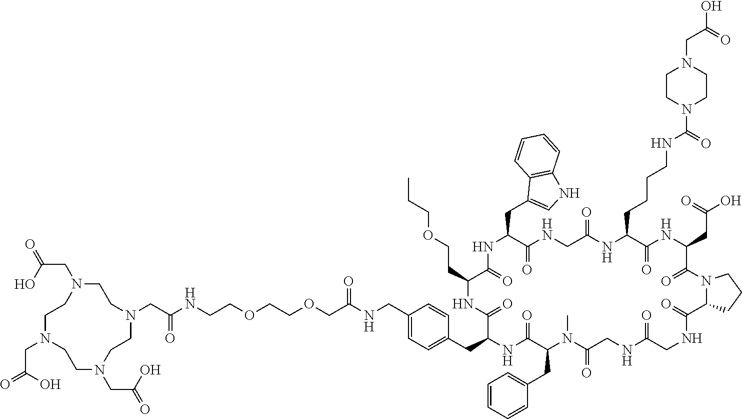 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.03 min [M + H]⁺ = 1880.1 |
| | 2,2′,2″-(10-(1-(4-(((9S,12S,15S,18S,24S,27S,32aR)-18-((1H-indol-3-yl)methyl)-9- | |
| | benzyl-27-(carboxymethyl)-24-(4-(4-(carboxymethyl)piperazine-1- | |
| | carboxamido)butyl)-8-methyl-1,4,7,10,13,16,19,22,25,28-decaoxo-15-(2- | |
| | propoxyethyl)dotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B3 | 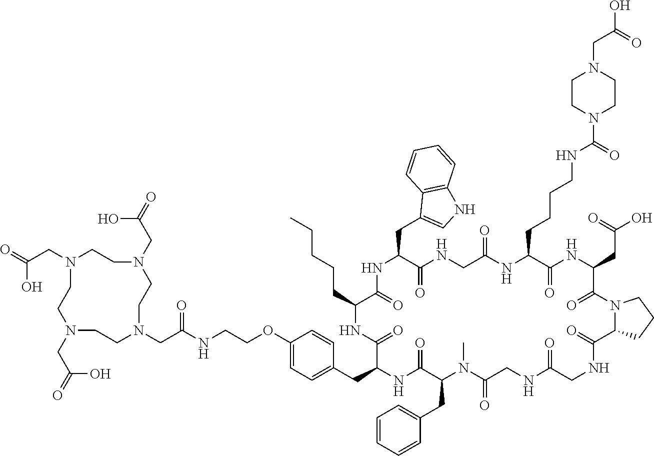 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-2 t_{R} = 6.01 min [M + H]⁺ = 1748.9 |
| | 2,2′,2″-(10-(2-((2-(4-(((9S,12S,15S,18S,24S,27S,32aR)-18-((1H-indol-3-yl)methyl)- | |
| | 9-benzyl-27-(carboxymethyl)-24-(4-(4-(carboxymethyl)piperazine-1- | |
| | carboxamido)butyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-12- | |
| | yl)methyl)phenoxy)ethyl)amino)-2-oxoethyl)-1,4,7,10-tetraazacyclododecane-1,4,7- | |
| | triyl)triacetic acid | |
| B4 | 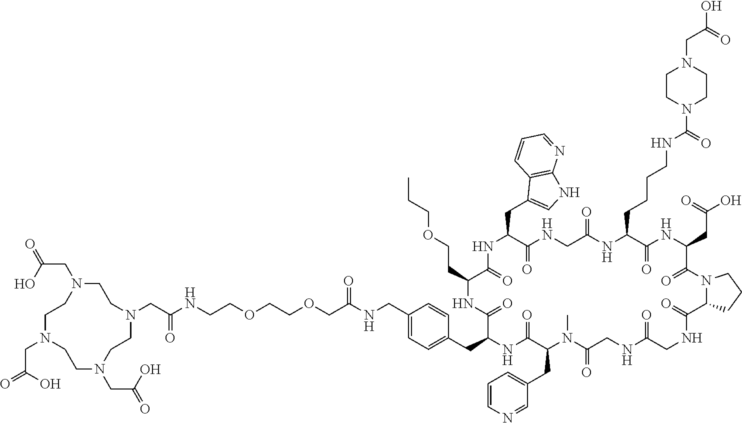 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2cccnc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 2.78 min [M + H]⁺ = 1881.9 |
| | 2,2′,2″-(10-(1-(4-(((9S,12S,15S,18S,24S,27S,32aR)-18-((1H-pyrrolo[2,3-b]pyridin-3- | |
| | yl)methyl)-27-(carboxymethyl)-24-(4-(4-(carboxymethyl)piperazine-1- | |
| | carboxamido)butyl)-8-methyl-1,4,7,10,13,16,19,22,25,28-decaoxo-15-(2- | |
| | propoxyethyl)-9-(pyridin-3-ylmethyl)dotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid | |
| B5 | 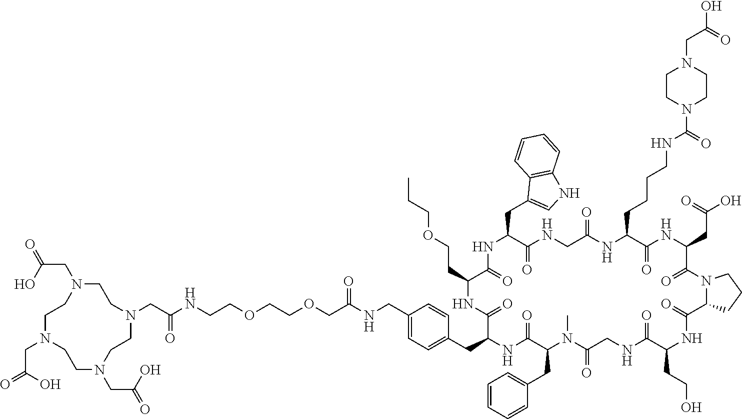 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)[C@H](CCO)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.00 min [M + H]⁺ = 1924.0 |
| | 2,2′,2″-(10-(1-(4-(((3S,9S,12S,15S,18S,24S,27S,32aR)-18-((1H-indol-3-yl)methyl)-9- | |
| | benzyl-27-(carboxymethyl)-24-(4-(4-(carboxymethyl)piperazine-1- | |
| | carboxamido)butyl)-3-(2-hydroxyethyl)-8-methyl-1,4,7,10,13,16,19,22,25,28- | |
| | decaoxo-15-(2-propoxyethyl)dotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid | |
| B6 | 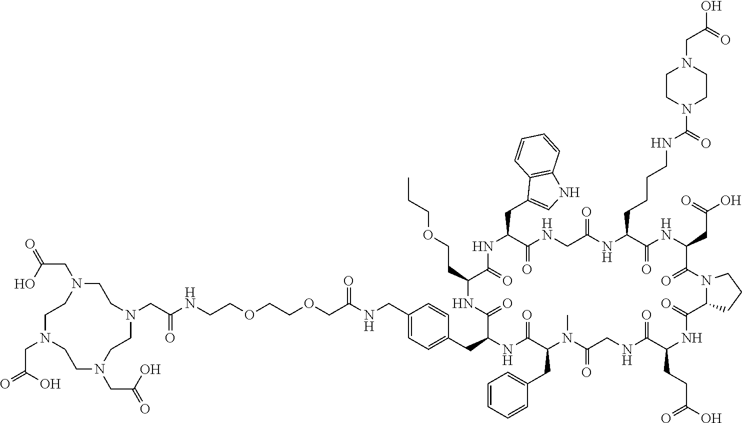 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)[C@H](CCC(=O)O)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-2 t_{R} = 5.95 min [M + H]⁺ = 1952.0 |
| | 2,2′,2″-(10-(1-(4-(((3S,9S,12S,15S,18S,24S,27S,32aR)-18-((1H-indol-3-yl)methyl)-9- | |
| | benzyl-3-(2-carboxyethyl)-27-(carboxymethyl)-24-(4-(4-(carboxymethyl)piperazine- | |
| | 1-carboxamido)butyl)-8-methyl-1,4,7,10,13,16,19,22,25,28-decaoxo-15-(2- | |
| | propoxyethyl)dotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid | |
| B7 | 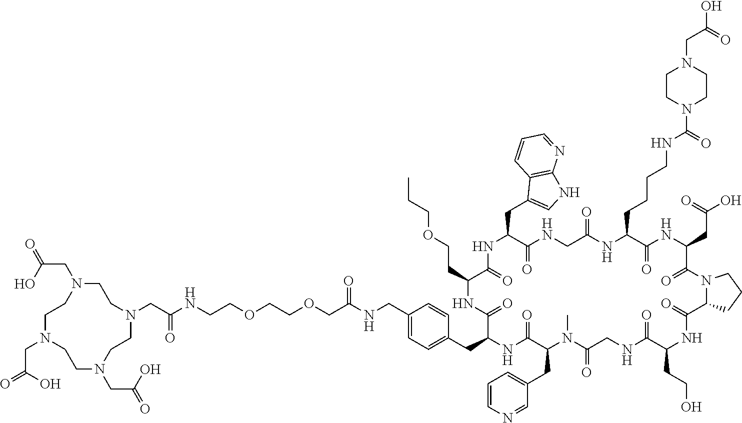 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2cccnc2)N(C)C(=O)CNC(=O)[C@H](CCO)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 2.69 min [M + 2H]²⁺ = 963.5 |
| | 2,2′,2″-(10-(1-(4-(((3S,9S,12S,15S,18S,24S,27S,32aR)-18-((1H-pyrrolo[2,3- | |
| | b]pyridin-3-yl)methyl)-27-(carboxymethyl)-24-(4-(4-(carboxymethyl)piperazine-1- | |
| | carboxamido)butyl)-3-(2-hydroxyethyl)-8-methyl-1,4,7,10,13,16,19,22,25,28- | |
| | decaoxo-15-(2-propoxyethyl)-9-(pyridin-3-ylmethyl)dotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid | |
| B8 | 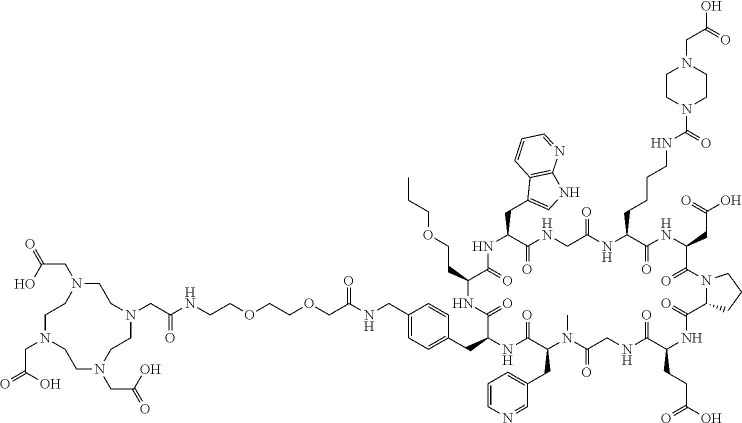 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2cccnc2)N(C)C(=O)CNC(=O)[C@H](CCC(=O)O)NC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-2 t_{R} = 2.58 min [M + 2H]²⁺ = 977.5 |
| | 2,2′,2″-(10-(1-(4-(((3S,9S,12S,15S,18S,24S,27S,32aR)-18-((1H-pyrrolo[2,3- | |
| | b]pyridin-3-yl)methyl)-3-(2-carboxyethyl)-27-(carboxymethyl)-24-(4-(4- | |
| | (carboxymethyl)piperazine-1-carboxamido)butyl)-8-methyl- | |
| | 1,4,7,10,13,16,19,22,25,28-decaoxo-15-(2-propoxyethyl)-9-(pyridin-3- | |
| | ylmethyl)dotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid | |
| B13 | 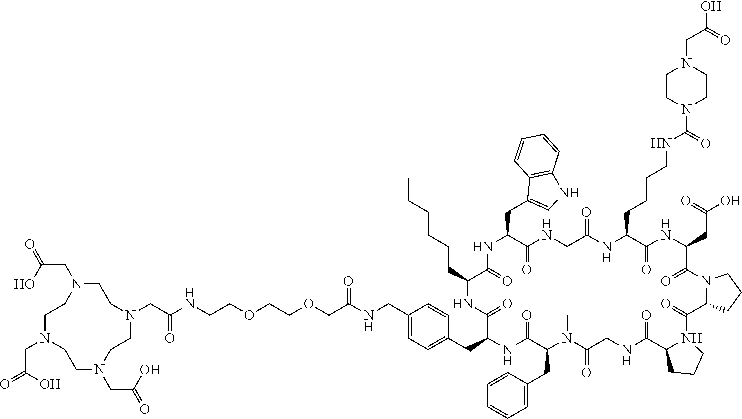 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.70 min [M + H]⁺ = 1918.0 |
| | 2,2′,2″-(10-(1-(4-(((6S,9S,15S,18S,21S,24S,29aS,34aR)-15-((1H-indol-3-yl)methyl)- | |
| | 24-benzyl-6-(carboxymethyl)-9-(4-(4-(carboxymethyl)piperazine-1- | |
| | carboxamido)butyl)-18-hexyl-25-methyl-5,8,11,14,17,20,23,26,29,34- | |
| | decaoxodotriacontahydro-1H,5H-dipyrrolo[1,2-a:1′,2′- | |
| | b₁][1,4,7,10,13,16,19,22,25,28]decaazacyclotriacontin-21-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B14 | 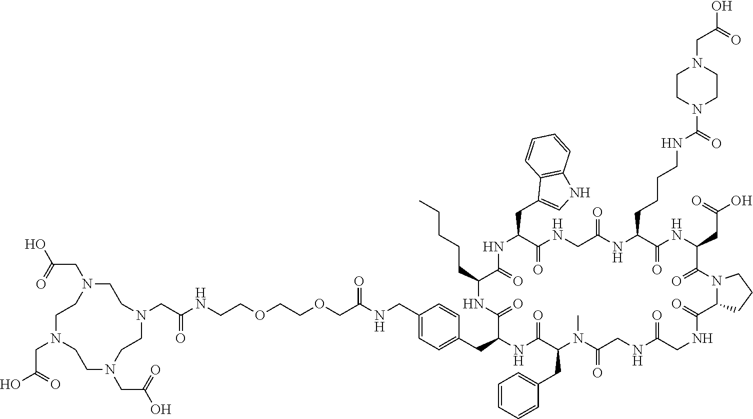 [SMILES: CCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.40 min [M + H]⁺ = 1863.9 |
| | 2,2′,2″-(10-(1-(4-(((9S,12S,15S,18S,24S,27S,32aR)-18-((1H-indol-3-yl)methyl)-9- | |
| | benzyl-27-(carboxymethyl)-24-(4-(4-(carboxymethyl)piperazine-1- | |
| | carboxamido)butyl)-8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B15 | 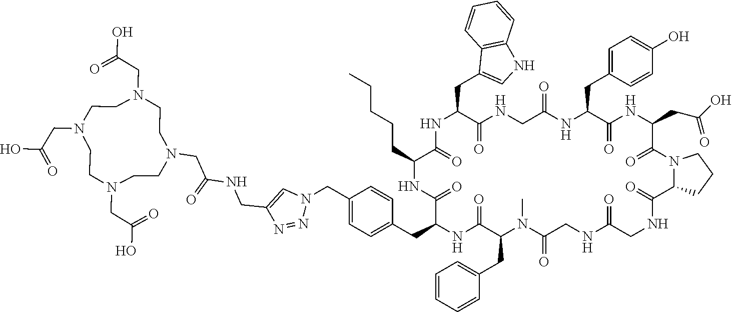 [SMILES: CCCCC[C@@H]1NC(=O)[C@H](Cc2ccc(Cn3cc(CNC(=O)CN4CCN(CC(=O)O)CCN(CC(=O)O)CCN(CC(=O)O)CC4)nn3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.85 min [M + H]⁺ = 1664.8 |
| | 2,2′,2″-(10-(2-(((1-(4-(((9S,12S,15S,18S,24S,27S,32aR)-18-((1H-indol-3-yl)methyl)- | |
| | 9-benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12-yl)methyl)benzyl)-1H- | |
| | 1,2,3-triazol-4-yl)methyl)amino)-2-oxoethyl)-1,4,7,10-tetraazacyclododecane-1,4,7- | |
| | triyl)triacetic acid TFA salt | |
| B16 | 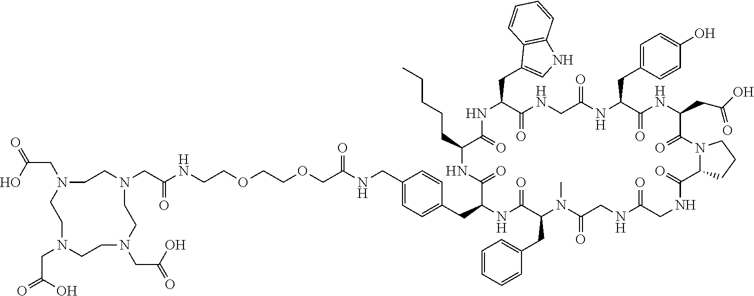 [SMILES: CCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.70 min [M + H]⁺ = 1728.8 |
| | 2,2′,2″-(10-(1-(4-(((9S,12S,15S,18S,24S,27S,32aR)-18-((1H-indol-3-yl)methyl)-9- | |
| | benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B17 | 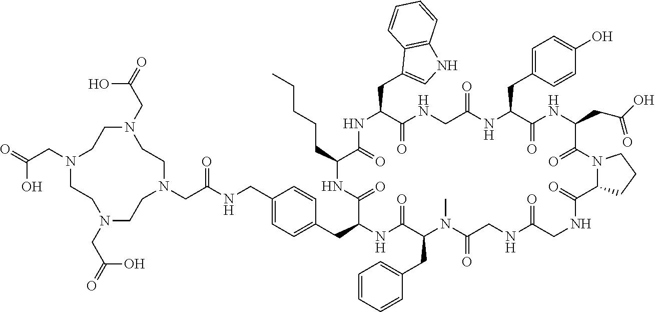 [SMILES: CCCCC[C@@H]1NC(=O)[C@H](Cc2ccc(CNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.66 min [M + H]⁺ = 1583.8 |
| | 2,2′,2″-(10-(2-((4-(((9S,12S,15S,18S,24S,27S,32aR)-18-((1H-indol-3-yl)methyl)-9- | |
| | benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12-yl)methyl)benzyl)amino)- | |
| | 2-oxoethyl)-1,4,7,10-tetraazacyclododecane-1,4,7-triyl)triacetic acid TFA salt | |
| B18 | 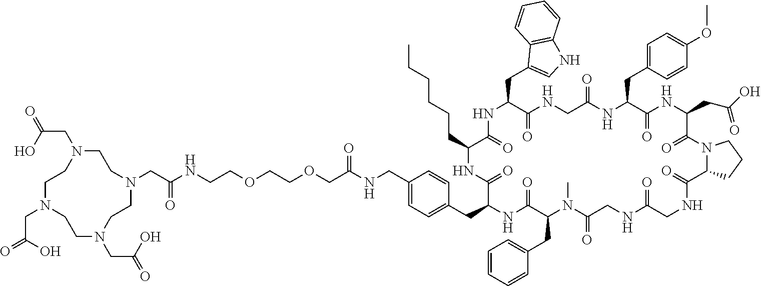 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(OC)cc2)NC(=O)CNC(=O)[C@H](Cc2c[nH]c3ccccc23)NC1=O] | Method P-1 t_{R} = 4.19 min [M + H]⁺ = 1756.9 |
| | 2,2′,2″-(10-(1-(4-(((9S,12S,15S,18S,24S,27S,32aR)-18-((1H-indol-3-yl)methyl)-9- | |
| | benzyl-27-(carboxymethyl)-15-hexyl-24-(4-methoxybenzyl)-8-methyl- | |
| | 1,4,7,10,13,16,19,22,25,28-decaoxodotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid | |
| B19 | 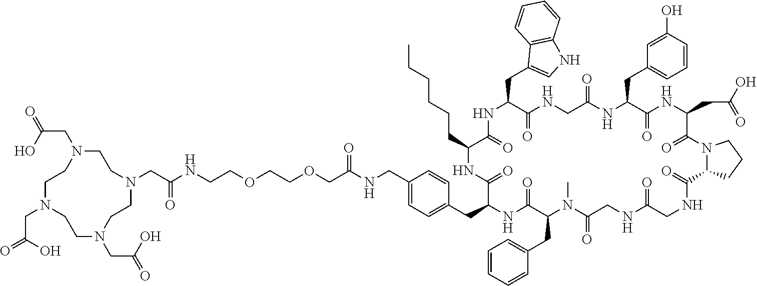 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2cccc(O)c2)NC(=O)CNC(=O)[C@H](Cc2c[nH]c3ccccc23)NC1=O] | Method P-1 t_{R} = 4.11 min [M + H]⁺ = 1742.9 |
| | 2,2′,2″-(10-(1-(4-(((9S,12S,15S,18S,24S,27S,32aR)-18-((1H-indol-3-yl)methyl)-9- | |
| | benzyl-27-(carboxymethyl)-15-hexyl-24-(3-hydroxybenzyl)-8-methyl- | |
| | 1,4,7,10,13,16,19,22,25,28-decaoxodotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B20 | 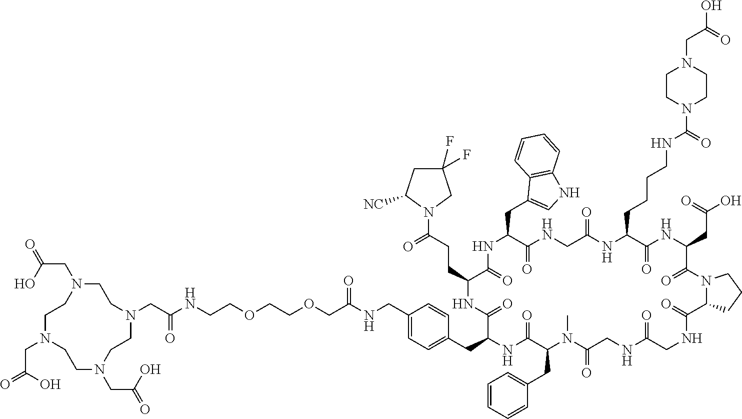 [SMILES: CN1C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC(=O)[C@H](CCC(=O)N2CC(F)(F)C[C@H]2C#N)NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@@H]1Cc1ccccc1] | Method P-1 t_{R} = 2.79 min [M + 2H]²⁺ = 990.5 |
| | 2,2′,2″-(10-(1-(4-(((9S,12S,15S,18S,24S,27S,32aR)-18-((1H-indol-3-yl)methyl)-9- | |
| | benzyl-27-(carboxymethyl)-24-(4-(4-(carboxymethyl)piperazine-1- | |
| | carboxamido)butyl)-15-(3-((S)-2-cyano-4,4-difluoropyrrolidin-1-yl)-3-oxopropyl)-8- | |
| | methyl-1,4,7,10,13,16,19,22,25,28-decaoxodotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B21 | 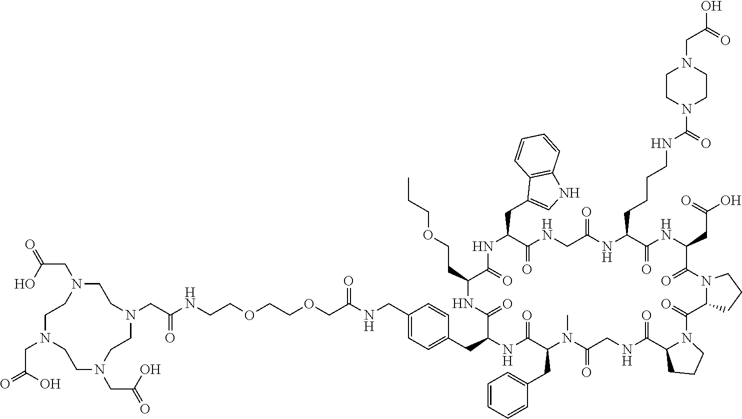 [SMILES: CCCOCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)[C@@H]2CCCN2C(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] | Method P-1 t_{R} = 3.09 min [M + 2H]²⁺ = 960.5 |
| | 2,2′,2″-(10-(1-(4-(((6S,9S,15S,18S,21S,24S,29aS,34aR)-15-((1H-indol-3-yl)methyl)- | |
| | 24-benzyl-6-(carboxymethyl)-9-(4-(4-(carboxymethyl)piperazine-1- | |
| | carboxamido)butyl)-25-methyl-5,8,11,14,17,20,23,26,29,34-decaoxo-18-(2- | |
| | propoxyethyl)dotriacontahydro-1H,5H-dipyrrolo[1,2-a:1′,2′- | |
| | b₁][1,4,7,10,13,16,19,22,25,28]decaazacyclotriacontin-21-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B22 | 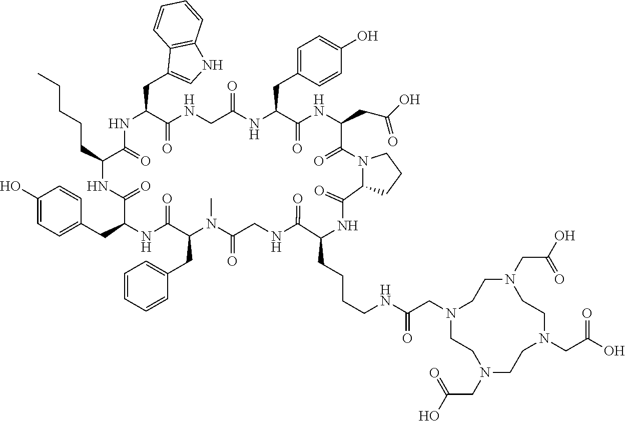 [SMILES: CCCCC[C@@H]1NC(=O)[C@H](Cc2ccc(O)cc2)NC(=O)[C@H](Cc2ccccc2)N(C)C(=O)CNC(=O)[C@H](CCCCNC(=O)CN2CCN(CC(=O)O)CCN(CC(=O)O)CCN(CC(=O)O)CC2)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.71 min [M + H]⁺ = 1641.8 |
| | 2,2′,2″-(10-(2-((4-((3S,9S,12S,15S,18S,24S,27S,32aR)-18-((1H-indol-3-yl)methyl)-9- | |
| | benzyl-27-(carboxymethyl)-12,24-bis(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-3-yl)butyl)amino)-2- | |
| | oxoethyl)-1,4,7,10-tetraazacyclododecane-1,4,7-triyl)triacetic acid TFA salt | |
| B23 | 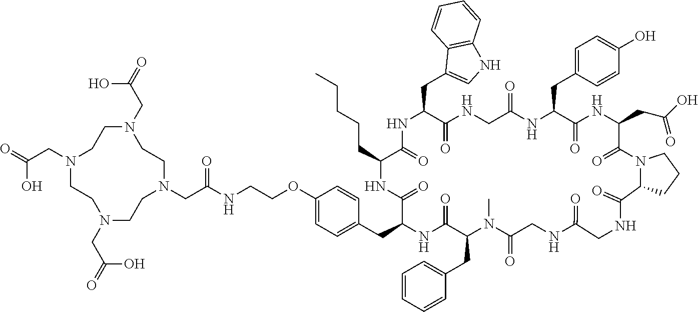 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.55 min [M + H]⁺ = 1613.8 |
| | 2,2′,2″-(10-(2-((2-(4-(((9S,12S,15S,18S,24S,27S,32aR)-18-((1H-indol-3-yl)methyl)- | |
| | 9-benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12- | |
| | yl)methyl)phenoxy)ethyl)amino)-2-oxoethyl)-1,4,7,10-tetraazacyclododecane-1,4,7- | |
| | triyl)triacetic acid TFA salt | |
| B24 | 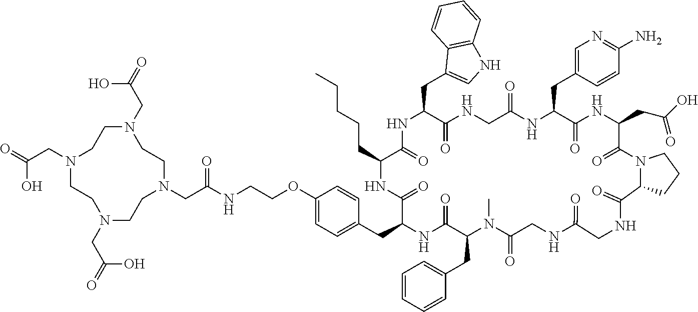 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(N)nc2)NC(=O)CNC(=O)[C@H](Cc2c[nH]c3ccccc23)NC1=O] | Method P-1 t_{R} = 3.20 min [M + H]⁺ = 1613.8 |
| | 2,2′,2″-(10-(2-((2-(4-(((9S,12S,15S,18S,24S,27S,32aR)-18-((1H-indol-3-yl)methyl)- | |
| | 24-((6-aminopyridin-3-yl)methyl)-9-benzyl-27-(carboxymethyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12- | |
| | yl)methyl)phenoxy)ethyl)amino)-2-oxoethyl)-1,4,7,10-tetraazacyclododecane-1,4,7- | |
| | triyl)triacetic acid TFA salt | |
| B25 | 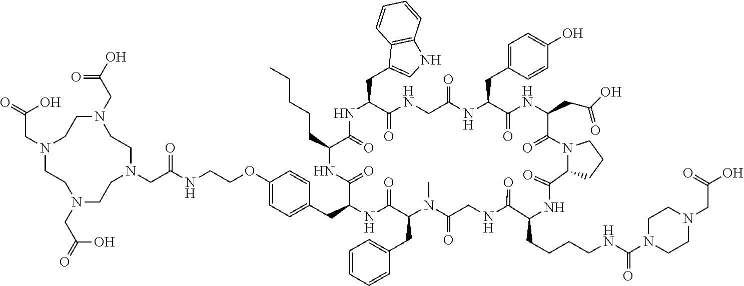 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)[C@H](CCCCNC(=O)N2CCN(CC(=O)O)CC2)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.45 min [M + H]⁺ = 1854.9 |
| | 2,2′,2″-(10-(2-((2-(4-(((3S,9S,12S,15S,18S,24S,27S,32aR)-18-((1H-indol-3- | |
| | yl)methyl)-9-benzyl-27-(carboxymethyl)-3-(4-(4-(carboxymethyl)piperazine-1- | |
| | carboxamido)butyl)-24-(4-hydroxybenzyl)-8-methyl-1,4,7,10,13,16,19,22,25,28- | |
| | decaoxo-15-pentyldotriacontahydropyrrolo[1,2- | |
| | a][1,4,7,10,13,16,19,22,25,28]decaazacyclotriacontin-12- | |
| | yl)methyl)phenoxy)ethyl)amino)-2-oxoethyl)-1,4,7,10-tetraazacyclododecane-1,4,7- | |
| | triyl)triacetic acid TFA salt | |
| B26 | 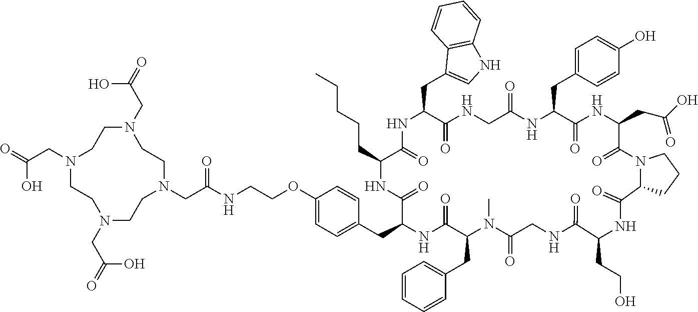 [SMILES: CCCCC[C@@H]1NC(=O)[C@H](Cc2ccc(OCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)[C@H](CCO)NC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.68 min [M + H]⁺ = 1657.8 |
| | 2,2′,2″-(10-(2-((2-(4-(((3S,9S,12S,15S,18S,24S,27S,32aR)-18-((1H-indol-3- | |
| | yl)methyl)-9-benzyl-27-(carboxymethyl)-24-(4-hydroxybenzyl)-3-(2-hydroxyethyl)- | |
| | 8-methyl-1,4,7,10,13,16,19,22,25,28-decaoxo-15- | |
| | pentyldotriacontahydropyrrolo[1,2- | |
| | a][1,4,7,10,13,16,19,22,25,28]decaazacyclotriacontin-12- | |
| | yl)methyl)phenoxy)ethyl)amino)-2-oxoethyl)-1,4,7,10-tetraazacyclododecane-1,4,7- | |
| | triyl)triacetic acid TFA salt | |
| B27 | 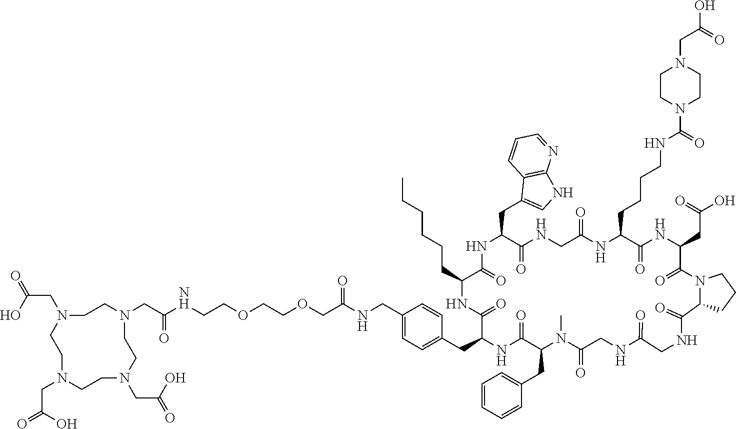 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-1 t_{R} = 2.74 min [M + H]⁺ = 1878.9 |
| | 2,2′,2″-(10-(1-(4-(((9S,12S,15S,18S,24S,27S,32aR)-18-((1H-pyrrolo[2,3-b]pyridin-3- | |
| | yl)methyl)-9-benzyl-27-(carboxymethyl)-24-(4-(4-(carboxymethyl)piperazine-1- | |
| | carboxamido)butyl)-15-hexyl-8-methyl-1,4,7,10,13,16,19,22,25,28- | |
| | decaoxodotriacontahydropyrrolo[1,2- | |
| | a][1,4,7,10,13,16,19,22,25,28]decaazacyclotriacontin-12-yl)methyl)phenyl)-3,12- | |
| | dioxo-5,8-dioxa-2,11-diazatridecan-13-yl)-1,4,7,10-tetraazacyclododecane-1,4,7- | |
| | triyl)triacetic acid TFA salt | |
| B28 | 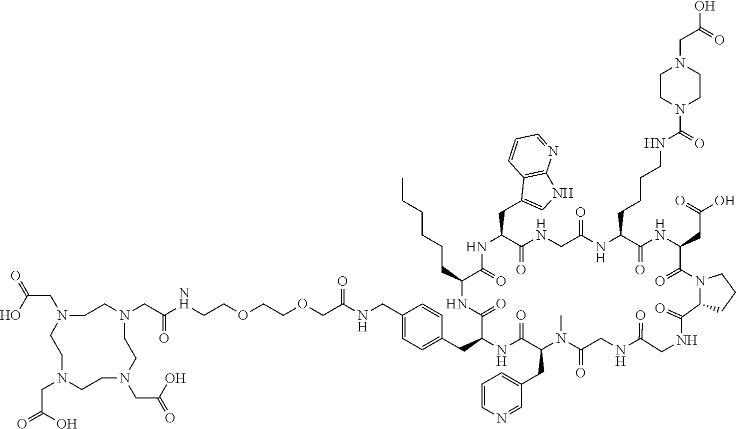 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2cccnc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ncccc23)NC1=O] | Method P-1 t_{R} = 2.74 min [M + H]⁺ = 1878.9 |

2,2′,2″-(10-(1-(4-(((9S,12S,15S,18S,24S,27S,32aR)-18-((1H-indol-3-yl)methyl)-27-
(carboxymethyl)-24-(4-(4-(carboxymethyl)piperazine-1-carboxamido)butyl)-15-
hexyl-8-methyl-1,4,7,10,13,16,19,22,25,28-decaoxo-9-(pyridin-3-
ylmethyl)dotriacontahydropyrrolo[1,2-
a][1,4,7,10,13,16,19,22,25,28]decaazacyclotriacontin-12-yl)methyl)phenyl)-3,12-
dioxo-5,8-dioxa-2,11-diazatridecan-13-yl)-1,4,7,10-tetraazacyclododecane-1,4,7-
triyl)triacetic acid TFA salt

### 3.1.2 Synthesis of 2,2′,2″-(10-(2-((2-((4-(((9S,12S,15S,18S,24S,27S,32aR)-18-((1H-indol-3-yl)methyl)-9-benzyl-27-(carboxymethyl)-24-(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-12-yl)methyl)benzyl)amino)-2-oxoethyl)(methyl)amino)-2-oxoethyl)-1,4,7,10-tetraazacyclododecane-1,4,7-triyl)triacetic acid (Example B9)

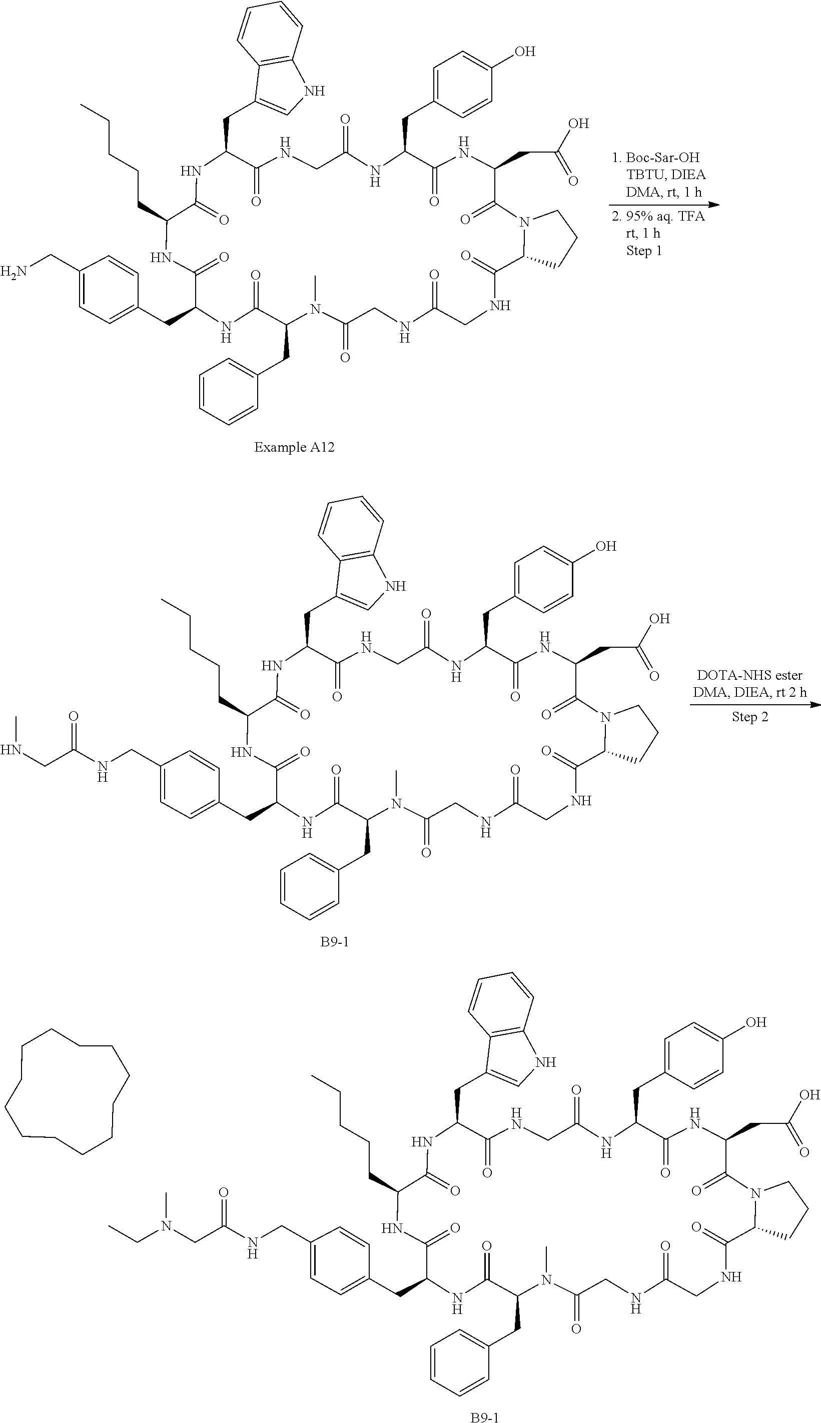

### Scheme 3.1.2. Synthesis of Example B9

### Step 1. B9-1

Step 1-1: A solution of Boc-Sar-OH (7.46 mg, 1.1 eq., 39.4 μmol), TBTU (12.7 mg, 1.1 eq., 39.4 μmol) and DIEA (5.79 mg, 7.8 μL, 1.25 eq., 44.8 μmol) in DMA (0.5 mL) was added at rt to a solution of Example A12 (47 mg, 35.8 μmol, TFA salt) and DIEA (5.79 mg, 7.80 μL, 1.25 eq., 44.8 mol) in DMA (2 mL). The reaction was stirred for 1 h at rt and then concentrated in vacuo.

Step 1-2: The residue from Step 1-1 was dissolved in 95% aq. TFA (5 mL). The solution was stirred for 1 h at rt, then concentrated to dryness in vacuo to afford B9-1.

### Step 2. Example B9

B9-1 (calculated with 35.8 μmol) and DOTA-NHS ester (41.2 mg, 1.51 eq., 54.2 μmol, trifluoroacetate-hexafluorophosphate) was dissolved in DMA (2 mL). DIEA (46.3 mg, 62.4 μL, 10 eq., 358 μmol) was added and the reaction mixture was stirred for 1 h 15 min at rt. Further DOTA-NHS ester (8.98 mg, 0.329 eq., 11.8 mol, trifluoroacetate-hexafluorophosphate) was added and the reaction mixture was stirred for 45 min at rt. The product was isolated by preparative HPLC (Column: XBridge BEH C18 OBD Prep Column, 130 Å, 5 μm, 30 mm×250 mm; Eluent A: H₂O+0.08% NH₄HCO₃ and eluent B: ACN). Pure fractions were combined and lyophilized to afford Example B9 (27.8 mg, 16 μmol, 46% yield) as a white solid. LCMS method P-1, t_{R}=3.67 min, [M+H]⁺=1654.8.

The examples in Table 3.2 were synthesized in analogy to Example B9.

Example B10 was synthesized from Example A12 using Boc-Gly-OH instead of Boc-Sar-OH.

Example B11 was synthesized from Example A12 using Boc-βAla-OH instead of Boc-Sar-OH. The DOTA-coupling was done at rt for 0.5 h, then at 50° C. for 1 h.

Example B12 was synthesized from Example A12 using Boc-NMe-βAla-OH instead of Boc-Sar-OH. The DOTA-coupling was done at 50° C. for 4 h.

TABLE 3.2
Examples including postmodification and DOTA coupling (one-pot)

| Ex- | | |
|---|---|---|
| ample | | |
| No. | Structure/Example Name | LCMS |
| B10 | 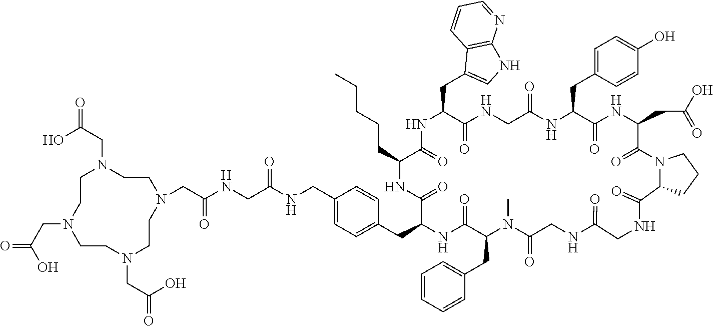 [SMILES: CCCCC[C@@H]1NC(=O)[C@H](Cc2ccc(CNC(=O)CNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ncccc23)NC1=O] | Method P-1 t_{R} = 3.68 min [M + H]⁺ = 1640.8 |
| | 2,2′,2″-(10-(2-((2-((4-(((9S,12S,15S,18S,24S,27S,32aR)-18-((1H-indol-3-yl)methyl)- | |
| | 9-benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12-yl)methyl)benzyl)amino)- | |
| | 2-oxoethyl)amino)-2-oxoethyl)-1,4,7,10-tetraazacyclododecane-1,4,7-triyl)triacetic | |
| | acid | |
| B11 | 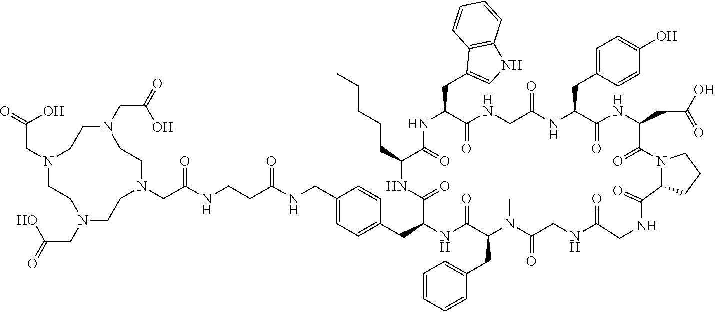 [SMILES: CCCCC[C@@H]1NC(=O)[C@H](Cc2ccc(CNC(=O)CCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.63 min [M + H]⁺ = 1654.8 |
| | 2,2′,2″-(10-(2-((3-((4-(((9S,12S,15S,18S,24S,27S,32aR)-18-((1H-indol-3-yl)methyl)- | |
| | 9-benzyl-27-(carboxymethyl)-24-(4-hydroxybenzyl)-8-methyl- | |
| | 1,4,7,10,13,16,19,22,25,28-decaoxo-15-pentyldotriacontahydropyrrolo[1,2- | |
| | a][1,4,7,10,13,16,19,22,25,28]decaazacyclotriacontin-12-yl)methyl)benzyl)amino)- | |
| | 3-oxopropyl)amino)-2-oxoethyl)-1,4,7,10-tetraazacyclododecane-1,4,7-triyl)triacetic | |
| | acid | |
| B12 | 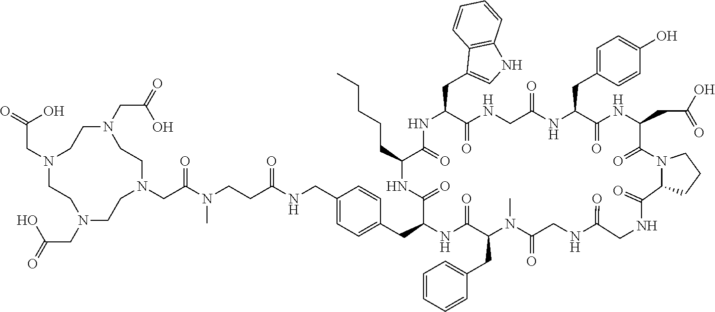 [SMILES: CCCCC[C@@H]1NC(=O)[C@H](Cc2ccc(CNC(=O)CCN(C)C(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] | Method P-1 t_{R} = 3.62 min [M + H]⁺ = 1668.8 |

2,2′,2″-(10-(2-((3-((4-(((9S,12S,15S,18S,24S,27S,32aR)-18-((1H-indol-3-yl)methyl)-
9-benzyl-27-(carboxymethyl)-24-(4-hydroxybenzyl)-8-methyl-
1,4,7,10,13,16,19,22,25,28-decaoxo-15-pentyldotriacontahydropyrrolo[1,2-
a][1,4,7,10,13,16,19,22,25,28]decaazacyclotriacontin-12-yl)methyl)benzyl)amino)-
3-oxopropyl)(methyl)amino)-2-oxoethyl)-1,4,7,10-tetraazacyclododecane-1,4,7-
triyl)triacetic acid

### 3.2 Coupling of (R)-DOTAGA

### 3.2.1 Synthesis of 2,2′,2″-(10-((R)-4-((2-(4-(((9S,12S,15S,18S,24S,27S,32aR)-18-((1H-indol-3-yl)methyl)-9-benzyl-27-(carboxymethyl)-24-(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-12-yl)methyl)phenoxy)ethyl)amino)-1-carboxy-4-oxobutyl)-1,4,7,10-tetraazacyclododecane-1,4,7-triyl)triacetic acid TFA salt (Example B30)

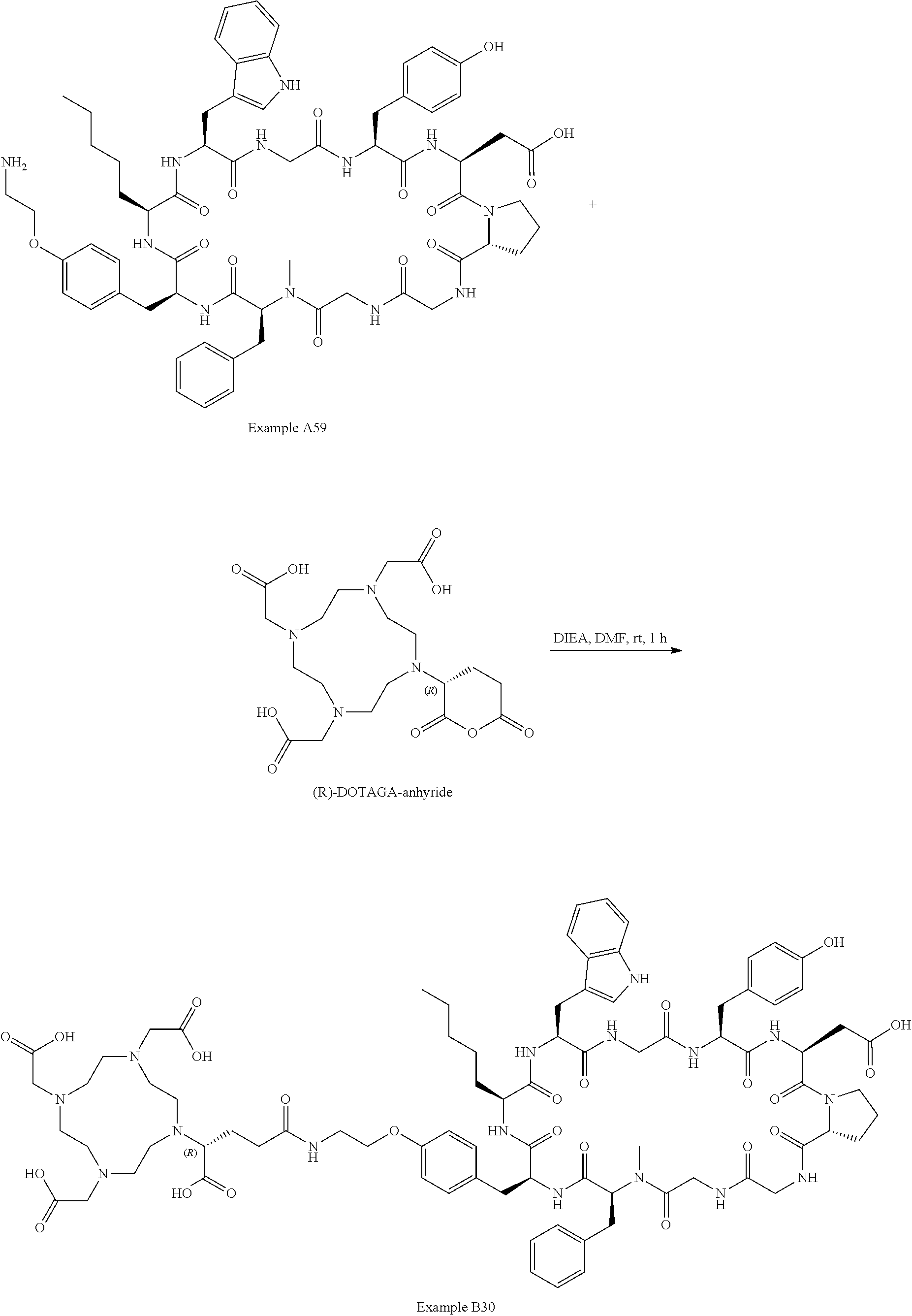

### Scheme 3.2.1. Synthesis of Example B30

To a solution of the peptide Example A59 (33.00 mg, 1 eq., 24.6 mol, TFA salt) in DMF (1.0 mL) was added (R)-DOTAGA-anhydride (purchased from CheMatech®, 25.94 mg, 2.3 eq., 56.6 mol) and DIEA (51.4 μL, 12 eq., 295.2 mol). The reaction mixture was stirred at rt for 1 h. The reaction mixture was diluted with ACN/water (1/1) (2.0 mL), filtered and the product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example B30 (21.0 mg, 11 μmol, 46% yield, TFA salt) as a white solid. LCMS method P-1, t_{R}=3.78 min, [M+H]⁺=1685.8.

The examples in Table 3.3 were synthesized in analogy to Example B30.

Example B32 was synthesized from Example A2.

Example B36 was synthesized from Example A1.

TABLE 3.3
Examples synthesized in analogy to Example B30.

| Example | | |
|---|---|---|
| No. | Structure/Example Name | LCMS |
| B32 | 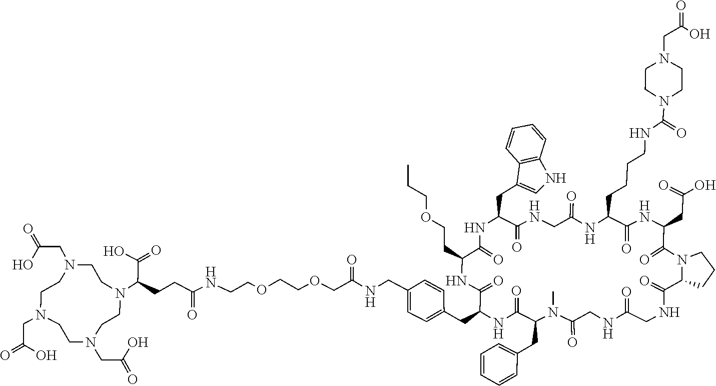 [SMILES: CCCOCC[C@@H]1NC(=O)[C@H](Cc2ccc(CNC(=O)COCCOCCNC(=O)CC[C@H](C(=O)O)N3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] 2,2′,2″-(10-((R)-1-(4-(((9S,12S,15S,18S,24S,27S,32aR)-18-((1H-indol-3-yl)methyl)-9- benzyl-27-(carboxymethyl)-24-(4-(4-(carboxymethyl)piperazine-1- carboxamido)butyl)-8-methyl-1,4,7,10,13,16,19,22,25,28-decaoxo-15-(2- propoxyethyl)dotriacontahydropyrrolo[1,2- a][1,4,7,10,13,16,19,22,25,28]decaazacyclotriacontin-12-yl)methyl)phenyl)-15- carboxy-3,12-dioxo-5,8-dioxa-2,11-diazapentadecan-15-yl)-1,4,7,10- tetraazacyclododecane-1,4,7-triyl)triacetic acid TFA salt | Method P-1 t_{R} = 3.02 min [M + H]⁺ = 1951.9 |
| B36 | 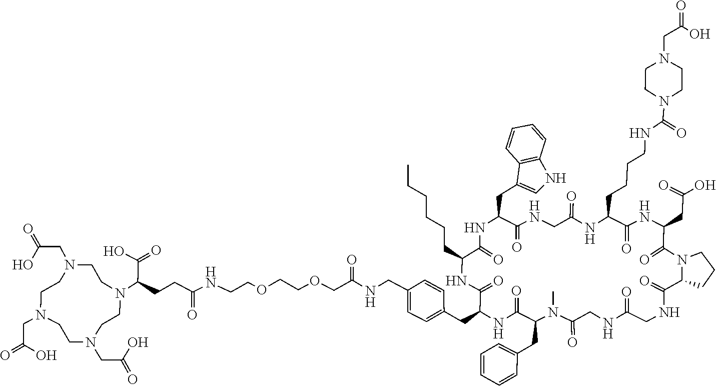 [SMILES: CCCCCC[C@@H]1NC(=O)[C@H](Cc2ccc(CNC(=O)COCCOCCNC(=O)CC[C@H](C(=O)O)N3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] 2,2′,2″-(10-((R)-1-(4-(((9S,12S,15S,18S,24S,27S,32aR)-18-((1H-indol-3-yl)methyl)-9- benzyl-27-(carboxymethyl)-24-(4-(4-(carboxymethyl)piperazine-1- carboxamido)butyl)-15-hexyl-8-methyl-1,4,7,10,13,16,19,22,25,28- decaoxodotriacontahydropyrrolo[1,2- a][1,4,7,10,13,16,19,22,25,28]decaazacyclotriacontin-12-yl)methyl)phenyl)-15- carboxy-3,12-dioxo-5,8-dioxa-2,11-diazapentadecan-15-yl)-1,4,7,10- tetraazacyclododecane-1,4,7-triyl)triacetic acid TFA salt | Method P-1 t_{R} = 3.56 min [M + H]⁺ = 1950.0 |

### 3.3 Coupling of AAZTA

### 3.3.1 Synthesis of 2,2′-((6-(1-(4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-12-yl)methyl)phenyl)-3,12-dioxo-5,8-dioxa-2,11-diazahexadecan-16-yl)-1,4-bis(carboxymethyl)-1,4-diazepan-6-yl)azanediyl)diacetic acid TFA salt (Example B39)

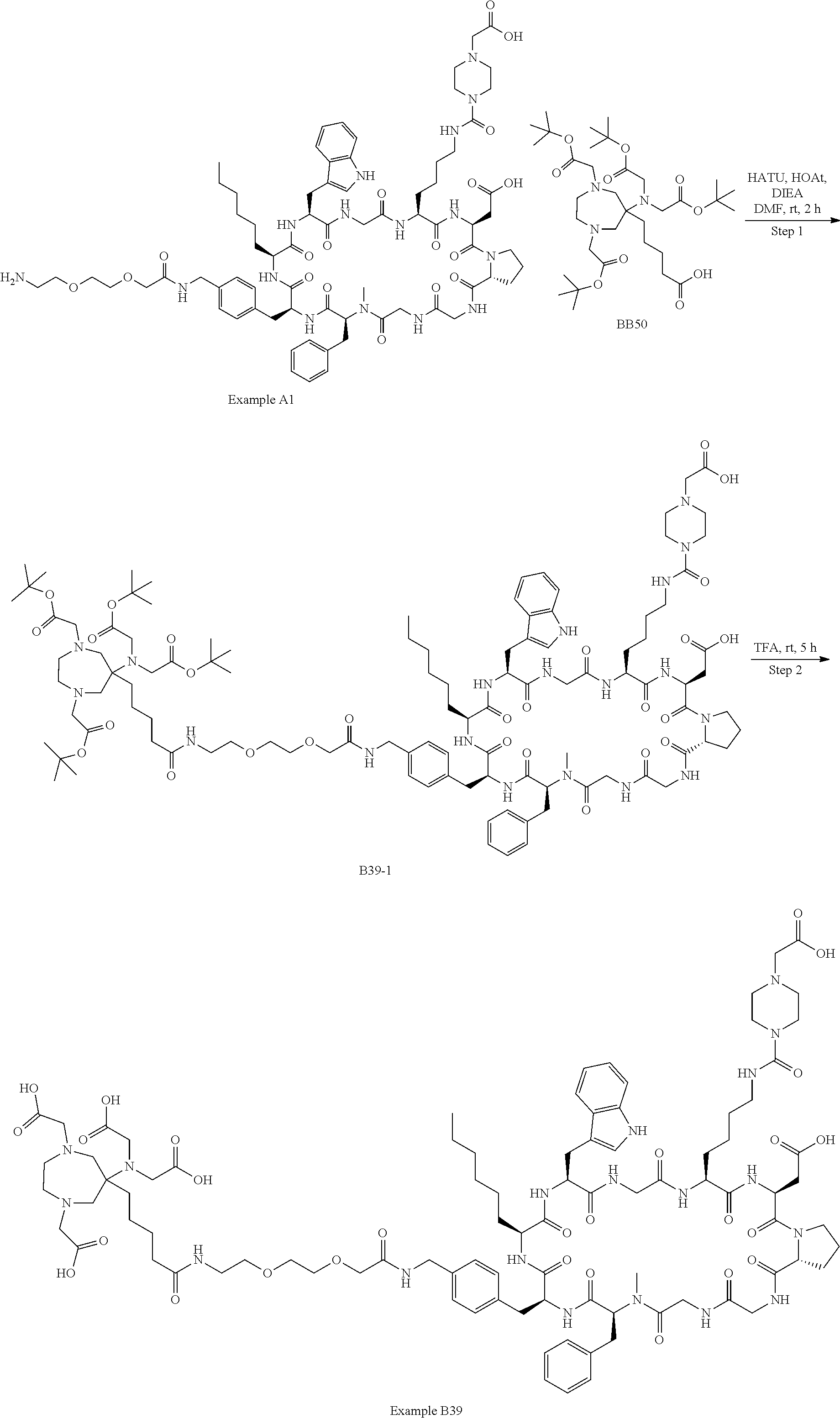

### Scheme 3.3.1. Synthesis of Example B39

### Step 1. B39-1

A solution of HATU (24.32 mg, 2.2 eq., 63.96 μmol) and HOAt (8.706 mg, 2.2 eq., 63.96 μmol) in dry DMF (750.0 L) was added to BB50 (42.97 mg, 2.2 eq., 63.96 mol). DIEA (27 μL, 5 eq.) was then added and the resulting solution was stirred for 40 min at rt. The resulting solution was added to a solution of the Example A1 (50.00 mg, 1 eq., 29.07 mol, TFA salt) in DMF (1.000 mL) and DIEA (11 μL, 2 eq.). The reaction was stirred at rt for 2 h, then concentrated under reduced pressure. The residue was dissolved in ACN/water (1/1) and purified by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford B39-1 (50.0 mg, 21 mol, 71% yield, TFA salt) as a white solid. LCMS method P-1, t_{R}=5.58 min, [M+H]⁺=2145.2.

### Step 2. Example B39

A solution of B39-1 (50.0 mg, 21 mol, TFA salt) in TFA (1 mL) was stirred at rt for 5 h. The reaction mixture was added dropwise to 30 mL of a cold solution of Et₂O/heptane (1/1) in a 50 mL Falcon tube. The resulting suspension was centrifuged for 5 min at 4700 rpm and the supernatant was removed. To the residue was added 30 mL of cold Et₂O, the suspension was shaken for 1 min, centrifuged for 5 min at 4500 rpm and the supernatant was removed. The washing step was repeated once. The precipitate was dried under high vacuum. The residue was dissolved in ACN/water (1/1) (2 mL), filtered and purified by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example B39 (15.0 mg, 6.8 μmol, 32% yield, TFA salt) as a white solid. LCMS method P-1, t_{R}=3.72 min, [M+H]⁺=1920.9.

The example in Table 3.4 was synthesized in analogy to Example B39.

Example B31 was synthesized from Example A2.

TABLE 3.4
Example synthesized in analogy to Example B39

| Example | | |
|---|---|---|
| No. | Structure/Example Name | LCMS |
| B31 | 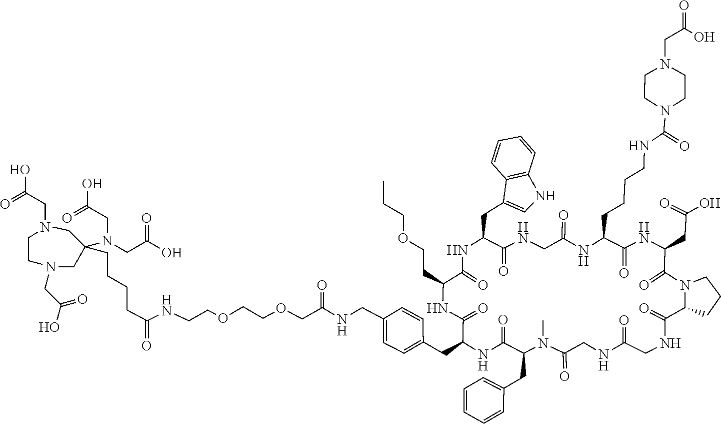 [SMILES: CCCOCC[C@@H]1NC(=O)[C@H](Cc2ccc(CNC(=O)COCCOCCNC(=O)CCCCC3(N(CC(=O)O)CC(=O)O)CN(CC(=O)O)CCN(CC(=O)O)C3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] 2,2′-((6-(1-(4-(((9S,12S,15S,18S,24S,27S,32aR)-18-((1H-indol-3-yl)methyl)-9- benzyl-27-(carboxymethyl)-24-(4-(4-(carboxymethyl)piperazine-1- carboxamido)butyl)-8-methyl-1,4,7,10,13,16,19,22,25,28-decaoxo-15-(2- propoxyethyl)dotriacontahydropyrrolo[1,2- a][1,4,7,10,13,16,19,22,25,28]decaazacyclotriacontin-12-yl)methyl)phenyl)-3,12- dioxo-5,8-dioxa-2,11-diazahexadecan-16-yl)-1,4-bis(carboxymethyl)-1,4-diazepan- 6-yl)azanediyl)diacetic acid TFA salt | Method P-1 t_{R} = 3.28 min [M + H]⁺ = 1922.9 |

### 3.4 Coupling of NODAGA

### 3.4.1 Synthesis of 2,2′-(7-(1-(4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-8-methyl-1,4,7,10,13,16,19,22,25,28-decaoxo-15-(2-propoxyethyl)dotriacontahydropyrrolo[1,2-a][1,4,7,10,13,16,19,22,25,28]decaazacyclotriacontin-12-yl)methyl)phenyl)-15-carboxy-3,12-dioxo-5,8-dioxa-2,11-diazapentadecan-15-yl)-1,4,7-triazonane-1,4-diyl)diacetic acid TFA salt (Example B33)

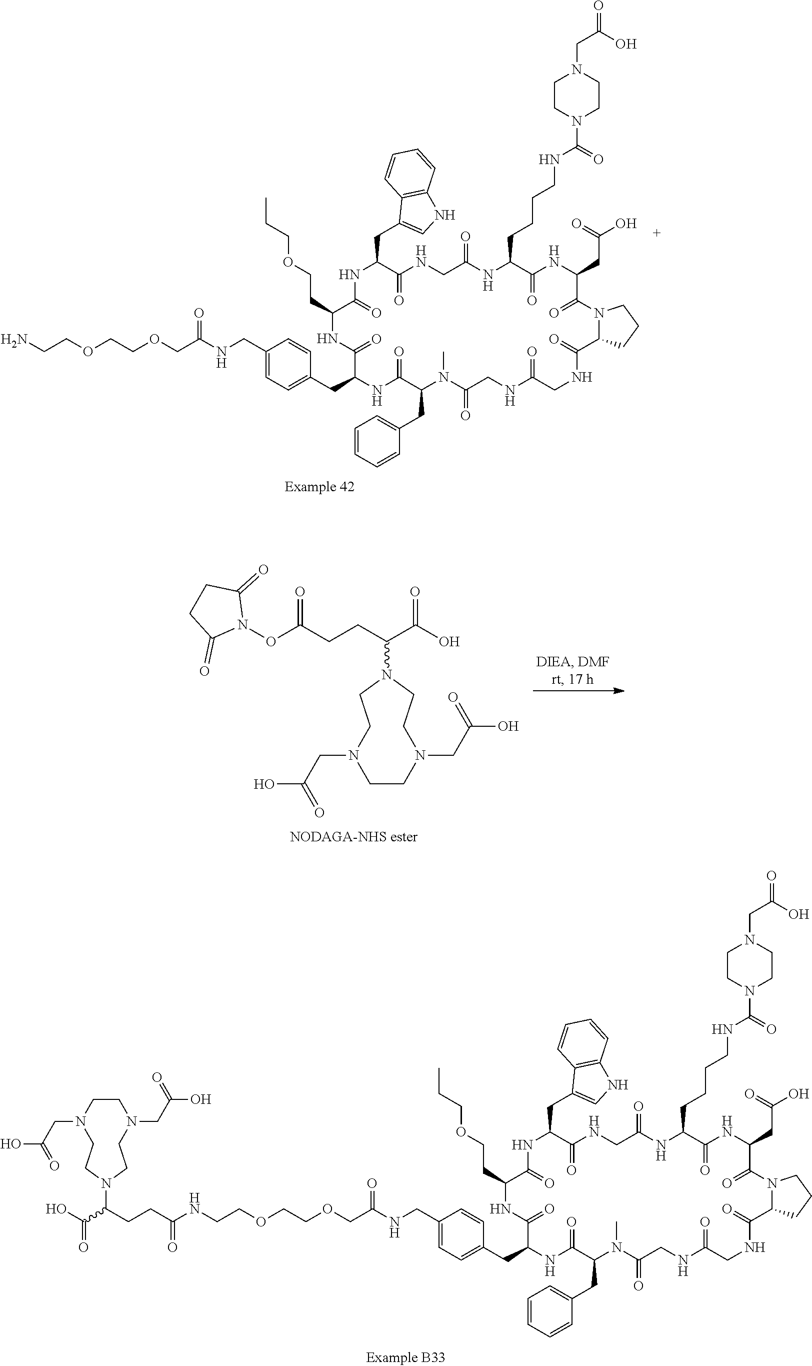

### Scheme 3.4.1. Synthesis of Example B33

To a solution of Example A2 (53.00 mg, 1 eq., 30.78 mol, TFA salt) in DMF (750.0 L) was added NODAGA-NHS ester (CAS 1407166-70-4, 16.00 mg, 1.1 eq., 33.86 mol, trifluoroacetate-hexafluorophosphate salt) and DIPEA (23.87 mg, 32.2 μL, 6 eq., 184.7 mol). The reaction mixture was stirred for at 16 h. Another portion of NODAGA-NHS ester (CAS 1407166-70-4, 10.00 mg, 0.69 eq., 21.16 mol, trifluoroacetate-hexafluorophosphate salt) and DIPEA (23.87 mg, 32.2 μL, 6 eq., 184.7 mol) were added and the reaction mixture was stirred at rt for 1 h. The reaction mixture was diluted in ACN/water 1/1 (2 mL), filtered and the product was isolated by preparative HPLC (Column: XSelect Peptide CSH C18 OBD Prep Column, 130 Å, 5 m, 30 mm×250 mm; 30 mL/min; Eluent A: H₂O+0.1% TFA and eluent B: ACN). Pure fractions were combined and lyophilized to afford Example B33 (43.0 mg, 20 μmol, 66% yield, TFA salt) as a white solid. LCMS method P-1, t_{R}=3.25 min [M+H]⁺=1850.9.

The examples in Table 3.5 were synthesized in analogy to Example B33.

Example B29 was synthesized from Example A59.

Example B38 was synthesized from Example A1.

TABLE 3.5
Examples synthesized in analogy to Example B33

| Example | | |
|---|---|---|
| No. | Structure/Example Name | LCMS |
| B29 | 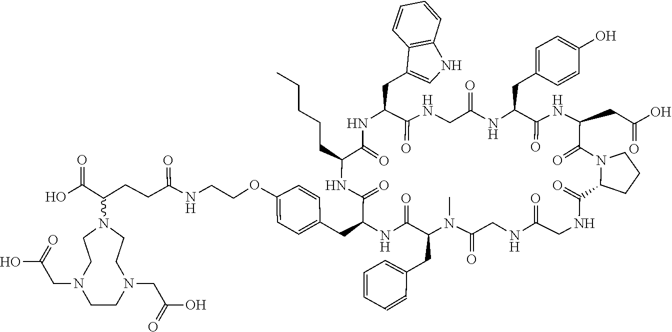 [SMILES: CCCCC[C@@H]1NC(=O)[C@H](Cc2ccc(OCCNC(=O)CCC(C(=O)O)N3CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](Cc2ccc(O)cc2)NC(=O)CNC(=O)[C@H](Cc2c[nH]c3ccccc23)NC1=O] 2,2′-(7-(4-((2-(4-(((9S,12S,15S,18S,24S,27S,32aR)-18-((1H-indol-3-yl)methyl)-9- benzyl-27-(carboxymethyl)-24-(4-hydroxybenzyl)-8-methyl- 1,4,7,10,13,16,19,22,25,28-decaoxo-15-pentyldotriacontahydropyrrolo[1,2- a][1,4,7,10,13,16,19,22,25,28]decaazacyclotriacontin-12- yl)methyl)phenoxy)ethyl)amino)-1-carboxy-4-oxobutyl)-1,4,7-triazonane-1,4- diyl)diacetic acid | Method P-1 t_{R} = 4.00 min [M + H]⁺ = 1584.7 |
| B38 | 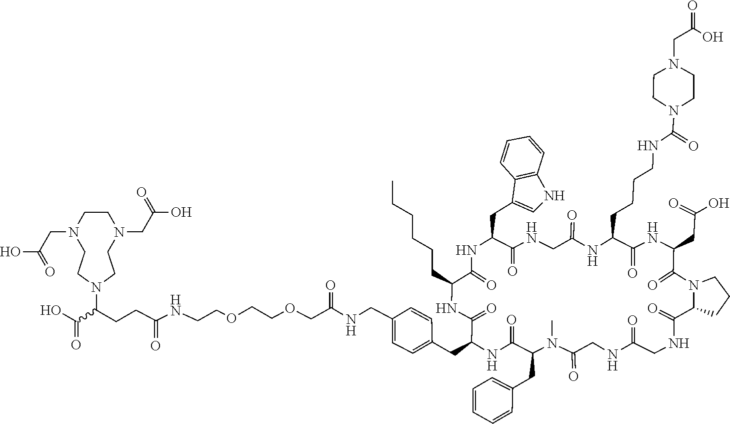 [SMILES: CCCCCC[C@@H]1NC(=O)[C@H](Cc2ccc(CNC(=O)COCCOCCNC(=O)CCC(C(=O)O)N3CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] 2,2′-(7-(1-(4-(((9S,12S,15S,18S,24S,27S,32aR)-18-((1H-indol-3-yl)methyl)-9-benzyl- 27-(carboxymethyl)-24-(4-(4-(carboxymethyl)piperazine-1-carboxamido)butyl)-15- hexyl-8-methyl-1,4,7,10,13,16,19,22,25,28-decaoxodotriacontahydropyrrolo[1,2- a][1,4,7,10,13,16,19,22,25,28]decaazacyclotriacontin-12-yl)methyl)phenyl)-15- carboxy-3,12-dioxo-5,8-dioxa-2,11-diazapentadecan-15-yl)-1,4,7-triazonane-1,4- diyl)diacetic acid TFA salt | Method P-1 t_{R} = 3.69 min [M + H]⁺ = 1848.9 |

### 3.5 Coupling of NOTA

### 3.5.1 Synthesis of 2,2′-(7-(1-(4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-12-yl)methyl)phenyl)-3,12-dioxo-5,8-dioxa-2,11-diazatridecan-13-yl)-1,4,7-triazonane-1,4-diyl)diacetic acid TFA salt (Example B40)

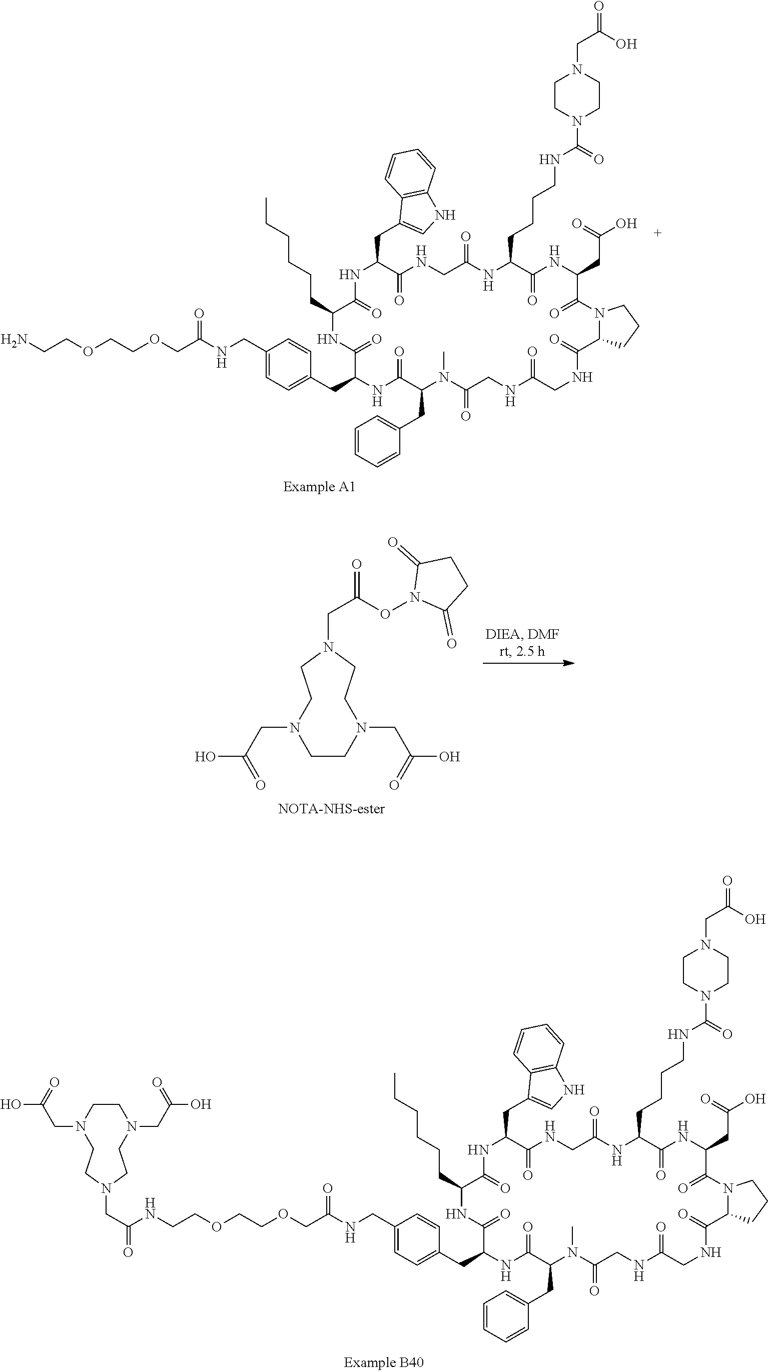

### Scheme 3.5.1. Synthesis of Example B40

To a solution of Example A1 (58.00 mg, 1 eq., 33.73 mol, TFA salt) in DMF (1000 L) was added NOTA-NHS-Ester (CAS 1338231-09-6, 33.36 mg, 1.5 eq., 50.59 μmol, trifluoroacetate-hexafluorophosphate) and DIPEA (26.15 mg, 35.2 NL, 6 eq., 202.4 mol). The reaction mixture was stirred for at rt for 2.5 h. The product was isolated by preparative HPLC (Column: XSelect Peptide CSH C18 OBD Prep Column, 130 Å, 5 m, 30 mm×250 mm; 30 mL/min; Eluent A: H₂O+0.1% TFA and eluent B: ACN). Pure fractions were combined and lyophilized to afford Example B40 (53.0 mg, 25 μmol, 75% yield, TFA salt) as a white solid. LCMS method P-M, t_{R}=3.64 m1 [M+H]⁺=1776.9.

The example in Table 3.6 was synthesized in analogy to Example B40.

Example B34 was synthesized from Example A2.

TABLE 3.6
Example B34 was synthesized in analogy to Example B40

| Example | | |
|---|---|---|
| No. | Structure/Example Name | LCMS |
| B34 | 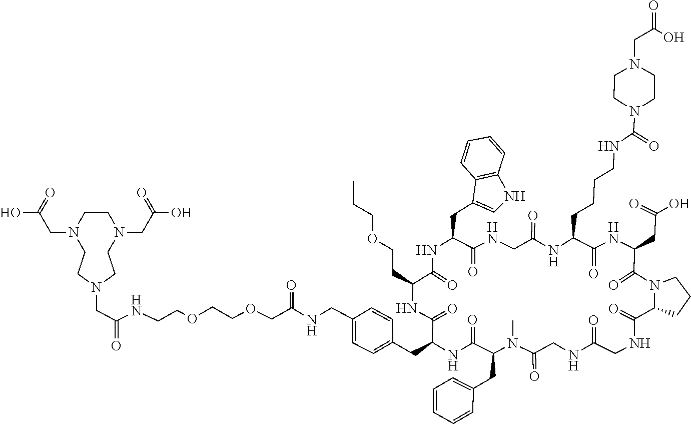 [SMILES: CCCOCC[C@@H]1NC(=O)[C@H](Cc2ccc(CNC(=O)COCCOCCNC(=O)CN3CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] 2,2′-(7-(1-(4-(((9S,12S,15S,18S,24S,27S,32aR)-18-((1H-indol-3-yl)methyl)-9-benzyl- 27-(carboxymethyl)-24-(4-(4-(carboxymethyl)piperazine-1-carboxamido)butyl)-8- methyl-1,4,7,10,13,16,19,22,25,28-decaoxo-15-(2- propoxyethyl)dotriacontahydropyrrolo[1,2- a][1,4,7,10,13,16,19,22,25,28]decaazacyclotriacontin-12-yl)methyl)phenyl)-3,12- dioxo-5,8-dioxa-2,11-diazatridecan-13-yl)-1,4,7-triazonane-1,4-diyl)diacetic acid TFA salt | Method P-1 t_{R} = 3.20 min [M + H]⁺ = 1778.9 |

### 3.6 Coupling of p-SCN-Bn-DOTA

### 3.6.1 Synthesis of 2,2′,2″,2′″-(2-(4-(3-(2-(2-(2-((4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-12-yl)methyl)benzyl)amino)-2-oxoethoxy)ethoxy)ethyl)thioureido)benzyl)-1,4,7,10-tetraazacyclododecane-1,4,7,10-tetrayl)tetraacetic acid TFA salt (Example B37)

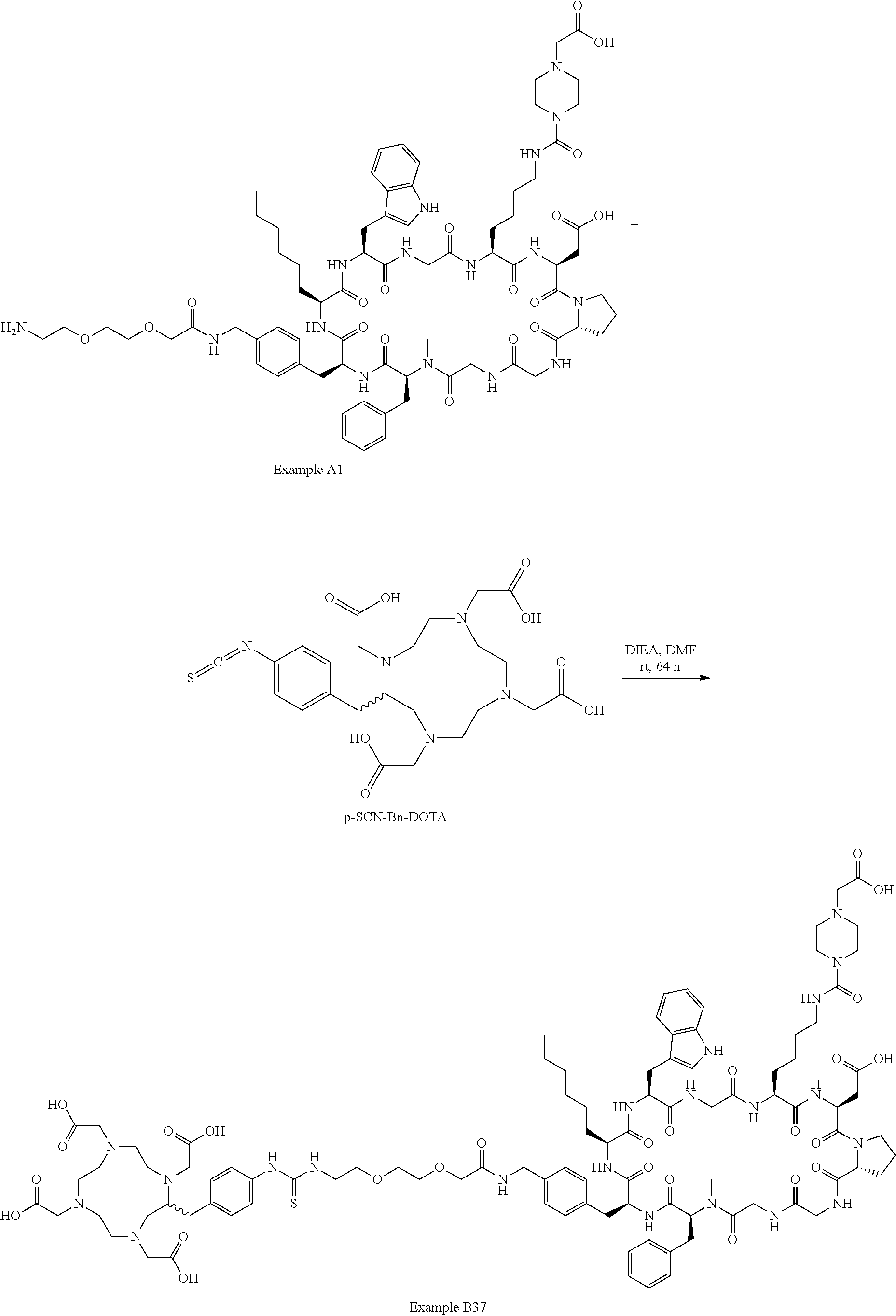

### Scheme 3.6.1. Synthesis of Example B37

To a solution of Example A1 (47.50 mg, 1 eq., 27.62 mol, TFA salt) in DMF (500.0 L) was added p-SCN-Bn-DOTA (CAS 127985-74-4, 18.28 mg, 1.2 eq., 33.14 mol) and DIPEA (14.28 mg, 19.2 μL, 4.0 eq., 110.5 μmol). The resulting solution was stirred at rt for 64 h. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example B37 (27.0 mg, 11.6 μmol, 42% yield, TFA salt) as a white solid. LCMS method P-1, t_{R}=3.71 min, [M+H]⁺=2043.0.

The examples in Table 3.7 were synthesized in analogy to Example B37.

Example B35 was synthesized from Example A2.

TABLE 3.7
Example synthesized in analogy to Example B37

| Example | | |
|---|---|---|
| No. | Structure/Example Name | LCMS |
| B35 | 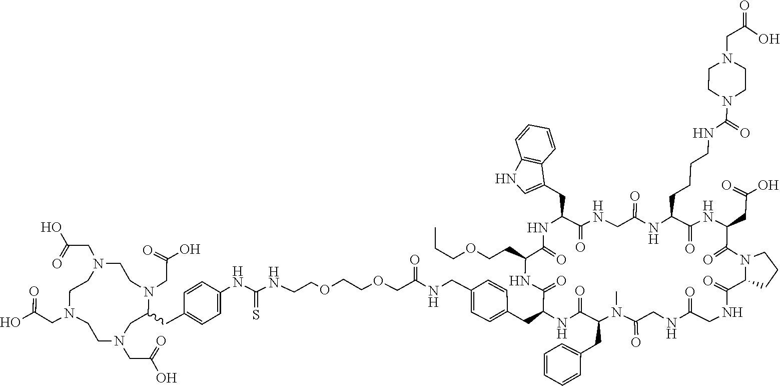 [SMILES: CCCOCC[C@@H]1NC(=O)[C@H](Cc2ccc(CNC(=O)COCCOCCNC(=S)Nc3ccc(CC4CN(CC(=O)O)CCN(CC(=O)O)CCN(CC(=O)O)CCN4CC(=O)O)cc3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O] 2,2′,2″,2′″-(2-(4-(3-(2-(2-(2-((4-(((9S,12S,15S,18S,24S,27S,32aR)-18-((1H-indol-3- yl)methyl)-9-benzyl-27-(carboxymethyl)-24-(4-(4-(carboxymethyl)piperazine-1- carboxamido)butyl)-8-methyl-1,4,7,10,13,16,19,22,25,28-decaoxo-15-(2- propoxyethyl)dotriacontahydropyrrolo[1,2- a][1,4,7,10,13,16,19,22,25,28]decaazacyclotriacontin-12-yl)methyl)benzyl)amino)-2- oxoethoxy)ethoxy)ethyl)thioureido)benzyl)-1,4,7,10-tetraazacyclododecane-1,4,7,10- tetrayl)tetraacetic acid TFA salt | Method P-1 t_{R} = 3.28 min [M + H]⁺ = 2045.0 |

### 4 Compounds Labeled with Natural Isotopes (Examples C)

### 4.1 Labelling with Natural Lutetium (Lu)

### 4.1.1 Synthesis of [2,2′,2″-(10-{1-[4-({(9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxodotriacontahydropyrrolo[1,2-a][1,4,7,10,13,16,19,22,25,28]decaazacyclotriacontin-12-yl}methyl)phenyl]-3-oxo-12-(oxo-κO)-5,8-dioxa-2,11-diazatridecan-13-yl}-1,4,7,10-tetraazacyclododecane-1,4,7-triyl-κ⁴N¹,N⁴,N⁷,N¹⁰)tri(acetato-κO)(3-)]lutetium, Example C1, (Example B1 labelled with Lu)

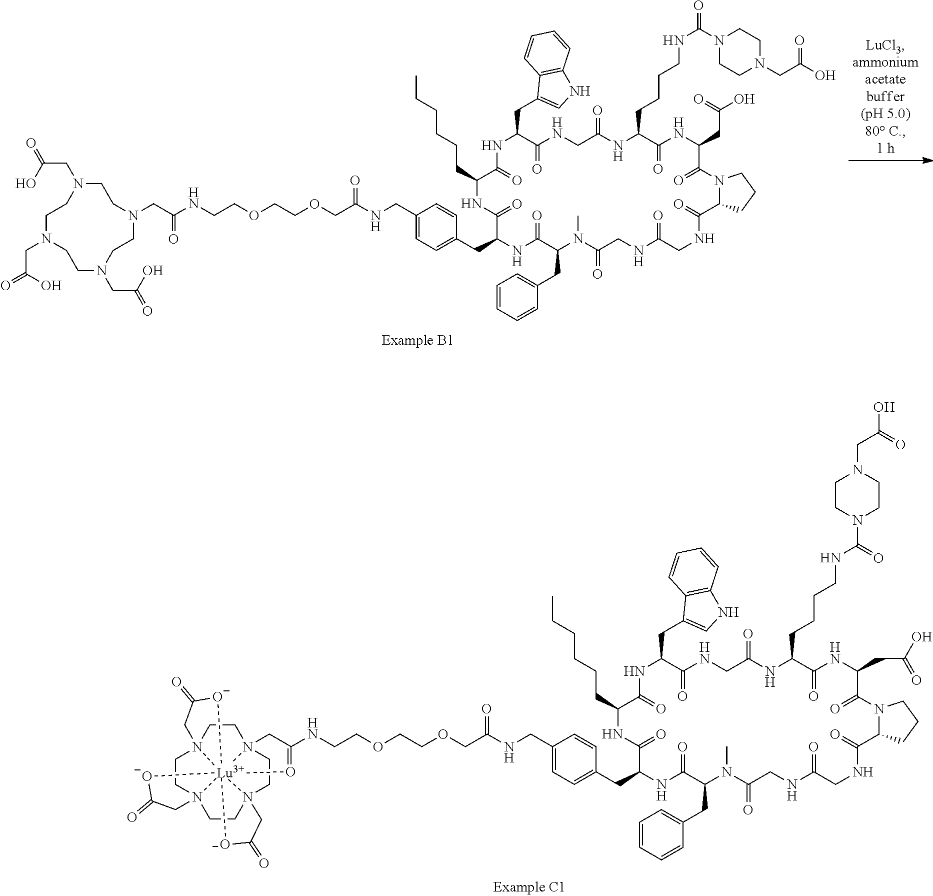

### Scheme 4.1.1. Synthesis of Example C1

To Example B1 (21.10 mg, 1 eq., 10.67 mol) in ammonium acetate buffer pH 5.0 (4.0 mL, 50 mM) was added 100 mM LuCl₃ in ammonium acetate buffer pH 5.0 (117.4 μL, 100 mM, 1.1 eq., 11.74 mol). The reaction mixture was stirred at 80° C. for 1 h. The reaction mixture was diluted with H₂O (4.0 mL) and the product was isolated by preparative HPLC (Column: XBridge BEH C18 OBD Prep Column, 130 Å, 5 μm, 30 mm×100 mm; 30 mL/min; Eluent A: H₂O+0.08% NH4HCO₃ and eluent B: ACN). Pure fractions were combined and lyophilized to afford Example C1 (17.5 mg, 8.4 mol, 78% yield) as a white solid. LCMS method P-1, t_{R}=3.66 m, [M+H]⁺=2049.9. The following examples (see Table 4.1) were synthesized in analogy to Example C1:

TABLE 4.1
Examples synthesized in analogy to Example C1

| Example | | |
|---|---|---|
| No. | Description | LCMS |
| C2 | Example B2 | Method P-2, t_{R} = 6.11 min, |
| | labelled with Lu | [M + H]⁺ = 2052.1 |
| C3 | Example B4 | Method P-1, t_{R} = 1.83 min, |
| | labelled with Lu | [M + 2H]²⁺ = 1027.4 |
| C4 | Example B5 | Method P-1, t_{R} = 3.26 min, |
| | labelled with Lu | [M + 2H]²⁺ = 1048.6 |
| C5 | Example B6 | Method P-1, t_{R} = 3.28 min, |
| | labelled with Lu | [M + 2H]²⁺ = 1062.4 |
| C6 | Example B7 | Method P-2, t_{R} = 2.80 min, |
| | labelled with Lu | [M + 2H]²⁺ = 1049.4 |
| C7 | Example B8 | Method P-1, t_{R} = 1.83 min, |
| | labelled with Lu | [M + 2H]²⁺ = 1063.6 |
| C8 | Example B9 | Method P-1, tR = 4.02 min, |
| | labelled with Lu | [M + H]⁺ = 1826.7 |
| C9 | Example B10 | Method P-1, t_{R} = 4.01 min, |
| | labelled with Lu | [M + H]⁺ = 1812.7 |
| C10 | Example B11 | Method P-1, t_{R} = 4.03 min, |
| | labelled with Lu | [M + H]⁺ = 1826.7 |
| C11 | Example B12 | Method P-1, t_{R} = 4.04 min, |
| | labelled with Lu | [M + H]⁺ = 1840.8 |
| C12 | Example B13 | Method P-1, t_{R} = 3.79 min, |
| | labelled with Lu | [M + H]⁺ = 2089.9 |
| C13 | Example B14 | Method P-1, t_{R} = 3.46 min, |
| | labelled with Lu | [M + H]⁺ = 2035.9 |
| C14 | Example B15 | Method P-1, t_{R} = 4.03 min, |
| | labelled with Lu | [M + H]⁺ = 1836.7 |
| C15 | Example B16 | Method P-1, t_{R} = 4.02 min, |
| | labelled with Lu | [M + H]⁺ = 1900.8 |
| C16 | Example B17 | Method P-1, t_{R} = 3.95 min, |
| | labelled with Lu | [M + H]⁺ = 1755.7 |
| C17 | Example B18 | Method P-1, t_{R} = 4.57 min, |
| | labelled with Lu | [M + H]⁺ = 1928.9 |
| C18 | Example B19 | Method P-1, t_{R} = 4.33 min, |
| | labelled with Lu | [M + H]⁺ = 1914.8 |
| C19 | Example B20 | Method P-1, tR = 3.01 min, |
| | labelled with Lu | [M + 2H]²⁺ = 1076.4 |
| C20 | Example B21 | Method P-1, t_{R} = 3.33 min, |
| | labelled with Lu | [M + 2H]²⁺ = 1046.4 |
| C22 | Example B22 | Method P-1, t_{R} = 4.36 min, |
| | labelled with Lu | [M + H]⁺ = 1813.7 |
| C23 | Example B23 | Method P-1, t_{R} = 4.05 min, |
| | labelled with Lu | [M + H]⁺ = 1785.7 |
| C24 | Example B24 | Method P-1, t_{R} = 3.40 min, |
| | labelled with Lu | [M + H]⁺ = 1785.7 |
| C25 | Example B25 | Method P-1, t_{R} = 3.70 min, |
| | labelled with Lu | [M + H]⁺ = 2026.9 |
| C26 | Example B26 | Method P-1, t_{R} = 4.01 min, |
| | labelled with Lu | [M + H]⁺ = 1829.7 |
| C27 | Example B3 | Method P-1, t_{R} = 3.43 min, |
| | labelled with Lu | [M + H]⁺ = 1920.8 |
| C28 | Example B27 | Method P-1, t_{R} = 2.92 min, |
| | labelled with Lu | [M + H]⁺ = 2050.9 |
| C29 | Example B28 | Method P-1, t_{R} = 2.89 min, |
| | labelled with Lu | [M + H]⁺ = 2050.9 |
| C30 | Example B29 | Method P-1, t_{R} = 4.07 min, |
| | labelled with Lu | [M + H]⁺ = 1756.7 |
| C31 | Example B30 | Method P-1, t_{R} = 4.69 min, |
| | labelled with Lu | [M + 2H]²⁺ = 929.4 |
| C32 | Example B31 | Method P-1, t_{R} = 3.57 min, |
| | labelled with Lu | [M + H]⁺ = 2094.8 |
| C33 | Example B32 | Method P-1, t_{R} = 3.50 min, |
| | labelled with Lu | [M + H]⁺ = 2123.9. |
| C34 | Example B36 | Method P-1, t_{R} = 4.05 min, |
| | labelled with Lu | [M + H]⁺ = 2121.9 |
| C35 | Example B37 | Method P-4, t_{R} = 3.05 min, |
| | labelled with Lu | [M + 2H]²⁺ = 1107.9 |
| C36 | Example B38 | Method P-1, t_{R} = 3.79 min, |
| | labelled with Lu | [M + H]⁺ = 2020.8 |
| C37 | Example B39 | Method P-1, t_{R} = 4.03 min, |
| | labelled with Lu | [M + H]⁺ = 2092.9 |

### 4.1.2 DOTA coupling and labelling with Lu (one-pot). Synthesis of (Cyclo{3-(1H-pyrrolo[2,3-b]pyridin-3-yl)-L-alanylglycyl-N⁶-[4-(carboxymethyl)piperazine-1-carbonyl]-L-lysyl-L-α-aspartyl-D-prolylglycylglycyl-N-methyl-L-phenylalanyl-4-[3-oxo-12-(oxo-κO)-13-{4,7,10-tris[(carboxy-κO)methyl]-1,4,7,10-tetraazacyclododecan-1-yl-κ⁴N¹,N⁴,N⁷,N¹⁰}-5,8-dioxa-2,11-diazatridecan-1-yl]-L-phenylalanyl-O-propyl-L-homoseryl})lutetium (Example C21)

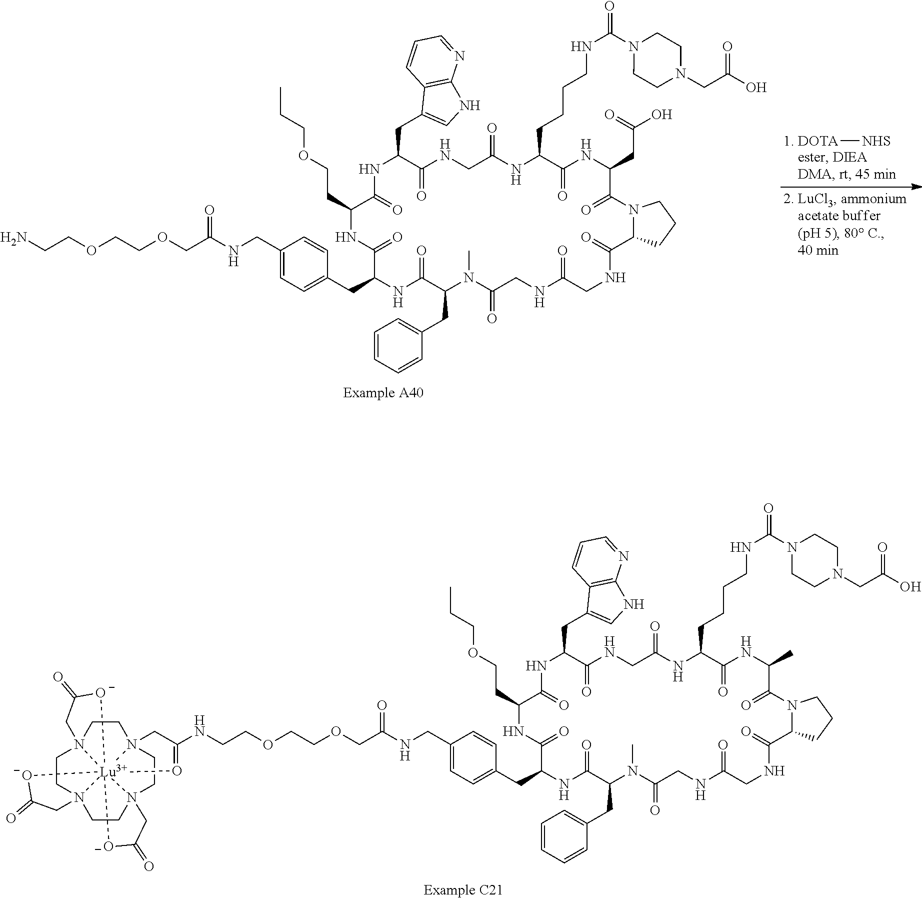

### Scheme 4.1.2. Synthesis of Example C21

### Step 1

To a clear solution of the Example A40 (11.9 mg, 6.48 mol, TFA salt) in DMA (1 mL) at rt were added DOTA-NHS ester (7.39 mg, 1.5 eq., 9.72 mol, trifluoroacetate-hexafluorophosphate), followed by DIEA (8.37 mg, 11.3 μL, 10 eq., 64.8 mol). The reaction mixture was stirred at rt for 45 min, then cooled in an ice-water bath to 0° C.

### Step 2

Then 100 mM LuCl₃ in ammonium acetate buffer pH 5.0 (1.30 mL, 100 mM, 20 eq., 130 mol) was added and the reaction was stirred at 80° C. for 40 min. The product was isolated by preparative HPLC (Column: Waters XBridge C18 OBD, 30×250 mm, 5 μm; Solvent A: Water+0.08% NH4HCO₃, Solvent B: ACN). Pure fractions were combined and lyophilized to afford Example C21 (10 mg, 4.8 μmol, 70% yield) as a white solid. LCMS method P-1, t_{R}=2.52 min, [M+H]⁺=2052.8.

### 4.2 Labelling with Natural Gallium (Ga)

### 4.2.1 Synthesis of [2,2′,2″-(10-{1-[4-({(9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxodotriacontahydropyrrolo[1,2-a][1,4,7,10,13,16,19,22,25,28]decaazacyclotriacontin-12-yl}methyl)phenyl]-3,12-dioxo-5,8-dioxa-2,11-diazatridecan-13-yl}-1,4,7,10-tetraazacyclododecane-1,4,7-triyl-κ⁴N¹,N⁴,N⁷,N¹⁰)tri(acetato-κO)(3-)]gallium Example C44 (Example B1 labelled with Ga)

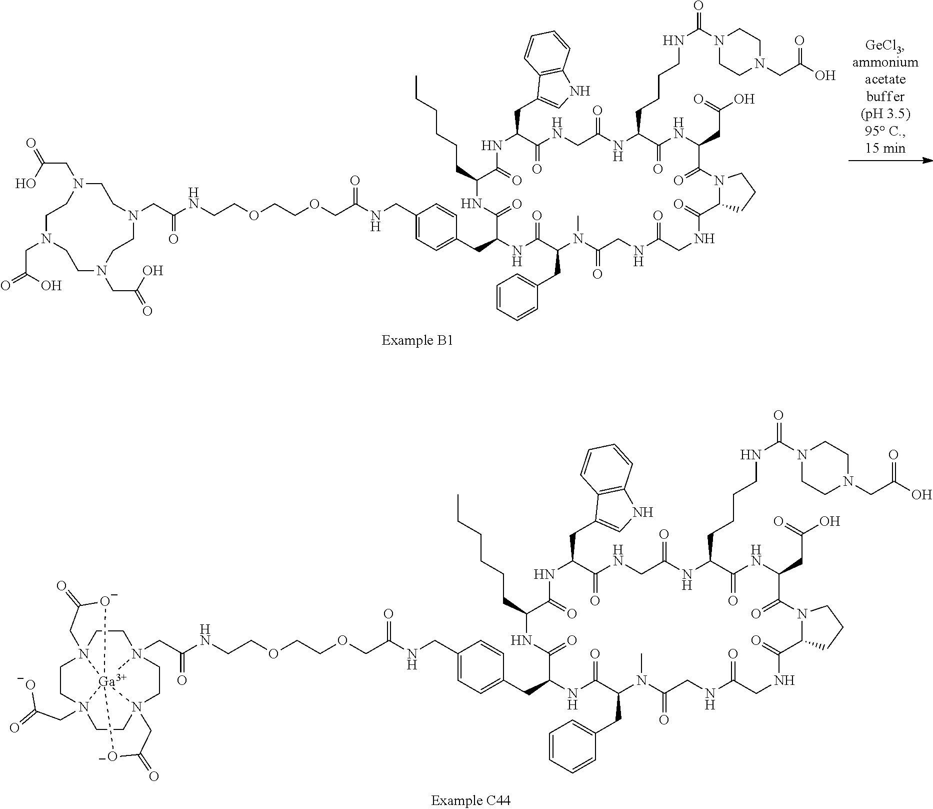

### Scheme 4.2.1. Synthesis of Example C44

Example B1 (53.40 mg, 1 eq., 21.81 mol, TFA salt (different batch than the one described in 0)) was dissolved in ammonium acetate buffer pH 3.5 (8.725 mL, 50 mM) then treated with 5 M GaCl₃ in ammonium acetate buffer pH 3.5 (8.725 μL, 5.000 M, 2.0 eq., 43.62 mol). The clear reaction mixture was stirred at 95° C. for 15 min. The product was isolated by preparative HPLC (Column: XBridge BEH C18 OBD Prep Column, 130 Å, 5 μm, 30 mm×250 mm; 30 mL/min; Eluent A: H₂O+0.08% NH4HCO₃ and eluent B: ACN). Pure fractions were combined and lyophilized to afford Example C44 (36.0 mg, 18 μmol, 82% yield) as a white solid. LCMS Method P-2, t_{R}=6.91 min, [M+H]⁺=1943.8.

### 4.2.2 Synthesis of (Cyclo{L-c-aspartyl-D-prolylglycylglycyl-N-methyl-L-phenylalanyl-4-(13-{7-(carboxymethyl)-4,10-bis[(carboxy-κO)methyl]-1,4,7,10-tetraazacyclododecan-1-yl-κ⁴N¹,N⁴,N⁷,N¹⁰}-3,12-dioxo-5,8-dioxa-2,11-diazatridecan-1-yl)-L-phenylalanyl-O-propyl-L-homoseryl-L-tryptophylglycyl-N⁶-[4-(carboxymethyl)piperazine-1-carbonyl]-L-lysyl})gallium, Example C52 (Example B2 labelled with Ga, TFA salt)

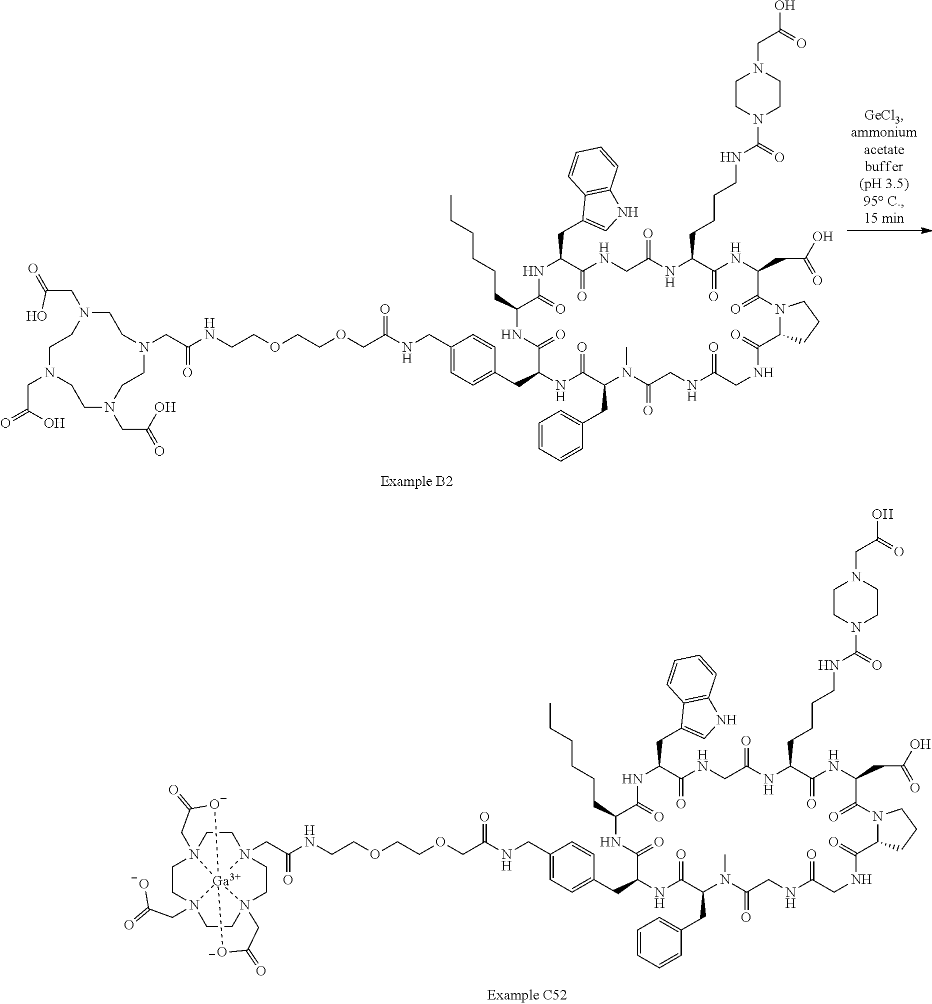

### Scheme 4.2.2. Synthesis of Example C52

Example B2 (10.00 mg, 1 eq., 4.281 mol, TFA salt) was dissolved in ammonium acetate buffer pH 3.5 (700.0 μL, 50.0 mmolar) and DMF (0.100 mL), then treated with 1 M GaCl₃ in ammonium acetate buffer pH 3.5 (15.00 μL, 1.000 molar, 3.504 eq., 15.00 mol). The clear reaction mixture was stirred at 95° C. for 15 min. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example C52 (6.3 mg, 2.9 μmol, 67% yield, TFA salt) as a white solid. LCMS method P-1, t_{R}=3.02 min, [M+H]⁺=1945.8.

The following examples (see Table 4.2) were synthesized in analogy to Example C44 and Example C52:

TABLE 4.2
Examples synthesized in analogy to
Example C44 and Example C52

| Example | | |
|---|---|---|
| No. | Description | LCMS |
| C38 | Example B30 | Method P-1, t_{R} = 3.88 min, |
| | labelled with Ga | [M + H]⁺ = 1751.7 |
| C39 | Example B32 | Method P-1, t_{R} = 3.15 min, |
| | labelled with Ga | [M + H]⁺ = 2017.8 |
| C40 | Example B33 | Method P-1, t_{R} = 3.34 min, |
| | labelled with Ga | [M + H]⁺ = 1916.8 |
| C41 | Example B34 | Method P-1, t_{R} = 2.98 min, |
| | labelled with Ga | [M + H]⁺ = 1844.8 |
| C42 | Example B38 | Method P-1, t_{R} = 3.8 min, |
| | labelled with Ga | [M + H]⁺ = 1914.8 |
| C43 | Example B40 | Method P-1, t_{R} = 3.46 min, |
| | labelled with Ga | [M + H]⁺ = 1842.8 |
| C45 | Example B6 | Method P-1, t_{R} = 3.16 min, |
| | labelled with Ga | [M + H]⁺ = 2017.9 |
| C46 | Example B14 | Method P-1, t_{R} = 3.22 min, |
| | labelled with Ga | [M + H]⁺ = 1929.8 |
| C47 | Example B13 | Method P-1, t_{R} = 3.63 min, |
| | labelled with Ga | [M + H]⁺ = 1983.9 |
| C48 | Example B28 | Method P-1, t_{R} = 2.73 min, |
| | labelled with Ga | [M + H]⁺ = 1944.8 |
| C49 | Example B16 | Method P-1, t_{R} = 3.79 min, |
| | labelled with Ga | [M + H]⁺ = 1794.7 |
| C50 | Example B3 | Method P-1, t_{R} = 3.27 min, |
| | labelled with Ga | [M + H]⁺ = 1814.8 |
| C51 | Example B23 | Method P-1, t_{R} = 3.76 min, |
| | labelled with Ga | [M + H]⁺ = 1679.7 |

### 4.3 Labelling with Natural Fluorine (Via NOTA-AlF)

### 4.3.1 Synthesis of [2,2′-(7-{1-[4-({(9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxodotriacontahydropyrrolo[1,2-a][1,4,7,10,13,16,19,22,25,28]decaazacyclotriacontin-12-yl}methyl)phenyl]-3,12-dioxo-5,8-dioxa-2,11-diazatridecan-13-yl}-1,4,7-triazonane-1,4-diyl-κ³,N¹,N⁴,N⁷)di(acetato-κO)(3-)](fluorido)aluminium, Example C54 (Example B40 labelled with AlF, TFA salt)

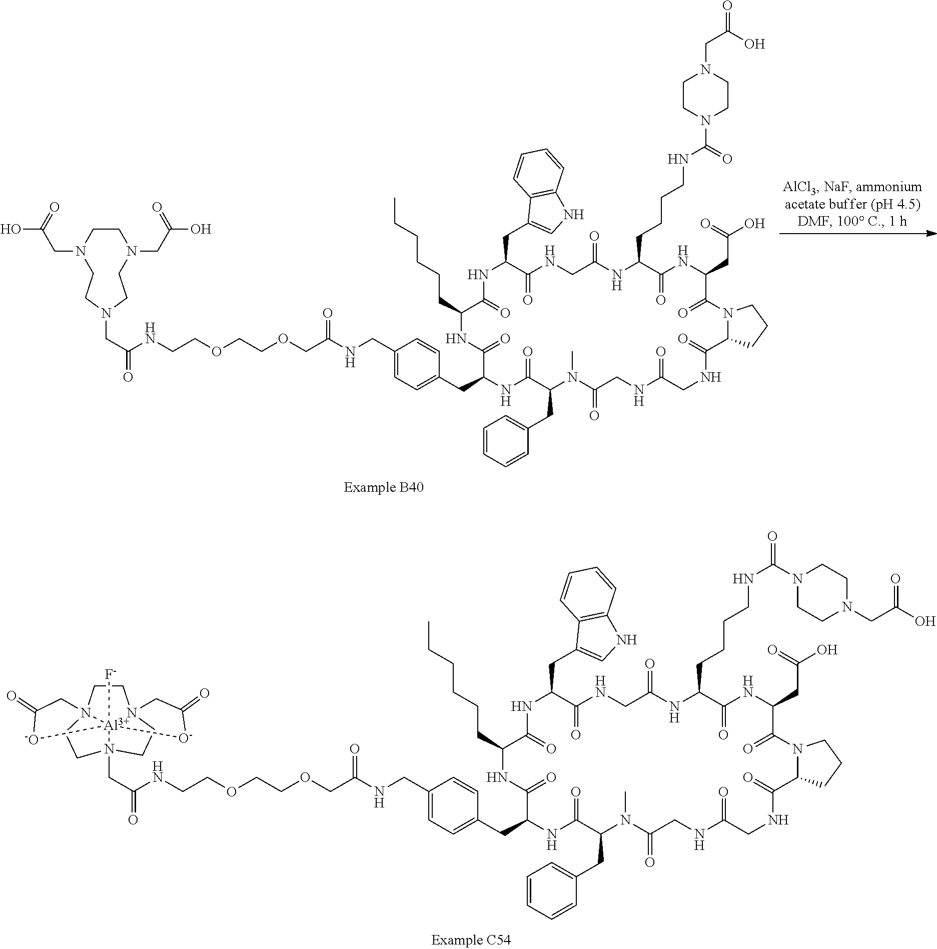

### Scheme 4.3.1. Synthesis of Example C54

To the Example B40 (1.80 mg, 1 eq., 0.898 mol, TFA salt) were added 50 mM NaF in ammonium acetate buffer pH 4.5 (98.75 μL, 50 mM, 5.5 eq., 4.938 mol), 2 mM AlCl₃ in ammonium acetate buffer pH 4.5 (493.8 μL, 2 mM, 1.1 eq., 0.988 mol), followed by DMF (0.100 mL). The reaction mixture was stirred at 100° C. for 1 h. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example C54 (0.3 mg, 0.14 μmol, 16% yield, TFA salt) as a white solid. LCMS method P-1, t_{R}=3.80 min, [M+H]⁺=1820.9.

The following example (see Table 4.3) were synthesized in analogy to Example C54:

TABLE 4.3
Example synthesized in analogy to Example C54

| Example | | |
|---|---|---|
| No. | Description | LCMS |
| C53 | Example B34 labelled | Method P-1, |
| | with AlF | t_{R} = 3.39 min, [M + H]⁺ = |
| | | 1822.8 |

### 4.4 Incorporation of Natural Fluorine Via a Prosthetic Group

### 4.4.1 Synthesis of 2-((9S,12S,15S,18S,24S,27S,32aR)-18-((1H-indol-3-yl)methyl)-9-benzyl-24-(4-(4-(carboxymethyl)piperazine-1-carboxamido)butyl)-12-(4-(12-(6-fluoropyridin-3-yl)-3,12-dioxo-5,8-dioxa-2,11-diazadodecyl)benzyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-27-yl)acetic acid TFA, (Example C55, TFA salt)

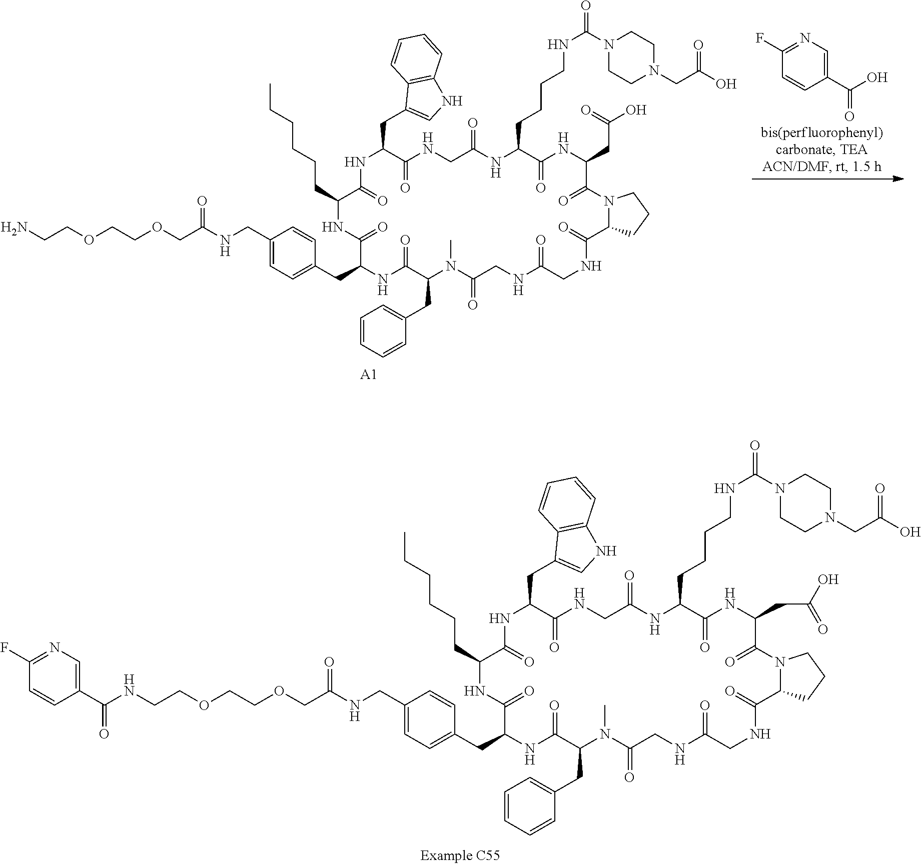

### Scheme 4.4.1. Synthesis of Example C55 (Incorporation of Natural Fluorine Via a Prosthetic Group)

To a solution of 6-fluoronicotinic acid (24.61 mg, 6.0 eq., 174.4 mol) in ACN (1.600 mL) was added triethylamine (70.61 mg, 97.3 μL, 24 eq., 697.8 mol) and bis(perfluorophenyl) carbonate (91.67 mg, 8.0 eq., 232.6 μmol). The resulting solution was stirred at rt for 1 h and an aliquot (320 L) added to the solution of A1 (50.00 mg, 1 eq., 29.07 mol, TFA salt) and triethylamine (23.54 mg, 32.4 μL, 8.0 eq., 232.6 mol) in DMF (1.000 mL). The reaction mixture was stirred at rt for 30 minutes. The product was isolated by preparative HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford C55 (38 mg, 22 μmol, 74% yield, TFA salt) as a white solid. LCMS method P-1, t_{R}=4.35 min, [M+H]⁺=1614.7.

### 4.4.2 Synthesis of 2-((9S,12S,15S,18S,24S,27S,32aR)-18-((1H-indol-3-yl)methyl)-9-benzyl-24-(4-(4-(carboxymethyl)piperazine-1-carboxamido)butyl)-12-(4-(12-(6-fluoropyridin-3-yl)-3,12-dioxo-5,8-dioxa-2,11-diazadodecyl)benzyl)-8-methyl-1,4,7,10,13,16,19,22,25,28-decaoxo-15-(2-propoxyethyl)dotriacontahydropyrrolo[1,2-a][1,4,7,10,13,16,19,22,25,28]decaazacyclotriacontin-27-yl)acetic acid, (Example C56, TFA salt)

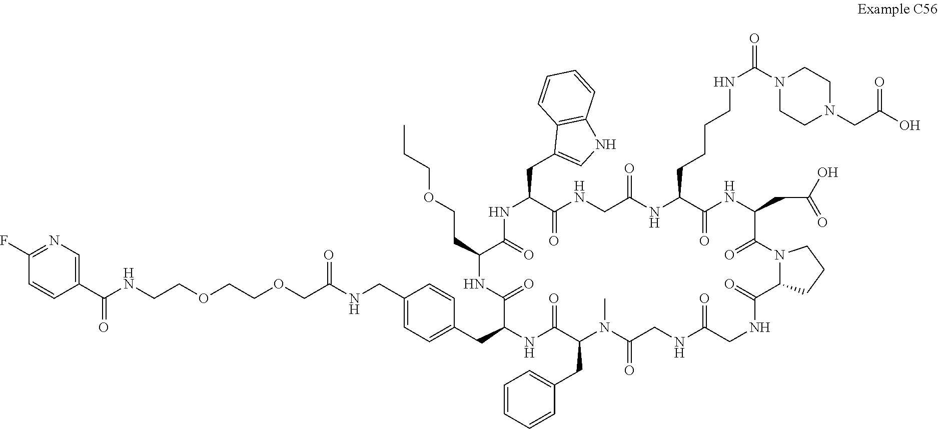 [SMILES: CCCOCC[C@@H]1NC(=O)[C@H](Cc2ccc(CNC(=O)COCCOCCNC(=O)c3ccc(F)nc3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

Example C56 was synthesized in analogy to Example C55 from A2. LCMS Method P-1, t_{R}=3.93 min [M+H]⁺=1616.8

### 4.5 Incorporation of Natural Fluorine Via Trimethylammonium-Pyridine Precursors

### 4.5.1 Synthesis of 5-((2-(2-(2-((4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-12-yl)methyl)benzyl)amino)-2-oxoethoxy)ethoxy)ethyl)carbamoyl)-N,N,N-trimethylpyridin-2-aminium (Example C57, TFA salt)

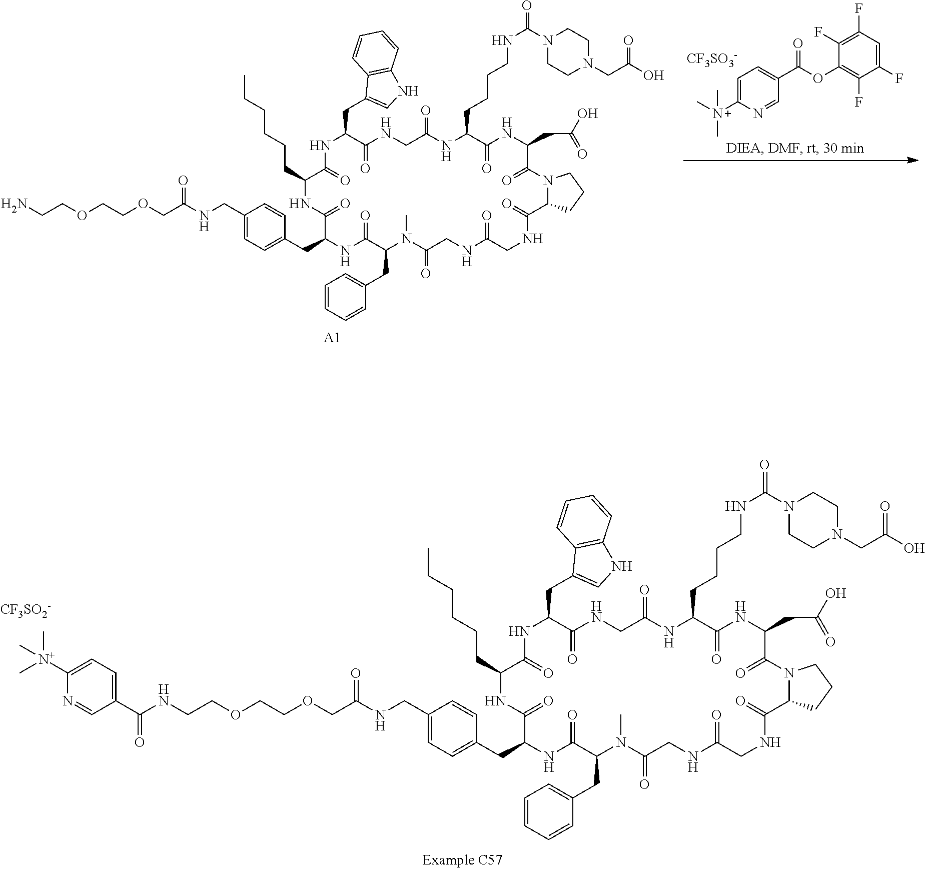

### Scheme 4.5.1. Synthesis of Example C57 (synthesis of a trimethylammonium-pyridine precursor)

To the solution of A1 (30.00 mg, 1 eq., 17.44 mol, TFA salt) in DMF (0.800 mL) was added DIEA (4.509 mg, 6.08 μL, 2 eq., 34.89 mol). The reaction mixture was stirred at rt for 2 minutes and then N,N,N-trimethyl-5-((2,3,5,6-tetrafluorophenoxy)carbonyl)pyridin-2-aminium trifluoromethanesulfonate (10.01 mg, 1.2 eq., 20.93 mol) was added. The reaction mixture was stirred at rt for 30 minutes. The product was isolated by preparative HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford C57 (11.8 mg, 6.1 μmol, 35% yield, TFA salt) as a white solid. LCMS method P-1, t_{R}=3.59 min, [M+H]⁺=1653.9.

### 4.5.2 Synthesis of 5-((2-(2-(2-((4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-8-methyl-1,4,7,10,13,16,19,22,25,28-decaoxo-15-(2-propoxyethyl)dotriacontahydropyrrolo[1,2-a][1,4,7,10,13,16,19,22,25,28]decaazacyclotriacontin-12-yl)methyl)benzyl)amino)-2-oxoethoxy)ethoxy)ethyl)carbamoyl)-N,N,N-trimethylpyridin-2-aminium (Example C58, TFA salt)

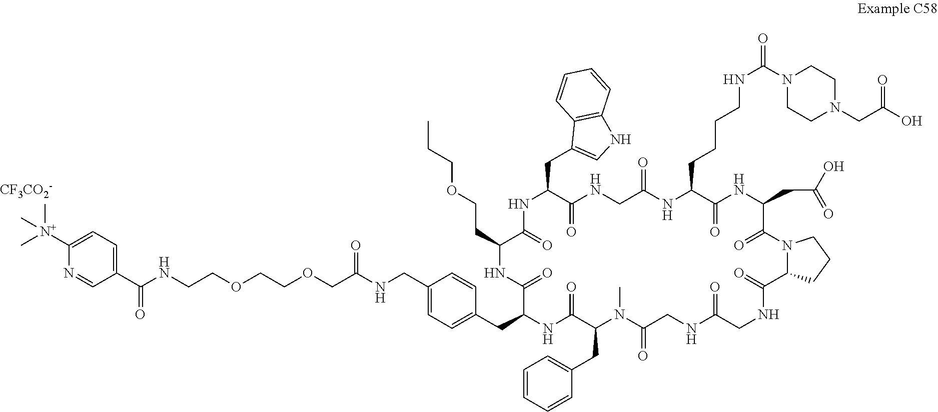 [SMILES: "CF3CO2-" ; CCCOCC[C@@H]1NC(=O)[C@H](Cc2ccc(CNC(=O)COCCOCCNC(=O)c3ccc([N+](C)(C)C)nc3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]

Example C58 was synthesized in analogy to Example C57 from A2. LCMS Method P-1, t_{R}=3.15 min [M+H]⁺=1655.8

### 4.5.3 Synthesis of 2-((9S,12S,15S,18S,24S,27S,32aR)-18-((1H-indol-3-yl)methyl)-9-benzyl-24-(4-(4-(carboxymethyl)piperazine-1-carboxamido)butyl)-12-(4-(12-(6-fluoropyridin-3-yl)-3,12-dioxo-5,8-dioxa-2,11-diazadodecyl)benzyl)-8-methyl-1,4,7,10,13,16,19,22,25,28-decaoxo-15-(2-propoxyethyl)dotriacontahydropyrrolo[1,2-a][1,4,7,10,13,16,19,22,25,28]decaazacyclotriacontin-27-yl)acetic acid (Example C56, TFA salt)

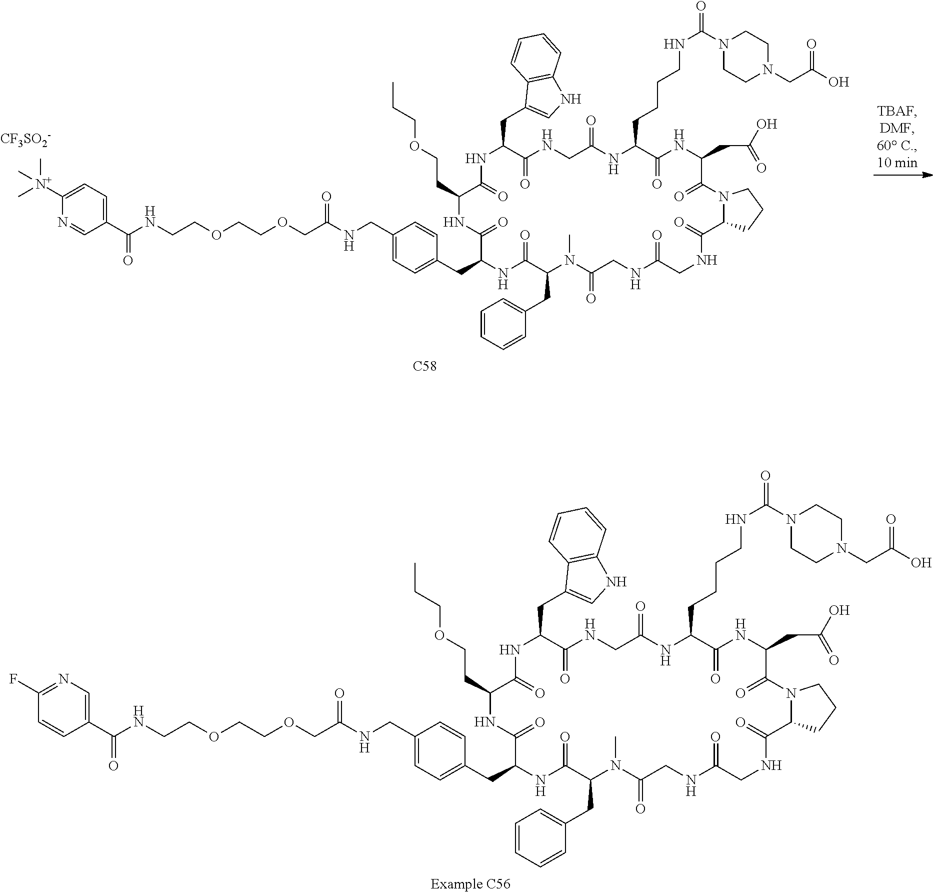 [SMILES: "CF3SO2-" ; CCCOCC[C@@H]1NC(=O)[C@H](Cc2ccc(CNC(=O)COCCOCCNC(=O)c3ccc([N+](C)(C)C)nc3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O ; "C58" ; CCCOCC[C@@H]1NC(=O)[C@H](Cc2ccc(CNC(=O)COCCOCCNC(=O)c3ccc(F)nc3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O ; "Example C56"]

### Scheme 4.5.3. Synthesis of Example C56 (incorporation of natural fluorine via a trimethylammonium-pyridine precursor)

To a solution of C58 (25.000 mg, 1 eq., 13.263 mol, TFA salt) in DMF (0.500 mL) was added TBAF solution (0.1 M in THF) (6.9358 mg, 265.26 μL, 0.1 M, 2 eq., 26.526 mol). The reaction mixture was stirred at 60 C for 10 minutes. The product was isolated by preparative HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford C56 (12 mg, 6.2 μmol, 47% yield, TFA salt) as a white solid. LCMS method P-1, t_{R}=3.97 min, [M+H]⁺=1616.8.

### 5 Varia (Examples D, F and M)

### 5.1.1 Synthesis of {2-[(1E,3E,5E)-5-{1-[6-({4-[(9S,12S,15S,18S,24S,27S,32aR)-9-Benzyl-27-(carboxymethyl)-15-hexyl-18-[(1H-indol-3-yl)methyl]-8-methyl-1,4,7,10,13,16,19,22,25,28-decaoxo-12-({4-[3-oxo-12-(oxo-κO)-13-{4,7,10-tris[(carboxy-κO)methyl]-1,4,7,10-tetraazacyclododecan-1-yl-κ⁴N¹,N⁴,N⁷,N¹⁰}-5,8-dioxa-2,11-diazatridecan-1-yl]phenyl}methyl)dotriacontahydropyrrolo[1,2-a][1,4,7,10,13,16,19,22,25,28]decaazacyclotriacontin-24-yl]butyl}amino)-6-oxohexyl]-3,3-dimethyl-5-sulfo-1,3-dihydro-2H-indol-2-ylidene}penta-1,3-dien-1-yl)-1-ethyl-3,3-dimethyl-5-sulfo-3H-indol-1-iumato(4-)}(Example D1, TFA salt)

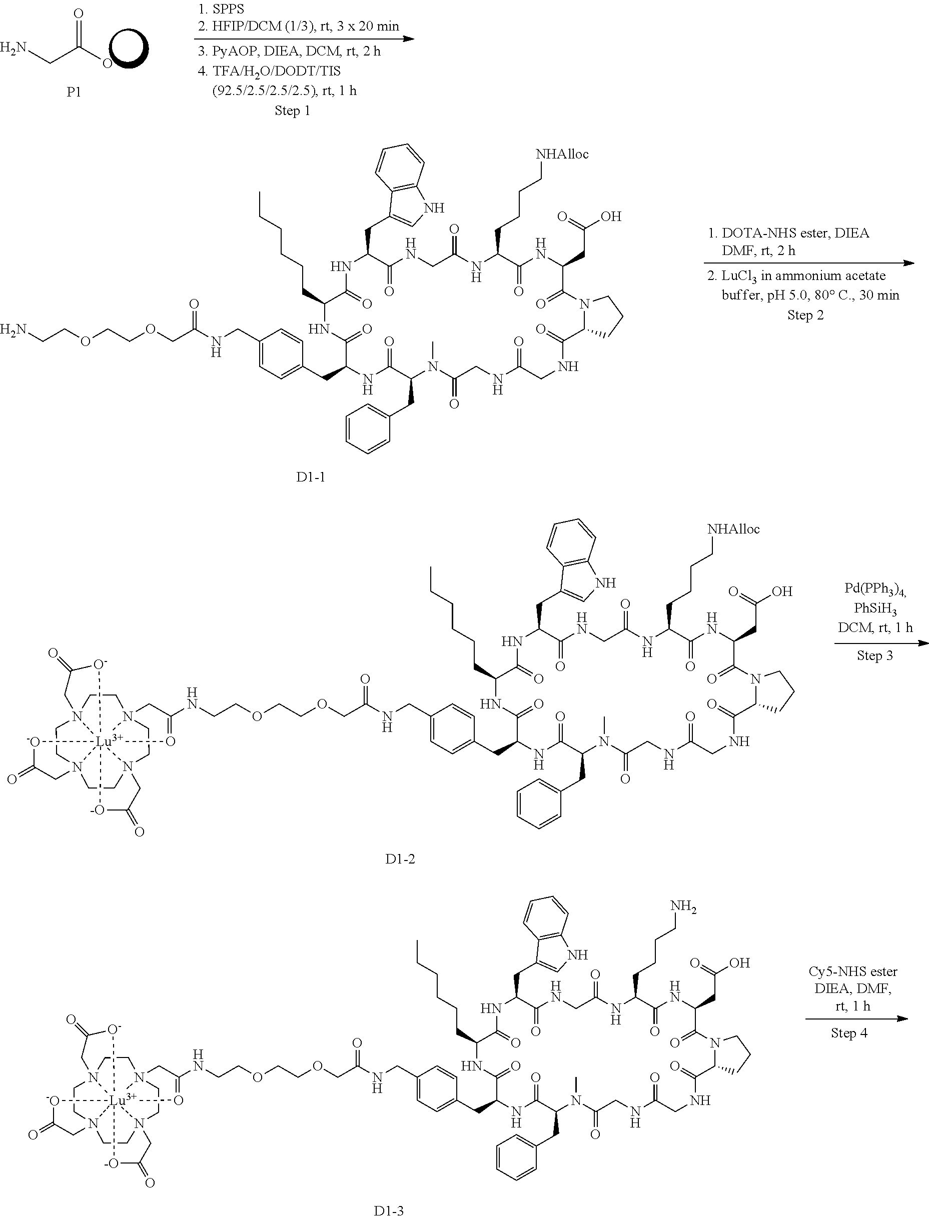

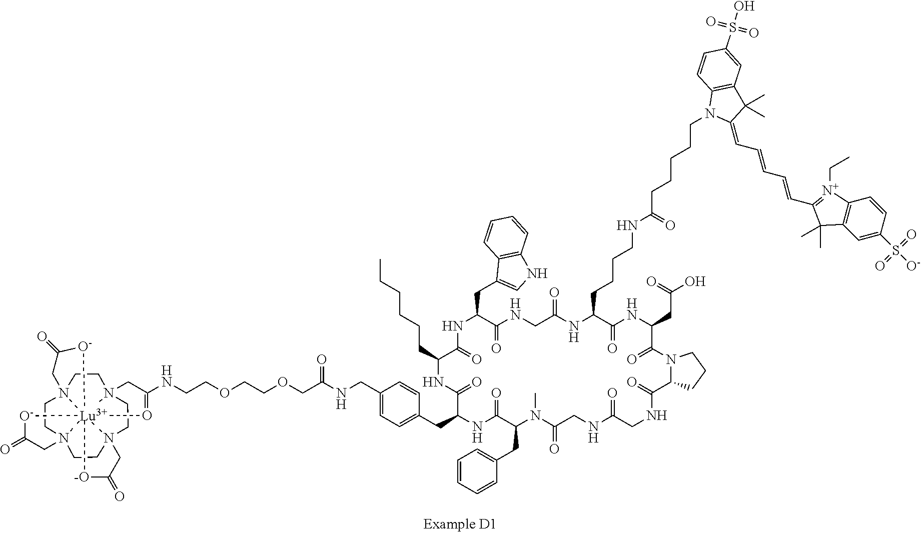

### Scheme 5.1.1. Synthesis of Example D1

### Step 1. D1-1

Step 1-1: The assembly of the linear peptide was done on the CEM Liberty Prime Synthesizer using commercial preloaded resin P1 (loading 0.79 mmol/g, 127 mg, 0.100 mmol) (corresponding to A¹-resin). All amino acid couplings were performed following the same method: After automated addition of Fmoc-amino acid (0.25 M in DMF, 2.5 eq.), Oxyma Pure®+DIEA (0.25 M Oxyma Pure® and 0.1 M DIEA in DMF, 5 eq. of Oxyma Pure®, 2 eq of DIEA), and DIC (2 M in DMF, 10 eq.) the suspension was mixed at rt for 1 h. The coupling step was repeated. Fmoc removal was performed using pyrrolidine/DMF (1/3) (22.8 eq), automatically added to the previous amino acid coupling reaction mixture at rt for 10 min. The deprotection step was repeated. The amino acids used are summarized in Table 5.1.

TABLE 5.1

| Synthesis | Amino acid | |
|---|---|---|
| cycle | residue* | Amino acid |
| 1 | A¹⁰ | Fmoc-D-Pro-OH |
| 2 | A⁹ | Fmoc-Asp(OMpe)-OH |
| 3 | A⁸ | Fmoc-Lys(Alloc)-OH |
| 4 | A⁷ | Fmoc-Gly-OH |
| 5 | A⁶ | Fmoc-Trp(Boc)-OH |
| 6 | A⁵ | Fmoc-Aoc-OH |
| 7 | A⁴ | BB40 |
| 8 | A³ | Fmoc-N-Me-Phe-OH |
| 9 | A² | Fmoc-Gly-OH |

*Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added HFIP/DCM (1/3) (4 mL) and the resin was shaken at rt for 20 min. The cleavage solution was filtered off and the procedure was repeated (2×). The resin was washed with HFIP/DCM (1/3) (30 mL). The combined cleavage and washing solutions were concentrated to dryness in vacuo. The resin was treated with toluene and the resulting mixture was concentrated to dryness in vacuo (2×). The residue was dissolved in tert-BuOH (6 mL) and H₂O (4 mL) and lyophilized.

Step 1-3: The residue from Step 1-2 was dissolved in DCM (100 mL). Then PyAOP (103.79 mg, 2 eq., 199.06 gmol), followed by DIEA (128.64 mg, 173 μL, 10 eq., 995.30 μmol) were added. The reaction mixture was stirred at rt for 2 h, then concentrated to dryness in vacuo. The residue was redissolved in tert-BuOH (3 mL) and H₂O (3 mL) and lyophilized.

Step 1-4: The residue from Step 1-3 was dissolved in TFA/H₂O/DODT/TIS (92.5/2.5/2.5/2.5) (5 mL), and the reaction mixture was stirred at rt for 1 h. The crude peptide was precipitated with cold diethyl ether (35 mL). The suspension was centrifuged, and the solvent was decanted. The product was isolated by preparative HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford D1-1 (37 mg, 25 μmol, 25% yield, TFA salt) as a white solid. LCMS method P-1, t_{R}=4.36 min, [M+H]⁺=1405.7.

### Step 2. D1-2

Step 2-1: D1-1 (61.00 mg, 1 eq., 40.14 mol, TFA salt) (two batches of D1-1 synthesized through different campaigns were combined) was dissolved in DMF (1.50 mL), then DOTA-NHS ester, (30.53 mg, 1 eq., 40.14 μmol, trifluoroacetate-hexafluorophosphate) and DIEA (41.51 mg, 55.9 μL, 8 eq., 321.1 mol) were added. The reaction mixture was shaken at rt for 2 h.

Step 2-2: Then 100 mM LuCl₃ in ammonium acetate buffer pH 5.0 (1.000 mL, 100 mM, 2.491 eq., 100.0 μmol) was added. The reaction mixture was shaken at 80° C. for 30 min. The product was isolated by preparative HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford D1-2 (74 mg, 35 μmol, 88% yield, TFA salt) as a white solid. LCMS method P-1, t_{R}=4.57 min, [M+H]⁺=1963.8.

### Step 3. D1-3

D1-2 (60.00 mg, 1 eq., 28.87 mol, TFA salt) was dissolved in DCM (3.00 mL). The suspension was treated with phenylsilane (46.87 mg, 53.7 μL, 15 eq., 433.1 mol) and then 2 min later Pd(PPh₃)₄ (3.337 mg, 0.1 eq., 2.887 mol) was added. The reaction mixture was stirred at rt for 1 h, then concentrated to dryness in vacuo. The product was isolated by preparative HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford D1-3 (51 mg, 24 μmol, 83% yield, TFA salt) as a white solid. LCMS method P-1, t_{R}=3.58 min, [M+H]⁺=1879.8.

### Step 4. Example D1

To the solution of D1-3 (51.00 mg, 1 eq., 24.19 mol, TFA salt) in DMF (0.500 mL) at rt were added Cy5-NHS ester (CAS 146368-14-1, 21.92 mg, 1.2 eq., 29.03 mol) and DIEA (12.51 mg, 16.9 μL, 4 eq., 96.78 mol). The reaction mixture was stirred at rt for 1 h. The reaction mixture was cooled down to 0° C. and diluted with ACN/H₂O (1/1) (1.5 mL). The product was isolated by preparative HPLC (Column: SunFire C18 OBD Prep Column, 100 Å, 5 μm, 30 mm×100 mm; 30 mL/min; Eluent A: H₂O+0.1% TFA and eluent B: ACN). Pure fractions were combined and lyophilized to afford Example D1 (46 mg, 17 μmol, 71% yield, TFA salt) as a blue solid. LCMS method P-1, t_{R}=4.57 min, [M+2H]²⁺=1259.5.

### 5.1.2 Synthesis of {2-[(1E,3E,5E)-5-{1-[6-({4-[(3S,9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxo-12-({4-[3-oxo-12-(oxo-κO)—13-{4,7,10-tris[(carboxy-κO)methyl]-1,4,7,10-tetraazacyclododecan-1-yl-κ⁴N¹,N⁴,N⁷,N¹⁰}-5,8-dioxa-2,11-diazatridecan-1-yl]phenyl}methyl)dotriacontahydropyrrolo[1,2-a][1,4,7,10,13,16,19,22,25,28]decaazacyclotriacontin-3-yl]butyl}amino)-6-oxohexyl]-3,3-dimethyl-5-sulfo-1,3-dihydro-2H-indol-2-ylidene}penta-1,3-dien-1-yl]-1-ethyl-3,3-dimethyl-5-sulfo-3H-indol-1-iumato(4-)}lutetium (Example D2, TFA salt)

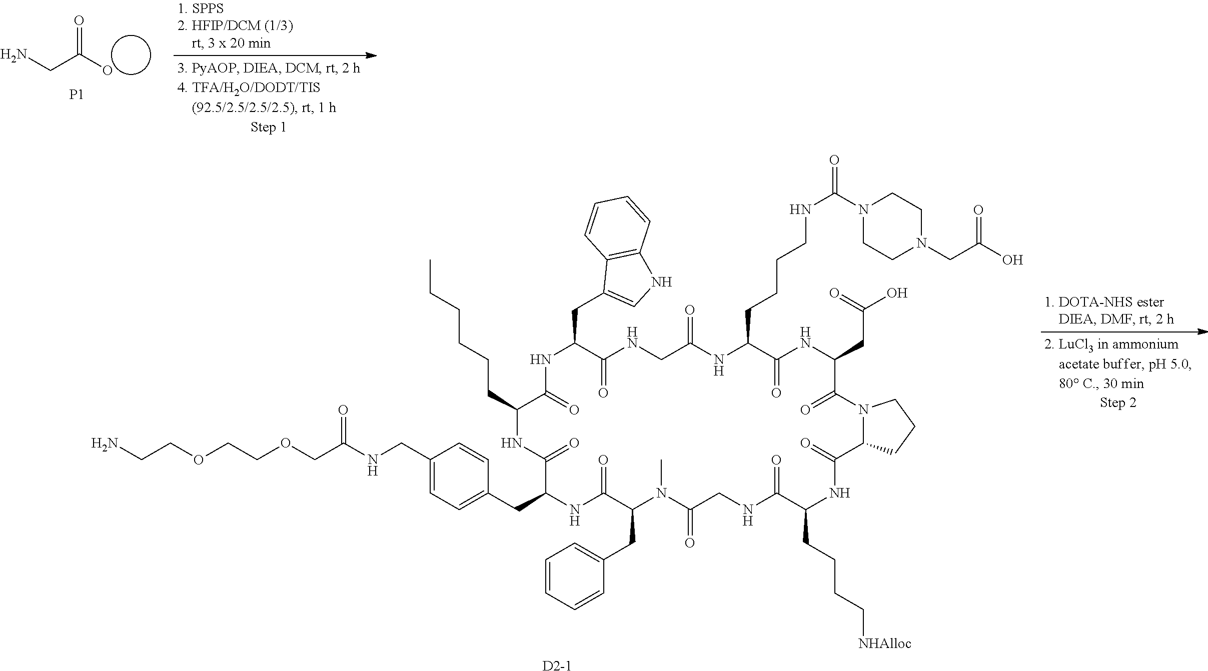

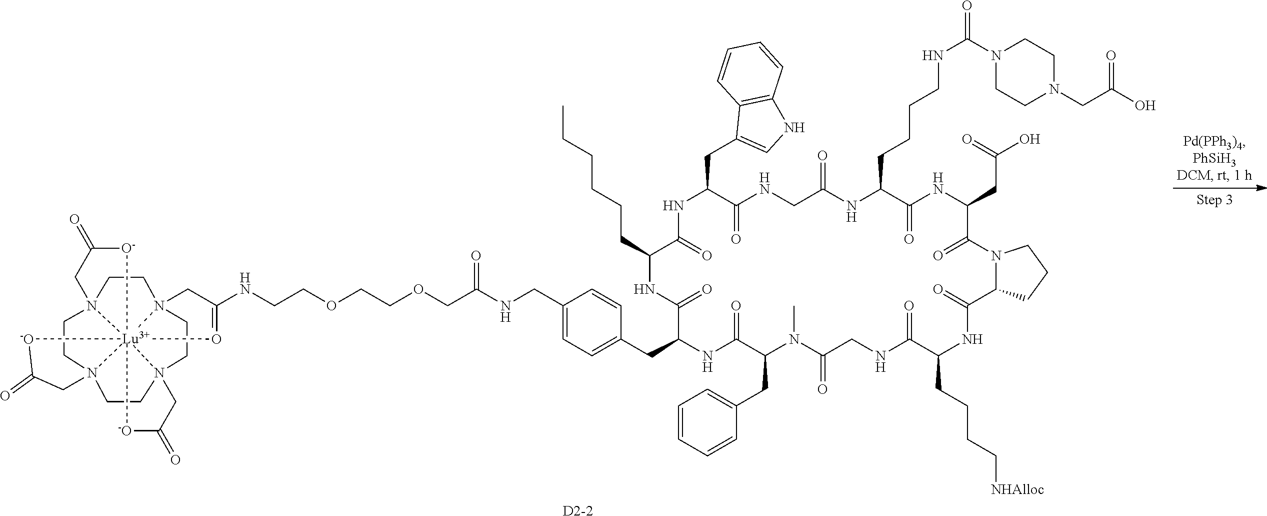

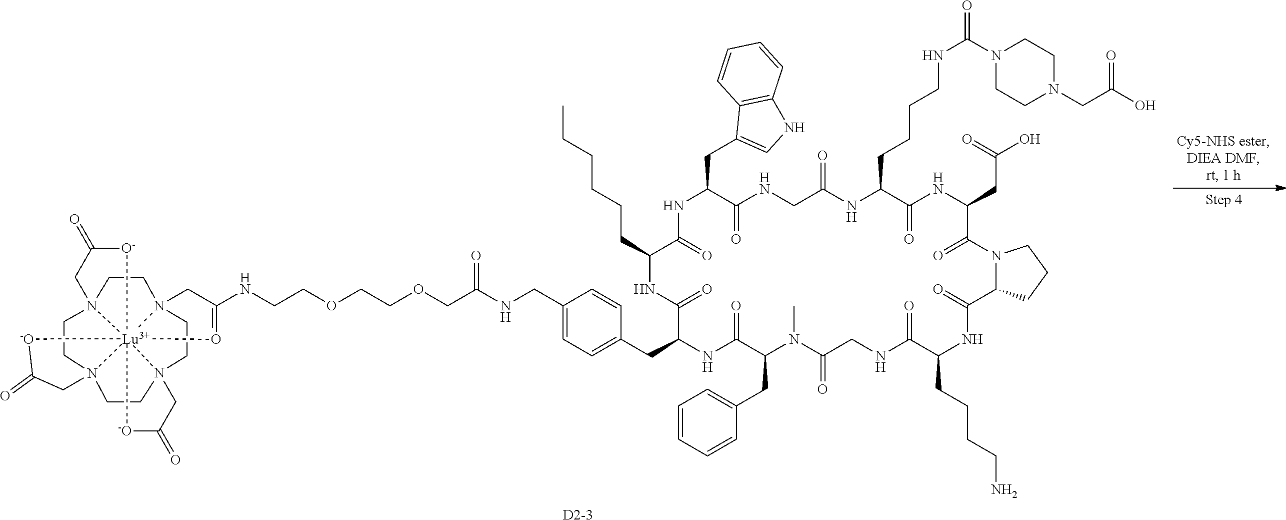

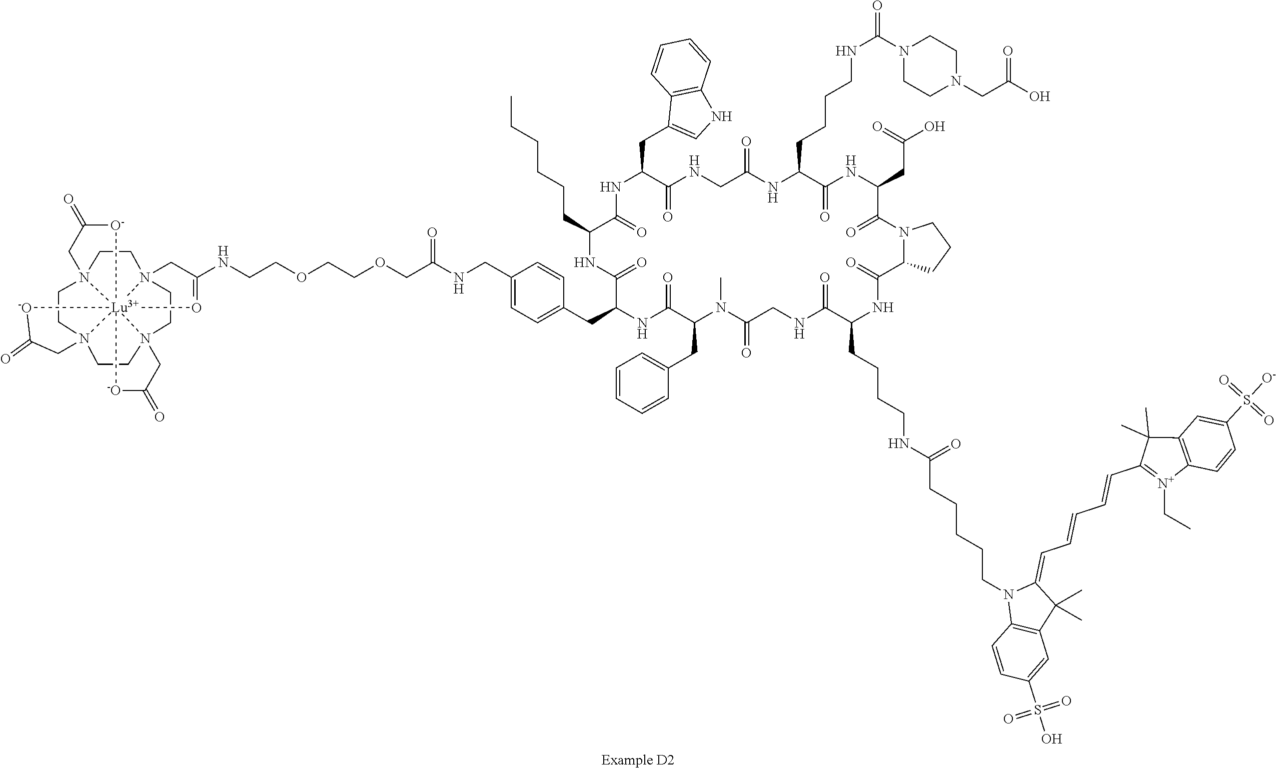

### Scheme 5.1.2. Synthesis of Example D2

### Step 1. D2-1

Step 1-1: The assembly of the linear peptide was done on the CEM Liberty Prime Synthesizer using commercial preloaded resin P1 (loading 0.79 mmol/g, 253 mg, 0.200 mmol) (corresponding to A²-resin). All amino acid couplings were performed following the same method: After automated addition of Fmoc-amino acid (0.25 M solution in DMF, 2 mL, 2.5 eq.), Oxyma Pure®+DIEA (0.25 M Oxyma Pure® and 0.1 M DIEA in DMF, 4 mL, 5 eq. of Oxyma Pure®, 2 eq of DIEA), and DIC (2 M solution in DMF, 1 mL, 10 eq.) the suspension was mixed at 50° C. for 20 min. The coupling step was repeated. Fmoc removal was performed using a solution of pyrrolidine/DMF (1/3) (22.8 eq.) automatically added to the previous amino acid coupling reaction mixture at rt for 10 min. The deprotection step was repeated. The amino acids used are summarized in Table 5.2.

TABLE 5.2

| Synthesis | Amino acid | |
|---|---|---|
| cycle | residue* | Amino acid |
| 1 | A¹ | Fmoc-Lys(Alloc)-OH |
| 2 | A¹⁰ | Fmoc-D-Pro-OH |
| 3 | A⁹ | Fmoc-Asp(OMpe)-OH |
| 4 | A⁸ | BB41 |
| 5 | A⁷ | Fmoc-Gly-OH |
| 6 | A⁶ | Fmoc-Trp(Boc)-OH |
| 7 | A⁵ | Fmoc-Aoc-OH |
| 8 | A⁴ | BB40 |
| 9 | A³ | Fmoc-N-Me-Phe-OH |

*Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added HFIP/DCM (1/3) (4 mL) and the resin was shaken at rt for 20 min. The cleavage solution was filtered off and the procedure was repeated two times. The resin was washed with HFIP/DCM (1/3) (30 mL). The combined cleavage and washing solutions were concentrated to dryness in vacuo. The resin was treated with toluene and the resulting mixture was concentrated to dryness in vacuo two times. The residue was dissolved in tert-BuOH (6 mL) and H₂O (4 mL) and lyophilized (267 mg, 110 mol).

Step 1-3: The residue from Step 1-2 (263 mg, 104 μmol) was dissolved in DCM (20 mL). Then DIEA (134 mg, 180 μL, 10 eq., 1.04 mmol), followed by PyAOP (108 mg, 2 eq., 207.2 mol) were added. The reaction mixture was stirred at rt for 2 h, then concentrated to dryness in vacuo. The residue was redissolved in tert-BuOH (4 mL) and H₂O (4 mL) and lyophilized.

Step 1-4: The residue from Step 1-3 was dissolved in TFA/H₂O/DODT/TIS (92.5/2.5/2.5/2.5) (5 mL), and the reaction mixture was stirred at rt for 1 h. The crude peptide was precipitated with cold diethyl ether (35 mL). The suspension was centrifuged, and the solvent was decanted. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford D2-1 (94 mg, 45 mol, TFA salt) as a white solid. LCMS method P-1, t_{R}=3.89 min, [M+H]⁺=1646.9.

### Step 2. D2-2

Step 2-1: D2-1 (94.00 mg, 1 eq., 45 mol, TFA salt) was dissolved in DMF (1.500 mL), then DOTA-NHS ester (41.2 mg, 1.2 eq., 54.1 mol, trifluoroacetate-hexafluorophosphate) and DIEA (46.7 mg, 62.9 μL, 8 eq., 361 μmol) were added. The reaction mixture was shaken at rt for 2 h.

Step 2-2: Then 100 mM LuCl₃ in ammonium acetate buffer pH 5.0 (1.000 mL, 100 mM, 2.2 eq., 100.0 μmol) was added. The reaction mixture was shaken at 80° C. for 30 min. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford D2-2 (89 mg, 35 μmol, 69% yield, TFA salt) as a white solid. LCMS method P-1, t_{R}=4.00 min, [M+H]⁺=2205.0.

### Step 3. D2-3

D2-2 (89.0 mg, 1 eq., 35 mol, TFA salt) was dissolved in DCM (3.00 mL). The suspension was treated with phenylsilane (59.37 mg, 68.0 μL, 15.7 eq., 548.6 mol) and then 2 min later Pd(PPh₃)₄ (4.227 mg, 0.1 eq., 3.658 mol) was added. The reaction mixture was stirred at rt for 1 h, then concentrated to dryness in vacuo. The product was isolated by preparative HPLC (Column: XSelect Peptide CSH C18 OBD Prep Column, 130 Å, 5 μm, 30 mm×250 mm; 30 mL/min; Eluent A: H₂O+0.10% TFA and eluent B: ACN). Pure fractions were combined and lyophilized to afford D2-3 (96 mg, mol, 99% yield, TFA salt) as a white solid. LCMS method P-1, t_{R}=3.33 min, [M+H]⁺=2120.9.

### Step 4. Example D2

To the solution of D2-3 (96.0 mg, 1 eq., 35 mol, TFA salt) in DMF (0.500 mL) at rt were added Cy5-NHS ester (CAS 146368-14-1, 29.42 mg, 1.1 eq., 38.97 mol) and DIPEA (20.15 mg, 27.2 μL, 4.5 eq., 155.9 mol). The reaction mixture was stirred at rt for 1 h. The reaction mixture was cooled down to 0° C. and diluted with ACN/H₂O (1/1) (1.5 mL). The product was isolated by preparative HPLC (Column: SunFire C18 OBD Prep Column, 100 Å, 5 μm, 30 mm×100 mm; 30 mL/min; Eluent A: H₂O+0.10% TFA and eluent B: ACN). Pure fractions were combined and lyophilized to afford Example D2 (59 mg, 20 μmol, 57% yield, TFA salt) as a blue solid. LCMS method P-1, t_{R}=4.18 min, [M+2H]²⁺=1380.1.

### 5.1.3 Synthesis of [N⁶-{3-[(6S,9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxo-12-({4-[3-oxo-12-(oxo-κO)-13-{4,7,10-tris[(carboxy-κO)methyl]-1,4,7,10-tetraazacyclododecan-1-yl-κ⁴N¹,N⁴,N⁷,N¹⁰}-5,8-dioxa-2,11-diazatridecan-1-yl]phenyl}methyl)dotriacontahydropyrrolo[1,2-a][1,4,7,10,13,16,19,22,25,28]decaazacyclotriacontin-6-yl]propanoyl}-N²-[4-(4-iodophenyl)butanoyl]-L-lysinato(3-)]lutetium (Example F1, TFA salt)

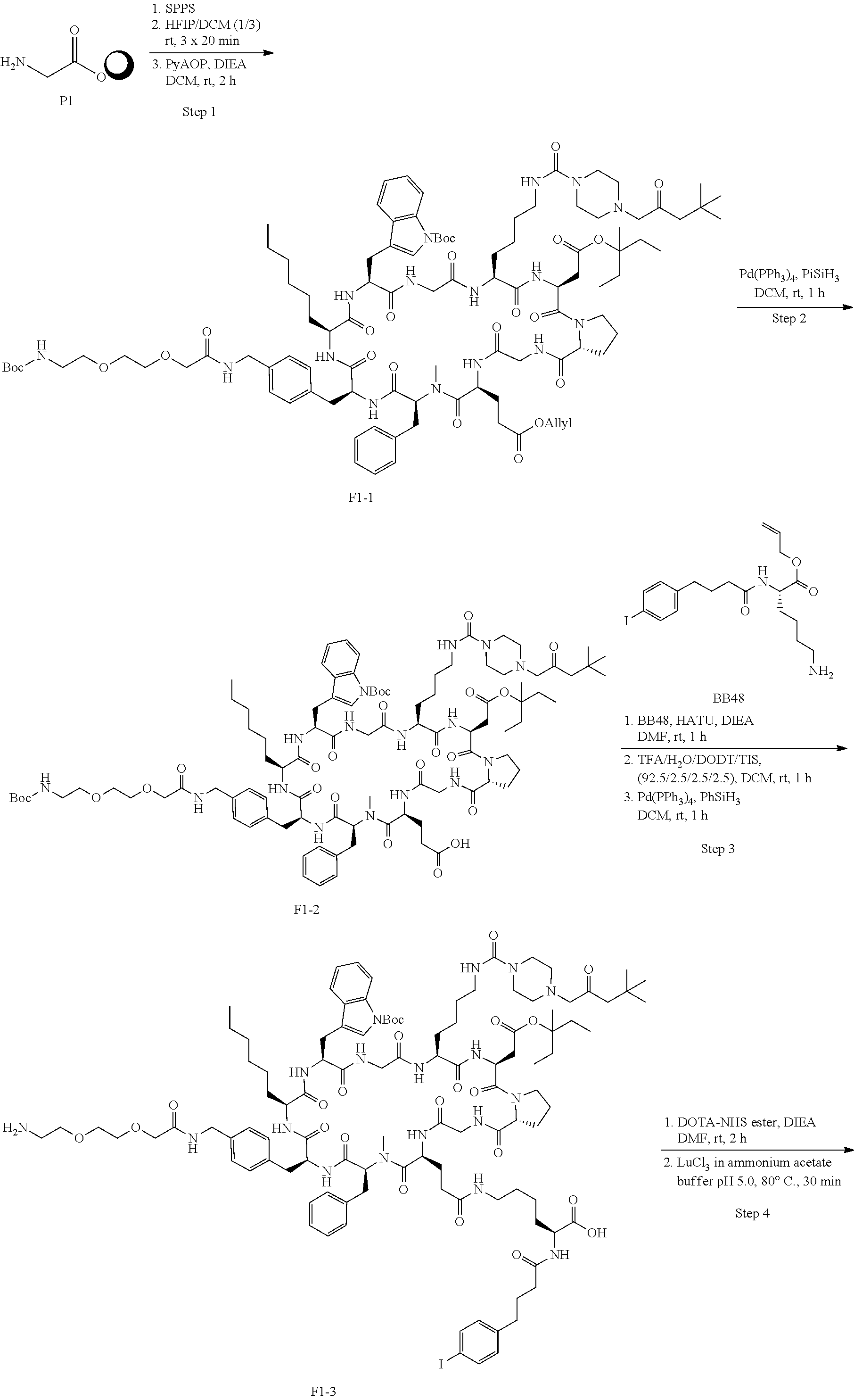

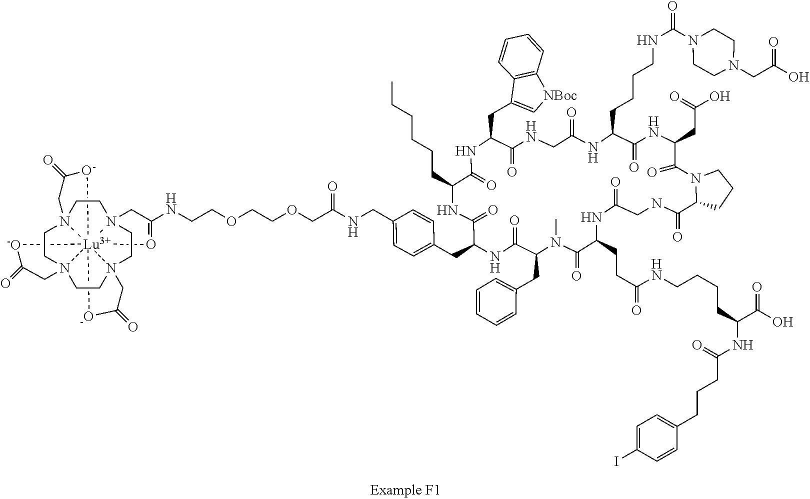

### Scheme 5.1.3. Synthesis of Example F1 (Incorporation of an Albumin Binder)

### Step 1. F1-1

Step 1-1: The assembly of the linear peptide was done on the CEM Liberty Prime Synthesizer using commercial preloaded resin P1 (loading 0.79 mmol/g, 253 mg, 1 eq., 0.200 mmol) (corresponding to A¹-resin). All amino acid couplings were performed following the same method: After automated addition of Fmoc-amino acid (0.25 M in DMF, 2 mL, 2.5 eq.), Oxyma Pure®+DIEA (0.25 M Oxyma Pure® and 0.1 M DIEA in DMF, 4 mL, 5 eq. of Oxyma Pure®, 2 eq of DIEA), and DIC (2 M in DMF, 1 mL, 10 eq.) the suspension was mixed at 50° C. for 20 min. The coupling step was repeated. Fmoc removal was performed using a solution of pyrrolidine/DMF (1/3) (22.8 eq.) automatically added to the previous amino acid coupling reaction mixture at rt for 10 min. The deprotection step was repeated. The amino acids used are summarized in Table 5.3.

TABLE 5.3

| Synthesis | Amino acid | |
|---|---|---|
| cycle | residue* | Amino acid |
| 1 | A¹⁰ | Fmoc-D-Pro-OH |
| 2 | A⁹ | Fmoc-Asp(OMpe)-OH |
| 3 | A⁸ | BB41 |
| 4 | A⁷ | Fmoc-Gly-OH |
| 5 | A⁶ | Fmoc-Trp(Boc)-OH |
| 6 | A⁵ | Fmoc-Aoc-OH |
| 7 | A⁴ | BB40 |
| 8 | A³ | Fmoc-N-Me-Phe-OH |
| 9 | A² | Fmoc-Glu(OAll)-OH |

*Refers to the corresponding amino acid residue of cyclic peptide, P, as found, e.g., in Formulae (I), (Ia), (Ib), (Ic), (Id), or in the Table of Example III.

Step 1-2: To the resin from Step 1-1 was added HFIP/DCM (1:3) (4 mL) and the resin was shaken at rt for 20 min. The cleavage solution was filtered off and the procedure was repeated two times. The resin was washed with HFIP/DCM (1/3) (30 mL). The combined cleavage and washing solutions were concentrated to dryness in vacuo. The resin was treated with toluene and the resulting mixture was concentrated to dryness in vacuo two times. The residue was dissolved in tert-BuOH (6 mL) and H₂O (4 mL) and lyophilized.

Step 1-3: The residue from Step 1-2 was dissolved in DCM (200 mL). Then DIEA (207 mg, 279 μL, 8 eq., 1.6 mmol), followed by PyAOP (209 mg, 2 eq., 400 mol) were added. The reaction mixture was stirred at rt for 2 h, then concentrated to dryness in vacuo. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford F1-1 (77 mg, 33.7 mol, TFA salt) as a white solid. LCMS method P-1, t_{R}=6.57 min, [M+H]⁺=1944.1.

### Step 2. F1-2

F1-1 (77 mg, 1 eq., 33.7 mol, TFA salt) was dissolved in DCM (3.000 mL). The suspension was treated with phenylsilane (54.6 mg, 62.6 μL, 15 eq., 505 mol) and then 2 min later Pd(PPh₃)₄(3.9 mg, 0.1 eq., 3.4 mol) was added. The reaction mixture was stirred at rt for 1 h, then concentrated to dryness in vacuo. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford F1-2 (25 mg, 11.8 μmol, 35% yield, TFA salt) as a white solid. LCMS method P-1, t_{R}=6.18 min, [M+H]⁺=1904.1.

### Step 3. F1-3

Step 3-1: BB48 (8.1 mg, 1.5 eq., 17.7 mol) was dissolved in DMF (1.000 mL). The suspension was treated with HATU (8.9 mg, 2 eq., 23.5 μmol) and DIEA (9.1 mg, 12.3 μL, 6 eq., 70.6 mol) and then 5 min later F1-2 (25.00 mg, 1 eq., 11.8 mol, TFA salt) was added. The reaction solution was stirred at rt for 1 h. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized (24 mg, 9.7 μmol, 74% yield, TFA salt) as a white solid. LCMS method P-3 t_{R}=3.14 min, [M+H]⁺=2344.2.

Step 3-2: The product from Step 3-1 was dissolved in DCM (3 mL) and TFA/H₂O/DODT/TIS (92.5/2.5/2.5/2.5) (3 mL) was added, and the reaction mixture was stirred at rt for 1 h. The crude peptide was concentrated to dryness in vacuo. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized (18 mg, 8 μmol, 61% yield, TFA salt) as a white solid. LCMS method P-3 t_{R}=2.25 min, [M+H]⁺=2003.9.

Step 3-3: The product from Step 3-2 (18 mg, 1 eq., 8.0 mol, TFA salt) was dissolved in DCM (2.000 mL). The suspension was treated with phenylsilane (8.727 mg, 10.0 μL, 10 eq., 80.64 mol) and then 2 min later Pd(PPh₃)₄(1.398 mg, 0.15 eq., 1.210 mol) was added. The reaction mixture was stirred at rt for 1 h, then concentrated to dryness in vacuo. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford F1-3 (12 mg, 5.4 μmol, 67% yield, TFA salt) as a white solid. LCMS method P-3, t_{R}=2.07 min, [M+H]⁺=1963.8.

### Step 4. Example F1

Step 4-1: F1-3 (12 mg, 1 eq., 5.4 mol, TFA salt) was dissolved in DMF (1.00 mL), then DOTA-NHS ester (4.996 mg, 1.2 eq., 6.569 mol, trifluoroacetate-hexafluorophosphate) and DIPEA (7.075 mg, 9.46 μL, 10 eq., 54.74 mol) were added. The reaction mixture was shaken at rt for 2 h.

Step 4-2: The reaction mixture was cooled down to 0° C., then 100 mM LuCl₃ in ammonium acetate buffer pH 5.0 (1.000 mL, 100 mM, 18.27 eq., 100.0 mol) was added. The reaction mixture was shaken at 80° C. for 30 min. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example F1 (12.2 mg, 4.4 μmol, 81% yield, TFA salt) as a white solid. LCMS method P-1, t_{R}=4.57 min, [M+H]⁺=2521.9.

### 5.1.4 Synthesis of [IN-{3-[(6S,9S,12S,15S,18S,24S,27S,32aR)-9-Benzyl-27-(carboxymethyl)-24-(4-{[4-(carboxymethyl)piperazine-1-carbonyl]amino}butyl)-15-hexyl-18-[(1H-indol-3-yl)methyl]-8-methyl-1,4,7,10,13,16,19,22,25,28-decaoxo-12-({4-[3-oxo-12-(oxo-κO)-13-{4,7,10-tris[(carboxy-κO)methyl]-1,4,7,10-tetraazacyclododecan-1-yl-κ⁴N¹,N⁴,N⁷,N¹⁰}-5,8-dioxa-2,11-diazatridecan-1-yl]phenyl}methyl)dotriacontahydropyrrolo[1,2-a][1,4,7,10,13,16,19,22,25,28]decaazacyclotriacontin-6-yl]propanoyl}-N⁶-[4-(4-iodophenyl)butanoyl]-L-lysinato(3-)]lutetium (Example F2, TFA salt)

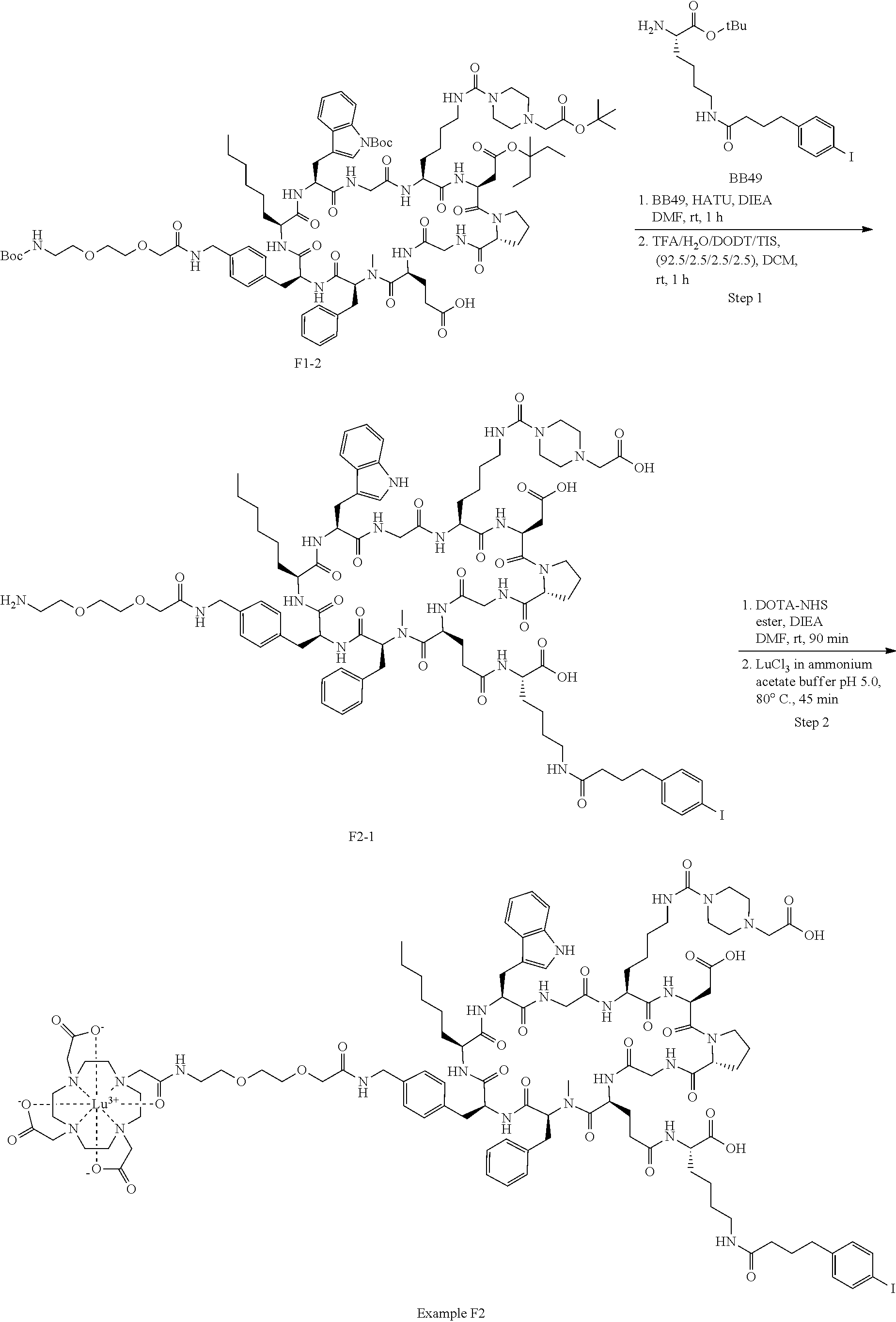

### Scheme 5.1.4. Synthesis of Example F2 (Incorporation of an Albumin Binder)

### Step 1. F2-1

Step 1-1: BB49 (30.08 mg, 3.199 eq., 63.41 μmol) was dissolved in DMF (1.000 mL). The suspension was treated with HATU (15.07 mg, 2 eq., 39.64 μmol) and DIPEA (15.37 mg, 20.7 μL, 6 eq., 118.9 μmol) and then 2 min later F1-2 (40.00 mg, 1 eq., 19.82 μmol, TFA salt) was added. The reaction solution was stirred at rt for 1 h. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford the product (38.5 mg, 15.2 μmol, 77% yield, TFA salt) as a white solid. LCMS method P-3, t_{R}=3.30 min, [M+H]⁺=2360.2.

Step 1-2: The product from Step 1-1 was dissolved in DCM (3 mL) and TFA/H₂O/DODT/TIS (92.5/2.5/2.5/2.5) (3 mL) was added. The reaction mixture was stirred at rt for 1 h, then concentrated to dryness in vacuo. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford F2-1 (33 mg, 14.8 μmol, 97% yield, TFA salt) as a white solid. LCMS method P-3, t_{R}=2.04 min, [M+H]⁺=1963.8.

### Step 2: Example F2

Step 2-1. F2-1 (17 mg, 1 eq., 7.755 mol, TFA salt) was dissolved in DMF (1.00 mL), then DOTA-NHS ester (7.077 mg, 1.2 eq., 9.306 mol, trifluoroacetate-hexafluorophosphate) and DIEA (10.02 mg, 13.4 μL, 10 eq., 77.55 μmol) were added. The reaction mixture was shaken at rt for 90 min.

Step 2-2: The reaction mixture was cooled down to 0° C., then 100 mM LuCl₃ in ammonium acetate buffer pH 5.0 (1.000 mL, 100 mM, 12.89 eq., 100.0 mol) was added. The reaction mixture was shaken at 80° C. for 45 min. The product was isolated by preparative HPLC (Column: XSelect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example F2 (14.8 mg, 5.3 μmol, 69% yield, TFA salt) as a white solid. LCMS method P-1, t_{R}=4.62 min, [M+H]⁺=2521.9.

### 5.1.5 Synthesis of 2,2′,2″-(10-(1-(4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-12-yl)methyl)phenyl)-14-(1-(4-(((9S,12S,15S,18S,24S,27S,32aR)-18-((1H-indol-3-yl)methyl)-9-benzyl-27-(carboxymethyl)-24-(4-(4-(carboxymethyl)piperazine-1-carboxamido)butyl)-15-hexyl-8-methyl-1,4,7,10,13,16,19,22,25,28-decaoxodotriacontahydropyrrolo[1,2-a][1,4,7,10,13,16,19,22,25,28]decaazacyclotriacontin-12-yl)methyl)phenyl)-3,12-dioxo-5,8-dioxa-2,11-diazatridecan-13-yl)-3,12,16-trioxo-5,8-dioxa-2,11,15-triazaheptadecan-17-yl)-1,4,7,10-tetraazacyclododecane-1,4,7-triyl)triacetic acid (Example M1, TFA salt)

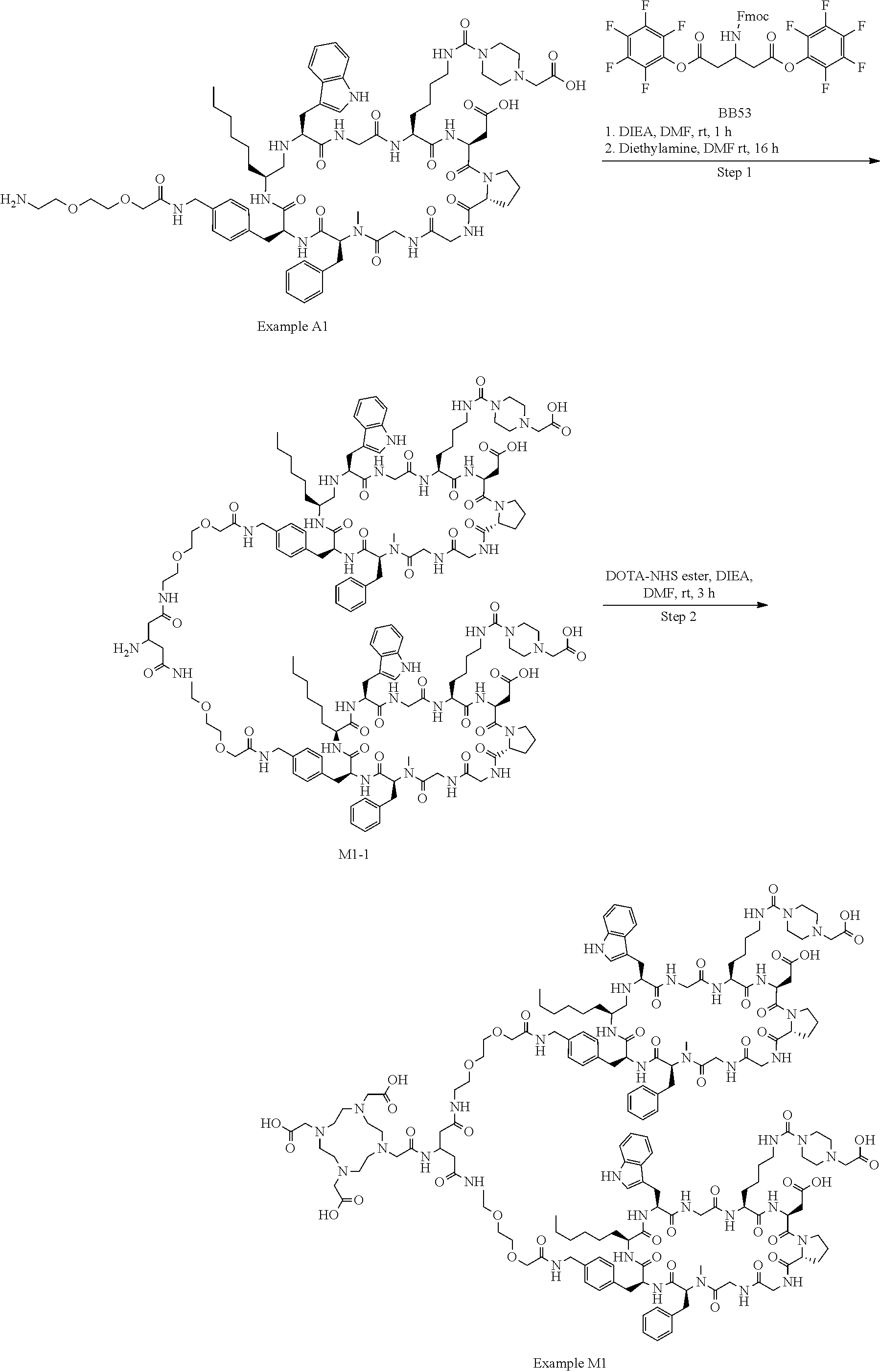

### Scheme 5.1.5. Synthesis of Example M1 (Divalent FAP Ligand)

### Step 1. M1-1

Example A1 (25.6 mg, 14.90 μmol, 2.05 eq., TFA salt) was dissolved in DMF (0.5 mL), treated with DIEA (9.7 mg, 13.0 μL, 74.89 μmol, 10.3 eq.) and the reaction mixture was stirred at rt for 5 min. Then a solution of BB53 (5.1 mg, 7.27 μmol, 1 eq.) in DMF (0.4 mL) was added. The resulting clear solution was stirred at rt for 1 h and diluted with 0.2 mL of ACN/H₂O (2/1). Then diethylamine (16.0 mg, 22.6 μL, 218.1 μmol, 30 eq.) was added and the reaction mixture was stirred at rt for 16 h. The product was isolated by preparative reversed-phase HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford M1-1 (17.5 mg, 4.7 μmol, 64% yield, TFA salt) as a white solid. LCMS method P-1, t_{R}=4.21 min, [M+2H]²⁺=1547.3.

### Step 2. Example M1

M1-1 (17.5 mg, 92% Wt, 4.7 μmol, 1.0 eq., TFA salt) was dissolved in DMF (1 mL), treated with DIEA (19.7 mg, 26.6 μL, 152.8 μmol, 32.6 eq.) and and the reaction mixture was stirred at rt for 5 min. DOTA-NHS ester (20.5 mg, 5.75 eq., 27.0 mol, trifluoroacetate-hexafluorophosphate) was added and the reaction mixture was stirred at rt for 3 h. The product was isolated by preparative reversed-phase HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example M1 (7.0 mg, 1.8 μmol, 38% yield) as a white solid. LCMS method P-1; t_{R}=4.11 min, [M+2H]²⁺=1740.4.

Example M1 was labeled with natural lutetium in analogy to Example C1:

| Example | | |
|---|---|---|
| No. | Description | LCMS |
| M2 | Example M1 labelled | Method P-1, t_{R} = 4.34 min, |
| | with Lu | [M + 2H]²⁺ = 1826.3 |

Example M3 was synthesized in analogy to the Example M1 with DOTA coupling and lutetium labeling performed in one pot in analogy to Example C21

| Example | | |
|---|---|---|
| No. | Structure/Example Name | LCMS |
| M3 | 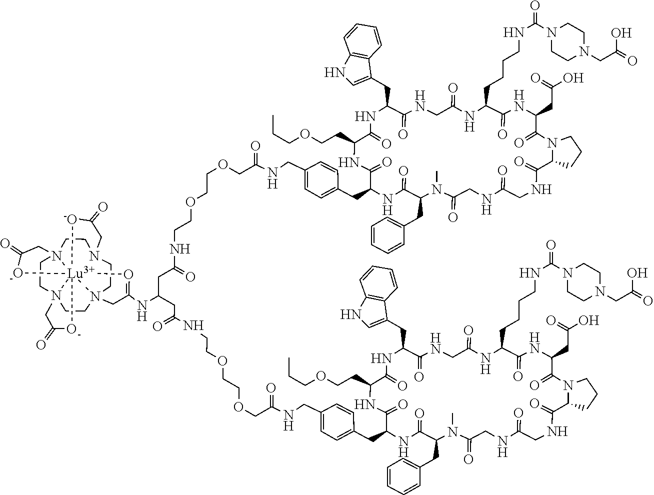 2,2′,2″-(10-(1-(4-(((9S,12S,15S,18S,24S,27S,32aR)-18-((1H-indol-3-yl)methyl)-9- benzyl-27-(carboxymethyl)-24-(4-(4-(carboxymethyl)piperazine-1- carboxamido)butyl)-8-methyl-1,4,7,10,13,16,19,22,25,28-decaoxo-15-(2- propoxyethyl)dotriacontahydropyrrolo[1,2- a][1,4,7,10,13,16,19,22,25,28]decaazacyclotriacontin-12-yl)methyl)phenyl)-14-(1-(4- (((9S,12S,15S,18S,24S,27S,32aR)-18-((1H-indol-3-yl)methyl)-9-benzyl-27- (carboxymethyl)-24-(4-(4-(carboxymethyl)piperazine-1-carboxamido)butyl)-8- methyl-1,4,7,10,13,16,19,22,25,28-decaoxo-15-(2- propoxyethyl)dotriacontahydropyrrolo[1,2- a][1,4,7,10,13,16,19,22,25,28]decaazacyclotriacontin-12-yl)methyl)phenyl)-3,12- dioxo-5,8-dioxa-2,11-diazatridecan-13-yl)-3,12,16-trioxo-5,8-dioxa-2,11,15- triazaheptadecan-17-yl)-1,4,7,10-tetraazacyclododecane-1,4,7-triyl)triacetic acid labeled with Lu | Method P-1 t_{R} = 3.80 min [M + 2H]²⁺ = 1828.3 |

### 5.1.5 Synthesis of 2,2′,2″-(10-(1-(4-(((3S,9S,12S,15S,18S,24S,27S,32aR)-18-((1H-indol-3-yl)methyl)-9-benzyl-27-(carboxymethyl)-3-(4-(4-(carboxymethyl)piperazine-1-carboxamido)butyl)-24-(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-12-yl)methyl)phenoxy)-18-(17-(4-(((3S,9S,12S,15S,18S,24S,27S,32aR)-18-((1H-indol-3-yl)methyl)-9-benzyl-27-(carboxymethyl)-3-(4-(4-(carboxymethyl)piperazine-1-carboxamido)butyl)-24-(4-hydroxybenzyl)-8-methyl-1,4,7,10,13,16,19,22,25,28-decaoxo-15-pentyldotriacontahydropyrrolo[1,2-a][1,4,7,10,13,16,19,22,25,28]decaazacyclotriacontin-12-yl)methyl)phenoxy)-2,14-dioxo-6,9,12-trioxa-3,15-diazaheptadecyl)-4,16,20-trioxo-6,9,12-trioxa-3,15,19-triazahenicosan-21-yl)-1,4,7,10-tetraazacyclododecane-1,4,7-triyl)triacetic acid (Example M4, TFA salt)

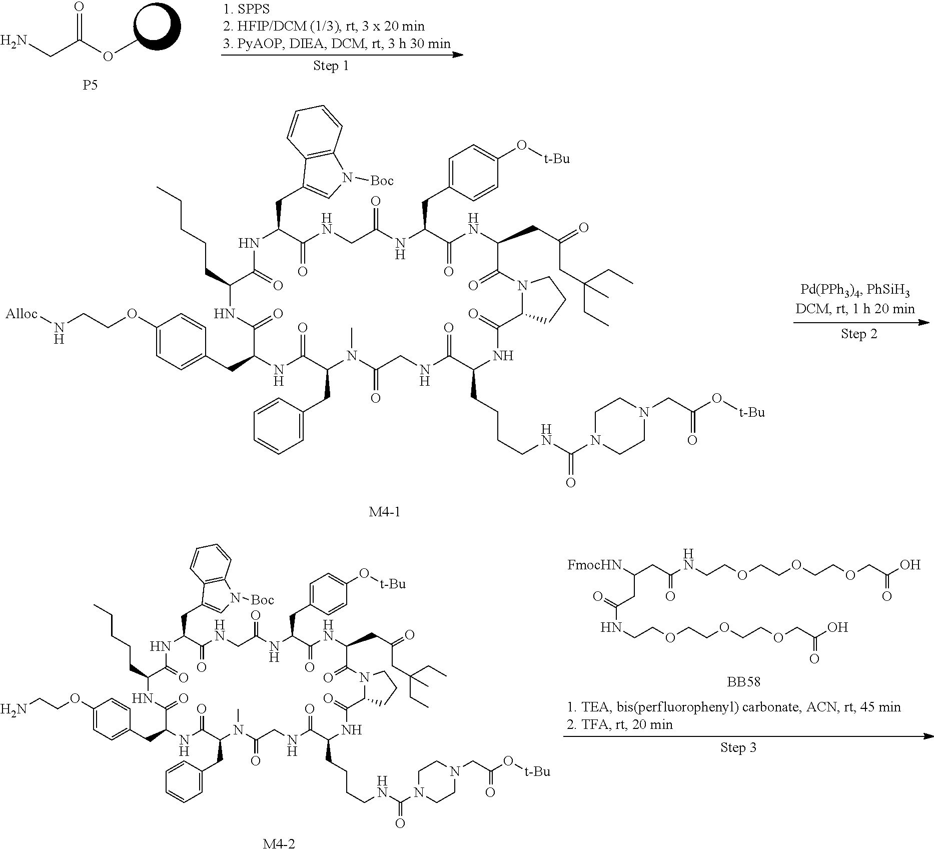

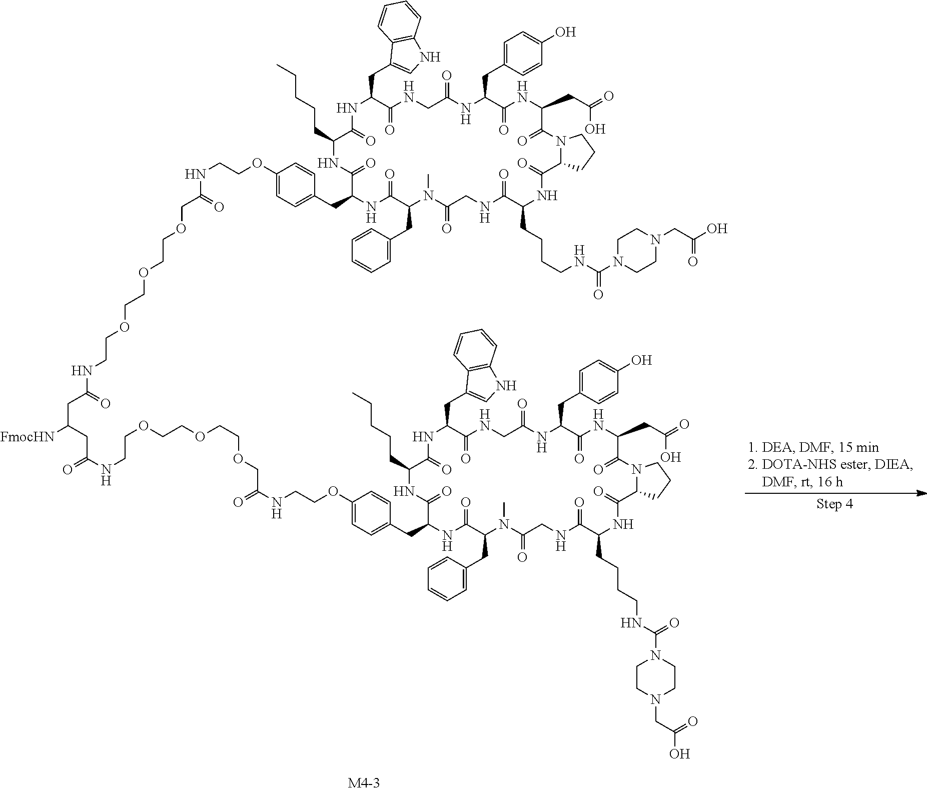

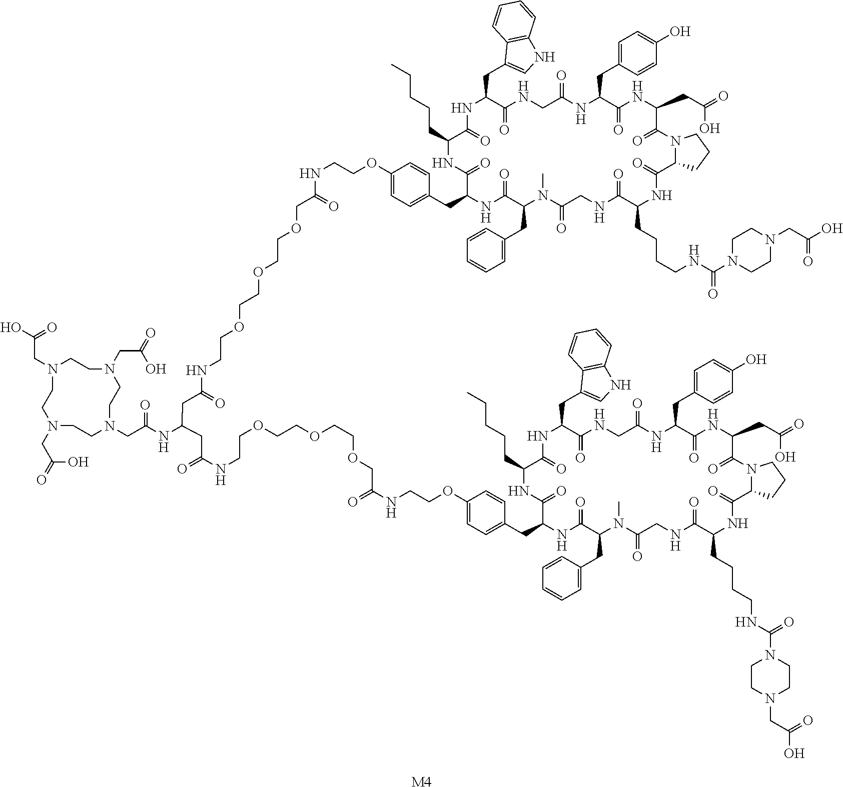

### Scheme 5.1.5. Synthesis of Example M4 (Divalent FAP Ligand)

### Step 1. M4-1

Step 1-1: SPPS was done on the CEM Liberty Prime Synthesizer using commercial preloaded resin P1 (633 mg, 0.5 mmol, 0.79 mmol/g) (corresponding to A²-resin). All amino acid couplings were performed twice following the same method: After automated addition of Fmoc-amino acid (0.25 or 0.5 M in DMF, 2 or 4 eq., see Table 5.4), Oxyma Pure®+DIEA (0.25 M Oxyma Pure® and 0.1 M DIEA in DMF, 4 eq. of Oxyma Pure®, 1.6 eq. of DIEA), and DIC (4 M in DMF, 8 eq.) the reaction mixture was heated at 50° C. for 20 min. Fmoc removal was performed twice, using pyrrolidine/DMF (1/3) (22.8 eq), automatically added to the previous amino acid coupling reaction mixture at rt for 10 min. The amino acids used are summarized in Table 5.4.

TABLE 5.4

| | Amino | | | Amino |
|---|---|---|---|---|
| Synthesis | acid | | Con- | acid, |
| cycle | residue* | Amino acid | centration | equivalents |
| 1 | A¹ | BB41 | 0.25M | 2 eq. |
| 2 | A¹⁰ | Fmoc-D-Pro-OH | 0.25M | 2 eq. |
| 3 | A⁹ | Fmoc-Asp(OMpe)-OH | 0.25M | 2 eq. |
| 4 | A⁸ | Fmoc-Tyr(tBu)-OH | 0.5M | 4 eq. |
| 5 | A⁷ | Fmoc-Gly-OH | 0.5M | 4 eq. |
| 6 | A⁶ | Fmoc-Trp(Boc)-OH | 0.5M | 4 eq. |
| 7 | A⁵ | Fmoc-Ahp-OH | 0.25M | 2 eq. |
| 8 | A⁴ | BB59 | 0.25M | 2 eq. |
| 9 | A³ | Fmoc-NMePhe-OH | 0.5M | 4 eq. |

*Refers to the corresponding amino acid residue of cyclic peptide, P, as found, e.g., in Formulae (I), (Ia), (Ib), (Ic), (Id), (I-i), or in the Table of Example III.

Step 1-2: To the resin from Step 1-1 was added HFIP/DCM (1/3) (4 mL). The resin was shaken at rt for 20 min. The cleavage solution was filtered off and the procedure was repeated (2×). The resin was washed with HFIP/DCM (1/3) (4 mL). The combined cleavage and washing solutions were concentrated to dryness in vacuo. The residue was redissolved in tert-BuOH (4 mL) and H₂O (4 mL) and lyophilized.

Step 1-3: The residue from Step 1-2 was dissolved in DCM (200 mL) and treated with DIEA (418.8 mg, 564.0 μL, 3.24 mmol, 15.0 eq.) and PyAOP (282.0 mg, 0.54 mmol, 2.5 eq.). The resulting solution was stirred at rt for 3 h 30 min, then concentrated to dryness in vacuo. The crude was dissolved in 20 mL of ACN/H₂O (2/1) and lyophilized. The product was isolated by preparative reversed-phase HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford M4-1 (189.0 mg, 91.5 μmol, 18% yield, TFA salt) as a white solid. LCMS method P-1, t_{R}=6.80 min, [M+H]⁺=1849.0.

### Step 2. M4-2

M4-1 (189.0 mg, 95% Wt, 91.5 μmol, TFA salt) was dissolved in DCM (5.0 mL), treated with Phenylsilane (165.9 mg, 191.0 μL, 1.53 mmol, 16.8 eq.) and the reaction mixture was stirred at rt for 5 min. Pd(PPh₃)₄, (12.0 mg, 10.38 μmol, 0.11 eq.) was added and the reaction mixture was stirred at rt for 1 h 20 min, then concentrated to dryness in vacuo. The crude was isolated by preparative reversed-phase HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford M4-2 (113.0 mg, 54.0 μmol, 59% yield, TFA salt) as a white solid. LCMS method P-1, t_{R}=5.84 min, [M+H]⁺=1765.0.

### Step 3. M4-3

Step 3-1: BB58 (18.6 mg, 24.87 μmol, 1.0 eq.) was suspended in ACN (3.0 mL) and treated with TEA (75.5 mg, 104.0 μL, 746.2 μmol, 30.0 eq.), followed by addition of bis(perfluorophenyl) carbonate (33.3 mg, 84.57 mmol, 3.4 eq.). The reaction mixture was stirred at rt for 15 min. Then a solution of M4-2 (106.0 mg, 95% Wt, 50.52 μmol, 2.0 eq., TFA salt) in ACN (1.0 mL) was added. The resulting clear solution was stirred at rt for 30 min, then concentrated to dryness in vacuo.

Step 3-2: The residue was then dissolved in TFA (5.0 mL) and stirred at rt for 30 min, then concentrated to dryness in vacuo and the product was isolated by preparative reversed-phase HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford M4-3 (53.0 mg, 13.67 μmol, 55% yield, TFA salt) as a white solid. LCMS method P-3, t_{R}=2.43 min, [M+2H]²+=1824.4.

### Step 4. Example M4

Step 4-1: M4-3 (53.0 mg, 13.67 μmol, 1.0 eq., TFA salt) was dissolved in DMF (1.5 mL) and treated with diethylamine (20.0 mg, 28.3 μL, 273.4 μmol, 20 eq.). The resulting solution was stirred at rt for 15 min, then concentrated to dryness in vacuo. The crude was dissolved in 3.0 mL of ACN/H₂O (2/1) and lyophilized.

Step 4-2: The residue from Step 4-1 was dissolved in DMF (1.5 mL), treated with DIEA (22.3 mg, 30.0 μL, 172.2 μmol, 12.6 eq.) and the reaction mixture was stirred at rt for 5 min. DOTA-NHS ester (40.2 mg, 3.87 eq., 52.9 mol, trifluoroacetate-hexafluorophosphate) was added and the reaction mixture was stirred at rt for 16 h. The product was isolated by preparative reversed-phase HPLC (Column: Xselect Peptide CSH C18 OBD Prep Column, 130 Å, 5 μm, 30 mm×250 mm; 30 mL/min; Eluent A: H₂O+0.1% TFA and eluent B: ACN). Pure fractions were combined and lyophilized to afford Example M4 (24.5 mg, 5.8 μmol, 42% yield, TFA salt) as a white solid. LCMS method P-1; t_{R}=4.17 min, [M+2H]²⁺=1906.4.

Example M5 was labeled with natural lutetium in analogy to Example C1:

| Example | | |
|---|---|---|
| No. | Description | LCMS |
| M5 | Example M4 labelled | Method P-1, t_{R} = 4.37 min, |
| | with Lu | [M + 2H]²⁺ = 1992.2 |

### 6 Radiolabeling

### 6.1 General Procedures

The compounds of the present invention can be radiolabeled using a variety of methods, which lead to high radiochemical yields and purities of the final products. The following methods represent a selection of possible radiolabeling methods suitable for the compounds disclosed herein (specifically the chelator-peptide conjugates, e.g., DOTA-peptide). Other variations of the radiolabeling processes (e.g., different salts of the precursors, different labeling buffers, reaction pH's, temperatures, radiometals) are also valid and should result in similar intermediate and final products.

For radiolabeling with [¹⁷⁷Lu]LuCl₃, [¹⁷⁷Lu]LuCl₃ in 0.04 M HCl, as received from the supplier (50-1220 MBq), was diluted in labeling buffer (a combination of, e.g., acetic acid/gentisic acid /ascorbic acid/sodium acetate) at a preferable pH from 4.5 to 5.5. A solution containing the DOTA-peptide (“B” Examples) (1.0-150 nmol in a minimum volume) was subsequently added. The reaction volume was then corrected to 100-1000 μL with labeling buffer. The reaction mixture was heated for 5-15 min at 95° C. and then left to reach room temperature. Finally, the product was diluted in formulation solution to reach the desired activity concentration (27-782 MBq/mL). The details of each radiolabeling method with [¹⁷⁷Lu]LuCl₃ are specified in Section 6.3.

For radiolabeling with [⁶⁸Ga]GaCl₃, [⁶⁸Ga]GaCl₃ in HCl 0.1 M (870-905 MBq) was obtained from a ⁶⁸Ge/⁶⁸Ga generator and eluted directly into a glass vial containing an aqueous solution of gentisic acid (0.52-0.75 mg). The reaction pH was adjusted to approximately 3.7 with sodium formate. A solution containing the DOTA-peptide (“B” Examples) (30-150 nmol in a minimum volume) was subsequently added to reach the desired activity concentration (5-33 MBq/mL). The reaction mixture was heated for 10-14 min at 95° C. and then left to reach room temperature. The details of each radiolabeling method with [⁶⁸Ga]GaCl₃ are specified in Section 6.4.

Synthesis of [¹⁸F]-labeled peptides is described in Section 6.5. Synthesis of [22′Ac]Ac-labeled peptides is described in Section 6.6.

### 6.2 Analytical Methods

Radio-HPLC Analytical Method A: Eluent A: Water+0.1% TFA; Eluent B: ACN+0.1% TFA; Column temperature: 30° C.; Flow: 1.5 mL/min; Column: XBridge C18, 3.5 μm, 4.6×50 mm; Gradient: from 5 to 95% B in 7.0 min, from 95 to 5% B in 1.5 min, hold 5% B for 2.5 min.

Radio-HPLC Analytical Method B: Eluent A: Water+0.1% TFA; Eluent B: ACN+0.1% TFA; Column temperature: 60° C.; Flow: 0.5 mL/min; Column: AdvanceBio Peptide Plus 4.6×150 mm, 2.7 μm; Gradient: from 19.3 to 24% B in 6.0 min, hold 24% B for 6 min, from 24 to 33.5% B in 13.0 min, from 33.5 to 95% B in 0.1 min, from 95 to 95.2% B in 2.9 min, from 95.2 to 19.3% B in 0.1 min, hold 19.3% B for 6.9 min.
Radio-HPLC Analytical Method C: Eluent A: Water+0.1% TFA; Eluent B: ACN+0.1% TFA; Column temperature: 25° C.; Flow: 1.0 mL/min; Column: Luna 5 m C18(2) 100 Å, LC Column 150×4.6 mm; Gradient: hold 10% B for 1 min, from 10 to 80% B in 8 min, hold 80% B for 2 min, from 80 to 10% B in 1 min.
Radio-HPLC Analytical Method D: Eluent A: Water+0.1% TFA; Eluent B: ACN+0.1% TFA; Column temperature: 25° C.; Flow: 1.0 mL/min; Column: Luna 5 m C18(2) 100 Å, LC Column 150×4.6 mm; Gradient: hold 26% B for 13.5 min, from 26 to 70% B in 1 min, hold 70% B for 0.5 min, from 70 to 26% B in 0.5 min, hold 26% B for 2.5 min.
Radio-HPLC Analytical Method E: Eluent A: Water+0.10% TFA; Eluent B: ACN+0.10% TFA; Column temperature: 60° C.; Flow: 1.0 mL/min; Column: Kinetex 2.6 μm Phenyl-Hexyl 100 Å, LC Column 100×4.6 mm; Gradient: hold 27% B for 11.9 min, from 27 to 80% B in 0.1 min, hold 80% B for 0.5 min, from 80 to 27% B in 0.5 min.
Radio-HPLC Analytical Method F: Eluent A: Water+0.10% TFA; Eluent B: ACN+0.10% TFA; Column temperature: r.t.; Flow: 1.5 mL/min; Column: Waters XSelect peptide CSH C18, 130 Å, 5 μm, 4.6×150 mm; Gradient: from 5 to 20% B in 1 min, from 20 to 50% B in 7 min, from 50 to 95% B in 1 min, hold 95% B for 0.5 min, from 95 to 5% B in 1 min
Radio-iTLC Analytical Method: Performed on glass microfiber chromatography paper impregnated with silica gel (Agilent iTLC-SG SGI0001) and using sodium citrate 0.1M (pH 4.6) as mobile phase. Rf([¹⁷⁷Lu]Lu-labeled peptides)=0.0-0.5 and Rf(unchelated ¹⁷⁷Lu³⁺)=0.8-1.0.

### 6.3 Radiolabeling with [¹⁷⁷Lu]LuCl₃

### 6.3.1 Method 1

### Materials

| Name | Supplier |
|---|---|
| Ethanol absolute ≥99.9% for analysis | Merck |
| EMSURE ® ACS, ISO, Reag. Ph | |
| Eur | |
| Gentisic acid (2,5-dihydroxybenzoic acid) 98% | Sigma-Aldrich |
| Hydrochloric acid 30% for inorganic trace analysis | Merck |
| Non-carrier added [¹⁷⁷Lu]LuCl₃ in 0.04M | ITM Medical |
| hydrochloric acid solution | Isotopes GmbH |
| Sodium acetate trihydrate for analysis | Merck |
| EMSURE ® ACS, ISO, Reag. Ph | |
| Eur | |
| Sodium chloride 0.9% solution for injectables | B. Braun |
| Sodium hydroxide 10 mol/L (10N) in | VWR |
| aqueous solution, Reagent Grade | |
| Water TraceSELECT ™, for trace analysis | Honeywell |

Labeling buffer: The labeling buffer consists of 5 μL gentisic acid solution (31.4 mg/mL), 5 μL sodium acetate solution (47.0 mg/mL), and 140 μL TraceSELECT™ water, adjusted to pH 5.5 with HCl 30%. The labeling buffer was stored at room temperature.

Formulation solution (5% EtOH in saline): The formulation solution was prepared by mixing 2.5 mL ethanol in 47.5 mL NaCl 0.9%. The formulation solution was stored at room temperature.

Peptide stock solution for radiolabeling (1 mM): The DOTA precursors (“B” Examples) were dissolved in 1.0 mL 5% ethanol in TraceSELECT™ water. The solution was fractionated and stored in a freezer at −20° C.

Radiolabeling (4-74 MBq/nmol): In a low protein binding tube, [¹⁷⁷Lu]LuCl₃ in 0.04 M HCl (50-670 MBq) was diluted in labeling buffer up to a volume of 100-300 μL, mixed, and briefly heated at 95° C. in a shielded Thermoshaker Incubator. After that, 1.0-25.5 nmol of the DOTA-peptide stock solution (1 mM) was added to the reaction mixture to fulfill a molar activity between 4-74 MBq/nmol. Ethanol was added to the reaction mixture up to a final concentration of 5%. This reaction mixture, with a pH between 4.0 and 4.5, was left to stir (600 rpm) at 95° C. for 15 min. After cooling at room temperature, the completion of the reaction was confirmed by radio-iTLC and radio-HPLC and there was no need for further purification steps. The final product was diluted with formulation solution to an activity concentration of 27-740 MBq/mL and this formulated solution was used for further stability evaluation or biological experiments.

### 6.3.2 Method 2

### Materials

| Name | Supplier |
|---|---|
| Water for analysis EMSURE ® | Merck |
| SCHOTT Type I plus | SCHOTT Pharma |
| Ascorbic acid ≥99.9998% (metals basis), | Honeywell |
| TraceSELECT™, Fluka ™ | |
| (+)-Sodium L-ascorbate BioXtra, ≥99.0% (NT) | Sigma Aldrich |
| Acetic acid (glacial) 100% anhydrous | Merck |
| for analysis EMSURE ® ACS | |
| Sodium acetate anhydrous, | Sigma Aldrich |
| for molecular biology, ≥99% | |
| Gentisic acid (2,5-dihydroxybenzoic acid), 98% | Sigma-Aldrich |
| Hydrochloric acid 30% for inorganic | Merck |
| trace analysis | |
| Non-carrier added [¹⁷⁷Lu]LuCl₃ in 0.04M | ITM Medical Isotopes |
| hydrochloric acid solution | GmbH |
| D-Mannitol, ≥98% | Sigma Aldrich |
| Sodium hydroxide >98.0%, pellets, | Merck |
| EMPROVE ® bio Ph. Eur., BP, JP, | |
| NF for biopharmaceutical production | |

Labeling buffer: The labeling buffer consists of 147.7 mg of sodium acetate and 124.8 μL acetic acid dissolved in 100 mL Milli-Q water (pH=4.7). The labeling buffer was stored at room temperature.

Sodium gentisate solution (5 mg/mL): Gentisic acid (5 mg) was dissolved in 1 mL labeling buffer. The solution was stored at 4-8° C.

Sodium gentisate solution (35 mg/mL): Gentisic acid (350 mg) and NaOH (91 mg) were dissolved in 10 mL Milli-Q water. The solution was stored at 4-8° C.

Sodium ascorbate solution (666.7 mg/mL): Sodium ascorbate was dissolved in labeling buffer. The solution was stored at 4-8° C.

Formulation solution: The formulation solution was prepared by mixing 283.8 μL sodium gentisate solution (35 mg/mL) and 225 μL sodium ascorbate solution (666.7 mg/mL).

DOTA-peptide stock solution for radiolabeling (2.0 mg/mL): The DOTA precursors (“B” Examples) were dissolved in EMSURE® water. The solution was stored at 4-8° C.

Radiolabeling (2.7-10.9 MBq/nmol): In a Schott Vial, 70 μL sodium gentisate solution (5 mg/mL),_75-150 nmol of the DOTA-peptide stock solution (2.0 mg/mL), and [¹⁷⁷Lu]LuCl₃ in 0.04 M HCl (395-1220 MBq) were diluted with labeling buffer up to 1 mL. The vial was crimped and heated at 95° C. for 5 min. After cooling the vial at room temperature for 5 min, the final product was diluted with formulation solution to an activity concentration of 257-782 MBq/mL and this formulated solution was used for further stability evaluation.

### 6.3.3 Method 3

### Materials

| Name | Supplier |
|---|---|
| Acetic acid ≥99.99% (trace metals basis) | Honeywell |
| Ascorbic acid ≥99.9998% (metals basis), | Honeywell |
| TraceSELECT ™, Fluka ™ | |
| Ethanol absolute ≥99.9% for analysis | Merck |
| EMSURE ® ACS, ISO, Reag. Ph | |
| Eur | |
| Non-carrier added [¹⁷⁷Lu]LuCl₃ in 0.04M | ITM Medical |
| hydrochloric acid solution | Isotopes |
| | GmbH |
| Phosphate Buffered Saline (PBS), pH 7,4 | VWR |
| Sodium acetate trihydrate for analysis | Merck |
| EMSURE ® ACS, ISO, Reag. Ph | |
| Eur | |
| Water TraceSELECT ™, for trace analysis | Honeywell |
| Acetic acid ≥99.99% (trace metals basis) | Honeywell |
| Ascorbic acid ≥99.9998% (metals basis), | Honeywell |
| TraceSELECT ™, Fluka ™ | |
| Ethanol absolute ≥99.9% for analysis | Merck |
| EMSURE ® ACS, ISO, Reag. Ph | |
| Eur | |
| Non-carrier added [¹⁷⁷Lu]LuCl₃ in 0.04M | ITM Medical |
| hydrochloric acid solution | Isotopes |
| | GmbH |
| Phosphate Buffered Saline (PBS), pH 7,4 | VWR |
| Sodium acetate trihydrate for analysis | Merck |
| EMSURE ® ACS, ISO, Reag. Ph | |
| Eur | |
| Water TraceSELECT ™, for trace analysis | Honeywell |

Sodium acetate solution (1.5 M): Sodium acetate trihydrate (204.0 mg) was dissolved in 1 mL TraceSELECT™ water. The solution was stored in a refrigerator at 4° C.

Ascorbic acid solution (0.04 M): Ascorbic acid (7.1 mg) was dissolved in 1 mL TraceSELECT™ water. The solution was stored in a refrigerator at 4° C.

DOTA-peptide stock solution for radiolabeling (2 mM): The DOTA precursors (“B” Examples) were dissolved in 1.0 mL TraceSELECT™ water. The solution was fractionated and stored in a freezer at −20° C.

Radiolabeling (37 MBq/nmol): In a low protein binding tube containing 35 μL sodium acetate solution (1.5 M), 35 μL ascorbic acid solution (0.04 M), 1.5 μL acetic acid, and 58 μL ethanol, was added 250 μL [¹⁷⁷Lu]LuCl₃ in 0.04 M HCl (55-530 MBq). After that, 1.5-13.8 nmol of the DOTA-peptide stock solution (2 mM) was added to the reaction mixture to fulfill a molar activity of approximately 37 MBq/nmol. This reaction mixture, with a pH of 4.7, was left to stir (450 rpm) at 95° C. for 15 min in a shielded Thermoshaker Incubator. After cooling at room temperature, the reaction mixture was diluted with PBS pH 7.4 to keep the final formulation with less than 9% of ethanol and an activity concentration of approximately 370 MBq/mL. This solution was used for further extended stability studies overtime and biological assays.

The following examples (for structures see Table 6.1) were synthesized following one the of the standard methods above:

Example G1 was synthesized from Example B23.

Example G2 was synthesized from Example B20.

Example G3 was synthesized from Example B2.

Example G4 was synthesized from Example B1.

TABLE 6.1
Examples of [¹⁷⁷Lu]Lu-labeled peptides synthesized following the general ¹⁷⁷Lu-
radiolabeling methods

| Example | | |
|---|---|---|
| No. | Structure/Example Name* | HPLC |
| G1 | 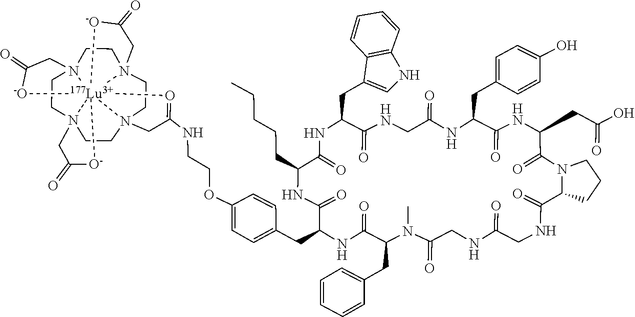 | Method A t_{R} = 3.68 min |
| | [2,2′,2″-{10-[2-({2-[4-({(9S,12S,15S,18S,24S,27S,32aR)-9-Benzyl-27- (carboxymethyl)-24-[(4-hydroxyphenyl)methyl]-18-[(1H-indol-3-yl)methyl]-8- methyl-1,4,7,10,13,16,19,22,25,28-decaoxo-15-pentyldotriacontahydropyrrolo[1,2- a][1,4,7,10,13,16,19,22,25,28]decaazacyclotriacontin-12- yl}methyl)phenoxy]ethyl}amino)-2-(oxo-κO)ethyl]-1,4,7,10-tetraazacyclododecane- 1,4,7-triyl-κ⁴N¹,N⁴,N⁷,N¹⁰}tri(acetato-κO)(3-)](¹⁷⁷Lu)lutetium | |
| G2 | 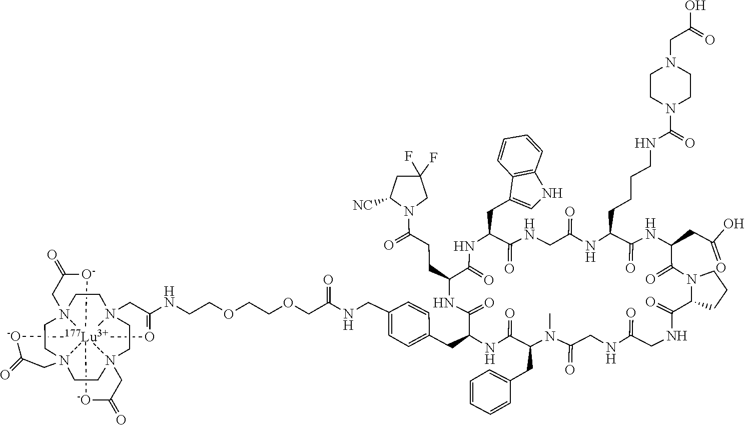 | Method A t_{R} = 3.21 min |
| | (Cyclo{_{L}-α-aspartyl-_{D}-prolylglycylglycyl-N-methyl-_{L}-phenylalanyl-4-[3-oxo-12-(oxo- κO)-13-{4,7,10-tris[(carboxy-κO)methyl]-1,4,7,10-tetraazacyclododecan-1-yl- κ⁴N¹,N⁴,N⁷,N¹⁰}-5,8-dioxa-2,11-diazatridecan-1-yl]-_{L}-phenylalanyl-5-(2-cyano-4,4- difluoropyrrolidin-1-yl)-5-oxo-_{L}-norvalyl-_{L}-tryptophylglycyl-N⁶-[4- (carboxymethyl)piperazine-1-carbonyl]-_{L}-lysyl})(¹⁷⁷Lu)lutetium | |
| G3 | 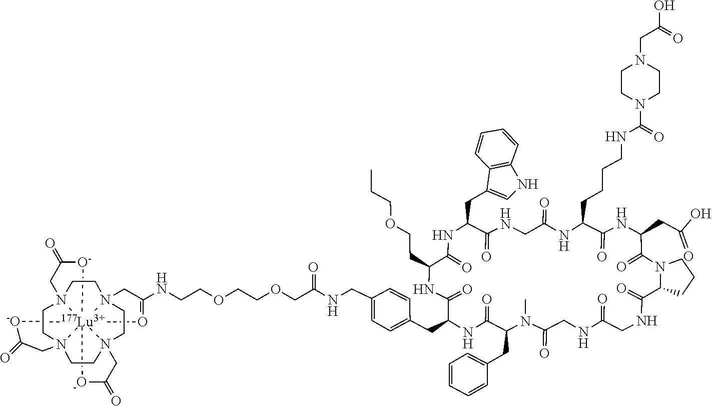 | Method A t_{R} = 3.25 min Method C t_{R} = 6.63 min |
| | (Cyclo{_{L}-α-aspartyl-_{D}-prolylglycylglycyl-N-methyl-_{L}-phenylalanyl-4-[3-oxo-12-(oxo- κO)-13-{4,7,10-tris[(carboxy-κO)methyl]-1,4,7,10-tetraazacyclododecan-1-yl- κ⁴N¹,N⁴,N⁷,N¹⁰}-5,8-dioxa-2,11-diazatridecan-1-yl]-_{L}-phenylalanyl-O-propyl-_{L}- homoseryl-_{L}-tryptophylglycyl-N⁶-[4-(carboxymethyl)piperazine-1-carbonyl]-_{L}- lysyl})(¹⁷⁷Lu)lutetium | |
| G4 | 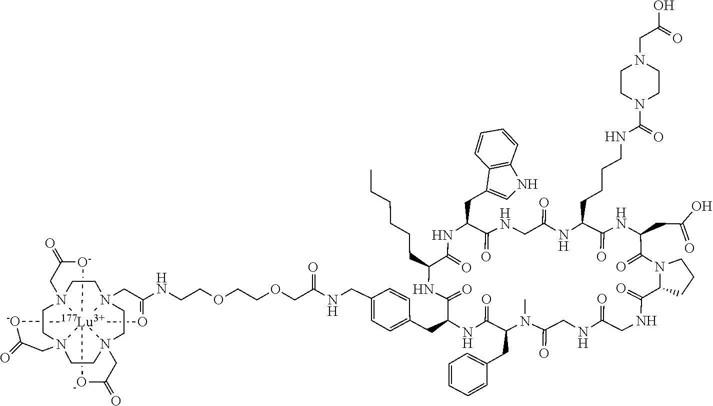 | Method A t_{R} = 3.53 min Method B t_{R} = 14.26 min Method C t_{R} = 7.12 min |

[2,2′,2″-(10-{1-[4-({(9S,12S,15S,18S,24S,27S,32aR)-9-Benzyl-27-(carboxymethyl)- 24-(4-{[4-(carboxymethyl)piperazine-1-carbonyl]amino}butyl)-15-hexyl-18-[(1H- indol-3-yl)methyl]-8-methyl-1,4,7,10,13,16,19,22,25,28- decaoxodotriacontahydropyrrolo[1,2- a][1,4,7,10,13,16,19,22,25,28]decaazacyclotriacontin-12-yl}methyl)phenyl]-3-oxo- 12-(oxo-κO)-5,8-dioxa-2,11-diazatridecan-13-yl}-1,4,7,10-tetraazacyclododecane- 1,4,7-triyl-κ⁴N¹,N⁴,N⁷,N¹⁰)tri(acetato-κO)(3-)](¹⁷⁷Lu)lutetium

* The drawings represent one way in which the [¹⁷⁷Lu]Lu-DOTA complexes (¹⁷⁷Lu radiolabeled-DOTA conjugates) could be illustrated. The same compound could be drawn, e.g., with a different number of coordination bonds, represented by either dashed or solid lines.

The initial radiochemical purity of [¹⁷⁷Lu]Lu-labeled peptides was analyzed by radio-HPLC and radio-iTLC and the stability in solution at 24° C. was evaluated 1-96 h after the end of synthesis by radio-HPLC.

TABLE 6.2
Radiochemistry results for [¹⁷⁷Lu]Lu-labeled peptides

| | Radio- | Molar activity | | | | | | | | |
|---|---|---|---|---|---|---|---|---|---|---|
| Ex. | labeling | final product | Initial | Initial | 1 | 4 | 5 | 24 | 48 | 72 |
| No. | method | (MBq/nmol) | (iTLC) | (HPLC) | (h) | (h) | (h) | (h) | (h) | (h) |
| G1 | Method 1 | 4 to 5 | 3.4 | 98.1 | 98.0 | 94.4 | | | | |
| G2 | Method 1 | 74 | 0 | 96.6 | 95.7 | 94.8 | | | | |
| G3 | Method 1 | 74 | 0 | 98.8 | 97.8 | 89.8 | | | | |
| G3 | Method 3 | 37 | 0.5 | 99.2 | | | 99.2 | 99.0 | 98.8 | |
| G4 | Method 1 | 4 | 0 | 98.1 | 97.7 | 95.4 | | | | |
| G4 | Method 2 | 3-11 | 0 | 99.7 | | | | 99.3 | 98.4 | 97.9 |
| G4 | Method 3 | 37 | 0.5 | 99.1 | | | 98.1 | 96.9 | | 95.8 |

### 6.4 Radiolabeling with [⁶⁸Ga]GaCl₃

### Materials

| Name | Supplier |
|---|---|
| Formic acid, EMSURE ® ACS for analysis, Supelco ® | Merck |
| [⁶⁸Ga]GaCl₃ in 0.1M hydrochloric acid solution | IRE Elit ⁶⁸Ge/⁶⁸Ga |
| | generator |
| Gentisic acid (2,5-dihydroxybenzoic acid) 98% | Sigma-Aldrich |
| Sodium hydroxide 98.5-100.5%, pellets, | VWR |
| AnalaR NORMAPUR ® Reag. | |
| Ph. Eur. analytical reagent | |
| Water for analysis EMSURE ® | Merck |
| Water for injection, USP | VWR |

Gentisic acid solution (2.6 mg/mL): Gentisic acid (2.6 mg) was dissolved in 1 mL of EMSURE® water. The solution was stored in a refrigerator at 4° C.

Gentisic acid solution (126.0 μg/mL): Gentisic acid (6.3 mg) was dissolved in 50 mL of EMSURE® water. The solution was stored in a refrigerator at 4° C.

DOTA-peptide stock solution for radiolabeling (2.0 mg/mL): The DOTA precursors (“B” Examples) were dissolved in EMSURE® water. The solution was stored at 4-8° C. Radiolabeling Method 4: In a glass vial containing 200 μL of gentisic acid solution (2.6 mg/mL) it was added approximately 1.1 mL of [⁶⁸Ga]GaCl₃ in 0.1 M HCl (approx. 870 MBq) directly transferred from the ⁶⁸Ge/⁶⁸Ga generator. After that, 110 μL of sodium formate and 30 nmol of the DOTA-peptide stock solution (2.0 mg/mL) were added to the reaction mixture to fulfill a molar activity of approximately 23 MBq/nmol at the end of the synthesis. This reaction mixture, with pH 3.7, was left incubating at 95° C. for 10 min in a shielded heating block. After cooling at room temperature, the radiochemical purity was followed by radio-HPLC and no further purification or formulation was performed.

Radiolabeling Method 5: In a glass vial containing 5985 μL of gentisic acid solution (126.0 μg/mL) it was added approximately 1.1 mL of [⁶⁸Ga]GaCl₃ in 0.1 M HCl (approx. 905 MBq) directly transferred from the ⁶⁸Ge/⁶⁸Ga generator. After that, 250 μL of sodium formate and 150 nmol of the DOTA-peptide stock solution (2.0 mg/mL) were added to the reaction mixture to fulfill a molar activity of approximately 5 MBq/nmol at the end of the synthesis. This reaction mixture, with pH of approximately 3.7, was left incubating at 95° C. for 14 min in a shielded heating block. After cooling at room temperature for 5 min, the radiochemical purity was followed by radio-HPLC and no further purification or formulation was performed.

The following examples (for structures see Table 6.3) were synthesized following the standard method above:

Example H1 was synthesized from Example B2.

Example H2 was synthesized from Example B1.

TABLE 6.3
Examples of [68Ga]Ga-labeled peptides synthesized following the general 68Ga-
labeling methods

| Example | | |
|---|---|---|
| No. | Structure/Example Name* | HPLC |
| H1 | 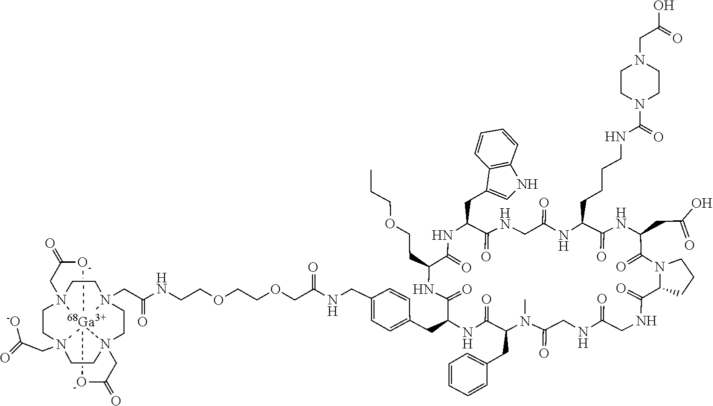 | Method D t_{R} = 11.30 min |
| | (Cyclo{_{L}-α-aspartyl-_{D}-prolylglycylglycyl-N-methyl-_{L}-phenylalanyl-4-(13-{7- (carboxymethyl)-4,10-bis[(carboxy-κO)methyl]-1,4,7,10-tetraazacyclododecan-1-yl- κ⁴N¹,N⁴,N⁷,N¹⁰}-3,12-dioxo-5,8-dioxa-2,11-diazatridecan-1-yl)-_{L}-phenylalanyl-O- propyl-_{L}-homoseryl-_{L}-tryptophylglycyl-N⁶-[4-(carboxymethyl)piperazine-1- carbonyl]-_{L}-lysyl})(⁶⁸Ga)gallium | |
| H2 | 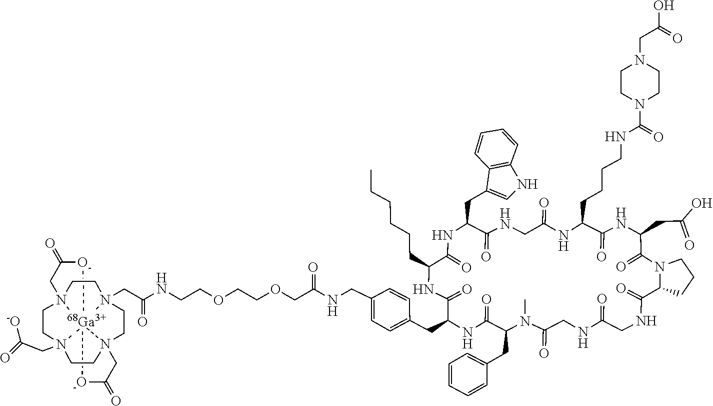 | Method E t_{R} = 8.60 min |

[2,2′,2″-(10-{1-[4-({(9S,12S,15S,18S,24S,27S,32aR)-9-Benzyl-27-(carboxymethyl)- 24-(4-{[4-(carboxymethyl)piperazine-1-carbonyl]amino}butyl)-15-hexyl-18-[(1H- indol-3-yl)methyl]-8-methyl-1,4,7,10,13,16,19,22,25,28- decaoxodotriacontahydropyrrolo[1,2- a][1,4,7,10,13,16,19,22,25,28]decaazacyclotriacontin-12-yl}methyl)phenyl]-3,12- dioxo-5,8-dioxa-2,11-diazatridecan-13-yl}-1,4,7,10-tetraazacyclododecane-1,4,7- triyl-κ⁴N¹,N⁴,N⁷,N¹⁰)tri(acetato-κO)(3-)](⁶⁸Ga)gallium

* The drawings represent one way in which the [⁶⁸Ga]Ga-DOTA complexes (⁶⁸Ga radiolabeled-DOTA conjugates) could be illustrated. The same compound could be drawn, e.g., with a different number of coordination bonds, represented by either dashed or solid lines.

The initial radiochemical purity of [⁶⁸Ga]Ga-labeled peptides was analyzed by radio-HPLC and the stability in solution at 24° C. was evaluated 1-4 h after the end of synthesis by radio-HPLC.

TABLE 6.4
Radiochemistry results for [⁶⁸Ga]Ga-labeled peptides

| Example | Radiolabeling | Molar activity final | | |
|---|---|---|---|---|
| No. | method | product (MBq/nmol) | Initial | 4 h |
| H1 | Method 4 | 33 | 96.0 | 95 |
| H2 | Method 5 | 5 | 95.7 | 93 |

### 6.5 Radiosynthesis of [¹⁸F]-Labeled Peptide I1

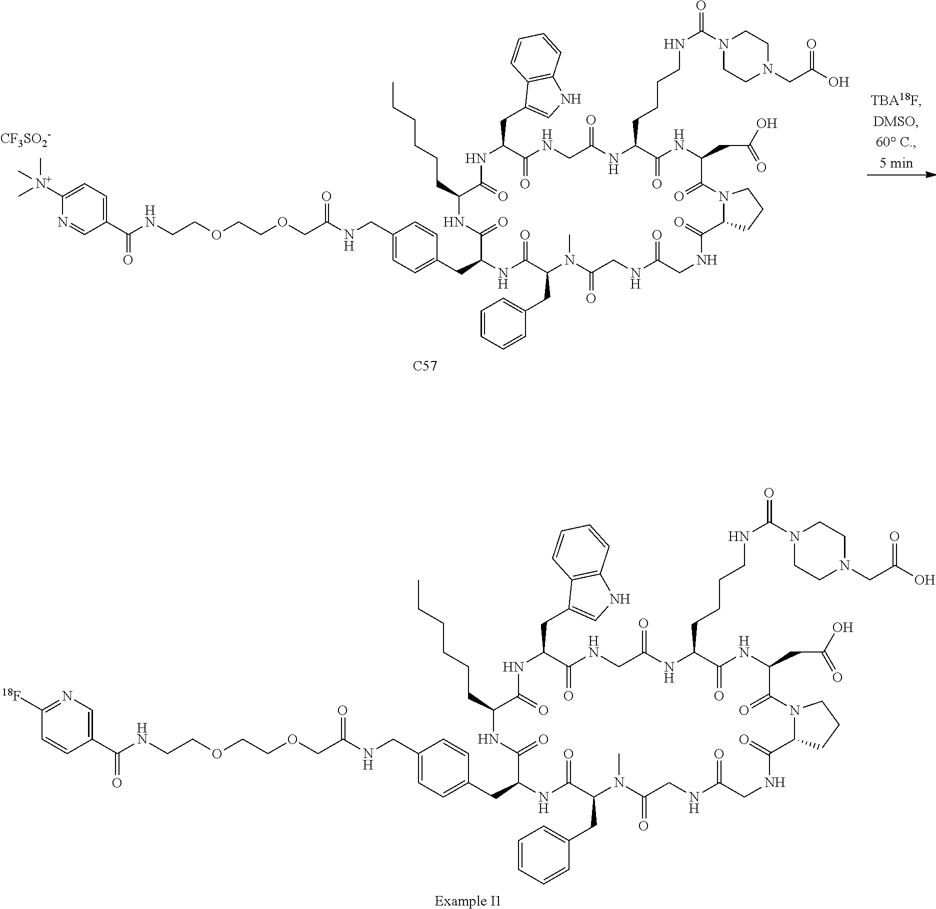

### Scheme 6.5. Synthesis of [¹⁸F]-Labeled Peptide I1

[¹⁸F]fluoride was loaded onto Chromafix PS-HCO₃ cartridge and eluted with the elution cocktail (5 mg of TBAB in 400 μL of ACN and 100 μL of water). The mixture was dried azeotropically using a steam of nitrogen at 100° C. for 10 min. Another 800 μL of anhydrous ACN were added and the mixture was dried again for another 5 min. The drying cycle was repeated once and the temperature was reduced to 60° C. Then the TMAP precursor C57 (1 mg in 100 μL of anhydrous DMSO) was added. The reaction mixture was heated at 60° C. for 5 min, then diluted with 100 μL of water and the product was purified by a semi-preparative HPLC. The initial radiochemical purity was analyzed by radio-HPLC and the stability in solution at 24° C. was evaluated 1-4 h after the end of synthesis by radio-HPLC.

### 6.6 Radiosynthesis of [²²⁵Ac]Ac-Labeled Peptide JI

### Materials

| Name | Supplier |
|---|---|
| Ethanol absolute ≥99.9% for analysis | Merck |
| EMSURE ® ACS, ISO, Reag. Ph | |
| Eur | |
| Hydrochloric acid 30% for inorganic trace analysis | Sigma Aldrich |
| L-ascorbic acid, BioXtra, >99.0% | Sigma Aldrich |
| DTPA | Sigma Aldrich |
| Ac-225 | ITM |
| Water TraceSELECT ™, for trace analysis | VWR |

Radiolabeling buffer: 0.5 M L-ascorbic acid. The labeling buffer was stored at room temperature.

Formulation buffer: 25 mg/mL sodium ascorbate with 0.22 mg/mL DTPA. The formulation solution was stored at room temperature.

Peptide stock solution for radiolabeling (1 mg/mL): The DOTA precursors (“B” Examples) were dissolved in 5% ethanol in TraceSELECT™ water.

Ac-225 was dissolved in 0.04 M HCl to achieve an activity concentration of 50 MBq/mL and mixed with radiolabeling buffer in a ratio 1:1. pH was adjusted to 4.5-5. Peptide stock solution was added to reach the desired specific activity. The reaction mixture was stirred (600 rpm) at 95° C. for 15 min. After cooling to room temperature, the completion of the reaction was confirmed by radio-iTLC and radio-HPLC and there was no need for further purification steps. For radio-TLC, the plate was cut in half and each half immediately measured twice by gamma counting. The Ac-225 activity was estimated based on the Fr-221 and Bi-213 peak in the spectrum. The final product was diluted in formulation buffer to an activity concentration of 740 kBq/mL (74 kBq/100 μL). RCP of >98% was determined by radio-HPLC.

TABLE 6.5
Examples of [225Ac]Ac-labeled peptides synthesized following the general Ac-225
radiolabeling methods

| Example | | |
|---|---|---|
| No. | Structure/Example Name* | HPLC |
| J1 | 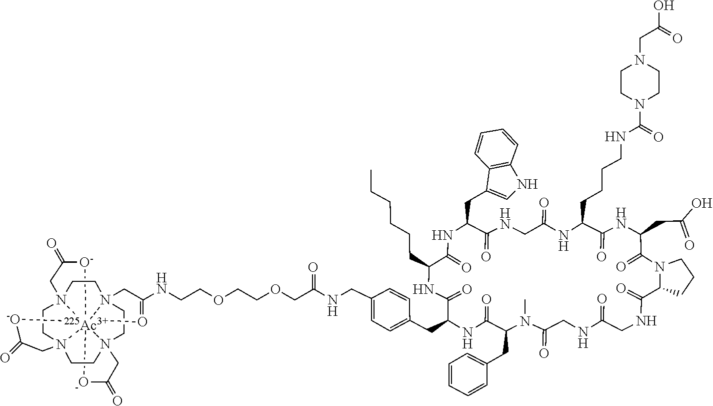 | Method F t_{R} = 6.4 min |

[2,2′,2″-(10-{1-[4-({(9S,12S,15S,18S,24S,27S,32aR)-9-Benzyl-27-(carboxymethyl)-24- (4-{[4-(carboxymethyl)piperazine-1-carbonyl]amino}butyl)-15-hexyl-18-[(1H-indol-3- yl)methyl]-8-methyl-1,4,7,10,13,16,19,22,25,28-decaoxodotriacontahydropyrrolo[1,2- a][1,4,7,10,13,16,19,22,25,28]decaazacyclotriacontin-12-yl}methyl)phenyl]-3-oxo-12- (oxo-κO)-5,8-dioxa-2,11-diazatridecan-13-yl}-1,4,7,10-tetraazacyclododecane-1,4,7- triyl-κ⁴N¹,N⁴,N⁷,N¹⁰)tri(acetato-κO)(3-)](²²⁵ Ac)actinium

* The drawings represent one way in which the [²²⁵Ac]Ac-DOTA complexes (²²⁵Ac radiolabeled-DOTA conjugates) could be illustrated. The same compound could be drawn, e.g., with a different number of coordination bonds, represented by either dashed or solid lines.

TABLE 6.6
Radiochemistry results for [²²⁵Ac]Ac-labeled peptides

| | Molar activity | | |
|---|---|---|---|
| | final product | | |
| Example No. | (kBq/nmol) | Initial | 4 h |
| J1 | 73.6 | 98.0 | 98.1 |

### 7 Building Blocks

### 7.1 Resin Preparation

### 7.1.1 Synthesis of H-Gly-2-Cl-Trt Resin (P1)

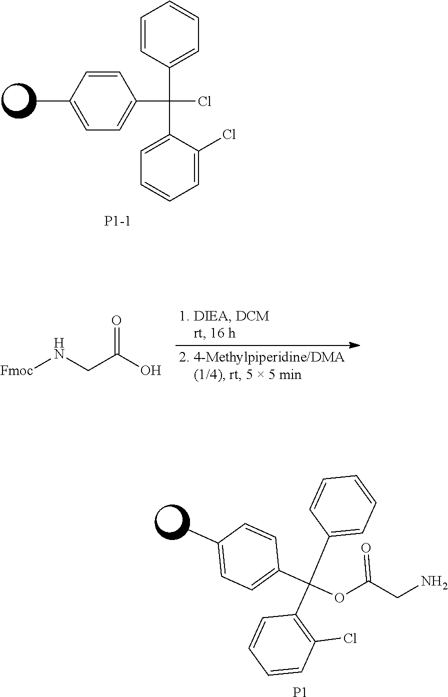

### Scheme 7.1.12. Synthesis of H-Gly-2-Cl-Trt Resin (P1)

2-Chlorotrityl chloride PS resin P1-1 (loading 1.0-1.6 mmol/g, calculated with 1.6 mmol/g) (9.375 g, 3 eq., 15.00 mmol) was washed with DCM (3×70 mL). A solution of Fmoc-Gly-OH (1.487 g, 1 eq., 5.000 mmol) in DCM (80 mL) and DIEA (3.878 g, 5.23 mL, 6 eq., 30.00 mmol) was added. The suspension was shaken at rt for 16 h. The resin was drained and then thoroughly washed sequentially with DCM/MeOH/DIPEA (17:2:1), DCM and DMA.

For Fmoc removal and determination of the loading the resin was shaken repeatedly at rt with 4-methylpiperidine/DMA (1/4) (5×5 min, each time 80 mL) and washed with DMA (2×60 mL). The combined solutions were diluted with MeOH and used for the determination of the loading. The resin was thoroughly washed sequentially with DMA and DCM and dried in high vacuum at 40° C., affording P1 (9.615 g, 5.0 mmol, 0.52 mmol/g).

In some cases, the commercially available H-Gly-2-Cl-Trt resin with loading 0.30-0.90 mmol/g was used.

### 7.1.2 Synthesis of H-Gly-Trt-Amide-MBHA Resin (P2)

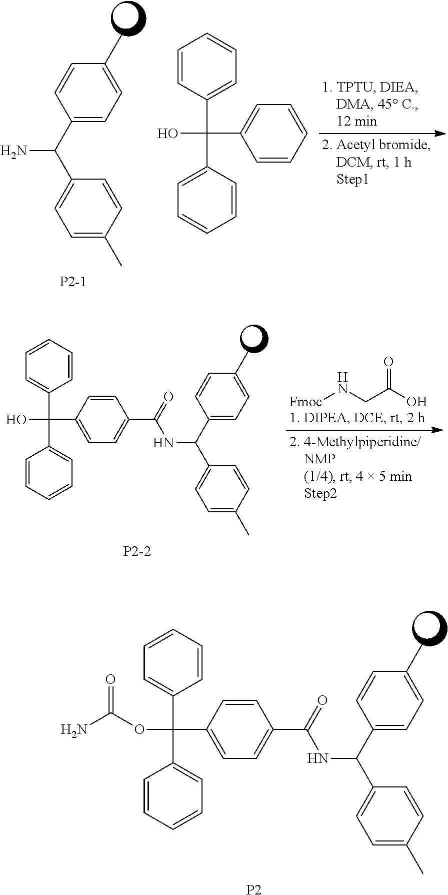

### Scheme 7.1.2. Synthesis of H-Gly-Trt-Amide-MBHA Resin P2

### Step 1. P2-2

Step 1-1: MBHA resin P2-1 (6.0 g, 8.46 mmol, loading 1.41 mmol/g, HCl salt) was carefully washed with DCM (+0.3% DIEA) and drained. A clear solution of 4-(hydroxydiphenylmethyl)benzoic acid (CAS 19672-49-2, 3.24 g, 1.26 eq., 10.6 mmol), TPTU (3.21 g, 1.24 eq., 10.5 mmol) and DIEA (4.37 g, 5.89 mL, 4 eq., 33.84 mmol) in DMA (16 mL) (pre-activated at 45° C. for 6 min) was added and the mixture was stirred at rt for 12 min, then drained and washed with DCM (+0.3% DIEA) (2×), PE (2×), DCM (2×), and PE (2×). The resin was dried in vacuo.

Step 1-2: A solution of acetyl bromide (10.4 g, 6.30 mL, 10.0 eq., 84.6 mmol) in DCM (37 mL) was added to the resin and the mixture was stirred at rt for 54 min. The resin was drained, washed with PE and dried in vacuo to afford P2-2.

### Step 2. P2

A clear solution of Fmoc-Gly-OH (1.847 g, 3 eq., 6.18 mmol) and DIEA (1.60 g, 2.16 mL, 6 eq., 12.36 mmol) in DCE (10 mL) was added to the resin P2-2 (2.0 g, 2.06 mmol) and the suspension was shaken for 2 h at rt. The resin was drained and was washed with DCM (3×). Capping was performed by shaking with DMA/Ac2O/pyridine (8/1/1) (5 mL) for 15 min at rt. The resin was drained and washed with DCM (2×), pentane (2×) and again DCM (2×). For Fmoc removal and determination of the loading the resin was shaken repeatedly with 4-methylpiperidine/NMP (1/4). The combined solutions were diluted with MeOH and used for the determination of the loading. The resin was thoroughly washed sequentially with DCM (2×), pentane (2×), DCM (2×) and pentane (3×) and dried in vacuum affording P2 (1.895 g, 0.921 mmol, 0.486 mmol/g).

### 7.1.3 Synthesis of H-Gly-HMPB-Bal-MBHA Resin (P3)

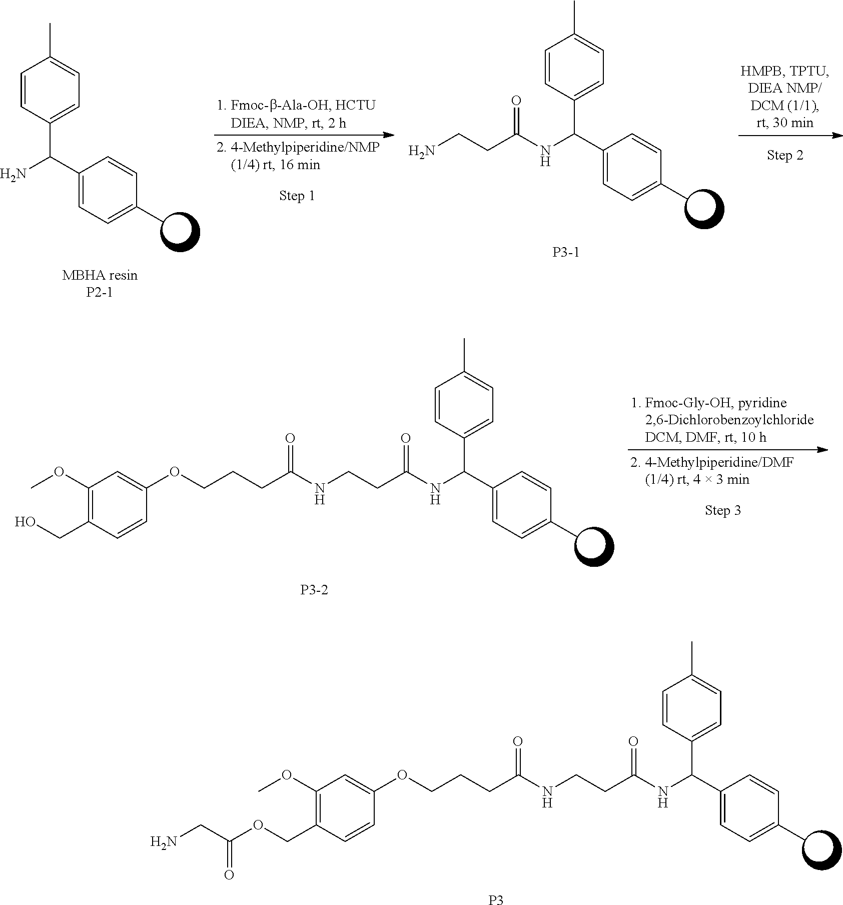

### Scheme 7.1.3. Synthesis of H-Gly-HMPB-Bal-MBHA Resin (P3)

### Step 1. P3-1

Step 1-1: MBHA resin P2-1 (20.2 g, 24.2 mmol, loading 1.2 mmol/g) was pre-swollen in DCM (+0.3% DIEA) (2×) and drained. A solution of Fmoc-p-Ala-OH (9.66 g, 1.28 eq., 31.0 mmol), HCTU (11.8 g, 1.18 eq., 28.6 mmol) and DIEA (18.9 g, 25.5 mL, 6 eq., 145 mmol) in NMP (75 mL) was added. The suspension was mixed at rt for 0.5 h and the resin was drained. A solution of Fmoc-3-Ala-OH (3.02 g, 0.4 eq., 9.7 mmol), HCTU (3.81 g, 0.38 eq., 9.21 mmol) and DIEA (6.27 g, 8.51 mL, 2 eq., 48.5 mmol) in NMP (25 mL) and NMP (25 mL) were added. The suspension was mixed at rt for 1.5 h. The resin was drained and washed with DCM (+0.3% DIEA). Capping was performed with DMA/Ac2O/pyridine (8/1/1) (60 mL) and DCM (+0.3% DIEA) (40 mL) at rt for 3 min. The resin was drained and washed with DCM (+0.3% DIEA) and PE. The resin was dried in vacuo for 16 h to afford Fmoc-β-Ala-MBHA resin.

Step 1-2. For Fmoc removal, the resin was treated with 4-methylpiperidine/NMP (1/4) (1×10 min, 1×6 min). The resin was washed with 2-propanol (+0.3% DIEA) (2×) and DCM (+0.3% DIEA) (4×) to yield P3-1. The combined deprotection and washing solutions were diluted with MeOH and used for the determination of the loading of P3-1 (25.3 mmol, 104% yield).

### Step 2. P3-2

To P3-1 (25.3 mmol) was added a solution of HMPB (CAS 136849-75-7, 6.98 g, 1.15 eq., 29.1 mmol), TPTU (8.25 g, 1.10 eq., 27.8 mmol) and DIEA (13.1 g, 17.7 mL, 4 eq., 101 mmol) in NMP/DCM (1/1) (70 mL) and the mixture was stirred for 12 min at rt. A second portion of DIEA (6.56 g, 8.84 mL, 2 eq., 50.5 mmol) was added to the mixture and the suspension was mixed at rt for 18 min. The resin was drained, washed with DCM (+0.3% DIEA) (1×), 2-propanol (+0.3% DIEA) (1×), DCM (+0.3% DIEA) (1×), PE, DCM (1×) and PE (3×). The resin was dried in high vacuum for 1 h to afford P3-2 (29.8 g, 25.3 mmol, loading 0.848 mmol/g).

### Step 3. P3

Step 3-1: A clear solution of Fmoc-Gly-OH (4.47 g, 2 eq., 15.0 mmol) and pyridine (2.50 g, 2.54 mL, 4.2 eq., 31.5 mmol) dissolved in DCM (19 mL) and DMF (19 mL) was added to P3-2 (8.86 g, 7.51 mmol, loading 0.848 mmol/g). To the mixed suspension at rt a solution of 2,6-dichlorobenzoylchloride (3.46 g, 2.37 mL, 2.2 eq., 16.5 mmol) in DMF (3.5 mL) was added continuously during 10 h using a syringe pump. The resin was drained, washed with DCM (0.3% DIEA) (2×), PE (2×), DCM (3×) and PE (3×), then dried in high vacuum (12.61 g, 7.51 mmol).

Step 3-2: The resin (12.13 g, 7.22 mmol) was treated with DCM, then capped with DMA/Ac20/Pyridine (8/1/1) (50 mL) at 45° C. for 8 min. The resin was drained and washed with DCM (0.3% DIEA) (2×), PE (2×) and DCM (0.3% DIEA) (3×). For Fmoc removal the resin was repeatedly treated at rt with 4-methylpiperidine/DMF (1/4) (4×3 min, each time 40 mL) and washed with DCM (0.3% DIEA) (2×), PE (2×), DCM (2×) and PE (2×). The resin was dried in high vacuum affording H-Gly-HMPB-Bal-MBHA resin P3 (9.31 g, 7.22 mmol, loading 0.776 mmol/g).

### 7.2 Fmoc-Amino Acid Building Blocks

TABLE 7.1
Examples of amino acid building blocks:

| | | | | Reference or |
|---|---|---|---|---|
| No | Abbreviation | Structure | Name | LC-MS |
| BB1 | Fmoc- NMePhe-OH | 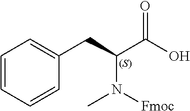 | N-(((9H-fluoren-9- yl)methoxy)carbon yl)-N-methyl-L- phenylalanine | Commercially available (CAS 77128- 73-5) |
| BB2 | Fmoc- NMecHexA- OH | 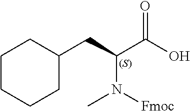 | (S)-2-((((9H- fluoren-9- yl)methoxy)carbonyl) (methyl)amino)- 3- cyclohexylpropanoic acid | Commercially available (CAS 148983-03-3) |
| BB3 | Fmoc- NMecPenA- OH | 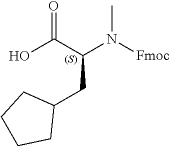 | (S)-2-((((9H- fluoren-9- yl)methoxy)carbonyl) (methyl)amino)- 3- cyclopentylpropanoic acid | JP6880352 |
| BB4 | Fmoc-cBuA- OH | 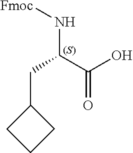 | (S)-2-((((9H- fluoren-9- yl)methoxy)carbonyl) amino)-3- cyclobutylpropanoic acid | Commercially available (CAS 478183-62-9) |
| BB5 | Fmoc- NMecBuA- OH | 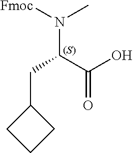 | (S)-2-((((9H- fluoren-9- yl)methoxy)carbonyl) (methyl)amino)- 3- cyclobutylpropanoic acid | Commercially available (CAS 2407819-94- 5) |
| BB6 | Fmoc- NMe3PyA- OH | 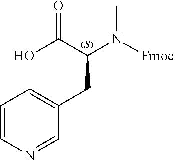 | (S)-2-((((9H- fluoren-9- yl)methoxy)carbonyl) (methyl)amino)- 3-(pyridin-3- yl)propanoic acid | Commercially available (CAS 1979173-93- 7) |
| BB7 | Fmoc-4PyA- OH | 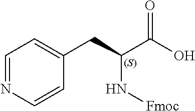 | (S)-2-((((9H- fluoren-9- yl)methoxy)carbonyl) amino)-3- (pyridin-4- yl)propanoic acid | Commercially available (CAS 169555-95-7) |
| BB8 | Fmoc- F(4OMe)-OH | 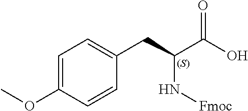 | (S)-2-((((9H- fluoren-9- yl)methoxy)carbonyl) amino)-3-(4- methoxyphenyl) propanoic acid | Commercially available (CAS 77128- 72-4) |
| BB9 | Fmoc- F(4OEt)-OH | 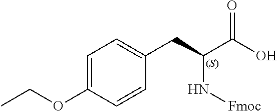 | (S)-2-((((9H- fluoren-9- yl)methoxy)carbonyl) amino)-3-(4- ethoxyphenyl) propanoic acid | Commercially available (CAS 119894-20-1) |
| BB10 | Fmoc- F(3OH)-OH | 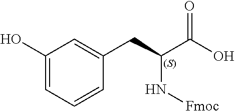 | (S)-2-((((9H- fluoren-9- yl)methoxy)carbonyl) amino)-3-(3- hydroxyphenyl) propanoic acid | Commercially available (CAS 178432-48-9) |
| BB11 | Fmoc-Sar-OH | 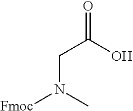 | N-(((9H-fluoren-9- yl)methoxy) carbonyl)-N- methylglycine | Commercially available (CAS 77128- 70-2) |
| BB12 | Fmoc- NMeY(tBu)- OH | 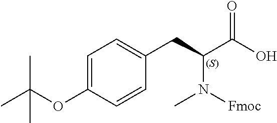 | (S)-2-((((9H- fluoren-9- yl)methoxy)carbonyl) (methyl)amino)- 3-(4-(tert- butoxy)phenyl) propanoic acid | Commercially available (CAS 133373-24-7) |
| BB13 | Fmoc-D- NMeA-OH | 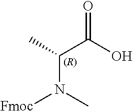 | N-(((9H-fluoren-9- yl)methoxy)carbonyl)- N-methyl-D- alanine | Commercially available (CAS 138774-92-2) |
| BB14 | Fmoc-D-Ala- OH | 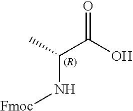 | (((9H-fluoren-9- yl)methoxy)carbonyl)- D-alanine | Commercially available (CAS 79990- 15-1) |
| BB15 | Fmoc-D-Nle- OH | 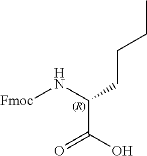 | (R)-2-((((9H- fluoren-9- yl)methoxy)carbonyl) amino)hexanoic acid | Commercially available (CAS 112883-41-7) |
| BB16 | Fmoc-NBnG- OH | 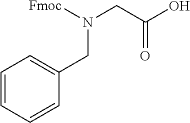 | N-(((9H-fluoren-9- yl)methoxy)carbonyl)- N- benzylglycine | Commercially available (CAS 141743-13-7) |
| BB17 | Fmoc- aMeD(OtBu)- OH | 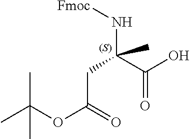 | (S)-2-((((9H- fluoren-9- yl)methoxy)carbonyl) amino)-4-(tert- butoxy)-2-methyl- 4-oxobutanoic acid | Commercially available (CAS 1072845-47- 6) |
| BB18 | Fmoc-Ahp- OH | 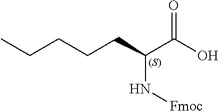 | (S)-2-((((9H- fluoren-9- yl)methoxy)carbonyl) amino)heptanoic acid | Commercially available (CAS 1197020-22- 6) |
| BB19 | Fmoc-Aoc- OH | 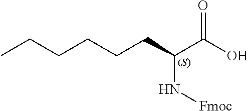 | (S)-2-((((9H- fluoren-9- yl)methoxy)carbonyl) amino)octanoic acid | Commercially available (CAS 888725-91-5) |
| BB20 | Fmoc- Aoc(3R-OH)- OH | 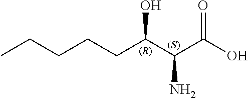 | (2S,3R)-2-amino-3- hydroxyoctanoic acid | Synthesized as described below, LCMS method BB-6 t_{R} = 1.30 min; [M + H]⁺ = 398.3 |
| BB21 | Fmoc-hS(Pr)- OH | 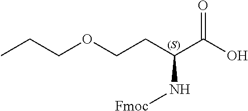 | N-(((9H-fluoren-9- yl)methoxy)carbonyl)- O-propyl-L- homoserine | Synthesized as described below, LC-MS method BB- 2, t_{R} = 0.608 min; [M + H]⁺ = 384.0 |
| BB22 | Fmoc-D-Pro- OH | 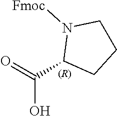 | (((9H-fluoren-9- yl)methoxy)carbon yl)-D-proline | Commercially available (CAS 101555-62-8) |
| BB23 | Fmoc-D- trans4Hyp(Ot Bu)-OH | 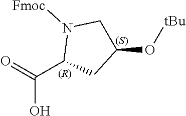 | (2R,4S)-1-(((9H- fluoren-9- yl)methoxy)carbonyl)- 4-(tert- butoxy)pyrrolidine- 2-carboxylic acid | Commercially available (CAS 268729-12-0) |
| BB24 | Fmoc-Pi(2R- COOH)-OH | 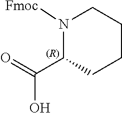 | (R)-1-(((9H- fluoren-9- yl)methoxy)carbonyl) piperidine-2- carboxylic acid | Commercially available (CAS 101555-63-9) |
| BB25 | Fmoc- Mor(3R- COOH)-OH | 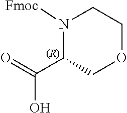 | (R)-4-(((9H- fluoren-9- yl)methoxy)carbon yl)morpholine-3- carboxylic acid | Commercially available (CAS 942153-03-9) |
| BB26 | Fmoc-D- aMeP-OH | 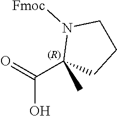 | (R)-1-(((9H- fluoren-9- yl)methoxy) carbonyl)-2- methylpyrrolidine- 2-carboxylic acid | Commercially available (CAS 1286768-33- 9) |
| BB27 | Fmoc- hSer(Trt)-OH | 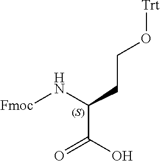 | N-(((9H-fluoren-9- yl)methoxy)carbonyl)- O-trityl-L- homoserine | Commercially available (CAS 111061-55-3) |
| BB28 | Fmoc-K(Ac)- OH | 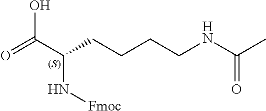 | N²-(((9H-fluoren-9- yl)methoxy)carbonyl)- N⁶-acetyl-L- lysine | Commercially available (CAS 159766-56-0) |
| BB29 | Fmoc-D- K(Ac)-OH | 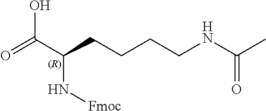 | N²-(((9H-fluoren-9- yl)methoxy)carbonyl)- N⁶-acetyl-D- lysine | Commercially available (CAS 320410-22-8) |
| BB30 | Fmoc-PEG10- OH | 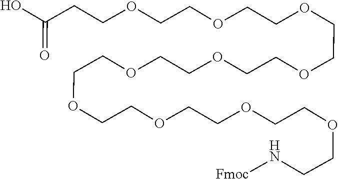 | 1-(9H-fluoren-9- yl)-3-oxo- 2,7,10,13,16,19,22, 25,28,31,34- undecaoxa-4- azaheptatriacontan- 37-oic acid | Commercially available (CAS 2101563-45- 3) |
| BB31 | Fmoc- W(60TBS)(N Boc)-OH | 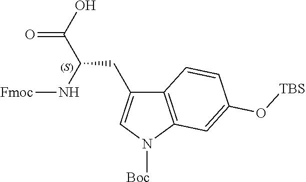 | (S)-2-((((9H- fluoren-9- yl)methoxy)carbonyl) amino)-3-(1- (tert- butoxycarbonyl)-6- ((tert- butyldimethylsilyl) oxy)-1H-indol-3- yl)propanoic acid | Synthesized as described below, LC-MS method BB- 4, t_{R} = 0.66 min; [M − Boc + H]⁺ = 557.4 |
| BB32 | Fmoc-W(7F)- OH | 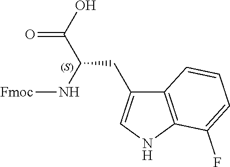 | (S)-2-((((9H- fluoren-9- yl)methoxy)carbonyl) amino)-3-(7- fluoro-1H-indol-3- yl)propanoic acid | Commercially available (CAS 1956434-65- 3) |
| BB33 | Fmoc-W(7N)- OH | 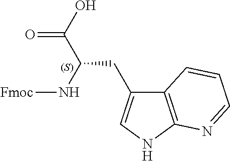 | (S)-2-((((9H- fluoren-9- yl)methoxy)carbonyl) amino)-3-(1H- pyrrolo[2,3- b]pyridin-3- yl)propanoic acid | Commercially available (CAS 737007-45-3) |
| BB34 | Fmoc- W(7N)(NBoc)- OH | 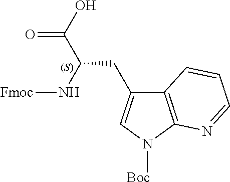 | (S)-2-((((9H- fluoren-9- yl)methoxy)carbonyl) amino)-3-(1- (tert- butoxycarbonyl)- 1H-pyrrolo[2,3- b]pyridin-3- yl)propanoic acid | Synthesized as described below, LC-MS method BB- 2, t_{R} = 0.44 min; [M + H]⁺ = 528.1 |
| BB35 | Fmoc- W(7Cl)-OH | 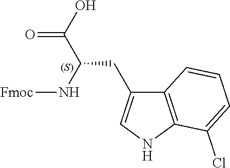 | (S)-2-((((9H- fluoren-9- yl)methoxy)carbonyl) amino)-3-(7- chloro-1H-indol-3- yl)propanoic acid | Commercially available (CAS 2382374-53- 8) |
| BB36 | Fmoc- W(7Cl)(NBoc)- OH | 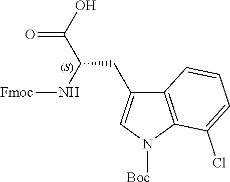 | (S)-2-((((9H- fluoren-9- yl)methoxy)carbonyl) amino)-3-(1- (tert- butoxycarbonyl)-7- chloro-1H-indol-3- yl)propanoic acid | Commercially available (CAS 1956436-89- 7) |
| BB37 | Fmoc- W(7OMe)-OH | 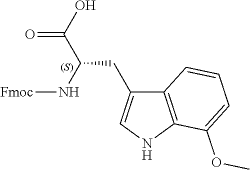 | (S)-2-((((9H- fluoren-9- yl)methoxy)carbonyl) amino)-3-(7- methoxy-1H-indol- 3-yl)propanoic acid | Commercially available (CAS 2416720-26- 6) |
| BB38 | Fmoc- F(4OEtNHBoc)- OH | 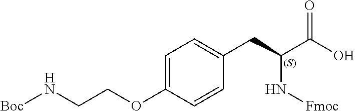 | (S)-2-((((9H- fluoren-9- yl)methoxy)carbonyl) amino)-3-(4-(2- ((tert- butoxycarbonyl) amino)ethoxy)phenyl) propanoic acid | Commercially available (CAS 1013883-02- 7) |
| BB39 | Fmoc- F(4amNHBoc)- OH | 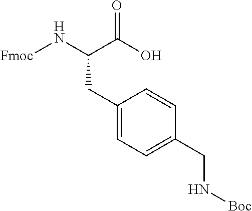 | (S)-2-((((9H- fluoren-9- yl)methoxy)carbonyl) amino)-3-(4- (((tert- butoxycarbonyl) amino)methyl)phenyl) propanoic acid | Commercially available (CAS 204715-91-3) |
| BB40 | Fmoc- F(4amPEG2N HBoc)-OH | 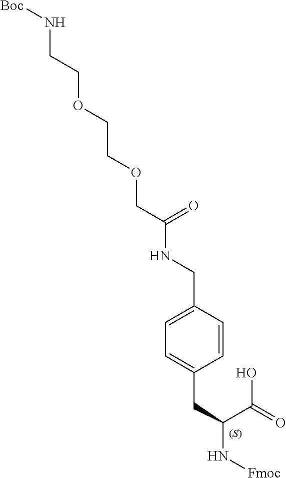 | (S)-2-((((9H- fluoren-9- yl)methoxy)carbonyl) amino)-3-(4- (14,14-dimethyl- 3,12-dioxo-5,8,13- trioxa-2,11- diazapentadecyl) phenyl)propanoic acid | Synthesized as described below, LC-MS method BB- 1, t_{R} = 5.63 min; [M + H]⁺ = 662.3 |
| BB41 | Fmoc- K(COpipzaa) (OtBu)-OH | 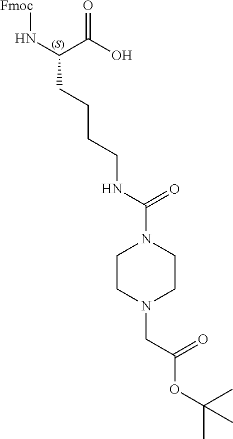 | N²-(((9H-fluoren-9- yl)methoxy)carbonyl)- N⁶-(4-(2-(tert- butoxy)-2- oxoethyl)piperazine- 1-carbonyl)-L- lysine | Synthesized as described below, LCMS method BB- 5, t_{R} = 1.44 min; [M + H]⁺ = 595.5 |
| BB42 | Fmoc- MeK(COpipzaa) (OtBu)-OH | 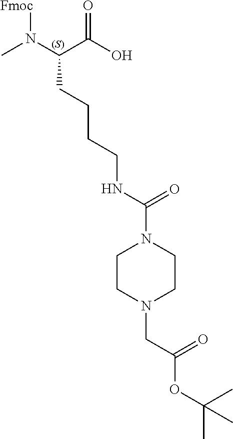 | N²-(((9H-fluoren-9- yl)methoxy)carbonyl)- N⁶-(4-(2-(tert- butoxy)-2- oxoethyl)piperazine- 1-carbonyl)-N²- methyl-L-lysine | Synthesized as described below, LCMS method BB- 5, t_{R} = 1.38 min; [M + H]⁺ = 609.4 |
| BB43 | Fmoc- 3PyA(6NHBoc)- OH | 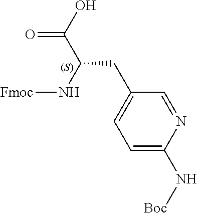 | (S)-2-((((9H- fluoren-9- yl)methoxy)carbonyl) amino)-3-(6- ((tert- butoxycarbonyl) amino)pyridin-3- yl)propanoic acid | WO2021167 107 |
| BB44 | Fmoc- Q(Pyrro(2CN 4F2))-Oh | 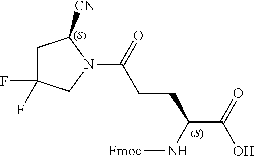 | (S)-2-((((9H- fluoren-9- yl)methoxy)carbonyl) amino)-5-((S)-2- cyano-4,4- difluoropyrrolidin- 1-yl)-5- oxopentanoic acid | Synthesized as described below, LC-MS method BB- 1, t_{R} = 4.79 min; [M + NH₄]⁺ = 501.1 |
| BB45 | Fmoc- Asp(OMpe)- OH | 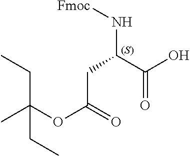 | (S)-2-((((9H- fluoren-9- yl)methoxy)carbonyl) amino)-4-((3- methylpentan-3- yl)oxy)-4- oxobutanoic acid | Commercially available (CAS 180675-08-5) |
| BB46 | Fmoc- Lys(Alloc)- OH | 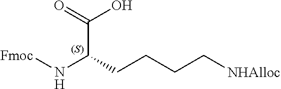 | N²-(((9H-fluoren-9- yl)methoxy)carbonyl)- N⁶- ((allyloxy)carbonyl)- L-lysine | Commercially available (CAS 146982-27-6) |
| BB47 | Fmoc- Glu(OAll)-OH | 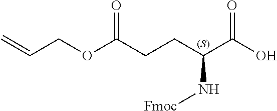 | (S)-2-((((9H- fluoren-9- yl)methoxy)carbonyl) amino)-5- (allyloxy)-5- oxopentanoic acid | Commercially available (CAS 133464-46-7) |
| BB51 | Fmoc- F(4CH₂N₃)- OH | 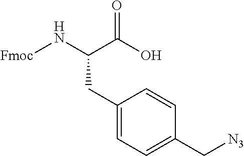 | (S)-2-((((9H- fluoren-9- yl)methoxy)carbonyl) amino)-3-(4- (azidomethyl)phen yl)propanoic acid | Commercially available (CAS 2375587-79- 2) |
| BB52 | Fmoc- F4(MeTriazol ylMeNHBoc)- OH | 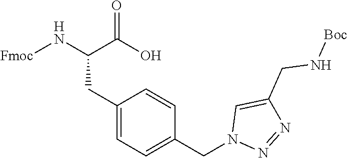 | (S)-2-((((9H- fluoren-9- yl)methoxy)carbonyl) amino)-3-(4-((4- (((tert- butoxycarbonyl) amino)methyl)-1H- 1,2,3-triazol-1- yl)methyl)phenyl) propanoic acid | Synthesized as described below, LC-MS method BB- 1, t_{R} = 5.55 min; [M + H]⁺ = 598.2 |
| BB59 | Fmoc- F(4OEtNHAlloc)- OH | 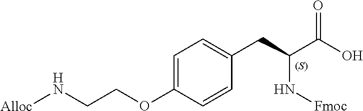 | (S)-2-((((9H- fluoren-9- yl)methoxy)carbonyl) amino)-3-(4-(2- (((allyloxy)carbonyl) amino)ethoxy) phenyl)propanoic acid | Synthesized as described below, LC-MS method BB- 6, t_{R} = 1.18 min; [M + H]⁺ = 531.2 |

Certain amino acids were synthesized as described below.

### 7.2.1 Synthesis of (2S,3R)-2-((((9H-fluoren-9-yl)methoxy)carbonyl)amino)-3-hydroxyoctanoic acid (BB20)

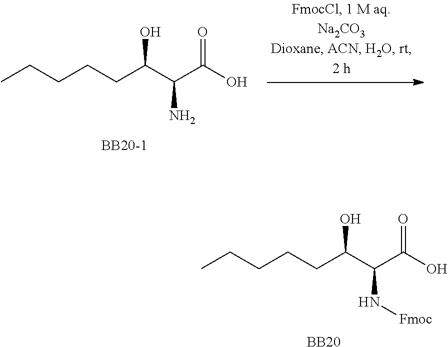

### Scheme 7.2.1. Synthesis of BB20

To a slurry of BB20-1 (165.0 mg, 1 eq., 941.6 μmol) in dioxane (3 mL) were added 1 M aq. Na₂CO₃ (299.4 mg, 2.825 mL, 1.00 molar, 3.0 Eq, 2.825 mmol), a solution of Fmoc-C₁ (243.6 mg, 1 eq., 941.6 μmol) in dioxane (3 mL), ACN (2 mL) and H₂O (2 mL). The reaction mixture was stirred for 75 min at rt, then H₂O (5 mL) was added and stirring at rt was continued for 45 min. The mixture was partitioned between EtOAc (50 mL) and 1 M aq. HCl (25 mL). The organic layer was washed with 500 aq. KHSO₄ (2×10 mL) and brine (10 mL), dried over Na₂SO₄, filtered and concentrated to dryness in vacuo. The crude product was purified by reversed-phase flash chromatography (Column: RediSep Gold C18Aq 100 g; Eluent A: H₂O+0.100 TFA and eluent B: ACN). Pure fractions were combined and after partial removal of ACN lyophilized to afford BB-20 (278 mg, 699 μmol, 74% yield) as a light beige solid. LCMS method BB-6, t_{R}=1.30 min; [M+H]⁺=398.3.

### 7.2.2 Synthesis of N-(((9H-fluoren-9-yl)methoxy)carbonyl)-O-propyl-L-homoserine (BB21)

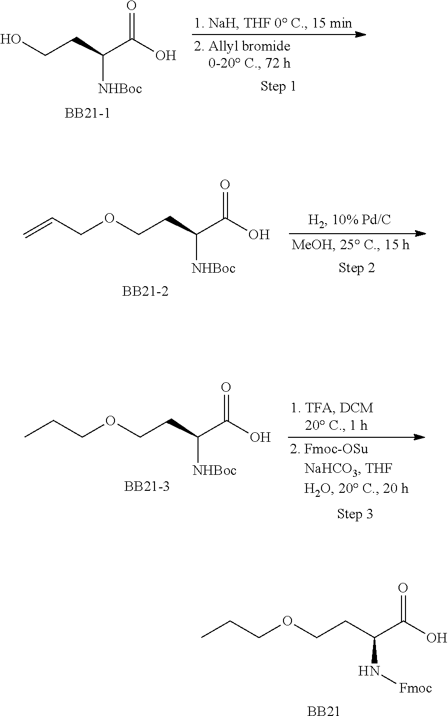

### Scheme 7.2.2. Synthesis of BB21

### Step 1. O-allyl-N-(tert-butoxycarbonyl)-L-homoserine (BB21-2)

To a solution of NaH (22.8 g, 570 mmol, 60%, 2.50 eq.) in THF (1000 mL) was added compound BB21-1 (50.0 g, 228 mmol, 1.00 eq.) at 0° C. under N₂ flow. The reaction mixture was stirred at 0° C. for 15 min and allyl bromide (55.1 g, 456 mmol, 2.00 eq.) was added at 0° C. Then the reaction mixture was stirred at 20° C. for 72 h under N₂. The reaction mixture was quenched by addition of H₂O (300 mL) at 0° C. under N₂ flow and concentrated under reduced pressure to remove THF. Then aqueous layer was washed with hexane (200 mL) and pH adjusted to 3 with 1M aq. HCl, and the product was extracted with EtOAc (200 mL×3). The combined organic layers were washed with brine (200 mL), dried over Na₂SO₄, filtered, and concentrated to dryness in vacuo. The residue was purified by column chromatography over silica gel (PE:EtOAc=100:1 to 1:1) to afford compound BB21-2 (33.1 g, 127 mmol, 55.9% yield) as a light-yellow oil. 1H NMR (400 MHz, CDCl₃) δ ppm 5.92-5.85 (m, 1H), 5.62 (d, 1H), 5.30-5.18 (m, 2H), 4.39 (d, 1H), 4.00-3.98 (m, 2H), 3.62-3.60 (m, 2H), 2.17-2.08 (m, 2H), 1.45 (s, 9H). MS (ESI−): m/z [M−H]⁻ 257.8.

### Step 2. N-(tert-butoxycarbonyl)-O-propyl-L-homoserine (BB21-3)

To a solution of compound BB21-2 (33.1 g, 127 mmol, 1.00 eq.) in MeOH (300 mL) was added 10% Pd/C (3.50 g) under N₂. The suspension was degassed and purged with H₂ for 3 times. The mixture was stirred under H₂ (50 Psi) at 25° C. for 15 h. The reaction mixture was filtered, and the filtrate was concentrated to dryness in vacuo to afford compound BB21-3 (32.7 g, 125 mmol, 98% yield) as a light-yellow oil. 1H NMR (400 MHz, CDCl₃) δ ppm 9.10 (s, 1H), 5.75 (d, 1H), 4.35 (d, 1H), 3.60 (s, 2H), 3.43-3.39 (m, 2H), 2.17-2.07 (m, 2H), 1.62-1.57 (m, 2H), 1.45 (s, 9H), 0.94-0.91 (m, 3H). MS (ESI−): m/z [M−H]⁻ 259.9.

### Step 3. N-(((9H-fluoren-9-yl)methoxy)carbonyl)-O-propyl-L-homoserine (BB21)

Step 3-1: To a solution of compound BB21-3 (32.7 g, 125 mmol, 1.00 eq.) in DCM (100 mL) was added TFA (154 g, 1.30 μmol, 100 mL, 96%, 10.3 eq.). The reaction mixture was stirred at 20° C. for 1 h. The reaction mixture was concentrated to dryness in vacuo.

Step 3-2: To the residue dissolved in H₂O (200 mL) were added NaHCO₃ (84.1 g, 1.00 mol, 8.00 eq.) and Fmoc-OSu (40.1 g, 118 mmol, 0.95 eq.) in THF (200 mL). The reaction mixture was stirred at 20° C. for 20 h. pH was adjusted to 2 by 1M aq. HCl and the product was extracted with EtOAc (150 mL×3). The combined organic layers were washed with H₂O (100 mL) and brine (100 mL), dried over Na₂SO₄, filtered, and concentrated to dryness in vacuo. The residue was purified by column chromatography over silica gel (PE:EtOAc=100:1 to 1:1) to afford BB21 (41.1 g, 102 mmol, 81.8% yield) as a white solid. 1H NMR (400 MHz, CDCl₃) δ ppm 7.77 (d, 2H), 7.62-7.59 (m, 2H), 7.42-7.39 (m, 2H), 7.33-7.30 (m, 2H), 6.12 (d, 1H), 4.45-4.35 (m, 3H), 4.25-4.22 (m, 1H), 3.64 (d, 2H), 3.47-3.43 (m, 2H), 2.21-2.05 (m, 2H), 1.65-1.60 (m, 2H), 0.97-0.93 (m, 3H). LC-MS method BB-2, t_{R}=0.608 min; [M+H]⁺=384.0.

### 7.2.3 Synthesis of (S)-2-((((9H-fluoren-9-yl)methoxy)carbonyl)amino)-3-(1-(tert-butoxycarbonyl)-7-((tert-butyldimethylsilyl)oxy)-1H-indol-3-yl)propanoic acid (BB31)

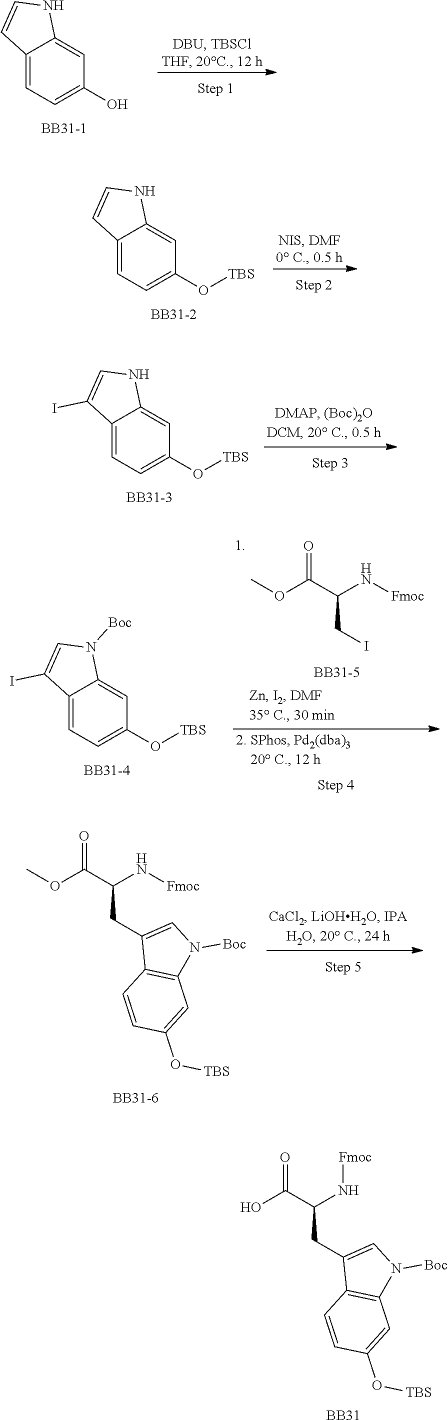

### Scheme 7.2.3. Synthesis of BB31

### Step 1. 6-((tert-butyldimethylsilyl)oxy)-1H-indole (BB31-2)

To a solution of compound BB31-1 (10.0 g, 75.1 mmol, 1 eq.) in THF (70 mL) was added DBU (13.7 g, 90.1 mmol, 13.58 mL, 1.2 eq.) and TBSCl (16.9 g, 112.66 mmol, 13.86 mL, 1.5 eq.). The mixture was stirred at 20° C. for 12 h. The reaction mixture was diluted with H₂O (200 mL) and the product was extracted with EtOAc (100 mL×3). The combined organic layers were washed with brine (50 mL×2), dried over Na₂SO₄, filtered, and concentrated to dryness in vacuo. The residue was purified by column chromatography over silica gel (PE:EtOAc=I/O to 0/1). Compound BB31-2 (18 g, 72.7 mmol) was obtained as a pink solid.

### Step 2. 6-((tert-butyldimethylsilyl)oxy)-3-iodo-1H-indole (BB31-3)

To a solution of compound BB31-2 (18.0 g, 72.7 mmol, 1 eq.) in DMF (126 mL) was added NIS (16.5 g, 73.4 mmol, 1.01 eq.) at 0° C. The mixture was stirred at 0° C. for 0.5 h. The reaction mixture was quenched by addition of sat. NaHCO₃ (200 mL), and then extracted with MTBE (150 mL×4). The combined organic layers were washed with Na₂S₂O₃ (100 mL×2), dried over Na₂SO₄, filtered, and concentrated to dryness in vacuo. The residue was purified by column chromatography over silica gel (PE:EtOAc=I/O to 0/1). Compound BB31-3 (27.0 g, 72.3 mmol) was obtained as a pink solid.

### Step 3. tert-butyl 6-((tert-butyldimethylsilyl)oxy)-3-iodo-1H-indole-1-carboxylate (BB31-4)

To a solution of compound BB31-3 (27.0 g, 72.3 mmol, 1 eq.) in DCM (150 mL) was added (Boc)₂O (25.2 g, 115 mmol, 26.5 mL, 1.6 eq.) and DMAP (441 mg, 3.62 mmol, 0.05 eq.). The mixture was stirred at 20° C. for 0.5 h. The reaction mixture was diluted with H₂O (300 mL) and the product was extracted with DCM (150 mL×3). The combined organic layers were dried over Na₂SO₄, filtered, and concentrated to dryness in vacuo. The residue was purified by column chromatography over silica gel (PE:EtOAc=1/0 to 0/1). Compound BB31-4 (31.0 g, 65.4 mmol) was obtained as a pink solid.

### Step 4. tert-butyl (S)-3-(2-((((9H-fluoren-9-yl)methoxy)carbonyl)amino)-3-methoxy-3-oxopropyl)-6-((tert-butyldimethylsilyl)oxy)-1H-indole-1-carboxylate (BB31-6)

All the operations were performed at a pre-dried flask under N₂.

Step 4-1: To a solution of Zn (10.7 g, 164 mmol, 3 eq.) in DMF (110 mL) was added I₂ (2.09 g, 8.24 mmol, 0.15 eq.) with stirring at 20° C. until the color disappeared. Then compound BB31-5 (27.2 g, 60.4 mmol, 1.1 eq.) and I₂ (2.09 g, 8.24 mmol, 0.15 eq.) were added and the mixture was stirred at 35° C. for 30 min.

Step 4-2: After that, a solution of compound BB31-4 (26.0 g, 54.9 mmol, 1 eq.) in DMF (110 mL), Pd₂(dba)₃ (1.26 g, 1.37 mmol, 0.025 eq.) and SPhos (1.13 g, 2.75 mmol, 0.05 eq.) were added and the mixture was stirred at 20° C. for 12 h. After completion, the reaction mixture was quenched by addition 0.1 M aq. HCl (500 mL) at 20° C. under N₂, and extracted with EtOAc (300 mL×3). The combined organic layers were washed with brine (300 mL×2), dried over Na₂SO₄, filtered, and concentrated to dryness in vacuo. The crude product was purified by reversed-phase chromatography (0.1% TFA) and then further purified by column chromatography over silica gel (PE:EtOAc=I/O to 0/1). Compound BB31-6 (18.0 g, 26.83 mmol) was obtained as yellow oil. LC-MS method BB-2, t_{R}=0.85 min; [M-Boc+H]⁺=571.4.

### Step 5. (S)-2-((((9H-fluoren-9-yl)methoxy)carbonyl)amino)-3-(1-(tert-butoxycarbonyl)-6-((tert-butyldimethylsilyl)oxy)-1H-indol-3-yl)propanoic acid (BB31)

To a solution of compound BB31-6 (18.0 g, 26.83 mmol, 1 eq.) in IPA (80 mL) and H₂O (40 mL) was added CaCl₂ (47.6 g, 429 mmol, 16 eq.) and LiOH·H₂O (4.50 g, 107.32 mmol, 4 eq.). The mixture was stirred at 20° C. for 24 h. After completion, the reaction mixture pH was adjusted to 5-6 by addition of citric acid at 20° C., and then the reaction mixture was diluted with H₂O (500 mL). The product was extracted with EtOAc (200 mL×3). The combined organic layers were washed with brine (300 mL×2), dried over Na₂SO₄, filtered, and concentrated to dryness in vacuo. The residue was purified by reversed-phase chromatography (Column: Luna C18, 100 Å, 10 μm, 70 mm×250 mm; Eluent A: H₂O+0.1% TFA and eluent B: ACN) to afford compound BB31 (14.1 g, 21.2 mmol, 79.2% yield) as a yellow solid. 1H NMR (400 MHz, DMSO-d₆) δ ppm 12.70-12.95 (m, 1H) 7.33-7.89 (m, 10H) 7.18-7.29 (m, 2H)6.74-6.83 (m, 1H)4.23-4.33 (m, 1H)4.09 (brs, 3H)3.08-3.18 (m, 1H) 2.93-3.03 (m, 1H) 1.49-1.62 (m, 9H) 0.89-0.98 (m, 9H) 0.17 (s, 6H). LC-MS method BB-4, t_{R}=0.66 min; [M−Boc+H]=⁺557.4.

### 7.2.4 Synthesis of (S)-2-((((9H-fluoren-9-yl)methoxy)carbonyl)amino)-3-(1-(tert-butoxycarbonyl)-1H-pyrrolo[2,3-b]pyridin-3-yl)propanoic acid (BB34)

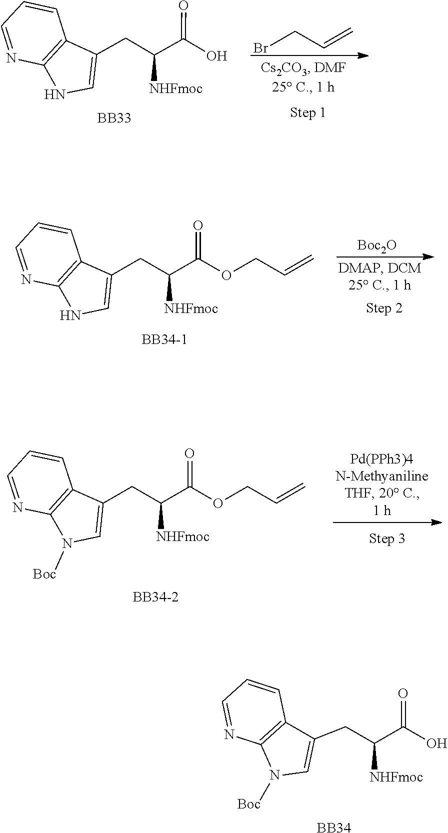

### Scheme 7.2.4. Synthesis of BB34

### Step 1. Allyl (S)-2-((((9H-fluoren-9-yl)methoxy)carbonyl)amino)-3-(1H-pyrrolo[2,3-b]pyridin-3-yl)propanoate (BB34-1)

To a solution of compound BB33 (180.0 g, 420 mmol, 1.00 eq.) in DMF (1800 mL) were added Cs₂CO₃ (68.6 g, 210 mmol, 0.50 eq.) and allyl bromide (56.0 g, 464 mmol, 1.10 eq.), the reaction mixture was stirred at 25° C. for 1 h. Then the product was extracted with EtOAc (1000 mL×3). The combined organic layers were washed with water (2000 mL×3) and brine (500 mL×3), dried over Na₂SO₄, filtered, and concentrated to dryness in vacuo (200 g). The crude product was triturated with MTBE (1500 mL) at 25° C. for 16 h. Compound BB34-1 (140 g, 289 mmol, 68.7% yield) was obtained as yellow solid. 1H NMR (400 MHz, DMSO-d₆) δ ppm 11.58-11.29 (m, 1H), 8.22-8.16 (m, 1H), 7.97 (t, 2H), 7.88 (d, 2H), 7.64 (t, 2H), 7.41 (d, 2H), 7.35-7.24 (m, 2H), 7.03 (dd, 1H), 5.90-5.74 (m, 1H), 5.31-5.11 (m, 2H), 4.63-4.46 (m, 2H), 4.35 (td, 1H), 4.29-4.14 (m, 3H), 3.25-3.16 (m, 1H), 3.14-3.04 (m, 1H). LC-MS method BB-2, t_{R}=0.49 min; [M+H]⁺=468.1.

### Step 2. tert-Butyl (S)-3-(2-((((9H-fluoren-9-yl)methoxy)carbonyl)amino)-3-(allyloxy)-3-oxopropyl)-1H-pyrrolo[2,3-b]pyridine-1-carboxylate (BB34-2)

To a solution of compound BB34-1 (140 g, 289 mmol, 1.00 eq.) in DCM (1.40 L) was added DMAP (3.54 g, 28.9 mmol, 0.10 eq.) and Boc₂O (72.7 g, 334 mmol, 76.5 mL, 1.15 eq.). The reaction mixture was stirred at 25° C. for 1 h. The reaction mixture was diluted with H₂O (1.40 L) and the product was extracted with DCM (500 mL×3). The combined organic layers were washed with brine (500 mL×2), dried over Na₂SO₄, filtered, and concentrated to dryness in vacuo. The residue was purified by column chromatography over silica gel (PE:EtOAc=100/1 to 0/1) to afford compound BB34-2 (81.0 g, 138 mmol, 47.4% yield) as a white solid. 1H NMR (400 MHz, DMSO-d₆) δ ppm 8.38 (d, 1H), 8.11-7.96 (m, 2H), 7.87 (d, 2H), 7.68 (s, 1H), 7.60 (t, 2H), 7.39 (t, 2H), 7.31-7.21 (m, 3H), 5.93-5.76 (m, 1H), 5.35-5.12 (m, 2H), 4.57 (d, 2H), 4.48-4.37 (m, 1H), 4.29-4.10 (m, 3H), 3.23-3.15 (m, 1H), 3.15-3.02 (m, 1H), 1.56 (s, 9H). LC-MS method BB-2, t_{R}=0.54 min; [M+H]⁺=568.1.

### Step 3. (S)-2-((((9H-fluoren-9-yl)methoxy)carbonyl)amino)-3-(1-(tert-butoxycarbonyl)-1H-pyrrolo[2,3-b]pyridin-3-yl)propanoic acid (BB34)

To a solution of compound BB34-2 (81.0 g, 138 mmol, 1.00 eq.) in THF (800 mL) was added N-methylaniline (29.6 g, 276 mmol, 29.9 mL, 2.00 eq.) and Pd(PPh₃)₄ (7.97 g, 6.90 mmol, 0.05 eq.). The reaction mixture was stirred at 20° C. for 1 h. The reaction mixture was diluted with H₂O (800 mL) and the product was extracted with ethyl acetate (400 mL×3). The combined organic layers were washed with brine (300 mL×3), dried over Na₂SO₄, filtered, and concentrated to dryness in vacuo. The residue was purified by reversed-phase chromatography (neutral conditions) to afford compound BB34 (76.0 g, 123 mmol, 88.8% yield) as a white solid. 1H NMR (400 MHz, DMSO-d₆) δ ppm 13.51-12.18 (m, 1H), 8.43-8.32 (m, 1H), 8.09 (d, 1H), 7.91-7.78 (m, 3H), 7.67 (s, 1H), 7.60 (dd, 2H), 7.41-7.37 (m, 2H), 7.31-7.19 (m, 3H), 4.35-4.26 (m, 1H), 4.22-4.12 (m, 3H), 3.20 (dd, 1H), 3.09-2.98 (m, 1H), 1.55 (s, 9H). LC-MS method BB-2, t_{R}=0.44 min; [M+H]⁺=528.1.

### 7.2.5 Synthesis of (S)-2-((((9H-fluoren-9-yl)methoxy)carbonyl)amino)-3-(4-(14,14-dimethyl-3,12-dioxo-5,8,13-trioxa-2,11-diazapentadecyl)phenyl)propanoic acid (BB40)

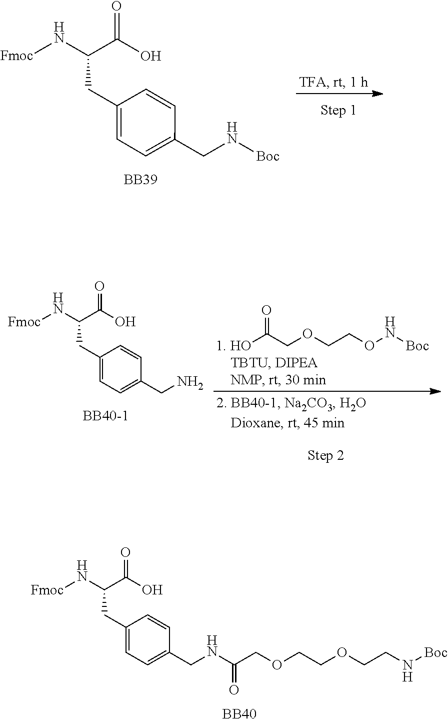

### Scheme 7.2.5. Synthesis of BB40

### Step 1. (S)-2-((((9H-fluoren-9-yl)methoxy)carbonyl)amino)-3-(4-(aminomethyl)phenyl)propanoic acid (BB40-1)

BB39 (5.166 g, 10.0 mmol, 1 eq.) was dissolved in 95% aq. TFA (50 mL). The reaction was stirred at rt for 1 h, then concentrated to dryness in vacuo. The residue was suspended in toluene/DCM and the mixture was concentrated to dryness in vacuo. This treatment was repeated once and BB40-1 (6.73 g, 10 mmol, crude, TFA salt) was obtained as a yellow foam. The product was used for the next step directly without further purification. LC-MS method BB-1, t_{R}=3.60 min; [M+H]⁺=417.2.

### Step 2. (S)-2-((((9H-fluoren-9-yl)methoxy)carbonyl)amino)-3-(4-(14,14-dimethyl-3,12-dioxo-5,8,13-trioxa-2,11-diazapentadecyl)phenyl)propanoic acid (BB40)

To compound BB40-1 (6.73 g, 10 mmol, crude, TFA salt) were added dioxane (60 mL) and aq. Na₂CO₃ (0.5 M, 60 mL, 30 mmol, 3 eq.). Then a solution of Boc-8-amino-3,6-dioxaoctanoic acid (3.69 g, 14.0 mmol, 1.4 eq., CAS 108466-89-3), TBTU (4.50 g, 14.0 mmol, 1.4 eq.) and DIPEA (2.07 g, 16 mmol, 2.77 mL, 1.6 eq.) in NMP (45 mL) (preactivation time 30 min) was added. The mixture was stirred at rt for 45 min, then dioxane was removed in vacuo. The residue was partitioned between EtOAc (250 mL) and 0.5 M aq. HCl (125 mL). The organic layer was washed with 5% aq. KHSO₄ (3×50 mL) and brine (50 mL), dried over Na₂SO₄, filtered, and concentrated to dryness in vacuo. The residue was purified by column chromatography over silica gel (Eluent A: heptanes +1% AcOH, eluent B: EtOAc+1% AcOH). Pure fractions were combined and concentrated to dryness in vacuo. The residue was dissolved in EtOAc (300 mL). The organic layer was washed with 5% aq. Na₂CO₃ (4×15 mL), 5% aq. KHSO₄ (50 mL)/brine (15 mL) and brine (15 mL), dried over Na₂SO₄, filtered, and concentrated to dryness in vacuo and dried at 40° C. in high vacuum to give compound BB40 (5.9 g, 8.7 mmol, 87% yield) as a white foam. 1H NMR (600 MHz, DMSO-d6) δ 12.77 (br s, 1H), 8.21 (br t, 1H), 7.88 (d, 2H), 7.72 (br d, 1H), 7.67-7.64 (dd, 2H), 7.41 (m, 2H), 7.30 (m, 2H), 7.21-7.16 (m, 4H), 6.79 (brt, 1H), 4.27-4.26 (brd, 2H), 4.23-4.16 (m, 3H), 4.14-4.01 (m, 1H), 3.92 (s, 2H), 3.58-3.56 (m, 2H), 3.53-3.51 (m, 2H), 3.32-3.29 (m, 1H), 3.06-3.01 (m, 3H), 2.84 (br dd, 1H), 1.35 (s, 9H). LC-MS method BB-1, t_{R}=5.63 min; [M+H]⁺=662.3.

### 7.2.6 Synthesis of N²-(((9H-fluoren-9-yl)methoxy)carbonyl)-N⁶-(4-(2-(tert-butoxy)-2-oxoethyl)piperazine-1-carbonyl)-L-lysine (BB41)

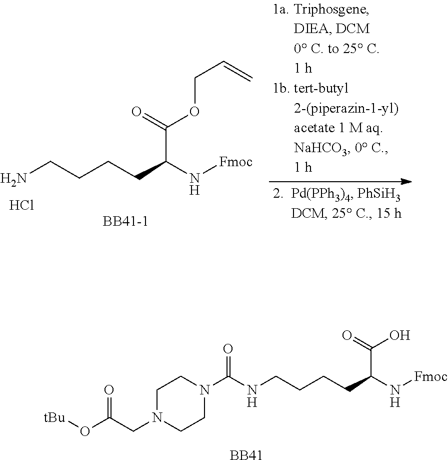

### Scheme 7.2.6. Synthesis of BB41

Step 1: To a solution of BB41-1 (CAS: 815619-80-8, 3.34 g, 7.5 mmol, HCl salt) and DIEA (4.18 mL, 24.00 mmol) in DCM (30 mL) at 0° C. was added triphosgene (1.00 g, 3.38 mmol). The mixture was stirred at 0° C. for 0.25 h, and then 25° C. for 0.75 h. To the mixture at 0° C. were added tert-butyl 2-(piperazin-1-yl)acetate (CAS: 112257-22-4, 1.65 g, 8.25 mmol) and 1 M aq. NaHCO₃ (50 mL). The mixture was then stirred at 0° C. for 1 h, and then the product was extracted with EtOAc. The organic layers were washed successively with 5% aq. AcOH, H₂O, and sat. aq. NaHCO₃, dried over Na₂SO₄, filtered and concentrated. The resulting residue was purified by silica gel flash column chromatography (gradient, hexane/EtOAc=100/0 to 0/100). Appropriate fractions were concentrated.

Step 2: To a solution of the resulting residue (2.13 g, 3.36 mmol) in DCM (30 mL) at 0° C. were added phenylsilane (0.48 mL, 3.90 mmol) and Pd(PPh₃)₄ (0.04 g, 0.04 mmol). The mixture was then stirred at 25° C. for 15 h, and then concentrated. The resulting residue was purified by silica gel flash column chromatography (gradient, hexane/EtOAc=50/50 to 0/100, then DCM/MeOH=100/0 to 85/15) to afford the title compound (1.51 g, 2.54 mmol). ¹H NMR (500 MHz, DMSO-d₆) δ 12.52 (br s, 1H), 7.90 (d, 2H), 7.73 (d, 2H), 7.68-7.55 (m, 1H), 7.42 (dd, 2H), 7.33 (dd, 2H), 6.45 (br 1H), 4.32-4.19 (m, 3H), 3.93-3.85 (m, 1H), 3.30-3.19 (m, 4H), 3.09 (s, 2H), 3.03-2.95 (m, 2H), 2.46-2.32 (m, 4H), 1.72-1.55 (m, 2H), 1.39 (s, 9H), 1.38-1.23 (m, 4H). LCMS method BB-5, t_{R}=1.44 min; [M+H]⁺=595.5.

### 7.2.7 Synthesis of N²-(((9H-fluoren-9-yl)methoxy)carbonyl)-N⁶-(4-(2-(tert-butoxy)-2-oxoethyl)piperazine-1-carbonyl)-N²-methyl-L-lysine (BB42)

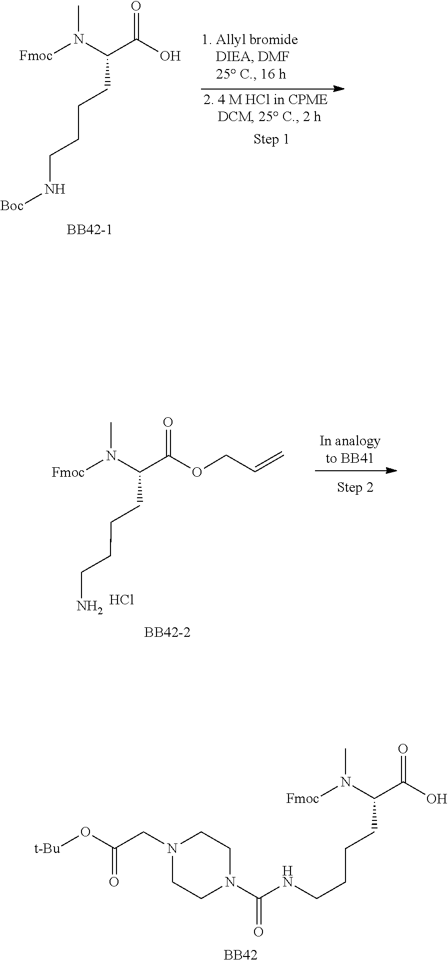

### Scheme 7.2.7. Synthesis of BB42

### Step 1. Prop-2-en-1-yl (2S)-6-amino-2-({[(9H-fluoren-9-yl)methoxy]carbonyl}(methyl)amino)-hexanoate hydrochloride (BB42-2)

Step 1-1: To a solution of BB42-1 (CAS: 197632-76-1, 12 g, 24.9 mmol) in DMF (99 mL) at 25° C. were added DIEA (6.51 mL, 37.3 mmol) and allyl bromide (2.37 mL, 27.4 mmol). The mixture was stirred at 25° C. for ca. 16 h. The reaction was quenched with H₂O. The product was extracted with EtOAc. The organic layer was dried over Na₂SO₄, filtered and concentrated. The resulting residue was purified by silica gel flash column chromatography (gradient, hexane/EtOAc=100/0 to 50/50). Appropriate fractions were concentrated.

Step 1-2: A mixture of the resulting residue (12.1 g, 22.92 mmol) in 4 M HCl in CPME (116 mL) and DCM (116 mL) was stirred at 25° C. for 2 h. The mixture was concentrated to furnish the BB42-2 (10.35 g, 22.32 mmol, hydrochloride salt) which was used in the next step without any further purification. LCMS method BB-5, t_{R}=1.35 min; [M+H]⁺=423.4.

### Step 2. N²-(((9H-fluoren-9-yl)methoxy)carbonyl)-N⁶-(4-(2-(tert-butoxy)-2-oxoethyl)piperazine-1-carbonyl)-N²-methyl-L-lysine (BB42)

BB42 was synthesized from BB42-2 analogously to the preparation of (BB41). ¹H NMR (500 MHz, DMSO-d6) δ 12.82 (br s, 1H), 7.98-7.81 (m, 2H), 7.73-7.53 (m, 2H), 7.45-7.38 (m, 2H), 7.36-7.28 (m, 2H), 6.44 (br, 1H), 4.50-4.23 (m, 4H), 3.27-3.21 (m, 4H), 3.09 (s, 2H), 3.05-2.94 (m, 2H), 2.78-2.68 (m, 3H), 2.48-2.36 (m, 4H), 1.83-1.57 (m, 2H), 1.39 (s, 9H), 1.38-1.23 (m, 2H), 1.21-1.03 (m, 2H). MS (ESI+): m/z 609.4.

### 7.2.8 Synthesis of (S)-2-((((9H-fluoren-9-yl)methoxy)carbonyl)amino)-5-((S)-2-cyano-4,4-difluoropyrrolidin-1-yl)-5-oxopentanoic acid (BB44)

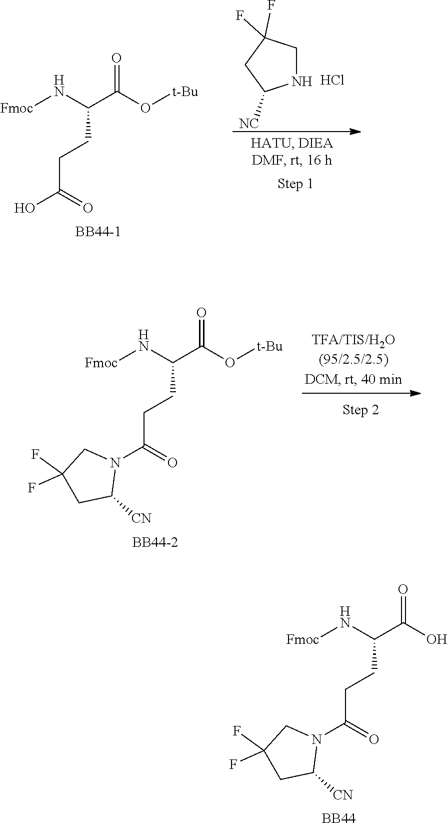

### Scheme 7.2.8. Synthesis of BB44

### Step 1. tert-butyl (S)-2-((((9H-fluoren-9-yl)methoxy)carbonyl)amino)-5-((S)-2-cyano-4,4-difluoropyrrolidin-1-yl)-5-oxopentanoate (BB44-2)

To a solution of BB44-1 (5.80 g, 13.6 mmol, 1.00 eq.) in DMF (75.0 mL) was added (S)-4,4-difluoropyrrolidine-2-carbonitrile hydrochloride (4.55 g, 27.0 mmol, 2.00 eq., CAS 869489-04-3), followed by HATU (7.83 g, 20.6 mmol, 1.51 eq.) and DIEA (9.40 mL, 52.4 mmol, 3.84 eq.). The reaction mixture was stirred at rt for 16 h. The reaction mixture was partitioned between EtOAc (50 mL) and sat. aq. NaHCO₃ (50 mL). The product was extracted with EtOAc (75 mL×2). The combined organic layers were washed with brine (100 mL×2), dried over Na₂SO₄, filtered, and concentrated to dryness in vacuo. The residue was purified by column chromatography over silica gel (heptane:EtOAc=1:0 to 1:3) to give compound BB44-2 (6.80 g, 12.5 mmol, 92% yield). ¹H NMR (400 MHz, DMSO) δ 7.90 (d, 2H), 7.72 (dd, 2H), 7.68 (d, 1H), 7.42 (t, 2H), 7.33 (t, 2H), 5.04 (dd, 1H), 4.37-4.31 (m, 1H), 4.28-4.21 (m, 2H), 4.16-4.07 (m, 1H), 4.02-3.89 (m, 2H), 2.92-2.74 (m, 2H), 2.40-2.36 (m, 2H), 1.97-1.91 (m, 1H), 1.89-1.79 (m, 1H), 1.39 (s, 9H). LC-MS method BB-1, t_{R}=6.33 min; [M−tBu+NH4]⁺=501.1.

### Step 2. (S)-2-((((9H-fluoren-9-yl)methoxy)carbonyl)amino)-5-((S)-2-cyano-4,4-difluoropyrrolidin-1-yl)-5-oxopentanoic acid (BB44)

To a solution of compound BB44-2 (3.00 g, 5.56 mmol, 1.00 eq.) in DCM (80 mL) was added a premixed solution of TFA/TIS/H₂O (95/2.5/2.5), (90 mL). The reaction mixture was stirred at rt for 40 min. The reaction mixture was diluted by addition of DCM (100 mL) then concentrated to dryness in vacuo. The residue was purified by reversed-phase column chromatography (H₂O+0.1% TFA:ACN=4:1 to 2:3) to give compound BB44 (1.65 g, 3.25 mmol, 58% yield) as a white solid. ¹H NMR (400 MHz, DMSO) δ 12.68 (s, 1H), 7.90 (d, 2H), 7.73 (dd, 2H), 7.66 (d, 1H), 7.43 (t, 2H), 7.33 (t, 2H), 5.04 (dd, 1H), 4.35-4.28 (m, 1H), 4.26-4.18 (m, 2H), 4.16-4.08 (m, 1H), 4.02-3.93 (m, 2H), 2.88-2.72 (m, 2H), 2.42-2.35 (m, 2H), 2.05-1.95 (m, 1H), 1.88-1.78 (m, 1H). LC-MS method BB-1, t_{R}=4.79 min; [M+NH₄]⁺=501.1.

### 7.2.9 Synthesis of (S)-2-((((9H-fluoren-9-yl)methoxy)carbonyl)amino)-3-(4-((4-(((tert-butoxycarbonyl)amino)methyl)-1H-1,2,3-triazol-1-yl)methyl)phenyl)propanoic acid (BB52)

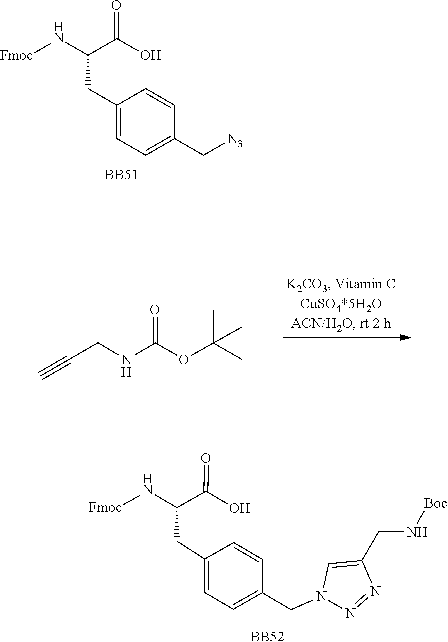

### Scheme 3. Synthesis of BB52

To a clear solution of BB51 (351.5 mg, 1 eq., 794.4 μmol, CAS 2375587-79-2) in ACN (4.0 mL) were added N-Boc-propargylamine (129.5 mg, 1.05 eq., 834.1 μmol, CAS 92136-39-5), vitamin c (209.9 mg, 1.5 eq., 1.192 mmol) and K₂CO₃ (164.7 mg, 1.5 eq., 1.192 mmol). Then a clear solution of copper(II) sulfate pentahydrate (19.84 mg, 0.1 eq., 79.44 μmol) in water (4.0 mL) was added and the resulting suspension was stirred at rt for 2 h. The reaction mixture was partitioned between EtOAc (20 mL) and 5% aq. KHSO₄ (20 mL). The organic layer was washed with brine, dried over Na₂SO₄, filtered, and concentrated to dryness in vacuo to afford BB52 (448.2 mg, 794.4 μmol, 92% yield) as a white solid. LC-MS method BB-1, t_{R}=5.55 min; [M+H]⁺=598.2.

### 7.2.10 Synthesis of (S)-2-((((9H-fluoren-9-yl)methoxy)carbonyl)amino)-3-(4-(2-(((allyloxy)carbonyl)amino)ethoxy)phenyl)propanoic acid (BB59)

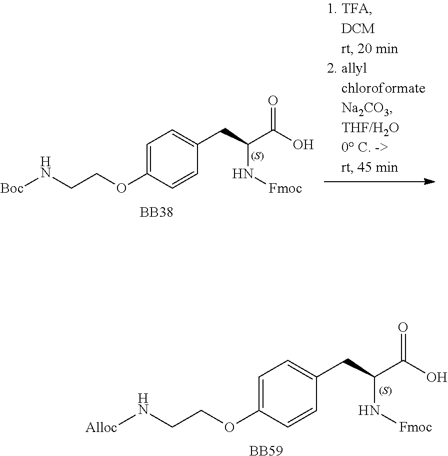

### Scheme 7.2.10. Synthesis of BB59

BB38 (1.96 g, 3.58 mmol, 1.0 eq.) was suspended in DCM (5.0 mL) and treated with TFA (7.40 g, 5.0 mL, 64.90 mmol, 18.3 eq.). The reaction mixture was stirred at rt for 20 min, then concentrated to dryness in vacuo. The residue was dissolved in THF (10.0 mL) and water (10.0 mL), then cooled to 0° C. A solution of Na₂CO₃ (969.0 mg, 9.14 mmol, 2.55 eq.) in water (5.0 mL) was added followed by allyl chloroformate (646.4 mg, 570.0 μL, 5.36 mmol, 1.50 eq.). The reaction mixture was stirred at 0° C. for 45 min. The suspension was diluted with THF (5.0 mL) and allowed to warm up to rt. The THF was removed under reduced pressure and the resulting suspension was dissolved in 10 mL of H₂O/ACN (1/1) and lyophilized. The resulting crude product was dissolved in ACN+5% TFA (5 mL) then filtered. The filtrate was concentrated under reduced pressure, then adsorbed onto Isolute® and purified by reversed-phase column chromatography (Column: RediSep Column C18 360 g; 150 mL/min; Eluent A: H₂O+0.100 TFA and eluent B: ACN). Pure fractions were combined and lyophilized to afford BB59 (1.04 g, 1.96 mmol, 55% yield) as a white solid. LCMS method 1B1-6; t_{R}=1.18 min [M+H]⁺=531.2.

### 7.3 Other Building Blocks

TABLE 7.2
Examples of other building blocks:

| No | Structure | Name | Reference or LC-MS |
|---|---|---|---|
| BB48 | 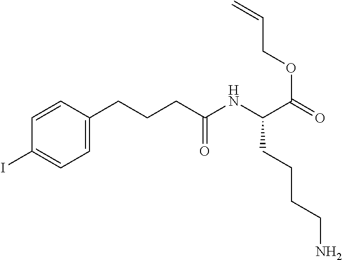 [SMILES: C=CCOC(=O)[C@H](CCCCN)NC(=O)CCCc1ccc(I)cc1] | allyl (4-(4- iodophenyl)butanoyl)- L-lysinate | Synthesized as described below, LC-MS method BB-1; t_{R} = 3.94 min; [M + H]⁺ = 459.2 |
| BB49 | 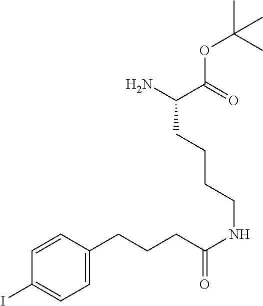 [SMILES: CC(C)(C)OC(=O)[C@@H](N)CCCCNC(=O)CCCc1ccc(I)cc1] | tert-butyl N⁶-(4-(4- iodophenyl)butanoyl)- L-lysinate | Nakashima, K., et al. (2021), Chem. Commun., 57, pp. 6432-6435. |
| BB50 | 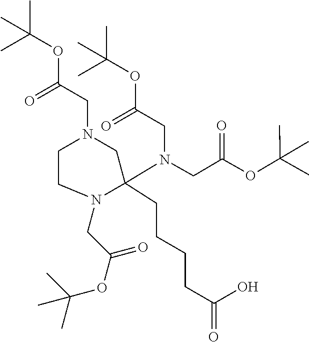 [SMILES: CC(C)(C)OC(=O)CN1CCN(CC(=O)OC(C)(C)C)C(CCCCC(=O)O)(N(CC(=O)OC(C)(C)C)CC(=O)OC(C)(C)C)C1] | 5-(6-(bis(2-(tert- butoxy)-2- oxoethyl)amino)-1,4- bis(2-(tert-butoxy)-2- oxoethyl)-1,4- diazepan-6- yl)pentanoic acid | Manzoni, L., et al. (2012), ChemMedChem, 7, pp. 1084-1093. |
| BB58 | 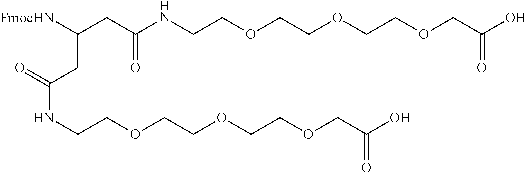 | 15-((((9H-fluoren-9- yl)methoxy)carbonyl) amino)-13,17-dioxo- 3,6,9,21,24,27- hexaoxa-12,18- diazanonacosanedioic acid | Synthesized as described below, LC-MS method P-3; t_{R} = 1.88 min [M + H]⁺ = 748.3 |

### 7.3.1 Synthesis of allyl (4-(4-iodophenyl)butanoyl)-L-lysinate (BB48)

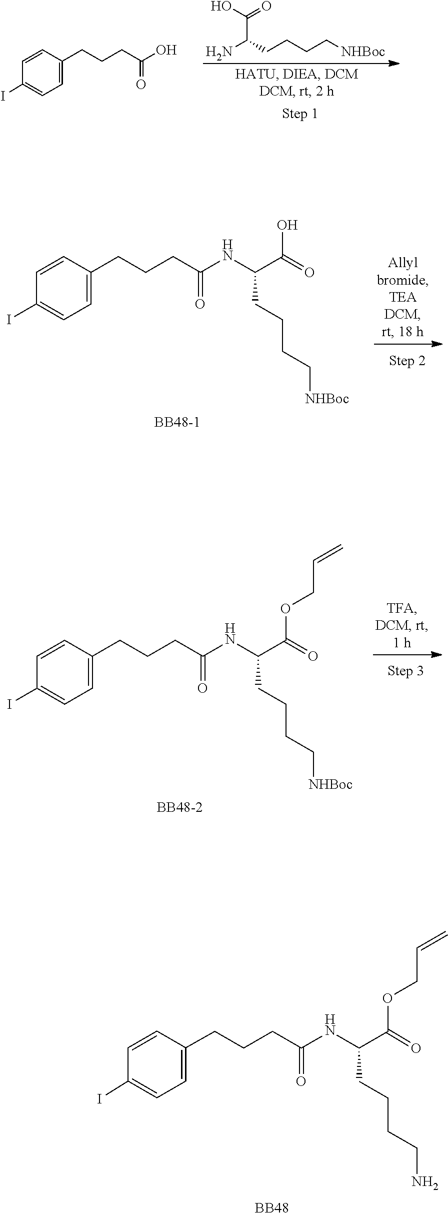

### Scheme 7.3.1. Synthesis of BB48

### Step 1. N⁶-(tert-butoxycarbonyl)-N²-(4-(4-iodophenyl)butanoyl)-L-lysine (BB48-1)

To a solution of 4-(4-iodophenyl)butanoic acid (500 mg, 1.72 mmol, 1.00 eq., CAS 27913-58-2) in DCM (17.2 mL) were added HATU (786 mg, 2.07 mmol, 1.20 eq.) and DIEA (1.80 mL, 10.3 mmol, 6.00 eq.). The resulting solution was stirred at rt for 5 min and N⁶-(tert-butoxycarbonyl)-L-lysine (764 mg, 3.10 mmol, 1.80 eq., CAS 2418-95-3) was added. The reaction mixture was stirred at rt for 2 h. The reaction mixture was diluted with DCM (100.0 mL), washed with 0.1 M aq. HCl (50.0 mL), dried over Na₂SO₄, filtered, and concentrated to dryness in vacuo. LC-MS method BB-6, t_{R}=1.23 min; [M+H]⁺=519.3.

### Step 2. Allyl N⁶-(tert-butoxycarbonyl)-N²-(4-(4-iodophenyl)butanoyl)-L-lysinate (BB48-2)

Crude compound BB48-1 was dissolved in DCM (17.2 mL) under N₂. To this solution were added simultaneously allyl bromide (0.27 mL, 3.45 mmol, 2.00 eq., CAS 106-95-6) and triethylamine (1.20 mL, 8.62 mmol, 5.00 eq.). The resulting solution was stirred at rt for 1 h. To achieve full conversion, a second addition of allyl bromide (1.19 mL, 13.79 mmol, 8.00 eq., CAS 106-95-6) was performed. The solution was stirred at rt for 17 h. The reaction mixture was diluted with DCM (50.0 mL), washed with sat. NaHCO₃ (50.0 mL), dried over Na₂SO₄, filtered, and concentrated to dryness in vacuo. LC-MS method BB-6, t_{R}=1.41 min; [M+H]⁺=559.2.

### Step 3. Allyl (4-(4-iodophenyl)butanoyl)-L-lysinate (BB48)

Crude compound BB48-2 was dissolved in DCM (17.2 mL), to which was added TFA (1.32 mL, 17.24 mmol, 10.00 eq.). The resulting solution was stirred at rt for 1 h. The reaction mixture was then concentrated to dryness in vacuo. The residue was purified by column chromatography over silica gel (DCM:DCM/MeOH (8/2 with 10% NH₄OH)=1:0 to 0:0.9) to afford compound BB48 (567 mg, 1.20 mmol, 70% yield over 3 steps). ¹H NMR (400 MHz, DMSO) δ 8.22 (d, 1H), 7.71 (br s, 2H), 7.63 (m, 2H), 7.01 (m, 2H), 5.98-5.79 (m, 1H), 5.30 (dd, 1H), 5.22-5.17 (m, 1H), 4.57 (br d, 2H), 4.28-4.19 (m, 1H), 2.81-2.70 (m, 2H), 2.54-2.53 (m, 1H), 2.12 (t, 2H), 1.80-1.73 (m, 2H), m 1.74-1.56 (m, 2H), 1.63-1.48 (m, 2H), 1.42-1.26 (m, 2H). LC-MS method BB-1, t_{R}=3.94 min; [M+H]⁺=459.2.

### 7.3.2 Synthesis of bis(perfluorophenyl) 3-((((9H-fluoren-9-yl)methoxy)carbonyl)amino)pentanedioate (BB53)

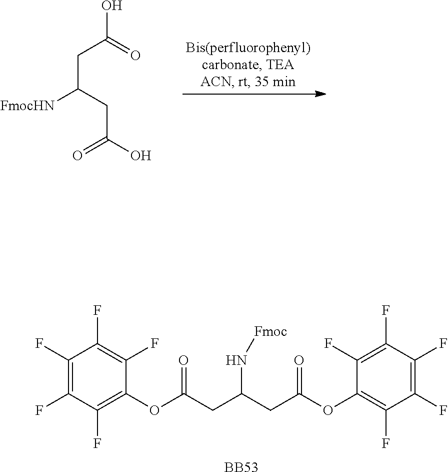

### Scheme 7.3.2. Synthesis of BB53

3-((((9H-fluoren-9-yl)methoxy)carbonyl)amino)pentanedioic acid (175.0 mg, 473.8 μmol, 1.0 eq.) was suspended in ACN (10.0 mL) and treated with TEA (63.8 mg, 87.8 μL, 630.1 μmol, 1.33 eq.), followed by addition of bis(perfluorophenyl) carbonate (466.8 mg, 1.184 mmol, 2.5 eq.). The reaction mixture was stirred at rt for 35 min. The reaction mixture was concentrated to dryness in vacuo and the product was isolated by preparative HPLC (Column: SunFire C18 OBD, 30×100 mm, 5 μm; 40 mL/min; Eluent A: H₂O+0.1% TFA and eluent B: ACN). Pure fractions were combined and lyophilized to afford BB53 (232.0 mg, 331 μmol, 70% yield) as a white solid. LCMS method P-3 t_{R}=3.48 min, [M+Na]⁺=724.1.

### 7.3.3 Synthesis of 15-((((9H-fluoren-9-yl)methoxy)carbonyl)amino)-13,17-dioxo-3,6,9,21,24,27-hexaoxa-12,18-diazanonacosanedioic acid (BB58)

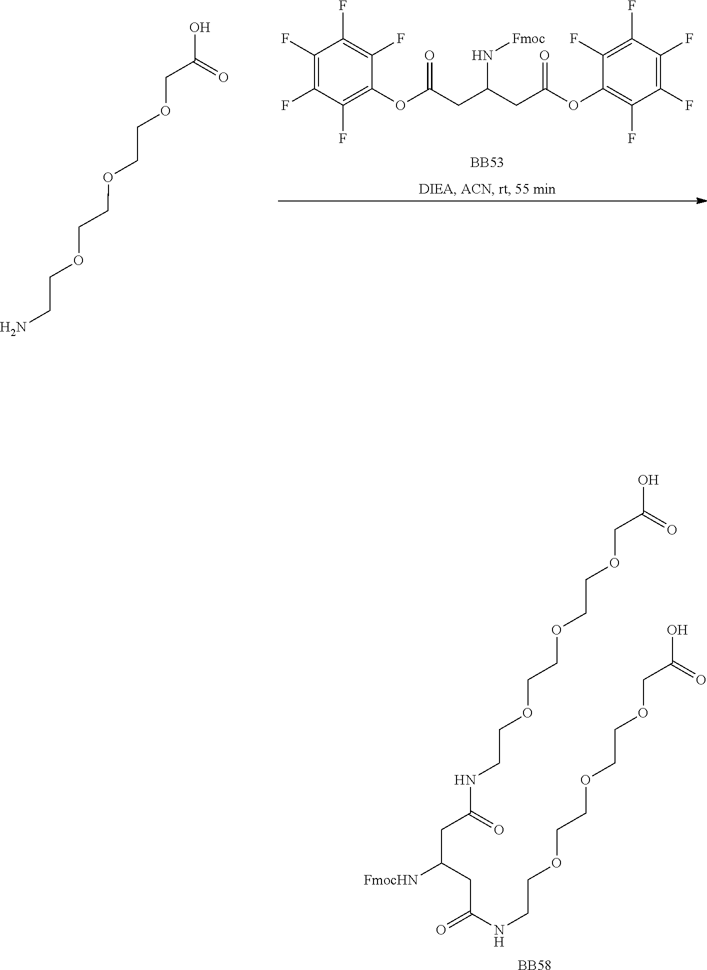

### Scheme 7.3.3. Synthesis of BB58

2-(2-(2-(2-aminoethoxy)ethoxy)ethoxy)acetic acid (69.2 mg, 334.0 μmol, 2.2 eq.) was suspended in ACN (5.0 mL) and treated with DIEA (372.8 mg, 502 μL, 2.885 mmol, 19.0 eq.), followed by portion-wise addition of BB53 (106.5 mg, 151.8 μmol, 1.0 eq.). The reaction mixture was stirred at rt for 55 min. The reaction mixture was concentrated to dryness in vacuo and the product was isolated by preparative HPLC (Column:SunFire C18 OBD, 30×100 mm, 5 μm; 40 mL/min; Eluent A: H₂O+0.1% TFA and eluent B: ACN). Pure fractions were combined and lyophilized to afford BB58 (64.0 mg, 85.6 μmol, 56% yield) as a white solid. LCMS method P-3 t_{R}=1.88 min, [M+H]⁺=748.3.

### Example I. Analysis of Peptide Affinity and Potency Against FAP and Selectivity Against DPP4

### I. Surface Plasmon Resonance (SPR) for Assessment of Peptide Affinity

Peptide affinities (K_{D}) were determined by SPR using a Biacore™ 8K device (Cytiva) towards the following proteins: biotinylated His, Avi tag, human FAP (26-760, FAP-H82Q6), biotinylated His, Avi tag, mouse FAP (26-761, FAP-M82Q5), His tag, human DPP4 (34-766, DP4-H82E3), all purchased from AcroBiosystems and His tag, mouse DPP4 (29-760, 954-SE) from R&D Systems. The FAP proteins and their homologous were diluted to a concentration of 2 μg/mL into acetate buffer pH 5.5, then immobilized onto a CM5 sensorchip (Cytiva, BR-1005-30) to reach a response around 2000 R U. The running buffer HBS-EP+pH 7.6 (20× from Teknova CAT. No: H8022) contained 10 mM HEPES pH 7.6, 150 mM NaCl, 3 mM EDTA, 0.05% Tween20 and 2% DMSO. Experiments were carried out at 25° C. using a flow rate of 30 μL/min. Compounds were tested in single cycle kinetic mode at 8 different concentrations. Curve fitting was performed using the Biacore™ 8K evaluation software. The sensorgrams were fitted by applying a 1:1 binding model to calculate kinetic rate constants and equilibrium dissociation constants (K_{D}).

When peptides reached the nM range of affinity and exhibited slow dissociation rates, the oligo biotin capture method was preferred for human and mouse FAP SPR. For this assay, the setup had the same conditions (flow rate, running buffer, peptide dilution) as described above for the CM5 method. The oligo CAP reagent obtained in the CAPture Kit (Ref. 28920234) was 1/5 diluted into running buffer and used with a contact time of 300 s at a flow rate of 2 μL/min. The human and mouse FAP constructs described above were used (from AcroBiosystems or produced in house). The FAP proteins were diluted to a concentration of 2 μg/mL into acetate pH 5.5, 0.05% Tween20 and used with a contact time of 600 s at a flow rate of 10 μL/min. The peptides were tested with an association time of 280 s followed by a dissociation time of 3000 s at a flow rate of 30 μL/min. The regeneration was done using the regeneration solution (8 M guanidine-HCl, 1 M NaOH) as obtained with a contact time of 270 s at a flow rate of 10 μL/min. The data resulting from these studies for the compounds disclosed herein are presented in Table 7.3. These results indicate that the compounds disclosed herein have high affinity for FAP. The compounds marked with an asterisk were tested against human and mouse DDP4. All of them displayed a K_{D} above 6 M, indicating a high selectivity of the compounds towards DPP4.

### II. Enzymatic FAP Competition Assay for Assessment of Peptide Potency

Peptide potencies (IC₅₀) to inhibit the FAP enzymatic activity were determined using a fluorescence-based assay towards the biotinylated His, Avi tag, human FAP (26-760, FAP-H82Q6) and the biotinylated His, Avi tag, mouse FAP (26-761, FAP-M82Q5). Standard assay conditions consisted of 20 μL total volume in white 384-well plates (Greiner, Ref. 784075), in 50 mM phosphate buffer pH 7.5 containing 150 mM NaCl, 0.05% Tween20 and 1% final DMSO. Tested peptides at 14 different concentrations up to 1 μM were added to 0.01 nM human or mouse FAP. The peptides were incubated for 24 hours together with the protein before adding the substrate 50 μM Z-Gly-Pro-AMC (I-1145 from Bachem). Samples were then incubated at 22-24° C. for 4 h before reading the fluorescence at excitation wavelength of 380 nm and emission wavelength of 460 nm using the Tecan Infinite® M1000 PRO. The high control (100% activity) was not containing any peptide, and an internal potent peptide was added at 10 μM concentration for the low control (100% inhibition). IC₅₀ values were calculated by curve fitting using an in-house developed software (Novartis Helios software application) using the method described by Fomenko et al., 2006 (regression algorithms for nonlinear dose-response curve fitting). Following normalization of activity values for the wells to % inhibition (% inhibition=[(high control-sample)/(high control-low control)]x 100). IC₅₀ fitting was carried out from the duplicated points. The data resulting from these studies for the compounds disclosed herein are presented in Table 7.3. These results indicate the compounds disclosed herein bind to human and mouse FAP and inhibit human and mouse FAP enzymatic activity which confirms and correlates well with the binding affinity obtained by SPR.

TABLE 7.3
Summary of biological activities, in vitro, as tested for
certain compounds of the disclosure.

| | | | h. | m. |
|---|---|---|---|---|
| | | | FAP, | FAP, |
| | h. FAP, | m. FAP, | Enz., | Enz., |
| Example | SPR, K_{D} | SPR, K_{D} | IC₅₀ | IC₅₀ |
| No. | (nM) | (nM) | (nM) | (nM) |
| A1 | <0.007 | <0.018 | 0.009 | 0.028 |
| A2 | <0.011 | <0.023 | 0.012 | 0.047 |
| A3 | | 1.9 | 0.76 | 0.95 |
| A4 | | 0.47 | 0.203 | 0.57 |
| A5 | | 0.62 | 0.07 | 0.5 |
| A6 | | 0.188 | 0.071 | 0.178 |
| A13 | <0.005 | 0.011 | 0.005 | 0.011 |
| A14 | | 0.239 | 0.092 | 0.37 |
| A18 | | 0.222 | 0.27 | 0.71 |
| A19 | | 0.26 | 0.145 | 0.66 |
| A20 | | 8.7 | 2.6 | 8.7 |
| A21 | | 15 | 5.7 | 14 |
| A22 | | 0.87 | 0.33 | 0.67 |
| A23 | | 0.45 | 0.111 | 0.48 |
| A24 | | 0.85 | 0.37 | 1.3 |
| A25 | | 0.47 | 0.209 | 0.86 |
| A26 | | 2.7 | 0.47 | 1.6 |
| A27 | | 0.219 | 0.045 | 0.138 |
| A28 | | 0.52 | 0.063 | 0.33 |
| A29 | | 0.044 | 0.059 | 0.082 |
| A30 | | 2.4 | 0.79 | 2.5 |
| A31 | | 0.106 | 0.040 | 0.158 |
| A32 | 0.27 | 0.55 | 0.239 | 1.2 |
| A33 | | 1.1 | 1.0 | 3.2 |
| A34 | | 0.25 | 0.055 | 0.40 |
| A41 | | 12 | 2.2 | 5.3 |
| A42 | | 0.200 | 0.089 | 0.36 |
| A43 | | 0.188 | 0.069 | 0.189 |
| A44 | | 2.7 | 0.54 | 2.5 |
| A45 | | 0.54 | 0.192 | 0.93 |
| A46 | | 2.2 | 0.50 | 2.8 |
| A47 | | 0.48 | 0.181 | 0.60 |
| A50 | | 2.8 | 0.95 | 2.4 |
| A51 | 0.163 | 0.185 | 0.117 | 0.40 |
| A52 | 0.118 | 0.36 | 0.104 | 0.737 |
| A53 | | 0.72 | 0.40 | 1.3 |
| A55 | | 135 | 24 | 84 |
| A56 | | 32 | 4.2 | 31 |
| A57 | | 19 | 6.6 | 25 |
| A58 | | 75 | 19 | 46 |
| A59 | | 0.38 | 0.136 | 0.49 |
| A60 | | 0.05 | 0.041 | 0.127 |
| A61 | | 0.162 | 0.065 | 0.30 |
| A62 | | 0.32 | 0.063 | 0.31 |
| A63 | | 20 | 15 | 55 |
| A64 | | 3.6 | 2.4 | 4.1 |
| A65 | | 1.8 | 0.34 | 2.8 |
| A66 | | 0.46 | 0.27 | 1.2 |
| A67 | | 208 | 76 | 252 |
| A68 | | 35 | 12 | 80 |
| A69 | | 0.222 | 0.121 | 0.47 |
| A70 | | 0.104 | 0.036 | 0.116 |
| A71 | | 0.242 | 0.69 | 0.61 |
| A72 | | >6000 | 237 | 1000 |
| A73 | | 0.081 | 0.028 | 0.101 |
| A74 | | 4.6 | 1.4 | 3.3 |
| B1 | <0.011 | <0.018 | 0.005 | 0.013 |
| B2 | <0.013 | <0.022 | 0.007 | 0.018 |
| B3 | | 0.093 | 0.025 | 0.091 |
| B16 | | 0.010 | 0.009 | 0.030 |
| B17 | | 0.133 | 0.033 | 0.158 |
| B23 | | 0.077 | 0.012 | 0.059 |
| B24 | | 0.066 | 0.012 | 0.108 |
| B25 | | 0.44 | 0.100 | 0.38 |
| B26 | | 0.105 | 0.027 | 0.117 |
| B30 | | 0.119 | 0.019 | 0.118 |
| B31 | | 0.25 | 0.030 | 0.100 |
| B33 | | <0.034 | 0.015 | 0.039 |
| B34 | | <0.034 | 0.007 | 0.017 |
| B35 | | 0.059 | 0.021 | 0.055 |
| B37 | | <0.022 | 0.017 | 0.042 |
| B38 | | <0.076 | 0.031 | 0.073 |
| B40 | | <0.024 | 0.006 | 0.012 |
| C1* | 0.006 | <0.060 | 0.006 | 0.016 |
| C1* | <0.010** | <0.016** | 0.006 | 0.016 |
| C2* | <0.008 | <0.036 | 0.009 | 0.023 |
| C3 | | <0.041 | 0.007 | 0.019 |
| C4 | | <0.039 | 0.011 | 0.032 |
| C5 | | <0.084 | 0.019 | 0.049 |
| C6 | | <0.035 | 0.024 | 0.049 |
| C7 | | <0.068 | 0.014 | 0.034 |
| C8* | 0.004 | 0.006 | 0.007 | 0.012 |
| C9 | | 0.032 | 0.010 | 0.024 |
| C10 | | 0.033 | 0.010 | 0.024 |
| C11 | | 0.028 | 0.009 | 0.024 |
| C12* | 0.009 | <0.009 | 0.006 | 0.014 |
| C13* | <0.011 | 0.024 | 0.004 | 0.011 |
| C14 | | 0.048 | 0.009 | 0.024 |
| C15 | | 0.013 | 0.003 | 0.009 |
| C16 | | 0.40 | 0.084 | 0.29 |
| C17 | | <0.013 | 0.005 | 0.010 |
| C18 | | 0.011 | 0.008 | 0.012 |
| C19* | <0.018 | <0.040 | 0.018 | 0.031 |
| C20 | | <0.031 | 0.016 | 0.030 |
| C21 | | <0.029 | 0.007 | 0.016 |
| C22* | 0.41 | 0.64 | 0.43 | 1.1 |
| C23* | <0.009 | <0.011 | 0.016 | 0.061 |
| C23* | <0.009 | <0.060** | 0.016 | 0.061 |
| C24 | | 0.035 | 0.013 | 0.059 |
| C25 | | 0.34 | 0.098 | 0.35 |
| C26 | | 0.138 | 0.018 | 0.071 |
| C27* | | 0.102 | 0.026 | 0.072 |
| C28 | | 0.024 | 0.023 | 0.041 |
| C29 | | <0.018 | 0.011 | 0.024 |
| C30 | | <0.017 | 0.034 | 0.193 |
| C31 | | <0.010 | 0.007 | 0.027 |
| C32 | | <0.027 | 0.017 | 0.044 |
| C33 | | <0.02 | 0.012 | 0.026 |
| C34 | | 0.038 | 0.008 | 0.019 |
| C35 | | <0.065 | 0.020 | 0.049 |
| C36 | | <0.021 | 0.020 | 0.042 |
| C37 | | <0.047 | 0.017 | 0.029 |
| C38 | | 0.037 | 0.012 | 0.048 |
| C39 | | <0.024 | 0.015 | 0.051 |
| C40 | | <0.02 | 0.006 | 0.019 |
| C41 | | <0.043 | 0.013 | 0.031 |
| C42 | | <0.015 | 0.007 | 0.012 |
| C43 | | <0.04 | 0.011 | 0.034 |
| C44* | <0.15 | <0.023 | 0.006 | 0.019 |
| C44* | <0.015** | <0.023 | 0.006 | 0.019 |
| C45 | | <0.023 | 0.019 | 0.060 |
| C46 | | <0.016 | 0.012 | 0.032 |
| C47 | | <0.028 | 0.008 | 0.025 |
| C48 | | <0.019 | 0.008 | 0.029 |
| C49 | | <0.012 | 0.005 | 0.016 |
| C50 | | <0.020 | 0.046 | 0.052 |
| C51 | | 0.026 | 0.030 | 0.058 |
| C52* | <0.015 | <0.022 | 0.009 | 0.026 |
| C53 | | <0.035 | 0.013 | 0.035 |
| C54 | | <0.016 | 0.004 | 0.012 |
| C55 | | <0.014 | 0.006 | 0.022 |
| C56* | <0.007 | <0.023 | 0.009 | 0.037 |
| C57* | <0.013 | <0.014 | 0.006 | 0.016 |
| C58* | <0.007 | <0.017 | 0.006 | 0.017 |
| D1 | | <0.03 | | |
| D2 | | <0.040 | | |
| F1* | | <0.027 | 0.008 | 0.032 |
| F2 | | <0.044 | 0.013 | 0.040 |
| M1* | <0.030 | <0.057 | 0.039 | 0.134 |
| M2* | <0.030 | <0.060 | 0.026 | 0.095 |
| M3* | <0.043 | <0.078 | 0.035 | 0.144 |
| M4* | 0.32 | 0.51 | 0.38 | 1.1 |
| M5* | 0.137 | 0.25 | 0.089 | 0.71 |

The compounds with an asterisk (*) were tested towards human and mouse DPP4 and showed no activity up to 6 μM. Values marked with an asterisk (**) are values including further experimental repetition.

### Example II. Biological Data

### In Vivo Biodistribution Experiments

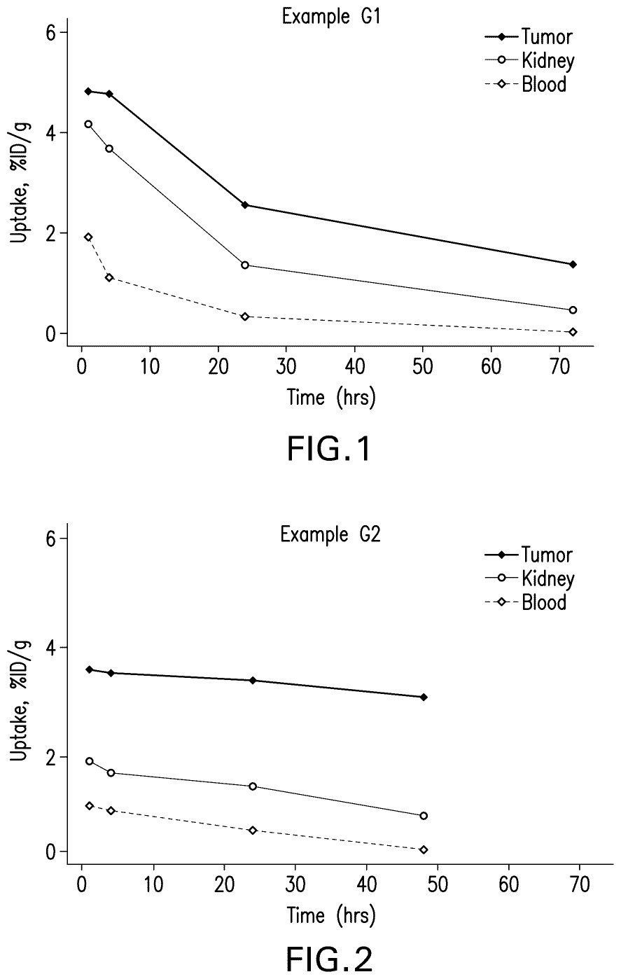

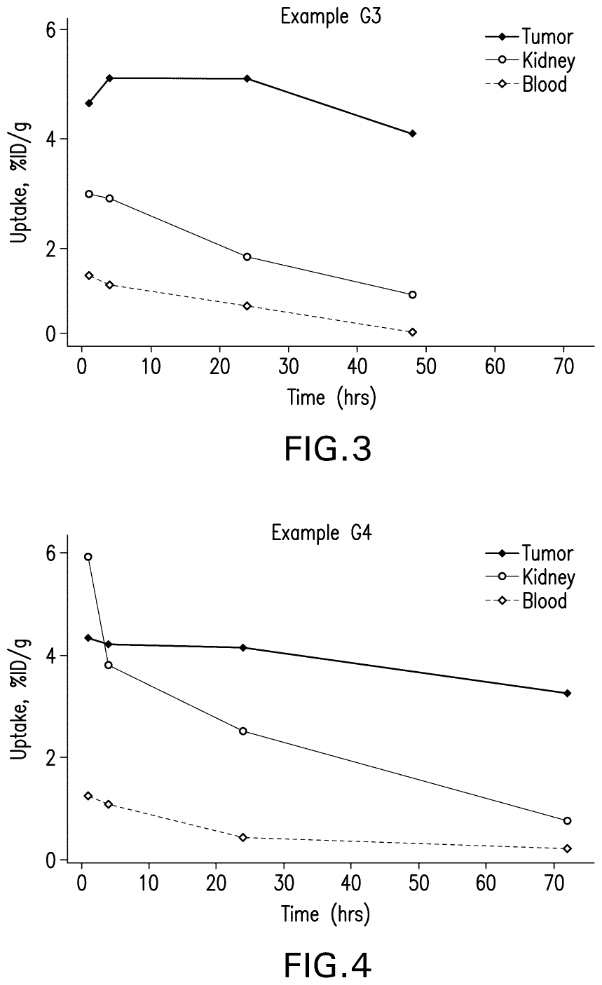

Biodistribution studies were performed at Minerva Imaging. All animal experimentation was carried out under a license approved by the National Animal Experiments Inspectorate under the Ministry of Environment and Food of Denmark, and further approved by the Novartis Animal Welfare Office. Biodistribution studies were performed in Nu(NCr)-Foxn1nu-homozygous mice bearing subcutaneous Capan-2 tumors. Mice were subcutaneously implanted with 10×10⁶ Capan-2 cells into right flank. The biodistribution studies were typically performed when tumors were approximately 100-200 mm³ in volume. Compounds according to Examples G1, G2, G3, and G4 (see Table 6.1) were radiolabeled as described in the radiochemistry part (section 6.3). Animals were single dose administered with an activity concentration of 4 MBq/nmol per animal. The biodistribution of the compounds was assessed using conventional ex vivo biodistribution at different time points, 1, 4, 24, 48 and 72 hrs after intravenous injection of Lu-177 labelled compounds. Mice were euthanized and organs were collected, weighed, and placed inside counting vials. Organ uptake was assessed by gamma counting using an automated gamma counter for 60 seconds immediately after tissue collection. Tissue counts and injected doses for individual animals were decay-corrected to the time of injection. Three animals were used per time point. Results expressed as a percentage of injected dose per gram of the tissue (% ID/g). The results of the biodistribution studies (tumor, kidney and blood uptake) are presented in FIGS. 1-4.

### In Vivo Antitumor Efficacy Experiments

Efficacy studies were performed at Minerva Imaging. All animal experimentation was carried out under a license approved by the National Animal Experiments Inspectorate under the Ministry of Environment and Food of Denmark, and further approved by the Novartis Animal Welfare Office.

### Capan 2:

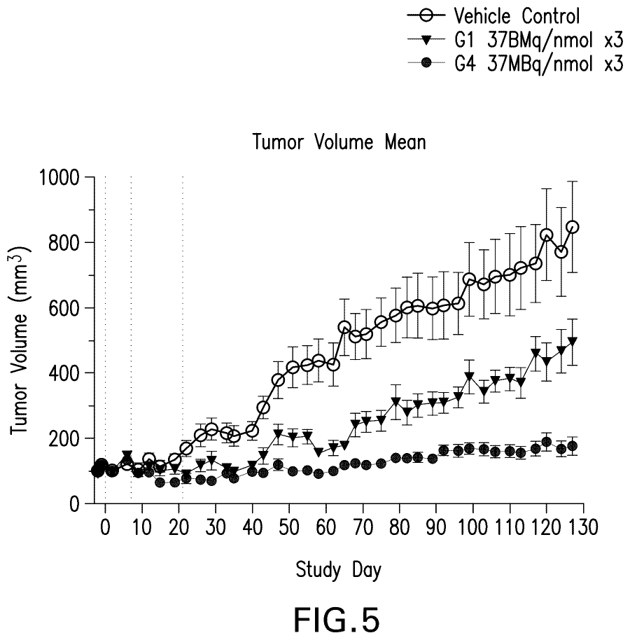

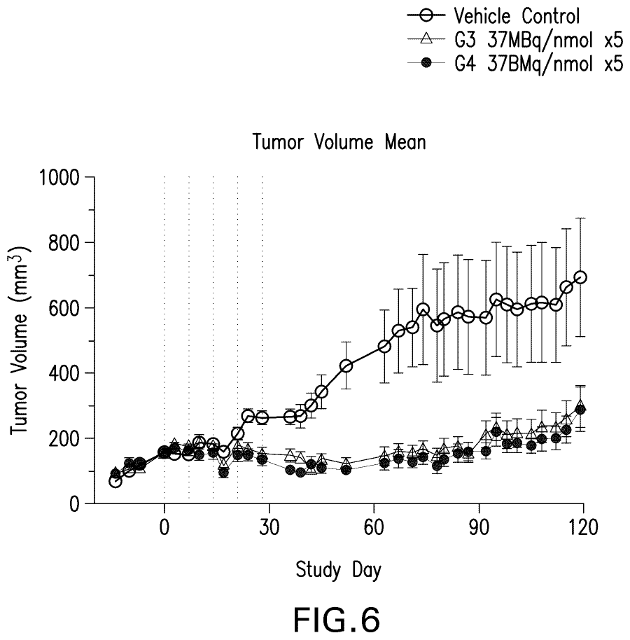

Antitumor efficacy studies were performed in Nu(NCr)-Foxn1nu-homozygous mice bearing subcutaneous Capan-2 tumors. Mice were subcutaneously implanted with 10×10⁶ Capan-2 cells into the right flank. The studies were typically performed when tumors were approximately 100-200 mm³ in volume. Compounds according to Examples G1, G2, G3, and G4 (see Table 6.1) were radiolabeled as described in the radiochemistry part (section 6.3). Animals were administered with an activity concentration of 37 MBq/nmol per animal on the weeks indicated by the dotted line. The efficacy was evaluated by tumor growth monitoring, shown in FIGS. 5 and 6.

### ST4454:

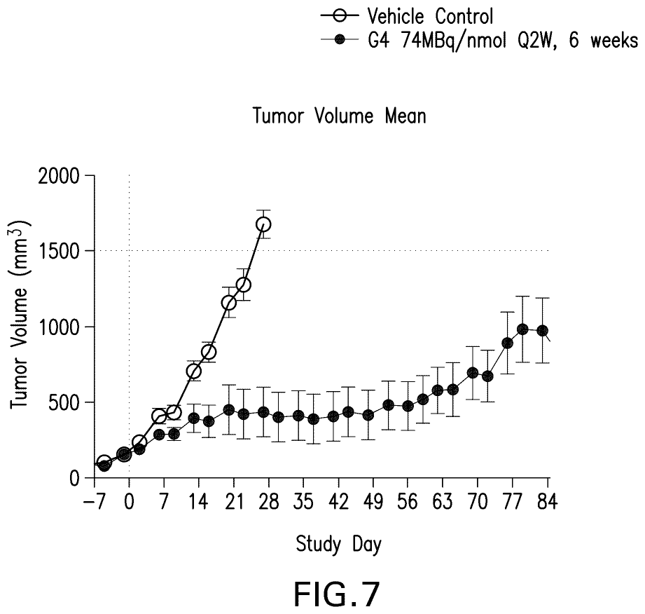

Antitumor efficacy studies were performed in NMRI mice bearing a subcutaneous ST4454 PDX. Mice were subcutaneously implanted with a PDX fragment into the right flank. The studies were typically performed when tumors were approximately 100-200 mm³ in volume. Example G4 (see Table 6.1) was radiolabeled as described in the radiochemistry part (section 6.3). Animals were administered with an activity concentration of 74 MBq/nmol per animal every second week for a total of six weeks. The efficacy was evaluated by tumor growth monitoring, as shown in FIG. 7.

### Example III. Table of Compounds

| Ex. No. | SEQ ID NO. | A¹ | A² | A³ | A⁴ | A⁵ | A⁶ | A⁷ | A⁸ | A⁹ | A¹⁰ |
|---|---|---|---|---|---|---|---|---|---|---|---|
| A1 | 1 | Gly | Gly | NMePhe | F(4amPEG2NH2) | Aoc | Trp | Gly | K(COpipzaa) | Asp | D-Pro |
| A2 | 2 | Gly | Gly | NMePhe | F(4amPEG2NH2) | hS(Pr) | Trp | Gly | K(COpipzaa) | Asp | D-Pro |
| A3 | 3 | Gly | Gly | NMePhe | Tyr | Ahp | Trp | Gly | MeK(COPipzaa) | Asp | D-Pro |
| A4 | 4 | Gly | Gly | NMePhe | Tyr | Ahp | Trp | Gly | Orn(COPipzaa) | Asp | D-Pro |
| A5 | 5 | Gly | Gly | NMePhe | Tyr | Ahp | Trp | Gly | Dab(COPipzaa) | Asp | D-Pro |
| A6 | 6 | Gly | Gly | NMePhe | F(4OEtNH2) | Ahp | Trp | Gly | K(COpipzaa) | Asp | D-Pro |
| A7 | 7 | Gly | Gly | NMe3PyA | F(4amPEG2NH2) | hS(Pr) | W7N | Gly | K(COpipzaa) | Asp | D-Pro |
| A8 | 8 | Glu | Gly | NMePhe | F(4amPeG2NH2) | hS(Pr) | Trp | Gly | K(COpipzaa) | Asp | D-Pro |
| A9 | 9 | Glu | Gly | NMe3PyA | F(4amPEG2NH2) | hS(Pr) | W7N | Gly | K(COpipzaa) | Asp | D-Pro |
| A10 | 10 | hSer | Gly | NMePhe | F(4amPEG2NH2) | hS(Pr) | Trp | Gly | K(COpipzaa) | Asp | D-Pro |
| A11 | 11 | hSer | Gly | NMe3PyA | F(4amPEG2NH2) | hS(Pr) | W7N | Gly | K(COpipzaa) | Asp | D-Pro |
| A12 | 12 | Gly | Gly | NMePhe | F(4am) | Ahp | Trp | Gly | Tyr | Asp | D-Pro |
| A13 | 13 | Gly | Gly | NMePhe | Tyr | Ahp | Trp | Gly | F(4OMe) | Asp | D-Pro |
| A14 | 14 | Gly | Gly | NMePhe | Tyr | Ahp | Trp | Gly | F(3OH) | Asp | D-Pro |
| A15 | 15 | Pro | Gly | NMePhe | F(4amPEG2NH2) | Aoc | Trp | Gly | K(COpipzaa) | Asp | D-Pro |
| A16 | 16 | Gly | Gly | NMePhe | F(4amPEG2NH2) | Ahp | Trp | Gly | K(COpipzaa) | Asp | D-Pro |
| A17 | 17 | Gly | Gly | NMePhe | F4(MeTriazolylMeNH2) | Ahp | Trp | Gly | Tyr | Asp | D-Pro |
| A18 | 18 | Asp | Gly | NMePhe | Tyr | Ahp | Trp | Gly | Tyr | Asp | D-Pro |
| A19 | 19 | Ser | Gly | NMePhe | Tyr | Ahp | Trp | Gly | Tyr | Asp | D-Pro |
| A20 | 20 | Lys | Glu | Phe | Tyr | Ahp | Trp | Gly | Tyr | Asp | D-Pro |
| A21 | 21 | Lys | Glu | NMePhe | Tyr | Ahp | Trp | Gly | Tyr | Asp | D-Pro |
| A22 | 22 | Lys | Gly | Phe | Tyr | Ahp | Trp | Gly | Tyr | Asp | D-Pro |
| A23 | 23 | Lys | Gly | NMePhe | Tyr | Ahp | W(7F) | Gly | Tyr | Asp | D-Pro |
| A24 | 24 | Lys | Gly | NMePhe | Tyr | Ahp | W(6OH) | Gly | Tyr | Asp | D-Pro |
| A25 | 25 | Lys | Gly | NMePhe | Tyr | Ahp | W(7Cl) | Gly | Tyr | Asp | D-Pro |
| A26 | 26 | Lys | Gly | NMePhe | Tyr | Ahp | W7N | Gly | Tyr | Asp | D-Pro |
| A27 | 27 | Gly | Gly | NMePhe | F(4amPEG2NH2) | Ahp | Trp | Gly | Tyr | Asp | D-Pro |
| A28 | 28 | Gly | Gly | NMePhe | F(4AmAc) | Ahp | Trp | Gly | Tyr | Asp | D-Pro |
| A29 | 29 | Lys | Gly | NMePhe | Tyr | Q(Pyrro(2CN4F2)) | Trp | Gly | Tyr | Asp | D-Pro |
| A30 | 30 | Lys | Gly | NMePhe | Tyr | Q(Pyrro(4F2)) | Trp | Gly | Tyr | Asp | D-Pro |
| A31 | 31 | Lys | Gly | NMePhe | Tyr | Aoc | Trp | Gly | Tyr | Asp | D-Pro |
| A32 | 32 | Pro | Gly | NMePhe | F(4OEtNH2) | Ahp | Trp | Gly | Tyr | Asp | D-Pro |
| A33 | 33 | trans4Hyp | Gly | NMePhe | F(4OEtNH2) | Ahp | Trp | Gly | Tyr | Asp | D-Pro |
| A34 | 34 | Gly | Gly | NMePhe | Tyr | Aoc(3R-OH) | Trp | Gly | Tyr | Asp | D-Pro |
| A35 | 35 | Gly | gly | NMePhe | F(4amPEG2NH2) | Aoc | Trp | Gly | F(4OMe) | Asp | D-Pro |
| A36 | 36 | Gly | Gly | NMePhe | F(4amPEG2NH2) | Aoc | Trp | Gly | F(3OH) | Asp | D-Pro |
| A37 | 37 | Gly | Gly | NMePhe | F(4amPEG2NH2) | Q(Pyrro(2CN4F2)) | Trp | Gly | K(COpipzaa) | Asp | D-Pro |
| A38 | 38 | Pro | Gly | NMePhe | F(4am) | hS(Pr) | Trp | Gly | K(COpipzaa) | Asp | D-Pro |
| A39 | 39 | Pro | Gly | NMePhe | F(4amPEG2NH2) | hS(Pr) | Trp | Gly | K(COpipzaa) | Asp | D-Pro |
| A40 | 40 | Gly | Gly | NMePhe | F(4amPEG2NH2) | hS(Pr) | W7N | Gly | K(COpipzaa) | Asp | D-Pro |
| A41 | 41 | Lys | Gly | NMePhe | Tyr | Ahp | Trp | Gly | Tyr | Asp | D-trans4hyp |
| A42 | 42 | hSer | Gly | NMePhe | Tyr | Ahp | Trp | Gly | Tyr | Asp | D-Pro |
| A43 | 43 | Lys | Gly | NMecHexA | Tyr | Ahp | Trp | Gly | Tyr | Asp | D-Pro |
| A44 | 44 | Lys | Gly | NMecPenA | Tyr | Ahp | Trp | Gly | Tyr | Asp | D-Pro |
| A45 | 45 | Gly | Gly | NMePhe | F(4OEtNH2) | Ahp | Trp | Gly | 3PyA(6NH2) | Asp | D-Pro |
| A46 | 46 | K(COpipzaa) | Gly | NMePhe | F(4OEtNH2) | Ahp | Trp | Gly | Tyr | Asp | D-Pro |
| A47 | 47 | hSer | Gly | NMePhe | F(4OEtNH2) | Ahp | Trp | Gly | Tyr | Asp | D-Pro |
| A48 | 48 | Gly | Gly | NMePhe | F(4amPEG2NH2) | Aoc | W7N | Gly | K(COpipzaa) | Asp | D-Pro |
| A49 | 49 | Gly | Gly | NMe3PyA | F(4amPEG2NH2) | Aoc | Trp | Gly | K(COpipzaa) | Asp | D-Pro |
| A50 | 50 | Gly | Gly | NMePhe | Tyr | Ahp | Trp | Gly | NMeY | Asp | D-Pro |
| A51 | 51 | Pro | Gly | NMePhe | Tyr | Ahp | Trp | Gly | Tyr | Asp | D-Pro |
| A52 | 52 | Gly | Gly | NMePhe | Tyr | Ahp | Trp | Gly | Tyr | Asp | D-Pro |
| A53 | 53 | Lys | Gly | NMePhe | Tyr | Ahp | Trp | Gly | Tyr | Asp | D-Pro |
| A54 | 54 | Gly | Gly | NMePhe | Tyr | Ahp | Trp | Gly | Tyr | Lys | D-Pro |
| A55 | 55 | Gly | Gly | NMePhe | Tyr | Ahp | Trp | Gly | Tyr | K(Ac) | D-Pro |
| A56 | 56 | Gly | Gly | NMePhe | Tyr | Ahp | Trp | Gly | Tyr | Ala | D-Pro |
| A57 | 57 | Gly | Gly | NMePhe | Tyr | Ahp | Trp | Gly | Tyr | hSer | D-Pro |
| A58 | 58 | Gly | Gly | NMePhe | Tyr | Ahp | Trp | Gly | Tyr | Asn | D-Pro |
| A59 | 59 | Gly | Gly | NMePhe | F(4OEtNH2) | Ahp | Trp | Gly | Tyr | Asp | D-Pro |
| A60 | 60 | Gly | Gly | cBuA | Tyr | Ahp | Trp | Gly | Tyr | Asp | D-Pro |
| A61 | 61 | Gly | Gly | NMePhe | Tyr | Ahp | W(7OMe) | Gly | Tyr | Asp | D-Pro |
| A62 | 62 | Gly | Gly | NMecBuA | Tyr | Ahp | Trp | Gly | Tyr | Asp | D-Pro |
| A63 | 63 | Gly | Gly | NMePhe | Tyr | Ahp | Trp | Gly | Tyr | Asp | Mor(3R-COOH) |
| A64 | 64 | Gly | Gly | NMePhe | Tyr | Ahp | Trp | D-Ala | Tyr | Asp | D-Pro |
| A65 | 65 | Gly | Gly | NMePhe | Tyr | Ahp | Trp | Gly | Tyr | Asp | D-NMeA |
| A66 | 66 | Gly | Gly | NMePhe | Tyr | Ahp | Trp | Gly | Tyr | Asp | Pi(2R-COOH) |
| A67 | 67 | Gly | D-Ser | NMePhe | Tyr | Ahp | Trp | Gly | Tyr | Asp | D-Pro |
| A68 | 68 | Gly | Gly | NMePhe | Tyr | Ahp | Trp | Gly | Tyr | Asp | NMeGly |
| A69 | 69 | D-Ser | Gly | NMePhe | Tyr | Ahp | Trp | Gly | Tyr | Asp | D-Pro |
| A70 | 70 | Gly | Gly | NMePhe | Tyr | Ahp | Trp | Gly | Tyr | Asp | D-aMeP |
| A71 | 71 | Gly | Gly | NMePhe | Tyr | Ahp | Trp | Gly | Tyr | Glu | D-Pro |
| A72 | 72 | Gly | Gly | NMePhe | Tyr | Ahp | Trp | Gly | Tyr | aMeD | D-Pro |
| A73 | 73 | NMeGly | Gly | NMePhe | Tyr | Ahp | Trp | Gly | Tyr | Asp | D-Pro |
| A74 | 74 | Gly | Gly | NMePhe | Tyr | Ahp | Trp | Gly | NBnG | Asp | D-Pro |
| A75 | 75 | Gly | Gly | NMePhe | F(4am) | Aoc | Trp | Gly | K(COpipzaa) | Asp | D-Pro |
| A76 | 76 | Gly | Gly | NMePhe | F(4CCH) | Aoc | Trp(Boc) | Gly | K(COpipzaa)(OtBu) | Asp(OMpe) | D-Pro |
| B1 | 77 | Gly | Gly | NMePhe | F(4amPEG2NHDOTA) | Aoc | Trp | Gly | K(COpipzaa) | Asp | D-Pro |
| B2 | 78 | Gly | Gly | NMePhe | F(4ampEG2NHDOTA) | hS(Pr) | Trp | Gly | K(COpipzaa) | Asp | D-Pro |
| B3 | 79 | Gly | Gly | NMePhe | F(4OEtNHDOTA) | Ahp | Trp | Gly | K(COpipzaa) | Asp | D-Pro |
| B4 | 80 | Gly | Gly | NMe3PyA | F(4amPEG2NHDOTA) | hS(Pr) | W7N | Gly | K(COpipzaa) | Asp | D-Pro |
| B5 | 81 | hSer | Gly | NMePhe | F(4amPEG2NHDOTA) | hS(Pr) | Trp | Gly | K(COpipzaa) | Asp | D-Pro |
| B6 | 82 | Glu | Gly | NMePhe | F(4amPEG2NHDOTA) | hS(Pr) | Trp | Gly | K(COpipzaa) | Asp | D-Pro |
| B7 | 83 | hSer | Gly | NMe3PyA | F(4amPEG2NHDOTA) | hS(Pr) | W7N | Gly | K(COpipzaa) | Asp | D-Pro |
| B8 | 84 | Glu | Gly | NMe3PyA | F(4amPEG2NHDOTA) | hS(Pr) | W7N | Gly | K(COpipzaa) | Asp | D-Pro |
| B9 | 85 | Gly | Gly | NMePhe | F4(amMeGlyDOTA) | Ahp | Trp | Gly | Tyr | Asp | D-Pro |
| B10 | 86 | Gly | Gly | NMePhe | F4(amGlyDOTA) | Ahp | Trp | Gly | Tyr | Asp | D-Pro |
| B11 | 87 | Gly | Gly | NMePhe | F4(amBAlaDOTA) | Ahp | Trp | Gly | Tyr | Asp | D-Pro |
| B12 | 88 | Gly | Gly | NMePhe | F4(amMeBAlaDOTA) | Ahp | Trp | Gly | Tyr | Asp | D-Pro |
| B13 | 89 | Pro | Gly | NMePhe | F(4amPEG2NHDOTA) | Aoc | Trp | Gly | K(COpipzaa) | Asp | D-Pro |
| B14 | 90 | Gly | Gly | NMePhe | F(4amPEG2NHDOTA) | Ahp | Trp | Gly | K(COpipzaa) | Asp | D-Pro |
| B15 | 91 | Gly | Gly | NMePhe | F4(MeTriazolylMeNHDOTA) | Ahp | Trp | Gly | Tyr | Asp | D-Pro |
| B16 | 92 | Gly | Gly | NMePhe | F(4amPEG2NHDOTA) | Ahp | Trp | Gly | Tyr | Asp | D-Pro |
| B17 | 93 | Gly | Gly | NMePhe | F(4amDOTA) | Ahp | Trp | Gly | Tyr | Asp | D-Pro |
| B18 | 94 | Gly | Gly | NMePhe | F(4amPEG2NHDOTA) | Aoc | Trp | Gly | F(4OMe) | Asp | D-Pro |
| B19 | 95 | Gly | Gly | NMePhe | F(4amPEG2NHDOTA | Aoc | Trp | Gly | F(3OH) | Asp | D-Pro |
| B20 | 96 | Gly | Gly | NMePhe | F(4amPEG2NHDOTA) | Q(Pyrro(2CN4F2)) | Trp | Gly | K(COpipzaa) | Asp | D-Pro |
| B21 | 97 | Pro | Gly | NMePhe | F(4amPEG2NHDOTA) | hS(Pr) | Trp | Gly | K(COpipzaa) | Asp | D-Pro |
| B22 | 98 | Lys(DOTA) | Gly | NMePhe | Tyr | Ahp | Trp | Gly | Tyr | Asp | D-Pro |
| B23 | 99 | Gly | Gly | NMePhe | F(4OEtNHDOTA) | Ahp | Trp | Gly | Tyr | Asp | D-Pro |
| B24 | 100 | Gly | Gly | NMePhe | F(4OEtNHDOTA) | Ahp | Trp | Gly | 3PyA(6NH2) | Asp | D-Pro |
| B25 | 101 | K(COpipzaa) | Gly | NMePhe | F(4OEtNHDOTA) | Ahp | Trp | Gly | Tyr | Asp | D-Pro |
| B26 | 102 | hSer | Gly | NMePhe | F(4OEtNHDOTA) | Ahp | Trp | Gly | Tyr | Asp | D-Pro |
| B27 | 103 | Gly | Gly | NMePhe | F(4amPEG2NHDOTA) | Aoc | W7N | Gly | K(COpipzaa) | Asp | D-Pro |
| B28 | 104 | Gly | Gly | NMe3PyA | F(4amPEG2NHDOTA) | Aoc | Trp | Gly | K(COpipzaa) | Asp | D-Pro |
| B29 | 105 | Gly | Gly | NMePhe | F(4OEtNHNODAGA) | Ahp | Trp | Gly | Tyr | Asp | D-pro |
| B30 | 106 | Gly | Gly | NMePhe | F(4OEtNHRDOTAGA) | Ahp | Trp | Gly | Tyr | Asp | D-Pro |
| B31 | 107 | Gly | Gly | NMePhe | F(4amPEG2NHAAZTA) | hS(Pr) | Trp | Gly | K(COpipzaa) | Asp | D-Pro |
| B32 | 108 | Gly | Gly | NMePhe | F(4amPEG2NHRDOTAGA) | hS(Pr) | Trp | Gly | K(COpipzaa) | Asp | D-Pro |
| B33 | 109 | Gly | Gly | NMePhe | F(4amPEG2NHNODAGA) | hS(pr) | Trp | Gly | K(COpipzaa) | Asp | D-Pro |
| B34 | 110 | Gly | Gly | NMePhe | F(4amPEG2NHNOTA | hS(Pr) | Trp | Gly | K(COpipzaa) | Asp | D-Pro |
| B35 | 111 | Gly | Gly | NMePhe | F(4amPEG2NH-p-SCN-DOTA | hS(Pr) | Trp | Gly | K(COpipzaa) | Asp | D-Pro |
| B36 | 112 | Gly | Gly | NMePhe | F(4amPEG2NHRDOTAGA) | Aoc | Trp | Gly | K(COpipzaa) | Asp | D-Pro |
| B37 | 113 | Gly | Gly | NMePhe | F(4amPEG2NH-p-SCN-DOTA | Aoc | Trp | Gly | K(COpipzaa) | Asp | D-Pro |
| B38 | 114 | Gly | Gly | NMePhe | F(4amPEG2NHNODAGA) | Aoc | Trp | Gly | K(COpipzaa) | Asp | D-Pro |
| B39 | 115 | Gly | Gly | NMePhe | F(4amPEG2NHAAZTA) | Ao | Trp | Gly | K(COpipzaa) | Asp | D-Pro |
| B40 | 116 | Gly | Gly | NMePhe | F(4amPEG2NHNOTA) | Aoc | Trp | Gly | K(COpipzaa) | Asp | D-Pro |
| C1 | 117 | Gly | Gly | NMePhe | F(4amPEG2NHDOTA-Lu) | Aoc | Trp | Gly | K(COpipzaa) | Asp | D-Pro |
| C2 | 118 | Gly | Gly | NMePhe | F(4amPEG2NHDOTA-Lu) | hS(Pr) | Trp | Gly | K(COpipzaa) | Asp | D-Pro |
| C3 | 119 | Gly | Gly | NMe3PyA | F(4amPEG2NHDOTA-Lu) | hS(Pr) | W7N | Gly | K(COpipzaa) | Asp | D-Pro |
| C4 | 120 | hSer | Gly | NMePhe | F(4amPEG2NHDOTA-Lu) | hS(Pr) | Trp | Gly | K(COpipzaa) | Asp | D-Pro |
| C5 | 121 | Glu | Gly | NMePhe | F(4amPEG2NHDOTA-Lu) | hS(Pr) | Trp | Gly | K(COpipzaa) | Asp | D-Pro |
| C6 | 122 | hSer | Gly | NMe3PyA | F(4amPEG2NHDOTA-Lu) | hS(Pr) | W7N | Gly | K(COpipzaa) | Asp | D-Pro |
| C7 | 123 | Glu | Gly | NMe3PyA | F(4amPEG2NHDOTA-Lu) | hS(Pr) | W7N | Gly | K(COpipzaa) | Asp | D-Pro |
| C8 | 124 | Gly | Gly | NMePhe | F4(amMeGlyDOTALu) | Ahp | Trp | Gly | Tyr | Asp | D-Pro |
| C9 | 125 | Gly | Gly | NMePhe | F4(amGlyDOTALu) | Ahp | Trp | Gly | Tyr | Asp | D-Pro |
| C10 | 126 | Gly | Gly | NMePhe | F4(amBAlaDOTALu) | Ahp | Trp | Gly | Tyr | Asp | D-Pro |
| C11 | 127 | Gly | Gly | NMePhe | F4(amMeBAlaDOTALu) | Ahp | Trp | Gly | Tyr | Asp | D-Pro |
| C12 | 128 | Pro | Gly | NMePhe | F(4amPEG2NHDOTA-Lu) | Aoc | Trp | Gly | K(COpipzaa) | Asp | D-Pro |
| C13 | 129 | Gly | Gly | NMePhe | F(4amPEG2NHDOTA-Lu) | Ahp | Trp | Gly | K(COpipzaa) | Asp | D-Pro |
| C14 | 130 | Gly | Gly | NMePhe | F4(METRIAZOLYLMENHDOTA-Lu) | Ahp | Trp | Gly | Tyr | Asp | D-Pro |
| C15 | 131 | Gly | Gly | NMePhe | F(4amPEG2NHDOTA-Lu) | Ahp | Trp | Gly | Tyr | Asp | D-Pro |
| C16 | 132 | Gly | Gly | NMePhe | F(4amDOTA-Lu) | Ahp | Trp | Gly | Tyr | Asp | D-Pro |
| C17 | 133 | Gly | Gly | NMePhe | F(4amPEG2NHDOTA-Lu) | Aoc | Trp | Gly | F(4OMe) | Asp | D-Pro |
| C18 | 134 | Gly | Gly | NMePhe | F(4amPEG2NHDOTA-Lu) | Aoc | Trp | Gly | F(3Oh) | Asp | D-Pro |
| C19 | 135 | Gly | Gly | NMePhe | F(4amPEG2NHDOTA-Lu) | Q(Pyrro(2CN4F2)) | Trp | Gly | K(COpipzaa) | Asp | D-Pro |
| C20 | 136 | Pro | Gly | NMePhe | F(4amPEG2NHDOTA-Lu) | hS(Pr) | Trp | Gly | K(COpipzaa) | Asp | D-Pro |
| C21 | 137 | Gly | Gly | NMePhe | F(4amPEG2NHDOTA-Lu) | hS(Pr) | W7N | Gly | K(COpipzaa) | Asp | D-Pro |
| C22 | 138 | Lys(DOTA-Lu) | Gly | NMePhe | Tyr | Ahp | Trp | Gly | Tyr | Asp | D-Pro |
| C23 | 139 | Gly | Gly | NMePhe | F(4OEtNHDOTA-Lu) | Ahp | Trp | Gly | Tyr | Asp | D-Pro |
| C24 | 140 | Gly | Gly | NMePhe | F(4OEtNHDOTA-Lu) | Ahp | Trp | Gly | 3PyA(6NH2) | Asp | D-Pro |
| C25 | 141 | K(COpipzaa) | Gly | NMePhe | F(4OEtNHDOTA-Lu) | Ahp | Trp | Gly | Tyr | Asp | D-Pro |
| C26 | 142 | hSer | Gly | NMePhe | f(4OEtNHDOTA-Lu) | Ahp | Trp | Gly | Tyr | Asp | D-Pro |
| C27 | 143 | Gly | Gly | NMePhe | F(4OEtNHDOTA-Lu) | Ahp | Trp | Gly | K(COpipzaa) | Asp | D-Pro |
| C28 | 144 | Gly | Gly | NMePhe | F(4amPEG2NHDOTA-Lu) | Aoc | W7N | Gly | K(COpipzaa) | Asp | D-Pro |
| C29 | 145 | Gly | Gly | NMe3PyA | F(4amPEG2NHDOTA-Lu) | Aoc | Trp | Gly | K(COpipzaa) | Asp | D-Pro |
| C30 | 146 | Gly | Gly | NMePhe | F(4OEtnHNODAGA-Lu) | Ahp | Trp | Gly | Tyr | Asp | D-Pro |
| C31 | 147 | Gly | Gly | NMePhe | F(4OEtNHRDOTAGA-Lu) | Ahp | Trp | Gly | Tyr | Asp | D-Pro |
| C32 | 148 | Gly | Gly | NMePhe | F(4amPEG2NHAAZTA-Lu) | hS(Pr) | Trp | Gly | K(COpipzaa) | Asp | D-Pro |
| C33 | 149 | Gly | Gly | NMePhe | F(4amPEG2NHRDOTAGA-Lu) | hS(Pr) | Trp | Gly | K(COpipzaa) | Asp | D-Pro |
| C34 | 150 | Gly | Gly | NMePhe | F(4amPEG2NHRDOTAGA-Lu) | Aoc | Trp | Gly | K(COpipzaa) | Asp | D-Pro |
| C35 | 151 | Gly | Gly | NMePhe | F(4amPEG2NH-p-ScN-DOTA-Lu) | Aoc | Trp | Gly | K(COpipzaa) | Asp | D-Pro |
| C36 | 152 | Gly | Gly | NMePhe | F(4amPEG2NHNODaGA-Lu) | Aoc | Trp | Gly | K(COpipzaa) | Asp | D-Pro |
| C37 | 153 | Gly | Gly | NMePhe | F(4amPEG2NHAAZTA-lu) | Aoc | Trp | Gly | K(COpipzaa) | Asp | D-Pro |
| C38 | 154 | Gly | Gly | NMePhe | F(4OEtNHRDOTAGA-Ga) | Ahp | Trp | Gly | Tyr | Asp | D-Pro |
| C39 | 155 | Gly | Gly | NMePhe | F(4amPEG2NHRDOTAGA-Ga) | hS(Pr) | Trp | Gly | K(COpipzaa) | AsI | D-Pro |
| C40 | 156 | Gly | Gly | NMePhe | F(4amPEG2NHNODAGA-Ga) | hS(Pr) | Trp | Gly | K(COpipzaa) | Asp | D-Pro |
| C41 | 157 | Gly | Gly | NMePhe | F(4amPEG2NH-NOTA-Ga) | hS(Pr) | Trp | Gly | K(COpipzaa) | Asp | D-Pro |
| C42 | 158 | Gly | Gly | NMePhe | F(4amPEG2NHNODAGA-Ga) | Aoc | Trp | Gly | K(COpipzaa) | Asp | D-Pro |
| C43 | 159 | Gly | Gly | NMePhe | F(4amPEG2NH-NOTA-Ga) | Aoc | Trp | Gly | K(COpipzaa) | Asp | D-Pro |
| C44 | 160 | Gly | Gly | NMePhe | F(4amPEG2NHDOTA-Ga) | Aoc | Trp | Gly | K(COpipzaa) | Asp | D-Pro |
| C45 | 161 | Glu | Gly | NMePhe | F(4amPEG2NHDOTA-Ga) | hS(Pr) | Trp | Gly | K(COpipzaa) | Asp | D-Pro |
| C46 | 162 | Gly | Gly | NMePhe | F(4amPeG2NHDOTA-Ga) | Ahp | Trp | Gly | K(COpipzaa) | asp | D-Pro |
| C47 | 163 | Pro | Gly | NMePhe | F(4amPEG2NHDOTA-Ga) | Aoc | Trp | Gly | K(COpipzaa) | Asp | D-Pro |
| C48 | 164 | Gly | Gly | NMe3PyA | F(4amPEG2NHDOTA-Ga) | Aoc | Trp | Gly | K(Copipzaa) | Asp | D-Pro |
| C49 | 165 | Gly | Gly | NMePhe | F(4amPEG2NHDOTA-Ga) | Ahp | Trp | Gly | Tyr | Asp | D-Pro |
| C50 | 166 | Gly | Gly | NMePhe | F(4OEtNHDOTA-Ga) | Ahp | Trp | Gly | K(COpipzaa) | Asp | D-Pro |
| C51 | 167 | Gly | Gly | NMePhe | F(4OEtNHDOTA-Ga) | Ahp | Trp | Gly | Tyr | Asp | D-Pro |
| C52 | 168 | Gly | Gly | NMePhe | F(4amPEG2NHDOTA-Ga) | hS(Pr) | Trp | Gly | K(COpipzaa) | Asp | D-Pro |
| C53 | 169 | Gly | Gly | NMePhe | F(4amPEG2NH-NOTA-AlF) | hS(Pr) | Trp | Gly | K(COpipzaa) | Asp | D-Pro |
| C54 | 170 | Gly | Gly | NMePhe | F(4amPEG2NH-NOTA-AlF) | Aoc | Trp | Gly | K(COpipzaa) | Asp | D-Pro |
| C55 | 171 | Gly | Gly | NMePhe | F(4amPEG2NHCOPy3(4F)) | Aoc | Trp | Gly | K(COpipzaa) | Asp | D-Pro |
| C56 | 172 | Gly | Gly | NMePhe | F(4amPEG2NHCOPy3(4F)) | hS(Pr) | Trp | Gly | K(Copipzaa) | Asp | D-Pro |
| C57 | 173 | Gly | Gly | NMePhe | F(4amPEG2NHCOPy3(4(NMe3))) | Aoc | Trp | Gly | K(COpipzaa) | Asp | D-Pro |
| C58 | 174 | Gly | Gly | NMePhe | F(4amPEG2NHCOPy3(4(NMe3))) | Aoc | Trp | Gly | K(COpipzaa) | Asp | D-Pro |
| D1 | 175 | Gly | Gly | NMePhe | F(4amPEG2NH-DOTA-Lu) | Aoc | Trp | Gly | Lys(Cy5) | Asp | D-Pro |
| D2 | 176 | Lys(Cy5) | Gly | NMePhe | F(4amPEG2NH-DOTA-Lu) | Aoc | Trp | Gly | K(COpipzaa) | Asp | D-Pro |
| F1 | 177 | Gly | Glu(Alb1) | NMePhe | F(4amPEG2NH-DOTA-Lu) | Aoc | Trp | Gly | K(COpipzaa) | Asp | D-Pro |
| F2 | 178 | Gly | Glu(Alb2) | NMePhe | F(4amPEG2NH-DOTA-Lu) | Aoc | Trp | Gly | K(COpipzaa) | Asp | D-Pro |
| G1 | 179 | Gly | Gly | NMePhe | F(4OEtNHDOTA-[177Lu]Lu) | Ahp | Trp | Gly | Tyr | Asp | D-Pro |
| G2 | 180 | Gly | Gly | NMePhe | F(4amPEG2NHDOTA-[177Lu]Lu) | Q(Pyrro(2CN4F2)) | Trp | Gly | K(COpipzaa) | Asp | D-Pro |
| G3 | 181 | Gly | Gly | NMePhe | F(4amPEG2NHDOTA[177Lu]Lu) | hS(Pr) | Trp | Gly | K(COpipzaa) | Asp | D-Pro |
| G4 | 182 | Gly | Gly | NMePhe | F(4amPEG2NHDOTA-[177Lu]Lu) | Aoc | Trp | Gly | K(COpipzaa) | Asp | D-Pro |
| H1 | 183 | Gly | Gly | NMePhe | F(4amPEG2NHDOTA-[68Ga]Ga) | hS(Pr) | Trp | Gly | K(COpipzaa) | Asp | D-Pro |
| H2 | 184 | Gly | Gly | NMePhe | F(4amPEG2NHDOTA[68Ga]Ga) | Aoc | Trp | Gly | K(COpipzaa) | Asp | D-Pro |
| I1 | 185 | Gly | Gly | NMePhe | F(4amPEG2NHCOPy3(4[¹⁸F]F)) | Aoc | Trp | Gly | K(COpipzaa) | Asp | D-Pro |
| J1 | 186 | Gly | Gly | NMePhe | F(4amPEG2NHDOTA-[²²⁵Ac]Ac) | Aoc | Trp | Gly | K(COpipzaa) | Asp | D-Pro |
| M1 | 187 | Gly | Gly | NMePhe | F((4amPEG2NH)2-bE-NHDOTA) | Aoc | Trp | Gly | K(COpipzaa) | Asp | D-Pro |
| M2 | 188 | Gly | Gly | NMePhe | F((4amPEG2NH)2-bE-NHDOTA-Lu) | Aoc | Trp | Gly | K(COpipzaa) | Asp | D-Pro |
| M3 | 189 | Gly | Gly | NMePhe | F((4amPEG2NH)2-bE-NHDOTA-Lu) | hS(Pr) | Trp | Gly | K(COpipzaa) | Asp | D-Pro |
| M4 | 190 | K(COpipzaa) | Gly | NMePhe | F((4OEtNHPEG3NH)2-bE-NHDOTA) | Ahp | Trp | Gly | Tyr | Asp | D-Pro |
| M5 | 191 | K(COpipzaa) | Gly | NMePhe | F((4amPEG2NH)2-bE-NHDOTA-Lu) | Ahp | Trp | Gly | Tyr | Asp | D-Pro |

The N-terminus is an NH₂ group and the C-terminus is a C(O)OH group for all peptides in Example III.

In certain embodiments, each of the peptides in Example III is a cyclic peptide, wherein A¹ and A¹⁰ are joined (attached) to form a cyclic peptide.

Those skilled in the art will recognize, or be able to ascertain, using no more than routine experimentation, numerous equivalents to the specific embodiments described specifically herein. Such equivalents are intended to be encompassed in the scope of the following claims.

1. A compound, or a pharmaceutically acceptable salt, solvate, stereoisomer, or tautomer thereof, comprising: 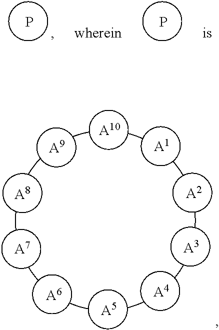
a) at least one cyclized peptide
wherein
A¹ is 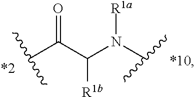
wherein
R^{1a} is selected from the group consisting of H and C₁₋₃-alkyl;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH2, —NHC₁₋₆-alkyl, —N(C₁₋₆-alkyl)₂ and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1b} is optionally substituted with 1, 2, 3, or 4 substituents independently selected from the group consisting of C₁₋₃-alkyl, —NH₂, —NHC₁₋₆-alkyl, —N(C₁₋₆-alkyl)₂, —OH, halo, and C(O)R^{1d};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH;
R^{1d} is selected from the group consisting of H, —OH, C₁₋₆-alkyl, —NHC₁₋₆-alkyl, and —OC₁₋₆-alkyl; and
*2 indicates the point of attachment to A² and *10 indicates the point of attachment to A¹⁰;
A² is 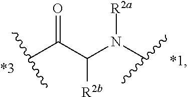
wherein
R^{2a} is selected from the group consisting of H and C₁₋₃-alkyl;
R^{2b} is selected from the group consisting of H and C₁₋₆-alkyl, wherein the C₁₋₆-alkyl of R^{2b} is optionally substituted with 1, 2, 3, or 4 substituents independently selected from the group consisting of —C(O)OH, —OH, —NH₂, —NHC₁₋₆-alkyl, and —N(C₁₋₆-alkyl)₂; and
*1 indicates the point of attachment to A¹ and *3 indicates the point of attachment to A³;
A³ is 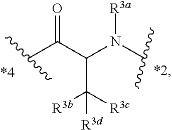
wherein
R^{3a} is selected from the group consisting of H and C₁₋₃-alkyl;
R^{3b} is selected from the group consisting of H, C₁₋₃-alkyl, and halo;
R^{3c} is selected from the group consisting of halo, C₁₋₆ alkyl, C₃₋₈-cycloalkyl, phenyl, and 5-6 membered heteroaryl, wherein the C₁₋₆ alkyl, phenyl, and 5-6 membered heteroaryl of R³ are optionally substituted with 1, 2, 3, or 4 substituents independently selected from the group consisting of halo, —C(O)OH, —C(O)C₁₋₆-alkyl, —OH, —NHC₁₋₆-alkyl, —N(C₁₋₆-alkyl)₂, and —NH₂;
R^{3d} is selected from the group consisting of H, C₁₋₃-alkyl, and halo; and
*2 indicates the point of attachment to A² and *4 indicates the point of attachment to A⁴;
A⁴ is 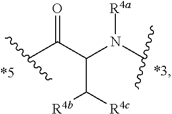
wherein
R^{4a} is selected from the group consisting of H and C₁₋₃-alkyl;
R^{4b} is selected from the group consisting of H and C₁₋₃-alkyl;
R^{4c} is selected from the group consisting of phenyl and 5-6 membered heteroaryl wherein phenyl and 5-6 membered heteroaryl of R^{4c} are optionally substituted with 1, 2, 3, 4, or 5 substituents R^{4d};
each R^{4d} is independently selected from C₁₋₆-alkyl, —OH, —OC₁-C₆-alkyl, —NH₂, halo, —NHC(O)R^{4f}, —C(O)NHR^{4f}, and —C(O)OR^{4f}, wherein the C₁₋₆-alkyl and —OC₁-C₆-alkyl of R^{4d} are each optionally substituted with 1, 2, or 3 substituents R^{4c};
each R^{4c} is independently selected from —CN, —NH₂, —NHC₁₋₆-alkyl, —N(C₁₋₆-alkyl)₂, —NHC(O)—(C₁₋₆-alkyl), —N₃, —OH, —C(O)OH, —C(O)NH₂, and 5-6 membered heteroaryl, wherein the 5-6 membered heteroaryl of R^{4c} is optionally substituted with —C₁₋₆-alkyl-NH₂ or —C₁₋₃-alkyl-OH;
each R^{4f} is independently selected from the group consisting of H and C₁₋₆-alkyl; and
*3 indicates the point of attachment to A³ and *5 indicates the point of attachment to A⁵;
A⁵ is 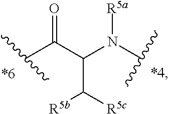
wherein
R^{5a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, and —C(O)R_{5d};
R^{5d} is 4-7 membered heterocycloalkyl optionally substituted with 1, 2, 3, or 4 groups independently selected from C₁₋₃-alkyl, —CN, halo, and —OH; and
wherein *4 indicates the point of attachment to A⁴ and *6 indicates the point of attachment to A⁶;
A⁶ is 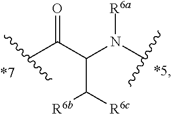
wherein
R^{6a} is selected from the group consisting of H and C₁₋₃-alkyl;
R^{6b} is selected from the group consisting of H and C₁₋₃-alkyl;
R^{6c} is a 5-10 membered heteroaryl optionally substituted with 1, 2, 3, or 4 groups independently selected from C₁₋₃-alkyl, —CN, halo, —NH₂, —NHC₁₋₆-alkyl, —N(C₁₋₆-alkyl)₂, —OH, and —OC₁₋₆-alkyl; and
wherein *5 indicates the point of attachment to A⁵ and *7 indicates the point of attachment to A⁷;
A⁷ is 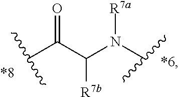
wherein
R^{7a} is selected from the group consisting of H and C₁₋₃-alkyl;
R^{7b} is selected from the group consisting of H and C₁₋₆-alkyl; and
wherein *6 indicates the point of attachment to A⁶ and *8 indicates the point of attachment to A⁸;
A⁸ is 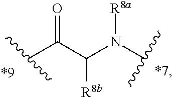
wherein
R^{8a} is selected from the group consisting of H, C₁₋₃-alkyl, and —CH₂-phenyl, wherein the C₁₋₃-alkyl and —CH₂-phenyl of R^{8a} are each optionally substituted with 1, 2, or 3 substituents independently selected from —NH₂, —NHC₁₋₆-alkyl, —N(C₁₋₆-alkyl)₂, —OH, —OC₁₋₃-alkyl, and C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R⁸′, —C₁₋₆-alkyl-O—C(O)R⁸′, and —C₁₋₆-alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH and
wherein *7 indicates the point of attachment to A⁷ and *9 indicates the point of attachment to A⁹;
A⁹ is 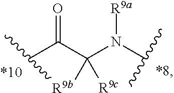
wherein
R^{9a} is selected from the group consisting of H and C₁₋₃-alkyl;
R^{9b} is selected from the group consisting of H and C₁₋₃-alkyl;
R^{9c} is C₁₋₆-alkyl optionally substituted with 1, 2, 3, or 4 groups independently selected from —CN, —C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
wherein *8 indicates the point of attachment to A⁸ and *10 indicates the point of attachment to A¹⁰;
A¹⁰ is 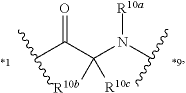
wherein
R^{10a} is selected from the group consisting of H and C₁₋₃-alkyl;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1, 2, 3, or 4 substituents independently selected from C₁₋₃-alkyl, —NH₂, —NHC₁₋₆-alkyl, —N(C₁₋₆-alkyl)₂, —OH, halo, and C(O)R^{10d};
R^{10d} is selected from the group consisting of H, —OH, C₁₋₆-alkyl and —OC₁₋₆-alkyl; and
wherein *1 indicates the point of attachment to A¹ and *9 indicates the point of attachment to A⁹;
and
b) at least one imaging agent, chelating agent, radionuclide, or cytotoxic drug, wherein the cyclized peptide 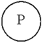
is conjugated to the at least one imaging agent, chelating agent, radionuclide, or cytotoxic drug via any one of A¹-A¹⁰, optionally through a linker.

2. The compound of claim 1, which is a compound of formula (I), (Ia), (Ib), or (Ic): 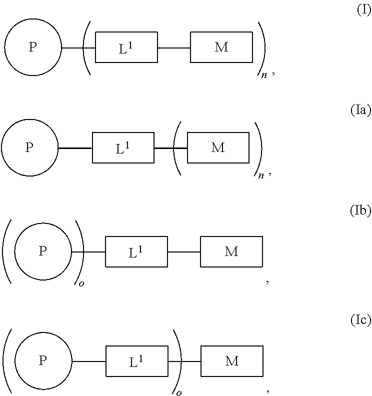
or a pharmaceutically acceptable salt, solvate, stereoisomer, or tautomer thereof, wherein
L¹ is, independently at each occurrence, a bond or a linker;
M is, independently at each occurrence, an imaging agent, a chelating agent, or a radionuclide,
wherein the chelating agent is optionally radiolabeled with a radionuclide;
n is 1, 2, 3, or 4; and
is 1, 2, 3, or 4,
wherein any of P, L¹, or M is optionally substituted with an albumin binder.

3. (canceled)

4. The compound of claim 2, or a pharmaceutically acceptable salt, solvate, stereoisomer, or tautomer thereof, wherein the compound of formula (I) is a compound of formula (I-i): 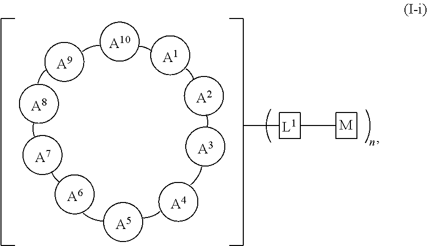
or a pharmaceutically acceptable salt, solvate, stereoisomer, or tautomer thereof,
wherein
L¹ is, independently at each occurrence, selected from the group consisting of a bond and a linker;
M is, independently at each occurrence, selected from the group consisting of an imaging agent, a chelating agent, and a radionuclide the chelating agent is optionally radiolabeled with a radionuclide;
n is 1, 2, 3, or 4; and
wherein any of A¹-A¹⁰ and L¹ is optionally substituted with an albumin binder.

5. The compound of claim 1, or a pharmaceutically acceptable salt, solvate, stereoisomer, or tautomer thereof, wherein:
A¹ is a moiety of formula (II): 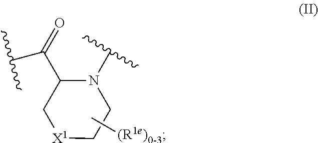
wherein:
X¹ is absent or selected from the group consisting of O, NH, CH₂, and CHR^{1e}; and each R^{1e} is independently selected from the group consisting of C₁₋₃-alkyl, —NH₂, —NHC₁₋₆-alkyl, —N(C₁₋₆-alkyl)₂, —OH, halo, and C(O)R^{1d}, wherein R^{1d} is selected from the group consisting of H and —C₁₋₆-alkyl; or
A¹⁰ is a moiety of formula (IIA): 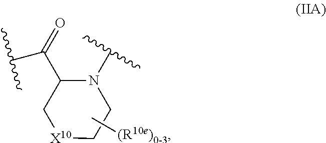
wherein:
X¹⁰ is absent or selected from the group consisting of O, NH, CH₂, and CHR^{10e}; and
each R^{10e} is independently selected from the group consisting of C₁₋₃-alkyl, —NH₂, —NHC₁₋₆-alkyl, —N(C₁₋₆-alkyl)₂, —OH, halo, and C(O)R^{10d}, wherein R^{10d} is selected from the group consisting of H, —OH, C₁₋₆-alkyl and —OC₁₋₆-alkyl;
provided that when A¹ is a moiety of formula (II), A¹⁰ is not a moiety of formula (IIA) and when A¹⁰ is a moiety of formula (IIA), A¹ is not a moiety of formula (II).

6. The compound of claim 1, or a pharmaceutically acceptable salt, solvate, stereoisomer, or tautomer thereof, wherein:
A⁴ is a moiety of formula (III): 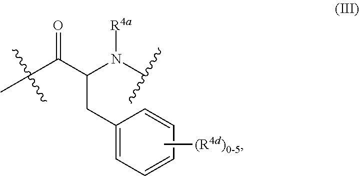
wherein:
R^{4a} is selected from the group consisting of H and C₁₋₃-alkyl,
each R^{4d} is independently selected from the group consisting of —C₁₋₆-alkyl, —OH, —OC₁₋₆-alkyl, —NH₂, halo, —NHC(O)R^{4f}, —C(O)NHR⁴, and —C(O)OR^{4f}, wherein the C₁₋₆-alkyl and —OC₁₋₆-alkyl of R^{4d} are each optionally substituted with 1, 2, or 3 substituents independently selected from the group consisting of —CN, —NH₂, —NHC₁₋₆-alkyl, —N(C₁₋₆-alkyl)₂, N₃, —OH, —C(O)OH, —C(O)NH₂, and 5-6 membered heteroaryl optionally substituted with —C₁₋₃-alkyl-NH₂ or —C₁₋₃-alkyl-OH; and
each R^{4f} is independently selected from the group consisting of H and C₁₋₆-alkyl.

7. The compound of claim 1, or a pharmaceutically acceptable salt, solvate, stereoisomer, or tautomer thereof, wherein:
A⁸ is a moiety of formula (IV): 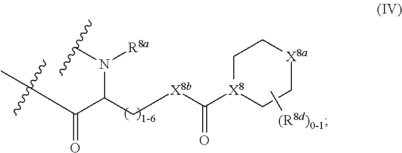
wherein:
X⁸ is selected from the group consisting of N and CH;
X^{8a} is selected from the group consisting of NH, NR^{8d}, O, CH₂, and CHR^{8d};
X^{8b} is selected from the group consisting of NH, NR^{8a}, O, and CH₂;
R^{8a} is selected from the group consisting of H and C₁₋₃-alkyl; and
R^{8d} is selected from the group consisting of —C₁₋₆-alkylC(O)OH and —C(O)OH;
wherein at least one of X⁸ and X^{8a} is N.

8. The compound of claim 1, or a pharmaceutically acceptable salt, solvate, stereoisomer, or tautomer thereof, wherein A¹ is selected from the group consisting of: 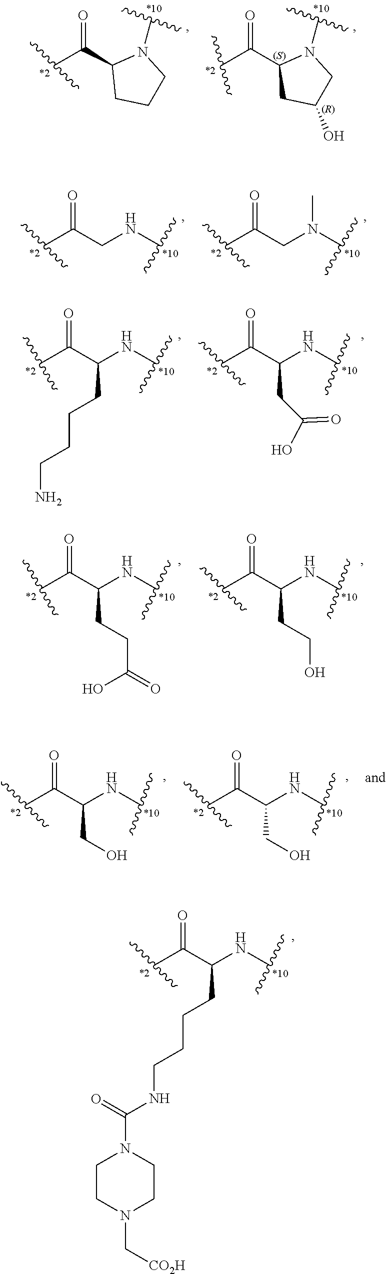
wherein *2 indicates the point of attachment to A² and *10 indicates the point of attachment to A¹⁰.

9. The compound of claim 1, or a pharmaceutically acceptable salt, solvate, stereoisomer, or tautomer thereof, wherein A² is selected from the group consisting of: 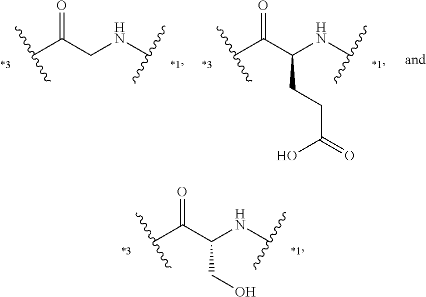 [SMILES: *NCC(*)=O ; *N[C@@H](CCC(=O)O)C(*)=O ; *N[C@H](CO)C(*)=O]
wherein *1 indicates the point of attachment to A¹ and *3 indicates the point of attachment to A³.

10. The compound of claim 1, or a pharmaceutically acceptable salt, solvate, stereoisomer, or tautomer thereof, wherein A³ is selected from the group consisting of: 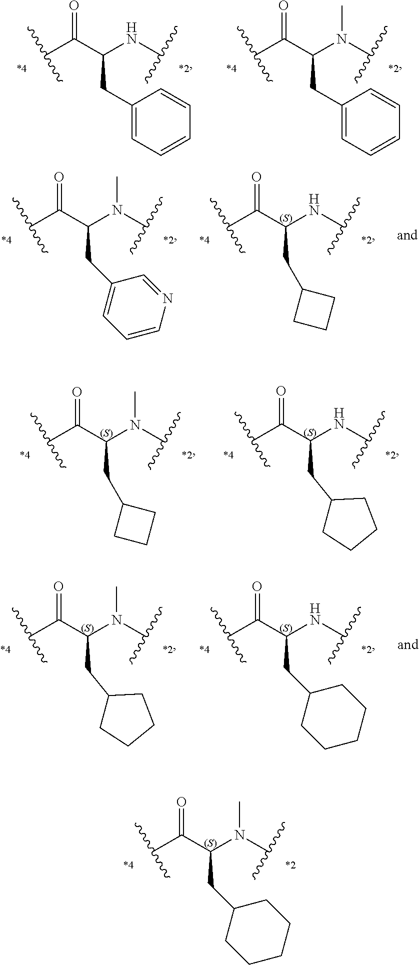
wherein *2 indicates the point of attachment to A² and *4 indicates the point of attachment to A⁴.

11. The compound of claim 1, or a pharmaceutically acceptable salt, solvate, stereoisomer, or tautomer thereof, wherein A⁴ is selected from the group consisting of: 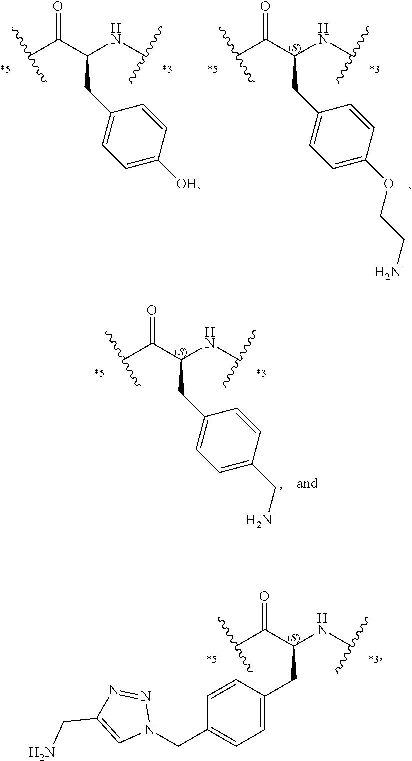
wherein *3 indicates the point of attachment to A³ and *5 indicates the point of attachment to A⁵.

12. The compound of claim 1, or a pharmaceutically acceptable salt, solvate, stereoisomer, or tautomer thereof, wherein A⁵ is selected from the group consisting of: 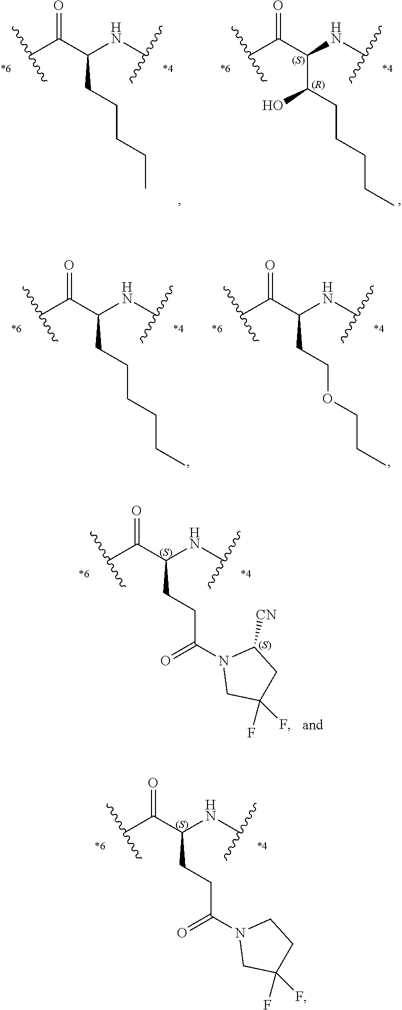
wherein *4 indicates the point of attachment to A⁴ and *6 indicates the point of attachment to A⁶.

13. The compound of claim 1, or a pharmaceutically acceptable salt, solvate, stereoisomer, or tautomer thereof, wherein A⁶ is selected from the group consisting of: 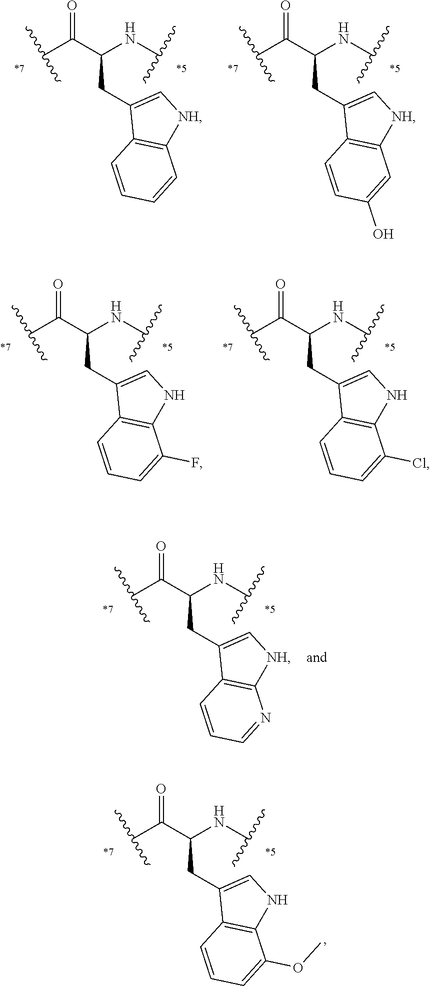
wherein *5 indicates the point of attachment to A⁵ and *7 indicates the point of attachment to A⁷.

14. The compound of claim 1, or a pharmaceutically acceptable salt, solvate, stereoisomer, or tautomer thereof, wherein A⁷ is selected from the group consisting of: 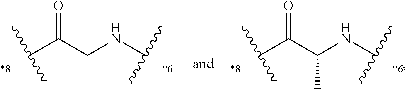
wherein *6 indicates the point of attachment to A⁶ and *8 indicates the point of attachment to A⁸.

15. The compound of claim 1, or a pharmaceutically acceptable salt, solvate, stereoisomer, or tautomer thereof, wherein A⁸ is selected from the group consisting of: 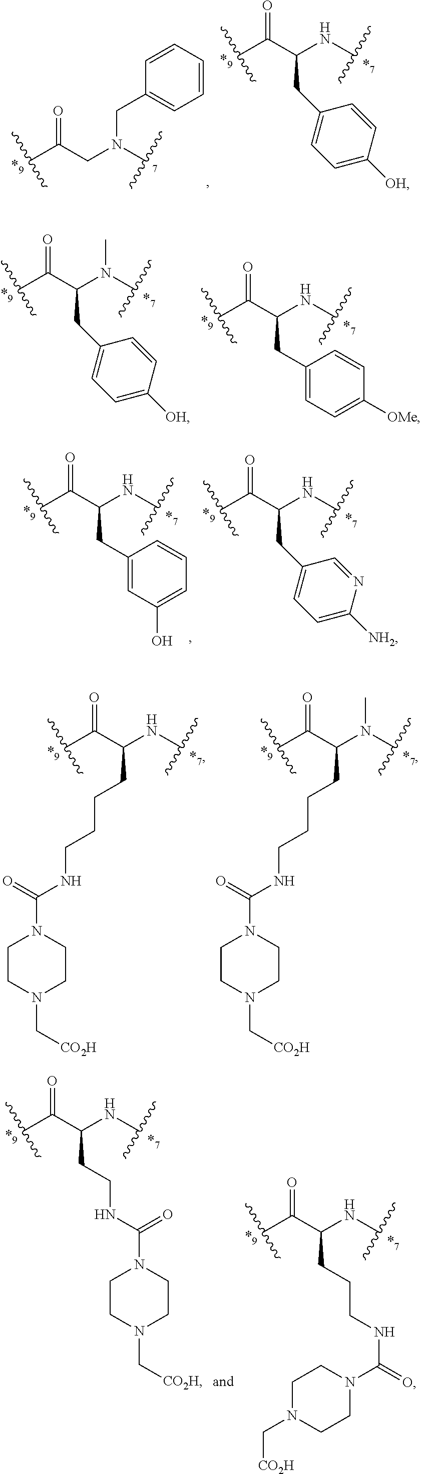
wherein *7 indicates the point of attachment to A⁷ and *9 indicates the point of attachment to A⁹.

16. The compound of claim 1, or a pharmaceutically acceptable salt, solvate, stereoisomer, or tautomer thereof, wherein A⁹ is selected from the group consisting of: 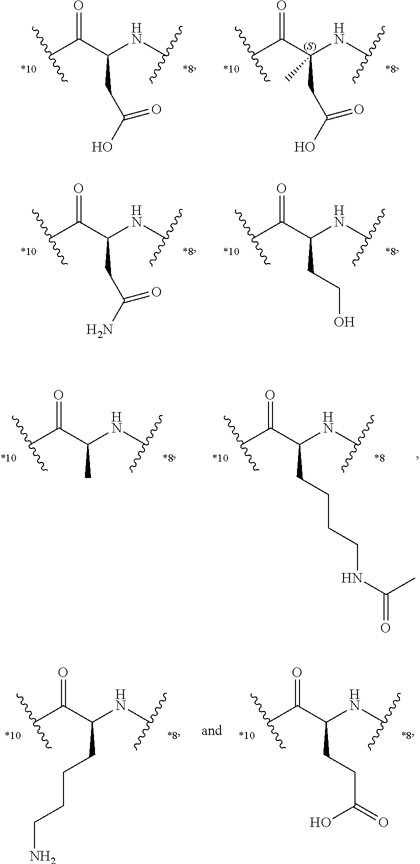
wherein *8 indicates the point of attachment to A⁸ and *10 indicates the point of attachment to A¹⁰.

17. The compound of claim 1, or a pharmaceutically acceptable salt, solvate, stereoisomer, or tautomer thereof, wherein A¹⁰ is selected from the group consisting of: 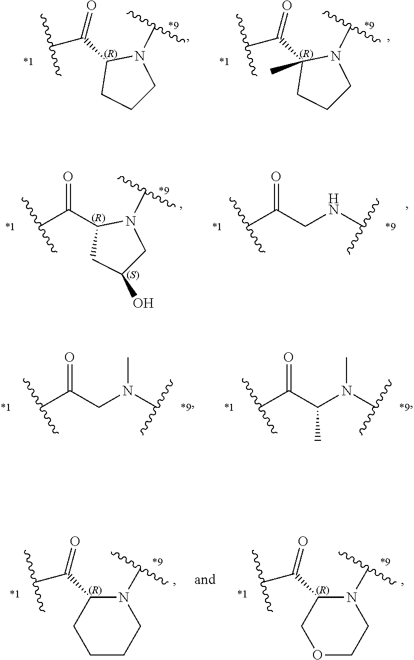
wherein *1 indicates the point of attachment to A¹ and *9 indicates the point of attachment to A⁹.

18. The compound of claim 1, or a pharmaceutically acceptable salt, solvate, stereoisomer, or tautomer thereof, wherein
A¹ is selected from the group consisting of 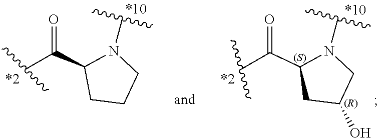
or
A¹⁰ is selected from the group consisting of

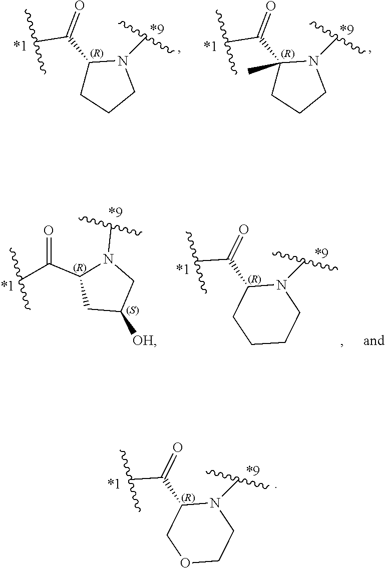

19-20. (canceled)

21. The compound of claim 1, or a pharmaceutically acceptable salt, solvate, stereoisomer, or tautomer thereof, wherein
(1) A¹ is a moiety of formula (II): 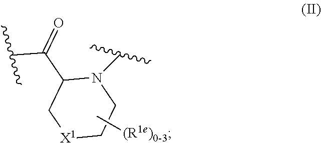
wherein:
X¹ is absent or selected from the group consisting of O, NH, CH₂, and CHR^{1e}; and
each R^{1e} is independently selected from the group consisting of C₁₋₃-alkyl, —NH₂, —NHC₁₋₆-alkyl, —N(C₁₋₆-alkyl)₂, —OH, halo, and C(O)R^{1d}, wherein R^{1d} is H or —C₁₋₆-alkyl; or
A¹⁰ is a moiety of formula (IIA): 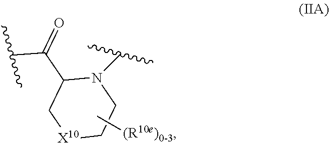
wherein:
X¹⁰ is absent or selected from the group consisting of O, NH, and CHR^{10e}; and
each R^{10e} is independently selected from the group consisting of C₁₋₃-alkyl, —NH₂, —NHC₁₋₆-alkyl, —N(C₁₋₆-alkyl)₂, —OH, halo, and C(O)R^{10d}, wherein R^{10d} is selected from the group consisting of H, —OH, C₁₋₆-alkyl and —OC₁₋₆-alkyl;
provided that when A¹ is a moiety of formula (II), A¹⁰ is not a moiety of formula (IIA) and when A¹⁰ is a moiety of formula (IIA), A¹ is not a moiety of formula (II);
(2) A⁴ is a moiety of formula (III): 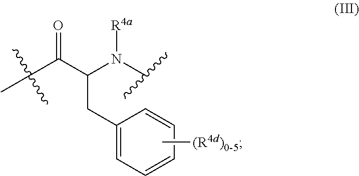
wherein:
R^{4a} is selected from the group consisting of H and C₁₋₃-alkyl,
each R^{4d} is independently selected from the group consisting of —C₁₋₆-alkyl, —OH, —OC₁₋₆-alkyl, —NH₂, halo, —NHC(O)R^{4f}, —C(O)NHR^{4f}, and —C(O)OR^{4f}, wherein the C₁₋₆-alkyl and —OC₁₋₆-alkyl of R^{4d} are each optionally substituted with 1, 2, or 3 substituents independently selected from the group consisting of —CN, —NH₂, —NHC₁₋₆-alkyl, —N(C₁₋₆-alkyl)₂, N₃, —OH, —C(O)OH, —C(O)NH₂, and 5-6 membered heteroaryl optionally substituted with —C₁₋₃-alkyl-NH₂ or —C₁₋₃-alkyl-OH; and
each R^{4f} is independently selected from the group consisting of H and C₁₋₆-alkyl; and
(3) A⁸ is a moiety of formula (IV): 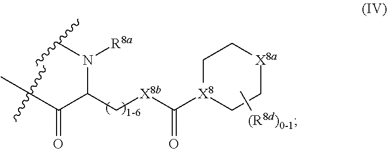
wherein:
X⁸ is selected from the group consisting of N and CH;
X^{8a} is selected from the group consisting of NH, NR^{8d}, O, CH₂, and CHR^{8d};
X^{8b} is selected from the group consisting of NH, NR^{8a}, O, and CH₂;
R^{8a} is selected from the group consisting of H and C₁₋₃-alkyl; and
R^{8d} is selected from the group consisting of —C₁₋₆-alkylC(O)OH and —C(O)OH;
wherein at least one of X⁸ and X^{8a} is N.

22-25. (canceled)

26. The compound of claim 1, or a pharmaceutically acceptable salt, solvate, stereoisomer, or tautomer thereof, wherein
A⁴ is: 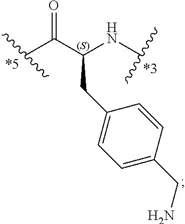
A⁸ is: 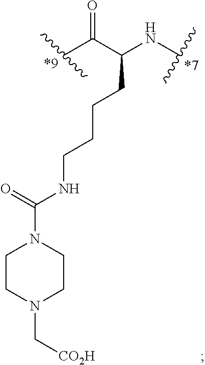 [SMILES: *N[C@@H](CCCCNC(=O)N1CCN(CC(=O)O)CC1)C(*)=O]
and
A¹⁰ is:

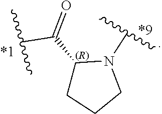

27. The compound of claim 2, wherein n is 1 or 2.

28. (canceled)

29. The compound of claim 2, or a pharmaceutically acceptable salt, solvate, stereoisomer, or tautomer thereof, wherein L¹ is a linker connected to any —NH₂ group of the cyclized peptide.

30. The compound of claim 2, wherein L¹ is a linker connected to a side chain of A¹, A², A⁴, or A⁸.

31. (canceled)

32. The compound of claim 2, or a pharmaceutically acceptable salt, solvate, stereoisomer, or tautomer thereof, wherein L¹ is a linker comprising alkylene, cycloalkylene, arylene, alkylarylene, heteroarylene, heterocycloalkylene, (CR⁴R⁵)ₚO(CR⁴R⁵)_{q}, (CR⁴R⁵)ₚN(CR⁴R⁵)_{q}, (CR⁴R⁵)ₚS(CR⁴R⁵)_{q}, amino acid, amino acid derivatives, or any combination thereof, wherein cycloalkylene, alkylene, arylene, alkylarylene, heteroarylene, and heterocycloalkylene of Lⁱ are each optionally substituted with 1, 2, or 3 substituents independently selected from halo, C₁₋₆ alkyl, C₂₋₆ alkenyl, C₂₋₆ alkynyl, C₁₋₆ haloalkyl, halosulfanyl, CN, NO₂, N₃, OR^{a}, SR^{a}, C(O)R, C(O)NR^{c}R^{d}, C(O)OR^{a}, OC(O)R^{b}, OC(O)NR^{c}R^{d}, NR^{c}R^{d} NR^{c}(O)R^{b}, NR^{c}C(O)NR^{c}R^{d} NR^{c}C(O)OR^{a}, C(═NR^{g})NR^{c}R^{d}, NR^{c}C(═NR^{g})NR^{c}R^{d}, P(R^{f})₂, P(OR^{e})₂, P(O)R^{e}R^{f}, P(O)OR^{e}OR^{f}, S(O)R^{b}, S(O)NR^{c}R^{d}, S(O)₂R^{b}, NR^{c}S(O)₂R^{b}, and S(O)₂NR^{c}R^{d},
wherein R⁴ and R⁵ are each independently selected from the group consisting of H, halo, OH, C₁₋₆ alkyl, C₂₋₆ alkenyl, C₂₋₆ alkynyl, C₁₋₆ alkoxy, alkoxyalkyl, cyanoalkyl, heterocycloalkyl, cycloalkyl, C₁₋₆ haloalkyl, CN, and NO₂;
R^{a}, R^{b}, R^{c}, and R^{d} are each independently selected from the group consisting of H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl of R^{a}, R^{b}, R^{c}, and R^{d} are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
R^{e}, R^{f}, and R^{g} are each independently selected from the group consisting of H and C₁₋₁₀ alkyl;
p is 0, 1, 2, 3, 4, 5, or 6; and
q is 0, 1, 2, 3, 4, 5, or 6.

33. The compound of claim 2, or a pharmaceutically acceptable salt, solvate, stereoisomer, or tautomer thereof, wherein L¹ is a linker comprising the following structure: 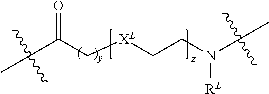
wherein:
X^{L} is selected from the group consisting of CH₂, N, and O;
R^{L} is selected from the group consisting of H, C₁₋₃-alkyl, and acyl;
y is 0, 1, 2, 3, 4, 5, or 6; and
z is 0, 1, 2, 3, or 4.

34. The compound of claim 2, or a pharmaceutically acceptable salt, solvate, stereoisomer, or tautomer thereof, wherein L¹ is a linker comprising one of the following structures: 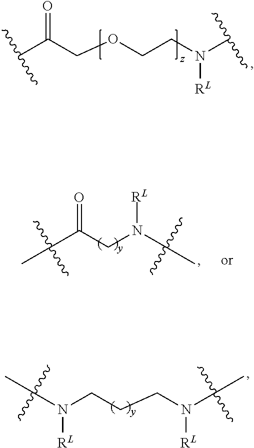
wherein:
R^{L} is selected from the group consisting of H and C₁₋₃-alkyl;
y is 0, 1, 2, 3, 4, 5, or 6; and
z is 0, 1, 2, 3, or 4.

35-39. (canceled)

40. The compound of claim 2, or a pharmaceutically acceptable salt, solvate, stereoisomer, or tautomer thereof, wherein L¹ is a linker comprising a structure selected from the group consisting of:

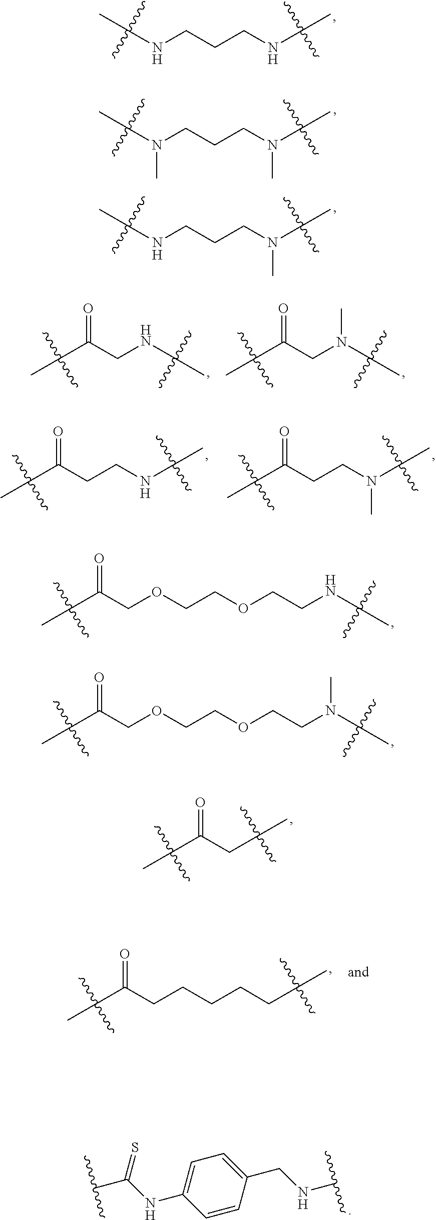

41. The compound of claim 2, or a pharmaceutically acceptable salt, solvate, stereoisomer, or tautomer thereof, wherein L¹ is a linker connected to a side chain of A⁴ and wherein A⁴ and L¹ together have a structure selected from:

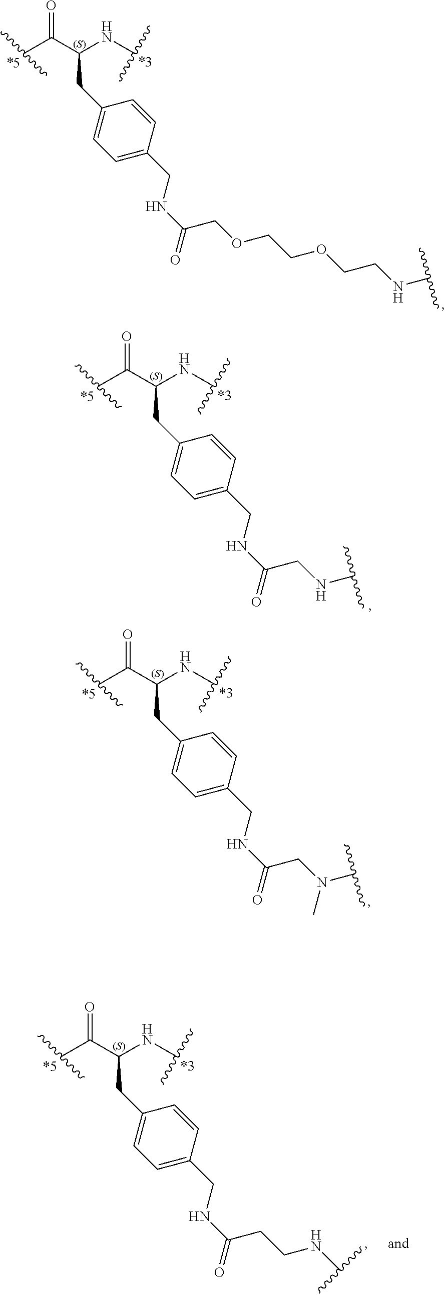

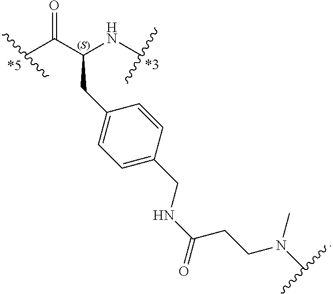

42-45. (canceled)

46. The compound of claim 2, or a pharmaceutically acceptable salt, solvate, stereoisomer, or tautomer thereof, wherein M is a chelating agent, wherein the chelating agent is optionally radiolabeled with a radionuclide.

47-50. (canceled)

51. The compound of claim 2, or a pharmaceutically acceptable salt, solvate, stereoisomer, or tautomer thereof, wherein M is a cyclic chelating agent selected from the group consisting of: 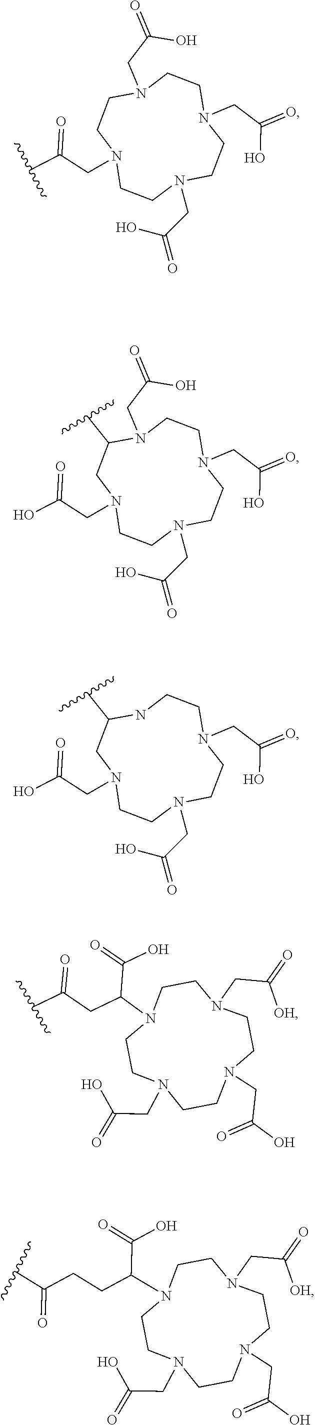 [SMILES: *C(=O)CN1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C1CN(CC(=O)O)CCN(CC(=O)O)CCN(CC(=O)O)CCN1 ; *C(=O)CC(C(=O)O)N1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1] 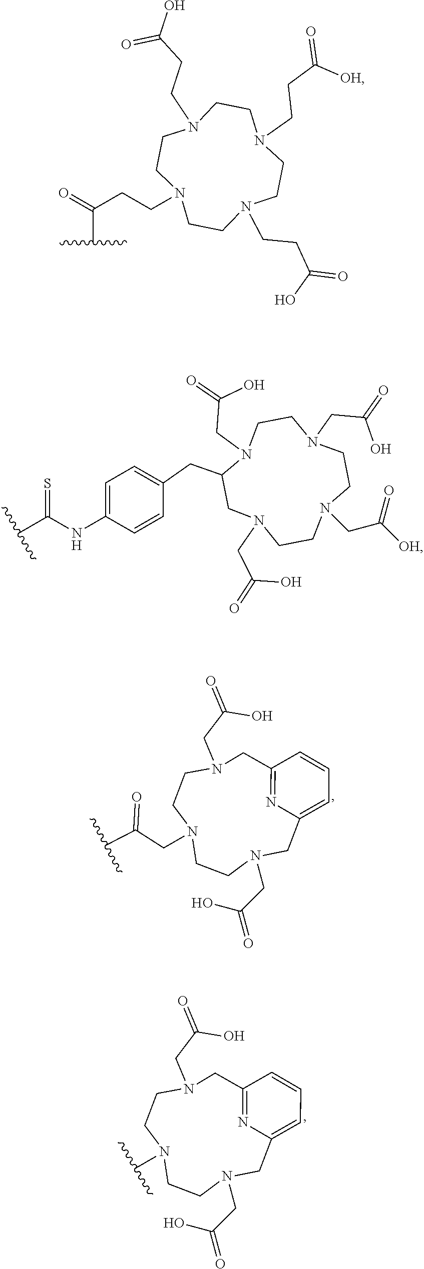 [SMILES: *C(=O)CCN1CCN(CCC(=O)O)CCN(CCC(=O)O)CCN(CCC(=O)O)CC1 ; *C(=S)Nc1ccc(CC2CN(CC(=O)O)CCN(CC(=O)O)CCN(CC(=O)O)CCN2CC(=O)O)cc1 ; *C(=O)CN1CCN(CC(=O)O)Cc2cccc(n2)CN(CC(=O)O)CC1 ; *N1CCN(CC(=O)O)Cc2cccc(n2)CN(CC(=O)O)CC1] 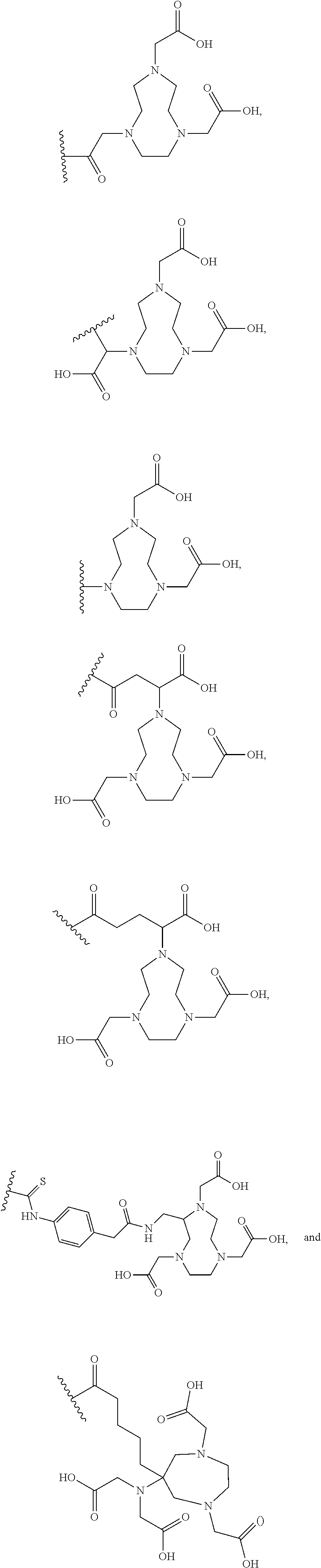
wherein the cyclic chelating agent is optionally radiolabeled with a radionuclide.

52. (canceled)

53. The compound of claim 2, or a pharmaceutically acceptable salt, solvate, stereoisomer, or tautomer thereof, wherein L¹ is a linker connected to a side chain of A⁴ and M is a cyclic chelating agent, wherein A⁴, L¹, and M together have a structure selected from the group consisting of:

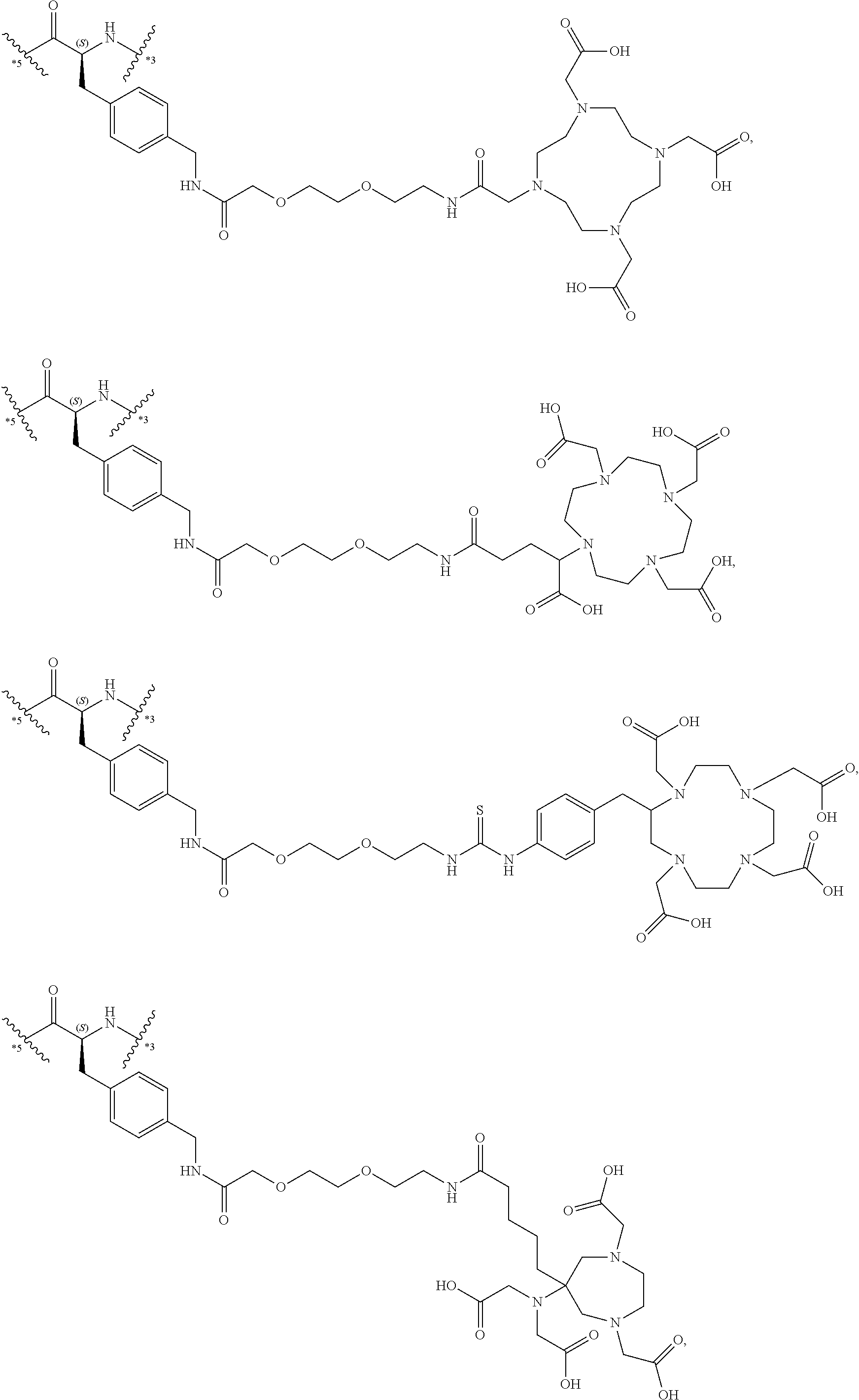

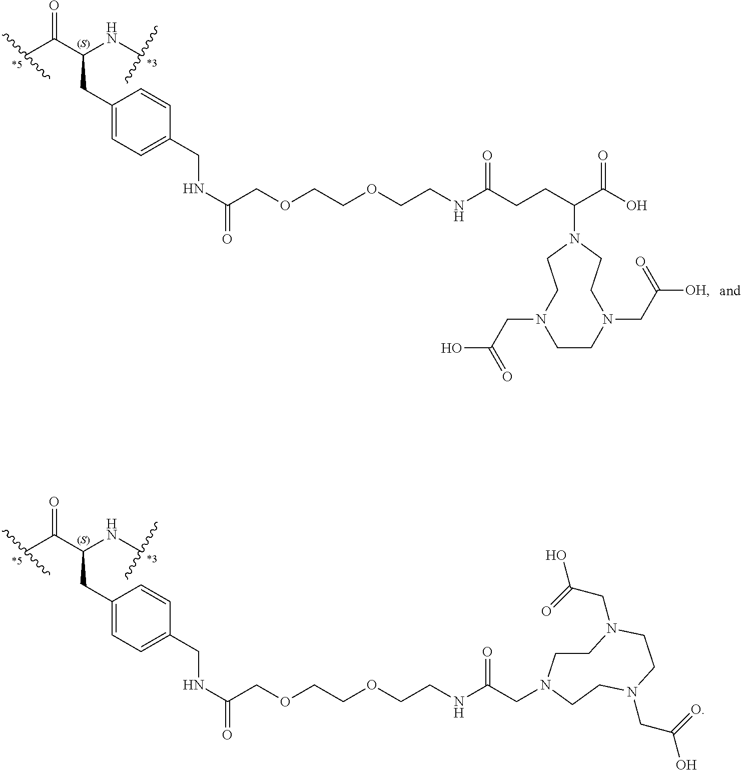

54-56. (canceled)

57. The compound of claim 1, or a pharmaceutically acceptable salt, solvate, stereoisomer, or tautomer thereof, comprising a radionuclide selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac.

58-63. (canceled)

64. A compound which is: 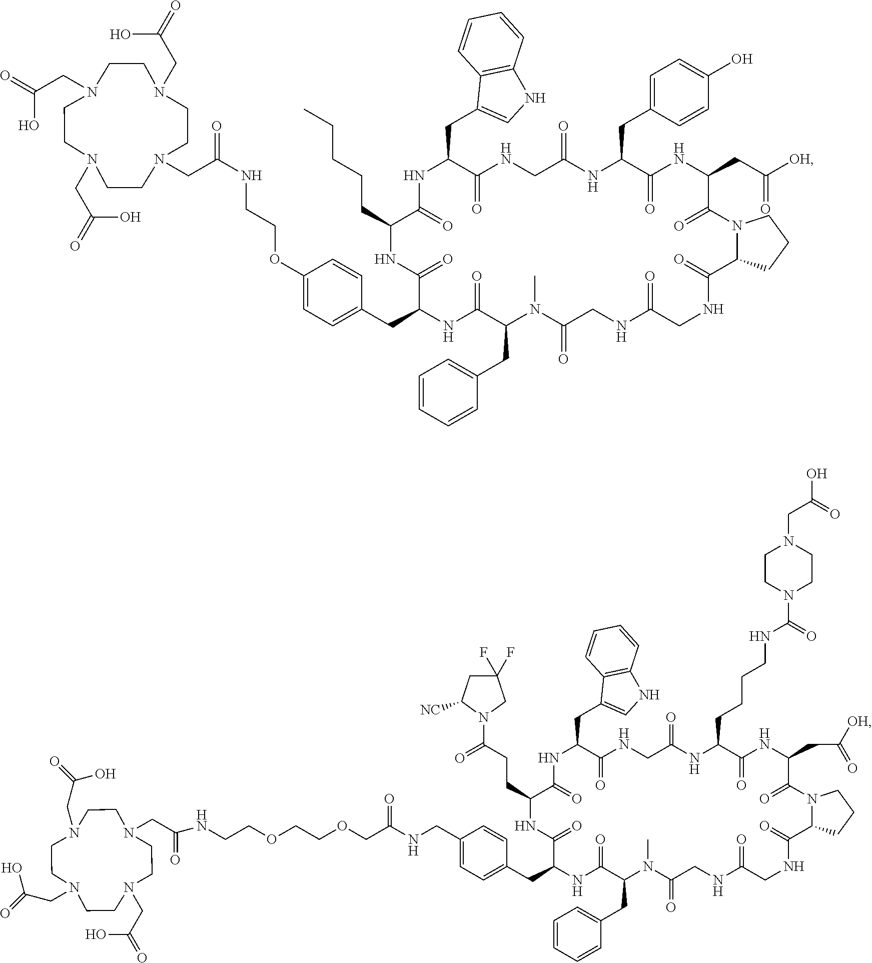 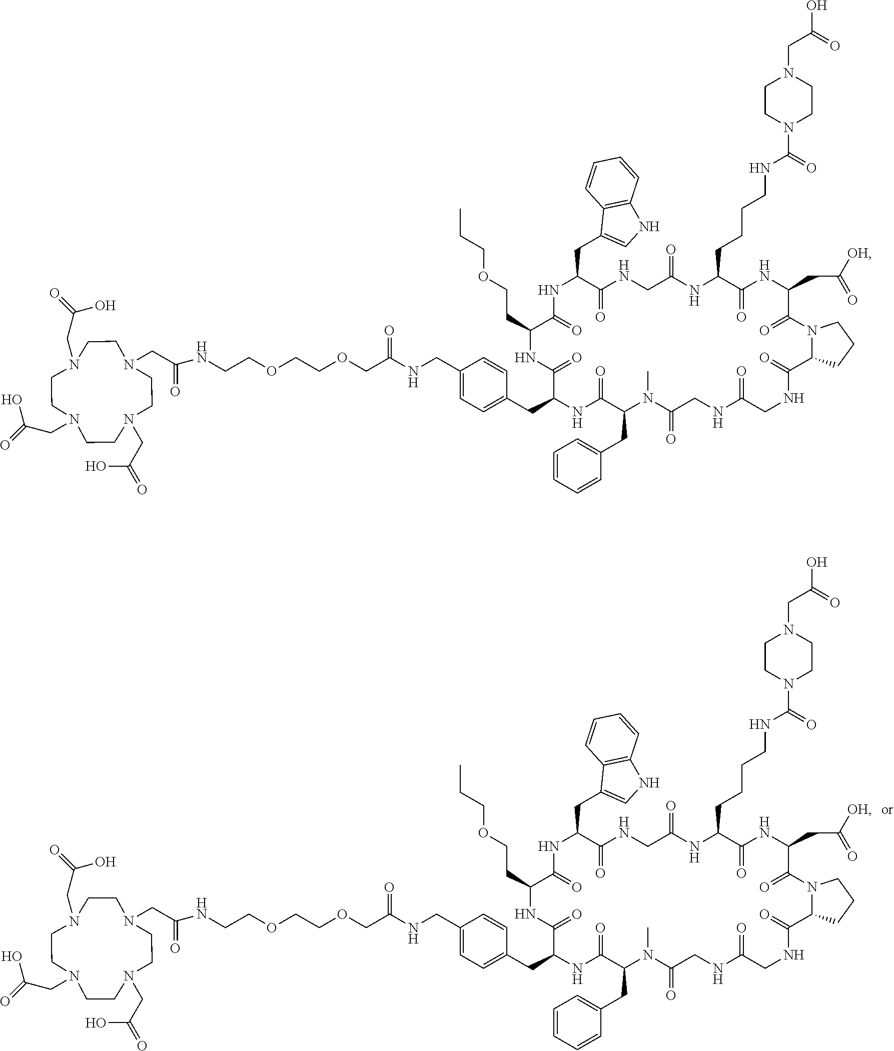 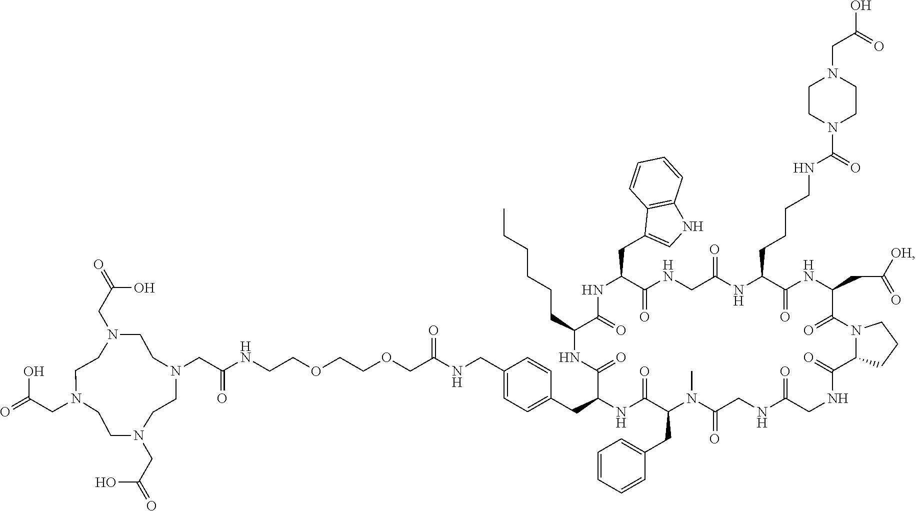 [SMILES: CCCCCC[C@@H]1NC(=O)[C@H](Cc2ccc(CNC(=O)COCCOCCNC(=O)CN3CCN(CC(=O)O)CCN(CC(=O)O)CCN(CC(=O)O)CC3)cc2)NC(=O)[C@H](Cc2ccccc2)N(C)C(=O)CNC(=O)CNC(=O)[C@H]2CCCN2C(=O)[C@H](CC(=O)O)NC(=O)[C@H](CCCCNC(=O)N2CCN(CC(=O)O)CC2)NC(=O)CNC(=O)[C@H](Cc2c[nH]c3ccccc23)NC1=O]
or a pharmaceutically acceptable salt, solvate, or tautomer thereof, which is optionally radiolabeled with a radionuclide.

65-67. (canceled)

68. A pharmaceutical composition comprising a compound according to claim 1, or a pharmaceutically acceptable salt, solvate, stereoisomer, or tautomer thereof, and one or more pharmaceutically acceptable carriers or stabilizers.

69-72. (canceled)

73. A method of imaging cancer in a subject, comprising administering to the subject a compound according to claim 1, or a pharmaceutically acceptable salt, solvate, stereoisomer, or tautomer thereof or a pharmaceutical composition thereof.

74-75. (canceled)

76. A method of treating cancer in a subject, comprising administering to the subject a therapeutically effective amount of a compound according to claim 1 or a pharmaceutical composition thereof.

77. (canceled)

78. The method of claim 76, wherein the cancer is selected from bladder cancer, breast cancer, cholangiocarcinoma, colon cancer, colorectal cancer, endocrine cancer, epithelial cancer, glioblastoma, head and/or neck cancer, mesothelioma, nasopharyngeal cancer, lung cancer, ovarian cancer, pancreatic cancer, prostate cancer, testicular cancer, thyroid cancer, and sarcoma.

79. (canceled)

80. A cyclized peptide (P): 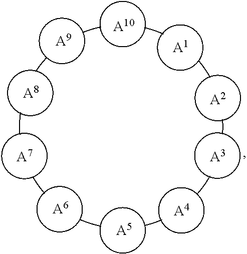
or a pharmaceutically acceptable salt, solvate, stereoisomer, or tautomer thereof, wherein:
A¹ is 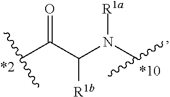
wherein
R^{1a} is selected from the group consisting of H and C₁₋₃-alkyl;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1b} is optionally substituted with 1, 2, 3, or 4 substituents independently selected from the group consisting of C₁₋₃-alkyl, —NH₂, —NHC₁₋₆-alkyl, —N(C₁₋₆-alkyl)₂, —OH, halo, and C(O)R^{1d};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH;
R^{1d} is selected from the group consisting of H, —OH, C₁₋₆-alkyl —NHC₁₋₆-alkyl, and —OC₁₋₆-alkyl; and
*2 indicates the point of attachment to A² and *10 indicates the point of attachment to A¹⁰;
A² is an amino acid or derivative thereof,
A³ is 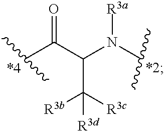
wherein
R^{3a} is selected from the group consisting of H and C₁₋₃-alkyl;
R^{3b} is selected from the group consisting of H, C₁₋₃-alkyl, and halo;
R^{3c} is selected from the group consisting of halo, C₁₋₆ alkyl, C₃₋₈-cycloalkyl, phenyl, and 5-6 membered heteroaryl, wherein the C₁₋₆ alkyl, phenyl, and 5-6 membered heteroaryl of R³⁰ are optionally substituted with 1, 2, 3, or 4 substituents independently selected from the group consisting of halo, —C(O)OH, —C(O)C₁₋₆-alkyl, —OH, —NHC₁₋₆-alkyl, —N(C₁₋₆-alkyl)₂, and —NH₂;
R^{3d} is selected from the group consisting of H, C₁₋₃-alkyl, and halo; and
*2 indicates the point of attachment to A² and *4 indicates the point of attachment to A⁴;
A⁴ is 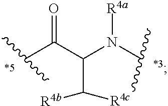
wherein
R^{4a} is selected from the group consisting of H and C₁₋₃-alkyl;
R^{4b} is selected from the group consisting of H and C₁₋₃-alkyl;
R^{4c} is selected from the group consisting of phenyl and 5-6 membered heteroaryl wherein phenyl and 5-6 membered heteroaryl of R^{4c} are optionally substituted with 1, 2, 3, 4, or 5 substituents R^{4d};
each R^{4d} is independently selected from C₁₋₆-alkyl, —OH, —OC₁-C₆-alkyl, —NH₂, halo, —NHC(O)R^{4f}, —C(O)NHR^{4f}, and —C(O)OR^{4f}, wherein the C₁₋₆-alkyl and —OC₁-C₆-alkyl of R^{4d} are each optionally substituted with 1, 2, or 3 substituents R^{4e};
each R^{4e} is independently selected from —CN, —NH₂, —NHC₁₋₆-alkyl, —N(C₁₋₆-alkyl)₂, —NHC(O)—(C₁₋₆-alkyl), —N₃, —OH, —C(O)OH, —C(O)NH₂, and 5-6 membered heteroaryl, wherein the 5-6 membered heteroaryl of R⁴ is optionally substituted with —C₁₋₆-alkyl-NH₂ or —C₁₋₃-alkyl-OH;
each R^{4f} is independently selected from the group consisting of H and C₁₋₆-alkyl; and
*3 indicates the point of attachment to A³ and *5 indicates the point of attachment to A⁵;
A⁵ is 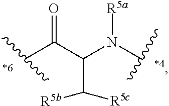
wherein
R^{5a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of
R^{5c} is optionally substituted with halo, —OH, —OC₁₋₆-alkyl, and —C(O)R^{5d};
R^{5d} is 4-7 membered heterocycloalkyl optionally substituted with 1, 2, 3, or 4 groups independently selected from C₁₋₃-alkyl, —CN, halo, and —OH; and
wherein *4 indicates the point of attachment to A⁴ and *6 indicates the point of attachment to A⁶;
A⁶ is 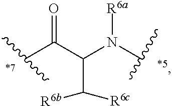
wherein
R^{6a} is selected from the group consisting of H and C₁₋₃-alkyl;
R^{6b} is selected from the group consisting of H and C₁₋₃-alkyl;
R^{6c} is a 5 to 10 membered heteroaryl optionally substituted with 1, 2, 3, or 4 groups independently selected from C₁₋₃-alkyl, —CN, halo, —OH, and —OC₁₋₆-alkyl; and
wherein *5 indicates the point of attachment to A⁵ and *7 indicates the point of attachment to A⁷;
A⁷ is an amino acid or derivative thereof,
A⁸ is 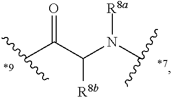
wherein
R^{8a} is selected from the group consisting of H, C₁₋₃-alkyl, and —CH₂-phenyl, wherein the C₁₋₃-alkyl and —CH₂-phenyl of R^{8a} are each optionally substituted with 1, 2, or 3 substituents independently selected from —NH₂, —NHC₁₋₆-alkyl, —N(C₁₋₆-alkyl)₂, —OH, —OC₁₋₃-alkyl, and C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R^{8c}, —C₁₋₆-alkyl-O—C(O)R^{8c}, and —C₁₋₆-alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH; and
wherein *7 indicates the point of attachment to A⁷ and *9 indicates the point of attachment to A⁹;
A⁹ is an amino acid or derivative thereof; and
A¹⁰ is 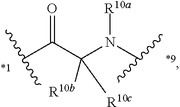
wherein
R^{10a} is selected from the group consisting of H and C₁₋₃-alkyl;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1, 2, 3, or 4 substituents independently selected from C₁₋₃-alkyl, —NH₂—NHC₁₋₆-alkyl, —N(C₁₋₆-alkyl)₂, —OH, halo, and C(O)R^{10d};
R^{10d} is selected from the group consisting of H, —OH, C₁₋₆-alkyl and —OC₁₋₆-alkyl; and
wherein *1 indicates the point of attachment to A¹ and *9 indicates the point of attachment to A⁹.

81. The cyclized peptide (P) of claim 80, or a pharmaceutically acceptable salt, solvate, stereoisomer, or tautomer thereof, wherein:
A¹ is 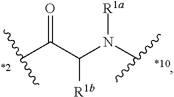
wherein
R^{1a} is selected from the group consisting of H and C₁₋₃-alkyl;
R^{1b} is selected from the group consisting of H and C₁₋₆-alkyl, wherein the C₁₋₆-alkyl of R^{1b} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{1c};
or R^{1a} and R^{1b} are optionally connected to form a 4-7 membered heterocycloalkyl, wherein the 4-7 membered heterocycloalkyl formed by R^{1a} and R^{1b} is optionally substituted with 1, 2, 3, or 4 substituents independently selected from the group consisting of C₁₋₃-alkyl, —NH₂, —NHC₁₋₆-alkyl, —N(C₁₋₆-alkyl)₂, —OH, halo, and C(O)R^{1d};
R^{1c} is selected from the group consisting of C₁₋₆-alkyl and 4-7 membered heterocycloalkyl, wherein the 4-7 membered heterocycloalkyl of R^{1c} is optionally substituted with 1, 2, 3, or 4 substituents independently selected from the group consisting of —C₁₋₆-alkyl-NH₂ and —C₁₋₆-alkyl-C(O)OH;
R^{1d} is selected from the group consisting of H, —OH, C₁₋₆-alkyl, —NHC₁₋₆-alkyl, and —OC₁₋₆-alkyl; and
*2 indicates the point of attachment to A² and *10 indicates the point of attachment to A¹⁰;
A² is 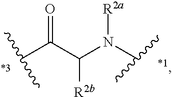
wherein
R^{2a} is selected from the group consisting of H and C₁₋₃-alkyl;
R^{2b} is selected from the group consisting of H and C₁₋₆-alkyl, wherein the C₁₋₆-alkyl of R^{2b} is optionally substituted with 1, 2, 3, or 4 substituents independently selected from the group consisting of —C(O)OH, —OH, —NH₂, —NHC₁₋₆-alkyl, and —N(C₁₋₆-alkyl)₂; and
*1 indicates the point of attachment to A¹ and *3 indicates the point of attachment to A³;
A³ is 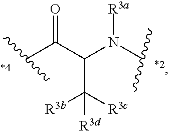
wherein
R^{3a} is selected from the group consisting of H and C₁₋₃-alkyl;
R^{3b} is selected from the group consisting of H, C₁₋₃-alkyl, and halo;
R^{3c} is selected from the group consisting of halo, C₁₋₆ alkyl, C₃₋₈-cycloalkyl, phenyl, and 5-6 membered heteroaryl, wherein the C₁₋₆ alkyl, phenyl, and 5-6 membered heteroaryl of R^{3c} are optionally substituted with 1, 2, 3, or 4 substituents independently selected from the group consisting of halo, —C(O)OH, —C(O)C₁₋₆-alkyl, —OH, —NHC₁₋₆-alkyl, —N(C₁₋₆-alkyl)₂, and —NH₂;
R^{3d} is selected from the group consisting of H, C₁₋₃-alkyl, and halo; and
*2 indicates the point of attachment to A² and *4 indicates the point of attachment to A⁴;
A⁴ is 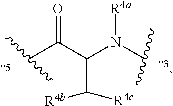
wherein
R^{4a} is selected from the group consisting of H and C₁₋₃-alkyl;
R^{4b} is selected from the group consisting of H and C₁₋₃-alkyl;
R^{4c} is selected from the group consisting of phenyl and 5-6 membered heteroaryl wherein phenyl and 5-6 membered heteroaryl of R^{4c} are optionally substituted with 1, 2, 3, 4, or 5 substituents R^{4d};
each R^{4d} is independently selected from C₁₋₆-alkyl, —OH, —OC₁-C₆-alkyl, —NH₂, halo, —NHC(O)R^{4f}, —C(O)NHR^{4f}, and —C(O)OR^{4f}, wherein the C₁₋₆-alkyl and —OC₁-C₆-alkyl of R^{4d} are each optionally substituted with 1, 2, or 3 substituents R^{4c};
each R^{4c} is independently selected from —CN, —NH₂, —NHC₁₋₆-alkyl, —N(C₁₋₆-alkyl)₂, —NHC(O)—(C₁₋₆-alkyl), —N₃, —OH, —C(O)OH, —C(O)NH₂, and 5-6 membered heteroaryl, wherein the 5-6 membered heteroaryl of R^{4c} is optionally substituted with —C₁₋₆-alkyl-NH₂ or —C₁₋₃-alkyl-OH;
each R^{4′} is independently selected from the group consisting of H and C₁₋₆-alkyl; and
*3 indicates the point of attachment to A³ and *5 indicates the point of attachment to A⁵;
A⁵ is 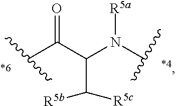
wherein
R^{5a} is selected from the group consisting of H and C₁₋₃-alkyl;
R^{5b} is selected from the group consisting of H, C₁₋₃-alkyl, and —OH;
R^{5c} is selected from the group consisting of H and C₁₋₈-alkyl, wherein the C₁₋₈-alkyl of R^{5c} is optionally substituted with halo, —OH, —OC₁₋₆-alkyl, and —C(O)R^{5d};
R^{5d} is 4-7 membered heterocycloalkyl optionally substituted with 1, 2, 3, or 4 groups independently selected from C₁₋₃-alkyl, —CN, halo, and —OH; and
wherein *4 indicates the point of attachment to A⁴ and *6 indicates the point of attachment to A⁶;
A⁶ is 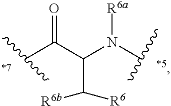
wherein
R^{6a} is selected from the group consisting of H and C₁₋₃-alkyl;
R^{6b} is selected from the group consisting of H and C₁₋₃-alkyl;
R^{6c} is a 5-10 membered heteroaryl optionally substituted with 1, 2, 3, or 4 groups independently selected from C₁₋₃-alkyl, —CN, halo, —NH₂, —NHC₁₋₆-alkyl, —N(C₁₋₆-alkyl)₂, —OH, and —OC₁₋₆-alkyl; and
wherein *5 indicates the point of attachment to A⁵ and *7 indicates the point of attachment to A⁷;
A⁷ is 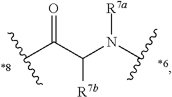
wherein
R^{7a} is selected from the group consisting of H and C₁₋₃-alkyl;
R^{7b} is selected from the group consisting of H and C₁₋₆-alkyl; and
wherein *6 indicates the point of attachment to A⁶ and *8 indicates the point of attachment to A⁸;
A⁸ is 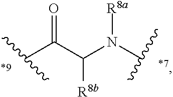
wherein
R^{8a} is selected from the group consisting of H, C₁₋₃-alkyl, and —CH₂-phenyl, wherein the C₁₋₃-alkyl and —CH₂-phenyl of R^{8a} are each optionally substituted with 1, 2, or 3 substituents independently selected from —NH₂, —NHC₁₋₆-alkyl, —N(C₁₋₆-alkyl)₂, —OH, —OC₁₋₃-alkyl, and C₁₋₃-alkyl;
R^{8b} is selected from the group consisting of H, C₁₋₆-alkyl, —C₁₋₆-alkyl-phenyl, —C₁₋₆-alkyl(5-6 membered heteroaryl), —C₁₋₆-alkyl-CH₂—C(O)R⁸′, —C₁₋₆-alkyl-O—C(O)R⁸′, and —C₁₋₆-alkyl-NH—C(O)R^{8c}, wherein the C₁₋₆-alkyl, —C₁₋₆-alkyl-phenyl, and —C₁₋₆-alkyl(5-6 membered heteroaryl) of R^{8b} are optionally substituted with 1, 2, or 3 substituents independently selected from halo, —NH₂, —NHC₁₋₆-alkyl, —N(C₁₋₆-alkyl)₂, —OH, —OC₁₋₃-alkyl, and C₁₋₃-alkyl;
R^{8c} is a 4-7 membered heterocycloalkyl optionally substituted with —C₁₋₆-alkylC(O)OH or —C(O)OH; and
wherein *7 indicates the point of attachment to A⁷ and *9 indicates the point of attachment to A⁹;
A⁹ is 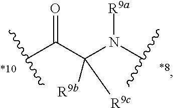
wherein
R^{9a} is selected from the group consisting of H and C₁₋₃-alkyl;
R^{9b} is selected from the group consisting of H and C₁₋₃-alkyl;
R^{9c} is C₁₋₆-alkyl optionally substituted with 1, 2, 3, or 4 groups independently selected from —CN, —C(O)OH, —C(O)NH₂, halo, —NH₂, —NHC₁₋₆-alkyl, —N(C₁₋₆-alkyl)₂, —NHC(O)C₁₋₆-alkyl, —OH, and —OC₁₋₆-alkyl; and
wherein *8 indicates the point of attachment to A⁸ and *10 indicates the point of attachment to A¹⁰; and
A¹⁰ is 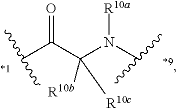
wherein
R^{10a} is selected from the group consisting of H and C₁₋₃-alkyl;
R^{10b} is selected from the group consisting of H and C₁₋₃-alkyl;
R^{10c} is selected from the group consisting of H and C₁₋₆-alkyl, wherein the C₁₋₆-alkyl of R^{10c} is optionally substituted with 1, 2, 3, or 4 substituents independently selected from the group consisting of —C(O)OH, —OH, —NH₂, —NHC₁₋₆-alkyl, —N(C₁₋₆-alkyl)₂, and —NH—C(O)R^{10d};
or R^{10a} and R^{10c} are optionally connected to form a 4-7 membered heterocycloalkyl, wherein the 4-7 membered heterocycloalkyl formed by R^{10a} and R^{10c} is optionally substituted with 1, 2, 3, or 4 substituents independently selected from C₁₋₃-alkyl, —NH₂, —NHC₁₋₆-alkyl, —N(C₁₋₆-alkyl)₂, —OH, halo, and C(O)R^{10d};
R^{10d} is selected from the group consisting of H, —OH, C₁₋₆-alkyl and —OC₁₋₆-alkyl; and
wherein *1 indicates the point of attachment to A¹ and *9 indicates the point of attachment to A⁹.

82-94. (canceled)
